(19) United States
(12) Patent Application Publication
(10) Pub. No.: US 2026/0327224 A1
(43) Pub. Date: Oct. 22, 2026

(54) SUBSTITUTED PYRIDOTRIAZOLE COMPOUND, AND PREPARATION METHOD THEREFOR AND USE THEREOF

(71) Applicant: ORIGIANT PHARMACEUTICAL CO., LTD., Chengdu, Sichuan (CN)

(72) Inventors: Shengyong YANG, Chengdu, Sichuan (CN); Linli LI, Chengdu, Sichuan (CN); Zhicheng JIANG, Chengdu, Sichuan (CN); Jichao WU, Chengdu, Sichuan (CN); Yuanhai ZHONG, Chengdu, Sichuan (CN); Baosong GUO, Chengdu, Sichuan (CN); Junhui DU, Chengdu, Sichuan (CN); Jiayu XIE, Chengdu, Sichuan (CN); Xiaocai YAN, Chengdu, Sichuan (CN); Shuang MA, Chengdu, Sichuan (CN); Xiaoquan WU, Chengdu, Sichuan (CN); Yan YU, Chengdu, Sichuan (CN); Qi HUANG, Chengdu, Sichuan (CN)

(21) Appl. No.: 19/520,436

(22) PCT Filed: Sep. 9, 2024
(86) PCT No.: PCT/CN2024/117673

§ 371 Date: Mar. 18, 2026

(30) Foreign Application Priority Data
Sep. 19, 2023 (CN) 202311218595.3
Jul. 12, 2024 (CN) 202410939370.5

(51) Int. Cl. C07D 471/04; A61K 31/437; A61K 31/438; A61K 31/439; A61K 31/444; A61K 31/4545; A61K 31/496; A61K 31/5377; A61K 31/5386; A61K 31/541; A61K 31/551; C07D 519/00

(52) U.S. Cl. CPC C07D 471/04; A61K 31/437; A61K 31/438; A61K 31/439; A61K 31/444; A61K 31/4545; A61K 31/496; A61K 31/5377; A61K 31/5386; A61K 31/541; A61K 31/551; C07D 519/00

(57) ABSTRACT

The present invention relates to the field of pharmaceutical technology, and specifically to substituted pyridotriazole compounds and the preparation method and use thereof. The present description discloses substituted pyridotriazole compounds having an inhibitory effect on RIPK1 and/or MLK, or a pharmaceutically acceptable salt, stereoisomer, solvate or prodrug thereof. The compound is represented by formula (I), wherein the definitions of respective groups in the formula are detailed in the description. In addition, the present invention further discloses a pharmaceutical composition comprising the compound, and use thereof in the preparation of a medicine for treating RIPK1-related diseases or disorders.

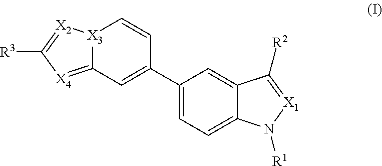

The present application claims priority to Chinese Patent Application No. 2023112185953 filed on Sep. 19, 2023, and Chinese Patent Application No. 2024109393705 filed on Jul. 12, 2024, which are incorporated herein by reference in their entireties.

### TECHNICAL FIELD

The present invention relates to the field of pharmaceutical technology, and specifically to substituted pyridotriazole derivatives and use thereof as RIPK1 and/or MLK inhibitors.

### BACKGROUND ART

Necroptosis (programmed necrosis) is a novel caspase-independent form of programmed cell death distinct from apoptosis, which has been discovered in recent years. It is regulated by death signals and exhibits necrosis-like structural characteristics. Compared with apoptosis, necroptosis does not form apoptotic bodies or exhibit chromatin condensation; compared with necrosis, necroptosis is a controlled mode of cell death regulated by multiple genes. In an in vitro culture system supplemented with the caspase inhibitor Z-VAD-FMK, TNF can induce necroptosis. The cells exhibit morphological characteristics of necrosis, including swelling, rupture, and the release of cellular contents, which subsequently trigger inflammatory and immune responses. In addition to TNF, ligands of TLR3 and TLR4, as well as certain bacterial and viral infections, can induce necroptosis.

Receptor-interacting protein kinase 1 (RIPK1) is a protein exhibiting specific serine/threonine kinase activity; it possesses an N-terminal kinase domain similar to that of other protein kinases, but features a distinct binding domain. Studies indicate that RIPK1 is a key mediator of necroptosis, modulating this process via the RIPK1/RIPK3/MLKL signaling axis. Various death receptors, such as TNFR, FAS, TRAILR, and Toll-like receptors, can trigger upstream signaling of necroptosis upon stimulation by inflammatory factors or exogenous infections. Under conditions where cIAPs and caspase activity are restricted, RIPK1 interacts with RIPK3 to form a necrosome. RIPK3 further recruits MLKL. Phosphorylated MLKL self-oligomerizes and migrates to the cell membrane to “punch holes,” causing the leakage of cellular contents and the disruption of ionization balance, ultimately leading to cell necrosis.

Necroptosis is closely associated with the pathogenesis and progression of inflammation, autoimmune diseases, neurodegenerative diseases, and tumors. For instance, studies indicate that necroptosis is a significant cause of Systemic Inflammatory Response Syndrome (SIRS). SIRS is a systemic inflammatory response syndrome triggered by infectious or non-infectious factors, resulting in a dysregulated and self-destructive reaction in the body. Critically ill patients are prone to SIRS due to diminished compensatory anti-inflammatory capabilities and metabolic dysfunction. Long-term studies have demonstrated that the TNF-α-induced SIRS model is highly correlated with RIPK1-dependent necroptosis. Inflammatory bowel diseases (IBD) are abnormal immune-mediated intestinal inflammation caused by environmental, genetic, infectious, and immunological factors; it is a chronic, non-specific inflammatory disease of the intestine. The pathogenesis remains unclear, but excessive apoptosis of intestinal epithelial cells, damage to the intestinal mucosal barrier, and increased permeability of intestinal epithelial cells are considered to be among the causes of IBD. Studies have shown that necroptosis plays an important role in the pathogenesis of IBD. Furthermore, necroptosis plays a significant role in the pathogenesis of various autoimmune diseases, such as rheumatoid arthritis (RA), psoriasis, and multiple sclerosis (MS). Activated microglia play a key role in the development of Alzheimer's disease (AD); RIPK1 is highly expressed in microglia, and RIPK1 inhibitors can effectively protect against Aβ-induced neuronal necroptosis in vitro. In addition to AD, necroptosis is also involved in the onset and progression of numerous neurodegenerative diseases, such as amyotrophic lateral sclerosis (ALS), frontotemporal dementia (FTD), and Parkinson's disease (PD). In 2016, Strilic et al. first revealed that tumor cells can induce necroptosis in vascular endothelial cells. Subsequently, tumor cells penetrate the vascular walls and achieve distant metastasis via blood circulation, which is a major cause of tumor metastasis. Experiments have shown that RIPK1 inhibitors can effectively inhibit tumor metastasis. In addition, studies indicate that RIPK1 kinase can promote the differentiation of tolerogenic macrophages within the pancreatic tumor microenvironment, whereas RIPK1 inhibition facilitates the differentiation of immunogenic macrophages in the pancreatic tumor microenvironment, thereby leading to adaptive immune activation and tumor protection. Mixed lineage kinase (MLK) is a MAPK kinase that targets JNK and p38 MAPK, activating them in response to various stimuli that induce cellular stress. Thus, MLK regulates a wide range of cellular processes. The MLK family consists of six members: MLK1, MLK2, MLK3, MLK6, dual leucine zipper kinase (DLK), and leucine zipper-bearing kinase (LZK). MLK2 is also known as MST. MLK3 is the most widely expressed member of the MLK family and is present in neurons and resident mononuclear phagocytes in the brain.

MLK3 can regulate neuroinflammation by influencing cytokines produced by microglia and brain macrophages. In particular, CEP-1347-mediated MLK3 inhibition results in a significant reduction in tumor necrosis factor alpha (TNF-α) and interleukin (IL)-6 released by Tat-induced macrophages and microglia. The potent brain-penetrant MLK3 inhibitor URMC-099 has been shown to be effective in vivo in protecting normal synaptic structure and reversing neuroinflammation following CNS exposure to HIV-1 Tat (Journal of Neuroscience the Official Journal of the Society for Neuroscience 33.24(2013):9998-10010.).

Blocking the MLK3 pathway may interfere with the neurotoxic effects of D-amyloid oligomers, and inhibiting the C-jun/JNK pathway blocks tau hyperphosphorylation in cultured hippocampal neurons (Journal of Neuroscience, 2009, 29(28):9078-9089). Therefore, MLK3 inhibitors may be valuable for the treatment of AD and other neurodegenerative diseases involving the C-jun/JNK pathway.

MLK3 inhibitors may also be used for the treatment of various cancers, such as pancreatic and breast cancers. MLK3 regulates the proliferation of certain tumor cell types that overexpress MLK3, including human schwannoma and meningioma cells, and breast cancer cells (Oncogene 29.31(2010):4399-4411).

In summary, RIPK1 and MLK are important therapeutic targets for necroptosis-related diseases such as inflammation, autoimmune diseases, neurodegenerative diseases and tumors, and RIPK1 inhibitors are expected to serve as potential therapeutic drugs for these diseases.

### SUMMARY OF INVENTION

An object of the present invention is to provide a class of structurally novel RIPK1 kinase inhibitors.

A first aspect of the present invention provides a compound of formula (I), or a pharmaceutically acceptable salt, solvate or stereoisomer thereof:

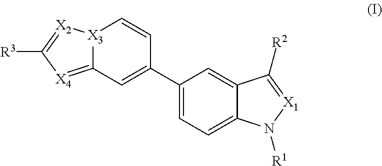

wherein,
X1 is selected from N or CH;
X2. X3 and X4 are each independently selected from N or CH, provided that at least one of X1, X2 and X3 is N;
R¹ is selected from C₁₋₆ alkyl, C₁₋₆ alkoxy, —C₁₋₆ alkyl-C₁₋₆ alkoxy or C₃₋₆ cycloalkyl, wherein the said C₁₋₆ alkyl is optionally substituted with halogen;
R² is selected from

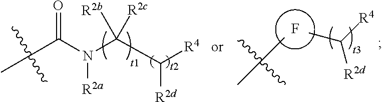

wherein, R⁴ is selected from C₁₋₆ alkyl, C₅₋₈ aryl, 8- to 12-membered partially unsaturated bicyclic group and 5- or 6-membered monocyclic heteroaryl, wherein the said C₅₋₈ aryl, 8- to 12-membered partially unsaturated bicyclic group and 5- or 6-membered monocyclic heteroaryl are optionally substituted with one or more substituents selected from R⁴¹;
R⁴¹ is selected from halogen, cyano, —NR^{4a}R^{4b}, C₁₋₆ alkyl and C₃₋₆ cycloalkyl;
Ring F is selected from 5- or 6-membered monocyclic heteroaryl and 4- to 8-membered nitrogen-containing partially unsaturated heterocyclyl;
R^{2a}. R^{2b}, R^{2c} and R^{2d} are each independently selected from hydrogen, deuterium, halogen, C₁₋₆ alkyl and C₃₋₆ cycloalkyl, wherein the said C₁₋₆ alkyl and C₃₋₆ cycloalkyl are optionally substituted with one or more halogen or hydroxyl;
Alternatively, R^{2b} and R^{2c}, together with the carbon atom to which they are attached, form C₃₋₆ cycloalkyl or 4- to 12-membered heterocycloalkyl;
Alternatively, R^{2a} and R^{2b}, together with the carbon atom and nitrogen atom to which they are attached, form 4- to 12-membered nitrogen-containing heterocycloalkyl or 4- to 8-membered nitrogen-containing partially unsaturated heterocyclyl;
Alternatively, R^{2b} and R^{2d}, together with the carbon atom to which they are attached, form C₃₋₆ cycloalkyl or 4- to 12-membered heterocycloalkyl;
The said C₃₋₆ cycloalkyl, 4- to 12-membered heterocycloalkyl, 4- to 12-membered nitrogen-containing heterocycloalkyl or 4- to 8-membered nitrogen-containing partially unsaturated heterocyclyl is optionally substituted with one or more substituents selected from halogen;
t1. t2 and t3 are each independently selected from integers of 0 to 6;
R³ is selected from 4- to 12-membered heterocycloalkyl, —NH—C₁₋₆ alkyl, —NH-4- to 12-membered heterocycloalkyl or —NH—C(O)R⁵, wherein the said C₁₋₆ alkyl or 4- to 12-membered heterocycloalkyl is optionally substituted with one or more C₃₋₆ cycloalkyl;
R⁵ is selected from hydrogen, deuterium, C₁₋₆ alkyl, C₁₋₆ alkoxy, C₃₋₆ cycloalkyl, 4- to 12-membered heterocycloalkyl, 5- or 6-membered heteroaryl, —NR^{5a}R^{5b}, —C(O)O—C₁₋₆ alkyl, —C(O)NR^{5a}R^{5b}, —NR^{5a}R^{5b}—C(O)O—C₁₋₆ alkyl, —C(O)-4- to 12-membered heterocycloalkyl, —C₁₋₆ alkyl-(OCH₂CH₂)ₘOCH₃, —C₁₋₆ alkyl-NR^{5a}R^{5b}, —C₁₋₆ alkyl-NH—C₁₋₆ alkyl-O—C₁₋₆ alkyl, —C₁₋₆ alkyl-4- to 12-membered heterocycloalkyl, —C₁₋₆ alkyl-NH-4- to 12-membered heterocycloalkyl, —C₁₋₆ alkyl-NH—C₁₋₆ alkyl-4- to 12-membered heterocycloalkyl, or —C₁₋₆ alkyl-5- or 6-membered heteroaryl, wherein the said C₁₋₆ alkyl, C₁₋₆ alkoxy, C₃₋₆ cycloalkyl, 4- to 12-membered heterocycloalkyl, and 5- or 6-membered heteroaryl are optionally substituted with one or more substituents selected from deuterium, halogen, cyano, oxo, hydroxyl, carboxyl, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl, or 4- to 12-membered heterocycloalkyl, wherein two hydrogen atoms on an optional carbon atom of the said C₁₋₆ alkyl are substituted to form a C₃₋₆ cycloalkyl; m is an integer selected from 1 to 4;
R^{4a}, R^{4b}, R^{5a} and R^{5b} are each independently selected from hydrogen, halogen, C₁₋₆ alkyl or C₁₋₆ alkoxy, wherein the said C₁₋₆ alkyl is optionally substituted with one or more substituents selected from halogen, hydroxyl or C₃₋₆ cycloalkyl.

In one embodiment, R¹ is selected from C₁₋₆ alkyl, C₁₋₆ alkoxy, —C₁₋₆ alkyl-C₁₋₆ alkoxy or C₃₋₆ cycloalkyl; preferably, R¹ is selected from C₁₋₆ alkyl or —C₁₋₃ alkyl-C₁₋₃ alkoxy;
preferably, R¹ is selected from methyl, ethyl or —CH₂CH₂OCH₃.

In one embodiment, t1, t2 and t3 are each independently selected from 0, 1, 2 or 3;

In one embodiment, R² is selected from

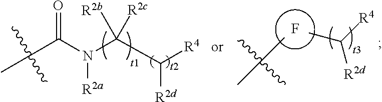

R⁴ is selected from C₁₋₆ alkyl,

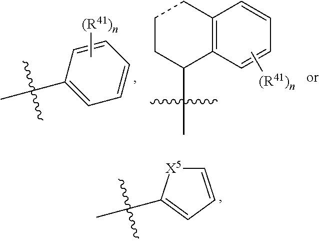

wherein the said R⁴¹ is as defined for the compound of formula (I); X⁵ is selected from N, O or S;
preferably, R⁴¹ is selected from halogen, cyano or amino;
preferably, R⁴¹ is selected from fluoro, chloro or cyano;
preferably, ring F is selected from a 5- or 6-membered monocyclic heteroaryl or a 4- to 8-membered nitrogen-containing partially unsaturated heterocyclyl, wherein the said 5- or 6-membered monocyclic heteroaryl or 4- to 8-membered nitrogen-containing partially unsaturated heterocyclyl optionally contains 1, 2 or 3 heteroatoms selected from N and O as ring atoms; preferably, ring F is selected from a 5- or 6-membered monocyclic heteroaryl or a 4- to 6-membered nitrogen-containing partially unsaturated heterocyclyl, wherein the said 5- or 6-membered monocyclic heteroaryl or 4- to 8-membered nitrogen-containing partially unsaturated heterocyclyl optionally contains 1, 2 or 3 heteroatoms selected from N and O as ring atoms; preferably, ring F is selected from triazolyl or dihydrooxazolyl.

In one embodiment, R^{2a}, R^{2b}, R^{2c} and R^{2d} are each independently selected from hydrogen, deuterium, halogen, C₁₋₆ alkyl, halogen-substituted C₁₋₆ alkyl or hydroxy-substituted C₁₋₆ alkyl; more preferably selected from hydrogen, deuterium, halogen, methyl, ethyl, fluoroethyl, difluoroethyl or hydroxymethyl;
preferably, R^{2a} is selected from hydrogen, deuterium or methyl;
preferably, R^{2b}, R^{2c} and R^{2d} are each independently selected from methyl, ethyl, fluoroethyl, difluoroethyl or hydroxymethyl; preferably, R^{2b} and R^{2c}, together with the carbon atom to which they are attached, form C₃₋₆ cycloalkyl or 4- to 12-membered heterocycloalkyl;
preferably, R^{2b} and R^{2c}, together with the carbon atom to which they are attached, form C₃₋₆ cycloalkyl or 4- to 12-membered heterocycloalkyl;
preferably, R^{2b} and R^{2c}, together with the carbon atom to which they are attached, form a 4- to 8-membered heterocycloalkyl, wherein the said 4- to 8-membered heterocycloalkyl optionally contains 1 or 2 heteroatoms selected from N, O and S as ring atoms;
preferably, the said 4- to 12-membered heterocycloalkyl is a 4- to 6-membered oxygen-containing heterocycloalkyl, more preferably oxetanyl;
Alternatively, R^{2a} and R^{2b}, together with the carbon atom and nitrogen atom to which they are attached, form a 4- to 12-membered nitrogen-containing heterocycloalkyl or a 4- to 8-membered nitrogen-containing partially unsaturated heterocyclyl, wherein the said 4- to 12-membered nitrogen-containing heterocycloalkyl is preferably azetidinyl or morpholinyl; the said 4- to 8-membered nitrogen-containing partially unsaturated heterocyclyl is preferably dihydropyrazolyl, and the said azetidinyl is optionally substituted with one or more halogens;
Alternatively, R^{2b} and R^{2d}, together with the carbon atom to which they are attached, form C₃₋₆ cycloalkyl or preferably cyclopropyl;
preferably, R² is selected from

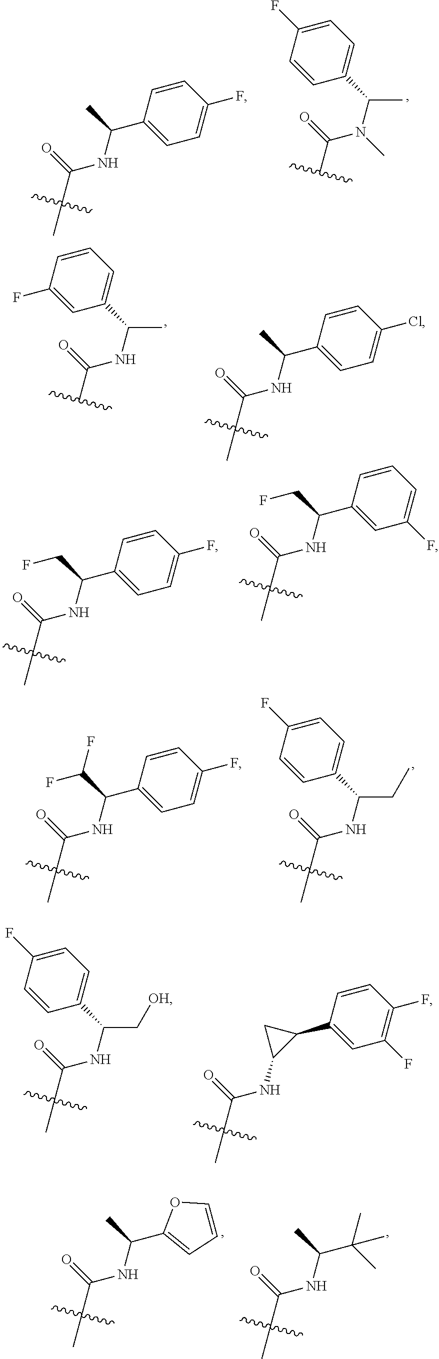

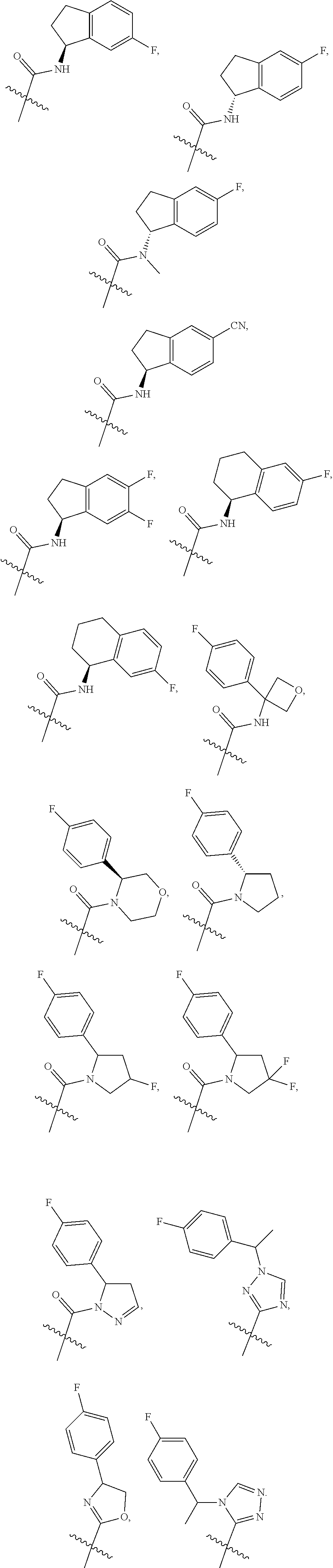

In one embodiment, R³ is selected from 4- to 6-membered heterocycloalkyl, —NH—C₁₋₆ alkyl, —NH-4- to 12-membered heterocycloalkyl or —NH—C(O)R⁵, wherein the said C₁₋₆ alkyl is optionally substituted with one or more C₃₋₆ cycloalkyl, and the said 4- to 6-membered heterocycloalkyl is optionally substituted with one or more oxo; preferably, R³ is selected from morpholinyl or oxomorpholinyl;
preferably, R³ is selected from —NH-isopropyl, —NH—CH₂-cyclopropyl or —NH-tetrahydrofuran-3-yl;
preferably, R⁵ is selected from methyl, ethoxy, —CH₂CH₂COOH, —CH₂Cl, —CH₂CN, —CH₂OCH₃, —CH(CH₃)OCH₃,

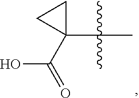 [SMILES: *C1(C(=O)O)CC1]

—C(O)OCH₂CH₃, —C(O)NH₂, —CH₂(OCH₂CH₂)₂OCH₃, —CH₂(OCH₂CH₂)₃OCH₃,

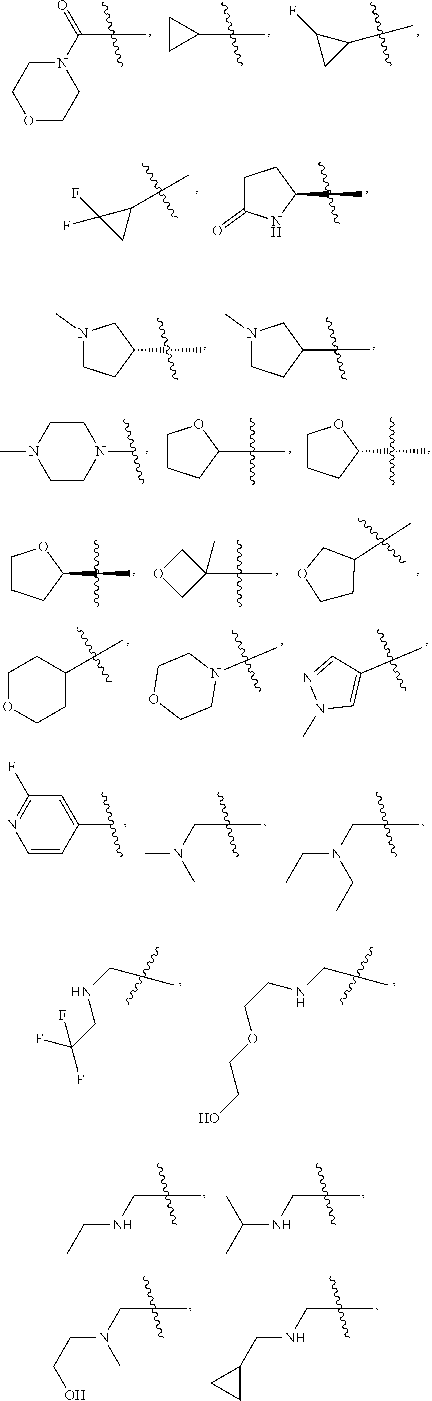

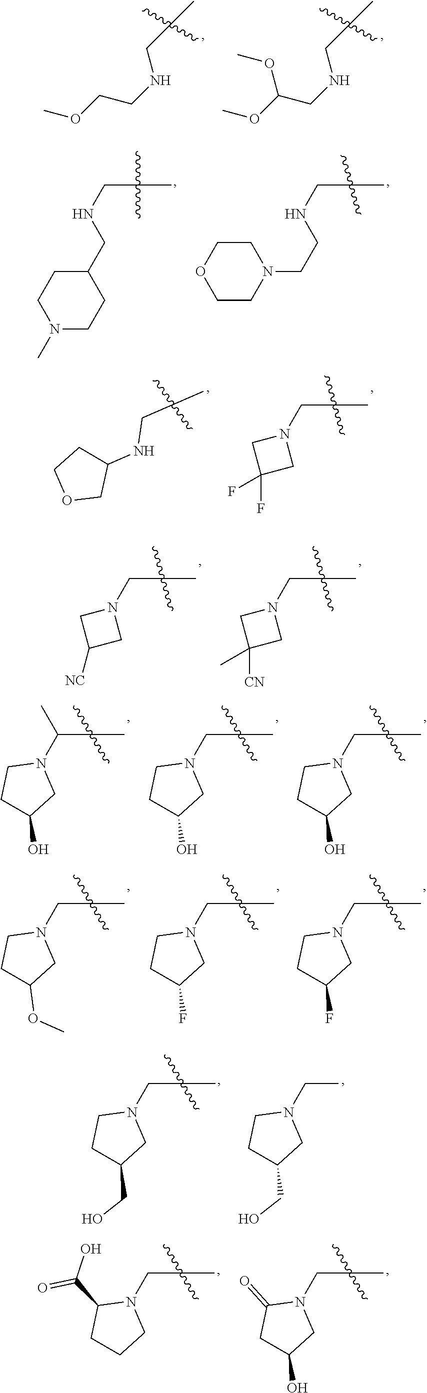

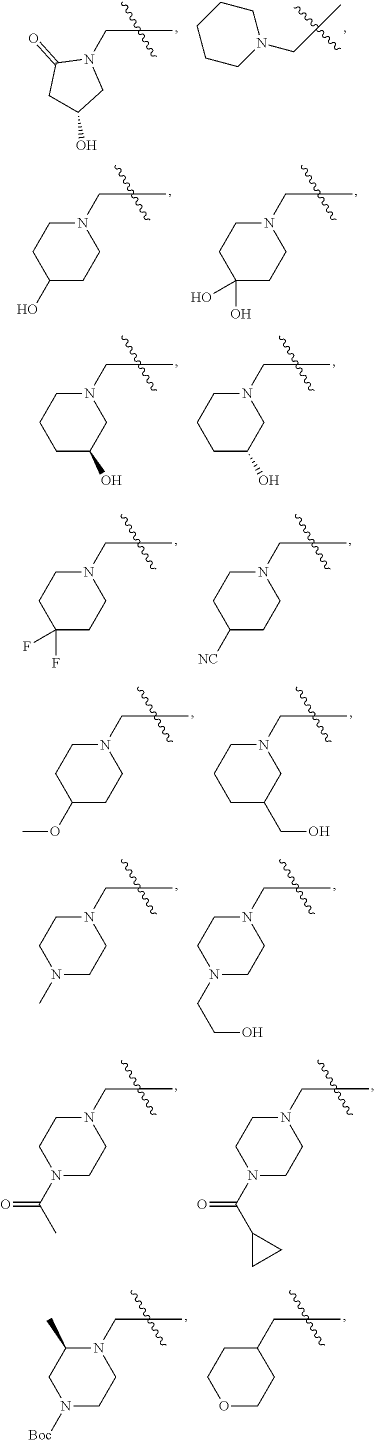

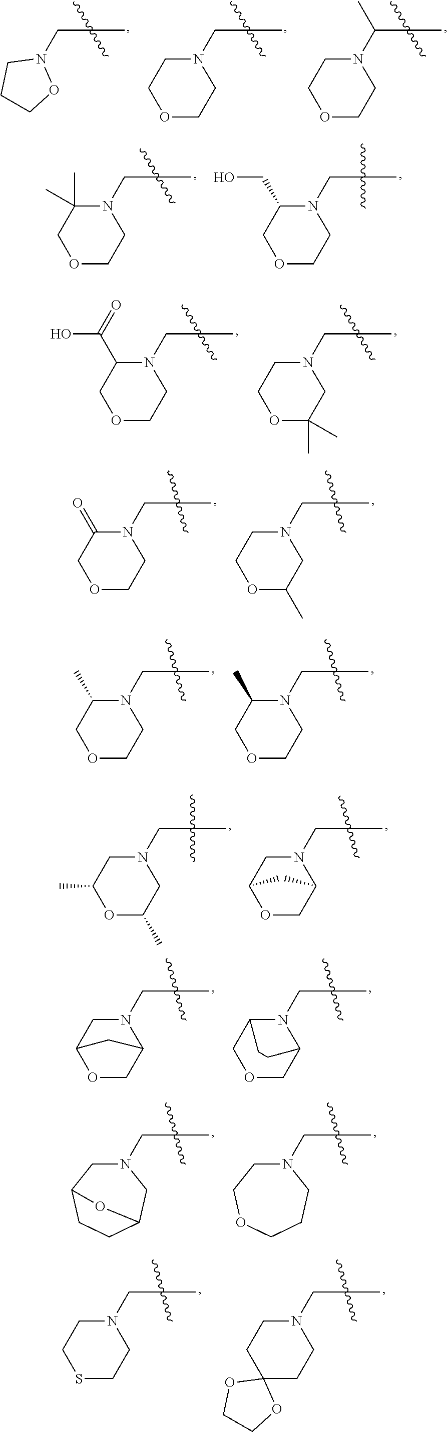

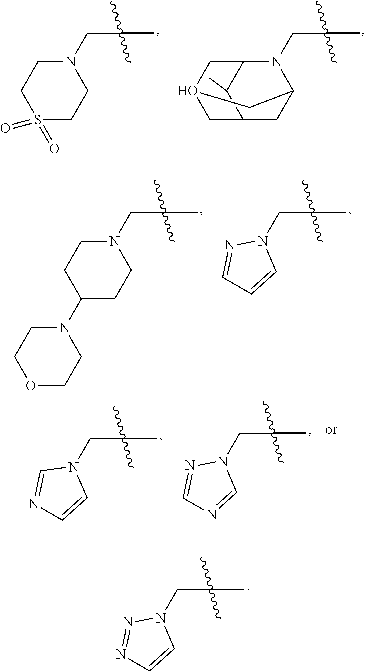

In one embodiment, R³ is selected from:

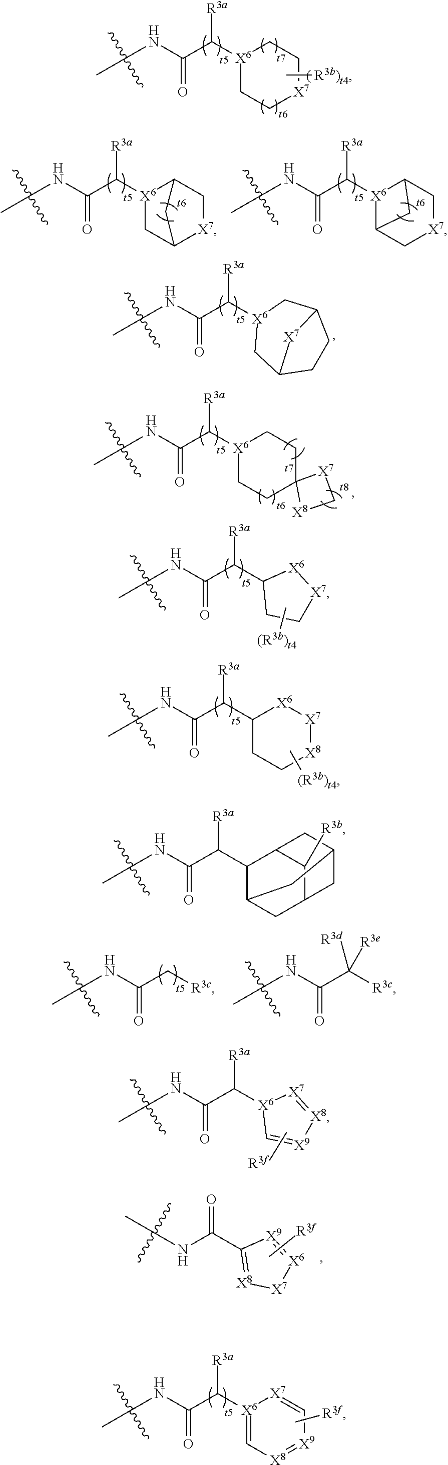

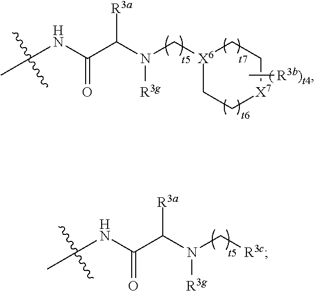

preferably R³ is selected from:

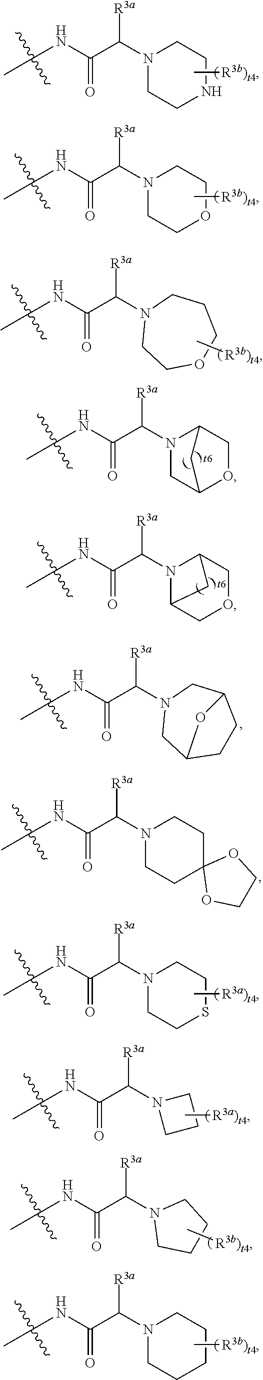

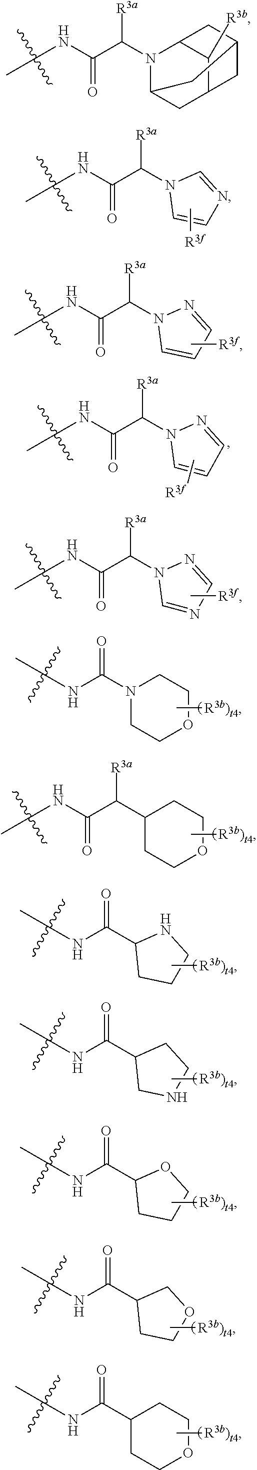

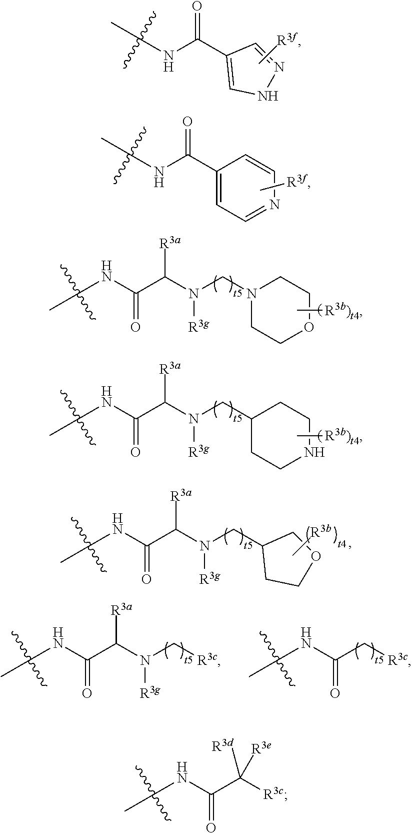

wherein, X⁶, X⁷, X⁸, and X⁹ are each independently selected from N, NH, O, S, CH or CH₂;
t4 and t5 are each independently selected from integers of 0 to 6;
t6 and t7 are each independently selected from 0, 1 or 2;
R^{3a}, R^{3b}, R^{3c}, R^{3d}, R^{3e}, R^{3f}, and R^{3g} are each independently selected from hydrogen, deuterium, halogen, cyano, oxo, hydroxyl, carboxyl, C₁₋₆ alkyl, C₁₋₆ alkoxy, C₃₋₆ cycloalkyl, 4- to 12-membered heterocycloalkyl, —(OCH₂CH₂)ₘOCH₃, —NR^{X3a}R^{X3b}, —C(O)R^{X3c}, wherein the said C₁₋₆ alkyl, C₁₋₆ alkoxy, C₃₋₆ cycloalkyl are optionally substituted with one or more substituents selected from halogen, hydroxyl, C₁₋₆ alkoxy;
or. R^{3d}, R^{3e} and the carbon atom to which they are attached together form C₃₋₆ cycloalkyl;
R^{X3a}, R^{X3b}, and R^{X3c} are each independently selected from hydrogen, C₁₋₆ alkyl, C₁₋₆ alkoxy or C₃₋₆ cycloalkyl;
m is an integer selected from 1 to 4; preferably 2 or 3.

In one embodiment, R^{3a} is selected from hydrogen, deuterium, oxo, or C₁₋₆ alkyl; preferably from hydrogen, oxo, or methyl; preferably, R^{3b} is selected from hydrogen, deuterium, halogen, cyano, oxo, hydroxyl, carboxyl, C₁₋₆ alkyl, C₁₋₆ alkoxy, —C₁₋₆ alkyl-OH, —C(O)C₁₋₆ alkyl, —C(O)C₃₋₆ cycloalkyl, C₃₋₆ cycloalkyl, or 4- to 12-membered heterocycloalkyl; wherein the said 4- to 12-membered heterocycloalkyl optionally contains 1, 2, or 3 heteroatoms selected from N and O as ring atoms;
preferably, R^{3b} is selected from hydrogen, deuterium, halogen, cyano, oxo, hydroxyl, carboxyl, methyl, methoxyl, —CH₂OH, —C(O)CH₃, —C(O)cyclopropyl, cyclopropyl, or morpholinyl;
preferably, R^{3c} is selected from hydrogen, deuterium, halogen, cyano, oxo, hydroxyl, carboxyl, amino, —(OCH₂CH₂)ₘOCH₃, —NR^{X3a}R^{X3b}, C₁₋₆ alkyl, C₁₋₆ alkoxy, halogen-substituted C₁₋₆ alkyl, C₃₋₆ cycloalkyl and halogen-substituted C₃₋₆ cycloalkyl, wherein the said C₁₋₆ alkyl or C₁₋₆ alkoxy is optionally substituted with one or more substituents selected from hydroxyl and C₁₋₆ alkoxy;
preferably, R^{3c} is selected from hydrogen, deuterium, halogen, cyano, oxo, hydroxyl, carboxyl, amino, dimethylamino, diethylamino, trifluoromethyl, methoxy, ethoxy, isopropyl, cyclopropyl, halogen-substituted cyclopropyl, —CH(OCH₃)₂, —OCH₂CH₂OH, —(OCH₂CH₂)₂OCH₃ or —(OCH₂CH₂)₃OCH₃;
Alternatively, R^{3d} and R^{3e} together with the carbon atom to which they are attached form C₃₋₆ cycloalkyl; preferably, R^{3d} and R^{3e} together with the carbon atom to which they are attached form cyclopropyl.

In one embodiment, R^{3f} is hydrogen.

In one embodiment, the compound of formula (I) is a compound represented by formula (II):

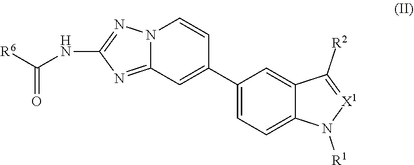

wherein X¹, R¹ and R² are each as defined for the compound of formula (I);
R⁶ is selected from hydrogen, deuterium, C₁₋₆ alkyl, C₁₋₆ alkoxy, C₃₋₆ cycloalkyl, 4- to 12-membered heterocycloalkyl, 5- or 6-membered heteroaryl, —C(O)NR^{5a}R^{5b}, —C(O)O—C₁₋₆ alkyl or —C(O)-(4- to 12-membered heterocycloalkyl), wherein the said C₁₋₆ alkyl, C₃₋₆ cycloalkyl, 4- to 12-membered heterocycloalkyl and 5- or 6-membered heteroaryl are optionally substituted with one or more substituents selected from halogen, oxo, hydroxyl, carboxyl, cyano, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl or 4- to 12-membered heterocycloalkyl, wherein two hydrogen atoms on any one carbon atom of the said C₁₋₆ alkyl are substituted to form C₃₋₆ cycloalkyl;
R^{5a} and R^{5b} are each independently selected from hydrogen, C₁₋₆ alkyl or C₁₋₆ alkoxy, wherein the said C₁₋₆ alkyl is optionally substituted with one or more halogen or C₃₋₆ cycloalkyl.

In one embodiment, R⁶ is selected from methyl, ethoxy, —CH₂CH₂COOH, —CH₂Cl, —CH₂CN, —CH₂OCH₃, —CH(CH₃)OCH₃,

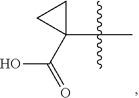 [SMILES: *C1(C(=O)O)CC1]

—C(O)OCH₂CH₃, —C(O)NH₂,

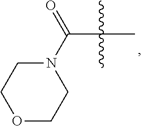 [SMILES: *C(=O)N1CCOCC1]

—CH₂—(OCH₂CH₂)₂OCH₃, —CH₂(OCH₂CH₂)₃OCH₃,

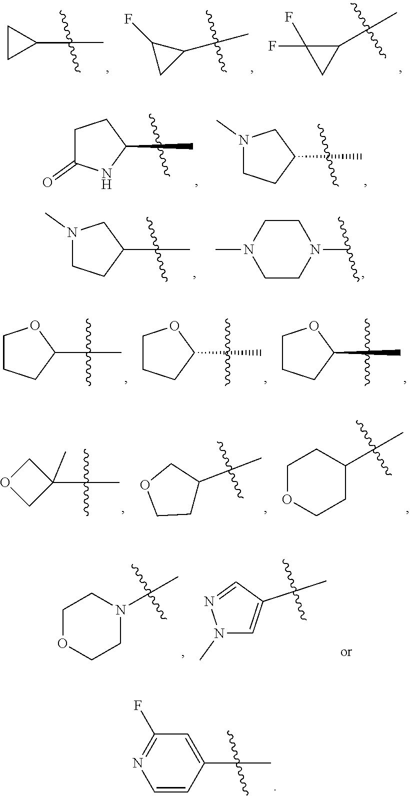

In one embodiment, the compound of formula (I) is a compound represented by formula (III):

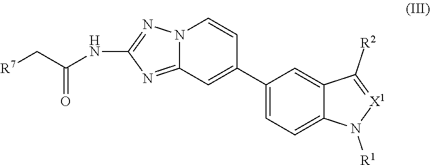

wherein X¹, R¹ and R² are each as defined for the compound of formula (I);
R⁷ is selected from —NR^{5a}R^{5b}, —(OCH₂CH₂)ₘOCH₃, —NH—C₁₋₆ alkyl-O—C₁₋₆ alkyl, -4- to 12-membered heterocycloalkyl, —NH-4- to 12-membered heterocycloalkyl, —NH—C₁₋₆ alkyl-4- to 12-membered heterocycloalkyl or 5- or 6-membered heteroaryl, wherein the said C₁₋₆ alkyl, C₃₋₆ cycloalkyl, 4- to 12-membered heterocycloalkyl and 5- or 6-membered heteroaryl are optionally substituted with one or more substituents selected from halogen, oxo, hydroxyl, carboxyl, cyano, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl or 4- to 12-membered heterocycloalkyl;
R^{5a} and R^{5b} are each independently selected from hydrogen, C₁₋₆ alkyl or C₁₋₆ alkoxy, wherein the said C₁₋₆ alkyl is optionally substituted with one or more halogen or C₃₋₆ cycloalkyl.

In one embodiment, R⁷ is selected from

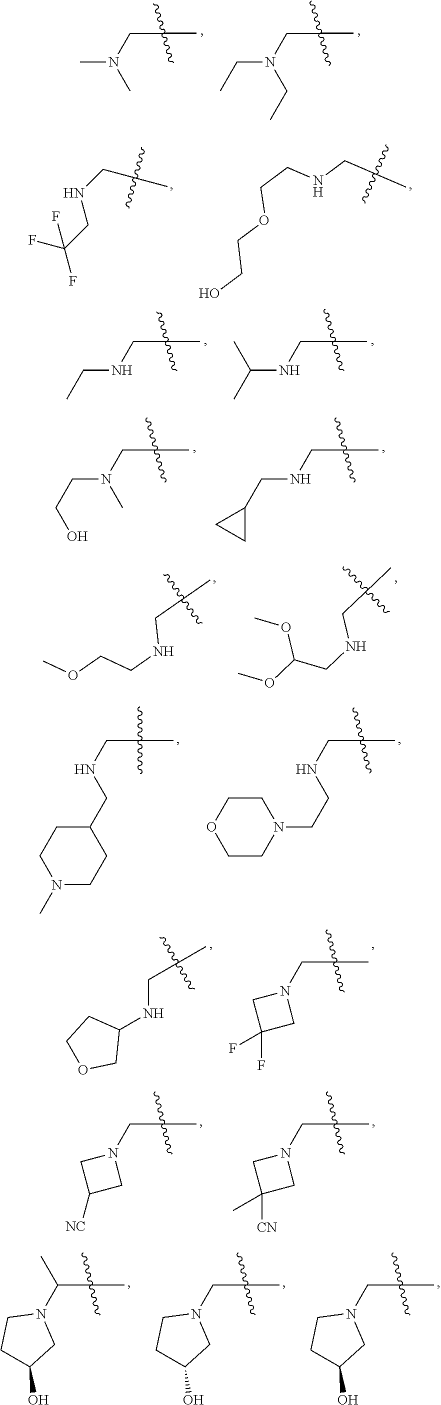

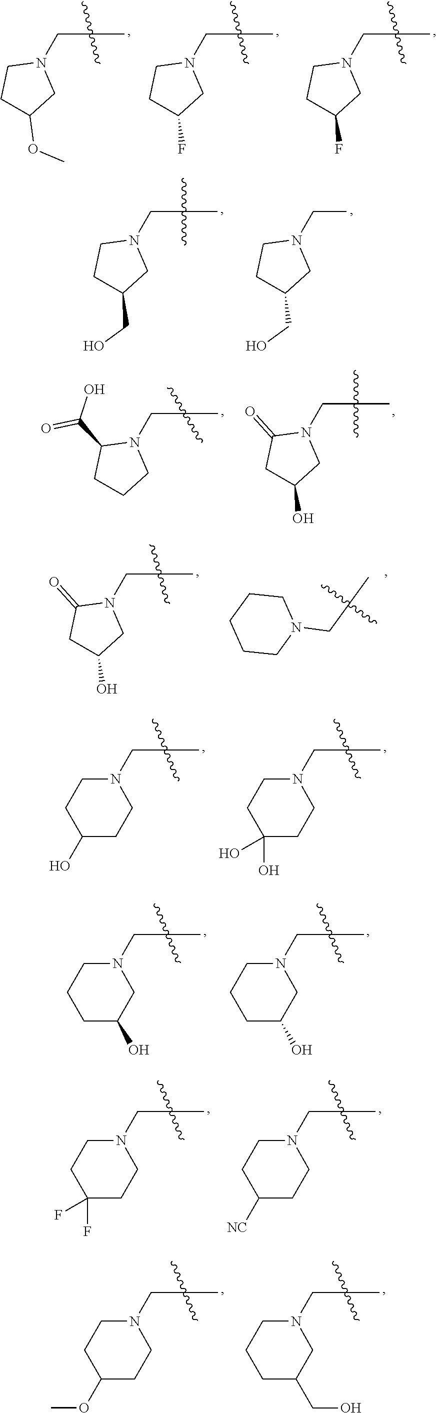

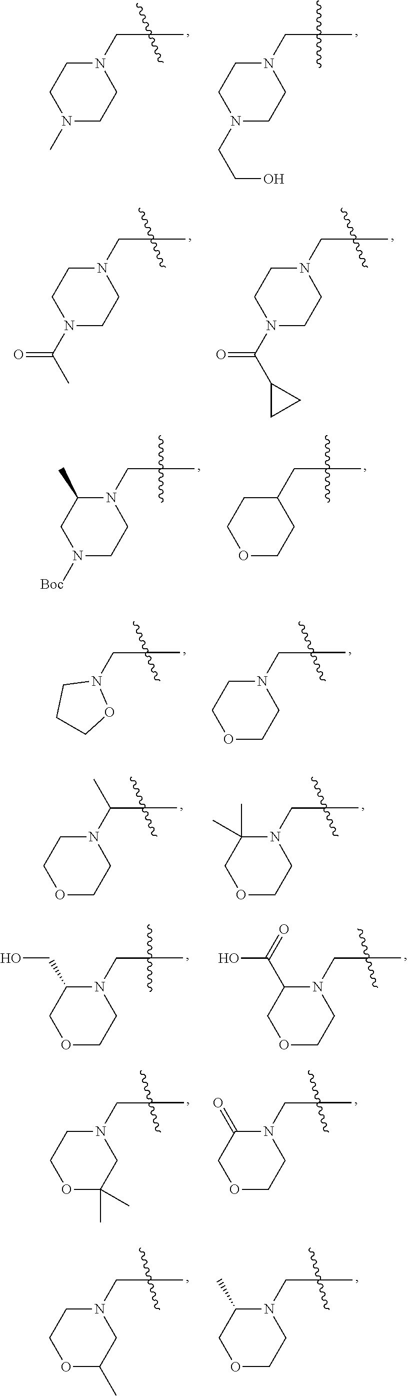

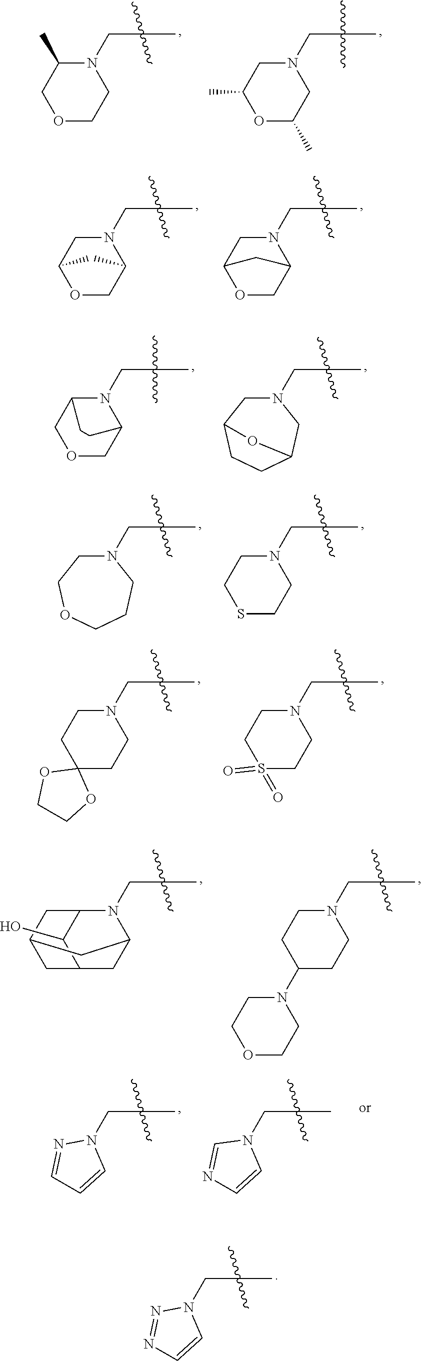

In one embodiment, the compound of formula (III) is a compound represented by formula (IIIa):

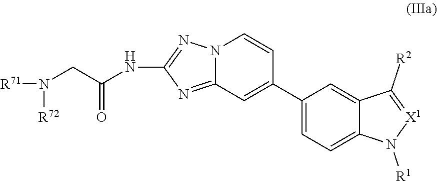

wherein X¹, R¹ and R² are each as defined for the compound of formula (I);
R⁷¹ and R⁷² are each independently selected from hydrogen C₁₋₆ alkyl, C₁₋₆ alkoxy, 4- to 12-membered heterocycloalkyl and 5- or 6-membered heteroaryl, wherein the said C₁₋₆ alkyl is optionally substituted with one or more substituents selected from halogen, C₁₋₆ alkoxy, C₃₋₆ cycloalkyl, 4- to 12-membered heterocycloalkyl or —CH(OCH₃)₂.

In one embodiment, the compound of formula (III) is a compound represented by formula (IIb):

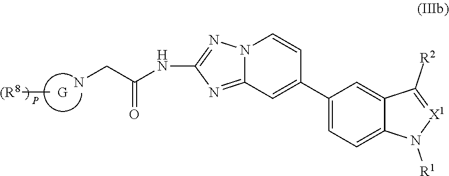

wherein X¹, R¹ and R² are each as defined for the compound of formula (I);
Ring G is selected from 5- or 6-membered nitrogen-containing heteroaryl or 5- to 12-membered nitrogen-containing heterocycloalkyl;
R⁸ is selected from hydrogen, halogen, cyano, hydroxyl, carboxyl, oxo, C₁₋₆ alkyl, C₁₋₆ alkoxy, —C₁₋₆ alkyl-OH, —C(O)—C₁₋₆ alkyl, —C(O)O—C₁₋₆ alkyl or —C(O)—C₃₋₆ cycloalkyl;
p is selected from 0, 1, 2 or 3, preferably 1 or 2;
preferably, R⁸ is selected from hydrogen, fluoro, cyano, hydroxyl, carboxyl, oxo, methyl, methoxy, hydroxymethyl, —C(O)CH₃, —C(O)-cyclopropyl or —C(O)OCH(CH₃)₃;

In one embodiment, ring G is selected from

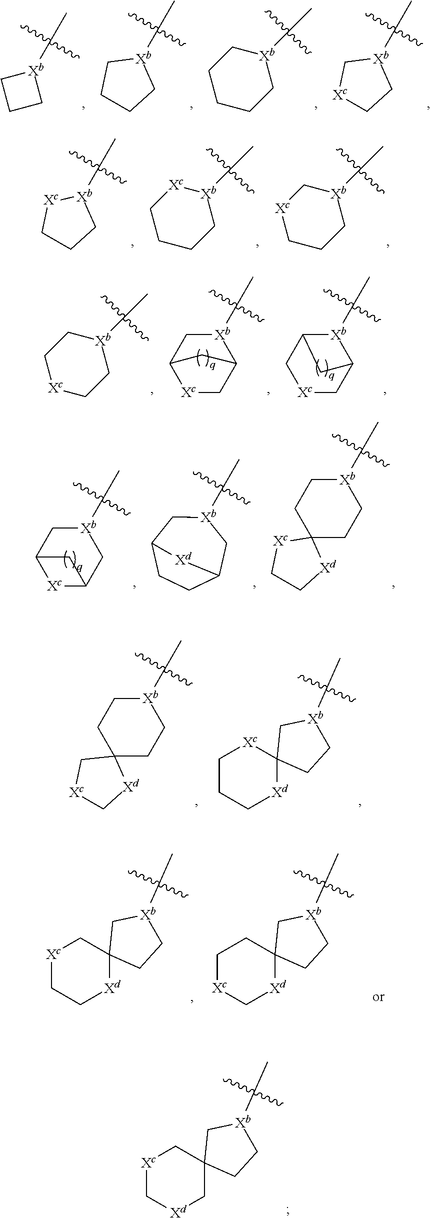

wherein X^{b} is N, and X^{c} and X^{d} are each independently selected from CH₂, NH, N, O or S;
q is selected from 1, 2 or 3, preferably 1 or 2;
preferably, ring G is selected from

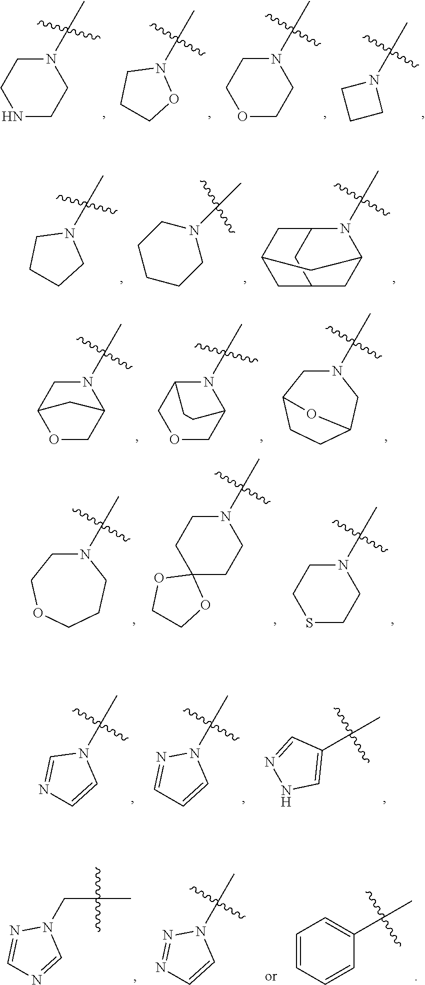

In one embodiment, the said 4- to 12-membered heterocycloalkyl is selected from 4- to 7-membered monocylic heterocycloalkyl, 6- to 8-membered bicyclic heterocycloalkyl, 6- to 10-membered bridged heterocycloalkyl, or 6- to 10-membered spiroheterocycloalkyl; the said 4- to 7-membered monocyclic heterocycloalkyl, 6- to 8-membered bicyclic heterocycloalkyl, 6- to 10-membered bridged heterocycloalkyl, or 6- to 10-membered spiroheterocycloalkyl preferably contains 1, 2, or 3 heteroatoms selected from N, S, or O as ring atoms.

In one embodiment, the said 4- to 12-membered heterocycloalkyl is selected from

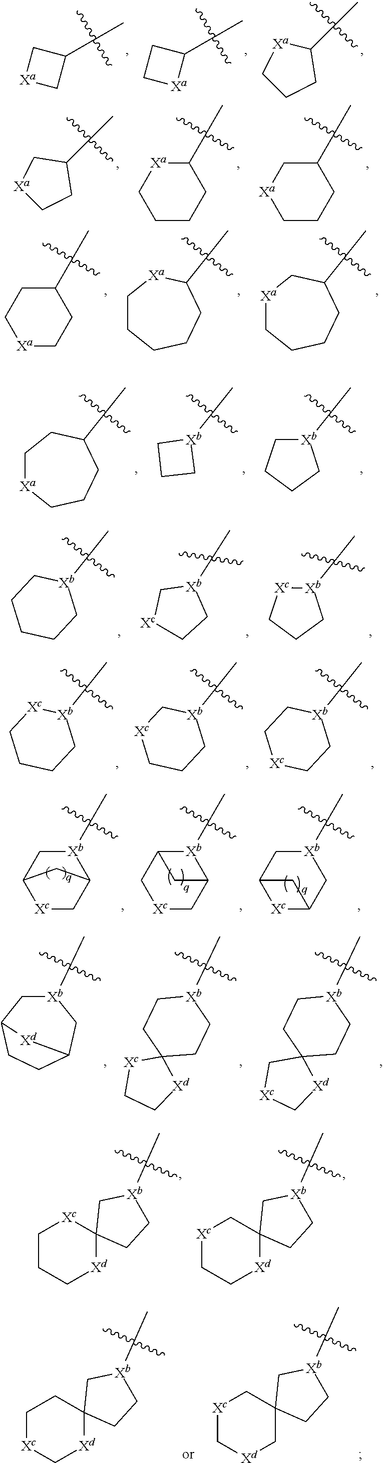

wherein X^{a}, X^{b}, X^{c}, and X^{d} are each independently selected from CH₂, NH, N, O, or S; q is selected from 1, 2, or 3, preferably 1 or 2;
preferably, X^{b} is N;
preferably, the said 4- to 12-membered heterocycloalkyl is selected from pyrrolidinyl, piperidinyl, piperazinyl, oxetanyl, tetrahydrofuranyl, oxetanyl, isoxazolidinyl, morpholinyl, oxazepanyl, azetidinyl, pyrrolidinyl, piperidinyl, azaadamantyl, oxa-azabicycloheptyl, oxa-azabicyclooctyl, oxa-azaspirodecyl or thiomorpholinyl;
preferably, the said 4- to 12-membered heterocycloalkyl is selected from

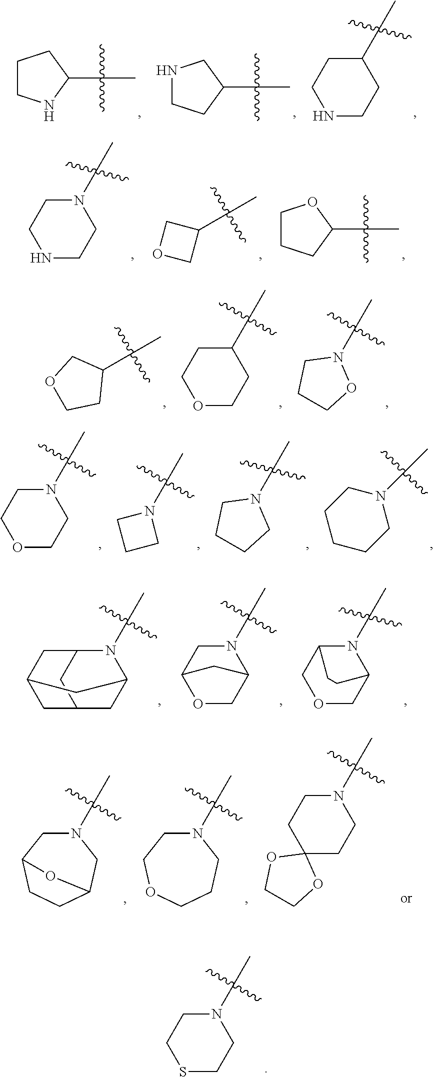

In one embodiment, the said 5- or 6-membered heteroaryl is selected from 5- or 6-membered nitrogen-containing heteroaryl, wherein the said 5- or 6-membered nitrogen-containing heteroaryl optionally contains 1, 2 or 3 nitrogen atoms as ring atoms; preferably, the said 5- or 6-membered heteroaryl is selected from imidazolyl, pyrazolyl, triazolyl or pyridyl;
preferably, the said 5- or 6-membered heteroaryl is selected from

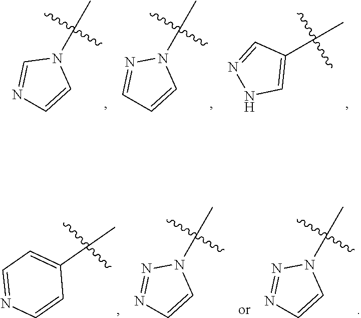 [SMILES: *n1ccnc1 ; *n1cccn1 ; *c1cn[nH]c1 ; *c1ccncc1 ; *n1ccnn1 ; *n1ccnn1]

The present invention provides the following compounds, or pharmaceutically acceptable salts, solvates or stereoisomers thereof:

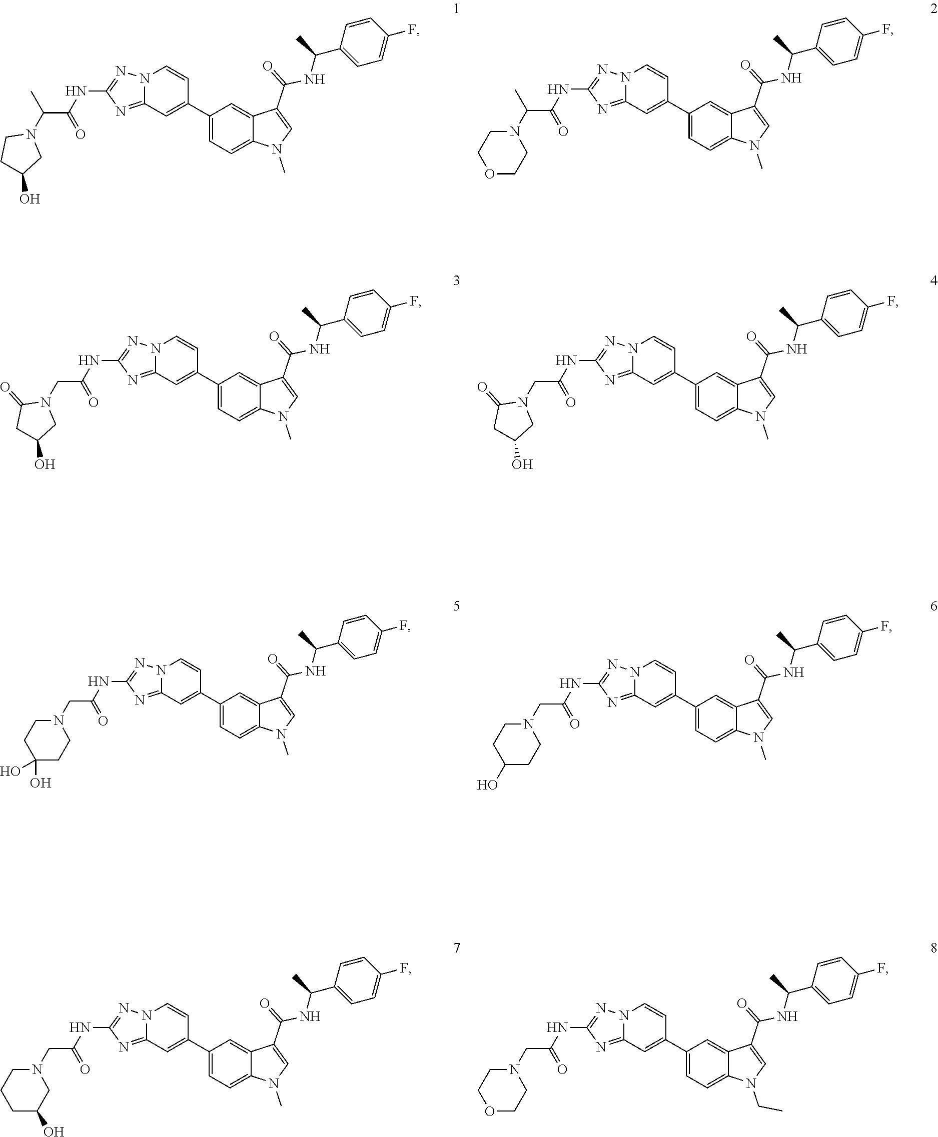

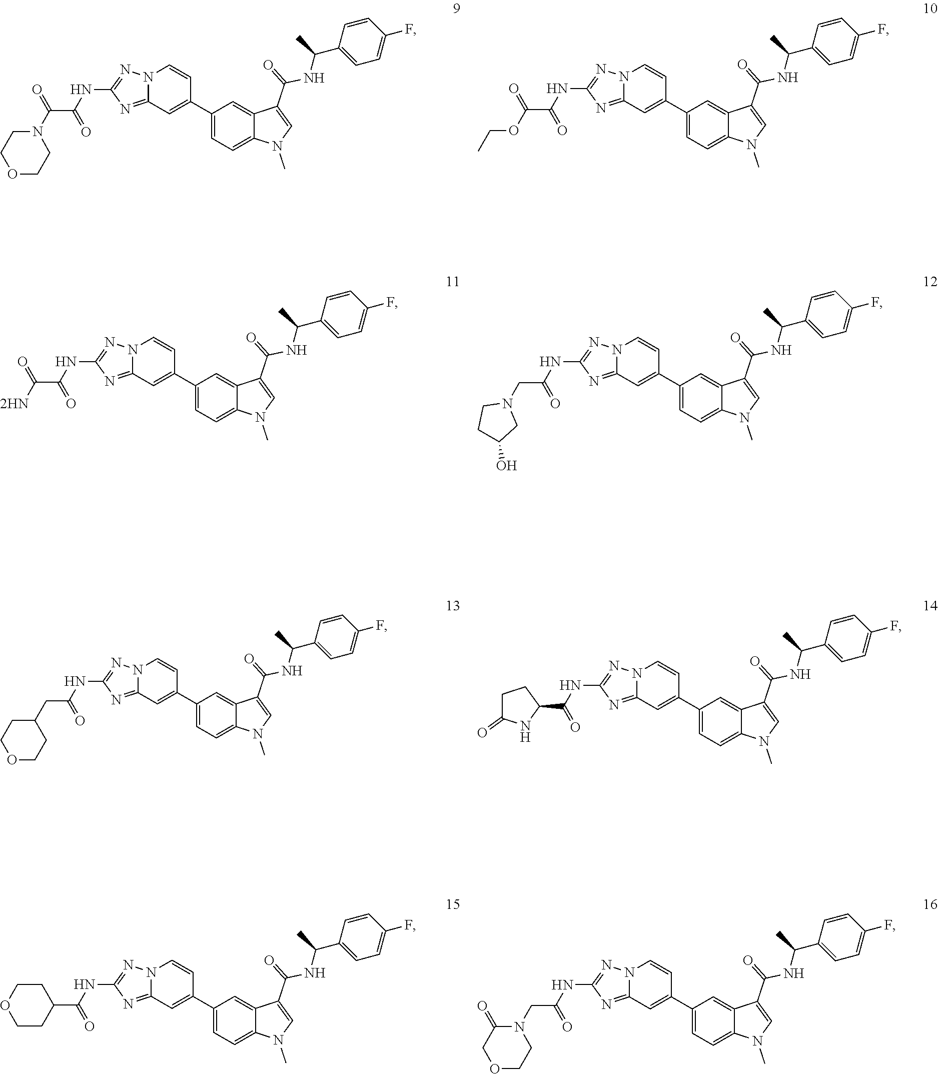

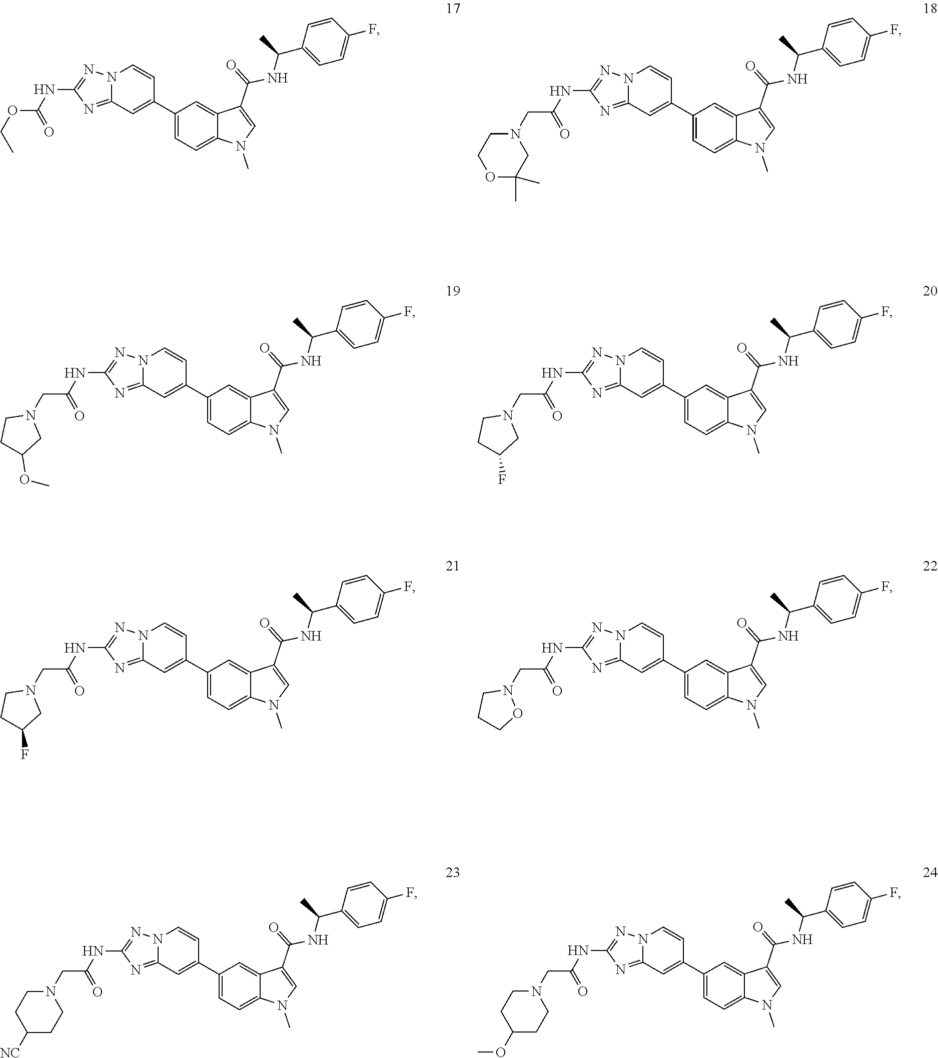

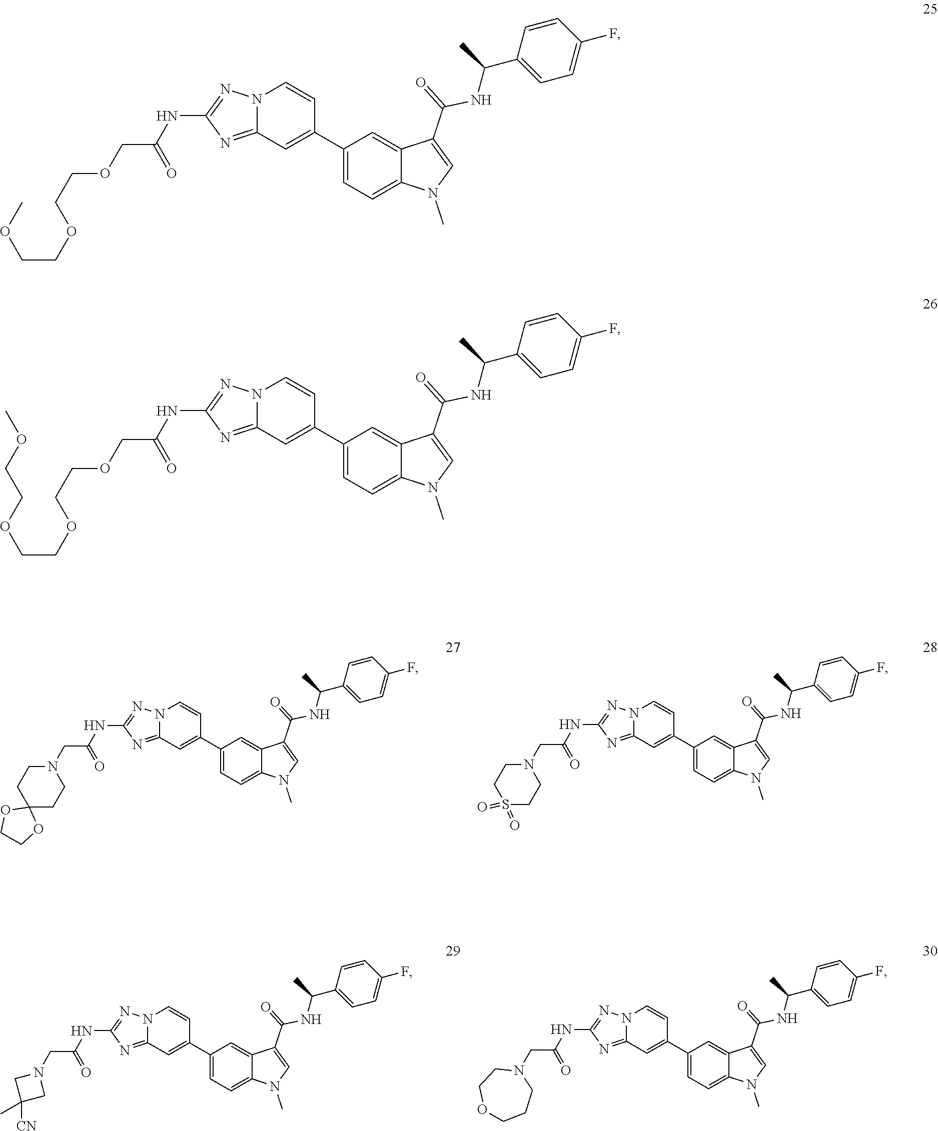 [SMILES: COCCOCCOCC(=O)Nc1nc2cc(-c3ccc4c(c3)c(C(=O)N[C@@H](C)c3ccc(F)cc3)cn4C)ccn2n1 ; COCCOCCOCCOCC(=O)Nc1nc2cc(-c3ccc4c(c3)c(C(=O)N[C@@H](C)c3ccc(F)cc3)cn4C)ccn2n1 ; C[C@H](NC(=O)c1cn(C)c2ccc(-c3ccn4nc(NC(=O)CN5CCC6(CC5)OCCO6)nc4c3)cc12)c1ccc(F)cc1 ; C[C@H](NC(=O)c1cn(C)c2ccc(-c3ccn4nc(NC(=O)CN5CCS(=O)(=O)CC5)nc4c3)cc12)c1ccc(F)cc1 ; C[C@H](NC(=O)c1cn(C)c2ccc(-c3ccn4nc(NC(=O)CN5CC(C)(C#N)C5)nc4c3)cc12)c1ccc(F)cc1 ; C[C@H](NC(=O)c1cn(C)c2ccc(-c3ccn4nc(NC(=O)CN5CCCOCC5)nc4c3)cc12)c1ccc(F)cc1]

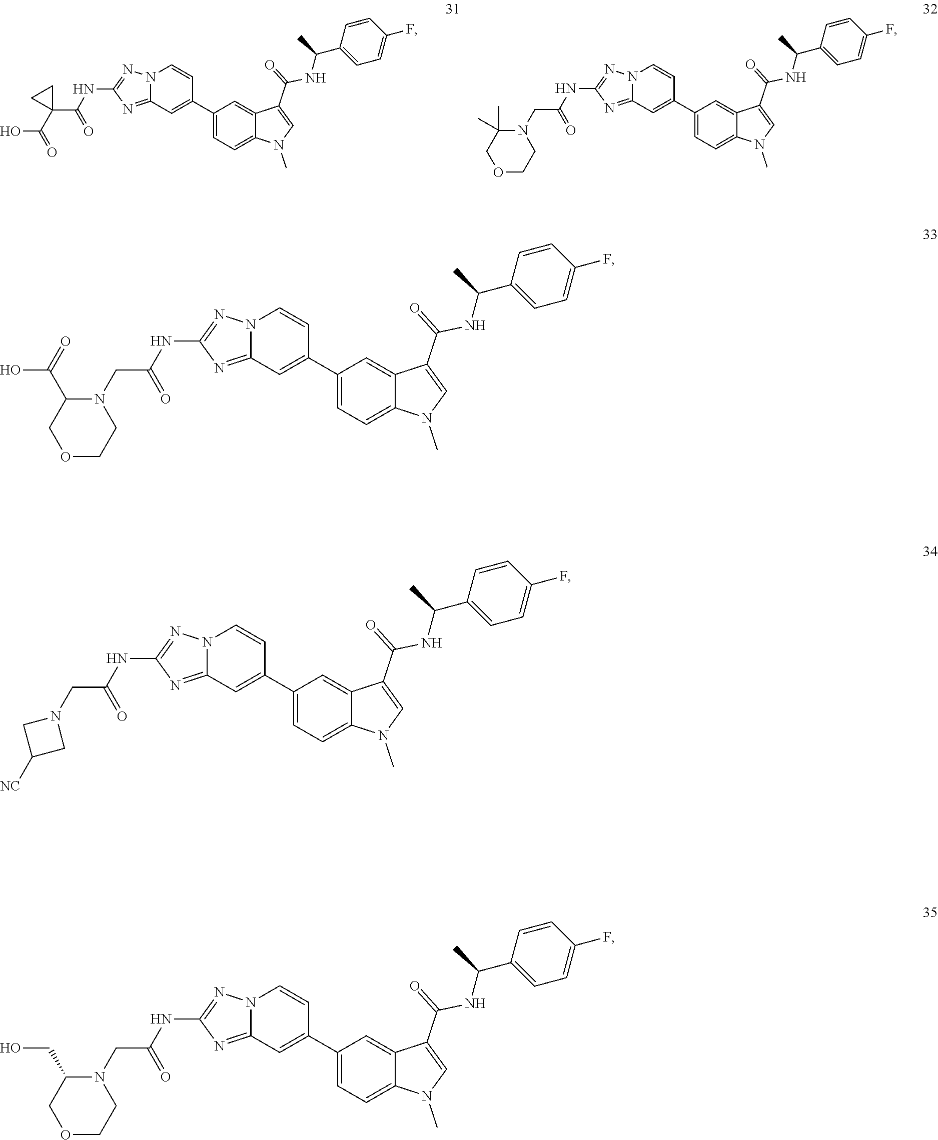 [SMILES: C[C@H](NC(=O)c1cn(C)c2ccc(-c3ccn4nc(NC(=O)C5(C(=O)O)CC5)nc4c3)cc12)c1ccc(F)cc1 ; C[C@H](NC(=O)c1cn(C)c2ccc(-c3ccn4nc(NC(=O)CN5CCOCC5(C)C)nc4c3)cc12)c1ccc(F)cc1 ; C[C@H](NC(=O)c1cn(C)c2ccc(-c3ccn4nc(NC(=O)CN5CCOCC5C(=O)O)nc4c3)cc12)c1ccc(F)cc1 ; C[C@H](NC(=O)c1cn(C)c2ccc(-c3ccn4nc(NC(=O)CN5CC(C#N)C5)nc4c3)cc12)c1ccc(F)cc1 ; C[C@H](NC(=O)c1cn(C)c2ccc(-c3ccn4nc(NC(=O)CN5CCOC[C@@H]5CO)nc4c3)cc12)c1ccc(F)cc1]

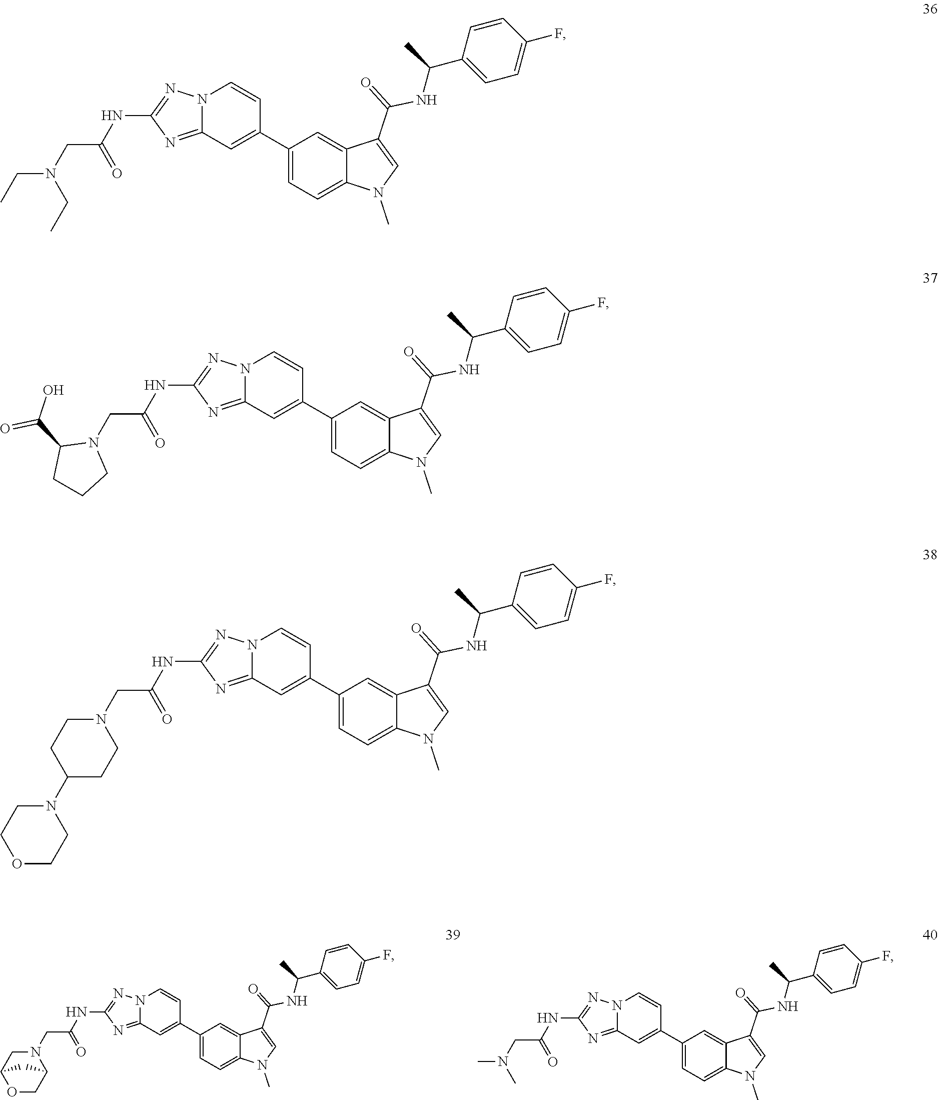 [SMILES: CCN(CC)CC(=O)Nc1nc2cc(-c3ccc4c(c3)c(C(=O)N[C@@H](C)c3ccc(F)cc3)cn4C)ccn2n1 ; C[C@H](NC(=O)c1cn(C)c2ccc(-c3ccn4nc(NC(=O)CN5CCC[C@H]5C(=O)O)nc4c3)cc12)c1ccc(F)cc1 ; C[C@H](NC(=O)c1cn(C)c2ccc(-c3ccn4nc(NC(=O)CN5CCC(N6CCOCC6)CC5)nc4c3)cc12)c1ccc(F)cc1 ; C[C@H](NC(=O)c1cn(C)c2ccc(-c3ccn4nc(NC(=O)CN5C[C@H]6C[C@@H]5CO6)nc4c3)cc12)c1ccc(F)cc1 ; C[C@H](NC(=O)c1cn(C)c2ccc(-c3ccn4nc(NC(=O)CN(C)C)nc4c3)cc12)c1ccc(F)cc1]

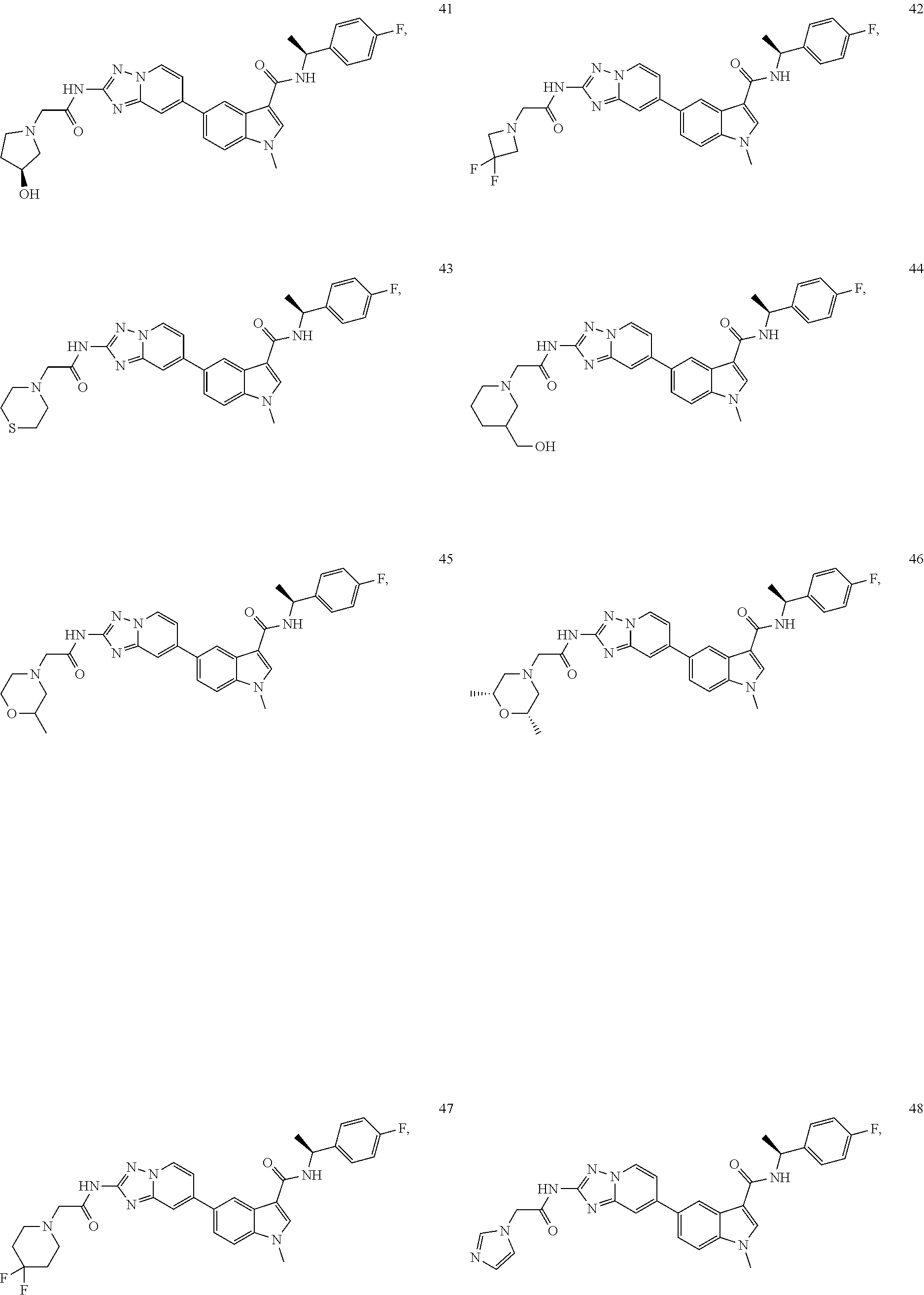

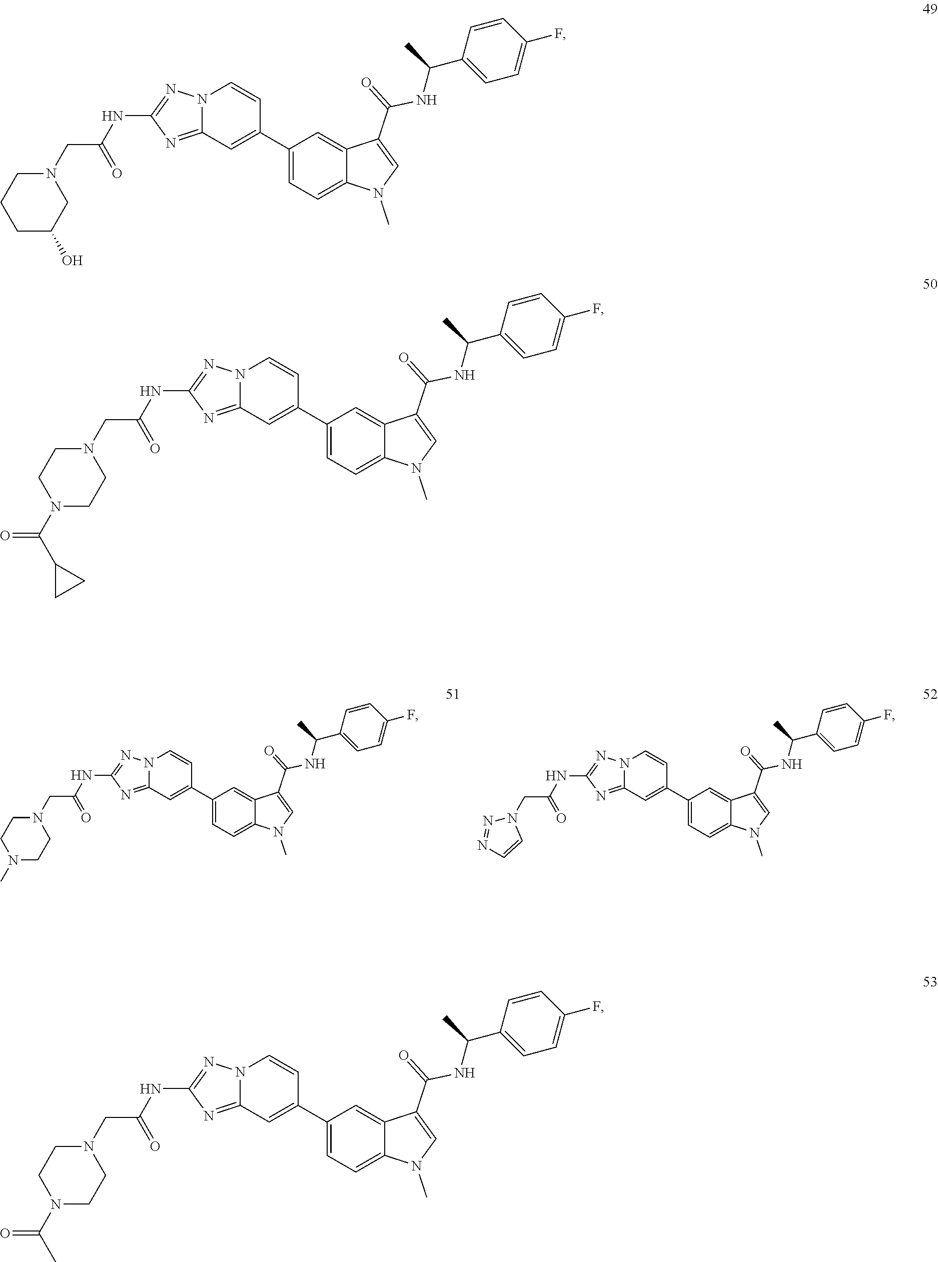 [SMILES: C[C@H](NC(=O)c1cn(C)c2ccc(-c3ccn4nc(NC(=O)CN5CCC[C@@H](O)C5)nc4c3)cc12)c1ccc(F)cc1 ; C[C@H](NC(=O)c1cn(C)c2ccc(-c3ccn4nc(NC(=O)CN5CCN(C(=O)C6CC6)CC5)nc4c3)cc12)c1ccc(F)cc1 ; C[C@H](NC(=O)c1cn(C)c2ccc(-c3ccn4nc(NC(=O)CN5CCN(C)CC5)nc4c3)cc12)c1ccc(F)cc1 ; C[C@H](NC(=O)c1cn(C)c2ccc(-c3ccn4nc(NC(=O)Cn5ccnn5)nc4c3)cc12)c1ccc(F)cc1 ; CC(=O)N1CCN(CC(=O)Nc2nc3cc(-c4ccc5c(c4)c(C(=O)N[C@@H](C)c4ccc(F)cc4)cn5C)ccn3n2)CC1]

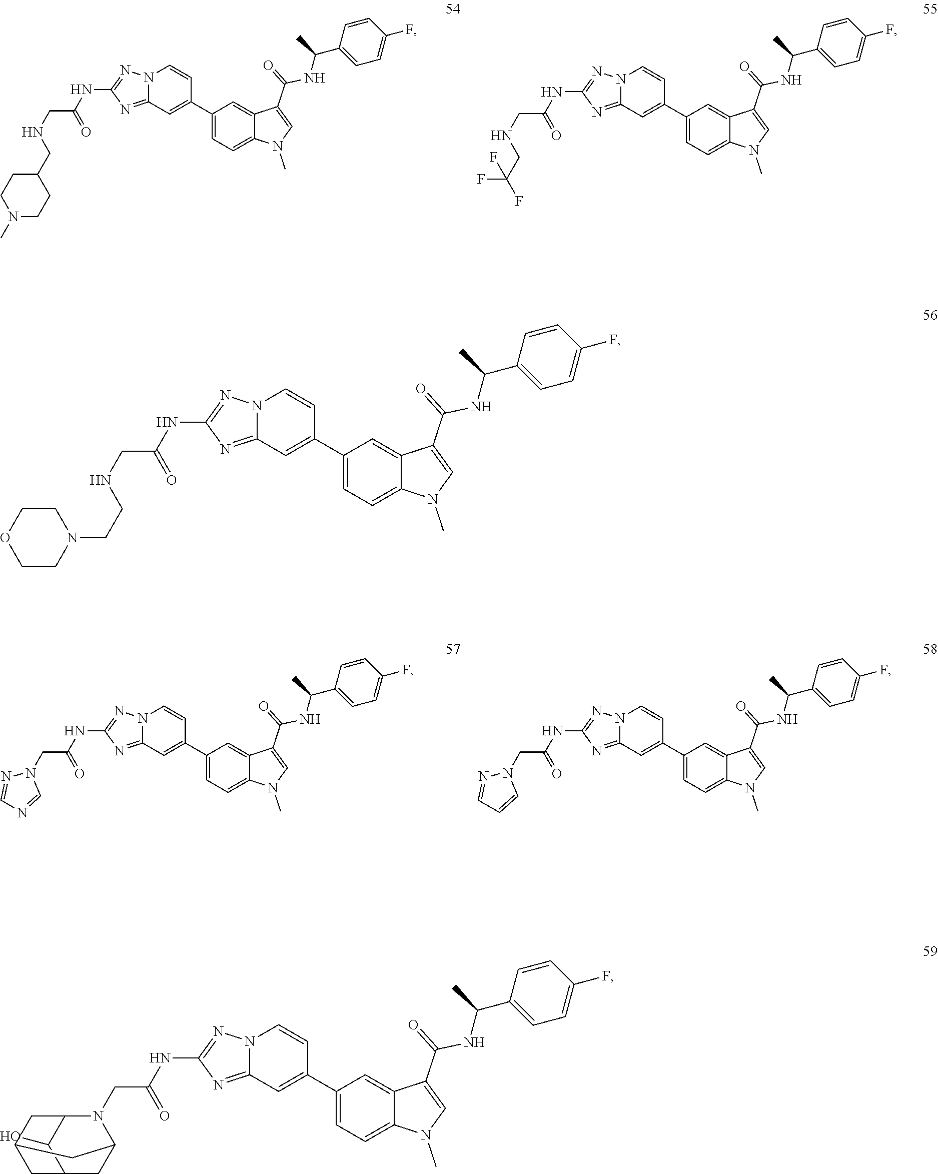 [SMILES: C[C@H](NC(=O)c1cn(C)c2ccc(-c3ccn4nc(NC(=O)CNCC5CCN(C)CC5)nc4c3)cc12)c1ccc(F)cc1 ; C[C@H](NC(=O)c1cn(C)c2ccc(-c3ccn4nc(NC(=O)CNCC(F)(F)F)nc4c3)cc12)c1ccc(F)cc1 ; C[C@H](NC(=O)c1cn(C)c2ccc(-c3ccn4nc(NC(=O)CNCCN5CCOCC5)nc4c3)cc12)c1ccc(F)cc1 ; C[C@H](NC(=O)c1cn(C)c2ccc(-c3ccn4nc(NC(=O)Cn5cncn5)nc4c3)cc12)c1ccc(F)cc1 ; C[C@H](NC(=O)c1cn(C)c2ccc(-c3ccn4nc(NC(=O)Cn5cccn5)nc4c3)cc12)c1ccc(F)cc1 ; C[C@H](NC(=O)c1cn(C)c2ccc(-c3ccn4nc(NC(=O)CN5C6CC7CC(C6)C(O)C5C7)nc4c3)cc12)c1ccc(F)cc1]

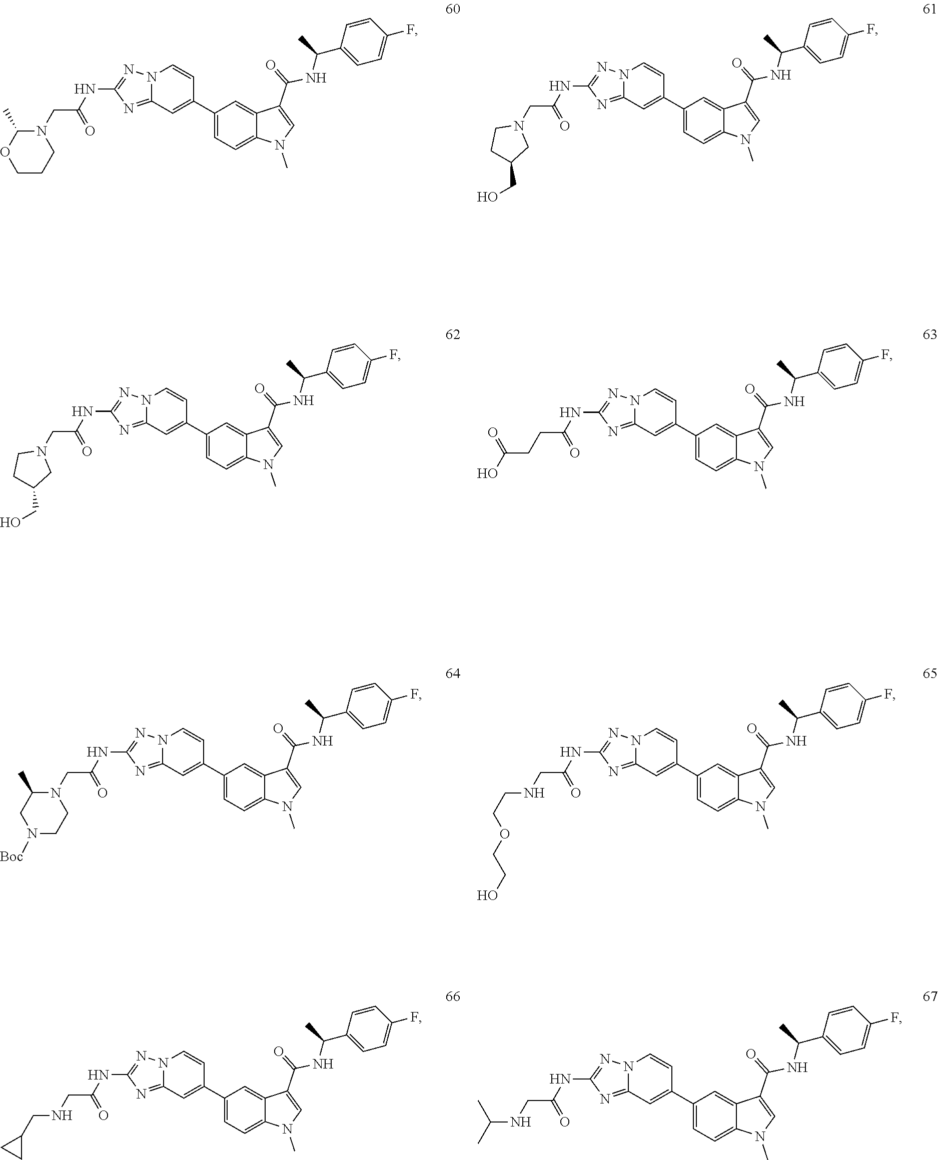

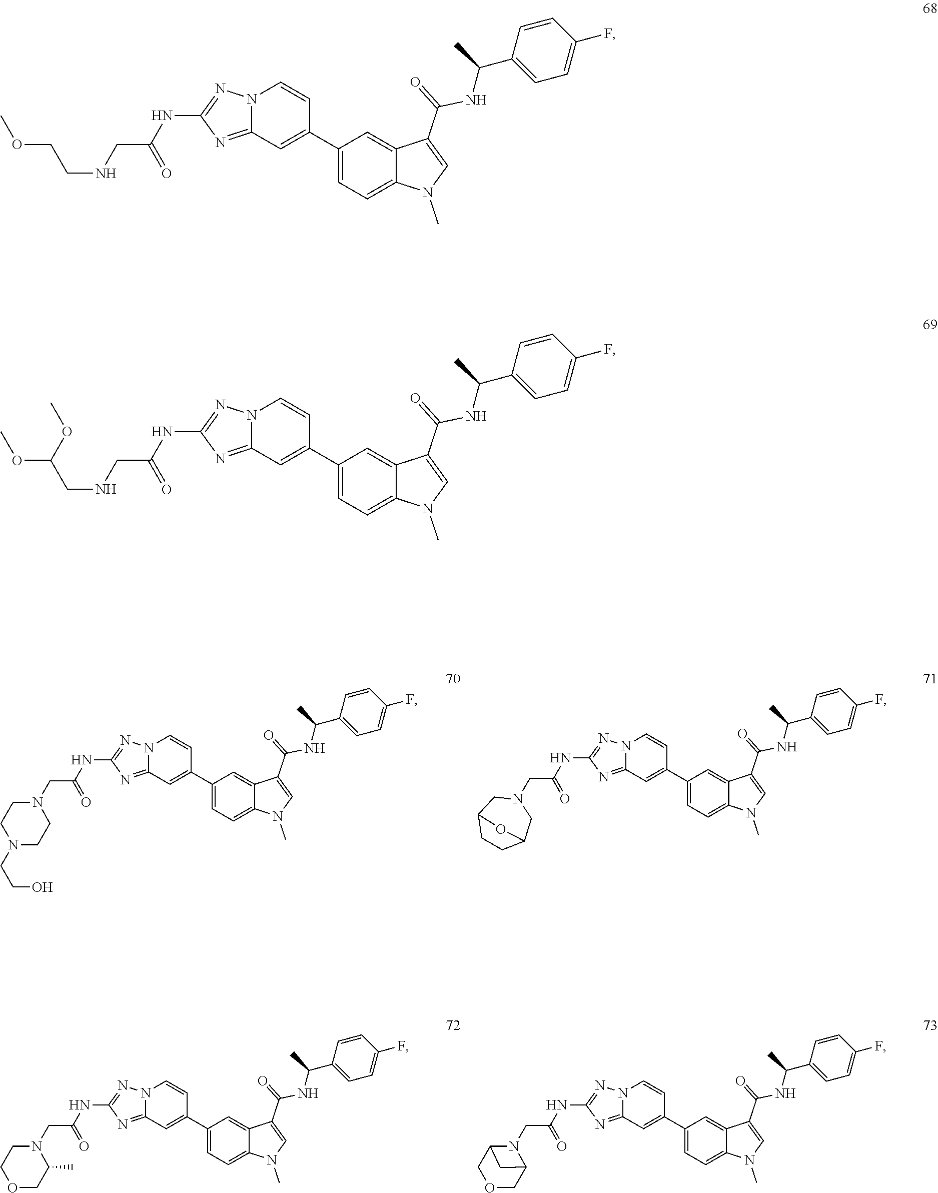 [SMILES: COCCNCC(=O)Nc1nc2cc(-c3ccc4c(c3)c(C(=O)N[C@@H](C)c3ccc(F)cc3)cn4C)ccn2n1 ; COC(CNCC(=O)Nc1nc2cc(-c3ccc4c(c3)c(C(=O)N[C@@H](C)c3ccc(F)cc3)cn4C)ccn2n1)OC ; C[C@H](NC(=O)c1cn(C)c2ccc(-c3ccn4nc(NC(=O)CN5CCN(CCO)CC5)nc4c3)cc12)c1ccc(F)cc1 ; C[C@H](NC(=O)c1cn(C)c2ccc(-c3ccn4nc(NC(=O)CN5CC6CCC(C5)O6)nc4c3)cc12)c1ccc(F)cc1 ; C[C@H](NC(=O)c1cn(C)c2ccc(-c3ccn4nc(NC(=O)CN5CCOC[C@H]5C)nc4c3)cc12)c1ccc(F)cc1 ; C[C@H](NC(=O)c1cn(C)c2ccc(-c3ccn4nc(NC(=O)CN5C6COCC5C6)nc4c3)cc12)c1ccc(F)cc1]

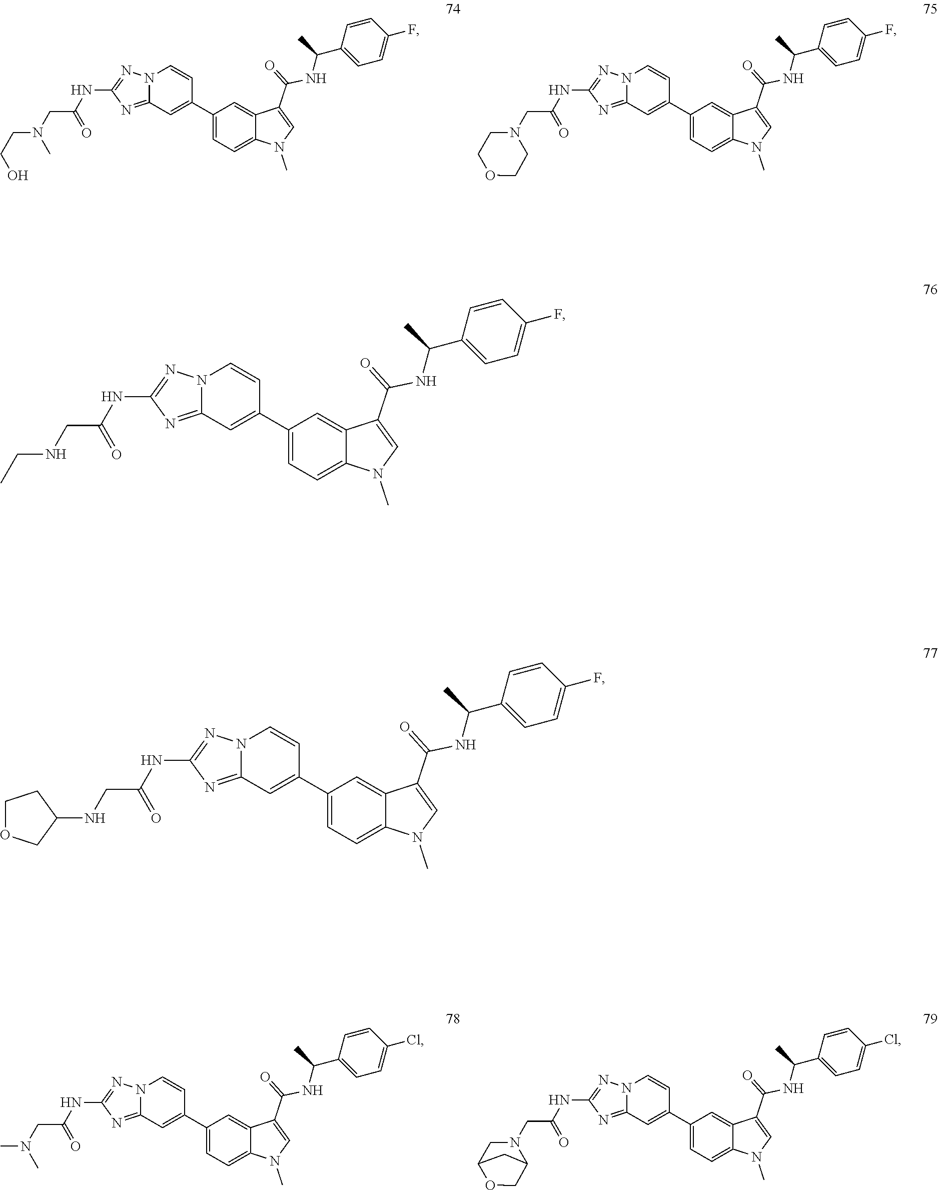 [SMILES: C[C@H](NC(=O)c1cn(C)c2ccc(-c3ccn4nc(NC(=O)CN(C)CCO)nc4c3)cc12)c1ccc(F)cc1 ; C[C@H](NC(=O)c1cn(C)c2ccc(-c3ccn4nc(NC(=O)CN5CCOCC5)nc4c3)cc12)c1ccc(F)cc1 ; CCNCC(=O)Nc1nc2cc(-c3ccc4c(c3)c(C(=O)N[C@@H](C)c3ccc(F)cc3)cn4C)ccn2n1 ; C[C@H](NC(=O)c1cn(C)c2ccc(-c3ccn4nc(NC(=O)CNC5CCOC5)nc4c3)cc12)c1ccc(F)cc1 ; C[C@H](NC(=O)c1cn(C)c2ccc(-c3ccn4nc(NC(=O)CN(C)C)nc4c3)cc12)c1ccc(Cl)cc1 ; C[C@H](NC(=O)c1cn(C)c2ccc(-c3ccn4nc(NC(=O)CN5CC6CC5CO6)nc4c3)cc12)c1ccc(Cl)cc1]

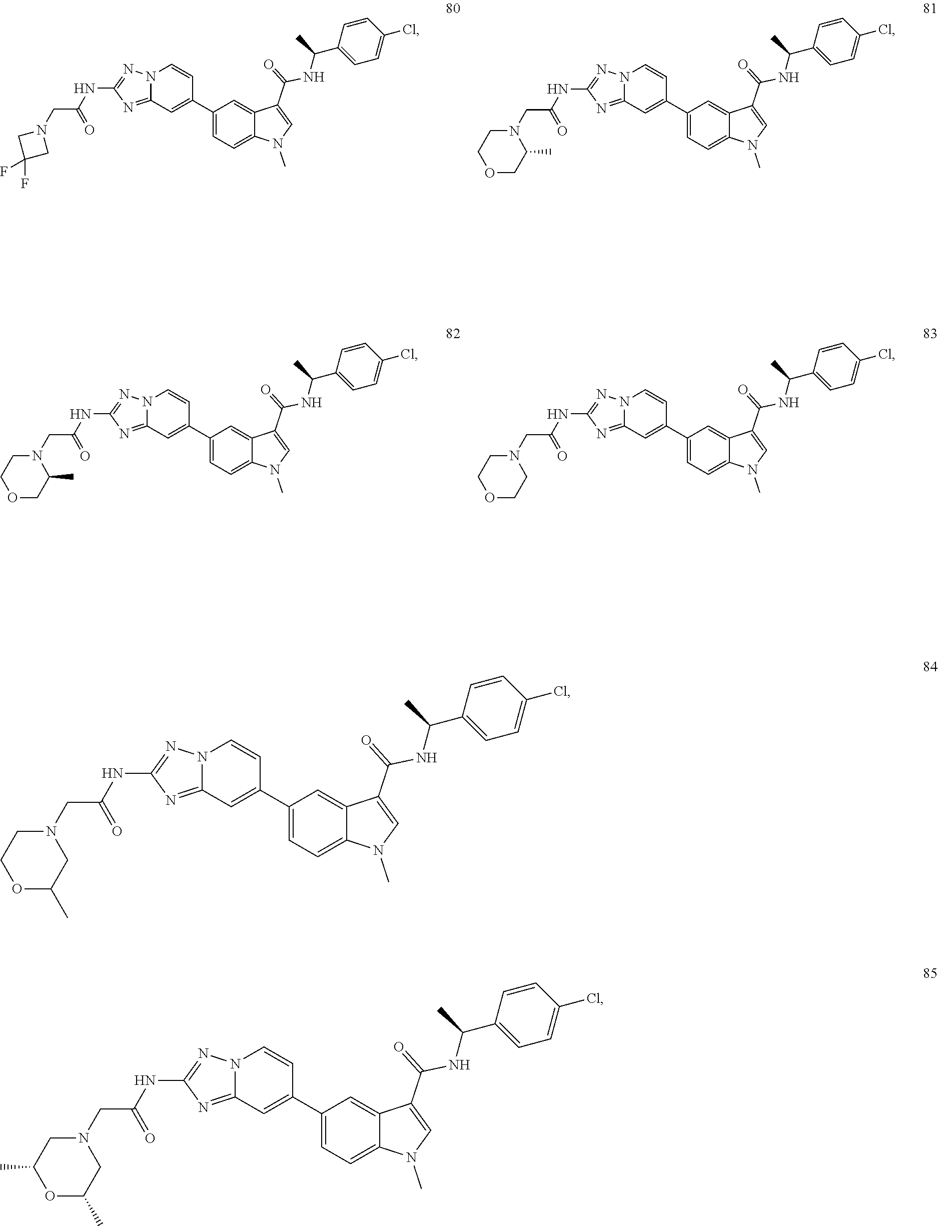 [SMILES: C[C@H](NC(=O)c1cn(C)c2ccc(-c3ccn4nc(NC(=O)CN5CC(F)(F)C5)nc4c3)cc12)c1ccc(Cl)cc1 ; C[C@H](NC(=O)c1cn(C)c2ccc(-c3ccn4nc(NC(=O)CN5CCOC[C@H]5C)nc4c3)cc12)c1ccc(Cl)cc1 ; C[C@H](NC(=O)c1cn(C)c2ccc(-c3ccn4nc(NC(=O)CN5CCOC[C@@H]5C)nc4c3)cc12)c1ccc(Cl)cc1 ; C[C@H](NC(=O)c1cn(C)c2ccc(-c3ccn4nc(NC(=O)CN5CCOCC5)nc4c3)cc12)c1ccc(Cl)cc1 ; CC1CN(CC(=O)Nc2nc3cc(-c4ccc5c(c4)c(C(=O)N[C@@H](C)c4ccc(Cl)cc4)cn5C)ccn3n2)CCO1 ; C[C@@H]1CN(CC(=O)Nc2nc3cc(-c4ccc5c(c4)c(C(=O)N[C@@H](C)c4ccc(Cl)cc4)cn5C)ccn3n2)C[C@H](C)O1]

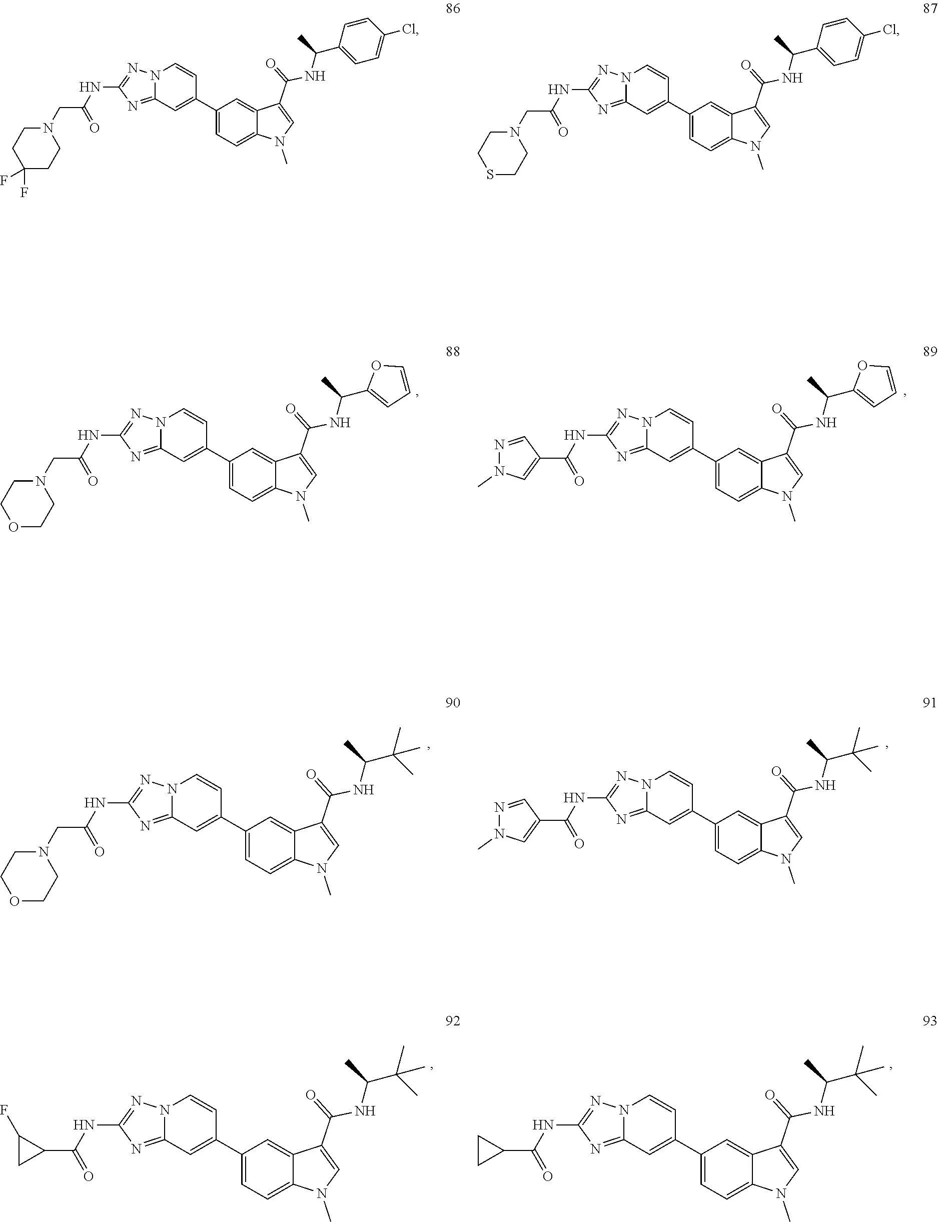

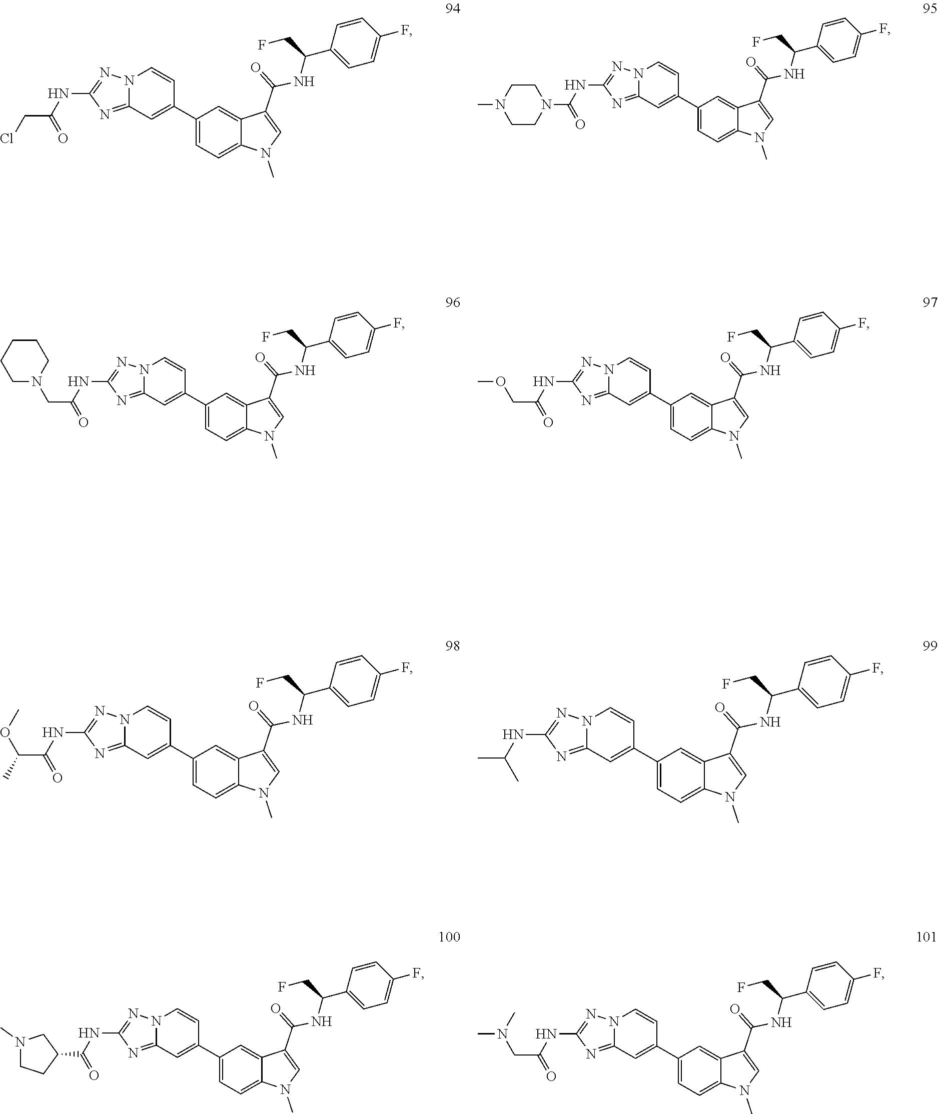

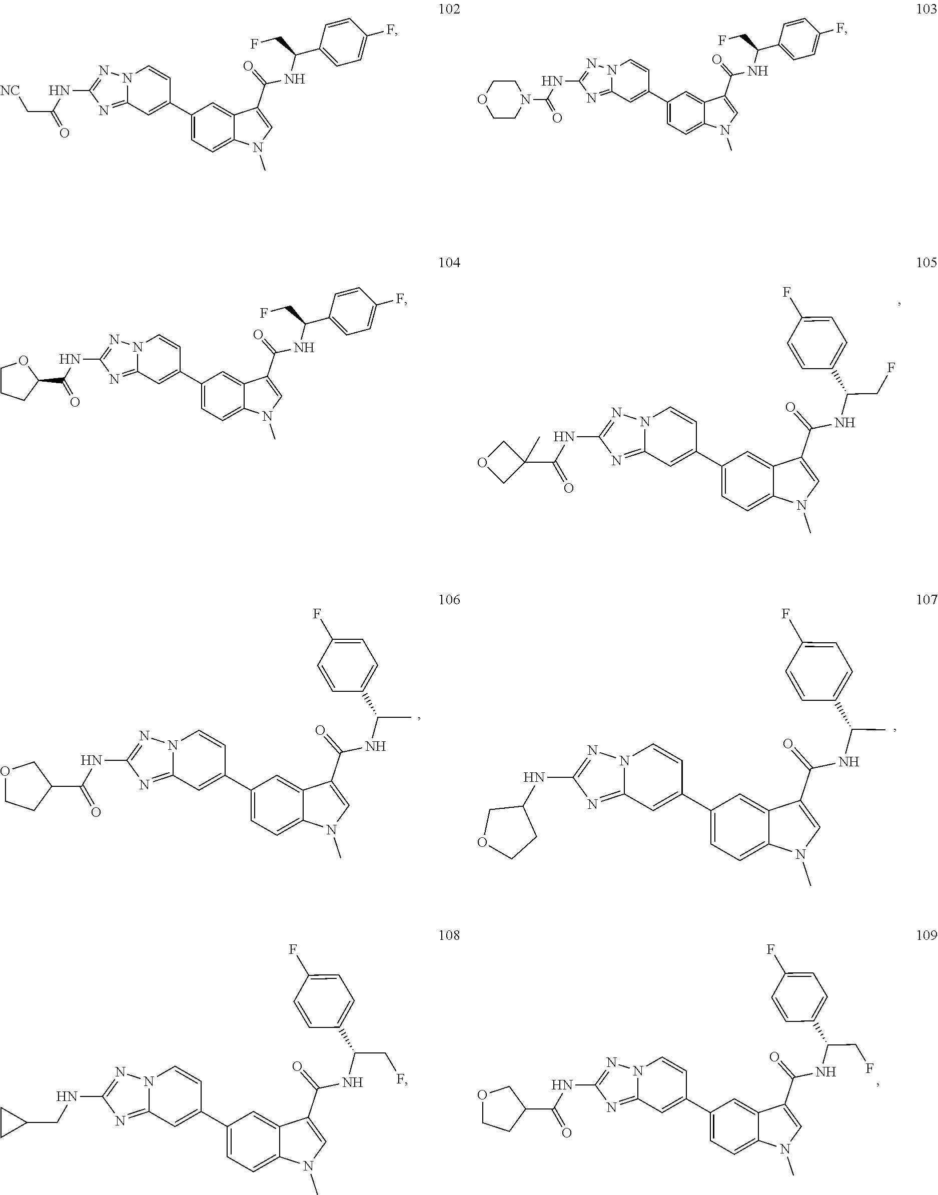

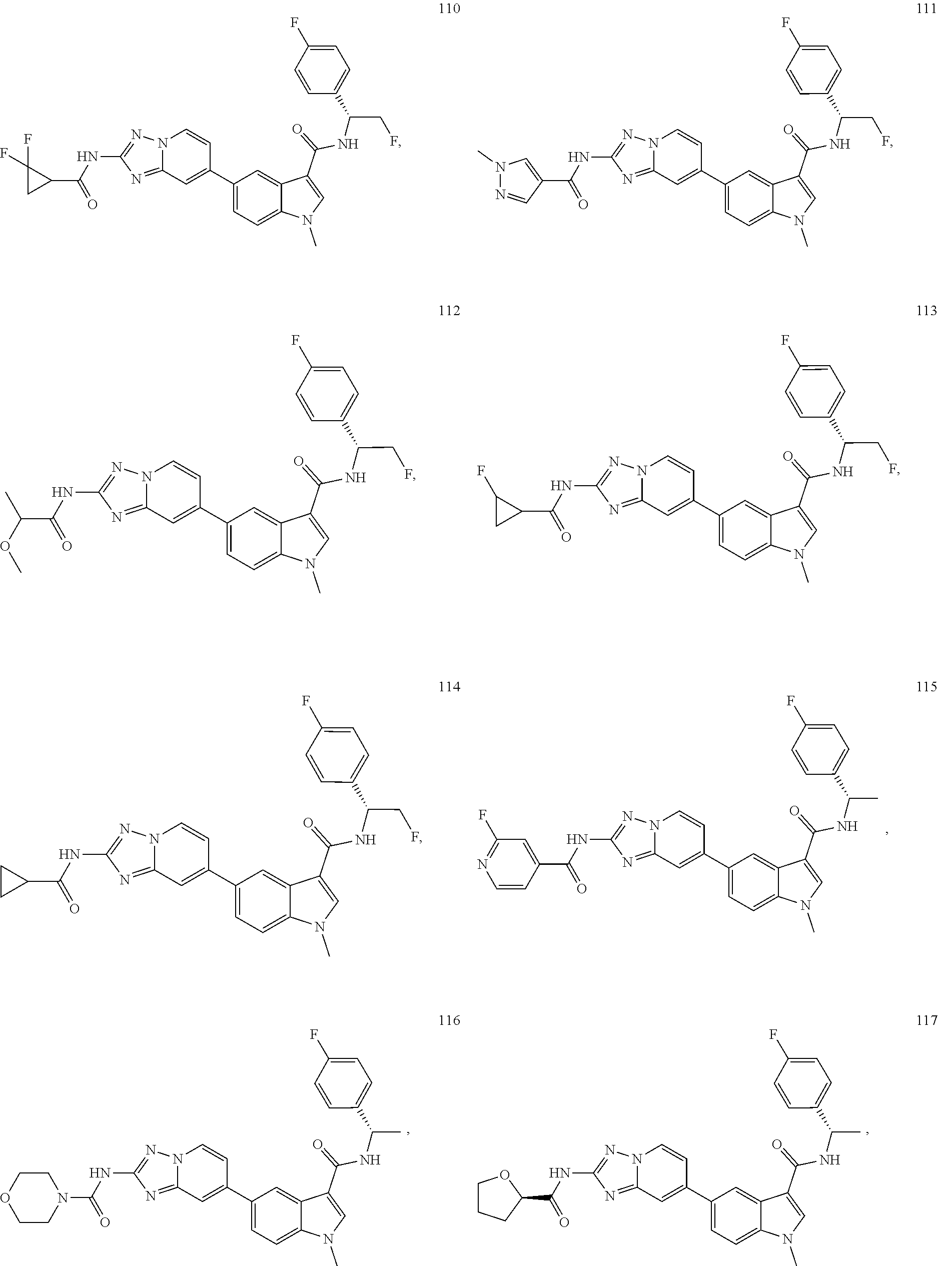

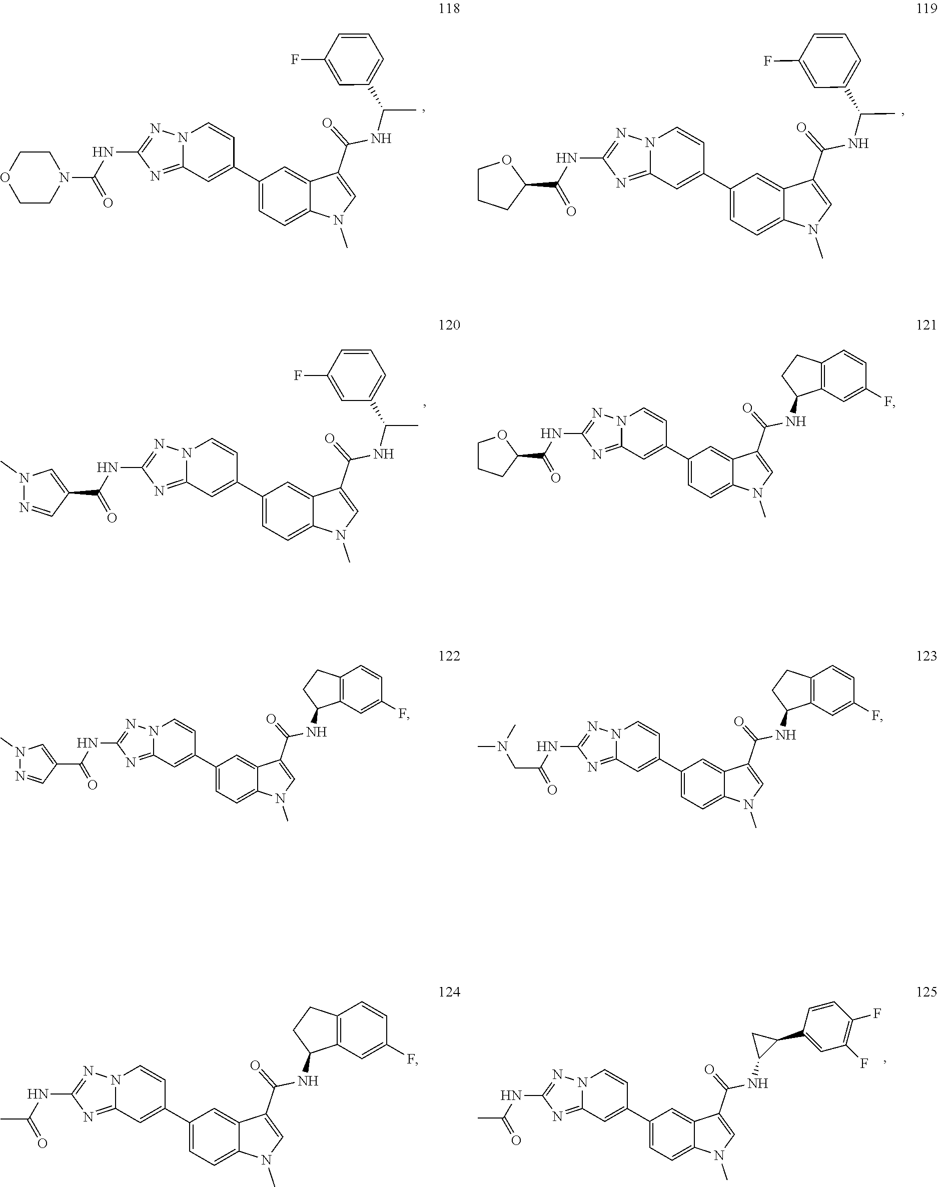

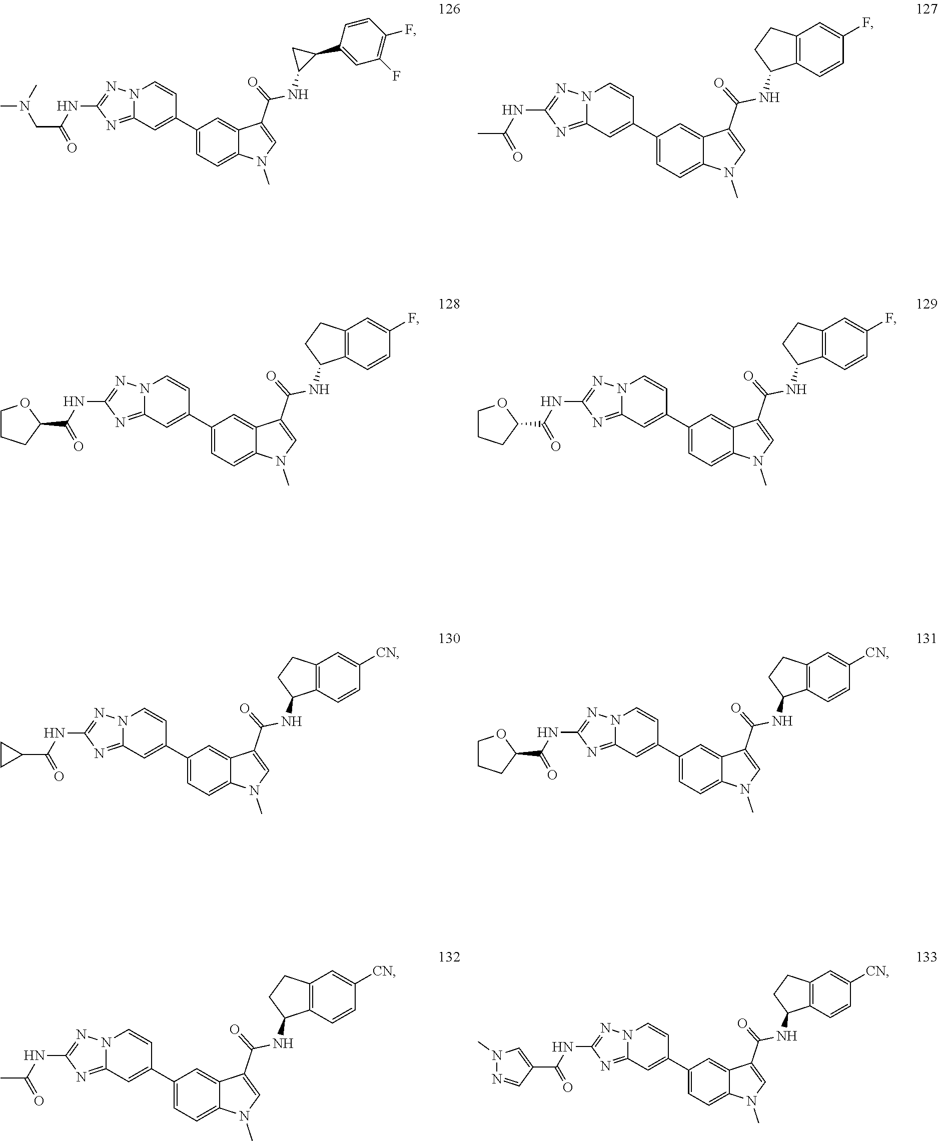

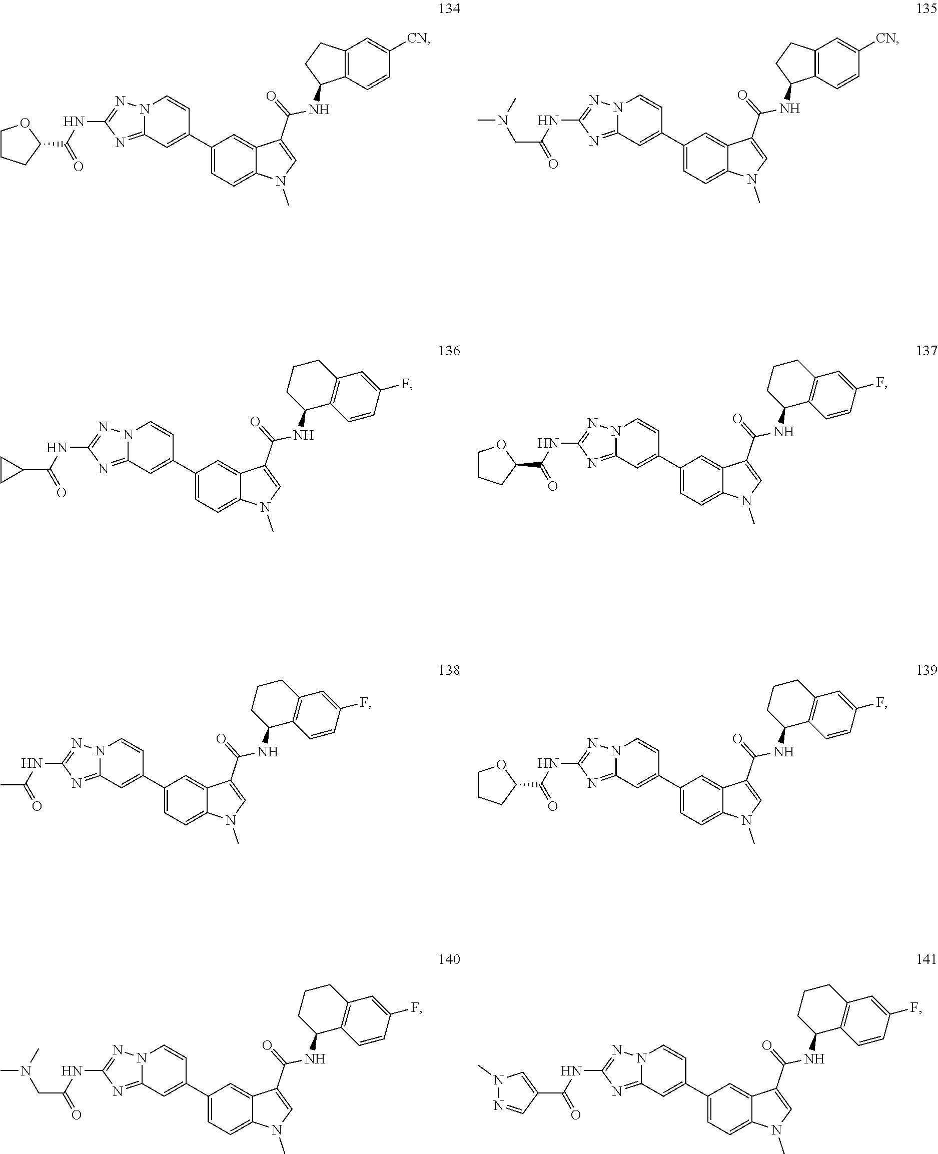

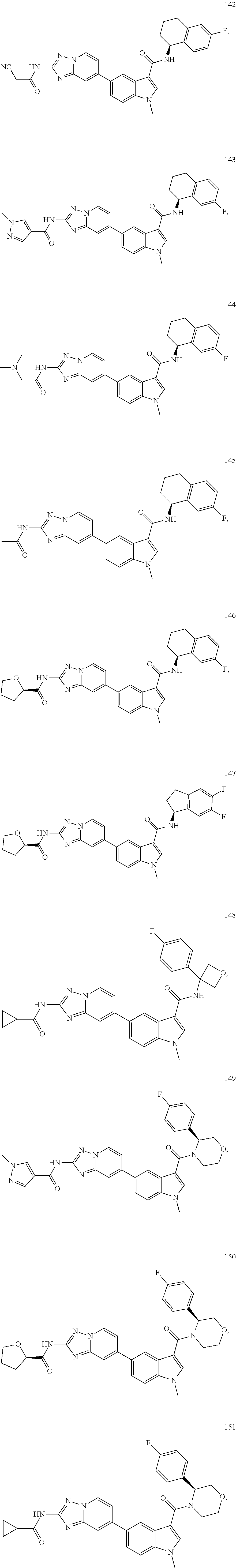

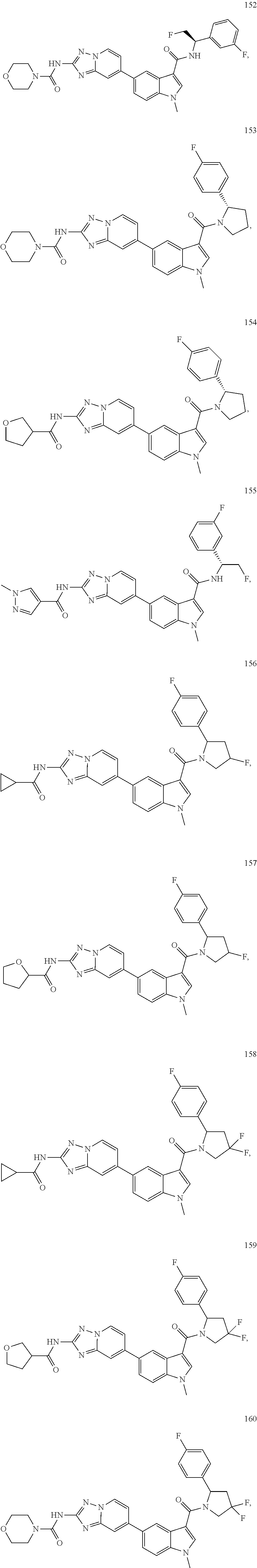

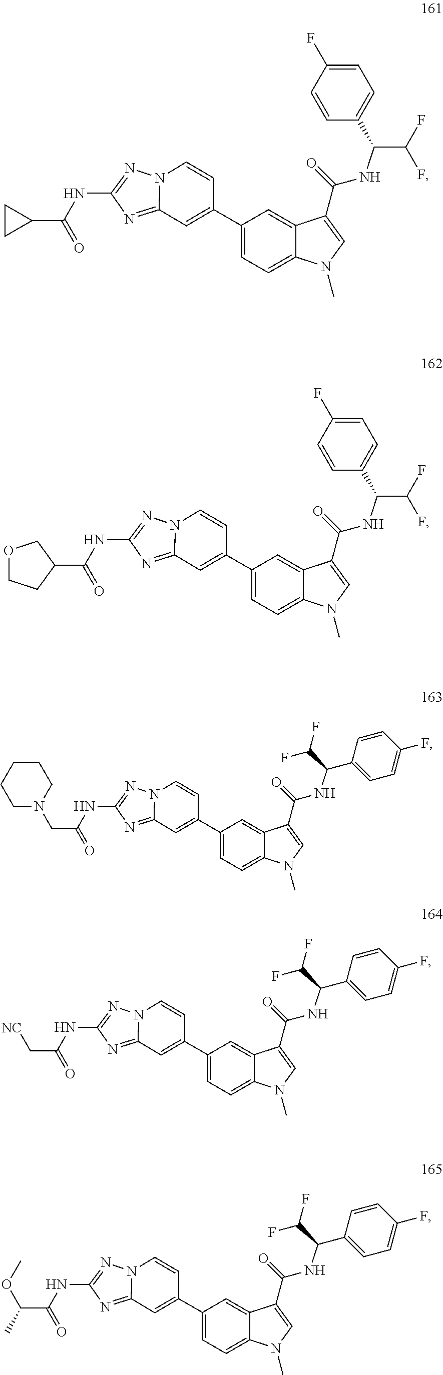 [SMILES: Cn1cc(C(=O)N[C@H](c2ccc(F)cc2)C(F)F)c2cc(-c3ccn4nc(NC(=O)C5CC5)nc4c3)ccc21 ; Cn1cc(C(=O)N[C@H](c2ccc(F)cc2)C(F)F)c2cc(-c3ccn4nc(NC(=O)C5CCOC5)nc4c3)ccc21 ; Cn1cc(C(=O)N[C@H](c2ccc(F)cc2)C(F)F)c2cc(-c3ccn4nc(NC(=O)CN5CCCCC5)nc4c3)ccc21 ; Cn1cc(C(=O)N[C@H](c2ccc(F)cc2)C(F)F)c2cc(-c3ccn4nc(NC(=O)CC#N)nc4c3)ccc21 ; CO[C@@H](C)C(=O)Nc1nc2cc(-c3ccc4c(c3)c(C(=O)N[C@H](c3ccc(F)cc3)C(F)F)cn4C)ccn2n1]

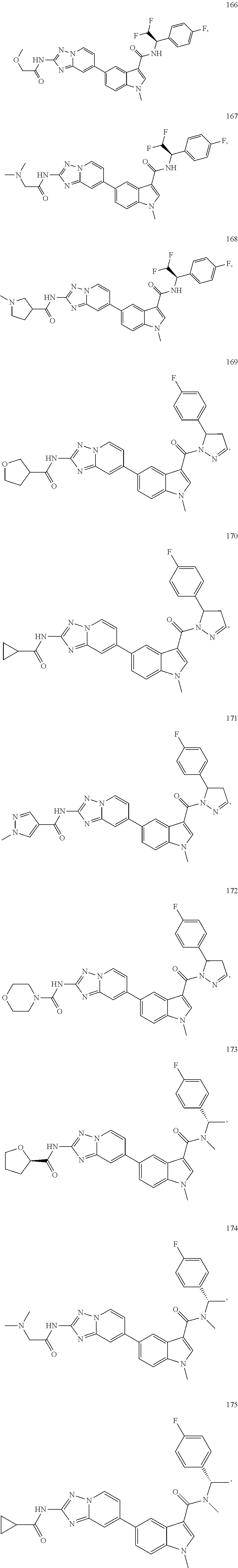

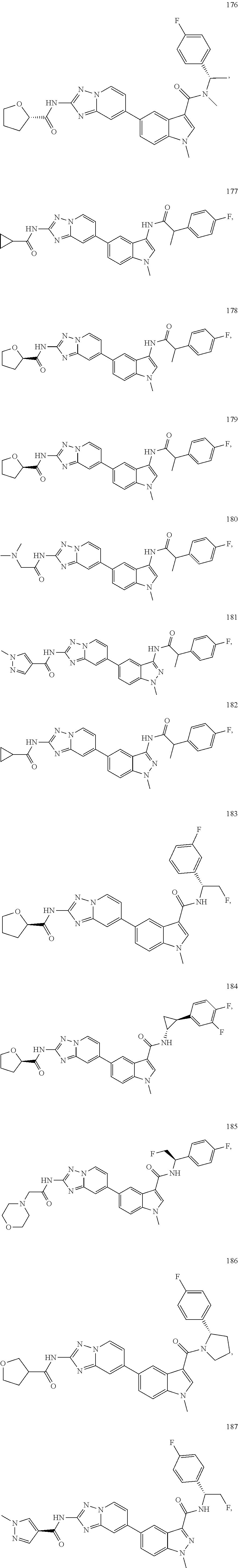

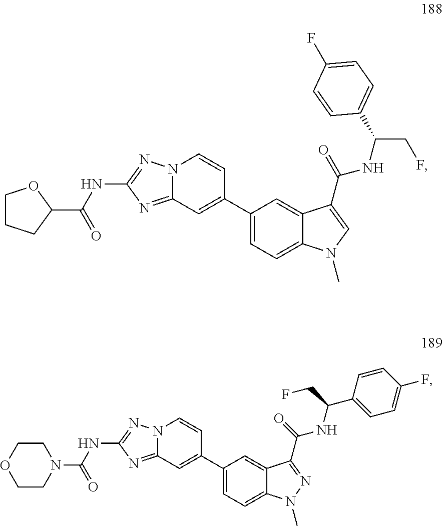 [SMILES: Cn1cc(C(=O)N[C@@H](CF)c2ccc(F)cc2)c2cc(-c3ccn4nc(NC(=O)C5CCCO5)nc4c3)ccc21 ; Cn1nc(C(=O)N[C@@H](CF)c2ccc(F)cc2)c2cc(-c3ccn4nc(NC(=O)N5CCOCC5)nc4c3)ccc21]

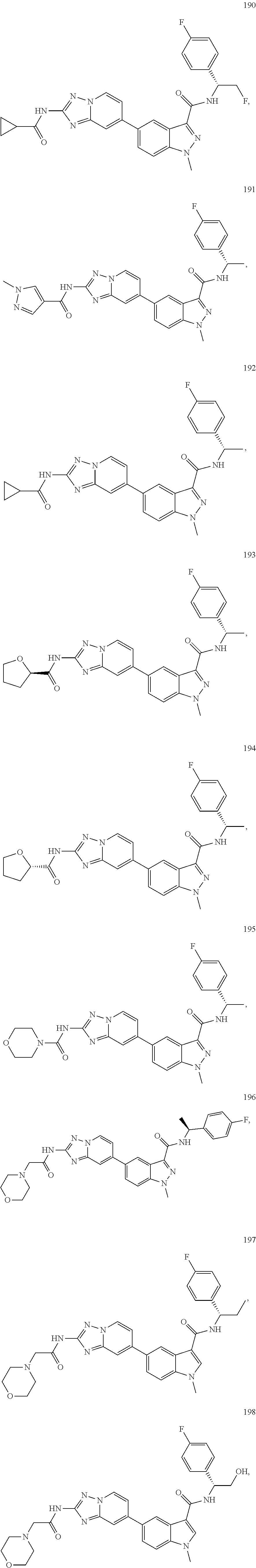

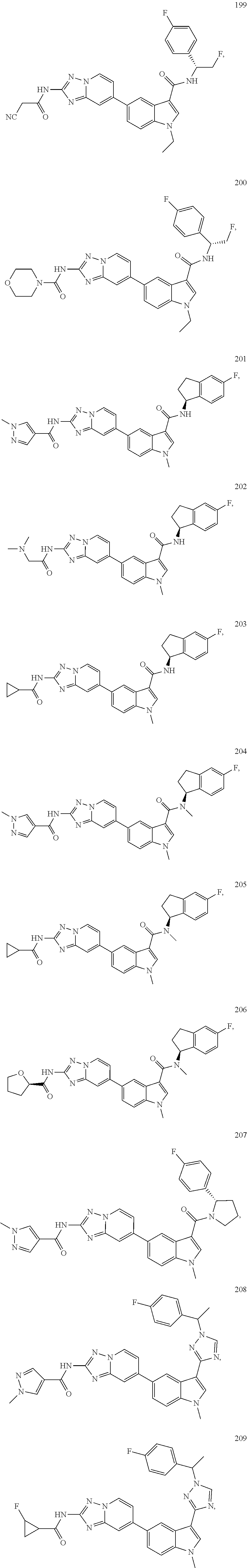

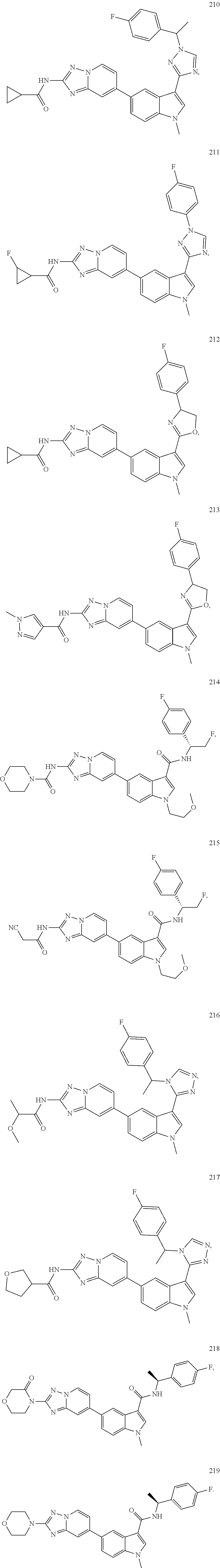

Another aspect of the present invention provides a pharmaceutical composition comprising the aforementioned compound, or a pharmaceutically acceptable salt, solvate or stereoisomer thereof.

Another aspect of the present invention provides the use of the aforementioned compound, or a pharmaceutically acceptable salt, solvate or stereoisomer thereof or a pharmaceutical composition thereof, in the manufacture of a medicament for preventing and/or treating inflammation, autoimmune diseases, neurodegenerative diseases and tumor-related diseases.

Preferably, it provides the use in the manufacture of a drug for treating tumor-related diseases.

Another aspect of the present invention provides the use of the aforementioned compound, or a pharmaceutically acceptable salt, solvate or stereoisomer thereof or a pharmaceutical composition thereof, in the manufacture of a RIPK1 and/or MLK inhibitor.

Preferably, the said MLK inhibitor is an MLK2/3 inhibitor.

A method for preventing and/or treating inflammation, autoimmune diseases, neurodegenerative diseases and tumor-related diseases, comprising the step of administering to a patient in need thereof the aforementioned compound, or a pharmaceutically acceptable salt, solvate or stereoisomer thereof or a pharmaceutical composition thereof.

Preferably, it comprises the step of administering a therapeutically effective dose of the aforementioned compound, or a pharmaceutically acceptable salt, solvate, stereoisomer or pharmaceutical composition thereof, to a patient in need thereof.

### DESCRIPTION OF EMBODIMENTS

### I. Definitions

In the present invention, scientific and technical terms used herein have the meanings commonly understood by those skilled in the art, unless otherwise specified. Furthermore, the relevant terms and laboratory operations used herein are widely used terms and routine steps in the corresponding field. Meanwhile, for a better understanding of the present invention, definitions and explanations of relevant terms are provided below.

As used herein and unless otherwise specified, the term “about” or “approximately” means within ±10% of a given value or range. Where an integer is required, the term refers to rounding to the nearest integer within ±10% of the given value or range. In the description herein, references to “some embodiments”, “some implementations” or “some schemes” describe a subset of all possible embodiments; however, it is understood that “some embodiments” may be the same subset or different subsets of all possible embodiments and may be combined with each other without conflict.

As used herein and unless otherwise specified, the terms “comprising”, “including”, “having”, “containing”, and grammatical equivalents thereof, should generally be understood as open-ended and non-restrictive, for example, not excluding other unrecited elements or steps.

As used herein, the term “pharmaceutically acceptable salt” refers to a salt of the compound in the present invention that is pharmaceutically acceptable and possesses the pharmacological activity of the parent compound. Such salt includes acid addition salt formed with inorganic acids or organic acids, such as nitric acid, phosphoric acid, carbonic acid, etc. as the said inorganic acids, or propionic acid, hexanoic acid, cyclopentylpropionic acid, glycolic acid, pyruvic acid, gluconic acid, stearic acid, muconic acid, etc. as the said organic acids; or salt formed when an acidic proton present in the parent compound is substituted with a metal ion, such as an alkali metal ion or an alkaline earth metal ion; or coordination compounds formed with an organic base, such as ethanolamine. The pharmaceutically acceptable salt in the present invention can be synthesized from a parent compound containing a basic or acidic moiety by conventional chemical methods. Generally, such salt is prepared by reacting the free acid or base forms of these compounds with a stoichiometric amount of the appropriate base or acid in water or in an organic solvent, or in a mixture thereof. Generally, non-aqueous media such as ether, ethyl acetate, ethanol, isopropanol, or acetonitrile are preferred. In addition to salt forms, the compound in the present invention also exists in the form of prodrug. The prodrug of the compound described herein readily undergo chemical changes under physiological conditions to convert into the compound in the present invention. In addition, the prodrug can be converted into the compound in the present invention using chemical or biochemical methods in an in vivo environment.

As used herein, the term “solvate” refers to a substance formed by the combination of a compound in the present invention with a pharmaceutically acceptable solvent. Pharmaceutically acceptable solvents include acetic acid, and the like. Solvates include stoichiometric solvates and non-stoichiometric solvates. Some compounds provided in the present invention may exist in non-solvate forms or solvate forms. In general, the solvate forms are equivalent to the non-solvate forms and are encompassed within the scope of the present invention.

As used herein, the term “stereoisomer” includes conformational isomers and configurational isomers, wherein the configurational isomers mainly include cis-trans isomers and optical isomers. The compound in the present invention may exist in the form of stereoisomer, and thus encompasses all possible stereoisomer forms, including but not limited to cis-trans isomers, tautomers, enantiomers, diastereoisomers, atropisomers. The compound in the present invention may also exist in the form of any combination or mixture of the aforementioned stereoisomers, such as meso compounds, racemates, equimolar mixture of atropisomers. For example, a single enantiomer, a single diastereomer or a mixture thereof, or a single atropisomer or a mixture thereof. When the compound in the present invention contains an olefinic double bond, unless otherwise specified, it includes cis-isomers and trans-isomers, as well as any combination thereof. The atropisomer in the present invention is a stereoisomer based on axial or planar chirality resulting from restricted intramolecular rotation. Furthermore, as a drug, the stereoisomer with excellent activity is preferred. The compound in the present invention possesses optical isomers derived from asymmetric carbons, etc. If necessary, a single isomer can be obtained by resolution using methods known in the art, such as crystallization or chiral chromatography.

As used herein, the term “heteroatom” is selected from nitrogen, oxygen, or sulfur. Wherein, the nitrogen may be optionally substituted; and the sulfur may also be optionally substituted, for example with oxo, i.e., forming S(O)m3 (wherein m3 is an integer from 0 to 2).

As used herein, the term “alkyl” refers to a straight or branched saturated aliphatic hydrocarbon group. The term “C₁₋₂₀ alkyl” refers to a straight or branched alkyl group having 1 to 20 carbon atoms. C₁₋₁₀ alkyl is preferred. C₁₋₆ alkyl (i.e., a straight or branched alkyl group having 1, 2, 3, 4, 5, or 6 carbon atoms) is more preferred. C₁₋₄ alkyl is more preferred. C₁₋₃ alkyl is more preferred. Specific examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, and various branched isomers thereof.

As used herein, the term “alkoxy” refers to a group having an —O-alkyl structure, wherein alkyl is as defined above. The term “C₁₋₆ alkoxy” refers to alkoxy having 1 to 6 carbon atoms. Preferably, it is C₁₋₄ alkoxy, and more preferably C₁₋₃ alkoxy. Specific examples include, but are not limited to, methoxy, ethoxy, n-propoxy, isopropoxy, n-butoxy, tert-butoxy, isobutoxy, n-pentyloxy, and the like.

As used herein, the term “cycloalkyl” refers to a saturated monocyclic or polycyclic hydrocarbon group, including, for example, monocyclic cycloalkyl, spirocycloalkyl, fused cycloalkyl, and bridged cycloalkyl. The ring carbon atoms of the cycloalkyl in the present invention may optionally be substituted with 1, 2 or 3 oxo groups to form a cyclic ketone structure. The term “3- to 20-membered cycloalkyl” or “C₃₋₂₀ cycloalkyl” refers to cycloalkyl having 3 to 20 ring carbon atoms, including monocyclic cycloalkyl, spirocycloalkyl, fused cycloalkyl and bridged cycloalkyl. C₃₋₁₂ cycloalkyl, C₅₋₂₀ spirocycloalkyl, C₅₋₂₀ fused cycloalkyl or C₅₋₂₀ bridged cycloalkyl is preferred. C₃₋₈ monocyclic cycloalkyl is more preferred.

As used herein, the term “spirocycloalkyl” refers to a saturated or partially unsaturated polycyclic hydrocarbon group in which one carbon atom (called a spiro atom) is shared between the rings. The term “spiroheterocycloalkyl” refers to spirocycloalkyl containing a C atom and at least one heteroatom selected from N, O and S as ring members. Non-limiting examples of spiroheterocycloalkyl include:

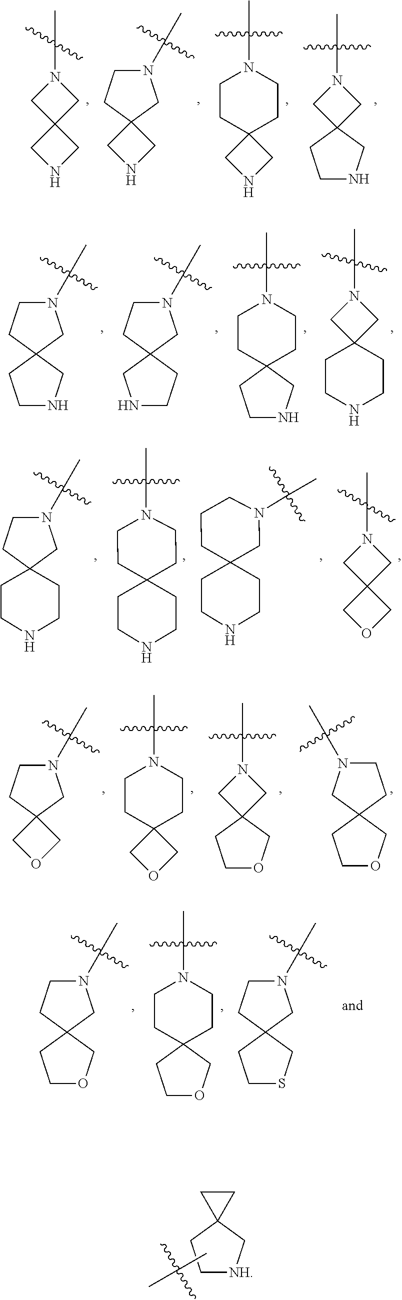

As used herein, the term “bridged cycloalkyl” refers to a saturated or partially unsaturated polycyclic hydrocarbon group in which any two rings share two carbon atoms that are not directly connected. The term “bridged heterocycloalkyl” refers to a bridged cycloalkyl containing C atoms and at least one heteroatom selected from N, O and S as ring members.

As used herein, the term “heterocycloalkyl” refers to a saturated ring substituent containing heteroatoms in the ring skeleton, wherein one or more (preferably 1 to 4, 1 to 3, or 1 to 2) ring atoms are heteroatoms selected from N, O, and S. The term “4- to 12-membered heterocycloalkyl” refers to a saturated ring substituent optionally containing 1, 2, 3, or 4 heteroatoms selected from N, O, and S as ring atoms. The term “4- to 12-membered nitrogen-containing heterocycloalkyl” refers to a 4- to 12-membered heterocycloalkyl in which one ring atom must be a nitrogen atom, and the remaining ring atoms are all carbon atoms or 0, 1, 2, or 3 of the remaining ring atoms are independently heteroatoms selected from N, O, and S. The said “4- to 12-membered nitrogen-containing heterocycloalkyl” is preferably attached to the remainder of the molecule via the mandatory nitrogen atom.

As used herein, the term “partially unsaturated heterocycle” refers to a cyclic group containing a C atom and at least one heteroatom selected from N, O, or S as ring members; wherein the bicyclic group is partially unsaturated and contains at least one C—C double bond, preferably one C—C double bond. As used herein, the term “partially unsaturated bicyclic ring” includes “partially unsaturated bicyclic carbocycle” and “partially unsaturated bicyclic heterocycle”, wherein the term “partially unsaturated bicyclic carbocycle” refers to a bicyclic group containing only C atoms as ring members, which is partially unsaturated and contains at least one C—C double bond; a maximally unsaturated carbocycle contains the maximum number of conjugated C—C double bonds allowed by the ring size, while a partially unsaturated bicyclic carbocycle contains fewer than the maximum number of C—C double bonds allowed by the ring size. The said “partially unsaturated bicyclic carbocycle” is preferably formed by the fusion of C₅₋₈ aryl with 5- or 6-membered monocyclic ring, wherein the said 5- or 6-membered monocyclic ring is selected from the following group:

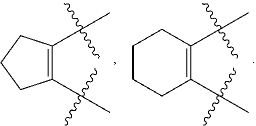 [SMILES: *C1=C(*)CCC1 ; *C1=C(*)CCCC1]

As used herein, the term “partially unsaturated bicyclic heterocycle” refers to a bicyclic group comprising a C atom and at least one heteroatom selected from N, O, and S as ring members, wherein the bicyclic group is partially unsaturated and contains at least one C—C double bond. A maximally unsaturated heterocycle comprises the maximum number of C—C double bonds and double bonds between C atoms and heteroatoms allowed by the ring size, whereas a partially unsaturated bicyclic heterocycle comprises fewer double bonds than the number allowed by the ring size.

As used herein, the term “aryl” refers to an all-carbon monocyclic, all-carbon non-fused polycyclic (rings connected via covalent bonds and not fused) or all-carbon fused polycyclic (i.e., rings sharing adjacent pairs of carbon atoms) group, wherein at least one ring in the group is aromatic, i.e., possessing a conjugated π-electron system. The term “C₆₋₁₄ aryl” refers to aryl having 6 to 14 ring atoms. C₆₋₁₀ aryl is preferred. In the present invention, C₆₋₁₄ aryl includes monocyclic aryl, non-fused polycyclic aryl and aromatic fused polycyclic aryl, wherein examples of monocyclic aryl include phenyl, and examples of non-fused polycyclic aryl include biphenyl, etc.

As used herein, the term “5- or 6-membered monocyclic heteroaryl” refers to monocyclic heteroaryl having 5 or 6 ring atoms, wherein 1, 2 or 3 ring atoms are heteroatoms selected from N, O and S(═O)m′ (where m′ is an integer from 0 to 2). Specific examples of monocyclic heteroaryl include, but are not limited to, thiophene, furan, thiazole, isothiazole, imidazole, oxazole, pyrrole, pyrazole, triazole, 1,2,3-triazole, 1,2,4-triazole, 1,2,5-triazole, 1,3,4-triazole, tetrazole, isoxazole, oxadiazole, 1,2,3-oxadiazole, 1,2,4-oxadiazole, 1,2,5-oxadiazole, 1,3,4-oxadiazole, thiadiazole, pyridine, pyridazine, pyrimidine, pyrazine.

“Optionally substituted with one or more . . . ” means that it may be substituted with one or more specified substituents (preferably 1, 2 or 3), or may be unsubstituted; regarding “more” in the phrase “one or more”, unless otherwise specified, the minimum value is 2, and the maximum value is the number of substitutable sites of the group to be substituted.

If substituents are described as being “each selected from” or “independently selected from” a group, each substituent is selected independently of the other. Therefore, each substituent may be the same as or different from the other substituent(s).

“Substitution” refers to the replacement of a hydrogen atom in a molecule with a different group.

“Member” refers to the number of skeletal atoms constituting a ring.

The term “bond” in the present invention refers to a direct bond, which can also be understood as “absent”.

“Azacycloalkyl” refers to cycloalkyl containing a nitrogen atom in the ring skeleton.

“Bridged cycloalkyl” refers to polycyclic cycloalkyl in which two rings share two adjacent carbon atoms.

“Spirocycloalkyl” refers to cycloalkyl in which two carbon rings in the molecule share one carbon atom, and the shared carbon atom is referred to as a “spiro carbon atom”.

Unless otherwise specified, a ring formed by “connecting to form a ring” in the present invention includes unsubstituted rings and rings substituted with substituents.

“Alkoxy” refers to —O-alkyl.

“Halogen” refers to fluorine, chlorine, bromine, or iodine.

“(O)” or “oxo” refers to ═O.

“ [symbol] ” represents the connecting position of a chemical bond.

Although the term “optionally substituted” may not appear, if a substitution occurs at any position on a ring (such as an aliphatic ring or an aromatic ring), it should be understood that the position and number of the substituents are arbitrary, provided that they are chemically feasible.

### II. Examples

In order to make the purpose, technical solutions, and advantages of the present invention clearer, the present invention is described in further detail below. The described embodiments should not be construed as limiting the present invention. All other embodiments obtained by those ordinarily skilled persons in the art without creative efforts fall within the protection scope of the present invention.

Before describing the embodiments of the present invention in further detail, terms and terminology involved in the embodiments of the present invention are explained. The terms and terminology involved in the embodiments of the present invention are applicable to the following explanations.

The raw materials and equipment used in the specific embodiments of the present disclosure are all known products and are obtained by purchasing commercially available products.

### Preparation Examples

### Preparation of Intermediate INT-1

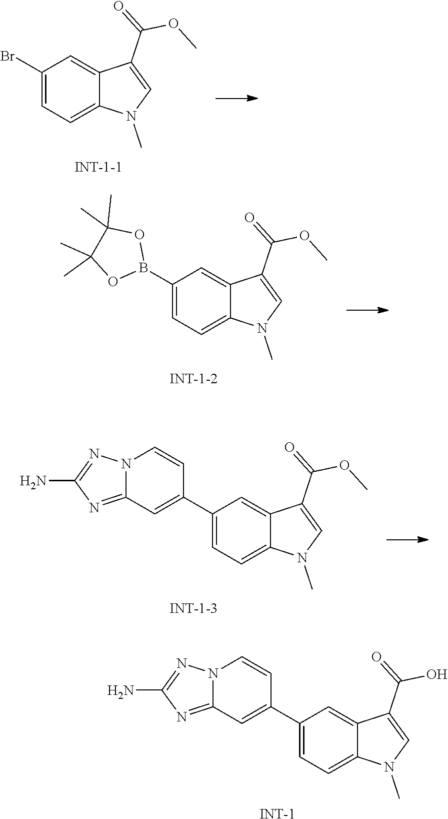

### Intermediate INT-1-2: Preparation of methyl 1-methyl-5-(4,4,5,5-tetramethyl-1,3,2-dioxaborolan-2-yl)-1H-indol-3-carboxylate

Under an argon atmosphere, Intermediate INT-1-1 (1335 mg, 5.0 mmol) and bis(pinacolato) diboron (1651 mg, 6.5 mmol) were dissolved in 1,4-dioxane (20 mL), and potassium acetate (4.9 g, 10.0 mmol) and PdCl₂(dppf)₂ (160 mg) were added; three cycles of argon purging was performed, and the mixture was heated to 100° C. and stirred for 10 h. The reaction mixture was concentrated under reduced pressure and separated and purified by silica gel column (petroleum ether/ethyl acetate=5/1) to obtain Intermediate INT-1-2 with a yield of 80%. MS (ESI, positive ion) m/z: 316.3 [M+H]⁺.

### Intermediate INT-1-3: Preparation of methyl 5-(2-amino-[1,2,4]triazolo[1,5-a]pyridin-7-yl)-1-methyl-1H-indol-3-carboxylate

Under an argon atmosphere, Intermediate INT-1-2 (1260 mg, 4.0 mmol) and 7-bromo-[1,2,4]triazolo[1,5-a]pyridin-2-amine (848 mg, 4.0 mmol) were dissolved in 1,4-dioxane (20 mL), potassium phosphate (1.7 g, 8.0 mmol), PdCl₂(dppf)₂ (120 mg), and water (2.0 mL) were added; three cycles of argon purging was performed, the mixture was heated to 95° C. and stirred for 8 h. The reaction mixture was concentrated under reduced pressure and separated and purified by silica gel column (dichloromethane/methanol=20/1) to obtain Intermediate INT-1-3 with a yield of 70%. MS (ESI, positive ion) m/z: 322.2 [M+H]⁺.

### Intermediate INT-1: Preparation of 5-(2-amino-[1,2,4]triazolo[1,5-a]pyridin-7-yl)-1-methyl-1H-indol-3-carboxylic acid

Intermediate INT-1-3 (963 mg, 3.0 mmol) was dissolved in ethanol (3 mL), tetrahydrofuran (3 mL), and water (15 mL); sodium hydroxide (0.96 g, 24 mmol) was added; the mixture was heated to 75° C. and stirred for 5 h. The pH was adjusted to slightly acidic with dilute hydrochloric acid (1 M). The mixture was filtered and dried to obtain Intermediate INT-1 with a yield of 92%. MS (ESI, positive ion) m/z: 308.3 [M+H]⁺.

### Preparation of Intermediate INT-2

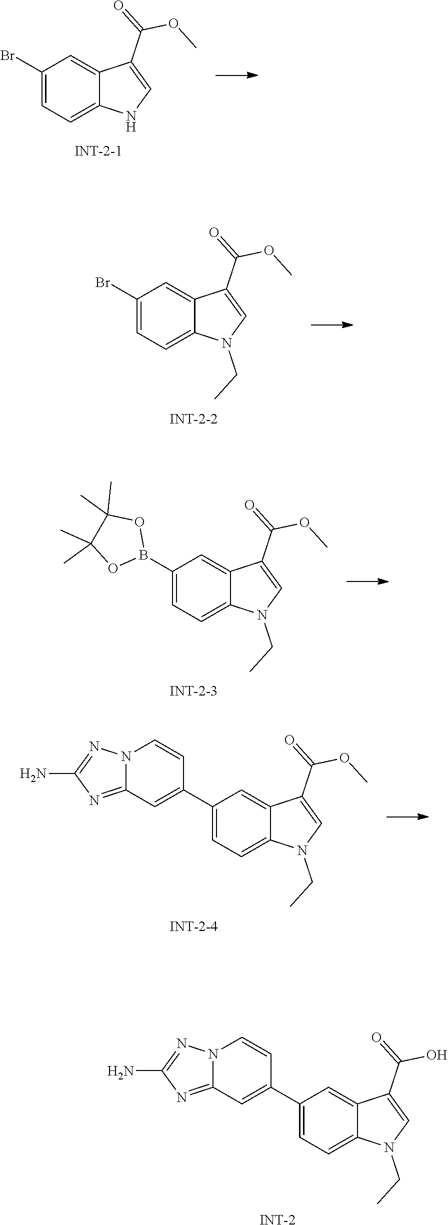

### Intermediate INT-2-2: Preparation of methyl 5-bromo-1-ethyl-1H-indol-3-carboxylate

Intermediate INT-2-1 (2.0 g, 8.0 mmol) was dissolved in N,N-dimethylformamide (20 mL), ethyl iodide (2.5 g, 16.0 mmol) and sodium hydride (640 mg, 16 mmol, 60%) were added in an ice bath; the mixture was stirred at room temperature for 2 h. Water (20 mL) was added in an ice bath, and the mixture was extracted with ethyl acetate twice. The pooled organic phases were dried over anhydrous sodium sulfate, filtered, and concentrated under reduced pressure. The residue was separated by silica gel column chromatography (petroleum ether/ethyl acetate=10/1) to obtain Intermediate INT-2-2 with a yield of 83%. MS (ESI, positive ion) m/z: 282.2 [M+H]⁺.

### Intermediate INT-2-3: Preparation of 1-ethyl-5-(4,4,5,5-tetramethyl-1,3,2-dioxaborolan-2-yl)-1H-indol-3-carboxylic acid methyl ester

Under an argon atmosphere, Intermediate INT-2-2 (1.4 g, 5.0 mmol) and bis(pinacolato) diboron (1651 mg, 6.5 mmol) were dissolved in 1,4-dioxane (20 mL), and potassium acetate (4.9 g, 10.0 mmol) and PdCl₂(dppf)₂ (160 mg) were added; three cycles of argon purging was performed, and the mixture was heated to 100° C. and stirred for 10 h. The reaction mixture was concentrated under reduced pressure and separated and purified by silica gel column (petroleum ether/ethyl acetate=5/1) to obtain Intermediate INT-2-3 with a yield of 80%. MS (ESI, positive ion) m/z: 330.4 [M+H]⁺.

### Intermediate INT-2-4: Preparation of 5-(2-amino-[1,2,4]triazolo[1,5-a]pyridin-7-yl)-1-ethyl-1H-indol-3-carboxylic acid methyl ester

Under an argon atmosphere, Intermediate INT-2-3 (1.3 g, 4.0 mmol) and 7-bromo-[1,2,4]triazolo[1,5-a]pyridin-2-amine (848 mg, 4.0 mmol) were dissolved in 1,4-dioxane (20 mL), potassium phosphate (1.7 g, 8.0 mmol), PdCl₂(dppf)₂ (120 mg), and water (2.0 mL) were added; three cycles of argon purging was performed, the mixture was heated to 95° C. and stirred for 8 h. The reaction mixture was concentrated under reduced pressure and separated and purified by silica gel column (dichloromethane/methanol=20/1) to obtain Intermediate INT-2-4 with a yield of 70%. MS (ESI, positive ion) m/z: 322.2 [M+H]⁺.

### Intermediate INT-2: Preparation of 5-(2-amino-[1,2,4]triazolo[1,5-a]pyridin-7-yl)-1-ethyl-1H-indol-3-carboxylic acid

Intermediate INT-2-4 (1.0 g, 3.0 mmol) was dissolved in ethanol (3 mL), tetrahydrofuran (3 mL), and water (15 mL); sodium hydroxide (0.96 g, 24 mmol) was added; the mixture was heated to 75° C. and stirred for 5 h. The pH was adjusted to slightly acidic with dilute hydrochloric acid (1 M). The mixture was filtered and dried to obtain Intermediate INT-2 with a yield of 92%. MS (ESI, positive ion) m/z: 322.2 [M+H]⁺.

### Preparation of Intermediate INT-3

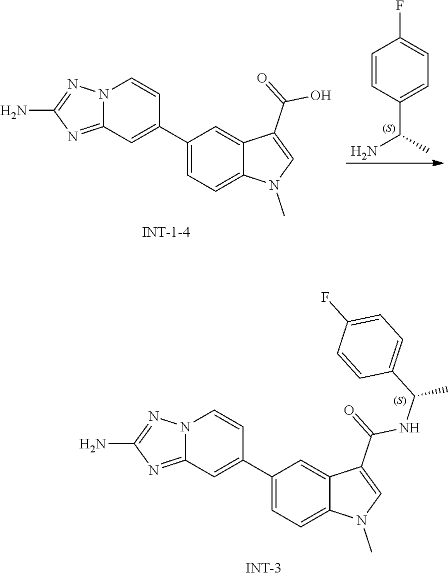

### Intermediate INT-3: Preparation of (S)-5-(2-amino-[1,2,4]triazolo[1,5-a]pyridin-7-yl)-N-[1-(4-fluorophenyl)ethyl]-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(4-fluorophenyl)ethan-1-amine (167 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-3 with a yield of 70%. MS (ESI, positive ion) m/z: 429.3 [M+H]⁺.

### Preparation of Intermediate INT-4

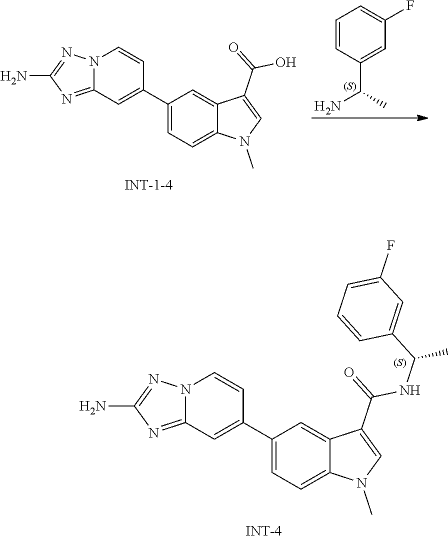

### Intermediate INT-4: Preparation of (S)-5-(2-amino-[1,2,4]triazolo[1,5-a]pyridin-7-yl)-N-[1-(3-fluorophenyl)ethyl]-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(3-fluorophenyl)ethan-1-amine (167 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-4 with a yield of 71%. MS (ESI, positive ion) m/z: 429.3 [M+H]⁺.

### Preparation of Intermediate INT-5

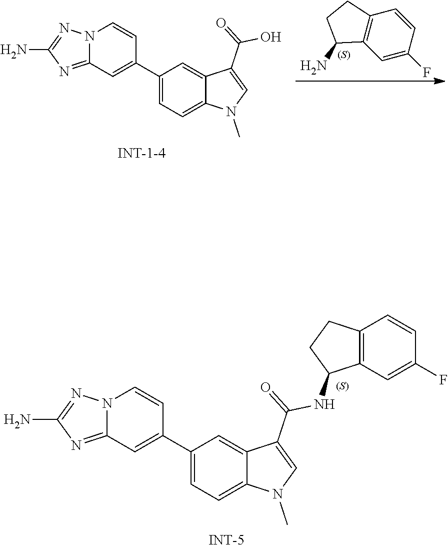

### Intermediate INT-5: Preparation of (S)-5-(2-amino-[1,2,4]triazolo[1,5-a]pyridin-7-yl)-N-(6-fluoro-2,3-dihydro-1H-inden-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-6-fluoro-2,3-dihydro-1H-inden-1-amine (181 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-5 with a yield of 73%. MS (ESI, positive ion) m/z: 441.2 [M+H]⁺.

### Preparation of Intermediate INT-6

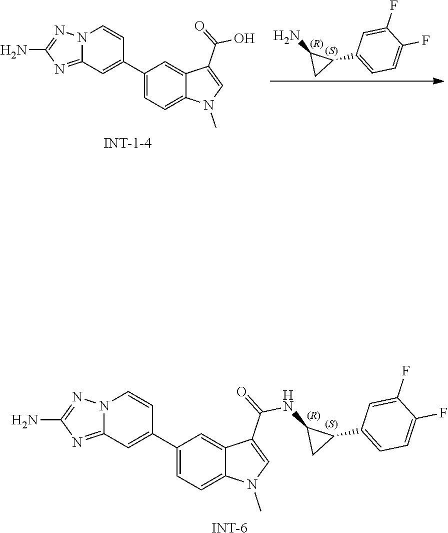

### Intermediate INT-6: Preparation of 5-(2-amino-[1,2,4]triazolo[1,5-a]pyridin-7-yl)-N-[(1R,2S)-2-(3,4-difluorophenyl)cyclopropyl]-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (1R,2S)-2-(3,4-difluorophenyl)cyclopropan-1-amine (202 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-6 with a yield of 68%. MS (ESI, positive ion) m/z: 459.3 [M+H]⁺.

### Preparation of Intermediate INT-7

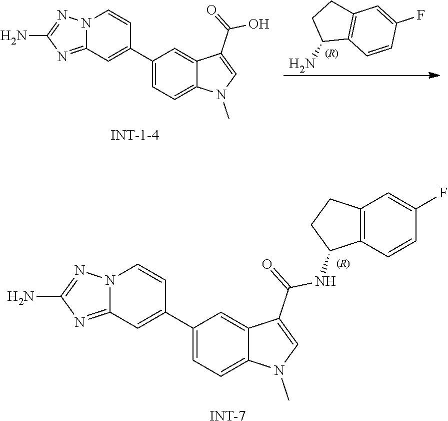

### Intermediate INT-7: Preparation of (R)-5-(2-amino-[1,2,4]triazolo[1,5-a]pyridin-7-yl)-N-(5-fluoro-2,3-dihydro-1H-inden-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-5-fluoro-2,3-dihydro-1H-inden-1-amine (181 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-7 with a yield of 68%. MS (ESI, positive ion) m/z: 443.3 [M+H]⁺.

### Preparation of Intermediate INT-8

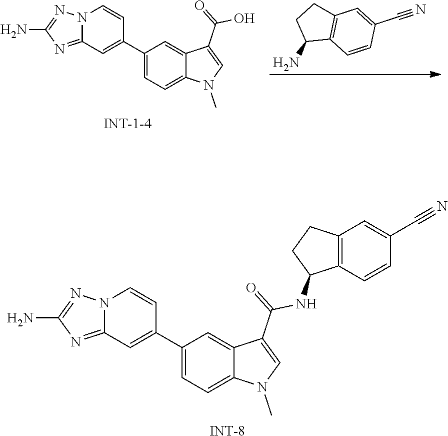 [SMILES: Cn1cc(C(=O)O)c2cc(-c3ccn4nc(N)nc4c3)ccc21 ; N#Cc1ccc2c(c1)CC[C@@H]2N ; "INT-1-4" ; Cn1cc(C(=O)N[C@H]2CCc3cc(C#N)ccc32)c2cc(-c3ccn4nc(N)nc4c3)ccc21 ; "INT-8"]

### Intermediate INT-8: Preparation of (S)-5-(2-amino-[1,2,4]triazolo[1,5-a]pyridin-7-yl)-N-(5-cyano-2,3-dihydro-1H-inden-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-amino-2,3-dihydro-1H-inden-5-carbonitrile (181 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-8 with a yield of 66%. MS (ESI, positive ion) m/z: 448.3 [M+H]⁺.

### Preparation of Intermediate INT-9

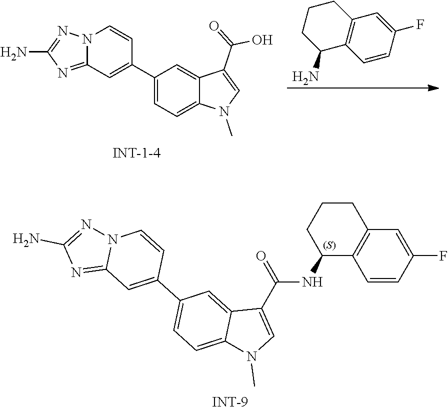

### Intermediate INT-9: Preparation of (S)-5-(2-amino-[1,2,4]triazolo[1,5-a]pyridin-7-yl)-N-(6-fluoro-1,2,3,4-tetrahydronaphthalen-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-6-fluoro-1,2,3,4-tetrahydronaphthalen-1-amine (198 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-9 with a yield of 69%. MS (ESI, positive ion) m/z: 455.3 [M+H]⁺.

### Preparation of Intermediate INT-10

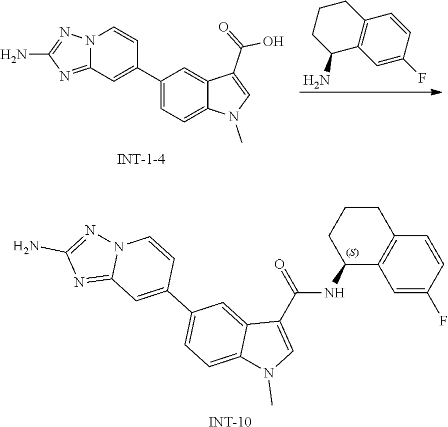

### Intermediate INT-10: Preparation of (S)-5-(2-amino-[1,2,4]triazolo[1,5-a]pyridin-7-yl)-N-(7-fluoro-1,2,3,4-tetrahydronaphthalen-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-7-fluoro-1,2,3,4-tetrahydronaphthalen-1-amine (198 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-10 with a yield of 68%. MS (ESI, positive ion) m/z: 455.3 [M+H]⁺.

### Preparation of Intermediate INT-11

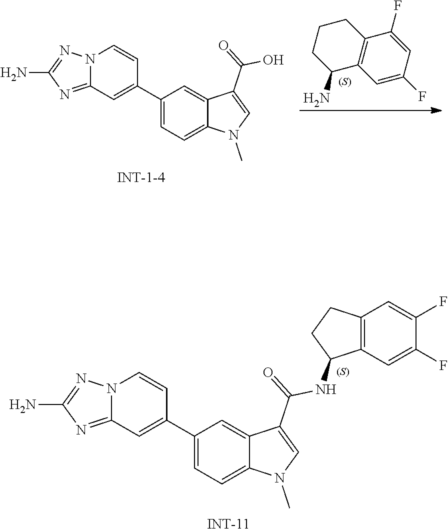

### Intermediate INT-11: Preparation of (S)-5-(2-amino-[1,2,4]triazolo[1,5-a]pyridin-7-yl)-N-(5,6-difluoro-2,3-dihydro-1H-inden-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-5,6-difluoro-2,3-dihydro-1H-inden-1-amine (203 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-11 with a yield of 65%. MS (ESI, positive ion) m/z: 459.3 [M+H]⁺.

### Preparation of Intermediate INT-12

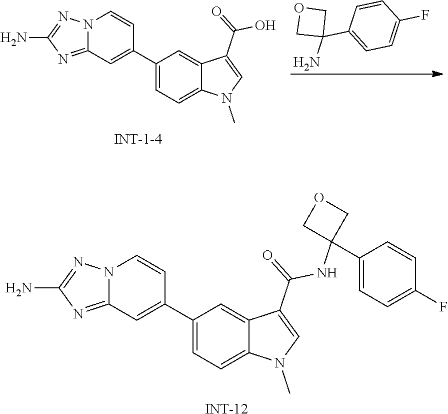

### Intermediate INT-12: Preparation of 5-(2-amino-[1,2,4]triazolo[1,5-a]pyridin-7-yl)-N-(3-(4-fluorophenyl)oxetan-3-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. 3-(4-fluorophenyl)oxetan-3-amine (200 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-12 with a yield of 65%. MS (ESI, positive ion) m/z: 457.3 [M+H]⁺.

### Preparation of Intermediate INT-13

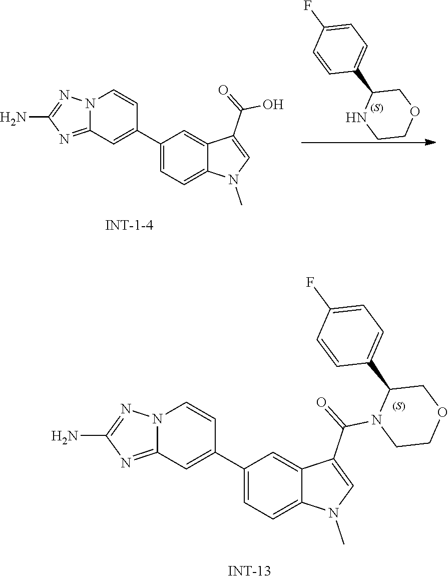

### Intermediate INT-13: Preparation of (S)-(5-(2-amino-[1,2,4]triazolo[1,5-a]pyridin-7-yl)-1-methyl-1H-indol-3-yl)(3-(4-fluorophenyl)morpholino)methanone

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-3-(4-fluorophenyl)morpholine (217 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-13 with a yield of 71%. MS (ESI, positive ion) m/z: 471.3 [M+H]⁺.

### Preparation of Intermediate INT-14

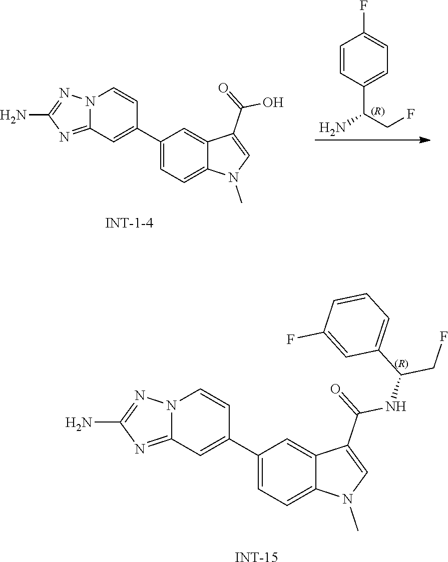

### Intermediate INT-14: Preparation of (R)-5-(2-amino-[1,2,4]triazolo[1,5-a]pyridin-7-yl)-N-(2-fluoro-1-(3-fluorophenyl)ethyl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-2-fluoro-1-(3-fluorophenyl)ethan-1-amine (188 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-14 with a yield of 72%. MS (ESI, positive ion) m/z: 447.3 [M+H]⁺.

### Preparation of Intermediate INT-15

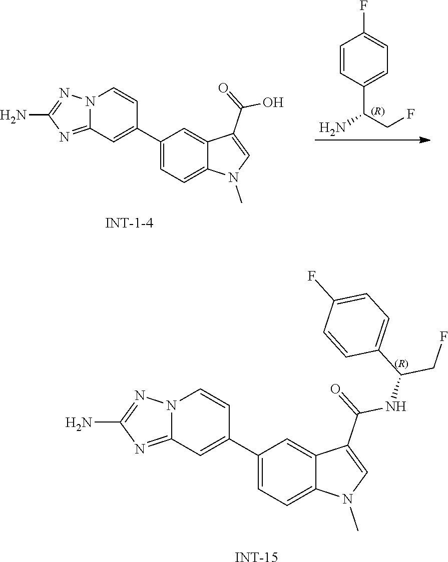

### Intermediate INT-15: Preparation of (R)-5-(2-amino-[1,2,4]triazolo[1,5-a]pyridin-7-yl)-N-(2-fluoro-1-(4-fluorophenyl)ethyl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-2-fluoro-1-(4-fluorophenyl)ethan-1-amine (188 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-15 with a yield of 72%. MS (ESI, positive ion) m/z: 447.3 [M+H]⁺.

### Preparation of Intermediate INT-16

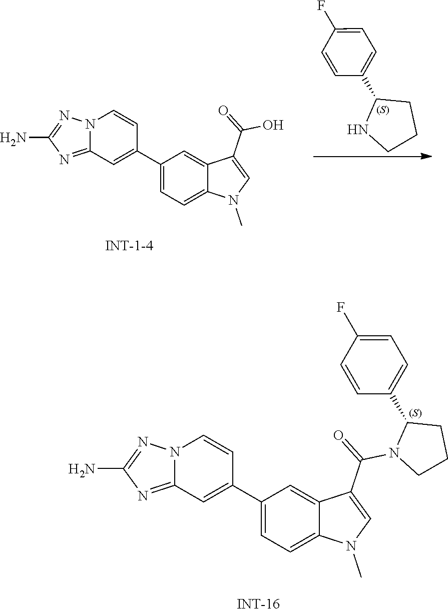

### Intermediate INT-16: Preparation of (S)-(5-(2-amino-[1,2,4]triazolo[1,5-a]pyridin-7-yl)-1-methyl-1H-indol-3-yl)(2-(4-fluorophenyl)pyrrolidin-1-yl)methanone

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-2-(4-fluorophenyl)pyrrolidine (198 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-16 with a yield of 73%. MS (ESI, positive ion) m/z: 455.3 [M+H]⁺.

### Preparation of Intermediate INT-17

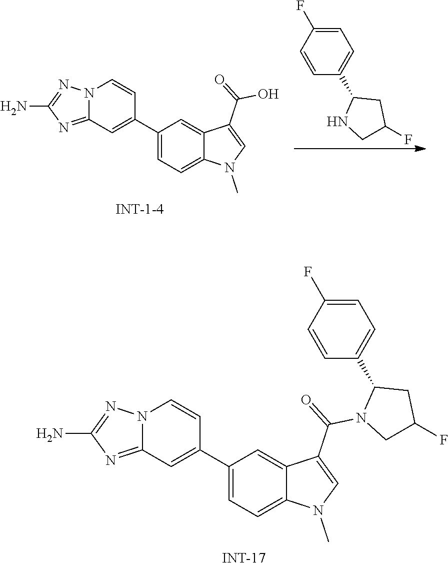 [SMILES: Cn1cc(C(=O)O)c2cc(-c3ccn4nc(N)nc4c3)ccc21 ; Fc1ccc([C@@H]2CC(F)CN2)cc1 ; "INT-1-4" ; Cn1cc(C(=O)N2CC(F)C[C@H]2c2ccc(F)cc2)c2cc(-c3ccn4nc(N)nc4c3)ccc21 ; "INT-17"]

### Intermediate INT-17: Preparation of (5-(2-amino-[1,2,4]triazolo[1,5-a]pyridin-7-yl)-1-methyl-1H-indol-3-yl)((2S)-4-fluoro-2-(4-fluorophenyl)pyrrolidin-1-yl)methanone

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (2S)-4-fluoro-2-(4-fluorophenyl)pyrrolidine (220 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-17 with a yield of 70%. MS (ESI, positive ion) m/z: 473.3 [M+H]⁺.

### Preparation of Intermediate INT-18

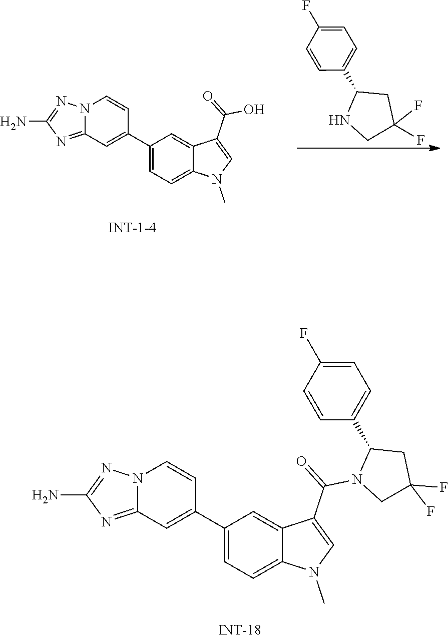 [SMILES: Cn1cc(C(=O)O)c2cc(-c3ccn4nc(N)nc4c3)ccc21 ; Fc1ccc([C@@H]2CC(F)(F)CN2)cc1 ; Cn1cc(C(=O)N2CC(F)(F)C[C@H]2c2ccc(F)cc2)c2cc(-c3ccn4nc(N)nc4c3)ccc21 ; "INT-18"]

### Intermediate INT-18: Preparation of (S)-(5-(2-amino-[1,2,4]triazolo[1,5-a]pyridin-7-yl)-1-methyl-1H-indol-3-yl)(4,4-difluoro-2-(4-fluorophenyl)pyrrolidin-1-yl)methanone

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-4,4-difluoro-2-(4-fluorophenyl)pyrrolidine (241 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-18 with a yield of 70%. MS (ESI, positive ion) m/z: 491.3 [M+H]⁺.

### Preparation of Intermediate INT-19

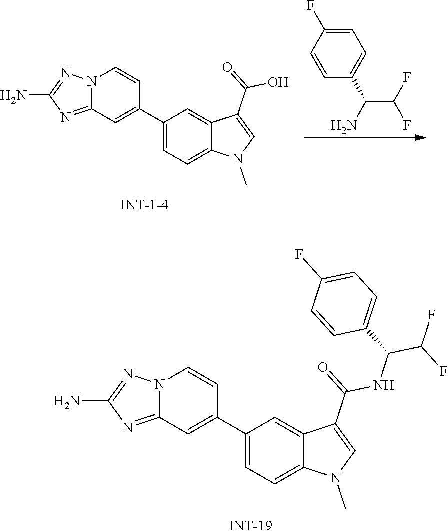 [SMILES: Cn1cc(C(=O)O)c2cc(-c3ccn4nc(N)nc4c3)ccc21 ; N[C@H](c1ccc(F)cc1)C(F)F ; Cn1cc(C(=O)N[C@H](c2ccc(F)cc2)C(F)F)c2cc(-c3ccn4nc(N)nc4c3)ccc21 ; "INT-19"]

### Intermediate INT-19: Preparation of (R)-5-(2-amino-[1,2,4]triazolo[1,5-a]pyridin-7-yl)-N-(2,2-difluoro-1-(4-fluorophenyl)ethyl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-2,2-difluoro-1-(4-fluorophenyl)ethan-1-amine (210 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-19 with a yield of 70%. MS (ESI, positive ion) m/z: 464.3 [M+H]⁺.

### Preparation of Intermediate INT-20

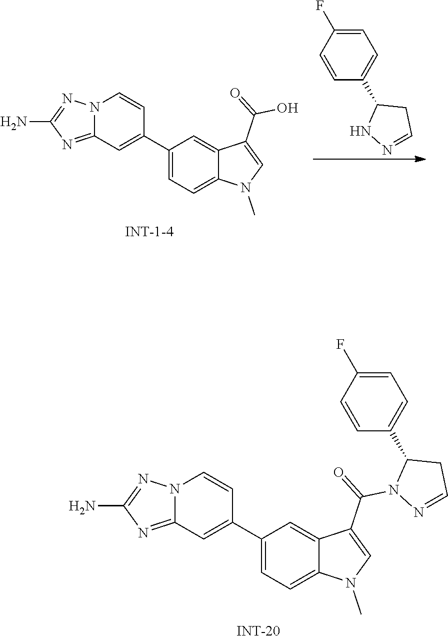 [SMILES: Cn1cc(C(=O)O)c2cc(-c3ccn4nc(N)nc4c3)ccc21 ; Fc1ccc([C@@H]2CC=NN2)cc1 ; "INT-1-4" ; Cn1cc(C(=O)N2N=CC[C@H]2c2ccc(F)cc2)c2cc(-c3ccn4nc(N)nc4c3)ccc21 ; "INT-20"]

### Intermediate INT-20: Preparation of (S)-(5-(2-amino-[1,2,4]triazolo[1,5-a]pyridin-7-yl)-1-methyl-1H-indol-3-yl)(5-(4-fluorophenyl)-4,5-dihydro-1H-pyrazol-1-yl)methanone

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-5-(4-fluorophenyl)-4,5-dihydro-1H-pyrazole (197 mg, 1.2 mmol) was added and stirred for reaction at room temperature for 2 h. The reaction mixture was added dropwise to water and stirred for 0.5 h. The mixture was filtered and dried to obtain Intermediate INT-20 with a yield of 70%. MS (ESI, positive ion) m/z: 464.3 [M+H]⁺.

### Preparation of Intermediate INT-21

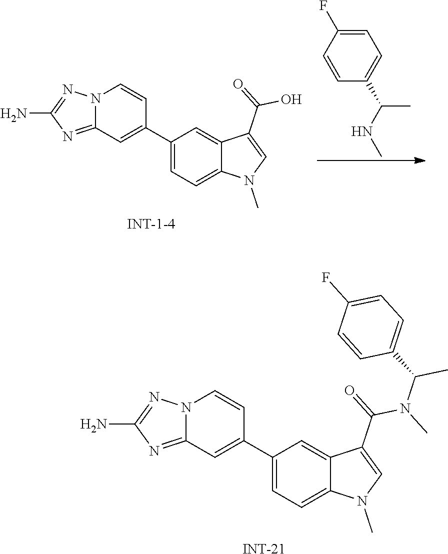 [SMILES: Cn1cc(C(=O)O)c2cc(-c3ccn4nc(N)nc4c3)ccc21 ; CN[C@@H](C)c1ccc(F)cc1 ; "INT-1-4" ; C[C@@H](c1ccc(F)cc1)N(C)C(=O)c1cn(C)c2ccc(-c3ccn4nc(N)nc4c3)cc12 ; "INT-21"]

### Intermediate INT-21: Preparation of (S)-5-(2-amino-[1,2,4]triazolo[1,5-a]pyridin-7-yl)-N-(1-(4-fluorophenyl)ethyl)-N,1-dimethyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(4-fluorophenyl)-N-methylethan-1-amine (184 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-21 with a yield of 70%. MS (ESI, positive ion) m/z: 443.3 [M+H]⁺.

### Preparation of Intermediate INT-22

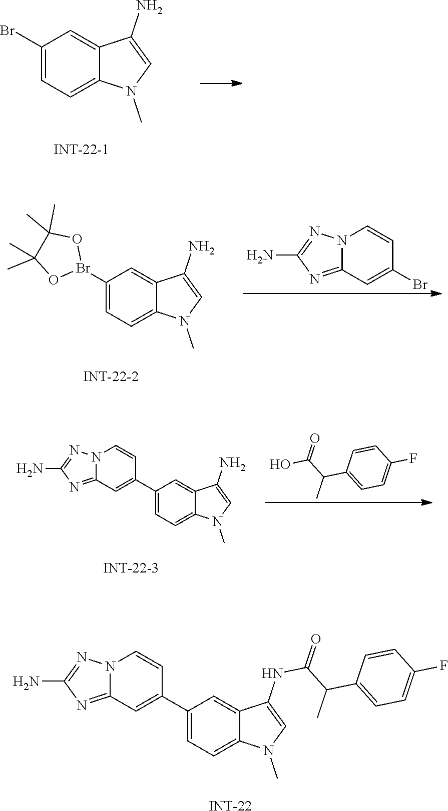

### Intermediate INT-22-2: Preparation of 1-methyl-5-(4,4,5,5-tetramethyl-1,3,2-dioxaborolan-2-yl)-1H-indol-3-amine

Under an argon atmosphere, Intermediate INT-22-1 (1.12 g, 5.0 mmol) and bis(pinacolato) diboron (1651 mg, 6.5 mmol) were dissolved in 1,4-dioxane (20 mL), and potassium acetate (4.9 g, 10.0 mmol) and PdCl₂(dppf)₂ (160 mg) were added; three cycles of argon purging was performed, and the mixture was heated to 100° C. and stirred for 10 h. The reaction mixture was concentrated under reduced pressure and separated and purified by silica gel column (petroleum ether/ethyl acetate=1/1) to obtain Intermediate INT-22-2 with a yield of 70%. MS (ESI, positive ion) m/z: 273.3 [M+H]⁺.

### Intermediate INT-22-3: Preparation of 7-(3-amino-1-methyl-H-indol-5-yl)-[1,2,4]triazolo[1,5-a]pyridin-2-amine

Under an argon atmosphere, Intermediate INT-22-2 (1.09 g, 4.0 mmol) and 7-bromo-[1,2,4]triazolo[1,5-a]pyridin-2-amine (848 mg, 4.0 mmol) were dissolved in 1,4-dioxane (20 mL), potassium phosphate (1.7 g, 8.0 mmol), PdCl₂(dppf)₂ (120 mg), and water (2.0 mL) were added; three cycles of argon purging was performed, the mixture was heated to 95° C. and stirred for 8 h. The reaction mixture was concentrated under reduced pressure and separated and purified by silica gel column (dichloromethane/methanol=20/1) to obtain Intermediate INT-22-3 with a yield of 61%. MS (ESI, positive ion) m/z: 279.2 [M+H]⁺.

### Intermediate INT-22: Preparation of N-(5-(2-amino-[1,2,4]triazolo[1,5-a]pyridin-7-yl)-1-methyl-1H-indol-3-yl)-2-(4-fluorophenyl)propanamide

Intermediate INT-22-3 (278 mg, 1.0 mmol) and 2-(4-fluorophenyl)propionic acid (168 mg, 1.0 mmol) were dissolved in dimethyl sulfoxide (10 mL); N,N-diisopropylethylamine (258 mg, 2 mmol) and HATU (570 mg, 1.5 mmol) were added, and the mixture was stirred at room temperature for 2 h. The reaction mixture was added dropwise into water and extracted with ethyl acetate. The organic phase was dried over anhydrous sodium sulfate, filtered, and concentrated under reduced pressure. The residue was separated and purified by silica gel column (dichloromethane/methanol=20/1) to obtain Intermediate INT-22 with a yield of 73%. MS (ESI, positive ion) m/z: 429.3. [M+H]⁺.

### Preparation of Intermediate INT-23

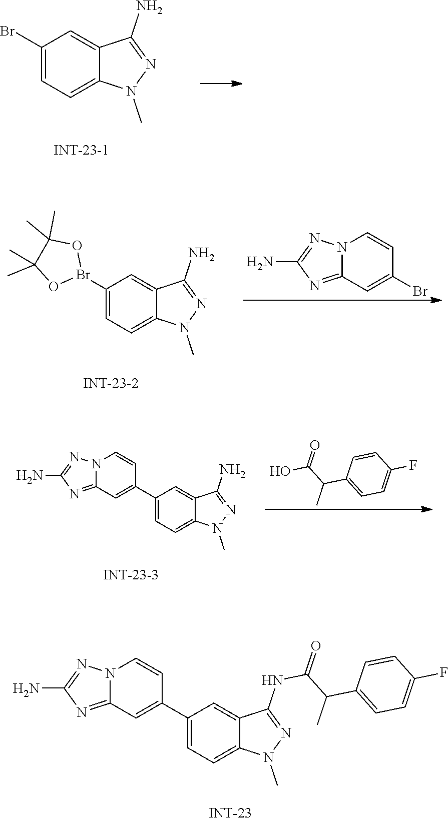

### Intermediate INT-23-2: Preparation of 1-methyl-5-(4,4,5,5-tetramethyl-1,3,2-dioxaborolan-2-yl)-1H-indazol-3-amine

Under an argon atmosphere, Intermediate INT-23-1 (1.12 g, 5.0 mmol) and bis(pinacolato) diboron (1651 mg, 6.5 mmol) were dissolved in 1,4-dioxane (20 mL), and potassium acetate (4.9 g, 10.0 mmol) and PdCl2(dppf)₂ (160 mg) were added; three cycles of argon purging was performed, and the mixture was heated to 100° C. and stirred for 10 h. The reaction mixture was concentrated under reduced pressure and separated and purified by silica gel column (petroleum ether/ethyl acetate=1/1) to obtain Intermediate INT-23-2 with a yield of 67%. MS (ESI, positive ion) m/z: 274.3 [M+H]⁺.

### Intermediate INT-23-3: Preparation of 7-(3-amino-1-methyl-1H-indazol-5-yl)-[1,2,4]triazolo[1,5-a]pyridin-2-amine

Under an argon atmosphere, Intermediate INT-23-2 (1.09 g, 4.0 mmol) and 7-bromo-[1,2,4]triazolo[1,5-a]pyridin-2-amine (848 mg, 4.0 mmol) were dissolved in 1,4-dioxane (20 mL), potassium phosphate (1.7 g, 8.0 mmol), PdCl₂(dppf)₂ (120 mg), and water (2.0 mL) were added; three cycles of argon purging was performed, the mixture was heated to 95° C. and stirred for 8 h. The reaction mixture was concentrated under reduced pressure and separated and purified by silica gel column (dichloromethane/methanol=20/1) to obtain Intermediate INT-23-3 with a yield of 61%. MS (ESI, positive ion) m/z: 280.2 [M+H]⁺.

### Intermediate INT-23: Preparation of N-(5-(2-amino-[1,2,4]triazolo[1,5-a]pyridin-7-yl)-1-methyl-1H-indazol-3-yl)-2-(4-fluorophenyl)propanamide

Intermediate INT-23-3 (278 mg, 1.0 mmol) and 2-(4-fluorophenyl)propionic acid (168 mg, 1.0 mmol) were dissolved in dimethyl sulfoxide (10 mL); N,N-diisopropylethylamine (258 mg, 2 mmol) and HATU (570 mg, 1.5 mmol) were added, and the mixture was stirred at room temperature for 2 h. The reaction mixture was added dropwise into water and extracted with ethyl acetate. The organic phase was dried over anhydrous sodium sulfate, filtered, and concentrated under reduced pressure. The residue was separated and purified by silica gel column (dichloromethane/methanol=20/1) to obtain Intermediate INT-23 with a yield of 70%. MS (ESI, positive ion) m/z: 430.3. [M+H]⁺.

### Preparation of Intermediate INT-24

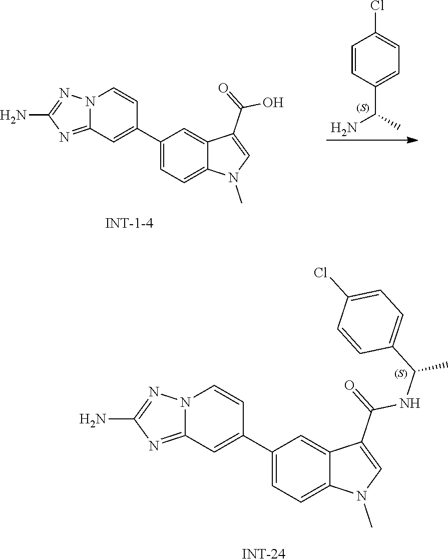

### Intermediate INT-24: Preparation of (S)-5-(2-amino-[1,2,4]triazolo[1,5-a]pyridin-7-yl)-N-(1-(4-chlorophenyl)ethyl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(4-chlorophenyl)ethan-1-amine (186 mg, 1.2 mmol) was added, and the mixture was stirred for reaction at room temperature for 2 h. The reaction mixture was added dropwise to water and stirred for 0.5 h. The mixture was filtered and dried to obtain Intermediate INT-24 with a yield of 70%. MS (ESI, positive ion) m/z: 445.1 [M+H]⁺.

### Preparation of Intermediate INT-25

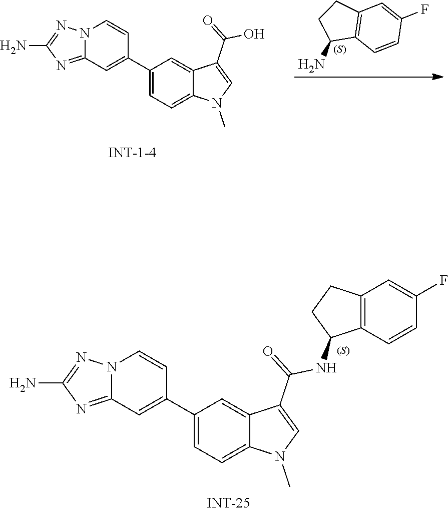

### Intermediate INT-25: Preparation of (S)-5-(2-amino-[1,2,4]triazolo[1,5-a]pyridin-7-yl)-N-(5-fluoro-2,3-dihydro-1H-inden-1-yl)-1-methyl-1H-indol-3-carboxamide

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-5-fluoro-2,3-dihydro-1H-inden-1-amine (181 mg, 1.2 mmol) was added, and the mixture was stirred for reaction at room temperature for 2 h; the reaction mixture was added dropwise into water and stirred for 0.5 h; the mixture was filtered and dried to obtain Intermediate INT-25 with a yield of 68%. MS (ESI, positive ion) m/z: 443.3 [M+H]⁺.

### Preparation of Intermediate INT-26

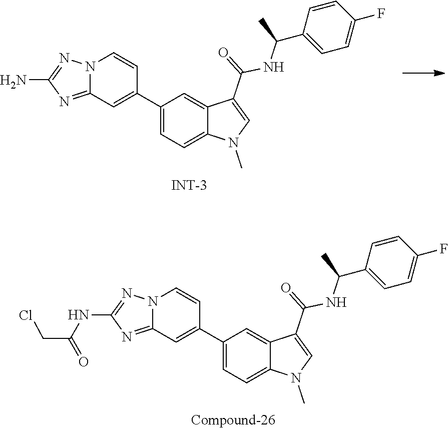

### Preparation of Intermediate INT-26

Intermediate INT-3 (42.8 g, 100 mmol) was dissolved in dichloromethane (1 L), chloroacetyl chloride (13.5 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Intermediate INT-26 with a yield of 82%. MS (ESI, positive ion) m/z: 505.2 [M+H]⁺.

### Preparation of Intermediate INT-27

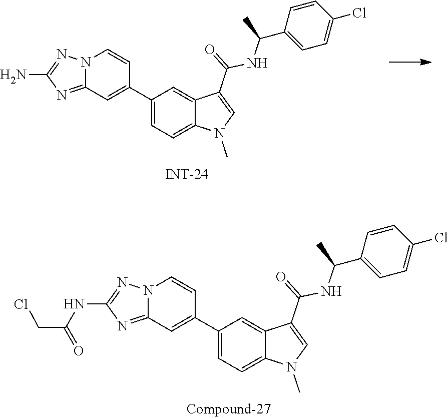

### Preparation of Intermediate INT-27

Intermediate INT-24 (44.4 g, 100 mmol) was dissolved in dichloromethane (1 L), chloroacetyl chloride (13.5 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Intermediate INT-27 with a yield of 78%. MS (ESI, positive ion) m/z: 521.2 [M+H]⁺.

### Preparation of Intermediate INT-28

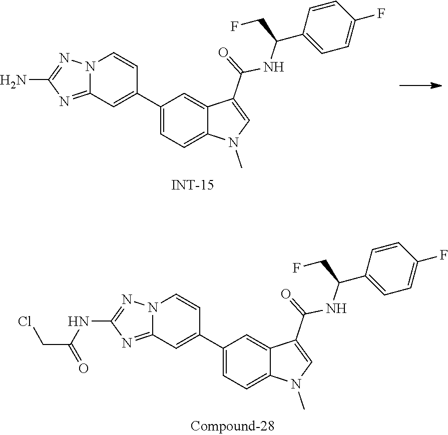

### Preparation of Intermediate INT-28

Intermediate INT-15 (44.6 g, 100 mmol) was dissolved in dichloromethane (1 L), chloroacetyl chloride (13.5 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Intermediate INT-28 with a yield of 75%. MS (ESI, positive ion) m/z: 523.2 [M+H]⁺.

### Preparation of Intermediate INT-29

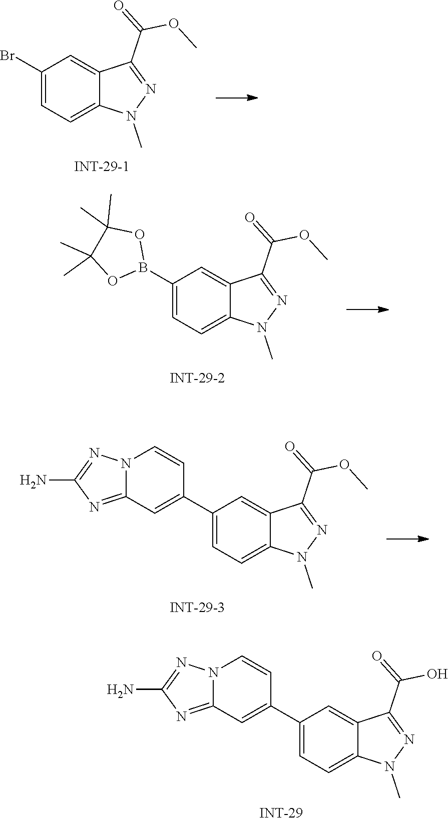

### Intermediate INT-29-2: Preparation of methyl 1-methyl-5-(4,4,5,5-tetramethyl-1,3,2-dioxaborolan-2-yl)-1H-indazole-3-carboxylate

Under an argon atmosphere, Intermediate INT-29-1 (1335 mg, 5.0 mmol) and bis(pinacolato) diboron (1651 mg, 6.5 mmol) were dissolved in 1,4-dioxane (20 mL), and potassium acetate (4.9 g, 10.0 mmol) and PdCl₂(dppf)₂ (160 mg) were added; three cycles of argon purging was performed, and the mixture was heated to 100° C. and stirred for 10 h. The reaction mixture was concentrated under reduced pressure and separated and purified by silica gel column (petroleum ether/ethyl acetate=5/1) to obtain Intermediate INT-29-2 with a yield of 68%. MS (ESI, positive ion) m/z: 317.3 [M+H]⁺.

### Intermediate INT-29-3: Preparation of 5-(2-amino-[1,2,4]triazolo[1,5-a]pyridin-7-yl)-1-methyl-1H-indazole-3-carboxylic acid methyl ester

Under an argon atmosphere, Intermediate INT-29-2 (1260 mg, 4.0 mmol) and 7-bromo-[1,2,4]triazolo[1,5-a]pyridin-2-amine (848 mg, 4.0 mmol) were dissolved in 1,4-dioxane (20 mL), potassium phosphate (1.7 g, 8.0 mmol), PdCl₂(dppf)₂ (120 mg), and water (2.0 mL) were added; three cycles of argon purging was performed, the mixture was heated to 95° C. and stirred for 8 h. The reaction mixture was concentrated under reduced pressure and separated and purified by silica gel column (dichloromethane/methanol=20/1) to obtain Intermediate INT-29-3 with a yield of 70%. MS (ESI, positive ion) m/z: 323.2 [M+H]⁺.

### Intermediate INT-29: Preparation of 5-(2-amino-[1,2,4]triazolo[1,5-a]pyridin-7-yl)-1-methyl-1H-indazole-3-carboxylic acid

Intermediate INT-29-3 (963 mg, 3.0 mmol) was dissolved in ethanol (3 mL), tetrahydrofuran (3 mL), and water (15 mL); sodium hydroxide (0.96 g, 24 mmol) was added; the mixture was heated to 75° C. and stirred for 5 h. The pH was adjusted to slightly acidic with dilute hydrochloric acid (1 M). The mixture was filtered and dried to obtain Intermediate INT-29 with a yield of 62%. MS (ESI, positive ion) m/z: 309.3 [M+H]⁺.

### Example 1: Preparation of Compound 1

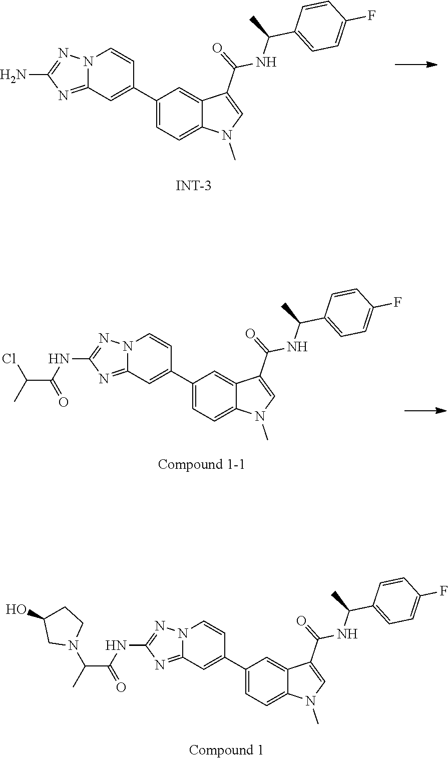 [SMILES: C[C@H](NC(=O)c1cn(C)c2ccc(-c3ccn4nc(N)nc4c3)cc12)c1ccc(F)cc1 ; "INT-3" ; CC(Cl)C(=O)Nc1nc2cc(-c3ccc4c(c3)c(C(=O)N[C@@H](C)c3ccc(F)cc3)cn4C)ccn2n1 ; CC(C(=O)Nc1nc2cc(-c3ccc4c(c3)c(C(=O)N[C@@H](C)c3ccc(F)cc3)cn4C)ccn2n1)N1CC[C@H](O)C1]

### Preparation of Compound 1-1

Intermediate INT-3 (214 mg, 0.5 mmol) was dissolved in pyridine (2 mL); 2-chloropropionic acid (65 mg, 0.6 mmol) and 1-propylphosphonic anhydride (316 mg, 1.0 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 1-1 with a yield of 61%. MS (ESI, positive ion) m/z: 519.2 [M+H]⁺.

### Preparation of Compound 1

Compound 1-1 (100 mg, 0.2 mmol) was dissolved in acetonitrile (1 mL); (S)-3-pyrrolidinol (43 mg, 0.5 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 1 with a yield of 45%. MS (ESI, positive ion) m/z: 570.2 [M+H]⁺.

¹H NMR (400 MHz, DMSO-d₆) δ 10.62 (s, 1H), 8.89 (dd, J=7.1, 0.8 Hz, 1H), 8.56 (d, J=1.9 Hz, 1H), 8.36 (d, J=8.0 Hz, 1H), 8.23 (s, 1H), 7.91 (dd, J=1.9, 0.9 Hz, 1H), 7.74 (dd, J=8.7, 1.9 Hz, 1H), 7.68 (d, J=8.6 Hz, 1H), 7.48 (dd, J=7.4, 2.0 Hz, 3H), 7.21-7.14 (m, 2H), 5.25-5.20 (m, 1H), 3.92 (s, 3H), 3.67 (m, 1H), 2.48-2.34 (m, 4H), 1.82-1.63 (m, 4H), 1.51 (d, J=7.1 Hz, 3H), 1.29 (d, J=7.4 Hz, 3H).

### Example 2: Preparation of Compound 2

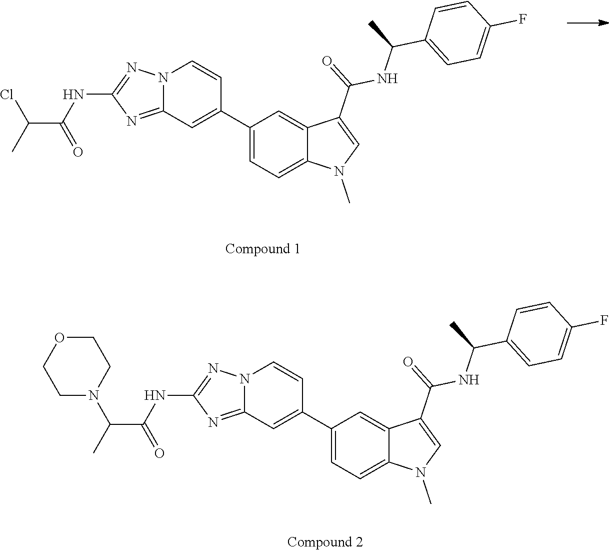 [SMILES: CC(Cl)C(=O)Nc1nc2cc(-c3ccc4c(c3)c(C(=O)N[C@@H](C)c3ccc(F)cc3)cn4C)ccn2n1 ; CC(C(=O)Nc1nc2cc(-c3ccc4c(c3)c(C(=O)N[C@@H](C)c3ccc(F)cc3)cn4C)ccn2n1)N1CCOCC1]

### Preparation of Compound 2

Compound 1-1 (100 mg, 0.2 mmol) was dissolved in acetonitrile (1 mL); morpholine (43 mg, 0.5 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 1 with a yield of 62%. MS (ESI, positive ion) m/z: 570.2 [M+H]⁺.

¹H NMR (400 MHz, DMSO-d₆) δ 10.62 (s, 1H), 8.89 (dd, J=7.1, 0.8 Hz, 1H), 8.56 (d, J=1.9 Hz, 1H), 8.36 (d, J=8.0 Hz, 1H), 8.23 (s, 1H), 7.91 (dd, J=1.9, 0.9 Hz, 1H), 7.74 (dd, J=8.7, 1.9 Hz, 1H), 7.68 (d, J=8.6 Hz, 1H), 7.48 (dd, J=7.4, 2.0 Hz, 3H), 7.21-7.14 (m, 2H), 5.25-5.20 (m, 1H), 3.92 (s, 3H), 3.67 (m, 1H), 3.48-3.34 (m, 4H), 2.82-2.63 (m, 4H), 1.51 (d, J=7.1 Hz, 3H), 1.29 (d, J=7.4 Hz, 3H).

### Example 3: Preparation of Compound 3

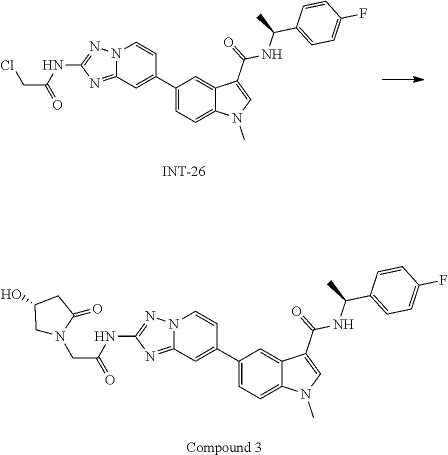 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5C[C@H](O)CC5=O)nc4c3)cc12)c1ccc(F)cc1 ; "Compound 3"]

### Preparation of Compound 3

(R)-4-hydroxy-2-pyrrolidone (101 mg, 1 mmol) was dissolved in dry tetrahydrofuran (2 mL), and sodium hydride (80 mg, 2 mmol) was added. After 5 min, Intermediate INT-26 (252 mg, 0.5 mmol) was added, and the mixture was stirred at room temperature for 3 h. The reaction mixture was quenched with water and extracted with ethyl acetate; the organic phase was concentrated under reduced pressure and purified by C-18 reverse phase silica gel column (acetonitrile/0.1% aqueous ammonium bicarbonate solution=60%) to obtain Compound 3 with a yield of 41%. MS (ESI, positive ion) m/z: 570.2 [M+H]⁺.

¹H NMR (400 MHz, DMSO-d₆) δ 10.62 (s, 1H), 8.89 (dd, J=7.1, 0.8 Hz, 1H), 8.56 (d, J=1.9 Hz, 1H), 8.36 (d, J=8.0 Hz, 1H), 8.23 (s, 1H), 7.91 (dd, J=1.9, 0.9 Hz, 1H), 7.74 (dd, J=8.7, 1.9 Hz, 1H), 7.68 (d, J=8.6 Hz, 1H), 7.48 (dd, J=7.4, 2.0 Hz, 3H), 7.21-7.14 (m, 2H), 5.40 (s, 1H), 5.25-5.20 (m, 1H), 3.92 (s, 3H), 3.56-3.43 (m, 3H), 3.67-3.42 (m, 2H), 2.50-2.25 (m, 2H), 1.51 (d, J=7.1 Hz, 3H).

### Example 4: Preparation of Compound 4

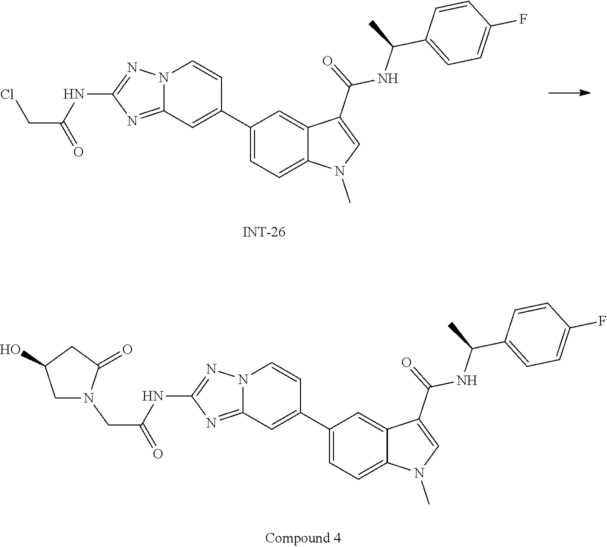 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5C[C@@H](O)CC5=O)nc4c3)cc12)c1ccc(F)cc1]

### Preparation of Compound 4

(S)-4-hydroxy-2-pyrrolidone (101 mg, 1 mmol) was dissolved in dry tetrahydrofuran (2 mL), and sodium hydride (80 mg, 2 mmol) was added. After 5 min, Intermediate INT-26 (252 mg, 0.5 mmol) was added, and the mixture was stirred at room temperature for 3 h. The reaction mixture was quenched with water and extracted with ethyl acetate; the organic phase was concentrated under reduced pressure and purified by C-18 reverse phase silica gel column (acetonitrile/0.1% aqueous ammonium bicarbonate solution=60%) to obtain Compound 4 with a yield of 43%. MS (ESI, positive ion) m/z: 570.2 [M+H]⁺.

¹H NMR (400 MHz, DMSO-d₆) δ 10.62 (s, 1H), 8.89 (dd, J=7.1, 0.8 Hz, 1H), 8.56 (d, J=1.9 Hz, 1H), 8.36 (d, J=8.0 Hz, 1H), 8.23 (s, 1H), 7.91 (dd, J=1.9, 0.9 Hz, 1H), 7.74 (dd, J=8.7, 1.9 Hz, 1H), 7.68 (d, J=8.6 Hz, 1H), 7.48 (dd, J=7.4, 2.0 Hz, 3H), 7.21-7.14 (m, 2H), 5.40 (s, 1H), 5.25-5.20 (m, 1H), 3.92 (s, 3H), 3.56-3.43 (m, 3H), 3.67-3.42 (m, 2H), 2.50-2.25 (m, 2H), 1.51 (d, J=7.1 Hz, 3H).

### Example 5: Preparation of Compound 5

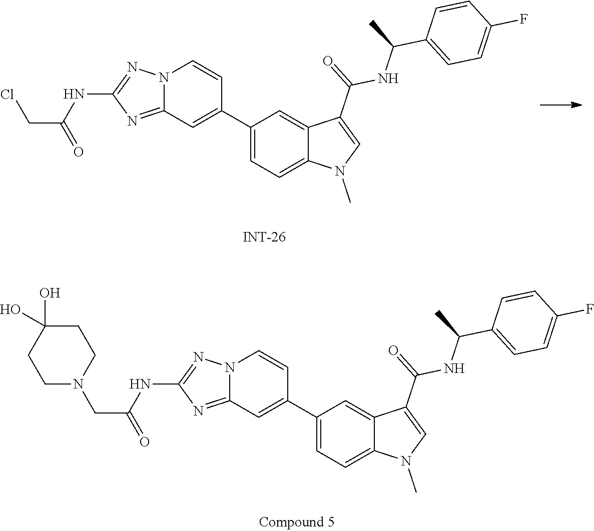 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC(O)(O)CC5)nc4c3)cc12)c1ccc(F)cc1]

### Preparation of Compound 5

Intermediate INT-26 (101 mg, 0.2 mmol) was dissolved in acetonitrile (1 mL); 4,4-piperidinediol hydrochloride (60 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 5 with a yield of 32%. MS (ESI, positive ion) m/z: 586.2 [M+H]⁺.

¹H NMR (400 MHz, DMSO-d₆) δ 10.62 (s, 1H), 8.89 (dd, J=7.1, 0.8 Hz, 1H), 8.56 (d, J=1.9 Hz, 1H), 8.36 (d, J=8.0 Hz, 1H), 8.23 (s, 1H), 7.91 (dd, J=1.9, 0.9 Hz, 1H), 7.74 (dd, J=8.7, 1.9 Hz, 1H), 7.68 (d, J=8.6 Hz, 1H), 7.48 (dd, J=7.4, 2.0 Hz, 3H), 7.21-7.14 (m, 2H), 5.25-5.20 (m, 1H), 3.92 (s, 3H), 3.46 (s, 2H), 2.92 (t, J=6.0 Hz, 4H), 2.55 (d, J=6.8 Hz, 2H), 2.48-2.34 (m, 4H), 1.51 (d, J=7.1 Hz, 3H).

### Example 6: Preparation of Compound 6

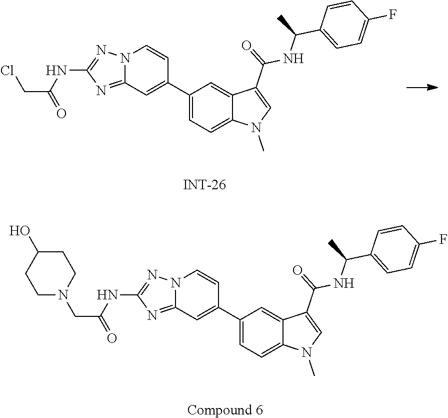 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC(O)CC5)nc4c3)cc12)c1ccc(F)cc1 ; "Compound 6"]

### Preparation of Compound 6

Intermediate INT-26 (101 mg, 0.2 mmol) was dissolved in acetonitrile (1 mL); 4-piperidinol (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 6 with a yield of 42%. MS (ESI, positive ion) m/z: 570.2 [M+H]⁺.

¹H NMR (400 MHz, DMSO-d6) δ 10.38 (s, 1H), 8.89 (d, J=7.1 Hz, 1H), 8.55 (d, J=1.8 Hz, 1H), 8.36 (d, J=8.0 Hz, 1H), 8.22 (s, 1H), 7.90 (dd, J=1.9, 0.9 Hz, 1H), 7.73 (d, J=1.9 Hz, 1H), 7.68 (d, J=8.7 Hz, 1H), 7.50-7.46 (m, 3H), 7.17 (t, J=8.9 Hz, 2H), 5.28-5.18 (m, 1H), 4.58 (d, J=4.2 Hz, 1H), 3.92 (s, 3H), 3.55-3.45 (m, 1H), 3.21 (s, 2H), 2.85-2.76 (m, 2H), 2.30 (t, J=10.2 Hz, 2H), 1.76 (d, J=10.9 Hz, 2H), 1.51 (d, J=7.0 Hz, 5H).

### Example 7: Preparation of Compound 7

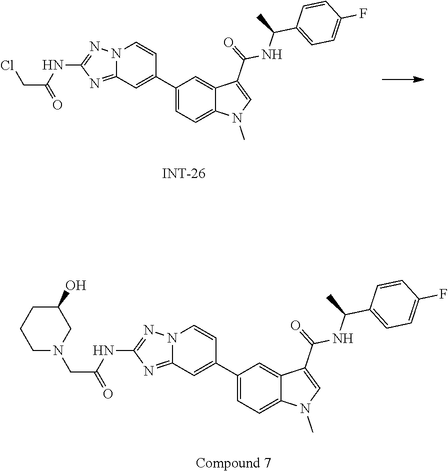 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC[C@@H](O)C5)nc4c3)cc12)c1ccc(F)cc1 ; "Compound 7"]

### Preparation of Compound 7

Intermediate INT-26 (101 mg, 0.2 mmol) was dissolved in acetonitrile (1 mL); S-3-hydroxypiperidine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 7 with a yield of 46%. MS (ESI, positive ion) m/z: 570.2 [M+H]⁺.

¹H NMR (400 MHz, DMSO-d₆) δ 10.42 (s, 1H), 8.89 (dd, J=7.1, 0.8 Hz, 1H), 8.55 (d, J=1.8 Hz, 1H), 8.36 (d, J=8.0 Hz, 1H), 8.22 (s, 1H), 7.90 (dd, J=1.9, 0.9 Hz, 1H), 7.74 (dd, J=8.7, 1.8 Hz, 1H), 7.68 (d, J=8.6 Hz, 1H), 7.50-7.46 (m, 3H), 7.17 (t, J=8.9 Hz, 2H), 5.28-5.18 (m, 1H), 4.74 (d, J=5.5 Hz, 1H), 3.92 (s, 3H), 3.58 (dd, J=9.0, 4.6 Hz, 1H), 3.22 (d, J=8.7 Hz, 2H), 2.83 (d, J=9.6 Hz, 1H), 2.66 (dd, J=15.1, 9.2 Hz, 1H), 2.23 (t, J=10.2 Hz, 1H), 2.12 (t, J=9.4 Hz, 1H), 1.80-1.66 (m, 2H), 1.50 (d, J=7.0 Hz, 4H), 1.18 (q, J=9.0, 8.3 Hz, 1H).

### Example 8: Preparation of Compound 8

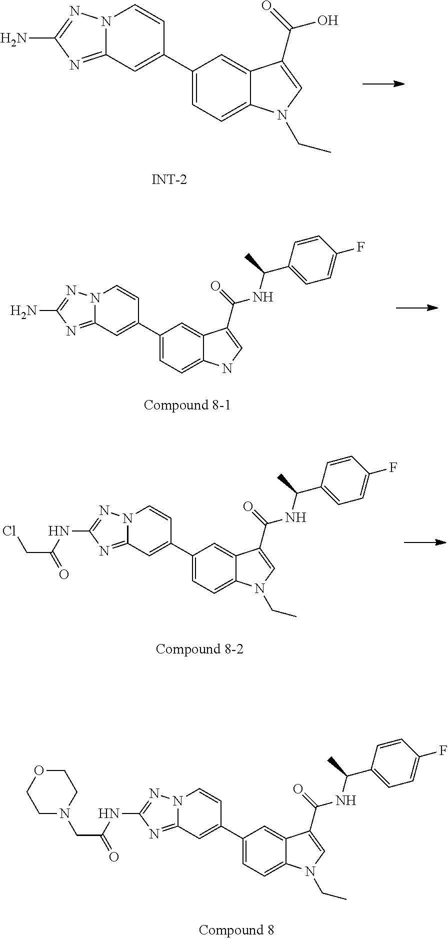 [SMILES: CCn1cc(C(=O)O)c2cc(-c3ccn4nc(N)nc4c3)ccc21 ; "INT-2" ; C[C@H](NC(=O)c1c[nH]c2ccc(-c3ccn4nc(N)nc4c3)cc12)c1ccc(F)cc1 ; CCn1cc(C(=O)N[C@@H](C)c2ccc(F)cc2)c2cc(-c3ccn4nc(NC(=O)CCl)nc4c3)ccc21 ; CCn1cc(C(=O)N[C@@H](C)c2ccc(F)cc2)c2cc(-c3ccn4nc(NC(=O)CN5CCOCC5)nc4c3)ccc21]

### Preparation of Compound 8-1

Intermediate INT-2 (321 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(4-fluorophenyl)ethan-1-amine (167 mg, 1.2 mmol) was added, and the mixture was stirred for reaction at room temperature for 2 h. The reaction mixture was added dropwise to water and stirred for 0.5 h. The mixture was filtered and dried to obtain Compound 8-1 with a yield of 73%. MS (ESI, positive ion) m/z: 443.3 [M+H]⁺.

### Preparation of Compound 8-2

Compound 8-1 (442 mg, 1.0 mmol) was dissolved in dichloromethane (10 mL); chloroacetyl chloride (135 mg, 1.2 mmol) and N,N-diisopropylethylamine (258 mg, 2.0 mmol) were added, and the mixture was stirred at room temperature for 2 h. Petroleum ether (30 mL) was added to the reaction mixture for crystallization; the solid was filtered with suction, washed with water, and dried to obtain Compound 8-2 with a yield of 79%. MS (ESI, positive ion) m/z: 519.2 [M+H]⁺.

### Preparation of Compound 8

Compound 8-2 (104 mg, 0.2 mmol) was dissolved in acetonitrile (1 mL); morpholine (43 mg, 0.5 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 8 with a yield of 52%. MS (ESI, positive ion) m/z: 570.2 [M+H]⁺.

¹H NMR (400 MHz, DMSO-d₆) δ 10.51 (s, 1H), 8.89 (d, J=7.1 Hz, 1H), 8.56 (s, 1H), 8.35 (d, J=8.0 Hz, 1H), 8.30 (s, 1H), 7.89 (d, J=1.9 Hz, 1H), 7.72 (d, J=1.3 Hz, 2H), 7.48 (dd, J=8.4, 5.4 Hz, 3H), 7.20-7.15 (m, 2H), 5.23 (t, J=7.3 Hz, 1H), 4.32 (q, J=7.3 Hz, 2H), 3.65 (t, J=4.6 Hz, 4H), 2.57 (t, J=4.4 Hz, 4H), 1.52-1.46 (m, 6H).

### Example 9: Preparation of Compound 9

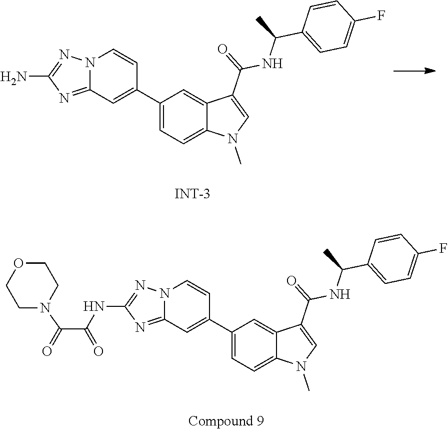 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)C(=O)N5CCOCC5)nc4c3)cc12)c1ccc(F)cc1]

### Preparation of Compound 9

Intermediate INT-3 (85 mg, 0.2 mmol) was dissolved in pyridine (1 mL), 2-morpholin-4-yl-2-oxo-acetic acid (48 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 9 with a yield of 56%. MS (ESI, positive ion) m/z: 570.2 [M+H]⁺.

¹H NMR (400 MHz, DMSO-d₆) δ 11.60 (s, 1H), 8.89 (d, J=7.3 Hz, 1H), 8.55 (d, J=1.8 Hz, 1H), 8.36 (d, J=8.0 Hz, 1H), 8.22 (s, 1H), 7.93 (s, 1H), 7.74 (dd, J=8.5, 1.9 Hz, 1H), 7.66 (d, J=8.6 Hz, 1H), 7.48 (dtd, J=12.1, 6.4, 5.7, 2.0 Hz, 3H), 7.19-7.13 (m, 2H), 5.22 (t, J=7.3 Hz, 1H), 3.91 (s, 3H), 3.70 (d, J=22.8 Hz, 4H), 3.56 (t, J=4.9 Hz, 2H), 3.46 (s, 2H), 1.50 (d, J=7.0 Hz, 3H).

### Example 10: Preparation of Compound 10

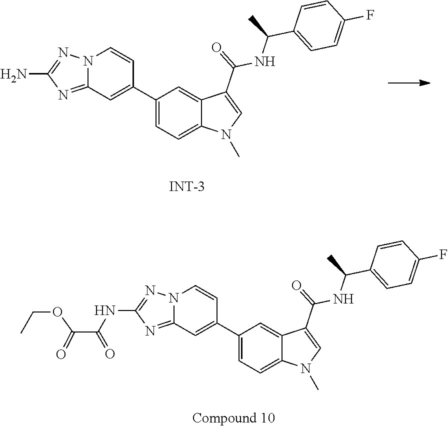 [SMILES: C[C@H](NC(=O)c1cn(C)c2ccc(-c3ccn4nc(N)nc4c3)cc12)c1ccc(F)cc1 ; "INT-3" ; CCOC(=O)C(=O)Nc1nc2cc(-c3ccc4c(c3)c(C(=O)N[C@@H](C)c3ccc(F)cc3)cn4C)ccn2n1]

### Preparation of Compound 10

Intermediate INT-3 (85 mg, 0.2 mmol) was dissolved in pyridine (1 mL), monoethyl oxalate (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 10 with a yield of 46%. MS (ESI, positive ion) m/z: 529.2 [M+H]⁺.

¹H NMR (400 MHz, DMSO-d₆) δ 11.76-11.52 (m, 1H), 8.87 (d, J=6.9 Hz, 1H), 8.53 (d, J=1.9 Hz, 1H), 8.34 (d, J=8.0 Hz, 1H), 8.19 (s, 1H), 7.91 (s, 1H), 7.69 (dd, J=8.7, 1.8 Hz, 1H), 7.61 (d, J=8.6 Hz, 1H), 7.49-7.41 (m, 3H), 7.15-7.09 (m, 2H), 5.19 (t, J=7.3 Hz, 1H), 4.30 (q, J=7.1 Hz, 2H), 3.86 (s, 3H), 1.46 (d, J=7.0 Hz, 3H), 1.28 (t, J=7.0 Hz, 3H).

### Example 11: Preparation of Compound 11

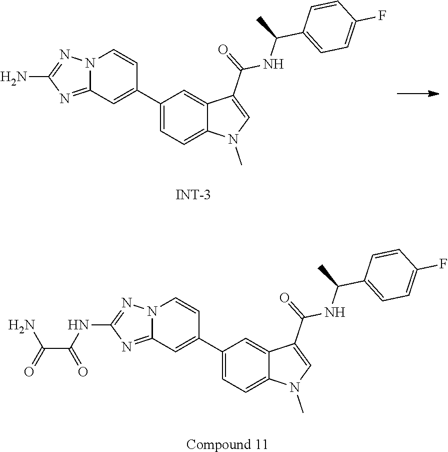 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)C(N)=O)nc4c3)cc12)c1ccc(F)cc1 ; "Compound 11"]

### Preparation of Compound 11

Intermediate INT-3 (85 mg, 0.2 mmol) was dissolved in pyridine (1 mL); oxamic acid (27 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 11 with a yield of 51%. MS (ESI, positive ion) m/z: 500.2 [M+H]⁺.

¹H NMR (400 MHz, DMSO-d₆) δ 10.87 (s, 1H), 8.93 (d, J=7.2 Hz, 1H), 8.56 (d, J=1.8 Hz, 1H), 8.36 (d, J=7.8 Hz, 2H), 8.22 (s, 1H), 8.08-7.95 (m, 2H), 7.76 (dd, J=8.7, 1.9 Hz, 1H), 7.68 (d, J=8.6 Hz, 1H), 7.54-7.45 (m, 3H), 7.19-7.14 (m, 2H), 5.27-5.18 (m, 1H), 3.91 (s, 3H), 1.50 (d, J=7.0 Hz, 3H).

### Example 12: Preparation of Compound 12

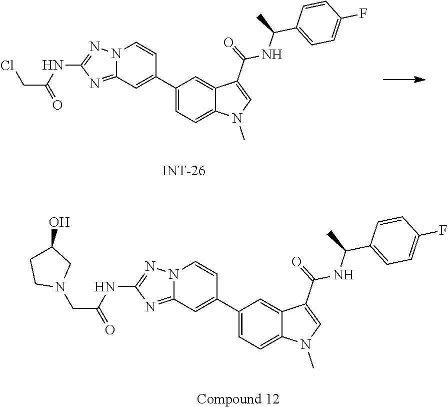 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@@H](O)C5)nc4c3)cc12)c1ccc(F)cc1 ; "Compound 12"]

### Preparation of Compound 12

Intermediate INT-26 (101 mg, 0.2 mmol) was dissolved in acetonitrile (1 mL); (R)-3-pyrrolidine (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 12 with a yield of 46%. MS (ESI, positive ion) m/z: 556.2 [M+H]⁺.

¹H NMR (400 MHz, DMSO-d₆) δ 10.44 (s, 1H), 8.89 (d, J=7.1 Hz, 1H), 8.55 (d, J=1.8 Hz, 1H), 8.36 (d, J=8.0 Hz, 1H), 8.23 (s, 1H), 7.90 (d, J=1.9 Hz, 1H), 7.74 (dd, J=8.7, 1.9 Hz, 1H), 7.67 (d, J=8.6 Hz, 1H), 7.50-7.45 (m, 3H), 7.21-7.14 (m, 2H), 5.22 (p, J=7.1 Hz, 1H), 4.83 (d, J=5.5 Hz, 1H), 4.23 (s, 1H), 3.91 (s, 3H), 3.36 (s, 2H), 2.87-2.79 (m, 2H), 2.60-2.55 (m, 2H), 2.06 (dq, J=14.0, 7.2 Hz, 1H), 1.69-1.59 (m, 1H), 1.50 (d, J=7.0 Hz, 3H).

### Example 13: Preparation of Compound 13

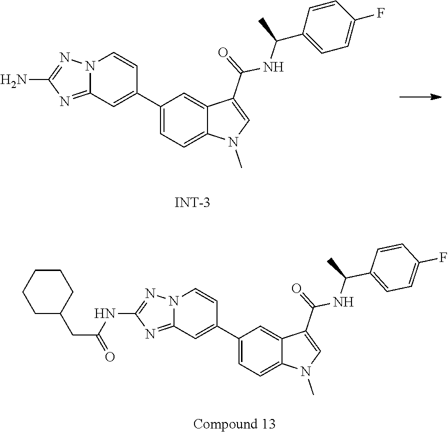 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)CC5CCCCC5)nc4c3)cc12)c1ccc(F)cc1 ; "Compound 13"]

### Preparation of Compound 13

Intermediate INT-3 (85 mg, 0.2 mmol) was dissolved in pyridine (1 mL); cyclohexylacetic acid (42 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 13 with a yield of 61%. MS (ESI, positive ion) m/z: 553.2 [M+H]⁺.

¹H NMR (400 MHz, DMSO-d₆) δ 10.78 (s, 1H), 8.86 (d, J=7.1 Hz, 1H), 8.55 (d, J=1.8 Hz, 1H), 8.37 (d, J=8.0 Hz, 1H), 8.23 (s, 1H), 7.88 (d, J=1.9 Hz, 1H), 7.73 (dd, J=8.6, 1.9 Hz, 1H), 7.66 (d, J=8.6 Hz, 1H), 7.47 (ddd, J=8.6, 5.5, 2.4 Hz, 3H), 7.17 (t, J=8.9 Hz, 2H), 5.27-5.17 (m, 1H), 3.91 (s, 3H), 3.85 (ddd, J=11.3, 4.4, 1.9 Hz, 2H), 3.31 (dd, J=11.6, 2.1 Hz, 2H), 2.40 (s, 1H), 2.04 (dt, J=7.4, 3.5 Hz, 1H), 1.67-1.59 (m, 2H), 1.50 (d, J=7.1 Hz, 3H), 1.28 (tt, J=12.1, 6.0 Hz, 2H).

### Example 14: Preparation of Compound 14

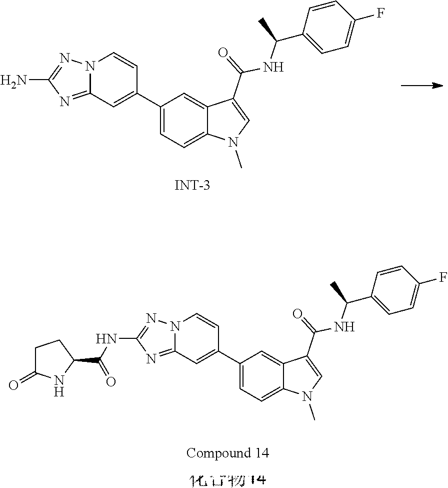

### Preparation of Compound 14

Intermediate INT-3 (85 mg, 0.2 mmol) was dissolved in pyridine (1 mL); L-pyroglutamic acid (39 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 14 with a yield of 53%. MS (ESI, positive ion) m/z: 540.2 [M+H]⁺.

¹H NMR (400 MHz, DMSO-d₆) δ 8.42 (t, J=1.2 Hz, 1H), 8.31 (d, J=8.0 Hz, 1H), 8.19 (s, 1H), 7.88 (d, J=1.6 Hz, 1H), 7.66-7.59 (m, 4H), 7.51-7.42 (m, 2H), 7.22-7.11 (m, 2H), 5.22 (p, J=7.1 Hz, 1H), 4.11 (m, 1H), 3.90 (s, 3H), 2.46-2.08 (m, 4H), 1.50 (d, J=7.0 Hz, 3H).

### Example 15: Preparation of Compound 15

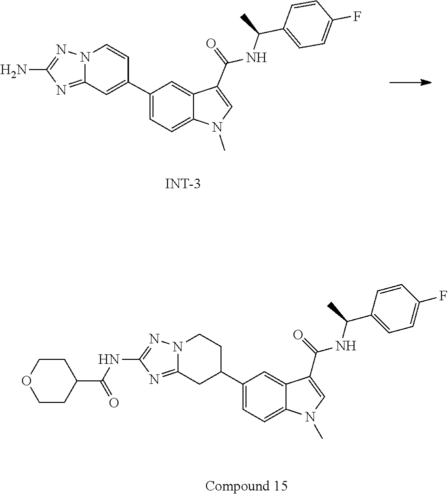 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(C3CCn4nc(NC(=O)C5CCOCC5)nc4C3)cc12)c1ccc(F)cc1]

### Preparation of Compound 15

Intermediate INT-3 (85 mg, 0.2 mmol) was dissolved in pyridine (1 mL); cyclohexanecarboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 15 with a yield of 51%. MS (ESI, positive ion) m/z: 540.2 [M+H]⁺.

¹H NMR (400 MHz, DMSO-d₆) δ 8.42 (t, J=1.2 Hz, 1H), 8.31 (d, J=8.0 Hz, 1H), 8.19 (s, 1H), 7.88 (d, J=1.6 Hz, 1H), 7.66-7.59 (m, 3H), 7.51-7.42 (m, 2H), 7.22-7.11 (m, 2H), 5.22 (p, J=7.1 Hz, 1H), 3.90 (s, 3H), 3.63 (t, J=4.6 Hz, 4H), 2.60-2.55 (m, 4H), 1.50 (d, J=7.0 Hz, 3H).

### Example 16: Preparation of Compound 16

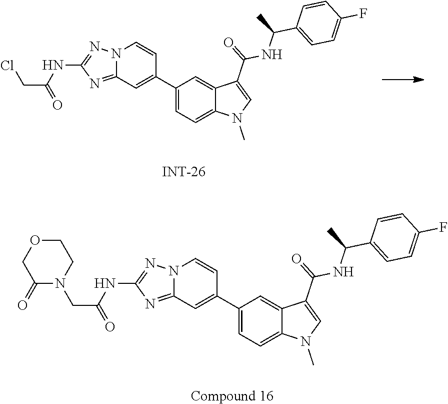 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOCC5=O)nc4c3)cc12)c1ccc(F)cc1 ; "Compound 16"]

### Preparation of Compound 16

3-morpholinone (101 mg, 1 mmol) was dissolved in dry tetrahydrofuran (2 mL), and sodium hydride (80 mg, 2 mmol) was added. After 5 min, Intermediate INT-26 (252 mg, 0.5 mmol) was added, and the mixture was stirred at room temperature for 3 h. The reaction mixture was quenched with water and extracted with ethyl acetate; the organic phase was concentrated under reduced pressure and purified by C-18 reverse phase silica gel column (acetonitrile/0.1% aqueous ammonium bicarbonate solution=60%) to obtain Compound 16 with a yield of 33%. MS (ESI, positive ion) m/z: 570.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 4.97-4.82 (m, 1H), 4.30 (s, 2H), 3.91 (s, 3H), 3.65-3.52 (m, 2H), 2.57 (m, 2H), 2.35 (s, 2H), 1.50 (d, J=7.1 Hz, 3H), 1.25 (s, 6H).

### Example 17: Preparation of Compound 17

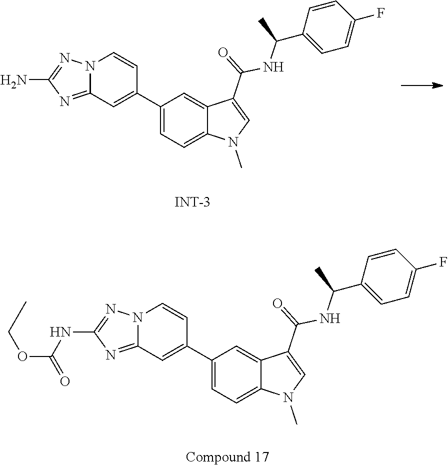

### Preparation of Compound 17

Intermediate INT-3 (43 mg, 0.10 mmol) was dissolved in dichloromethane (1 mL); ethyl chloroformate (13 mg, 0.12 mmol) and N,N-diisopropylethylamine (26 mg, 0.20 mmol) were added, and the mixture was stirred at room temperature for 2 h. Petroleum ether (3 mL) was added to the reaction mixture for crystallization; the solid was filtered with suction, washed with water, and dried to obtain Compound 17 with a yield of 82%. MS (ESI, positive ion) m/z: 501.2 [M+H]⁺.

¹H NMR (400 MHz, DMSO-d6) δ 10.46 (s, 1H), 8.83 (d, J=7.1 Hz, 1H), 8.52 (d, J=1.8 Hz, 1H), 8.34 (d, J=8.0 Hz, 1H), 8.20 (s, 1H), 7.85 (d, J=1.9 Hz, 1H), 7.71 (dd, J=8.6, 1.9 Hz, 1H), 7.65 (d, J=8.7 Hz, 1H), 7.49-7.37 (m, 3H), 7.19-7.10 (m, 2H), 5.25-5.15 (m, 1H), 4.16 (q, J=7.1 Hz, 2H), 1.48 (d, J=7.1 Hz, 3H), 1.25 (t, J=7.1 Hz, 3H).

### Example 18: Preparation of Compound 18

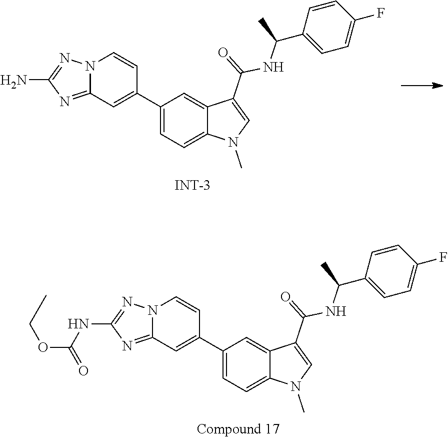

### Preparation of Compound 18

Intermediate INT-26 (101 mg, 0.2 mmol) was dissolved in acetonitrile (1 mL); 2,2-dimethylmorpholine (45 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 18 with a yield of 66%. MS (ESI, positive ion) m/z: 584.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 4.97-4.82 (m, 1H), 3.91 (s, 3H), 3.65-3.52 (m, 2H), 3.25 (s, 2H), 2.57 (m, 2H), 2.35 (s, 2H), 1.50 (d, J=7.1 Hz, 3H), 1.25 (s, 6H).

### Example 19: Preparation of Compound 19

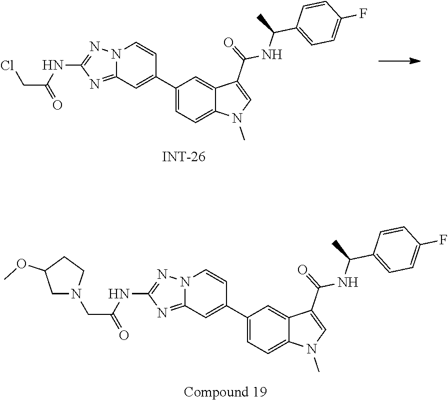 [SMILES: C[C@H](NC(=O)c1cn(C)c2ccc(-c3ccn4nc(NC(=O)CCl)nc4c3)cc12)c1ccc(F)cc1 ; "INT-26" ; COC1CCN(CC(=O)Nc2nc3cc(-c4ccc5c(c4)c(C(=O)N[C@@H](C)c4ccc(F)cc4)cn5C)ccn3n2)C1]

### Preparation of Compound 19

Intermediate INT-26 (101 mg, 0.2 mmol) was dissolved in acetonitrile (1 mL); 2-methoxypyrrolidine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 19 with a yield of 76%. MS (ESI, positive ion) m/z: 570.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 4.97-4.82 (m, 1H), 3.91 (s, 3H), 3.42-3.38 (m, 4H), 3.25 (s, 2H), 2.62-2.32 (m, 4H), 1.82-1.64 (m, 2H), 1.50 (d, J=7.1 Hz, 3H).

### Example 20: Preparation of Compound 20

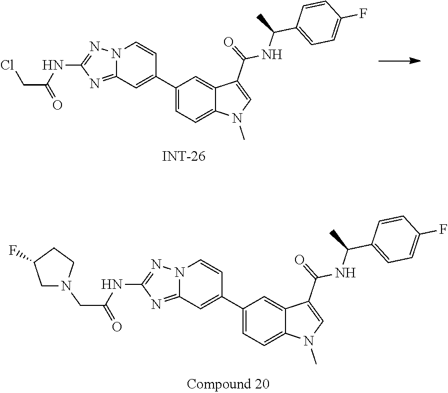 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@@H](F)C5)nc4c3)cc12)c1ccc(F)cc1]

### Preparation of Compound 20

Intermediate INT-26 (101 mg, 0.2 mmol) was dissolved in acetonitrile (1 mL); 3-(S)-pyrrolidinone (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 20 with a yield of 76%. MS (ESI, positive ion) m/z: 558.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 4.97-4.82 (m, 1H), 3.91 (s, 3H), 3.42-3.38 (m, 1H), 3.25 (s, 2H), 2.62-2.32 (m, 4H), 1.82-1.64 (m, 2H), 1.50 (d, J=7.1 Hz, 3H).

### Example 21: Preparation of Compound 21

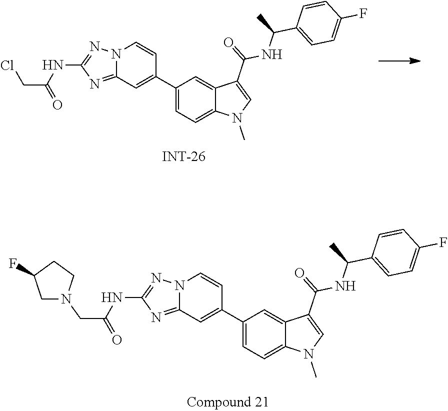 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@H](F)C5)nc4c3)cc12)c1ccc(F)cc1]

### Preparation of Compound 21

Intermediate INT-26 (101 mg, 0.2 mmol) was dissolved in acetonitrile (1 mL); 3-(R)-pyrrolidinone (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 21 with a yield of 66%. MS (ESI, positive ion) m/z: 558.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 4.97-4.82 (m, 1H), 3.91 (s, 3H), 3.42-3.38 (m, 1H), 3.25 (s, 2H), 2.62-2.32 (m, 4H), 1.82-1.64 (m, 2H), 1.50 (d, J=7.1 Hz, 3H).

### Example 22: Preparation of Compound 22

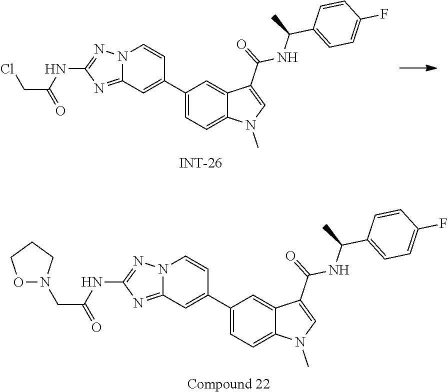 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCCO5)nc4c3)cc12)c1ccc(F)cc1 ; "Compound 22"]

### Preparation of Compound 22

Intermediate INT-26 (101 mg, 0.2 mmol) was dissolved in acetonitrile (1 mL); isoxazolidine (29 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 22 with a yield of 56%. MS (ESI, positive ion) m/z: 541.2 [M+H]⁺.

¹H NMR (400 MHz, DMSO-d₆) δ 8.56 (d, J=1.9 Hz, 1H), 8.37 (d, J=8.0 Hz, 1H), 8.23 (s, 1H), 7.98 (s, 1H), 7.78-7.66 (m, 3H), 7.53 (dd, J=7.2, 2.0 Hz, 1H), 7.47 (dd, J=8.5, 5.6 Hz, 3H), 7.18 (d, J=8.9 Hz, 2H), 5.27-5.17 (m, 1H), 4.07-3.98 (m, 1H), 3.91 (s, 3H), 3.70 (s, 2H), 2.01 (s, 2H), 1.57-1.54 (m, 1H), 1.50 (d, J=7.1 Hz, 3H), 0.88 (d, J=2.1 Hz, 2H).

### Example 23: Preparation of Compound 23

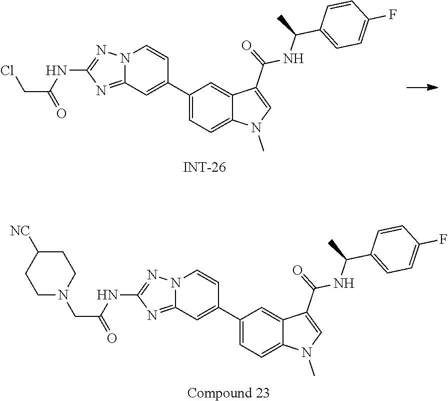 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC(C#N)CC5)nc4c3)cc12)c1ccc(F)cc1 ; "Compound 23"]

### Preparation of Compound 23

Intermediate INT-26 (101 mg, 0.2 mmol) was dissolved in acetonitrile (1 mL); 4-cyanopiperidine (44 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 23 with a yield of 66%. MS (ESI, positive ion) m/z: 579.2 [M+H]⁺.

¹H NMR (400 MHz, DMSO) δ 10.52 (s, 1H), 8.89 (d, J=7.1 Hz, 1H), 8.55 (d, J=1.4 Hz, 1H), 8.39 (d, J=8.0 Hz, 1H), 8.23 (s, 1H), 7.90 (d, J=1.1 Hz, 1H), 7.78-7.64 (m, 2H), 7.51-7.43 (m, 3H), 7.17 (t, J=8.9 Hz, 2H), 5.22 (p, J=6.9 Hz, 1H), 3.91 (s, 3H), 3.26 (d, J=11.5 Hz, 2H), 2.97-2.86 (m, 1H), 2.77-2.62 (m, 2H), 2.48 (s, 2H), 2.01-1.84 (m, 2H), 1.79 (ddd, J=12.9, 8.4, 4.0 Hz, 2H), 1.50 (d, J=7.0 Hz, 3H).

### Example 24: Preparation of Compound 24

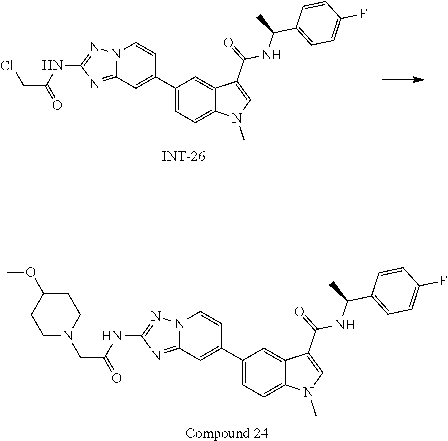 [SMILES: C[C@H](NC(=O)c1cn(C)c2ccc(-c3ccn4nc(NC(=O)CCl)nc4c3)cc12)c1ccc(F)cc1 ; "INT-26" ; COC1CCN(CC(=O)Nc2nc3cc(-c4ccc5c(c4)c(C(=O)N[C@@H](C)c4ccc(F)cc4)cn5C)ccn3n2)CC1 ; "Compound 24"]

### Preparation of Compound 24

Intermediate INT-26 (101 mg, 0.2 mmol) was dissolved in acetonitrile (1 mL); 4-methoxypiperidine (45 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 24 with a yield of 76%. MS (ESI, positive ion) m/z: 584.2 [M+H]⁺.

¹H NMR (400 MHz, DMSO) δ 10.44 (s, 1H), 8.89 (d, J=7.1 Hz, 1H), 8.55 (d, J=1.5 Hz, 1H), 8.39 (d, J=8.0 Hz, 1H), 8.23 (s, 1H), 7.90 (d, J=1.2 Hz, 1H), 7.78-7.63 (m, 2H), 7.52-7.43 (m, 3H), 7.26-7.11 (m, 2H), 5.22 (p, J=7.1 Hz, 1H), 3.91 (s, 3H), 3.38 (s, 3H), 3.23 (s, 3H), 2.77 (dd, J=13.7, 8.1 Hz, 2H), 2.38-2.29 (m, 2H), 1.87 (d, J=9.7 Hz, 2H), 1.50 (d, J=7.0 Hz, 5H).

### Example 25: Preparation of Compound 25

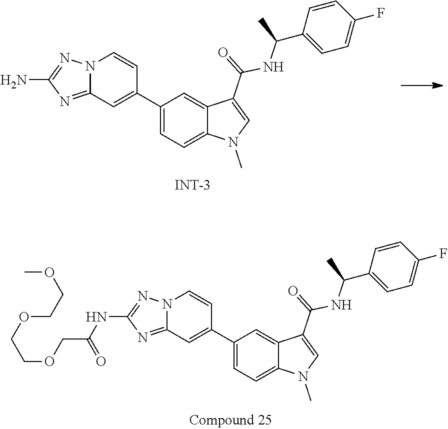

### Preparation of Compound 25

Intermediate INT-3 (85 mg, 0.2 mmol) was dissolved in pyridine (1 mL); 2-(2-(2-methoxyethoxy)ethoxy)acetic acid (53 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 25 with a yield of 51%. MS (ESI, positive ion) m/z: 589.2 [M+H]⁺.

¹H NMR (400 MHz, DMSO) δ 10.56 (s, 1H), 8.90 (d, J=7.1 Hz, 1H), 8.56 (d, J=1.2 Hz, 1H), 8.39 (d, J=8.0 Hz, 1H), 8.23 (s, 1H), 7.91 (s, 1H), 7.79-7.64 (m, 2H), 7.47 (dd, J=8.2, 5.9 Hz, 3H), 7.17 (t, J=8.9 Hz, 2H), 5.22 (p, J=7.1 Hz, 1H), 4.37-4.19 (m, 2H), 3.91 (s, 3H), 3.72-3.66 (m, 2H), 3.64-3.57 (m, 4H), 3.49 (dd, J=5.7, 3.7 Hz, 2H), 3.26 (s, 3H), 1.50 (d, J=7.0 Hz, 3H).

### Example 26: Preparation of Compound 26

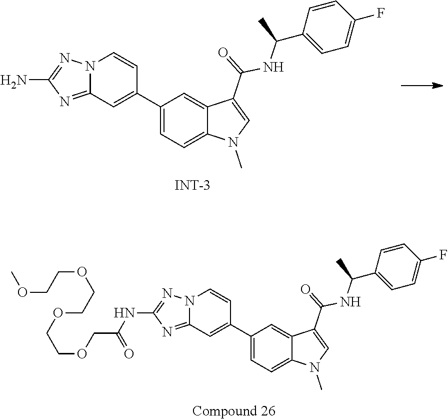

### Preparation of Compound 26

Intermediate INT-3 (85 mg, 0.2 mmol) was dissolved in pyridine (1 mL); triethylene glycol monomethyl ether acetate (66 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 26 with a yield of 48%. MS (ESI, positive ion) m/z: 633.2 [M+H]⁺.

¹H NMR (400 MHz, DMSO) δ 10.54 (s, 1H), 8.90 (d, J=7.1 Hz, 1H), 8.55 (d, J=1.2 Hz, 1H), 8.38 (d, J=8.0 Hz, 1H), 8.23 (s, 1H), 7.93 (d, J=15.9 Hz, 1H), 7.78-7.64 (m, 2H), 7.47 (dd, J=8.1, 6.0 Hz, 3H), 7.17 (t, J=8.9 Hz, 2H), 5.22 (p, J=7.0 Hz, 1H), 4.25 (s, 2H), 3.91 (s, 3H), 3.70 (dd, J=5.8, 3.3 Hz, 2H), 3.62 (dd, J=5.6, 3.4 Hz, 2H), 3.58 (s, 4H), 3.53 (dd, J=5.8, 3.6 Hz, 2H), 3.43 (dd, J=5.7, 3.8 Hz, 2H), 3.23 (s, 3H), 1.50 (d, J=7.0 Hz, 3H).

### Example 27: Preparation of Compound 27

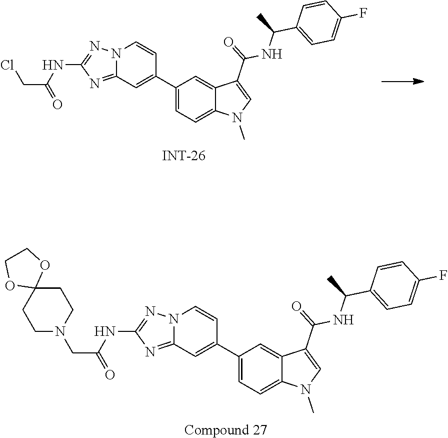 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC6(CC5)OCCO6)nc4c3)cc12)c1ccc(F)cc1 ; "Compound 27"]

### Preparation of Compound 27

Intermediate INT-26 (101 mg, 0.2 mmol) was dissolved in acetonitrile (1 mL); 4-piperidone ethylene ketal (57 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 27 with a yield of 86%. MS (ESI, positive ion) m/z: 612.2 [M+H]⁺.

¹H NMR (400 MHz, DMSO) δ 10.48 (s, 1H), 8.89 (d, J=7.1 Hz, 1H), 8.55 (d, J=1.4 Hz, 1H), 8.39 (d, J=8.0 Hz, 1H), 8.23 (s, 1H), 7.90 (d, J=1.2 Hz, 1H), 7.78-7.64 (m, 2H), 7.52-7.43 (m, 3H), 7.17 (t, J=8.9 Hz, 2H), 5.22 (p, J=7.1 Hz, 1H), 3.90 (d, J=10.6 Hz, 7H), 3.27 (s, 2H), 2.68-2.58 (m, 4H), 1.69 (t, J=5.4 Hz, 4H), 1.50 (d, J=7.0 Hz, 3H).

### Example 28: Preparation of Compound 28

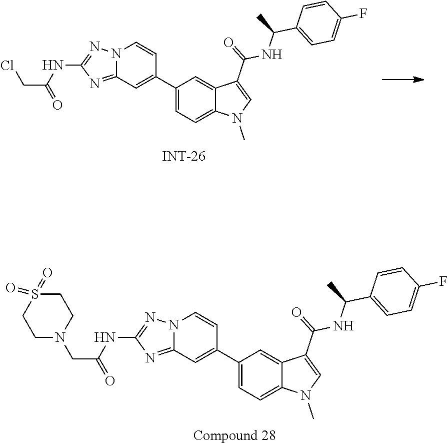 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCS(=O)(=O)CC5)nc4c3)cc12)c1ccc(F)cc1 ; "Compound 28"]

### Preparation of Compound 28

Intermediate INT-26 (101 mg, 0.2 mmol) was dissolved in acetonitrile (1 mL); thiomorpholine 1,1-dioxide (53 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 28 with a yield of 72%. MS (ESI, positive ion) m/z: 604.2 [M+H]⁺.

¹H NMR (400 MHz, DMSO) δ 10.70 (s, 1H), 8.89 (d, J=7.1 Hz, 1H), 8.55 (d, J=1.4 Hz, 1H), 8.39 (d, J=8.0 Hz, 1H), 8.23 (s, 1H), 7.90 (d, J=1.2 Hz, 1H), 7.78-7.59 (m, 2H), 7.52-7.43 (m, 3H), 7.22-7.12 (m, 2H), 5.22 (p, J=7.0 Hz, 1H), 3.91 (s, 3H), 3.52 (s, 2H), 3.19 (d, J=4.8 Hz, 4H), 3.13 (d, J=5.3 Hz, 4H), 1.50 (d, J=7.0 Hz, 3H).

### Example 29: Preparation of Compound 29

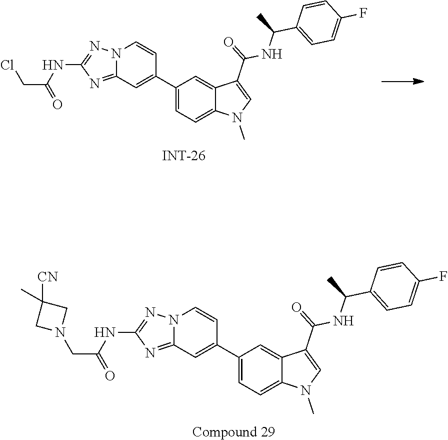 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC(C)(C#N)C5)nc4c3)cc12)c1ccc(F)cc1 ; "Compound 29"]

### Preparation of Compound 29

Intermediate INT-26 (101 mg, 0.2 mmol) was dissolved in acetonitrile (1 mL); 3-methylazetidine-3-carbonitrile (38 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 29 with a yield of 68%. MS (ESI, positive ion) m/z: 565.2 [M+H]⁺.

¹H NMR (400 MHz, DMSO) δ 10.68 (s, 1H), 8.87 (d, J=7.1 Hz, 1H), 8.54 (s, 1H), 8.38 (d, J=8.0 Hz, 1H), 8.22 (s, 1H), 7.89 (s, 1H), 7.77-7.63 (m, 2H), 7.52-7.42 (m, 3H), 7.16 (t, J=8.9 Hz, 2H), 5.21 (p, J=7.2 Hz, 1H), 3.90 (s, 3H), 3.67 (d, J=7.2 Hz, 2H), 3.45 (s, 2H), 3.33 (s, 2H), 1.58 (s, 3H), 1.49 (d, J=7.0 Hz, 3H).

### Example 30: Preparation of Compound 30

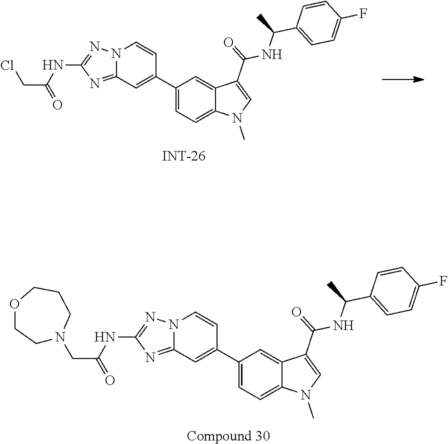 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCCOCC5)nc4c3)cc12)c1ccc(F)cc1 ; "Compound 30"]

### Preparation of Compound 30

Intermediate INT-26 (101 mg, 0.2 mmol) was dissolved in acetonitrile (1 mL); 1,4-oxazepane (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 30 with a yield of 78%. MS (ESI, positive ion) m/z: 570.2 [M+H]⁺.

¹H NMR (400 MHz, DMSO) δ 10.47 (s, 1H), 8.89 (d, J=7.1 Hz, 1H), 8.55 (d, J=1.5 Hz, 1H), 8.39 (d, J=8.0 Hz, 1H), 8.23 (s, 1H), 7.90 (d, J=1.2 Hz, 1H), 7.78-7.64 (m, 2H), 7.52-7.43 (m, 3H), 7.17 (dd, J=12.4, 5.4 Hz, 2H), 5.22 (p, J=7.1 Hz, 1H), 3.91 (s, 3H), 3.73 (t, J=6.0 Hz, 2H), 3.70-3.64 (m, 2H), 3.44 (s, 2H), 2.87-2.80 (m, 4H), 1.92-1.81 (m, 2H), 1.50 (d, J=7.0 Hz, 3H).

### Example 31: Preparation of Compound 31

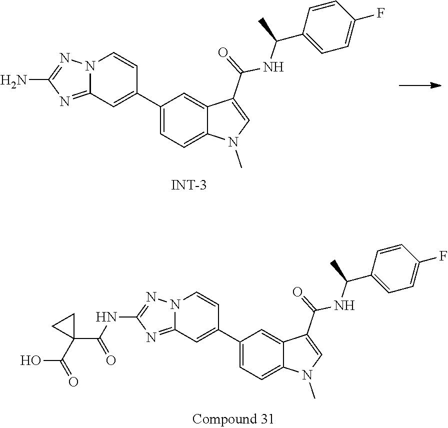

### Preparation of Compound 31

Intermediate INT-3 (85 mg, 0.2 mmol) was dissolved in pyridine (1 mL); 1,1-cyclopropanedicarboxylic acid (39 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 31 with a yield of 39%. MS (ESI, positive ion) m/z: 541.2 [M+H]⁺.

¹H NMR (400 MHz, DMSO-d₆) δ 8.81 (d, J=7.2 Hz, 1H), 8.52 (s, 1H), 8.44 (d, J=7.9 Hz, 1H), 8.28 (s, 1H), 7.86 (s, 1H), 7.64 (q, J=8.8 Hz, 2H), 7.43 (t, J=7.4 Hz, 3H), 7.11 (t, J=8.7 Hz, 2H), 5.24-5.11 (m, 1H), 3.85 (s, 3H), 1.45 (d, J=7.0 Hz, 3H), 1.23 (d, J=41.5 Hz, 4H).

### Example 32: Preparation of Compound 32

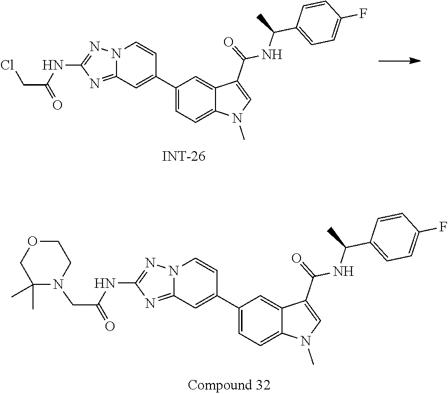 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOCC5(C)C)nc4c3)cc12)c1ccc(F)cc1]

### Preparation of Compound 32

Intermediate INT-26 (101 mg, 0.2 mmol) was dissolved in acetonitrile (1 mL); 3,3-dimethylmorpholine (46 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 32 with a yield of 88%. MS (ESI, positive ion) m/z: 583.2 [M+H]⁺.

¹H NMR (400 MHz, DMSO) δ 10.32-10.25 (m, 1H), 8.94-8.87 (m, 1H), 8.58-8.52 (m, 1H), 8.43-8.34 (m, 1H), 8.26-8.21 (m, 1H), 7.94-7.89 (m, 1H), 7.78-7.64 (m, 2H), 7.53-7.43 (m, 3H), 7.22-7.13 (m, 2H), 5.23 (p, J=7.0 Hz, 1H), 3.92 (s, 3H), 3.76-3.65 (m, 2H), 3.37 (s, 2H), 3.22 (s, 2H), 2.66-2.57 (m, 2H), 1.50 (d, J=7.0 Hz, 3H), 1.04 (s, 6H).

### Example 33: Preparation of Compound 33

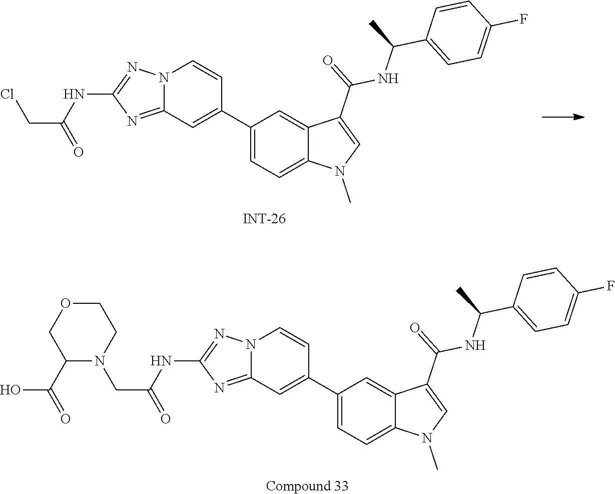 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOCC5C(=O)O)nc4c3)cc12)c1ccc(F)cc1 ; "Compound 33"]

### Preparation of Compound 33

Intermediate INT-26 (108 mg, 0.2 mmol) was dissolved in acetonitrile (1 mL); 3-morpholinecarboxylic acid (52 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 33 with a yield of 48%. MS (ESI, positive ion) m/z: 600.2 [M+H]⁺.

¹H NMR (400 MHz, DMSO-d₆) δ 11.3 (s, 1H), 10.25 (s, 1H), 8.87 (d, J=7.1 Hz, 1H), 8.54 (d, J=1.9 Hz, 1H), 8.39 (d, J=8.0 Hz, 1H), 8.23 (s, 1H), 7.88 (s, 1H), 7.76-7.64 (m, 2H), 7.47 (ddd, J=8.9, 5.7, 2.8 Hz, 3H), 7.20-7.14 (m, 2H), 5.22 (p, J=7.2 Hz, 1H), 3.91 (s, 3H), 3.85 (d, J=9.8 Hz, 1H), 3.72-3.50 (m, 5H), 3.18 (s, 2H), 2.90 (d, J=11.4 Hz, 1H), 1.50 (d, J=7.0 Hz, 3H).

### Example 34: Preparation of Compound 34

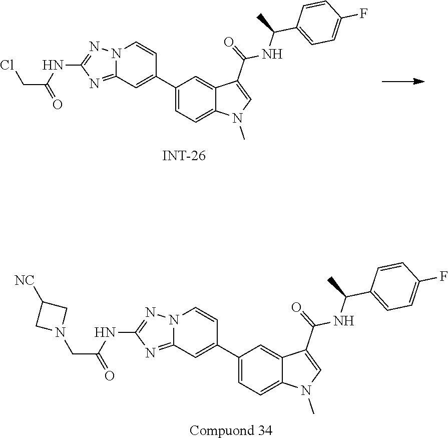 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC(C#N)C5)nc4c3)cc12)c1ccc(F)cc1 ; "Compuond 34"]

### Preparation of Compound 34

Intermediate INT-26 (101 mg, 0.2 mmol) was dissolved in acetonitrile (1 mL); 3-azetidinecarbonitrile (32 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 34 with a yield of 38%. MS (ESI, positive ion) m/z: 551.2 [M+H]⁺.

¹H NMR (400 MHz, DMSO-d₆) δ 10.65 (s, 1H), 8.86 (d, J=7.1 Hz, 1H), 8.54 (d, J=1.8 Hz, 1H), 8.36 (d, J=8.0 Hz, 1H), 8.22 (s, 1H), 7.89 (d, J=1.9 Hz, 1H), 7.72 (d, J=1.9 Hz, 1H), 7.66 (d, J=8.6 Hz, 1H), 7.47 (td, J=5.5, 2.5 Hz, 3H), 7.16 (t, J=8.9 Hz, 2H), 5.22 (t, J=7.3 Hz, 1H), 3.91 (s, 3H), 3.64 (dd, J=7.8, 5.9 Hz, 2H), 3.59-3.53 (m, 1H), 3.48 (d, J=6.3 Hz, 2H), 1.50 (d, J=7.1 Hz, 3H), 0.95 (d, J=6.5 Hz, 2H).

### Example 35: Preparation of Compound 35

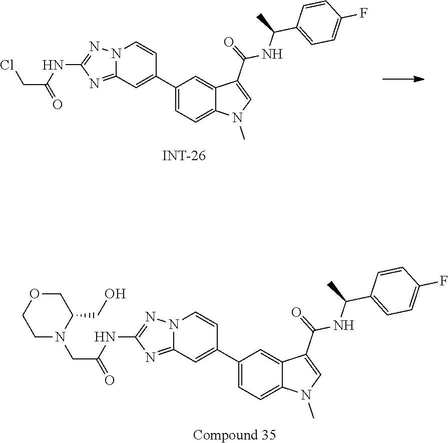 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOC[C@@H]5CO)nc4c3)cc12)c1ccc(F)cc1 ; "Compound 35"]

### Preparation of Compound 35

Intermediate INT-26 (101 mg, 0.2 mmol) was dissolved in acetonitrile (1 mL), and (S)-3-(hydroxymethyl)morpholine (46 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 35 with a yield of 58%. MS (ESI, positive ion) m/z: 586.2 [M+H]⁺.

¹H NMR (400 MHz, DMSO-d₆) δ 10.47 (s, 1H), 8.89 (d, J=7.1 Hz, 1H), 8.59-8.53 (m, 1H), 8.36 (t, J=7.6 Hz, 1H), 8.22 (d, J=7.6 Hz, 1H), 7.90 (d, J=2.0 Hz, 1H), 7.76-7.61 (m, 2H), 7.51-7.43 (m, 3H), 7.17 (t, J=8.8 Hz, 2H), 5.22 (p, J=7.1 Hz, 1H), 4.71 (t, J=5.2 Hz, 1H), 3.91 (d, J=4.8 Hz, 3H), 3.80-3.38 (m, 6H), 3.27 (d, J=16.3 Hz, 2H), 3.06-2.77 (m, 1H), 2.60 (d, J=8.7 Hz, 1H), 2.46 (q, J=7.2 Hz, 1H), 1.50 (dd, J=7.1, 1.9 Hz, 3H).

### Example 36: Preparation of Compound 36

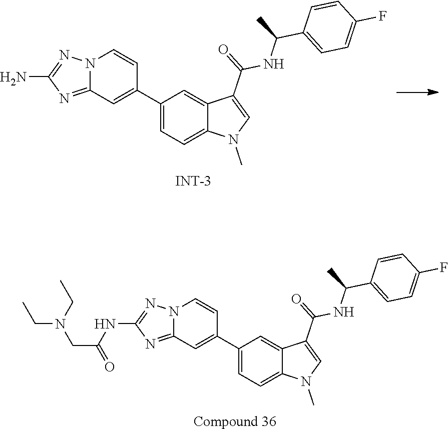 [SMILES: C[C@H](NC(=O)c1cn(C)c2ccc(-c3ccn4nc(N)nc4c3)cc12)c1ccc(F)cc1 ; "INT-3" ; CCN(CC)CC(=O)Nc1nc2cc(-c3ccc4c(c3)c(C(=O)N[C@@H](C)c3ccc(F)cc3)cn4C)ccn2n1 ; "Compound 36"]

### Preparation of Compound 36

Intermediate INT-3 (85 mg, 0.2 mmol) was dissolved in pyridine (1 mL); 2-diethylaminoacetic acid (39 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 36 with a yield of 39%. MS (ESI, positive ion) m/z: 542.2 [M+H]⁺.

¹H NMR (400 MHz, DMSO-d₆) δ 10.28 (s, 1H), 8.89 (d, J=7.1 Hz, 1H), 8.55 (d, J=1.8 Hz, 1H), 8.37 (d, J=8.0 Hz, 1H), 8.23 (s, 1H), 7.90 (d, J=1.9 Hz, 1H), 7.73 (d, J=1.9 Hz, 1H), 7.67 (d, J=8.6 Hz, 1H), 7.50-7.46 (m, 3H), 7.17 (t, J=8.9 Hz, 2H), 5.23 (t, J=7.4 Hz, 1H), 3.91 (s, 3H), 3.28 (s, 2H), 2.65 (q, J=7.2 Hz, 4H), 1.51 (d, J=7.1 Hz, 3H), 1.05 (t, J=7.1 Hz, 6H).

### Example 37: Preparation of Compound 37

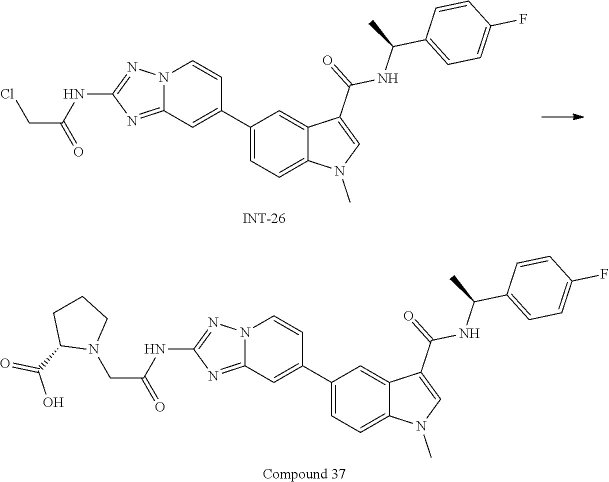 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC[C@H]5C(=O)O)nc4c3)cc12)c1ccc(F)cc1 ; "Compound 37"]

### Preparation of Compound 37

Intermediate INT-26 (101 mg, 0.2 mmol) was dissolved in acetonitrile (1 mL); L-proline (34 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 37 with a yield of 48%. MS (ESI, positive ion) m/z: 584.2 [M+H]⁺.

¹H NMR (400 MHz, DMSO-d₆) δ 8.87 (d, J=7.2 Hz, 1H), 8.55 (s, 1H), 8.37 (d, J=7.9 Hz, 1H), 8.22 (s, 1H), 7.89 (s, 1H), 7.78-7.64 (m, 2H), 7.47 (dd, J=8.6, 5.6 Hz, 3H), 7.17 (t, J=8.8 Hz, 2H), 5.22 (t, J=7.3 Hz, 1H), 3.91 (s, 3H), 3.50-3.42 (m, 2H), 3.28-3.09 (m, 4H), 2.67 (d, J=15.5 Hz, 1H), 2.19-2.06 (m, 1H), 1.50 (d, J=7.1 Hz, 3H).

### Example 38: Preparation of Compound 38

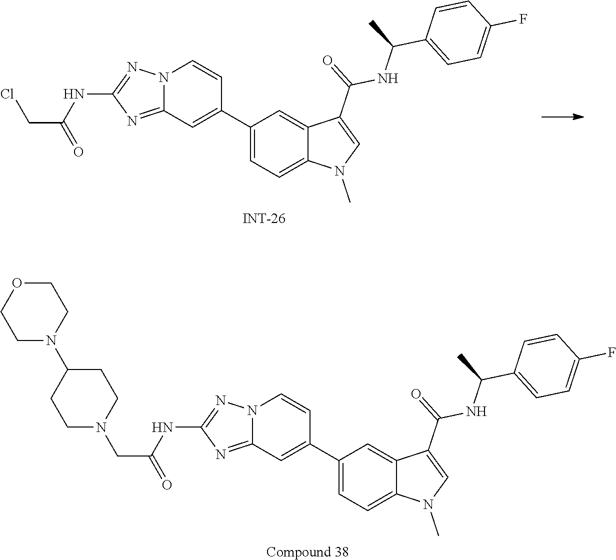 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC(N6CCOCC6)CC5)nc4c3)cc12)c1ccc(F)cc1 ; "Compound 38"]

### Preparation of Compound 38

Intermediate INT-26 (101 mg, 0.2 mmol) was dissolved in acetonitrile (1 mL); 4-(4-piperidinyl)morpholine (68 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 38 with a yield of 78%. MS (ESI, positive ion) m/z: 639.2 [M+H]⁺.

¹H NMR (400 MHz, DMSO-d₆) δ 10.40 (s, 1H), 8.88 (dd, J=7.1, 0.8 Hz, 1H), 8.55 (d, J=1.8 Hz, 1H), 8.36 (d, J=8.0 Hz, 1H), 8.22 (s, 1H), 7.90 (dd, J=2.0, 0.9 Hz, 1H), 7.74 (dd, J=8.7, 1.9 Hz, 1H), 7.67 (d, J=8.6 Hz, 1H), 7.50-7.45 (m, 3H), 7.21-7.13 (m, 2H), 5.23 (t, J=7.4 Hz, 1H), 3.91 (s, 3H), 3.58 (t, J=4.6 Hz, 4H), 3.22 (s, 2H), 2.95 (d, J=11.0 Hz, 2H), 2.47 (t, J=4.7 Hz, 4H), 2.24-2.16 (m, 2H), 1.77 (d, J=12.2 Hz, 2H), 1.49 (t, J=8.3 Hz, 5H).

### Example 39: Preparation of Compound 39

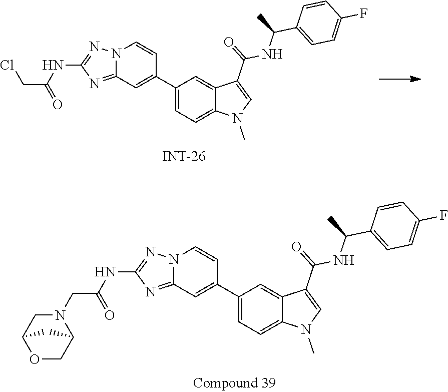 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5C[C@H]6C[C@@H]5CO6)nc4c3)cc12)c1ccc(F)cc1]

### Preparation of Compound 39

Intermediate INT-26 (101 mg, 0.2 mmol) was dissolved in acetonitrile (1 mL), and (1R,4R)-2-oxa-5-azabicyclo[2.2.1]heptane (39 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 39 with a yield of 76%. MS (ESI, positive ion) m/z: 568.2 [M+H]⁺.

¹H NMR (400 MHz, DMSO-d₆) δ 10.39 (s, 1H), 8.89 (d, J=7.1 Hz, 1H), 8.55 (d, J=1.8 Hz, 1H), 8.37 (d, J=8.0 Hz, 1H), 8.23 (s, 1H), 7.90 (d, J=1.9 Hz, 1H), 7.77-7.71 (m, 1H), 7.67 (d, J=8.6 Hz, 1H), 7.50-7.45 (m, 3H), 7.17 (t, J=8.7 Hz, 2H), 5.23 (t, J=7.3 Hz, 1H), 4.40 (s, 1H), 3.90 (d, J=10.6 Hz, 4H), 3.62 (s, 1H), 3.58 (dd, J=7.6, 1.7 Hz, 1H), 3.48 (d, J=5.3 Hz, 2H), 2.97 (dd, J=10.0, 1.7 Hz, 1H), 2.57 (d, J=9.9 Hz, 1H), 1.87 (dd, J=9.6, 2.1 Hz, 1H), 1.65 (d, J=9.5 Hz, 1H), 1.51 (d, J=7.0 Hz, 3H).

### Example 40: Preparation of Compound 40

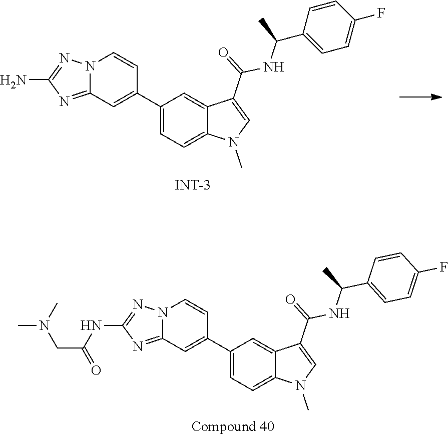 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)CN(C)C)nc4c3)cc12)c1ccc(F)cc1 ; "Compound 40"]

### Preparation of Compound 40

Intermediate INT-3 (85 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 40 with a yield of 39%. MS (ESI, positive ion) m/z: 514.2 [M+H]⁺.

¹H NMR (400 MHz, DMSO-d₆) δ 10.76 (s, 1H), 8.87 (d, J=7.1 Hz, 1H), 8.52 (d, J=1.8 Hz, 1H), 8.37 (d, J=8.0 Hz, 1H), 8.23 (s, 1H), 7.89 (d, J=1.9 Hz, 1H), 7.76 (dd, J=8.6, 1.9 Hz, 1H), 7.65 (d, J=8.6 Hz, 1H), 7.57 (td, J=5.3, 2.4 Hz, 3H), 7.19-7.14 (m, 2H), 5.22 (p, J=7.2 Hz, 1H), 3.91 (s, 3H), 3.80 (t, J=12.5 Hz, 4H), 3.61 (s, 6H), 1.50 (d, J=7.0 Hz, 3H), 0.97 (d, J=6.5 Hz, 2H).

### Example 41: Preparation of Compound 41

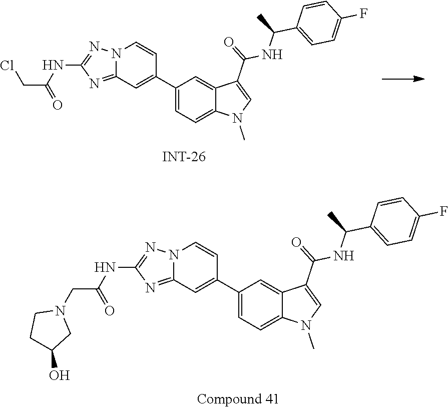 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@H](O)C5)nc4c3)cc12)c1ccc(F)cc1]

### Preparation of Compound 41

Intermediate INT-26 (101 mg, 0.2 mmol) was dissolved in acetonitrile (1 mL); (S)-3-pyrrolidinol (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 41 with a yield of 66%. MS (ESI, positive ion) m/z: 556.2 [M+H]⁺.

¹H NMR (400 MHz, DMSO-d₆) δ 10.74 (s, 1H), 8.89 (d, J=7.1 Hz, 1H), 8.54 (d, J=1.8 Hz, 1H), 8.36 (d, J=8.0 Hz, 1H), 8.22 (s, 1H), 7.89 (d, J=1.9 Hz, 1H), 7.74 (dd, J=8.6, 1.9 Hz, 1H), 7.67 (d, J=8.6 Hz, 1H), 7.47 (td, J=5.3, 2.4 Hz, 3H), 7.19-7.14 (m, 2H), 5.22 (p, J=7.2 Hz, 1H), 3.91 (s, 3H), 3.80 (t, J=12.5 Hz, 4H), 2.06 (m, 1H), 1.50 (d, J=7.0 Hz, 3H), 1.22 (m, 2H), 0.97 (d, J=6.5 Hz, 2H).

### Example 42: Preparation of Compound 42

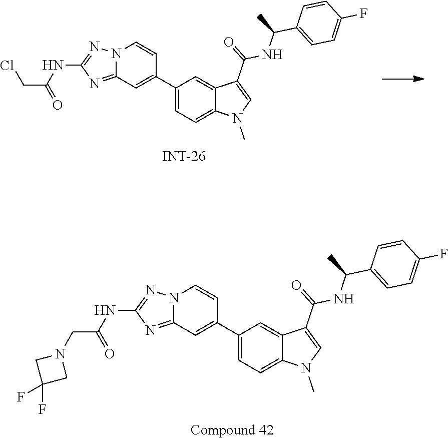 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC(F)(F)C5)nc4c3)cc12)c1ccc(F)cc1 ; "Compound 42"]

### Preparation of Compound 42

Intermediate INT-26 (101 mg, 0.2 mmol) was dissolved in acetonitrile (1 mL); 3,3-difluoroazetidine (37 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 42 with a yield of 71%. MS (ESI, positive ion) m/z: 562.2 [M+H]⁺.

¹H NMR (400 MHz, DMSO-d₆) δ 10.74 (s, 1H), 8.87 (d, J=7.1 Hz, 1H), 8.55 (d, J=1.8 Hz, 1H), 8.37 (d, J=8.0 Hz, 1H), 8.23 (s, 1H), 7.89 (d, J=1.9 Hz, 1H), 7.74 (dd, J=8.6, 1.9 Hz, 1H), 7.67 (d, J=8.6 Hz, 1H), 7.47 (td, J=5.3, 2.4 Hz, 3H), 7.19-7.14 (m, 2H), 5.22 (p, J=7.2 Hz, 1H), 3.91 (s, 3H), 3.80 (t, J=12.5 Hz, 4H), 1.50 (d, J=7.0 Hz, 3H), 0.97 (d, J=6.5 Hz, 2H).

### Example 43: Preparation of Compound 43

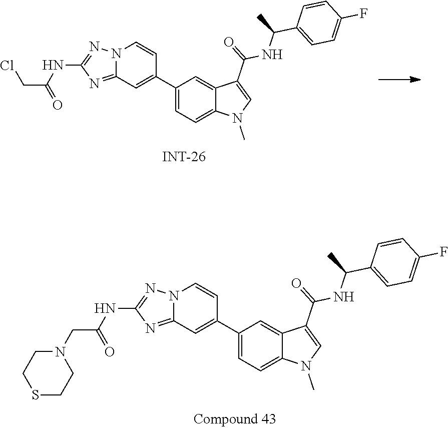 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCSCC5)nc4c3)cc12)c1ccc(F)cc1]

### Preparation of Compound 43

Intermediate INT-26 (101 mg, 0.2 mmol) was dissolved in acetonitrile (1 mL); thiomorpholine (41 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 43 with a yield of 71%. MS (ESI, positive ion) m/z: 562.2 [M+H]⁺.

¹H NMR (400 MHz, DMSO-d₆) δ 10.47 (s, 1H), 8.89 (d, J=7.1 Hz, 1H), 8.56 (d, J=1.9 Hz, 1H), 8.37 (d, J=8.0 Hz, 1H), 8.23 (s, 1H), 7.90 (d, J=1.9 Hz, 1H), 7.74 (dd, J=8.6, 1.9 Hz, 1H), 7.67 (d, J=8.6 Hz, 1H), 7.48 (td, J=5.5, 2.5 Hz, 3H), 7.22-7.12 (m, 2H), 5.23 (p, J=7.1 Hz, 1H), 3.91 (s, 3H), 2.84 (dd, J=6.5, 3.4 Hz, 4H), 2.71-2.65 (m, 4H), 1.51 (d, J=7.0 Hz, 3H).

### Example 44: Preparation of Compound 44

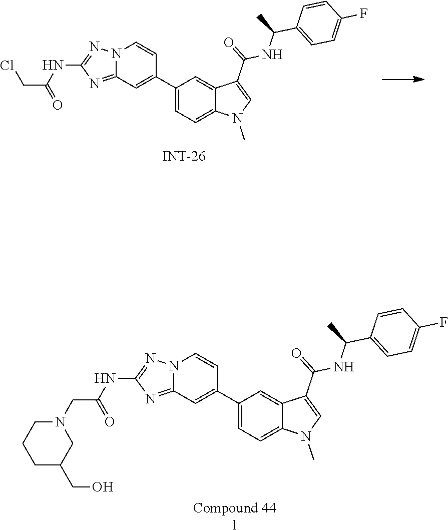 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCCC(CO)C5)nc4c3)cc12)c1ccc(F)cc1 ; "Compound 441"]

### Preparation of Compound 44

Intermediate INT-26 (101 mg, 0.2 mmol) was dissolved in acetonitrile (1 mL); 3-(hydroxymethyl)piperidine (46 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 44 with a yield of 61%. MS (ESI, positive ion) m/z: 584.2 [M+H]⁺.

¹H NMR (400 MHz, DMSO-d₆) δ 10.37 (s, 1H), 8.89 (d, J=7.3 Hz, 1H), 8.55 (d, J=2.0 Hz, 1H), 8.38 (d, J=8.0 Hz, 1H), 8.23 (s, 1H), 7.90 (d, J=1.9 Hz, 1H), 7.74 (dd, J=8.7, 1.9 Hz, 1H), 7.68 (d, J=8.6 Hz, 1H), 7.52-7.43 (m, 3H), 7.22-7.12 (m, 2H), 5.22 (p, J=7.2 Hz, 1H), 4.48 (t, J=5.3 Hz, 1H), 3.92 (s, 3H), 3.34-3.29 (m, 1H), 3.27-3.16 (m, 3H), 2.98-2.91 (m, 1H), 2.83 (d, J=11.0 Hz, 1H), 2.21-2.11 (m, 1H), 1.91 (t, J=10.4 Hz, 1H), 1.75-1.62 (m, 3H), 1.50 (d, J=7.1 Hz, 4H), 0.95 (dd, J=19.6, 9.2 Hz, 1H).

### Example 45: Preparation of Compound 45

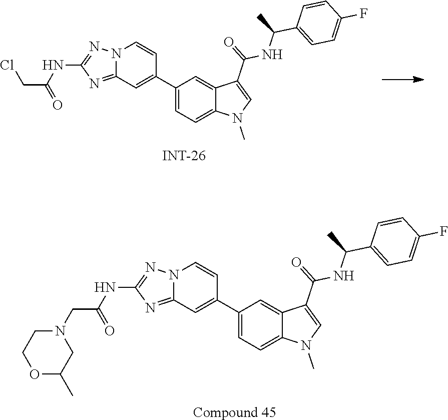 [SMILES: C[C@H](NC(=O)c1cn(C)c2ccc(-c3ccn4nc(NC(=O)CCl)nc4c3)cc12)c1ccc(F)cc1 ; "INT-26" ; CC1CN(CC(=O)Nc2nc3cc(-c4ccc5c(c4)c(C(=O)N[C@@H](C)c4ccc(F)cc4)cn5C)ccn3n2)CCO1 ; "Compound 45"]

### Preparation of Compound 45

Intermediate INT-26 (101 mg, 0.2 mmol) was dissolved in acetonitrile (1 mL); 3-(hydroxymethyl)piperidine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 45 with a yield of 61%. MS (ESI, positive ion) m/z: 570.2 [M+H]⁺.

¹H NMR (400 MHz, DMSO-d₆) δ 10.53 (s, 1H), 8.89 (d, J=7.1 Hz, 1H), 8.55 (d, J=1.8 Hz, 1H), 8.39 (d, J=8.0 Hz, 1H), 8.23 (s, 1H), 7.90 (d, J=1.9 Hz, 1H), 7.74 (dd, J=8.7, 1.9 Hz, 1H), 7.67 (d, J=8.6 Hz, 1H), 7.49-7.45 (m, 3H), 7.17 (t, J=8.9 Hz, 2H), 5.22 (p, J=7.2 Hz, 1H), 3.91 (s, 3H), 3.77 (dt, J=10.4, 2.5 Hz, 1H), 3.64-3.54 (m, 2H), 3.26 (s, 2H), 2.82 (dd, J=11.1, 2.4 Hz, 1H), 2.75 (d, J=11.2 Hz, 1H), 2.28 (td, J=11.3, 3.3 Hz, 1H), 1.98 (t, J=10.5 Hz, 1H), 1.50 (d, J=7.0 Hz, 3H), 1.07 (d, J=6.3 Hz, 3H).

### Example 46: Preparation of Compound 46

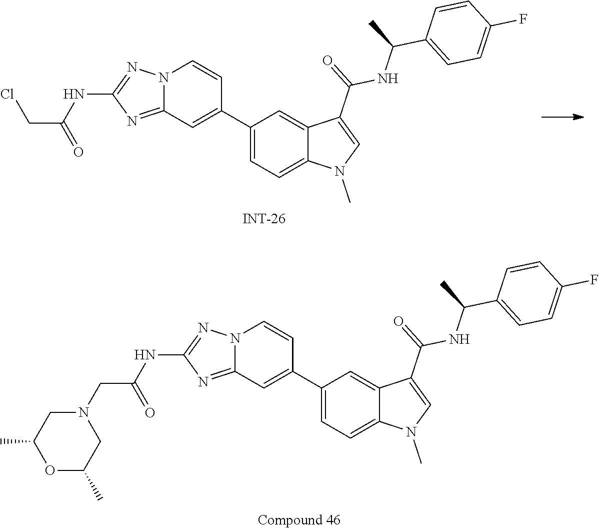 [SMILES: C[C@H](NC(=O)c1cn(C)c2ccc(-c3ccn4nc(NC(=O)CCl)nc4c3)cc12)c1ccc(F)cc1 ; "INT-26" ; C[C@@H]1CN(CC(=O)Nc2nc3cc(-c4ccc5c(c4)c(C(=O)N[C@@H](C)c4ccc(F)cc4)cn5C)ccn3n2)C[C@H](C)O1]

### Preparation of Compound 46

Intermediate INT-26 (101 mg, 0.2 mmol) was dissolved in acetonitrile (1 mL), and cis-2,6-dimethylmorpholine (46 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 46 with a yield of 74%. MS (ESI, positive ion) m/z: 584.2 [M+H]⁺.

¹H NMR (400 MHz, DMSO-d₆) δ 10.49 (s, 1H), 8.88 (dd, J=7.1, 0.8 Hz, 1H), 8.55 (d, J=1.8 Hz, 1H), 8.36 (d, J=8.0 Hz, 1H), 8.22 (s, 1H), 7.89 (dd, J=1.9, 0.9 Hz, 1H), 7.73 (dd, J=8.7, 1.9 Hz, 1H), 7.67 (d, J=8.6 Hz, 1H), 7.49-7.44 (m, 3H), 7.21-7.12 (m, 2H), 5.22 (t, J=7.3 Hz, 1H), 3.91 (s, 3H), 3.64 (ddd, J=10.0, 6.2, 2.0 Hz, 2H), 3.25 (s, 2H), 2.82-2.77 (m, 2H), 1.91 (t, J=10.6 Hz, 2H), 1.50 (d, J=7.0 Hz, 3H), 1.07 (d, J=6.2 Hz, 6H).

### Example 47: Preparation of Compound 47

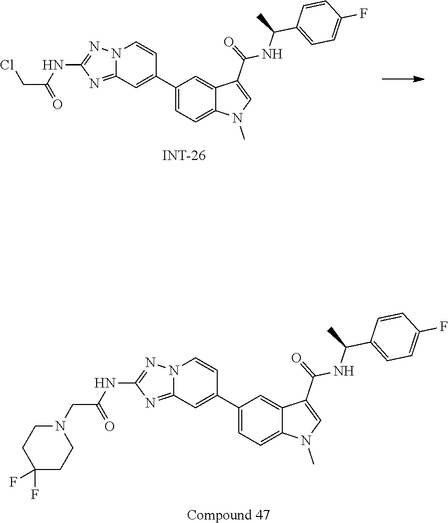 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC(F)(F)CC5)nc4c3)cc12)c1ccc(F)cc1 ; "Compound 47"]

### Preparation of Compound 47

Intermediate INT-26 (101 mg, 0.2 mmol) was dissolved in acetonitrile (1 mL); 4,4-difluoropiperidine (48 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 47 with a yield of 74%. MS (ESI, positive ion) m/z: 590.2 [M+H]⁺.

¹H NMR (400 MHz, DMSO-d₆) δ 10.60 (s, 1H), 8.89 (d, J=7.1 Hz, 1H), 8.55 (d, J=1.9 Hz, 1H), 8.39 (d, J=8.0 Hz, 1H), 8.24 (s, 1H), 7.90 (d, J=2.0 Hz, 1H), 7.74 (dd, J=8.7, 1.9 Hz, 1H), 7.67 (d, J=8.6 Hz, 1H), 7.50-7.45 (m, 3H), 7.22-7.12 (m, 2H), 5.22 (p, J=7.2 Hz, 1H), 3.91 (s, 3H), 2.72 (t, J=5.7 Hz, 4H), 2.53 (d, J=1.9 Hz, 2H), 2.04 (dq, J=14.3, 8.0, 6.8 Hz, 4H), 1.50 (d, J=7.0 Hz, 3H).

### Example 48: Preparation of Compound 48

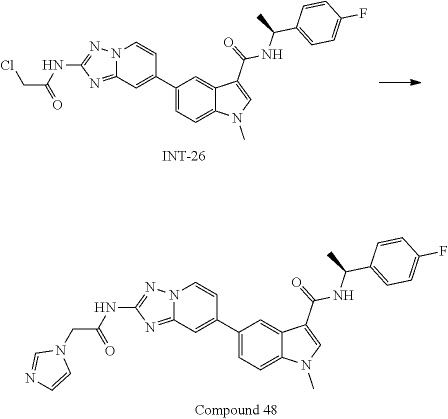 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)Cn5ccnc5)nc4c3)cc12)c1ccc(F)cc1]

### Preparation of Compound 48

Intermediate INT-26 (101 mg, 0.2 mmol) was dissolved in acetonitrile (1 mL); 4,4-difluoropiperidine (27 mg, 0.4 mmol) and potassium carbonate (55 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 48 with a yield of 69%. MS (ESI, positive ion) m/z: 537.2 [M+H]⁺.

¹H NMR (400 MHz, DMSO-d₆) δ 11.25 (s, 1H), 8.90 (d, J=7.1 Hz, 1H), 8.56 (d, J=1.9 Hz, 1H), 8.38 (d, J=8.0 Hz, 1H), 8.23 (s, 1H), 7.93 (t, J=1.4 Hz, 1H), 7.76 (dd, J=8.7, 1.9 Hz, 1H), 7.71-7.64 (m, 2H), 7.47 (tt, J=9.0, 2.6 Hz, 3H), 7.22-7.12 (m, 3H), 6.92 (t, J=1.1 Hz, 1H), 5.23 (q, J=7.3 Hz, 1H), 3.92 (s, 3H), 2.56 (s, 2H), 1.50 (d, J=7.0 Hz, 3H).

### Example 49: Preparation of Compound 49

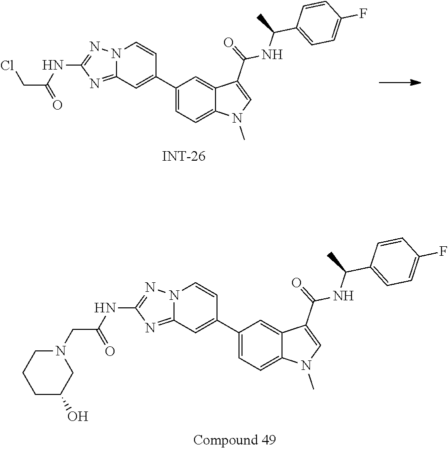 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCC[C@@H](O)C5)nc4c3)cc12)c1ccc(F)cc1]

### Preparation of Compound 49

Intermediate INT-26 (101 mg, 0.2 mmol) was dissolved in acetonitrile (1 mL); (R)-3-piperidinol (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 49 with a yield of 62%. MS (ESI, positive ion) m/z: 570.2 [M+H]⁺.

¹H NMR (400 MHz, DMSO-d₆) δ 10.45 (s, 1H), 8.89 (d, J=7.1 Hz, 1H), 8.55 (d, J=1.8 Hz, 1H), 8.38 (d, J=8.0 Hz, 1H), 8.23 (s, 1H), 7.90 (d, J=1.9 Hz, 1H), 7.74 (dd, J=8.7, 1.9 Hz, 1H), 7.68 (d, J=8.6 Hz, 1H), 7.48 (dt, J=6.6, 2.7 Hz, 3H), 7.17 (dd, J=10.0, 7.8 Hz, 2H), 5.23 (q, J=7.3 Hz, 1H), 4.77 (d, J=5.6 Hz, 1H), 3.91 (s, 3H), 3.58 (s, 1H), 3.30-3.15 (m, 2H), 2.83 (d, J=10.0 Hz, 1H), 2.64 (d, J=10.9 Hz, 1H), 2.17 (dt, J=42.9, 9.8 Hz, 2H), 1.74 (q, J=12.9, 9.1 Hz, 2H), 1.50 (d, J=7.0 Hz, 4H), 1.24-1.12 (m, 1H).

### Example 50: Preparation of Compound 50

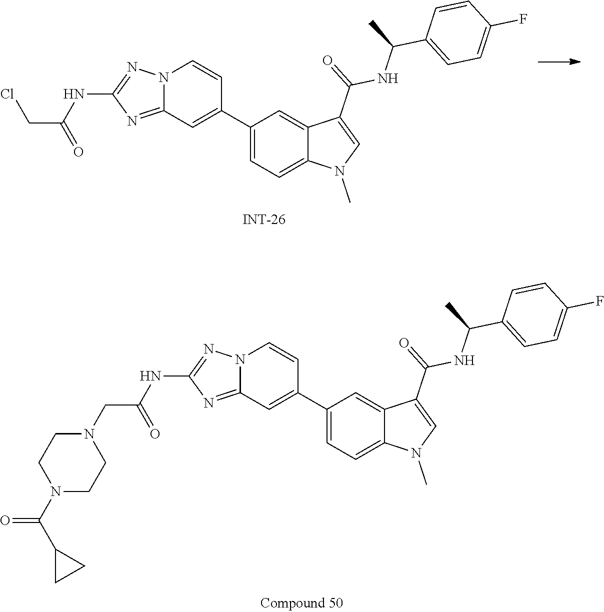 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCN(C(=O)C6CC6)CC5)nc4c3)cc12)c1ccc(F)cc1]

### Preparation of Compound 50

Intermediate INT-26 (101 mg, 0.2 mmol) was dissolved in acetonitrile (1 mL); 1-cyclopropanecarbonylpiperazine (61 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 50 with a yield of 58%. MS (ESI, positive ion) m/z: 623.2 [M+H]⁺.

¹H NMR (400 MHz, DMSO-d₆) δ 10.58 (s, 1H), 8.89 (d, J=7.1 Hz, 1H), 8.55 (d, J=1.9 Hz, 1H), 8.38 (d, J=8.0 Hz, 1H), 8.23 (s, 1H), 7.91 (d, J=2.0 Hz, 1H), 7.74 (dd, J=8.7, 1.9 Hz, 1H), 7.67 (d, J=8.7 Hz, 1H), 7.50-7.45 (m, 3H), 7.20-7.14 (m, 2H), 5.22 (p, J=7.2 Hz, 1H), 3.91 (s, 3H), 3.74 (s, 2H), 3.53 (s, 2H), 3.34 (s, 4H), 2.62 (s, 2H), 1.99 (tt, J=7.8, 4.9 Hz, 1H), 1.50 (d, J=7.0 Hz, 3H), 0.73 (ddt, J=10.7, 4.8, 2.9 Hz, 4H).

### Example 51: Preparation of Compound 51

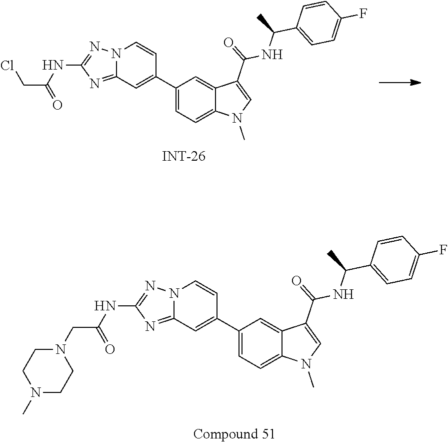 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCN(C)CC5)nc4c3)cc12)c1ccc(F)cc1]

### Preparation of Compound 51

Intermediate INT-26 (101 mg, 0.2 mmol) was dissolved in acetonitrile (1 mL); 1-methylpiperazine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 51 with a yield of 61%. MS (ESI, positive ion) m/z: 569.2 [M+H]⁺.

¹H NMR (400 MHz, DMSO-d₆) δ 10.44 (s, 1H), 8.89 (d, J=7.1 Hz, 1H), 8.55 (d, J=1.8 Hz, 1H), 8.39 (d, J=8.0 Hz, 1H), 8.23 (s, 1H), 7.90 (d, J=2.0 Hz, 1H), 7.74 (dd, J=8.6, 1.9 Hz, 1H), 7.67 (d, J=8.6 Hz, 1H), 7.48 (td, J=5.6, 2.6 Hz, 3H), 7.17 (t, J=8.9 Hz, 2H), 5.23 (p, J=7.1 Hz, 1H), 3.92 (s, 3H), 3.36 (s, 6H), 2.79-2.54 (m, 4H), 2.38 (s, 3H), 1.50 (d, J=7.1 Hz, 3H).

### Example 52: Preparation of Compound 52

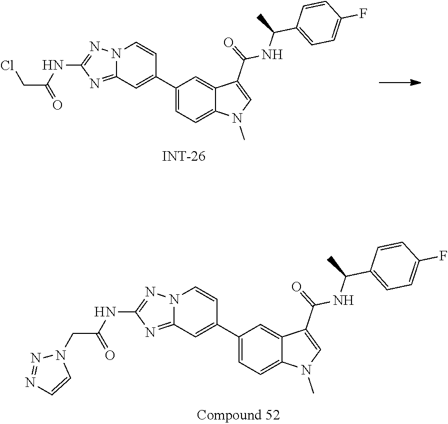 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)Cn5ccnn5)nc4c3)cc12)c1ccc(F)cc1]

### Preparation of Compound 52

Intermediate INT-26 (101 mg, 0.2 mmol) was dissolved in acetonitrile (1 mL); 1,2,3-triazole (27 mg, 0.4 mmol) and potassium carbonate (55 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 52 with a yield of 41%. MS (ESI, positive ion) m/z: 538.2 [M+H]⁺.

¹H NMR (400 MHz, DMSO-d₆) δ 10.54 (s, 1H), 8.91 (d, J=7.2 Hz, 1H), 8.56 (d, J=1.9 Hz, 1H), 8.38 (d, J=8.0 Hz, 1H), 8.23 (s, 1H), 8.19 (s, 1H), 7.97-7.93 (m, 1H), 7.79 (d, J=9.1 Hz, 1H), 7.68 (d, J=8.6 Hz, 1H), 7.47 (dd, J=8.5, 5.7 Hz, 3H), 7.17 (dd, J=10.1, 7.7 Hz, 2H), 5.22 (t, J=7.3 Hz, 1H), 3.92 (s, 3H), 2.56 (s, 2H), 1.50 (d, J=7.0 Hz, 3H), 0.97 (d, J=6.5 Hz, 1H).

### Example 53: Preparation of Compound 53

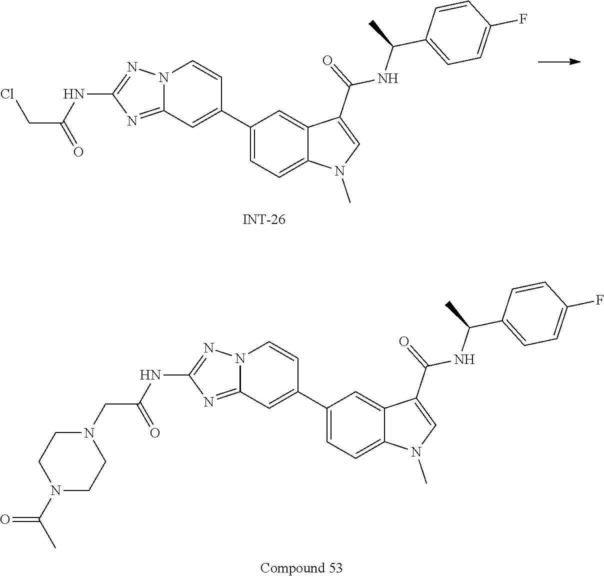 [SMILES: C[C@H](NC(=O)c1cn(C)c2ccc(-c3ccn4nc(NC(=O)CCl)nc4c3)cc12)c1ccc(F)cc1 ; "INT-26" ; CC(=O)N1CCN(CC(=O)Nc2nc3cc(-c4ccc5c(c4)c(C(=O)N[C@@H](C)c4ccc(F)cc4)cn5C)ccn3n2)CC1]

### Preparation of Compound 53

Intermediate INT-26 (101 mg, 0.2 mmol) was dissolved in acetonitrile (1 mL); 1-acetylpiperazine (51 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 53 with a yield of 58%. MS (ESI, positive ion) m/z: 597.2 [M+H]⁺.

¹H NMR (400 MHz, DMSO-d₆) δ 10.54 (s, 1H), 8.89 (d, J=7.1 Hz, 1H), 8.55 (d, J=1.8 Hz, 1H), 8.39 (d, J=8.0 Hz, 1H), 8.23 (s, 1H), 7.91 (d, J=2.0 Hz, 1H), 7.74 (dd, J=8.7, 1.9 Hz, 1H), 7.68 (d, J=8.6 Hz, 1H), 7.50-7.45 (m, 3H), 7.20-7.15 (m, 2H), 5.22 (p, J=7.2 Hz, 1H), 3.92 (s, 3H), 3.50 (q, J=5.1 Hz, 4H), 3.32 (s, 4H), 2.58 (t, J=5.0 Hz, 2H), 2.02 (s, 3H), 1.50 (d, J=7.0 Hz, 3H).

### Example 54: Preparation of Compound 54

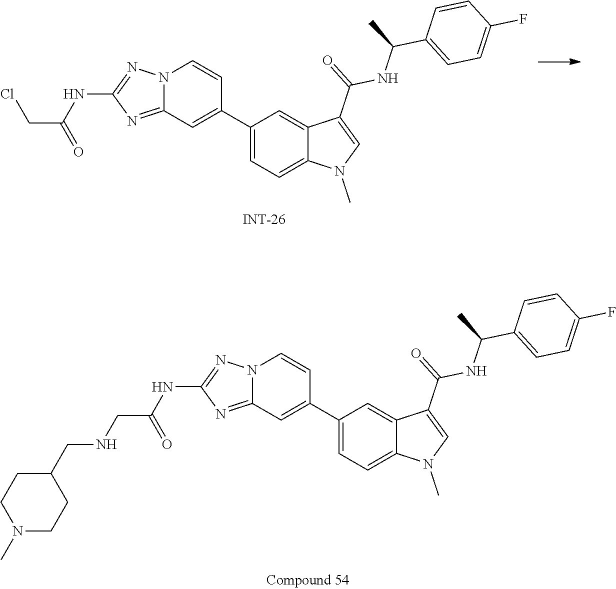 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNCC5CCN(C)CC5)nc4c3)cc12)c1ccc(F)cc1]

### Preparation of Compound 54

Intermediate INT-26 (101 mg, 0.2 mmol) was dissolved in acetonitrile (1 mL); (1-methylpiperidin-4-yl)methanamine (51 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 54 with a yield of 54%. MS (ESI, positive ion) m/z: 597.2 [M+H]⁺.

¹H NMR (400 MHz, DMSO-d₆) δ 10.53 (s, 1H), 8.88 (d, J=7.1 Hz, 1H), 8.55 (d, J=1.9 Hz, 1H), 8.38 (d, J=8.1 Hz, 1H), 8.22 (s, 1H), 7.90 (d, J=1.9 Hz, 1H), 7.76 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 5.22 (p, J=7.2 Hz, 1H), 4.32 (m, 1H), 3.91 (s, 3H), 3.64 (t, J=4.6 Hz, 4H), 3.5 (s, 3H), 3.27 (s, 2H), 3.10 (m, 2H) 1.50 (d, J=7.0 Hz, 3H), 1.23 (m, 4H), 1.15 (m, 1H).

### Example 55: Preparation of Compound 55

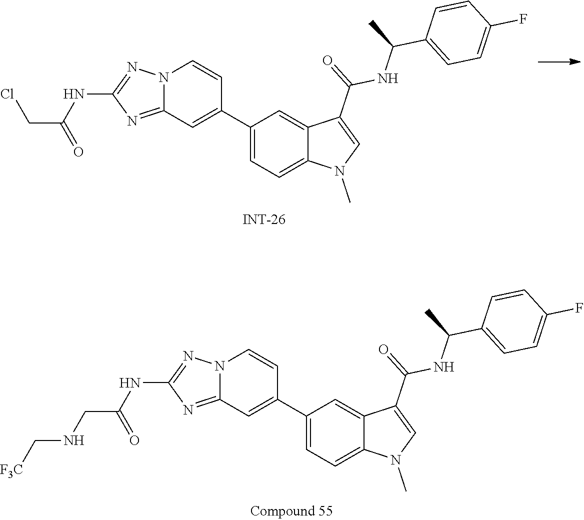 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNCC(F)(F)F)nc4c3)cc12)c1ccc(F)cc1]

### Preparation of Compound 55

Intermediate INT-26 (101 mg, 0.2 mmol) was dissolved in acetonitrile (1 mL); trifluoroethylamine (39 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 55 with a yield of 54%. MS (ESI, positive ion) m/z: 568.2 [M+H]⁺. 30 ¹H NMR (400 MHz, DMSO-d₆) δ 10.74 (s, 1H), 8.91-8.87 (m, 1H), 8.55 (d, J=1.9 Hz, 1H), 8.38 (d, J=8.0 Hz, 1H), 8.23 (s, 1H), 7.92-7.89 (m, 1H), 7.75 (dd, J=8.7, 1.9 Hz, 1H), 7.67 (d, J=8.7 Hz, 1H), 7.47 (td, J=5.5, 2.5 Hz, 3H), 7.19-7.15 (m, 2H), 5.22 (t, J=7.3 Hz, 1H), 3.91 (s, 3H), 3.61 (s, 1H), 3.40 (td, J=10.1, 6.5 Hz, 2H), 1.50 (d, J=7.1 Hz, 3H), 0.97 (d, J=6.5 Hz, 2H).

### Example 56: Preparation of Compound 56

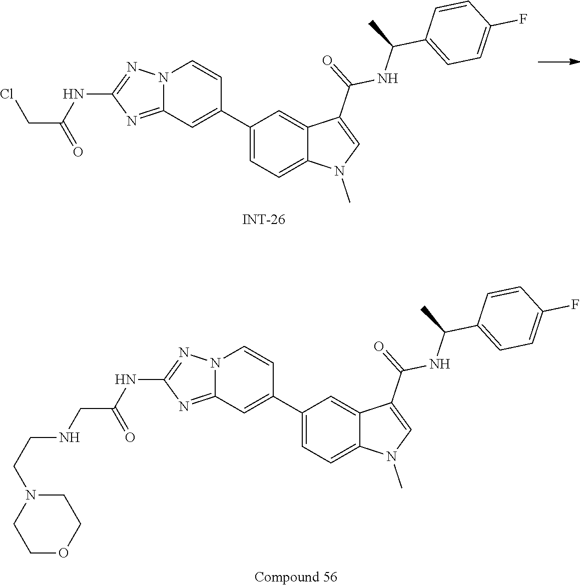 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNCCN5CCOCC5)nc4c3)cc12)c1ccc(F)cc1]

### Preparation of Compound 56

Intermediate INT-26 (101 mg, 0.2 mmol) was dissolved in acetonitrile (1 mL); N-(2-aminoethyl)morpholine (52 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 56 with a yield of 64%. MS (ESI, positive ion) m/z: 599.2 [M+H]⁺.

¹H NMR (400 MHz, DMSO-d₆) δ 8.89 (d, J=7.1 Hz, 1H), 8.56 (d, J=1.8 Hz, 1H), 8.37 (d, J=8.0 Hz, 1H), 8.23 (s, 1H), 7.90 (d, J=2.0 Hz, 1H), 7.75 (dd, J=8.6, 1.9 Hz, 1H), 7.67 (d, J=8.6 Hz, 1H), 7.48 (td, J=5.4, 2.4 Hz, 3H), 7.17 (t, J=8.8 Hz, 2H), 5.23 (p, J=7.2 Hz, 1H), 3.92 (s, 3H), 3.60 (t, J=4.7 Hz, 4H), 3.47 (s, 2H), 2.70 (t, J=6.1 Hz, 2H), 2.41 (dt, J=10.4, 5.4 Hz, 6H), 1.51 (d, J=7.0 Hz, 3H).

### Example 57: Preparation of Compound 57

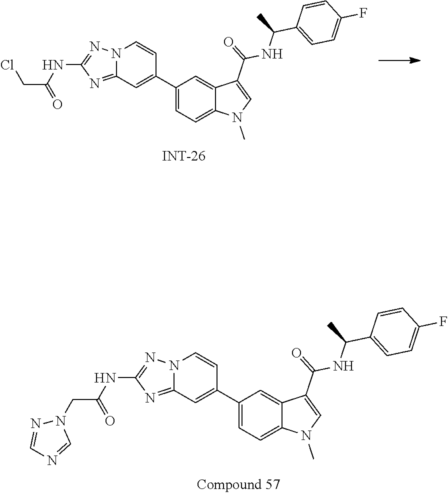 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)Cn5cncn5)nc4c3)cc12)c1ccc(F)cc1]

### Preparation of Compound 57

Intermediate INT-26 (101 mg, 0.2 mmol) was dissolved in acetonitrile (1 mL); 1,2,4-triazole (27 mg, 0.4 mmol) and potassium carbonate (55 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 57 with a yield of 41%. MS (ESI, positive ion) m/z: 538.2 [M+H]⁺.

¹H NMR (400 MHz, DMSO-d₆) δ 8.89 (d, J=7.2 Hz, 1H), 8.55 (d, J=1.9 Hz, 1H), 8.52 (s, 1H), 8.40 (d, J=8.0 Hz, 1H), 8.22 (s, 1H), 7.93 (d, J=1.9 Hz, 1H), 7.75 (dd, J=8.7, 1.9 Hz, 1H), 7.67 (d, J=8.6 Hz, 1H), 7.47 (dt, J=8.5, 4.0 Hz, 3H), 7.19-7.14 (m, 2H), 5.78 (p, J=7.1 Hz, 2H), 5.21 (t, J=7.3 Hz, 1H), 3.91 (s, 3H), 2.56 (s, 2H), 1.50 (d, J=7.0 Hz, 3H).

### Example 58: Preparation of Compound 58

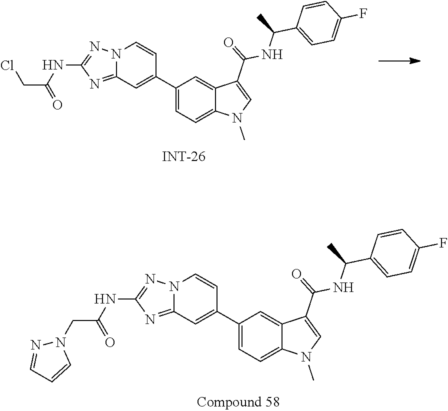 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)Cn5cccn5)nc4c3)cc12)c1ccc(F)cc1]

### Preparation of Compound 58

Intermediate INT-26 (101 mg, 0.2 mmol) was dissolved in acetonitrile (1 mL); pyrazole (27 mg, 0.4 mmol) and potassium carbonate (55 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 58 with a yield of 57%. MS (ESI, positive ion) m/z: 537.2 [M+H]⁺.

¹H NMR (400 MHz, DMSO-d₆) δ 11.24 (d, J=13.2 Hz, 1H), 8.91 (dd, J=7.0, 5.4 Hz, 1H), 8.56 (d, J=1.9 Hz, 1H), 8.38 (d, J=8.0 Hz, 1H), 8.23 (s, 1H), 7.96-7.91 (m, 1H), 7.83-7.72 (m, 2H), 7.68 (d, J=8.6 Hz, 1H), 7.53-7.43 (m, 3H), 7.23-7.12 (m, 2H), 5.79 (p, J=7.1 Hz, 2H), 5.22 (p, J=7.1 Hz, 1H), 3.92 (s, 3H), 2.55 (s, 2H), 1.50 (d, J=7.0 Hz, 3H).

### Example 59: Preparation of Compound 59

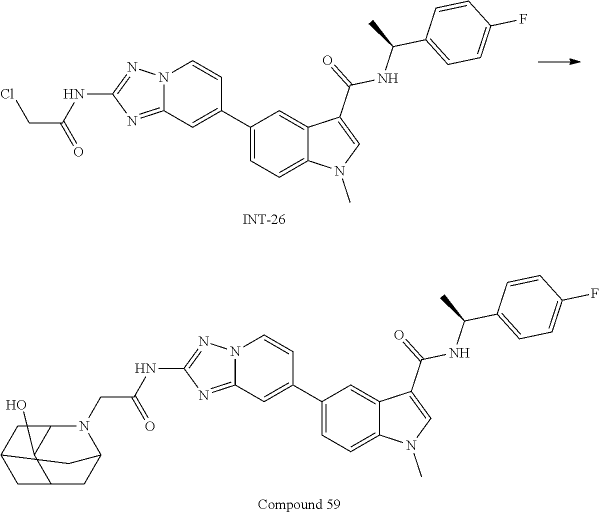 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5C6CC7CC(C6)C(O)C5C7)nc4c3)cc12)c1ccc(F)cc1]

### Preparation of Compound 59

Intermediate INT-26 (101 mg, 0.2 mmol) was dissolved in acetonitrile (1 mL); 3-amino-1-adamantanol (61 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 59 with a yield of 34%. MS (ESI, positive ion) m/z: 622.2 [M+H]⁺.

¹H NMR (400 MHz, DMSO-d₆) δ 10.44 (s, 1H), 8.89 (d, J=7.1 Hz, 1H), 8.55 (d, J=1.8 Hz, 1H), 8.38 (d, J=8.0 Hz, 1H), 8.23 (s, 1H), 7.90 (d, J=2.0 Hz, 1H), 7.74 (dd, J=8.7, 1.9 Hz, 1H), 7.67 (d, J=8.7 Hz, 1H), 7.47 (td, J=5.3, 2.4 Hz, 3H), 7.22-7.12 (m, 2H), 5.22 (p, J=7.2 Hz, 1H), 4.47 (s, 1H), 3.91 (s, 3H), 3.35 (dd, J=7.1, 3.7 Hz, 3H), 2.16 (d, J=4.4 Hz, 2H), 1.55-1.47 (m, 13H), 1.44 (d, J=3.4 Hz, 2H).

### Example 60: Preparation of Compound 60

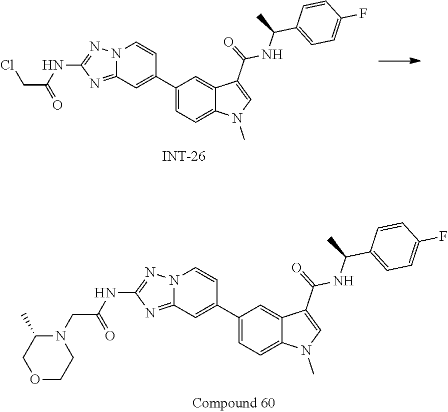 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOC[C@@H]5C)nc4c3)cc12)c1ccc(F)cc1]

### Preparation of Compound 60

Intermediate INT-26 (101 mg, 0.2 mmol) was dissolved in acetonitrile (1 mL), and (S)-3-methylmorpholine and cis-2,6-dimethylmorpholine (40 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 60 with a yield of 71%. MS (ESI, positive ion) m/z: 570.2 [M+H]⁺.

¹H NMR (400 MHz, DMSO-d₆) δ 10.43 (s, 1H), 8.89 (d, J=7.1 Hz, 1H), 8.55 (d, J=1.8 Hz, 1H), 8.37 (d, J=8.0 Hz, 1H), 8.23 (s, 1H), 7.90 (d, J=1.9 Hz, 1H), 7.74 (dd, J=8.7, 1.9 Hz, 1H), 7.67 (d, J=8.6 Hz, 1H), 7.50-7.45 (m, 3H), 7.22-7.12 (m, 2H), 5.23 (p, J=7.2 Hz, 1H), 3.92 (s, 3H), 3.73 (dt, J=11.3, 3.0 Hz, 1H), 3.66 (dd, J=11.1, 3.1 Hz, 1H), 3.57 (td, J=10.7, 2.5 Hz, 1H), 3.50 (d, J=16.3 Hz, 1H), 3.28-3.12 (m, 2H), 2.80 (dt, J=11.6, 2.7 Hz, 1H), 2.68-2.54 (m, 2H), 1.51 (d, J=7.0 Hz, 3H), 0.97 (d, J=6.3 Hz, 3H).

### Example 61: Preparation of Compound 61

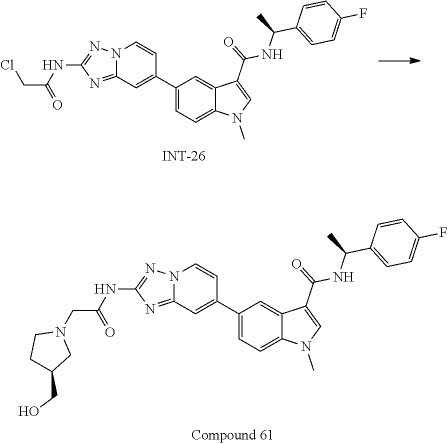 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@H](CO)C5)nc4c3)cc12)c1ccc(F)cc1]

### Preparation of Compound 61

Intermediate INT-26 (101 mg, 0.2 mmol) was dissolved in acetonitrile (1 mL); (S)-pyrrolidin-3-ylmethanol (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 61 with a yield of 58%. MS (ESI, positive ion) m/z: 570.2 [M+H]⁺.

¹H NMR (400 MHz, DMSO-d₆) δ 10.44 (s, 1H), 8.89 (d, J=7.1 Hz, 1H), 8.55 (d, J=1.8 Hz, 1H), 8.39 (d, J=8.0 Hz, 1H), 8.23 (s, 1H), 7.90 (d, J=1.9 Hz, 1H), 7.74 (dd, J=8.7, 1.9 Hz, 1H), 7.67 (d, J=8.6 Hz, 1H), 7.47 (td, J=5.5, 2.4 Hz, 3H), 7.22-7.12 (m, 2H), 5.22 (p, J=7.1 Hz, 1H), 4.60 (s, 1H), 3.91 (s, 3H), 3.35 (dd, J=7.1, 3.7 Hz, 4H), 2.74 (dd, J=9.1, 7.7 Hz, 1H), 2.64 (t, J=6.9 Hz, 2H), 2.45 (dd, J=9.1, 5.6 Hz, 1H), 2.30-2.25 (m, 1H), 1.93-1.75 (m, 1H), 1.50 (d, J=7.1 Hz, 3H), 1.44 (dt, J=12.9, 6.5 Hz, 1H).

### Example 62: Preparation of Compound 62

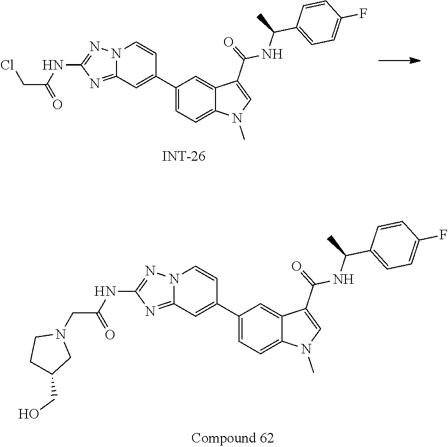 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC[C@@H](CO)C5)nc4c3)cc12)c1ccc(F)cc1]

### Preparation of Compound 62

Intermediate INT-26 (101 mg, 0.2 mmol) was dissolved in acetonitrile (1 mL); (R)-pyrrolidin-3-ylmethanol (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 62 with a yield of 54%. MS (ESI, positive ion) m/z: 570.2 [M+H]⁺.

¹H NMR (400 MHz, DMSO-d₆) δ 10.44 (s, 1H), 8.86 (d, J=7.1 Hz, 1H), 8.54 (d, J=1.8 Hz, 1H), 8.41 (d, J=8.0 Hz, 1H), 8.21 (s, 1H), 7.89 (d, J=1.9 Hz, 1H), 7.72 (dd, J=8.7, 1.8 Hz, 1H), 7.66 (d, J=8.6 Hz, 1H), 7.47 (td, J=6.9, 5.7, 2.2 Hz, 3H), 7.20-7.11 (m, 2H), 5.21 (p, J=7.2 Hz, 1H), 4.68 (s, 1H), 3.90 (s, 3H), 3.35 (dd, J=7.1, 3.7 Hz, 4H), 2.73 (dd, J=9.1, 7.8 Hz, 1H), 2.64 (t, J=6.9 Hz, 2H), 2.44 (dd, J=9.1, 5.7 Hz, 1H), 2.27 (t, J=7.8 Hz, 1H), 1.93-1.76 (m, 1H), 1.49 (d, J=7.0 Hz, 3H), 1.44 (dt, J=12.9, 6.4 Hz, 1H).

### Example 63: Preparation of Compound 63

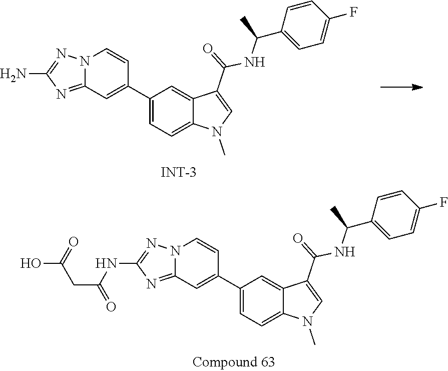 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)CC(=O)O)nc4c3)cc12)c1ccc(F)cc1]

### Preparation of Compound 63

Intermediate INT-3 (85 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 63 with a yield of 35%. MS (ESI, positive ion) m/z: 515.2 [M+H]⁺.

¹H NMR (400 MHz, DMSO-d₆) δ 11.03 (s, 1H), 8.86 (d, J=7.1 Hz, 1H), 8.55 (d, J=1.9 Hz, 1H), 8.39 (d, J=8.0 Hz, 1H), 8.23 (s, 1H), 7.89 (d, J=2.0 Hz, 1H), 7.74 (dd, J=8.6, 1.9 Hz, 1H), 7.67 (d, J=8.6 Hz, 1H), 7.47 (qd, J=6.9, 6.3, 2.1 Hz, 3H), 7.17 (t, J=8.9 Hz, 2H), 5.22 (p, J=7.2 Hz, 1H), 3.91 (s, 3H), 2.70 (s, 2H), 2.50 (s, 2H), 1.50 (d, J=7.0 Hz, 3H).

### Example 64: Preparation of Compound 64

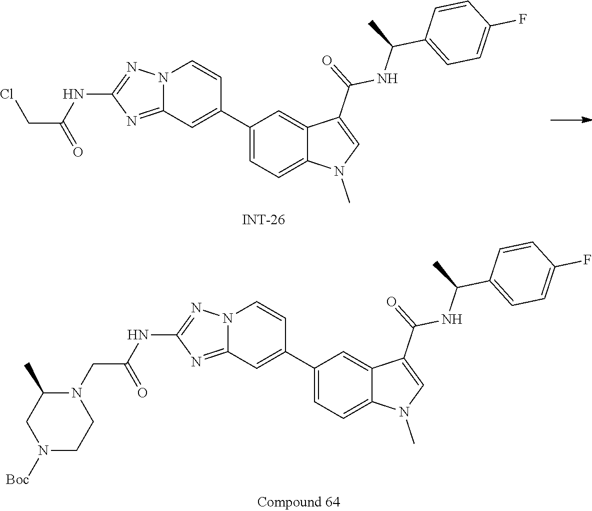 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCN(C(=O)OC(C)(C)C)C[C@H]5C)nc4c3)cc12)c1ccc(F)cc1]

### Preparation of Compound 64

Intermediate INT-26 (101 mg, 0.2 mmol) was dissolved in acetonitrile (1 mL), and (R)-4-Boc-2-methylpiperazine (80 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 64 with a yield of 61%. MS (ESI, positive ion) m/z: 669.2 [M+H]⁺.

¹H NMR (400 MHz, DMSO-d₆) δ 10.45 (s, 1H), 8.88 (d, J=7.1 Hz, 1H), 8.55 (d, J=1.9 Hz, 1H), 8.36 (d, J=8.1 Hz, 1H), 8.22 (s, 1H), 7.90 (d, J=1.9 Hz, 1H), 7.74 (dd, J=8.7, 1.9 Hz, 1H), 7.67 (d, J=8.7 Hz, 1H), 7.49-7.45 (m, 3H), 7.17 (t, J=8.9 Hz, 2H), 5.22 (p, J=7.1 Hz, 1H), 3.91 (s, 3H), 3.67 (d, J=13.0 Hz, 2H), 3.46 (d, J=15.7 Hz, 1H), 3.09 (d, J=11.9 Hz, 1H), 2.83 (d, J=11.5 Hz, 1H), 2.62 (s, 1H), 1.50 (d, J=7.1 Hz, 3H), 1.41 (d, J=7.3 Hz, 12H), 1.03 (d, J=6.2 Hz, 3H).

### Example 65: Preparation of Compound 65

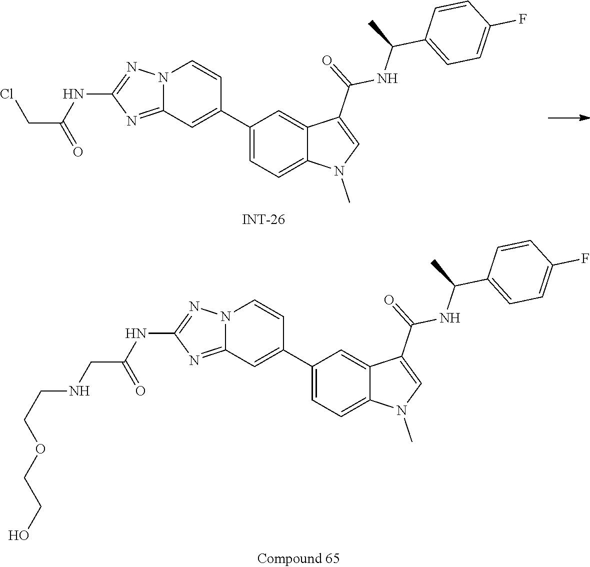 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNCCOCCO)nc4c3)cc12)c1ccc(F)cc1]

### Preparation of Compound 65

Intermediate INT-26 (101 mg, 0.2 mmol) was dissolved in acetonitrile (1 mL); diglycolamine (42 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 65 with a yield of 71%. MS (ESI, positive ion) m/z: 574.2 [M+H]⁺.

¹H NMR (400 MHz, DMSO-d₆) δ 8.92 (dd, J=23.7, 7.1 Hz, 1H), 8.56 (d, J=1.9 Hz, 1H), 8.37 (d, J=8.0 Hz, 1H), 8.23 (s, 1H), 7.89 (t, J=1.3 Hz, 1H), 7.75 (dd, J=8.7, 2.0 Hz, 1H), 7.71-7.64 (m, 1H), 7.56-7.44 (m, 3H), 7.22-7.13 (m, 2H), 5.23 (p, J=7.2 Hz, 1H), 3.92 (s, 3H), 3.58-3.43 (m, 8H), 2.75 (t, J=5.5 Hz, 2H), 1.51 (d, J=7.0 Hz, 3H).

### Example 66: Preparation of Compound 66

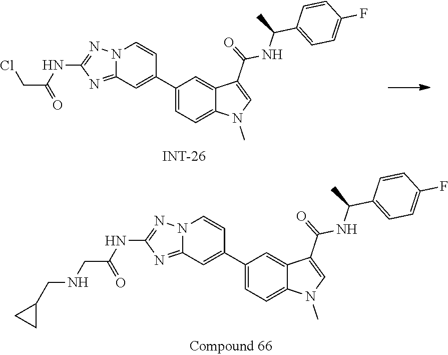 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNCC5CC5)nc4c3)cc12)c1ccc(F)cc1]

### Preparation of Compound 66

Intermediate INT-26 (101 mg, 0.2 mmol) was dissolved in acetonitrile (1 mL); cyclopropylmethylamine (28 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 66 with a yield of 75%. MS (ESI, positive ion) m/z: 540.2 [M+H]⁺.

¹H NMR (400 MHz, DMSO-d₆) δ 10.43 (s, 1H), 8.93 (dd, J=13.2, 7.1 Hz, 1H), 8.56 (dd, J=4.8, 1.8 Hz, 1H), 8.48 (t, J=8.1 Hz, 1H), 8.31 (d, J=6.8 Hz, 1H), 7.97 (dd, J=24.3, 1.9 Hz, 1H), 7.75 (dd, J=8.7, 1.9 Hz, 1H), 7.68 (d, J=8.7 Hz, 2H), 7.48 (d, J=2.9 Hz, 2H), 7.21-7.12 (m, 2H), 5.20 (q, J=7.3 Hz, 1H), 4.44 (t, J=5.4 Hz, 1H), 3.91 (s, 3H), 3.46 (s, 1H), 2.88 (d, J=7.3 Hz, 1H), 2.74 (t, J=5.5 Hz, 2H), 1.50 (d, J=7.1 Hz, 3H), 1.17-0.85 (m, 1H), 0.63-0.16 (m, 4H).

### Example 67: Preparation of Compound 67

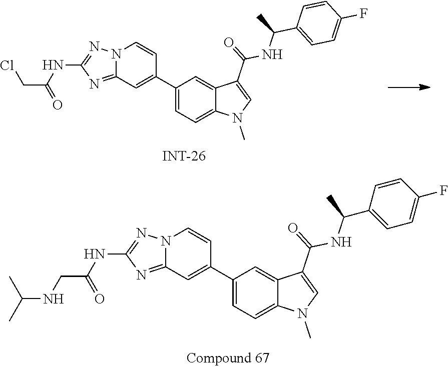

### Preparation of Compound 67

Intermediate INT-26 (101 mg, 0.2 mmol) was dissolved in acetonitrile (1 mL); isopropylamine (23 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 67 with a yield of 65%. MS (ESI, positive ion) m/z: 528.2 [M+H]⁺.

¹H NMR (400 MHz, DMSO-d₆) δ 8.91 (dd, J=22.1, 7.1 Hz, 1H), 8.55 (d, J=1.9 Hz, 1H), 8.36 (d, J=8.0 Hz, 1H), 8.22 (s, 1H), 7.90 (d, J=1.9 Hz, 1H), 7.74 (dd, J=8.7, 1.9 Hz, 1H), 7.68 (d, J=8.7 Hz, 1H), 7.47 (td, J=5.5, 2.5 Hz, 3H), 7.22-7.13 (m, 2H), 5.24 (h, J=7.3, 6.7 Hz, 1H), 3.92 (s, 3H), 3.51-3.39 (m, 2H), 2.78 (p, J=6.2 Hz, 1H), 1.51 (d, J=7.0 Hz, 3H), 1.04 (d, J=6.2 Hz, 6H).

### Example 68: Preparation of Compound 68

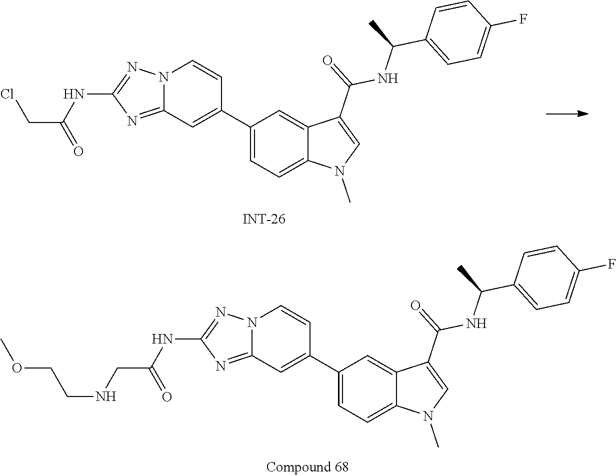 [SMILES: C[C@H](NC(=O)c1cn(C)c2ccc(-c3ccn4nc(NC(=O)CCl)nc4c3)cc12)c1ccc(F)cc1 ; "INT-26" ; COCCNCC(=O)Nc1nc2cc(-c3ccc4c(c3)c(C(=O)N[C@@H](C)c3ccc(F)cc3)cn4C)ccn2n1]

### Preparation of Compound 68

Intermediate INT-26 (101 mg, 0.2 mmol) was dissolved in acetonitrile (1 mL); 2-methoxyethylamine (30 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 68 with a yield of 75%. MS (ESI, positive ion) m/z: 544.2 [M+H]⁺.

¹H NMR (400 MHz, DMSO-d₆) δ 10.43 (s, 1H), 8.89 (d, J=7.2 Hz, 1H), 8.55 (d, J=1.8 Hz, 1H), 8.38 (d, J=7.9 Hz, 1H), 8.23 (s, 1H), 7.90 (d, J=2.1 Hz, 1H), 7.73 (d, J=1.9 Hz, 1H), 7.67 (d, J=8.7 Hz, 1H), 7.48 (dd, J=5.6, 3.2 Hz, 3H), 7.19-7.16 (m, 2H), 5.21 (d, J=7.4 Hz, 1H), 4.44 (t, J=5.4 Hz, 1H), 3.91 (s, 3H), 3.46 (s, 1H), 3.43 (t, J=5.5 Hz, 3H), 3.27-3.24 (m, 3H), 2.74 (t, J=5.5 Hz, 2H), 1.50 (d, J=7.1 Hz, 3H).

### Example 69: Preparation of Compound 69

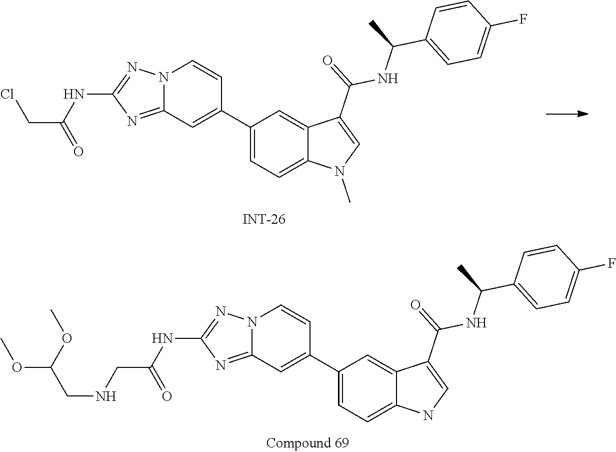 [SMILES: C[C@H](NC(=O)c1cn(C)c2ccc(-c3ccn4nc(NC(=O)CCl)nc4c3)cc12)c1ccc(F)cc1 ; "INT-26" ; COC(CNCC(=O)Nc1nc2cc(-c3ccc4[nH]cc(C(=O)N[C@@H](C)c5ccc(F)cc5)c4c3)ccn2n1)OC]

### Preparation of Compound 69

Intermediate INT-26 (101 mg, 0.2 mmol) was dissolved in acetonitrile (1 mL); aminoacetaldehyde dimethyl acetal (42 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 69 with a yield of 66%. MS (ESI, positive ion) m/z: 574.2 [M+H]⁺.

¹H NMR (400 MHz, DMSO-d₆) δ 10.43 (s, 1H), 8.89 (d, J=7.1 Hz, 1H), 8.55 (d, J=1.9 Hz, 1H), 8.38 (d, J=8.0 Hz, 1H), 8.23 (s, 1H), 7.90 (d, J=2.0 Hz, 1H), 7.75 (dd, J=8.7, 1.9 Hz, 1H), 7.68 (d, J=8.6 Hz, 1H), 7.47 (td, J=5.5, 2.5 Hz, 3H), 7.23-7.13 (m, 2H), 5.22 (p, J=7.1 Hz, 1H), 4.44 (t, J=5.4 Hz, 1H), 3.92 (s, 3H), 3.46 (s, 1H), 3.31 (s, 6H), 3.29-3.26 (m, 2H), 2.69 (d, J=5.4 Hz, 2H), 1.50 (d, J=7.1 Hz, 3H).

### Example 70: Preparation of Compound 70

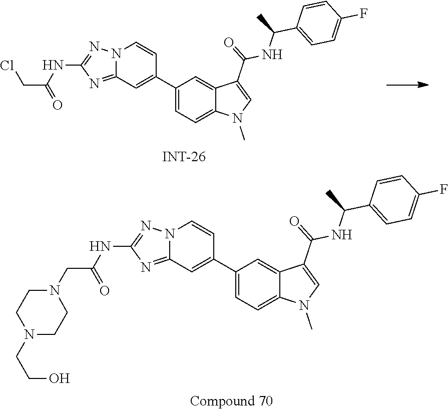 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCN(CCO)CC5)nc4c3)cc12)c1ccc(F)cc1]

### Preparation of Compound 70

Intermediate INT-26 (101 mg, 0.2 mmol) was dissolved in acetonitrile (1 mL); N-hydroxyethylpiperazine (52 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 70 with a yield of 56%. MS (ESI, positive ion) m/z: 599.2 [M+H]⁺.

¹H NMR (400 MHz, DMSO-d₆) δ 10.43 (s, 1H), 8.89 (d, J=7.1 Hz, 1H), 8.55 (d, J=1.8 Hz, 1H), 8.38 (d, J=8.0 Hz, 1H), 8.23 (s, 1H), 7.90 (d, J=2.0 Hz, 1H), 7.74 (dd, J=8.6, 1.9 Hz, 1H), 7.67 (d, J=8.6 Hz, 1H), 7.51-7.42 (m, 3H), 7.22-7.12 (m, 2H), 5.22 (p, J=7.1 Hz, 1H), 4.39 (t, J=5.4 Hz, 1H), 3.91 (s, 3H), 3.51 (q, J=6.1 Hz, 2H), 3.23 (s, 2H), 2.56 (s, 3H), 2.47 (d, J=7.7 Hz, 3H), 2.40 (t, J=6.3 Hz, 2H), 1.50 (d, J=7.0 Hz, 3H).

### Example 71: Preparation of Compound 71

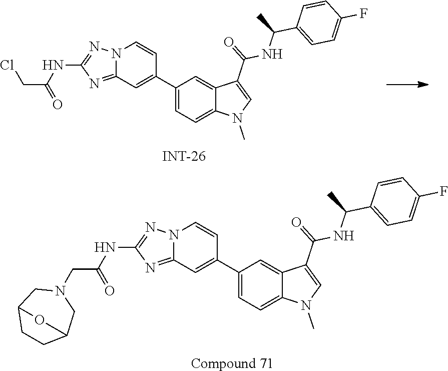 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CC6CCC(C5)O6)nc4c3)cc12)c1ccc(F)cc1 ; "Compound 71"]

### Preparation of Compound 71

Intermediate INT-26 (101 mg, 0.2 mmol) was dissolved in acetonitrile (1 mL); 8-oxa-3-azabicyclo[3.2.1]octane hydrochloride (45 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 71 with a yield of 56%. MS (ESI, positive ion) m/z: 582.2 [M+H]⁺.

¹H NMR (400 MHz, DMSO-d₆) δ 10.41 (s, 1H), 8.93-8.86 (m, 1H), 8.55 (d, J=1.9 Hz, 1H), 8.38 (d, J=8.0 Hz, 1H), 8.23 (s, 1H), 7.92 (dd, J=2.0, 0.9 Hz, 1H), 7.75 (dd, J=8.7, 1.9 Hz, 1H), 7.68 (d, J=8.6 Hz, 1H), 7.49-7.45 (m, 3H), 7.20-7.15 (m, 2H), 5.22 (p, J=7.1 Hz, 1H), 4.25 (s, 2H), 3.92 (s, 4H), 3.22 (s, 2H), 2.64 (d, J=10.7 Hz, 2H), 2.47 (dd, J=11.1, 2.1 Hz, 3H), 1.77 (s, 2H), 1.50 (d, J=7.1 Hz, 3H).

### Example 72: Preparation of Compound 72

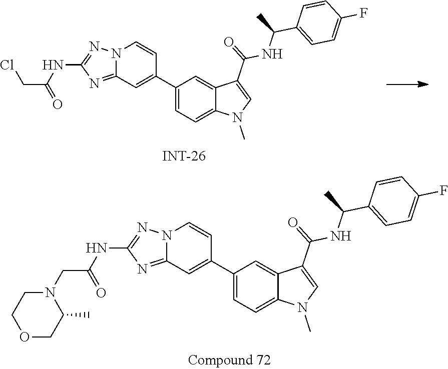 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOC[C@H]5C)nc4c3)cc12)c1ccc(F)cc1]

### Preparation of Compound 72

Intermediate INT-26 (101 mg, 0.2 mmol) was dissolved in acetonitrile (1 mL); (R)-3-methylmorpholine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 72 with a yield of 76%. MS (ESI, positive ion) m/z: 570.2 [M+H]⁺.

¹H NMR (400 MHz, DMSO-d₆) δ 10.43 (s, 1H), 8.89 (dd, J=7.1, 0.8 Hz, 1H), 8.55 (d, J=1.8 Hz, 1H), 8.37 (d, J=8.0 Hz, 1H), 8.23 (s, 1H), 7.90 (dd, J=1.9, 0.9 Hz, 1H), 7.74 (dd, J=8.7, 1.9 Hz, 1H), 7.67 (d, J=8.7 Hz, 1H), 7.52-7.43 (m, 3H), 7.22-7.12 (m, 2H), 5.23 (p, J=7.2 Hz, 1H), 3.91 (s, 3H), 3.73 (dt, J=11.2, 3.1 Hz, 1H), 3.66 (dd, J=11.0, 3.1 Hz, 1H), 3.62-3.53 (m, 1H), 3.53-3.42 (m, 1H), 3.28-3.12 (m, 2H), 2.80 (dt, J=11.7, 2.7 Hz, 1H), 2.70-2.54 (m, 2H), 1.50 (d, J=7.1 Hz, 3H), 0.97 (d, J=6.3 Hz, 3H).

### Example 73: Preparation of Compound 73

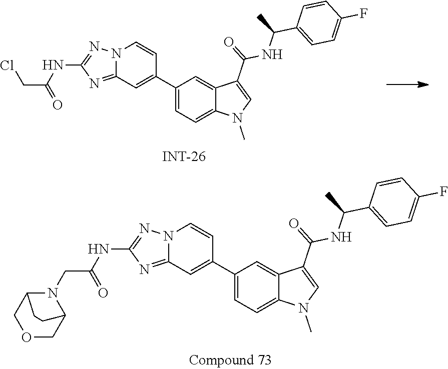 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5C6CCC5COC6)nc4c3)cc12)c1ccc(F)cc1]

### Preparation of Compound 73

Intermediate INT-26 (101 mg, 0.2 mmol) was dissolved in acetonitrile (1 mL); nortropane (45 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 73 with a yield of 65%. MS (ESI, positive ion) m/z: 582.2 [M+H]⁺.

¹H NMR (400 MHz, DMSO-d₆) δ 10.45 (s, 1H), 8.90 (d, J=7.2 Hz, 1H), 8.55 (d, J=1.9 Hz, 1H), 8.38 (d, J=8.0 Hz, 1H), 8.22 (s, 1H), 7.91 (d, J=2.0 Hz, 1H), 7.74 (dd, J=8.7, 1.9 Hz, 1H), 7.68 (d, J=8.6 Hz, 1H), 7.48 (dd, J=6.5, 2.7 Hz, 3H), 7.19-7.15 (m, 2H), 5.22 (t, J=7.3 Hz, 1H), 3.91 (s, 4H), 3.69 (d, J=10.3 Hz, 2H), 3.49 (dd, J=10.7, 2.1 Hz, 2H), 3.15 (s, 3H), 1.90 (dd, J=9.0, 4.6 Hz, 2H), 1.82-1.76 (m, 2H), 1.50 (d, J=7.0 Hz, 3H).

### Example 74: Preparation of Compound 74

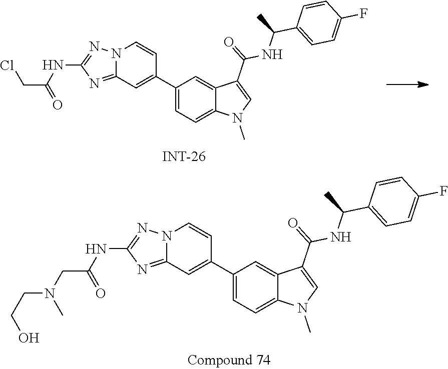 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN(C)CCO)nc4c3)cc12)c1ccc(F)cc1]

### Preparation of Compound 74

Intermediate INT-26 (101 mg, 0.2 mmol) was dissolved in acetonitrile (1 mL); N-methyl-2-hydroxyethylamine (30 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 74 with a yield of 55%. MS (ESI, positive ion) m/z: 544.2 [M+H]⁺.

¹H NMR (400 MHz, DMSO-d₆) δ 10.51 (s, 1H), 8.89 (d, J=7.1 Hz, 1H), 8.60-8.52 (m, 1H), 8.37 (t, J=7.5 Hz, 1H), 8.22 (d, J=7.4 Hz, 1H), 7.90 (d, J=1.9 Hz, 1H), 7.74 (dd, J=8.7, 1.9 Hz, 1H), 7.71-7.62 (m, 1H), 7.51-7.43 (m, 3H), 7.23-7.12 (m, 2H), 5.22 (p, J=7.3 Hz, 1H), 4.71 (s, 1H), 3.91 (d, J=4.8 Hz, 3H), 3.56-3.42 (m, 3H), 2.63-2.53 (m, 2H), 2.35 (s, 3H), 2.30 (s, 1H), 1.50 (d, J=7.1 Hz, 3H).

### Example 75: Preparation of Compound 75

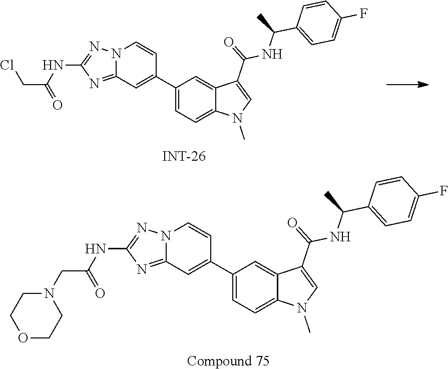 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CN5CCOCC5)nc4c3)cc12)c1ccc(F)cc1 ; "Compound 75"]

### Preparation of Compound 75

Intermediate INT-26 (101 mg, 0.2 mmol) was dissolved in acetonitrile (1 mL); morpholine (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 75 with a yield of 85%. MS (ESI, positive ion) m/z: 556.2 [M+H]⁺.

¹H NMR (400 MHz, DMSO-d₆) δ 10.54 (s, 1H), 8.89 (d, J=7.1 Hz, 1H), 8.55 (d, J=1.9 Hz, 1H), 8.38 (d, J=8.1 Hz, 1H), 8.23 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 5.22 (p, J=7.2 Hz, 1H), 3.91 (s, 3H), 3.64 (t, J=4.6 Hz, 4H), 3.27 (s, 2H), 2.56 (dd, J=5.4, 3.4 Hz, 4H), 1.50 (d, J=7.0 Hz, 3H).

### Example 76: Preparation of Compound 76

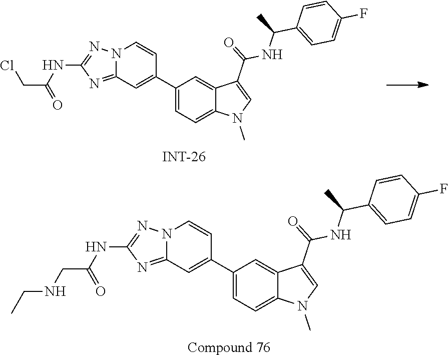 [SMILES: C[C@H](NC(=O)c1cn(C)c2ccc(-c3ccn4nc(NC(=O)CCl)nc4c3)cc12)c1ccc(F)cc1 ; "INT-26" ; CCNCC(=O)Nc1nc2cc(-c3ccc4c(c3)c(C(=O)N[C@@H](C)c3ccc(F)cc3)cn4C)ccn2n1 ; "Compound 76"]

### Preparation of Compound 76

Intermediate INT-26 (101 mg, 0.2 mmol) was dissolved in acetonitrile (1 mL); ethylamine hydrochloride (32 mg, 0.4 mmol) and DIPEA (103 mg, 0.8 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 76 with a yield of 68%. MS (ESI, positive ion) m/z: 514.2 [M+H]⁺.

¹H NMR (400 MHz, DMSO-d₆) δ 10.54 (s, 1H), 8.89 (d, J=7.1 Hz, 1H), 8.55 (d, J=1.9 Hz, 1H), 8.38 (d, J=8.1 Hz, 1H), 8.23 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 4.97 (m, 1H), 3.91 (s, 3H), 3.44 (s, 1H), 3.22 (s, 2H), 2.63 (dd, J=6.6, 1.8 Hz, 2H), 1.56 (d, J=5.4 Hz, 3H), 1.10 (dd, J=7.6, 2.8 Hz, 3H).

### Example 77: Preparation of Compound 77

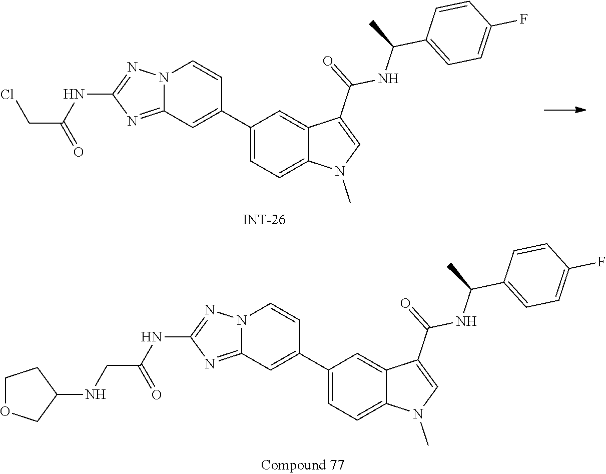 [SMILES: C[C@H](NC(=O)c1cn(C)c2ccc(-c3ccn4nc(NC(=O)CCl)nc4c3)cc12)c1ccc(F)cc1 ; "INT-26" ; C[C@H](NC(=O)c1cn(C)c2ccc(-c3ccn4nc(NC(=O)CNC5CCOC5)nc4c3)cc12)c1ccc(F)cc1]

### Preparation of Compound 77

Intermediate INT-26 (101 mg, 0.2 mmol) was dissolved in acetonitrile (1 mL); 3-aminotetrahydrofuran (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 77 with a yield of 61%. MS (ESI, positive ion) m/z: 556.2 [M+H]⁺.

¹H NMR (400 MHz, DMSO-d₆) δ 10.54 (s, 1H), 8.89 (d, J=7.1 Hz, 1H), 8.55 (d, J=1.9 Hz, 1H), 8.38 (d, J=8.1 Hz, 1H), 8.23 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 5.13 (s, 1H), 4.97 (m, 1H), 3.91-3.75 (m, 7H), 3.01-2.95 (m, 1H), 2.63 (dd, J=6.6, 1.8 Hz, 2H), 2.06-1.83 (m, 2H), 1.56 (d, J=5.4 Hz, 3H).

### Example 78: Preparation of Compound 78

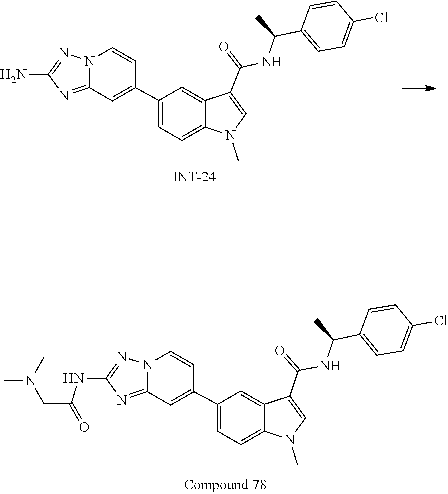 [SMILES: C[C@H](NC(=O)c1cn(C)c2ccc(-c3ccn4nc(N)nc4c3)cc12)c1ccc(Cl)cc1 ; "INT-24" ; C[C@H](NC(=O)c1cn(C)c2ccc(-c3ccn4nc(NC(=O)CN(C)C)nc4c3)cc12)c1ccc(Cl)cc1]

### Preparation of Compound 78

Intermediate INT-24 (89 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 78 with a yield of 49%. MS (ESI, positive ion) m/z: 530.2 [M+H]⁺.

¹H NMR (400 MHz, DMSO-d₆) δ 10.44 (s, 1H), 8.88 (dd, J=7.1, 0.8 Hz, 1H), 8.55 (d, J=1.8 Hz, 1H), 8.39 (d, J=7.9 Hz, 1H), 8.23 (s, 1H), 7.90 (dd, J=2.0, 0.9 Hz, 1H), 7.74 (dd, J=8.7, 1.9 Hz, 1H), 7.67 (d, J=8.6 Hz, 1H), 7.50-7.45 (m, 3H), 7.43-7.39 (m, 2H), 5.21 (p, J=7.1 Hz, 1H), 3.92 (s, 3H), 3.21 (s, 2H), 2.33 (s, 6H), 1.50 (d, J=7.1 Hz, 3H).

### Example 79: Preparation of Compound 79

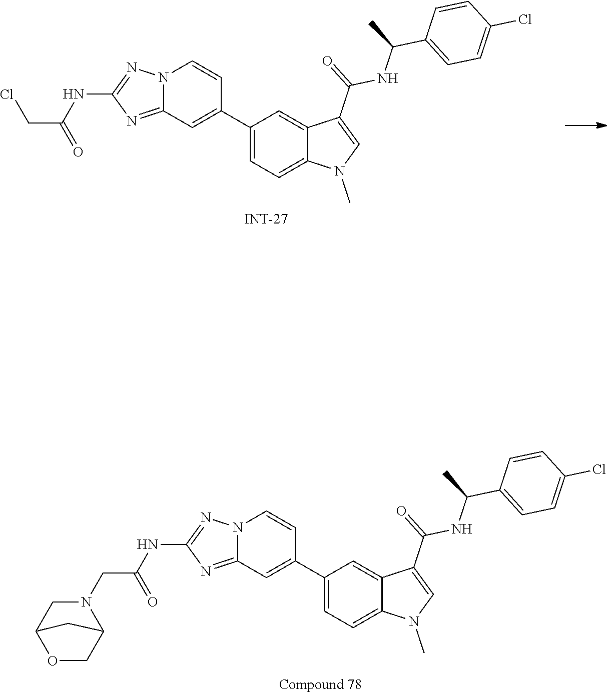 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CC6CC5CO6)nc4c3)cc12)c1ccc(Cl)cc1]

### Preparation of Compound 79

Intermediate INT-27 (104 mg, 0.2 mmol) was dissolved in acetonitrile (1 mL), and 2-oxa-5-azabicyclo[2.2.1]heptane (39 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 79 with a yield of 71%. MS (ESI, positive ion) m/z: 584.2 [M+H]⁺.

¹H NMR (400 MHz, DMSO-d₆) δ 10.39 (s, 1H), 8.88 (d, J=7.1 Hz, 1H), 8.55 (d, J=1.8 Hz, 1H), 8.39 (d, J=7.9 Hz, 1H), 8.23 (s, 1H), 7.90 (d, J=1.9 Hz, 1H), 7.74 (dd, J=8.6, 1.9 Hz, 1H), 7.67 (d, J=8.6 Hz, 1H), 7.49-7.45 (m, 3H), 7.42-7.39 (m, 2H), 5.26-5.15 (m, 1H), 4.39 (t, J=2.1 Hz, 1H), 3.90 (d, J=11.3 Hz, 4H), 3.62 (s, 1H), 3.58 (dd, J=7.7, 1.8 Hz, 1H), 3.48 (d, J=5.2 Hz, 2H), 2.96 (dd, J=9.9, 1.7 Hz, 1H), 2.57 (d, J=10.0 Hz, 1H), 1.87 (dd, J=9.7, 2.2 Hz, 1H), 1.68-1.61 (m, 1H), 1.50 (d, J=7.1 Hz, 3H).

### Example 80: Preparation of Compound 80

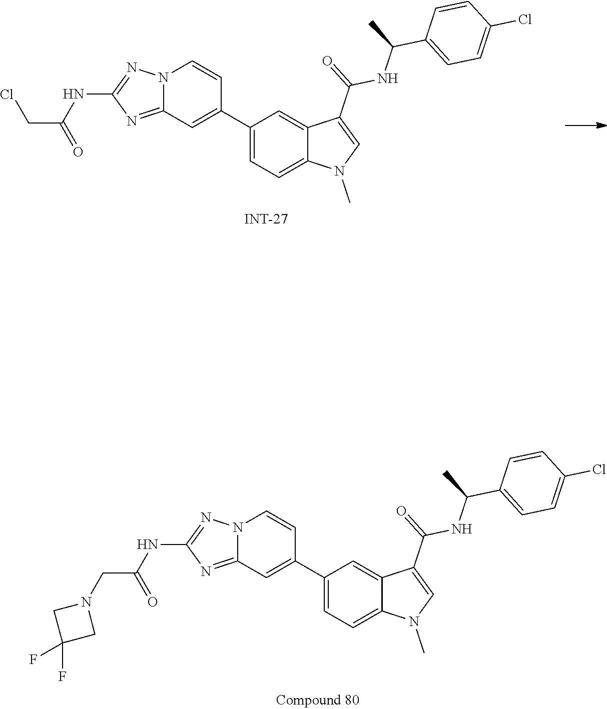 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CC(F)(F)C5)nc4c3)cc12)c1ccc(Cl)cc1]

### Preparation of Compound 80

Intermediate INT-27 (104 mg, 0.2 mmol) was dissolved in acetonitrile (1 mL); 3,3-difluoroazetidine (37 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 80 with a yield of 65%. MS (ESI, positive ion) m/z: 578.2 [M+H]⁺.

¹H NMR (400 MHz, DMSO) δ 10.77 (s, 1H), 8.89 (t, J=10.7 Hz, 1H), 8.55 (s, 1H), 8.41 (d, J=7.9 Hz, 1H), 8.24 (s, 1H), 7.91 (d, J=11.7 Hz, 1H), 7.79-7.64 (m, 2H), 7.47 (dd, J=9.4, 5.0 Hz, 3H), 7.41 (d, J=8.5 Hz, 2H), 5.20 (p, J=7.0 Hz, 1H), 3.92 (s, 3H), 3.80 (t, J=12.5 Hz, 4H), 3.61 (s, 2H), 1.50 (d, J=7.0 Hz, 3H).

### Example 81: Preparation of Compound 81

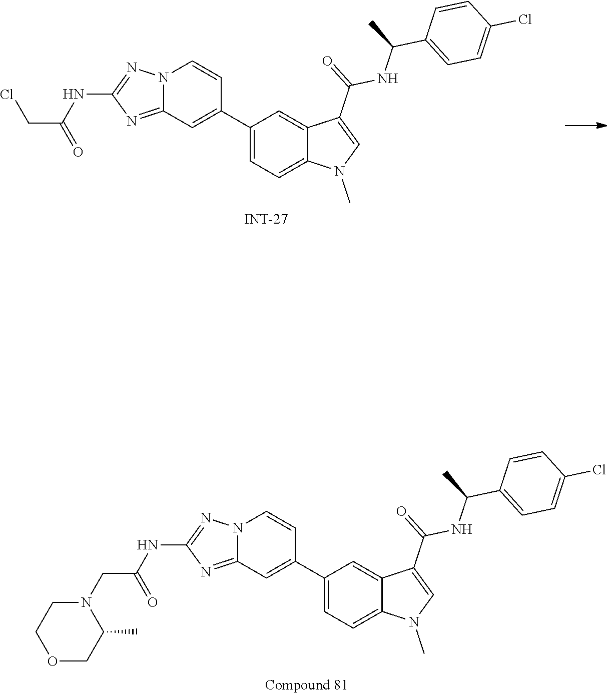 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CCOC[C@H]5C)nc4c3)cc12)c1ccc(Cl)cc1]

### Preparation of Compound 81

Intermediate INT-27 (104 mg, 0.2 mmol) was dissolved in acetonitrile (1 mL); (R)-3-methylmorpholine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 81 with a yield of 73%. MS (ESI, positive ion) m/z: 586.2 [M+H]⁺.

¹H NMR (400 MHz, DMSO-d₆) δ 10.43 (s, 1H), 8.89 (d, J=7.1 Hz, 1H), 8.55 (d, J=1.8 Hz, 1H), 8.39 (d, J=7.9 Hz, 1H), 8.24 (s, 1H), 7.90 (d, J=2.0 Hz, 1H), 7.74 (dd, J=8.7, 1.9 Hz, 1H), 7.68 (d, J=8.7 Hz, 1H), 7.49-7.44 (m, 3H), 7.43-7.38 (m, 2H), 5.25-5.17 (m, 1H), 3.92 (s, 3H), 3.76-3.70 (m, 1H), 3.66 (dd, J=10.9, 3.1 Hz, 1H), 3.61-3.46 (m, 2H), 3.22-3.15 (m, 1H), 2.82-2.78 (m, 1H), 2.66-2.54 (m, 2H), 1.50 (d, J=7.1 Hz, 3H), 0.97 (d, J=6.3 Hz, 3H).

### Example 82: Preparation of Compound 82

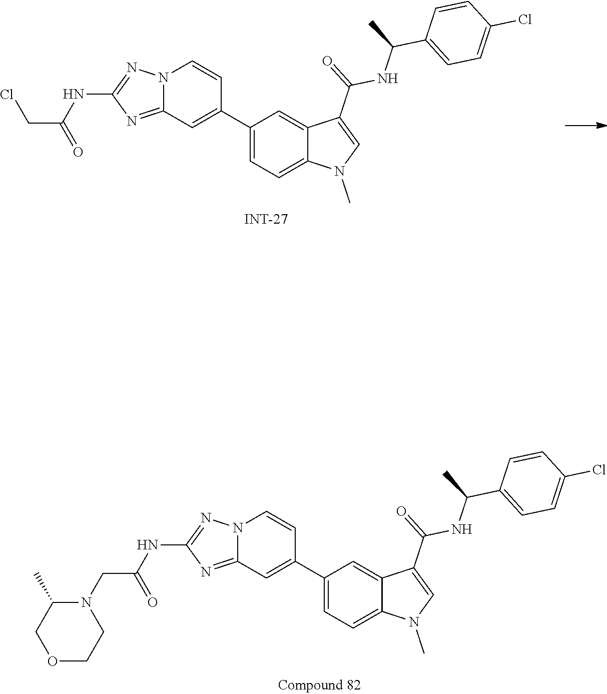 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CCOC[C@@H]5C)nc4c3)cc12)c1ccc(Cl)cc1]

### Preparation of Compound 82

Intermediate INT-27 (104 mg, 0.2 mmol) was dissolved in acetonitrile (1 mL), and (S)-3-methylmorpholine and cis-2,6-dimethylmorpholine (40 mg, 0.4 mmol) were added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 82 with a yield of 68%. MS (ESI, positive ion) m/z: 586.2 [M+H]⁺.

¹H NMR (400 MHz, DMSO) δ 10.45 (s, 1H), 8.89 (d, J=7.1 Hz, 1H), 8.55 (s, 1H), 8.43 (t, J=13.8 Hz, 1H), 8.24 (s, 1H), 7.90 (s, 1H), 7.71 (dd, J=25.0, 8.6 Hz, 2H), 7.50-7.44 (m, 3H), 7.41 (d, J=8.4 Hz, 2H), 5.20 (p, J=6.9 Hz, 1H), 3.92 (s, 3H), 3.69 (dd, J=27.9, 9.8 Hz, 2H), 3.61-3.46 (m, 2H), 3.34-3.10 (m, 2H), 2.79 (d, J=11.7 Hz, 1H), 2.60 (dd, J=18.8, 8.3 Hz, 2H), 1.50 (d, J=7.0 Hz, 3H), 0.96 (d, J=6.3 Hz, 3H).

### Example 83: Preparation of Compound 83

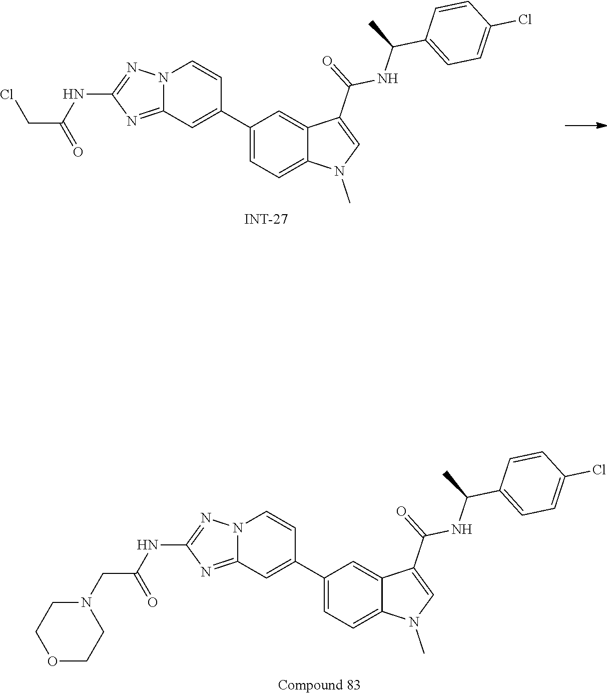 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CCOCC5)nc4c3)cc12)c1ccc(Cl)cc1]

### Preparation of Compound 83

Intermediate INT-27 (104 mg, 0.2 mmol) was dissolved in acetonitrile (1 mL); morpholine (35 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 83 with a yield of 82%. MS (ESI, positive ion) m/z: 572.2 [M+H]⁺.

¹H NMR (400 MHz, DMSO-d₆) δ 10.53 (s, 1H), 8.89 (d, J=7.1 Hz, 1H), 8.54 (d, J=2.0 Hz, 1H), 8.40 (d, J=7.9 Hz, 1H), 8.23 (s, 1H), 7.90 (d, J=1.9 Hz, 1H), 7.74 (dd, J=8.6, 1.9 Hz, 1H), 7.67 (d, J=8.7 Hz, 1H), 7.51-7.44 (m, 3H), 7.44-7.37 (m, 2H), 5.20 (p, J=7.2 Hz, 1H), 3.91 (s, 3H), 3.64 (t, J=4.6 Hz, 4H), 3.27 (s, 2H), 2.58-2.54 (m, 4H), 1.50 (d, J=7.1 Hz, 3H).

### Example 84: Preparation of Compound 84

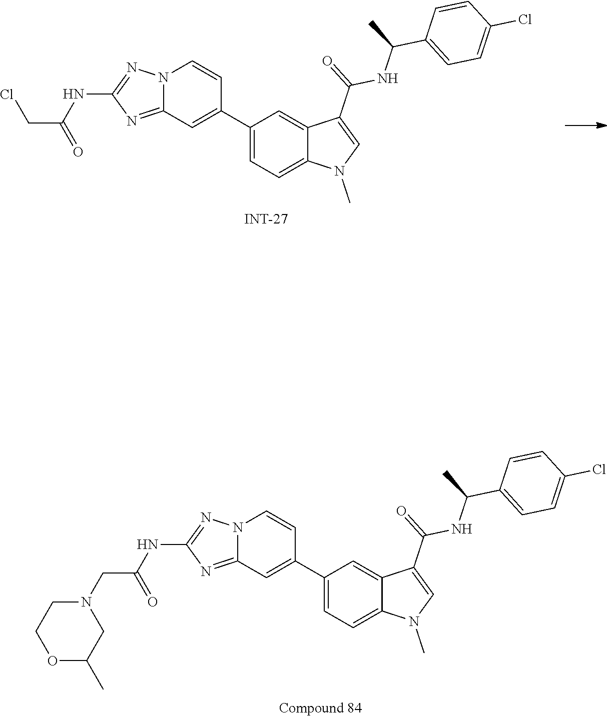 [SMILES: C[C@H](NC(=O)c1cn(C)c2ccc(-c3ccn4nc(NC(=O)CCl)nc4c3)cc12)c1ccc(Cl)cc1 ; "INT-27" ; CC1CN(CC(=O)Nc2nc3cc(-c4ccc5c(c4)c(C(=O)N[C@@H](C)c4ccc(Cl)cc4)cn5C)ccn3n2)CCO1]

### Preparation of Compound 84

Intermediate INT-27 (104 mg, 0.2 mmol) was dissolved in acetonitrile (1 mL); 3-(hydroxymethyl)piperidine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 84 with a yield of 66%. MS (ESI, positive ion) m/z: 586.2 [M+H]⁺.

¹H NMR (400 MHz, DMSO-d₆) δ 10.52 (s, 1H), 8.88 (d, J=7.1 Hz, 1H), 8.54 (d, J=1.8 Hz, 1H), 8.40 (d, J=7.9 Hz, 1H), 8.23 (s, 1H), 7.90 (d, J=1.9 Hz, 1H), 7.74 (dd, J=8.6, 1.9 Hz, 1H), 7.67 (d, J=8.7 Hz, 1H), 7.49-7.44 (m, 3H), 7.40 (d, J=8.6 Hz, 2H), 5.20 (p, J=7.2 Hz, 1H), 3.91 (s, 3H), 3.77 (dt, J=10.0, 2.4 Hz, 1H), 3.58 (ddd, J=13.8, 8.9, 2.8 Hz, 2H), 3.26 (s, 2H), 2.82 (dt, J=11.1, 2.1 Hz, 1H), 2.75 (dd, J=11.1, 2.1 Hz, 1H), 2.28 (td, J=11.3, 3.3 Hz, 1H), 1.98 (dd, J=11.1, 9.9 Hz, 1H), 1.50 (d, J=7.1 Hz, 3H), 1.07 (d, J=6.2 Hz, 3H).

### Example 85: Preparation of Compound 85

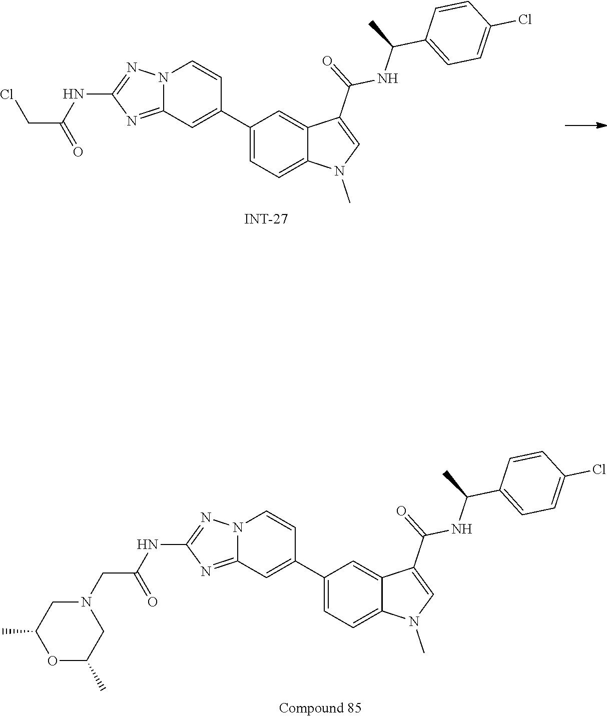 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@@H]1CN(CC(=O)Nc2nc3cc(-c4ccc5c(c4)c(C(=O)N[C@@H](C)c4ccc(Cl)cc4)cn5C)ccn3n2)C[C@H](C)O1]

### Preparation of Compound 85

Intermediate INT-27 (104 mg, 0.2 mmol) was dissolved in acetonitrile (1 mL), and cis-2,6-dimethylmorpholine (46 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 85 with a yield of 64%. MS (ESI, positive ion) m/z: 600.2 [M+H]⁺.

¹H NMR (400 MHz, DMSO-d₆) δ 10.50 (s, 1H), 8.86 (d, J=7.1 Hz, 1H), 8.52 (d, J=1.9 Hz, 1H), 8.39 (d, J=7.9 Hz, 1H), 8.21 (s, 1H), 7.88 (d, J=2.0 Hz, 1H), 7.72 (dd, J=8.6, 1.9 Hz, 1H), 7.66 (d, J=8.7 Hz, 1H), 7.47-7.42 (m, 3H), 7.38 (d, J=8.6 Hz, 2H), 5.18 (t, J=7.3 Hz, 1H), 3.90 (s, 3H), 3.67-3.59 (m, 2H), 3.23 (s, 2H), 2.81-2.77 (m, 2H), 1.89 (t, J=10.6 Hz, 2H), 1.48 (d, J=7.0 Hz, 3H), 1.05 (d, J=6.3 Hz, 6H).

### Example 86: Preparation of Compound 86

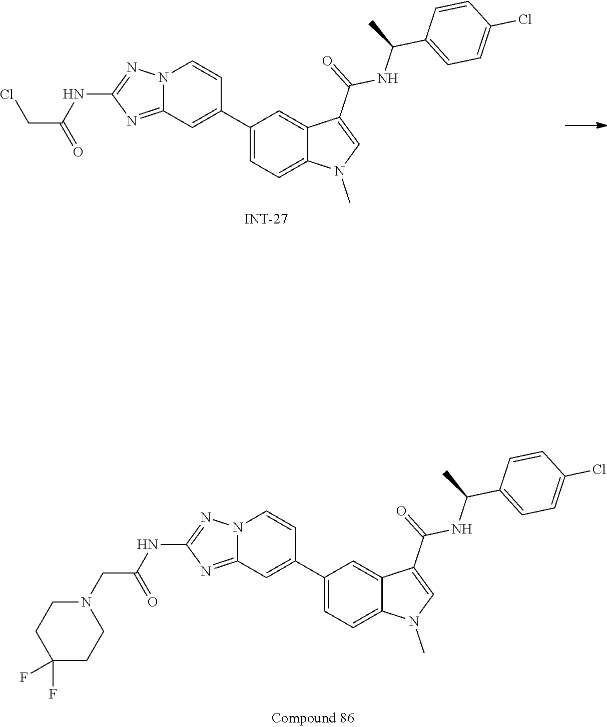 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CCC(F)(F)CC5)nc4c3)cc12)c1ccc(Cl)cc1]

### Preparation of Compound 86

Intermediate INT-27 (104 mg, 0.2 mmol) was dissolved in acetonitrile (1 mL); 4,4-difluoropiperidine (48 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 86 with a yield of 71%. MS (ESI, positive ion) m/z: 606.2 [M+H]⁺.

¹H NMR (400 MHz, DMSO-d₆) δ 8.89 (d, J=7.1 Hz, 1H), 8.55 (d, J=1.9 Hz, 1H), 8.42 (d, J=7.9 Hz, 1H), 8.24 (s, 1H), 7.90 (d, J=2.0 Hz, 1H), 7.74 (dd, J=8.7, 1.9 Hz, 1H), 7.68 (d, J=8.7 Hz, 1H), 7.49-7.44 (m, 3H), 7.41 (d, J=8.5 Hz, 2H), 5.20 (p, J=7.2 Hz, 1H), 3.92 (s, 3H), 3.39 (s, 2H), 2.72 (t, J=5.7 Hz, 4H), 2.03 (dt, J=14.6, 7.8 Hz, 4H), 1.50 (d, J=7.0 Hz, 3H).

### Example 87: Preparation of Compound 87

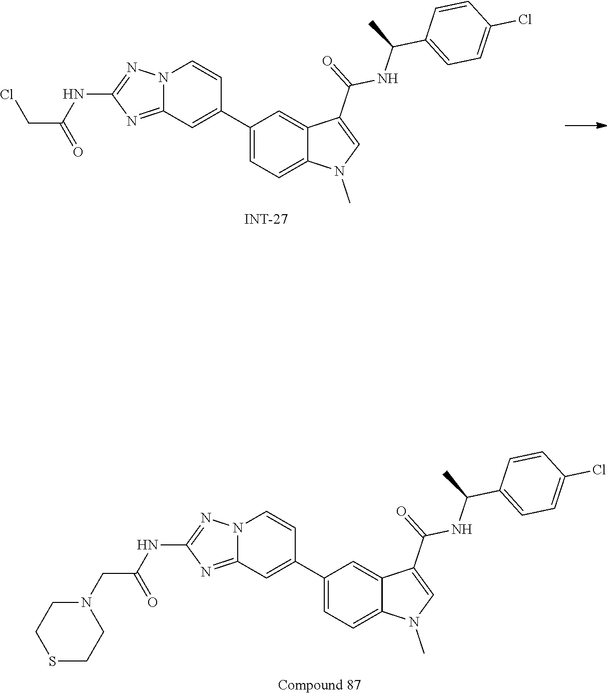 [SMILES: C[C@H](NC(=O)c1cn(C)c2ccc(-c3ccn4nc(NC(=O)CCl)nc4c3)cc12)c1ccc(Cl)cc1 ; "INT-27" ; C[C@H](NC(=O)c1cn(C)c2ccc(-c3ccn4nc(NC(=O)CN5CCSCC5)nc4c3)cc12)c1ccc(Cl)cc1]

### Preparation of Compound 87

Intermediate INT-27 (104 mg, 0.2 mmol) was dissolved in acetonitrile (1 mL); thiomorpholine (41 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 87 with a yield of 61%. MS (ESI, positive ion) m/z: 588.2 [M+H]⁺.

¹H NMR (400 MHz, DMSO-d₆) δ 10.47 (s, 1H), 8.88 (d, J=7.1 Hz, 1H), 8.55 (d, J=1.8 Hz, 1H), 8.39 (d, J=7.9 Hz, 1H), 8.24 (s, 1H), 7.90 (d, J=1.9 Hz, 1H), 7.74 (dd, J=8.6, 1.9 Hz, 1H), 7.68 (d, J=8.7 Hz, 1H), 7.49-7.45 (m, 3H), 7.42-7.39 (m, 2H), 5.21 (p, J=7.2 Hz, 1H), 3.92 (s, 3H), 3.31 (s, 2H), 2.84 (dd, J=6.4, 3.5 Hz, 4H), 2.70-2.66 (m, 4H), 1.50 (d, J=7.1 Hz, 3H).

### Example 88: Preparation of Compound 88

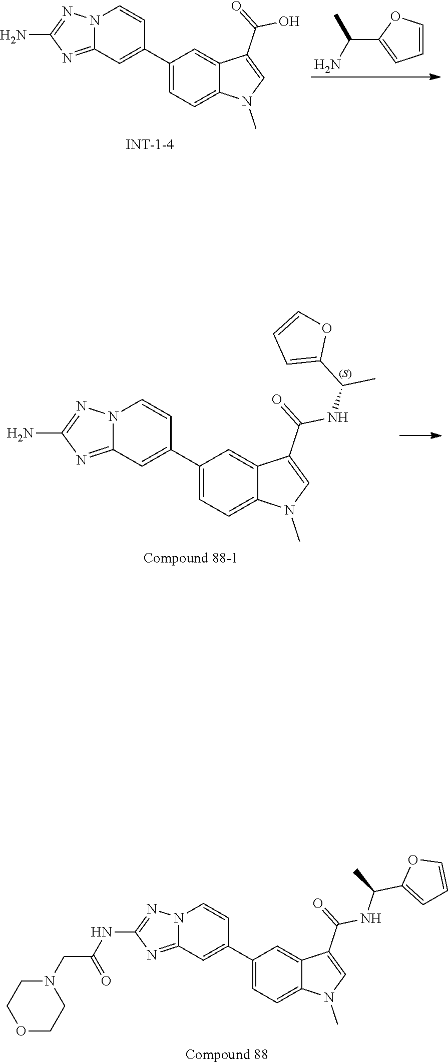

### Preparation of Compound 88-1

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(furan-2-yl)ethan-1-amine (133 mg, 1.2 mmol) was added, and the mixture was stirred for reaction at room temperature for 2 h; the reaction mixture was added dropwise into water and stirred for 0.5 h, filtered, and dried to obtain intermediate compound 88-1 with a yield of 76%. MS (ESI, positive ion) m/z: 401.3 [M+H]⁺.

### Preparation of Compound 88

Compound 88-1 (80 mg, 0.2 mmol) was dissolved in pyridine (1 mL), 2-(morpholin-4-yl)acetic acid (43 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 88 with a yield of 69%. MS (ESI, positive ion) m/z: 528.2 [M+H]⁺.

¹H NMR (400 MHz, DMSO-d₆) δ 10.52 (s, 1H), 8.90 (d, J=7.1 Hz, 1H), 8.61 (d, J=1.8 Hz, 1H), 8.31 (d, J=8.4 Hz, 1H), 8.19 (s, 1H), 7.92 (d, J=1.8 Hz, 1H), 7.75 (dd, J=8.6, 1.9 Hz, 1H), 7.67 (d, J=8.6 Hz, 1H), 7.60 (d, J=1.8 Hz, 1H), 7.50 (dd, J=7.2, 2.0 Hz, 1H), 6.42 (dd, J=3.3, 1.8 Hz, 1H), 6.30 (d, J=3.2 Hz, 1H), 5.34 (p, J=7.2 Hz, 1H), 3.90 (s, 3H), 3.65 (t, J=4.6 Hz, 4H), 3.28 (s, 2H), 2.57 (dd, J=5.6, 3.7 Hz, 4H), 1.52 (d, J=7.0 Hz, 3H).

### Example 89: Preparation of Compound 89

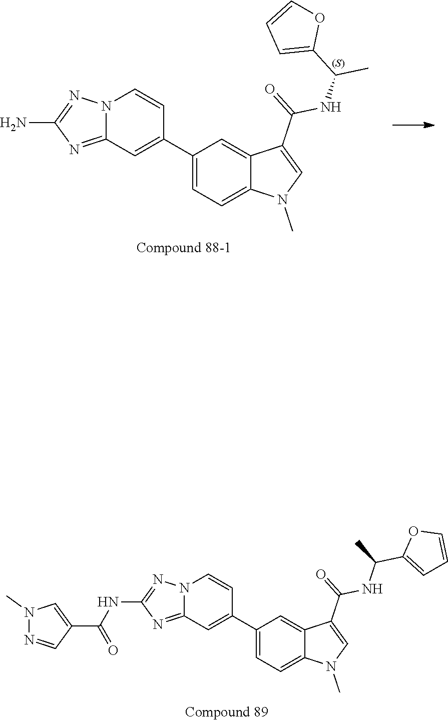

### Preparation of Compound 89

Compound 88-1 (80 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 89 with a yield of 62%. MS (ESI, positive ion) m/z: 509.2 [M+H]⁺.

¹H NMR (400 MHz, DMSO-d₆) δ 10.93 (s, 1H), 8.96-8.90 (m, 1H), 8.62 (d, J=1.9 Hz, 1H), 8.43 (s, 1H), 8.33 (d, J=8.4 Hz, 1H), 8.20 (s, 1H), 8.13 (s, 1H), 7.96-7.91 (m, 1H), 7.77 (dd, J=8.6, 1.9 Hz, 1H), 7.69 (d, J=8.7 Hz, 1H), 7.65-7.58 (m, 1H), 7.51 (dd, J=7.2, 2.0 Hz, 1H), 6.43 (dd, J=3.2, 1.8 Hz, 1H), 6.31 (dt, J=3.3, 1.0 Hz, 1H), 5.34 (p, J=7.1 Hz, 1H), 3.91 (d, J=5.9 Hz, 6H), 1.52 (d, J=7.0 Hz, 3H).

### Example 90: Preparation of Compound 90

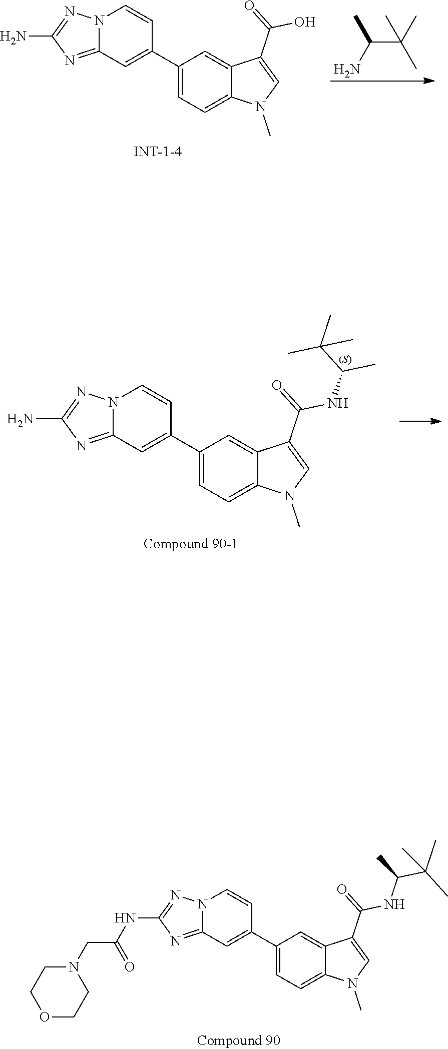

### Preparation of Compound 90-1

Intermediate INT-1-4 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(tert-butyl)ethan-1-amine (121 mg, 1.2 mmol) was added, and the mixture was stirred for reaction at room temperature for 2 h; the reaction mixture was added dropwise into water and stirred for 0.5 h, filtered, and dried to obtain intermediate compound 90-1 with a yield of 56%. MS (ESI, positive ion) m/z: 391.3 [M+H]⁺.

### Preparation of Compound 90

Compound 90-1 (78 mg, 0.2 mmol) was dissolved in pyridine (1 mL), 2-(morpholin-4-yl)acetic acid (43 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 90 with a yield of 59%. MS (ESI, positive ion) m/z: 518.2 [M+H]⁺.

¹H NMR (400 MHz, DMSO-d₆) δ 10.54 (s, 1H), 8.92-8.89 (m, 1H), 8.57 (d, J=1.8 Hz, 1H), 8.22 (s, 1H), 7.91 (dd, J=2.0, 0.9 Hz, 1H), 7.74 (dd, J=8.6, 1.9 Hz, 1H), 7.67 (d, J=8.6 Hz, 1H), 7.52-7.47 (m, 2H), 4.09-4.00 (m, 1H), 3.90 (s, 3H), 3.65 (t, J=4.6 Hz, 4H), 3.28 (s, 2H), 2.57 (t, J=4.7 Hz, 4H), 1.12 (d, J=6.9 Hz, 3H), 0.95 (s, 9H).

### Example 91: Preparation of Compound 91

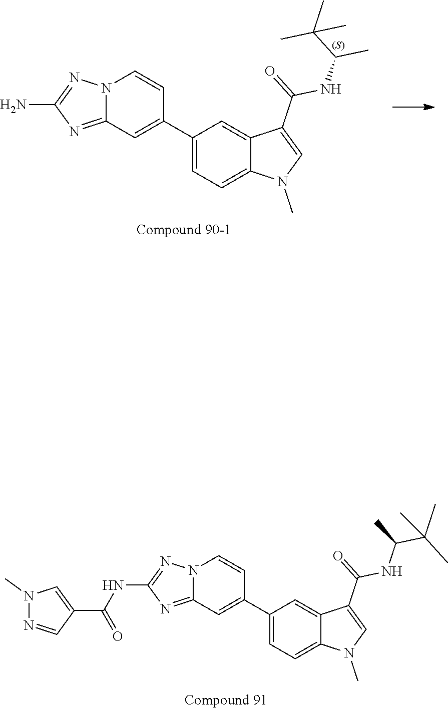

### Preparation of Compound 91

Compound 90-1 (78 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 91 with a yield of 58%. MS (ESI, positive ion) m/z: 499.2 [M+H]⁺.

¹H NMR (400 MHz, DMSO-d₆) δ 10.93 (s, 1H), 8.93 (d, J=7.1 Hz, 1H), 8.59 (d, J=1.8 Hz, 1H), 8.43 (s, 1H), 8.22 (s, 1H), 8.13 (s, 1H), 7.93 (t, J=1.3 Hz, 1H), 7.76 (dd, J=8.6, 1.9 Hz, 1H), 7.67 (d, J=8.6 Hz, 1H), 7.54-7.47 (m, 2H), 4.11-3.99 (m, 1H), 3.91 (d, J=4.1 Hz, 6H), 1.12 (d, J=6.9 Hz, 3H), 0.95 (s, 9H).

### Example 92: Preparation of Compound 92

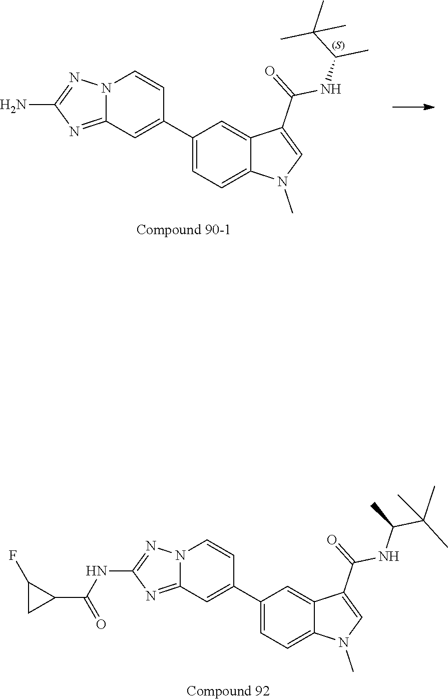

### Preparation of Compound 92

Compound 90-1 (78 mg, 0.2 mmol) was dissolved in pyridine (1 mL), 2-fluorocyclopropanecarboxylic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 92 with a yield of 58%. MS (ESI, positive ion) m/z: 477.2 [M+H]⁺.

¹H NMR (400 MHz, DMSO-d₆) δ 8.89 (d, J=7.1 Hz, 1H), 8.58 (d, J=1.8 Hz, 1H), 8.22 (s, 1H), 7.90 (d, J=2.2 Hz, 1H), 7.75 (dd, J=8.7, 1.9 Hz, 1H), 7.67 (d, J=8.7 Hz, 1H), 7.55-7.45 (m, 2H), 4.05 (dd, J=9.3, 6.9 Hz, 1H), 3.90 (s, 3H), 1.75-1.60 (m, 1H), 1.26-1.16 (m, 1H), 1.12 (d, J=6.9 Hz, 3H), 0.95 (s, 11H).

### Example 93: Preparation of Compound 93

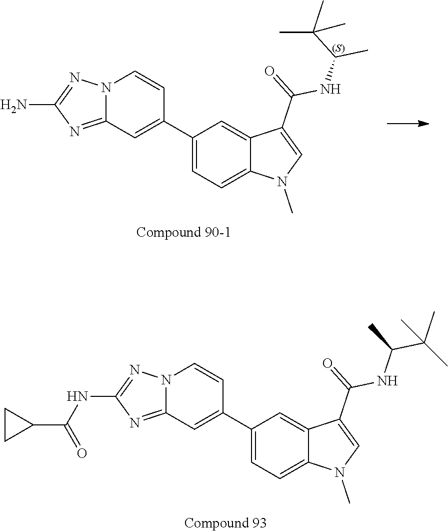

### Preparation of Compound 93

Compound 90-1 (78 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 93 with a yield of 58%. MS (ESI, positive ion) m/z: 459.2 [M+H]⁺.

¹H NMR (400 MHz, DMSO-d₆) δ 11.10 (s, 1H), 8.88 (d, J=7.0 Hz, 1H), 8.58 (d, J=1.8 Hz, 1H), 8.22 (s, 1H), 7.92-7.87 (m, 1H), 7.74 (dd, J=8.7, 1.9 Hz, 1H), 7.67 (d, J=8.6 Hz, 1H), 7.53-7.46 (m, 2H), 4.09-4.00 (m, 1H), 3.90 (s, 3H), 1.12 (d, J=6.9 Hz, 3H), 0.95 (s, 10H), 0.86 (d, J=6.1 Hz, 4H).

### Example 94: Preparation of Compound 94

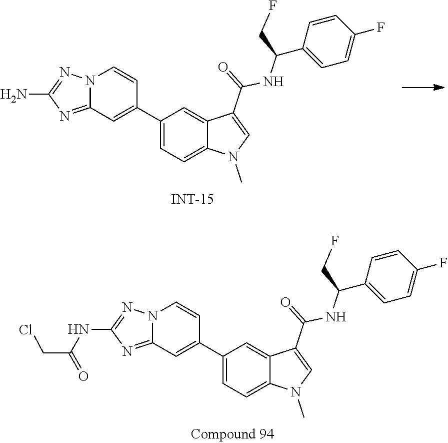

### Preparation of Compound 94

Intermediate INT-15 (44.6 g, 100 mmol) was dissolved in dichloromethane (1 L), chloroacetyl chloride (13.5 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Compound 94 with a yield of 73%. MS (ESI, positive ion) m/z: 523.2 [M+H]⁺.

¹H NMR (400 MHz, DMSO-d₆) δ 11.26 (s, 1H), 8.92 (d, J=7.1 Hz, 1H), 8.62 (d, J=8.3 Hz, 1H), 8.55 (d, J=1.8 Hz, 1H), 8.27 (s, 1H), 7.94 (d, J=1.9 Hz, 1H), 7.77 (dd, J=8.6, 1.9 Hz, 1H), 7.69 (d, J=8.7 Hz, 1H), 7.59-7.53 (m, 2H), 7.50 (dd, J=7.2, 2.0 Hz, 1H), 7.23 (t, J=8.9 Hz, 2H), 5.48 (dq, J=14.6, 7.2 Hz, 1H), 4.84-4.58 (m, 2H), 4.46 (s, 2H), 3.93 (s, 3H).

### Example 95: Preparation of Compound 95

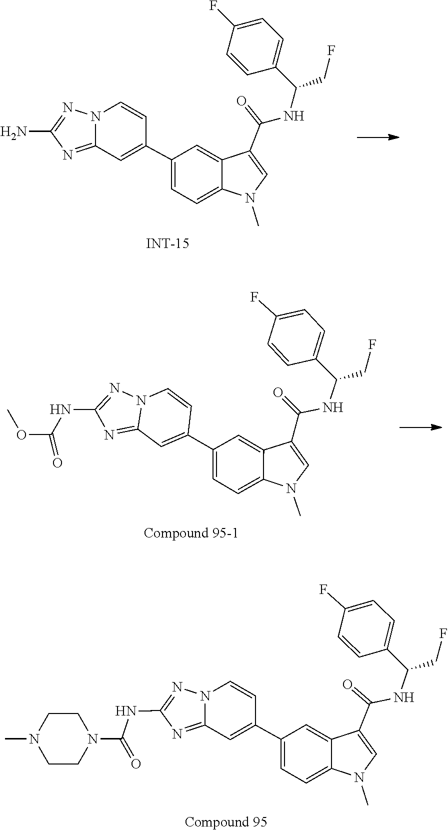 [SMILES: Cn1cc(C(=O)N[C@@H](CF)c2ccc(F)cc2)c2cc(-c3ccn4nc(N)nc4c3)ccc21 ; "INT-15" ; COC(=O)Nc1nc2cc(-c3ccc4c(c3)c(C(=O)N[C@@H](CF)c3ccc(F)cc3)cn4C)ccn2n1 ; CN1CCN(C(=O)Nc2nc3cc(-c4ccc5c(c4)c(C(=O)N[C@@H](CF)c4ccc(F)cc4)cn5C)ccn3n2)CC1]

### Preparation of Compound 95-1

Intermediate INT-15 (44.6 g, 100 mmol) was dissolved in dichloromethane (1 L), methyl chloroformate (11.2 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Compound 95-1 with a yield of 69%. MS (ESI, positive ion) m/z: 505.2 [M+H]⁺.

### Preparation of Compound 95

Compound 95-1 (100 mg, 0.2 mmol) was dissolved in acetonitrile (1 mL); N-methylpiperazine (40 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 95 with a yield of 61%. MS (ESI, positive ion) m/z: 573.2 [M+H]⁺.

¹H NMR (400 MHz, DMSO-d₆) δ 9.65 (s, 1H), 8.83 (d, J=7.1 Hz, 1H), 8.61 (d, J=8.3 Hz, 1H), 8.52 (d, J=1.8 Hz, 1H), 8.27 (s, 1H), 7.86 (dd, J=1.9, 0.9 Hz, 1H), 7.78 (dd, J=8.7, 1.9 Hz, 1H), 7.69 (d, J=8.6 Hz, 1H), 7.59-7.53 (m, 2H), 7.43 (dd, J=7.1, 2.0 Hz, 1H), 7.23 (t, J=8.9 Hz, 2H), 5.48 (dq, J=14.9, 7.2 Hz, 1H), 4.75 (dd, J=6.6, 3.3 Hz, 1H), 4.68-4.60 (m, 1H), 4.23 (s, 3H), 3.93 (s, 3H), 3.55-3.60 (m, 4H), 3.48 (t, J=4.8 Hz, 4H).

### Example 96: Preparation of Compound 96

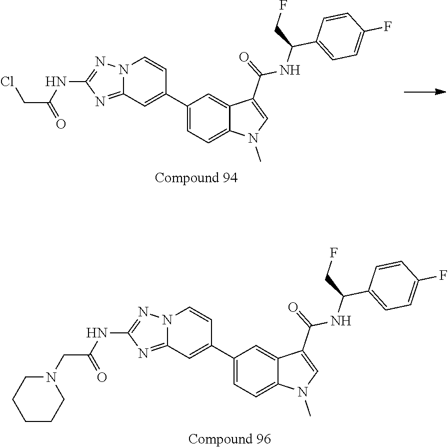 [SMILES: Cn1cc(C(=O)N[C@@H](CF)c2ccc(F)cc2)c2cc(-c3ccn4nc(NC(=O)CCl)nc4c3)ccc21 ; "Compound 94" ; Cn1cc(C(=O)N[C@@H](CF)c2ccc(F)cc2)c2cc(-c3ccn4nc(NC(=O)CN5CCCCC5)nc4c3)ccc21]

### Preparation of Compound 96

Intermediate Compound 96 (104 mg, 0.2 mmol) was dissolved in acetonitrile (1 mL); piperidine (34 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 96 with a yield of 58%. MS (ESI, positive ion) m/z: 572.2 [M+H]⁺.

¹H NMR (400 MHz, DMSO-d₆) δ 9.64 (s, 1H), 8.85 (d, J=7.1 Hz, 1H), 8.61 (d, J=8.3 Hz, 1H), 8.54 (d, J=1.8 Hz, 1H), 8.26 (s, 1H), 7.85 (dd, J=1.9, 0.9 Hz, 1H), 7.75 (dd, J=8.7, 1.9 Hz, 1H), 7.67 (d, J=8.6 Hz, 1H), 7.56-7.53 (m, 2H), 7.43 (dd, J=7.1, 2.0 Hz, 1H), 7.23 (t, J=8.9 Hz, 2H), 5.46 (dq, J=14.9, 7.2 Hz, 1H), 4.75 (dd, J=6.6, 3.3 Hz, 1H), 4.65-4.60 (m, 1H), 3.93 (s, 3H), 3.67 (m, 2H), 3.55-3.60 (m, 4H), 1.32 (m, 4H), 1.10 (m, 2H).

### Example 97: Preparation of Compound 97

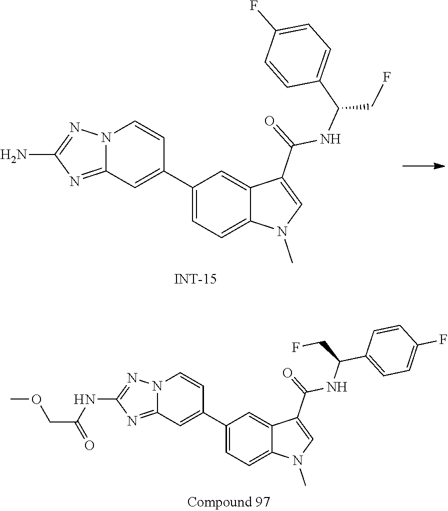 [SMILES: Cn1cc(C(=O)N[C@@H](CF)c2ccc(F)cc2)c2cc(-c3ccn4nc(N)nc4c3)ccc21 ; "INT-15" ; COCC(=O)Nc1nc2cc(-c3ccc4c(c3)c(C(=O)N[C@@H](CF)c3ccc(F)cc3)cn4C)ccn2n1]

### Preparation of Compound 97

INT-15 (89 mg, 0.2 mmol) was dissolved in pyridine (1 mL), 2-methoxyacetic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 97 with a yield of 68%. MS (ESI, positive ion) m/z: 519.2 [M+H]⁺.

¹H NMR (400 MHz, DMSO-d₆) δ 9.65 (s, 1H), 8.86 (d, J=7.1 Hz, 1H), 8.65 (d, J=8.3 Hz, 1H), 8.54 (d, J=1.8 Hz, 1H), 8.25 (s, 1H), 7.86 (dd, J=1.9, 0.9 Hz, 1H), 7.75 (dd, J=8.7, 1.9 Hz, 1H), 7.72 (d, J=8.6 Hz, 1H), 7.59-7.53 (m, 2H), 7.42 (dd, J=7.1, 2.0 Hz, 1H), 7.21 (t, J=8.9 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 3.12 (m, 2H), 2.87 (m, 2H).

### Example 98: Preparation of Compound 98

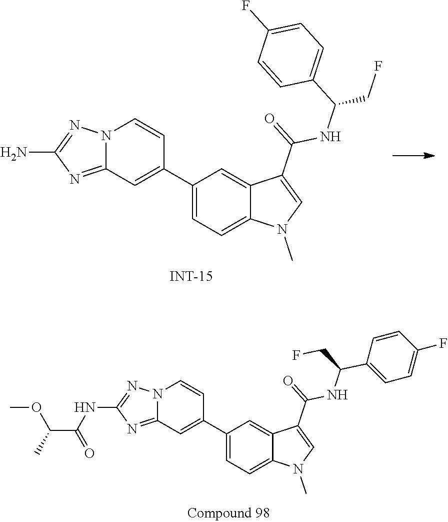 [SMILES: Cn1cc(C(=O)N[C@@H](CF)c2ccc(F)cc2)c2cc(-c3ccn4nc(N)nc4c3)ccc21 ; "INT-15" ; CO[C@@H](C)C(=O)Nc1nc2cc(-c3ccc4c(c3)c(C(=O)N[C@@H](CF)c3ccc(F)cc3)cn4C)ccn2n1]

### Preparation of Compound 98

INT-15 (89 mg, 0.2 mmol) was dissolved in pyridine (1 mL), (S)-2-methoxypropanoic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 98 with a yield of 61%. MS (ESI, positive ion) m/z: 533.2 [M+H]⁺.

¹H NMR (400 MHz, DMSO-d₆) δ 8.61 (t, J=8.3 Hz, 2H), 8.49 (d, J=1.8 Hz, 1H), 8.25 (s, 1H), 7.73-7.63 (m, 2H), 7.61 (d, J=2.0 Hz, 1H), 7.55 (dd, J=8.6, 5.6 Hz, 2H), 7.27-7.17 (m, 3H), 6.58 (t, J=6.1 Hz, 1H), 5.49 (dt, J=15.5, 7.0 Hz, 1H), 4.75 (d, J=7.7 Hz, 1H), 4.67-4.60 (m, 1H), 3.92 (s, 3H), 3.55 (q, J=6.1 Hz, 1H), 3.22 (dt, J=12.9, 6.1 Hz, 1H), 1.25 (s, 3H), 1.13 (d, J=6.1 Hz, 3H).

### Example 99: Preparation of Compound 99

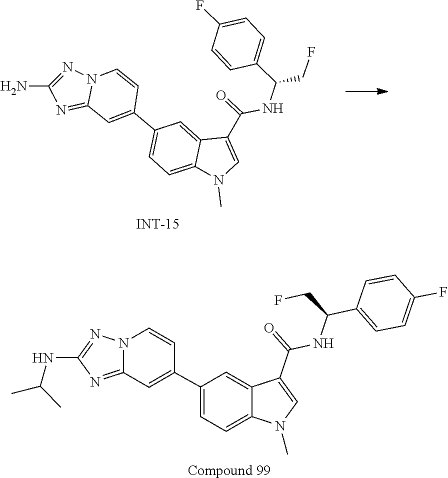 [SMILES: Cn1cc(C(=O)N[C@@H](CF)c2ccc(F)cc2)c2cc(-c3ccn4nc(N)nc4c3)ccc21 ; "INT-15" ; CC(C)Nc1nc2cc(-c3ccc4c(c3)c(C(=O)N[C@@H](CF)c3ccc(F)cc3)cn4C)ccn2n1 ; "Compound 99"]

### Preparation of Compound 99

INT-15 (89 mg, 0.2 mmol) was dissolved in THF (1 mL), acetone (0.2 mL) and tetraisopropyl titanate (0.1 mL) were added, and the mixture was heated to 80° C. and stirred for 2 h. After cooling to room temperature, sodium borohydride (23 mg, 0.6 mmol) was added. After 1 h, the reaction mixture was concentrated under reduced pressure and purified by C18 reverse phase silica gel column (acetonitrile/0.1% aqueous ammonium bicarbonate solution=60%) to obtain Compound 99 with a yield of 21%. MS (ESI, positive ion) m/z: 489.2 [M+H]⁺.

¹H NMR (400 MHz, DMSO-d₆) δ 9.64 (s, 1H), 8.85 (d, J=7.1 Hz, 1H), 8.64 (d, J=8.3 Hz, 1H), 8.53 (d, J=1.8 Hz, 1H), 8.27 (s, 1H), 7.86 (dd, J=1.9, 0.9 Hz, 1H), 7.75 (dd, J=8.7, 1.9 Hz, 1H), 7.67 (d, J=8.6 Hz, 1H), 7.61-7.53 (m, 2H), 7.45 (dd, J=7.1, 2.0 Hz, 1H), 7.22 (t, J=8.9 Hz, 2H), 4.75 (dd, J=6.6, 3.3 Hz, 1H), 3.96 (m, 1H), 3.76 (m, 2H), 3.50 (s, 3H), 1.21 (d, J=8.6 Hz, 6H).

### Example 100: Preparation of Compound 100

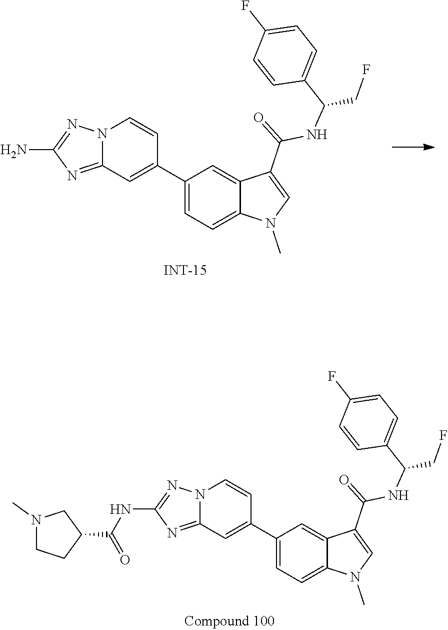 [SMILES: Cn1cc(C(=O)N[C@@H](CF)c2ccc(F)cc2)c2cc(-c3ccn4nc(N)nc4c3)ccc21 ; "INT-15" ; CN1CC[C@@H](C(=O)Nc2nc3cc(-c4ccc5c(c4)c(C(=O)N[C@@H](CF)c4ccc(F)cc4)cn5C)ccn3n2)C1]

### Preparation of Compound 100

INT-15 (89 mg, 0.2 mmol) was dissolved in pyridine (1 mL), (R)-1-methylpyrrolidine-3-carboxylic acid (39 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 100 with a yield of 78%. MS (ESI, positive ion) m/z: 558.2 [M+H]⁺.

¹H NMR (400 MHz, DMSO-d₆) δ 9.63 (s, 1H), 8.85 (d, J=7.1 Hz, 1H), 8.64 (d, J=8.3 Hz, 1H), 8.55 (d, J=1.8 Hz, 1H), 8.27 (s, 1H), 7.83 (dd, J=1.9, 0.9 Hz, 1H), 7.75 (dd, J=8.7, 1.9 Hz, 1H), 7.69 (d, J=8.6 Hz, 1H), 7.59-7.53 (m, 2H), 7.43 (dd, J=7.1, 2.0 Hz, 1H), 7.24 (t, J=8.9 Hz, 2H), 5.48 (dq, J=14.9, 7.2 Hz, 1H), 4.75 (dd, J=6.6, 3.3 Hz, 1H), 3.59 (m, 1H), 1.50 (d, J=7.1 Hz, 6H).

### Example 101: Preparation of Compound 101

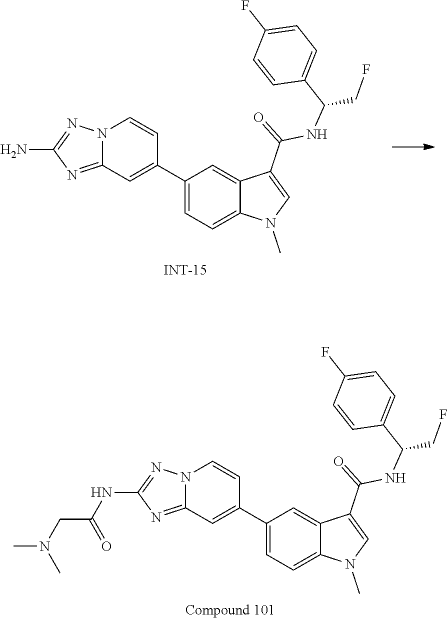 [SMILES: Cn1cc(C(=O)N[C@@H](CF)c2ccc(F)cc2)c2cc(-c3ccn4nc(N)nc4c3)ccc21 ; "INT-15" ; CN(C)CC(=O)Nc1nc2cc(-c3ccc4c(c3)c(C(=O)N[C@@H](CF)c3ccc(F)cc3)cn4C)ccn2n1]

### Preparation of Compound 101

INT-15 (89 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 101 with a yield of 78%. MS (ESI, positive ion) m/z: 532.2 [M+H]⁺.

¹H NMR (400 MHz, DMSO-d₆) δ 9.64 (s, 1H), 8.85 (d, J=7.1 Hz, 1H), 8.64 (d, J=8.3 Hz, 1H), 8.53 (d, J=1.8 Hz, 1H), 8.27 (s, 1H), 7.86 (dd, J=1.9, 0.9 Hz, 1H), 7.75 (dd, J=8.7, 1.9 Hz, 1H), 7.67 (d, J=8.6 Hz, 1H), 7.61-7.53 (m, 2H), 7.45 (dd, J=7.1, 2.0 Hz, 1H), 7.22 (t, J=8.9 Hz, 2H), 5.49 (dq, J=14.9, 7.2 Hz, 1H), 4.75 (dd, J=6.6, 3.3 Hz, 1H), 3.76 (m, 2H), 3.66 (m, 2H), 3.50 (s, 3H), 2.87 (m, 2H), 2.32 (m, 1H).

### Example 102: Preparation of Compound 102

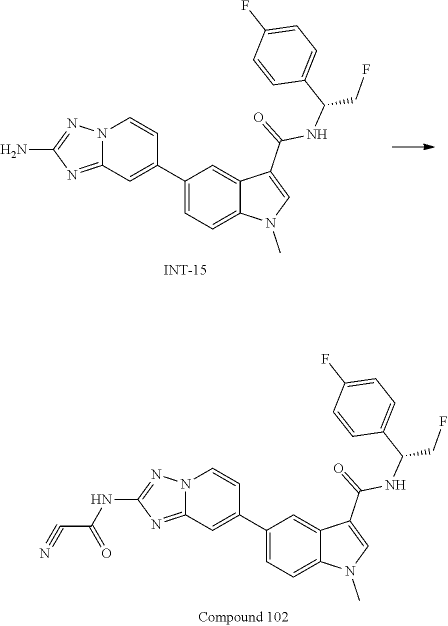 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NC(=O)C#N)nc4c3)ccc21]

### Preparation of Compound 102

INT-15 (89 mg, 0.2 mmol) was dissolved in pyridine (1 mL), cyanoacetic acid (29 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 102 with a yield of 78%. MS (ESI, positive ion) m/z: 500.2 [M+H]⁺.

¹H NMR (400 MHz, DMSO-d₆) δ 11.29 (s, 1H), 8.92 (d, J=7.1 Hz, 1H), 8.62 (d, J=8.2 Hz, 1H), 8.55 (d, J=1.8 Hz, 1H), 8.27 (s, 1H), 7.94 (d, J=1.8 Hz, 1H), 7.77 (dd, J=8.6, 1.9 Hz, 1H), 7.69 (d, J=8.7 Hz, 1H), 7.57-7.49 (m, 3H), 7.23 (t, J=8.8 Hz, 2H), 5.53-5.43 (m, 1H), 4.76 (d, J=8.0 Hz, 1H), 4.67-4.61 (m, 1H), 3.93 (s, 3H), 2.72-2.55 (m, 1H), 2.35 (s, 1H).

### Example 103: Preparation of Compound 103

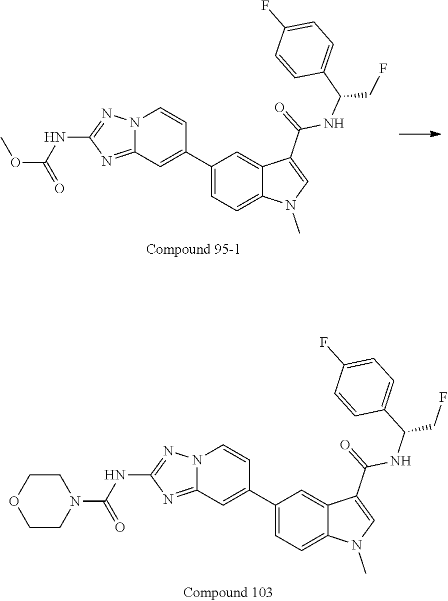 [SMILES: COC(=O)Nc1nc2cc(-c3ccc4c(c3)c(C(=O)N[C@@H](CF)c3ccc(F)cc3)cn4C)ccn2n1 ; "Compound 95-1" ; Cn1cc(C(=O)N[C@@H](CF)c2ccc(F)cc2)c2cc(-c3ccn4nc(NC(=O)N5CCOCC5)nc4c3)ccc21]

### Preparation of Compound 103

Compound 95-1 (100 mg, 0.2 mmol) was dissolved in acetonitrile (1 mL); morpholine (32 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 103 with a yield of 61%. MS (ESI, positive ion) m/z: 560.2 [M+H]⁺.

¹H NMR (400 MHz, DMSO-d₆) δ 9.63 (s, 1H), 8.84 (d, J=7.1 Hz, 1H), 8.69 (d, J=8.3 Hz, 1H), 8.54 (d, J=1.3 Hz, 1H), 8.26 (s, 1H), 7.86 (dd, J=1.9, 0.9 Hz, 1H), 7.75 (dd, J=8.7, 1.9 Hz, 1H), 7.72 (d, J=8.6 Hz, 1H), 7.59-7.53 (m, 2H), 7.43 (dd, J=7.1, 2.0 Hz, 1H), 7.21 (t, J=8.7 Hz, 2H), 5.49 (dq, J=14.9, 7.2 Hz, 1H), 4.76 (dd, J=6.6, 3.3 Hz, 1H), 4.68-4.60 (m, 1H), 3.93 (s, 3H), 3.55 (dd, J=7.6, 3.8 Hz, 4H), 3.31 (dd, J=7.4, 3.7 Hz, 4H).

### Example 104: Preparation of Compound 104

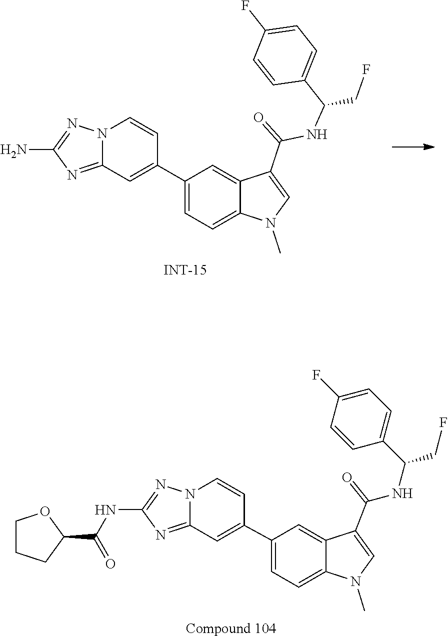 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NC(=O)[C@H]5CCCO5)nc4c3)ccc21 ; "Compound 104"]

### Preparation of Compound 104

INT-15 (89 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 104 with a yield of 71%. MS (ESI, positive ion) m/z: 545.2 [M+H]⁺.

¹H NMR (400 MHz, DMSO-d₆) δ 9.63 (s, 1H), 8.84 (d, J=7.1 Hz, 1H), 8.69 (d, J=8.3 Hz, 1H), 8.54 (d, J=1.3 Hz, 1H), 8.26 (s, 1H), 7.86 (dd, J=1.9, 0.9 Hz, 1H), 7.75 (dd, J=8.7, 1.9 Hz, 1H), 7.72 (d, J=8.6 Hz, 1H), 7.59-7.53 (m, 2H), 7.43 (dd, J=7.1, 2.0 Hz, 1H), 7.21 (t, J=8.7 Hz, 2H), 5.49 (dq, J=14.9, 7.2 Hz, 1H), 4.76 (dd, J=6.6, 3.3 Hz, 1H), 4.68-4.60 (m, 1H), 3.93 (s, 3H), 3.67 (m, 2H), 3.53 (m, 1H), 1.88 (m, 4H).

### Example 105: Preparation of Compound 105

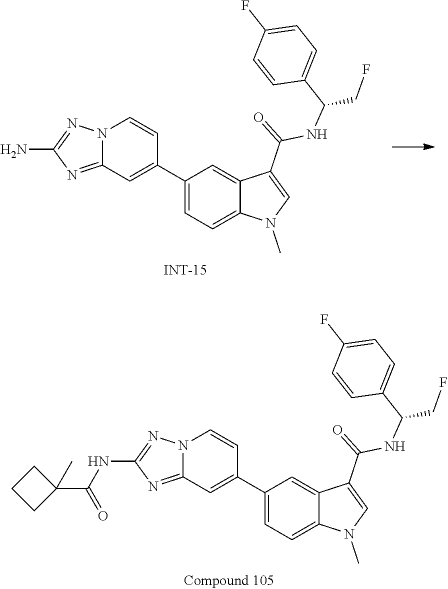 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NC(=O)C5(C)CCC5)nc4c3)ccc21]

### Preparation of Compound 105

INT-15 (89 mg, 0.2 mmol) was dissolved in pyridine (1 mL), 1-methylcyclobutanecarboxylic acid (34 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 105 with a yield of 71%. MS (ESI, positive ion) m/z: 543.2 [M+H]⁺.

¹H NMR (400 MHz, DMSO-d₆) δ 9.62 (s, 1H), 8.83 (d, J=7.1 Hz, 1H), 8.62 (d, J=8.3 Hz, 1H), 8.54 (d, J=1.8 Hz, 1H), 8.27 (s, 1H), 7.85 (dd, J=1.9, 0.9 Hz, 1H), 7.75 (dd, J=8.7, 1.9 Hz, 1H), 7.69 (d, J=8.6 Hz, 1H), 7.59-7.53 (m, 2H), 7.43 (dd, J=7.1, 2.0 Hz, 1H), 7.23 (t, J=8.9 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 2.78 (m, 4H), 1.23 (s, 3H).

### Example 106: Preparation of Compound 106

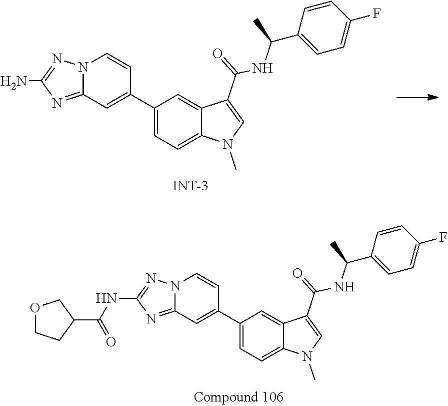

### Preparation of Compound 106

Intermediate INT-3 (85 mg, 0.2 mmol) was dissolved in pyridine (1 mL); 3-tetrahydrofuroic acid (30 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 106 with a yield of 55%. MS (ESI, positive ion) m/z: 527.2 [M+H]⁺.

¹H NMR (400 MHz, DMSO-d₆) δ 10.44 (s, 1H), 8.79 (d, J=7.4 Hz, 1H), 8.56 (d, J=1.4 Hz, 1H), 8.34 (d, J=8.1 Hz, 1H), 8.22 (s, 1H), 7.92 (d, J=1.9 Hz, 1H), 7.72 (dd, J=8.6, 1.8 Hz, 1H), 7.61 (d, J=8.7 Hz, 1H), 7.43 (ddt, J=5.8, 4.1, 1.7 Hz, 3H), 7.24-7.12 (m, 2H), 5.22 (p, J=7.2 Hz, 1H), 3.91 (s, 3H), 3.27 (s, 2H), 2.56 (dd, J=5.4, 3.6 Hz, 4H), 2.1 (m, 2H), 1.87 (m, 1H), 1.50 (d, J=7.0 Hz, 3H).

### Example 107: Preparation of Compound 107

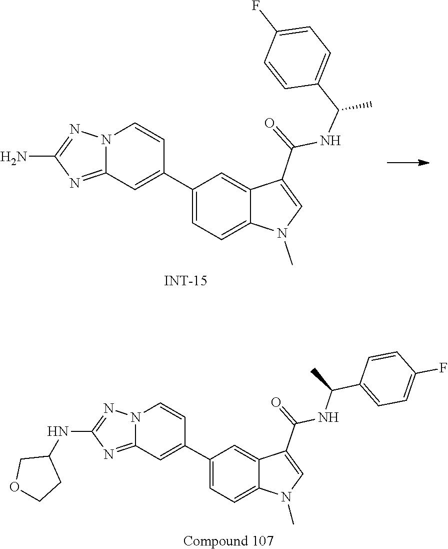 [SMILES: C[C@H](NC(=O)c1cn(C)c2ccc(-c3ccn4nc(N)nc4c3)cc12)c1ccc(F)cc1 ; "INT-15" ; C[C@H](NC(=O)c1cn(C)c2ccc(-c3ccn4nc(NC5CCOC5)nc4c3)cc12)c1ccc(F)cc1 ; "Compound 107"]

### Preparation of Compound 107

INT-15 (89 mg, 0.2 mmol) was dissolved in THF (1 mL), dihydro-3(2H)-furanone (0.2 mL) and tetraisopropyl titanate (0.1 mL) were added, and the mixture was heated to 80° C. and stirred for 2 h. After cooling to room temperature, sodium borohydride (23 mg, 0.6 mmol) was added. After 1 h, the reaction mixture was concentrated under reduced pressure and purified by C18 reverse phase silica gel column (acetonitrile/0.1% aqueous ammonium bicarbonate solution=60%) to obtain Compound 107 with a yield of 18%. MS (ESI, positive ion) m/z: 499.2 [M+H]⁺.

¹H NMR (400 MHz, DMSO-d₆) δ 10.56 (s, 1H), 8.87 (d, J=7.1 Hz, 1H), 8.53 (d, J=1.9 Hz, 1H), 8.38 (d, J=8.1 Hz, 1H), 8.22 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.23-7.11 (m, 2H), 5.24 (p, J=7.2 Hz, 1H), 3.92 (s, 3H), 3.76 (m, 1H), 2.56 (dd, J=5.4, 3.4 Hz, 4H), 2.15 (m, 2H), 1.50 (d, J=7.0 Hz, 3H).

### Example 108: Preparation of Compound 108

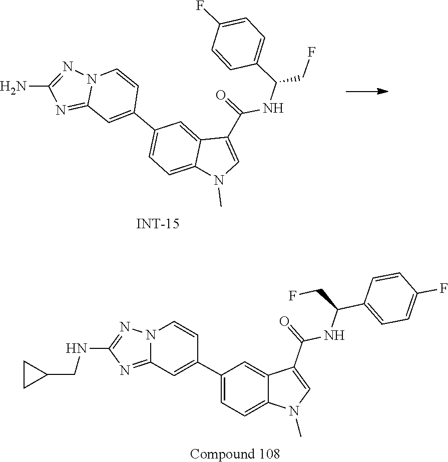 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NCC5CC5)nc4c3)ccc21 ; "Compound 108"]

### Preparation of Compound 108

INT-15 (89 mg, 0.2 mmol) was dissolved in THF (1 mL), cyclopropanecarbaldehyde (0.2 mL) and tetraisopropyl titanate (0.1 mL) were added, and the mixture was heated to 80° C. and stirred for 2 h. After cooling to room temperature, sodium borohydride (23 mg, 0.6 mmol) was added. After 1 h, the reaction mixture was concentrated under reduced pressure and purified by C18 reverse phase silica gel column (acetonitrile/0.1% aqueous ammonium bicarbonate solution=60%) to obtain Compound 108 with a yield of 23%. MS (ESI, positive ion) m/z: 501.2 [M+H]⁺.

¹H NMR (400 MHz, DMSO-d₆) δ 9.64 (s, 1H), 8.84 (d, J=7.1 Hz, 1H), 8.65 (d, J=8.2 Hz, 1H), 8.55 (d, J=1.9 Hz, 1H), 8.28 (s, 1H), 7.83 (dd, J=1.9, 0.9 Hz, 1H), 7.75 (dd, J=8.6, 1.9 Hz, 1H), 7.71 (d, J=8.6 Hz, 1H), 7.62-7.53 (m, 2H), 7.46 (dd, J=7.1, 2.0 Hz, 1H), 7.23 (t, J=8.8 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 3.23 (m, 2H), 1.10 (m, 5H)

### Example 109: Preparation of Compound 109

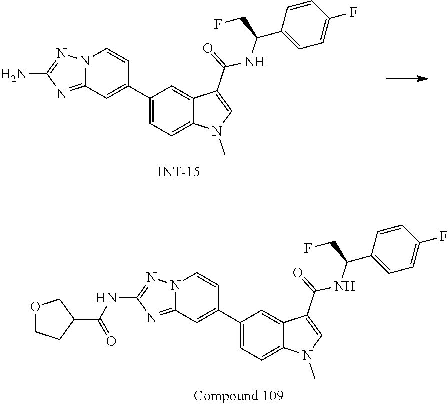 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NC(=O)C5CCOC5)nc4c3)ccc21]

### Preparation of Compound 109

Intermediate INT-15 (88 mg, 0.2 mmol) was dissolved in pyridine (1 mL); 3-tetrahydrofuroic acid (30 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 109 with a yield of 65%. MS (ESI, positive ion) m/z: 545.2 [M+H]⁺.

¹H NMR (400 MHz, DMSO-d₆) δ 9.63 (s, 1H), 8.84 (d, J=7.1 Hz, 1H), 8.65 (d, J=8.2 Hz, 1H), 8.57 (d, J=1.9 Hz, 1H), 8.29 (s, 1H), 7.84 (dd, J=1.9, 0.9 Hz, 1H), 7.76 (dd, J=8.6, 1.9 Hz, 1H), 7.73 (d, J=8.6 Hz, 1H), 7.63-7.53 (m, 2H), 7.44 (dd, J=7.1, 2.0 Hz, 1H), 7.23 (t, J=8.8 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 2.92 (m, 4H), 1.67 (m, 2H), 1.32 (m, 1H).

### Example 110: Preparation of Compound 110

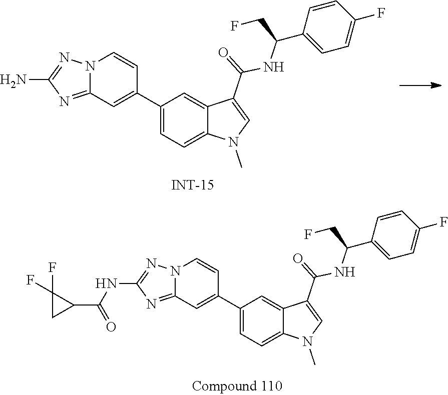 [SMILES: Cn1cc(C(=O)N[C@@H](CF)c2ccc(F)cc2)c2cc(-c3ccn4nc(N)nc4c3)ccc21 ; "INT-15" ; Cn1cc(C(=O)N[C@@H](CF)c2ccc(F)cc2)c2cc(-c3ccn4nc(NC(=O)C5CC5(F)F)nc4c3)ccc21]

### Preparation of Compound 110

Intermediate INT-15 (88 mg, 0.2 mmol) was dissolved in pyridine (1 mL); 2,2-difluorocyclopropanecarboxylic acid (36 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 110 with a yield of 65%. MS (ESI, positive ion) m/z: 551.2 [M+H]⁺.

¹H NMR (400 MHz, DMSO-d₆) δ 9.64 (s, 1H), 8.83 (d, J=7.2 Hz, 1H), 8.64 (d, J=8.3 Hz, 1H), 8.55 (d, J=1.9 Hz, 1H), 8.30 (s, 1H), 7.83 (dd, J=2.2, 0.9 Hz, 1H), 7.73 (dd, J=8.6, 1.9 Hz, 1H), 7.71 (d, J=8.4 Hz, 1H), 7.63-7.53 (m, 2H), 7.44 (dd, J=7.1, 2.0 Hz, 1H), 7.23 (t, J=8.8 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 2.02 (m, 2H), 1.86 (m, 1H).

### Example 111: Preparation of Compound 111

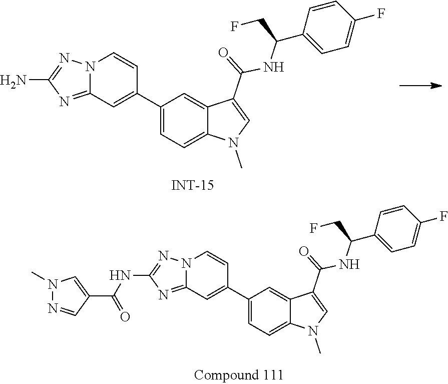

### Preparation of Compound 111

Intermediate INT-15 (88 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column chromatography (acetonitrile/0.1% aqueous ammonium bicarbonate solution=60%) to obtain Compound 111 with a yield of 45%. MS (ESI, positive ion) m/z: 555.2 [M+H]⁺.

¹H NMR (400 MHz, DMSO-d₆) δ 9.65 (s, 1H), 8.81 (d, J=7.2 Hz, 1H), 8.64 (d, J=8.3 Hz, 1H), 8.61 (d, J=1.9 Hz, 1H), 8.32 (s, 1H), 8.16 (s, 1H), 7.95 (s, 1H), 7.83 (dd, J=2.2, 0.9 Hz, 1H), 7.73 (dd, J=8.6, 1.9 Hz, 1H), 7.71 (d, J=8.4 Hz, 1H), 7.63-7.53 (m, 2H), 7.44 (dd, J=7.1, 2.0 Hz, 1H), 7.23 (t, J=8.8 Hz, 2H), 5.48 (dq, J=14.9, 7.2 Hz, 1H), 4.75 (dd, J=6.6, 3.3 Hz, 1H), 4.68-4.60 (m, 1H), 3.95 (s, 6H).

### Example 112: Preparation of Compound 112

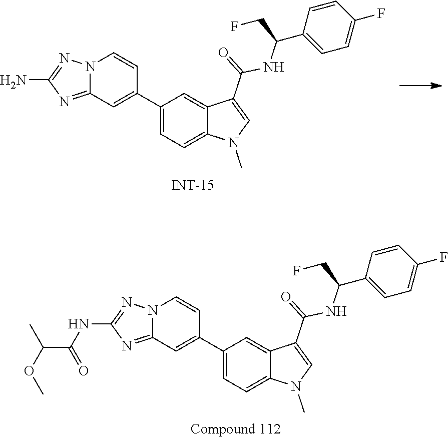

### Preparation of Compound 112

Intermediate INT-15 (88 mg, 0.2 mmol) was dissolved in pyridine (1 mL); 2-methoxypropionic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column chromatography (acetonitrile/0.1% aqueous ammonium bicarbonate solution=60%) to obtain Compound 112 with a yield of 45%. MS (ESI, positive ion) m/z: 533.2 [M+H]⁺.

¹H NMR (400 MHz, DMSO-d₆) δ 9.65 (s, 1H), 8.82 (d, J=7.3 Hz, 1H), 8.63 (d, J=8.5 Hz, 1H), 8.52 (d, J=1.9 Hz, 1H), 8.30 (s, 1H), 7.83 (dd, J=2.2, 0.9 Hz, 1H), 7.73 (dd, J=8.5, 1.9 Hz, 1H), 7.72 (d, J=8.2 Hz, 1H), 7.63-7.53 (m, 2H), 7.44 (dd, J=7.2, 2.0 Hz, 1H), 7.22 (t, J=8.8 Hz, 2H), 5.48 (dq, J=14.9, 7.2 Hz, 1H), 4.75 (dd, J=6.6, 3.3 Hz, 1H), 4.68-4.60 (m, 1H), 3.93 (s, 3H), 2.64 (s, 3H), 2.58 (m, 1H), 1.52 (d, J=2.8 Hz, 3H).

### Example 113: Preparation of Compound 113

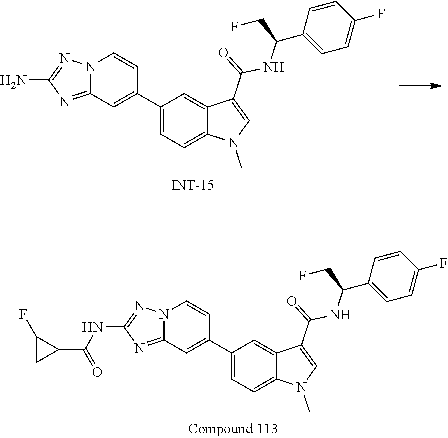

### Preparation of Compound 113

Intermediate INT-15 (88 mg, 0.2 mmol) was dissolved in pyridine (1 mL); 2-fluorocyclopropanecarboxylic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 113 with a yield of 55%. MS (ESI, positive ion) m/z: 533.2 [M+H]⁺.

¹H NMR (400 MHz, DMSO-d₆) δ 10.56 (s, 1H), 8.91 (d, J=7.2 Hz, 1H), 8.57 (d, J=1.6 Hz, 1H), 8.39 (d, J=8.2 Hz, 1H), 8.22 (s, 1H), 7.92 (d, J=1.9 Hz, 1H), 7.75 (dd, J=8.4, 1.6 Hz, 1H), 7.65 (d, J=8.7 Hz, 1H), 7.44 (ddt, J=5.8, 4.1, 1.7 Hz, 3H), 7.21-7.12 (m, 2H), 5.23 (p, J=7.2 Hz, 1H), 3.91 (s, 3H), 2.67 (m, 4H), 1.50 (d, J=7.0 Hz, 3H), 1.20 (s, 3H).

### Example 114: Preparation of Compound 114

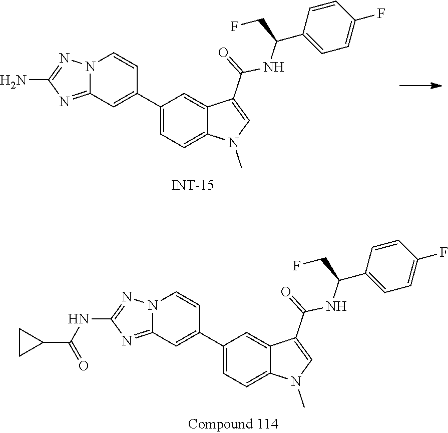

### Preparation of Compound 114

Intermediate INT-15 (88 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 114 with a yield of 55%. MS (ESI, positive ion) m/z: 515.2 [M+H]⁺.

¹H NMR (400 MHz, DMSO-d₆) δ 11.11 (s, 1H), 8.86 (d, J=7.1 Hz, 1H), 8.67 (d, J=8.3 Hz, 1H), 8.55 (d, J=1.8 Hz, 1H), 8.30 (s, 1H), 7.90 (d, J=2.0 Hz, 1H), 7.76 (dd, J=8.6, 1.9 Hz, 1H), 7.69 (d, J=8.7 Hz, 1H), 7.45 (ddd, J=14.3, 6.8, 1.8 Hz, 2H), 7.40-7.33 (m, 2H), 7.20-7.11 (m, 1H), 5.51 (dq, J=14.5, 6.6 Hz, 1H), 4.78 (d, J=6.5 Hz, 1H), 4.66 (d, J=6.5 Hz, 1H), 3.94 (s, 3H), 2.15-1.97 (m, 1H), 1.25 (d, J=2.9 Hz, 4H).

### Example 115: Preparation of Compound 115

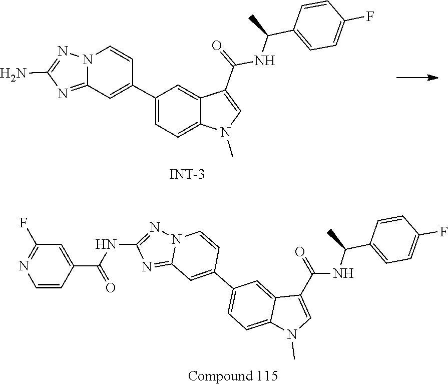 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)c5ccnc(F)c5)nc4c3)cc12)c1ccc(F)cc1]

### Preparation of Compound 115

Intermediate INT-3 (83 mg, 0.2 mmol) was dissolved in pyridine (1 mL); cyclopropanecarboxylic acid (39 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 115 with a yield of 55%. MS (ESI, positive ion) m/z: 552.2 [M+H]⁺.

¹H NMR (400 MHz, DMSO-d₆) δ 11.68 (s, 1H), 8.96 (dd, J=7.1, 0.8 Hz, 1H), 8.58 (d, J=1.8 Hz, 1H), 8.47 (d, J=5.2 Hz, 1H), 8.38 (d, J=8.0 Hz, 1H), 8.24 (s, 1H), 7.97 (dd, J=2.0, 0.9 Hz, 1H), 7.90 (dt, J=5.2, 1.7 Hz, 1H), 7.80-7.71 (m, 2H), 7.68 (d, J=8.6 Hz, 1H), 7.54 (dd, J=7.2, 2.0 Hz, 1H), 7.50-7.42 (m, 2H), 7.22-7.12 (m, 2H), 5.23 (p, J=7.1 Hz, 1H), 3.92 (s, 3H), 1.51 (d, J=7.1 Hz, 3H).

### Example 116: Preparation of Compound 116

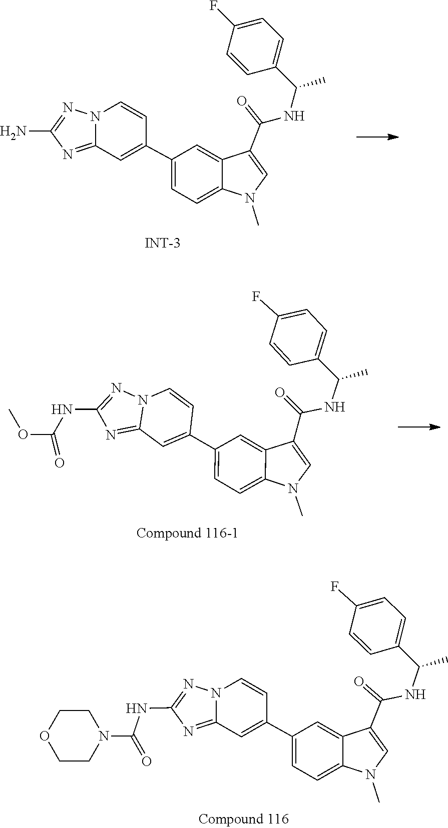

### Preparation of Compound 116-1

Intermediate INT-3 (42.8 g, 100 mmol) was dissolved in dichloromethane (1 L), methyl chloroformate (11.2 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Compound 116-1 with a yield of 65%. MS (ESI, positive ion) m/z: 487.2 [M+H]⁺.

### Preparation of Compound 116

Compound 116-1 (97 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 116 with a yield of 58%. MS (ESI, positive ion) m/z: 542.2 [M+H]⁺.

¹H NMR (400 MHz, DMSO-d₆) δ 9.60 (s, 1H), 8.85-8.79 (m, 1H), 8.60-8.48 (m, 1H), 8.37 (d, J=7.9 Hz, 1H), 8.23 (s, 1H), 7.84 (dd, J=2.1, 0.8 Hz, 1H), 7.74 (dd, J=8.6, 1.9 Hz, 1H), 7.67 (d, J=8.6 Hz, 1H), 7.49-7.45 (m, 2H), 7.43 (dd, J=7.1, 2.0 Hz, 1H), 7.17 (t, J=8.9 Hz, 2H), 5.21 (q, J=7.2 Hz, 1H), 3.91 (s, 3H), 3.66-3.58 (m, 4H), 3.48 (t, J=4.8 Hz, 4H), 1.50 (d, J=7.1 Hz, 3H).

### Example 117: Preparation of Compound 117

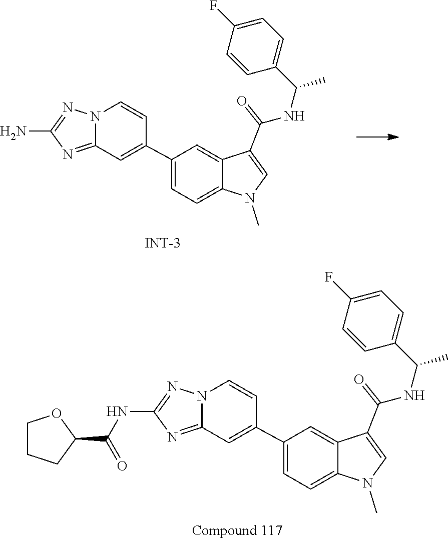 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(NC(=O)[C@H]5CCCO5)nc4c3)cc12)c1ccc(F)cc1 ; "Compound 117"]

### Preparation of Compound 117

INT-3 (83 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 117 with a yield of 69%. MS (ESI, positive ion) m/z: 527.2 [M+H]⁺.

¹H NMR (400 MHz, DMSO-d₆) δ 10.47 (s, 1H), 8.93-8.86 (m, 1H), 8.56 (d, J=1.9 Hz, 1H), 8.37 (d, J=8.0 Hz, 1H), 8.23 (s, 1H), 7.94-7.89 (m, 1H), 7.75 (dd, J=8.7, 1.9 Hz, 1H), 7.68 (d, J=8.6 Hz, 1H), 7.57-7.43 (m, 3H), 7.22-7.12 (m, 2H), 5.23 (p, J=7.1 Hz, 1H), 4.54 (s, 1H), 4.02 (q, J=6.9 Hz, 1H), 3.92 (s, 3H), 3.85 (ddd, J=7.9, 7.0, 5.9 Hz, 1H), 2.31-2.18 (m, 1H), 2.06-1.95 (m, 1H), 1.95-1.81 (m, 2H), 1.51 (d, J=7.1 Hz, 3H).

### Example 118: Preparation of Compound 118

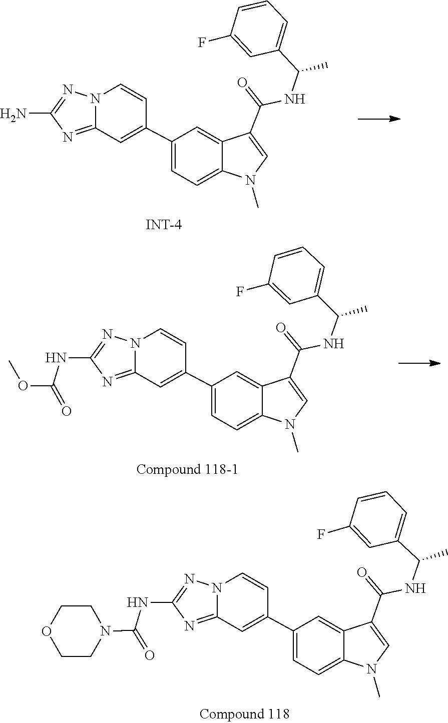

### Preparation of Compound 118-1

Intermediate INT-4 (42.8 g, 100 mmol) was dissolved in dichloromethane (1 L), methyl chloroformate (11.2 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Compound 118-1 with a yield of 65%. MS (ESI, positive ion) m/z: 487.2 [M+H]⁺.

### Preparation of Compound 118

Compound 118-1 (97 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 118 with a yield of 58%. MS (ESI, positive ion) m/z: 542.2 [M+H]⁺.

¹H NMR (400 MHz, DMSO-d₆) δ 10.45 (s, 1H), 8.85 (d, J=7.2 Hz, 1H), 8.53 (s, 1H), 8.42 (d, J=8.0 Hz, 1H), 8.24 (s, 1H), 7.93 (s, 1H), 7.76 (d, J=8.6 Hz, 1H), 7.69 (d, J=8.7 Hz, 1H), 7.48 (d, J=7.2 Hz, 1H), 7.42 (q, J=7.5 Hz, 1H), 7.27 (t, J=9.0 Hz, 2H), 7.11-7.02 (m, 1H), 5.25 (p, J=7.2 Hz, 1H), 3.92 (s, 3H), 3.50 (m, 4H), 2.67 (m, 4H), 1.52 (d, J=7.1 Hz, 3H).

### Example 119: Preparation of Compound 119

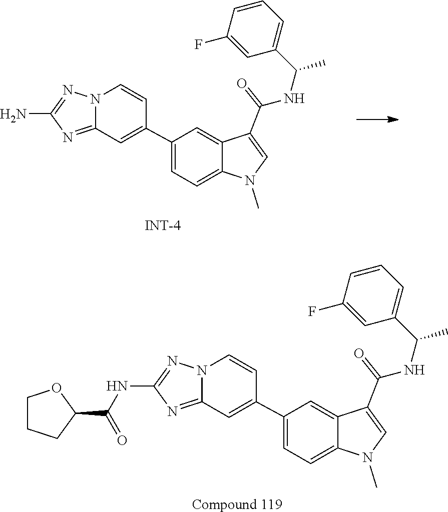 [SMILES: C[C@H](NC(=O)c1cn(C)c2ccc(-c3ccn4nc(N)nc4c3)cc12)c1cccc(F)c1 ; "INT-4" ; C[C@H](NC(=O)c1cn(C)c2ccc(-c3ccn4nc(NC(=O)[C@H]5CCCO5)nc4c3)cc12)c1cccc(F)c1 ; "Compound 119"]

### Preparation of Compound 119

INT-4 (83 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 119 with a yield of 69%. MS (ESI, positive ion) m/z: 527.2 [M+H]⁺.

¹H NMR (400 MHz, DMSO-d₆) δ 10.47 (s, 1H), 8.89 (d, J=7.2 Hz, 1H), 8.56 (s, 1H), 8.41 (d, J=8.0 Hz, 1H), 8.25 (s, 1H), 7.91 (s, 1H), 7.75 (d, J=8.6 Hz, 1H), 7.68 (d, J=8.7 Hz, 1H), 7.49 (d, J=7.2 Hz, 1H), 7.40 (q, J=7.5 Hz, 1H), 7.27 (t, J=9.0 Hz, 2H), 7.11-7.02 (m, 1H), 5.25 (p, J=7.2 Hz, 1H), 4.01 (t, J=7.1 Hz, 1H), 3.92 (s, 3H), 3.82 (dt, J=14.0, 7.1 Hz, 2H), 2.22 (ddq, J=26.5, 12.3, 7.3, 6.4 Hz, 2H), 1.93-1.84 (m, 3H), 1.52 (d, J=7.1 Hz, 3H).

### Example 120: Preparation of Compound 120

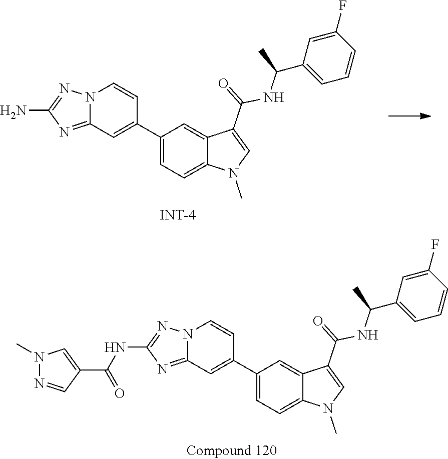 [SMILES: C[C@H](NC(=O)c1cn(C)c2ccc(-c3ccn4nc(N)nc4c3)cc12)c1cccc(F)c1 ; "INT-4" ; C[C@H](NC(=O)c1cn(C)c2ccc(-c3ccn4nc(NC(=O)c5cnn(C)c5)nc4c3)cc12)c1cccc(F)c1]

### Preparation of Compound 120

Intermediate INT-4 (83 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 120 with a yield of 45%. MS (ESI, positive ion) m/z: 537.2 [M+H]⁺.

¹H NMR (400 MHz, DMSO-d₆) δ 10.47 (s, 1H), 8.91 (dd, J=7.2, 0.8 Hz, 1H), 8.65 (d, J=1.9 Hz, 1H), 8.38 (d, J=8.2 Hz, 1H), 8.18 (s, 1H), 7.95 (dd, J=1.9, 0.9 Hz, 1H), 7.76 (dd, J=8.7, 1.9 Hz, 1H), 7.69 (d, J=8.6 Hz, 1H), 7.53 (dd, J=7.2, 2.0 Hz, 1H), 7.32 (dd, J=8.1, 5.4 Hz, 1H), 7.06 (td, J=8.6, 7.6, 1.7 Hz, 2H), 5.60 (q, J=8.1 Hz, 1H), 4.54 (s, 1H), 4.07-3.94 (m, 1H), 3.90 (s, 3H), 3.88-3.82 (m, 1H), 3.05-2.94 (m, 1H), 2.85 (dt, J=16.1, 8.5 Hz, 1H), 2.56 (dd, J=7.9, 3.1 Hz, 1H), 2.32-2.16 (m, 1H), 2.09-1.88 (m, 4H).

### Example 121: Preparation of Compound 121

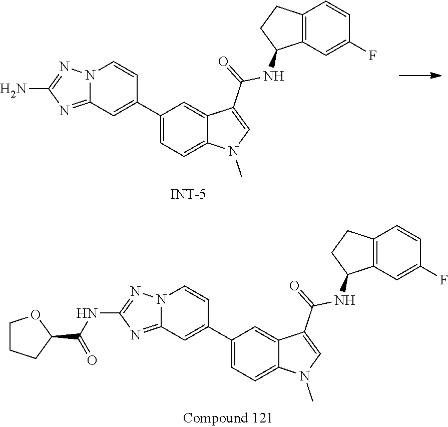 [SMILES: Cn1cc(C(=O)N[C@H]2CCc3ccc(F)cc32)c2cc(-c3ccn4nc(N)nc4c3)ccc21 ; "INT-5" ; Cn1cc(C(=O)N[C@H]2CCc3ccc(F)cc32)c2cc(-c3ccn4nc(NC(=O)[C@H]5CCCO5)nc4c3)ccc21]

### Preparation of Compound 121

INT-5 (88 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 121 with a yield of 71%. MS (ESI, positive ion) m/z: 539.2 [M+H]⁺.

¹H NMR (400 MHz, DMSO-d₆) δ 10.47 (s, 1H), 8.91 (dd, J=7.2, 0.8 Hz, 1H), 8.65 (d, J=1.9 Hz, 1H), 8.38 (d, J=8.2 Hz, 1H), 8.18 (s, 1H), 7.95 (dd, J=1.9, 0.9 Hz, 1H), 7.76 (dd, J=8.7, 1.9 Hz, 1H), 7.69 (d, J=8.6 Hz, 1H), 7.53 (dd, J=7.2, 2.0 Hz, 1H), 7.32 (dd, J=8.1, 5.4 Hz, 1H), 7.06 (td, J=8.6, 7.6, 1.7 Hz, 2H), 5.60 (q, J=8.1 Hz, 1H), 4.54 (s, 1H), 4.07-3.94 (m, 1H), 3.90 (s, 3H), 3.88-3.82 (m, 1H), 3.05-2.94 (m, 1H), 2.85 (dt, J=16.1, 8.5 Hz, 1H), 2.56 (dd, J=7.9, 3.1 Hz, 1H), 2.32-2.16 (m, 1H), 2.09-1.88 (m, 4H).

### Example 122: Preparation of Compound 122

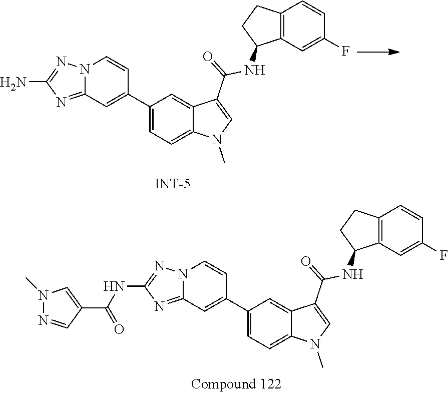 [SMILES: Cn1cc(C(=O)N[C@H]2CCc3ccc(F)cc32)c2cc(-c3ccn4nc(N)nc4c3)ccc21 ; "INT-5" ; Cn1cc(C(=O)Nc2nc3cc(-c4ccc5c(c4)c(C(=O)N[C@H]4CCc6ccc(F)cc64)cn5C)ccn3n2)cn1]

### Preparation of Compound 122

Intermediate INT-5 (88 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 122 with a yield of 44%. MS (ESI, positive ion) m/z: 549.2 [M+H]⁺.

¹H NMR (400 MHz, DMSO-d₆) δ 10.92 (s, 1H), 8.93 (d, J=7.1 Hz, 1H), 8.66 (d, J=1.8 Hz, 1H), 8.44 (s, 1H), 8.39 (d, J=8.3 Hz, 1H), 8.18 (s, 1H), 8.14 (s, 1H), 7.95 (d, J=1.9 Hz, 1H), 7.77 (dd, J=8.6, 1.9 Hz, 1H), 7.69 (d, J=8.6 Hz, 1H), 7.53 (dd, J=7.2, 2.0 Hz, 1H), 7.31 (dd, J=8.8, 5.2 Hz, 1H), 7.06 (t, J=7.8 Hz, 2H), 5.62 (t, J=8.1 Hz, 1H), 3.91 (d, J=7.9 Hz, 6H), 3.05-2.94 (m, 1H), 2.85 (dt, J=16.4, 8.7 Hz, 1H), 2.57 (d, J=8.8 Hz, 1H), 2.05 (dq, J=12.3, 8.8 Hz, 1H).

### Example 123: Preparation of Compound 123

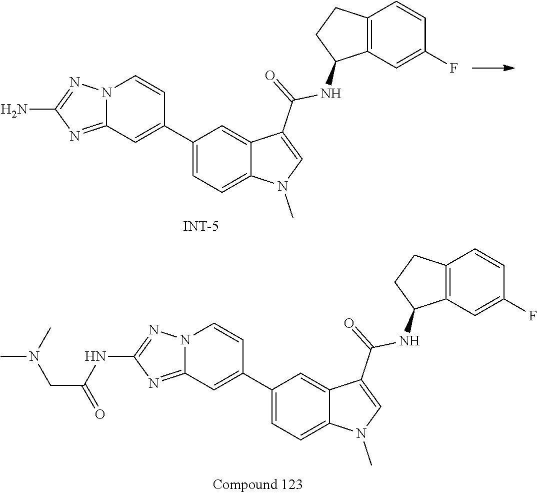 [SMILES: Cn1cc(C(=O)N[C@H]2CCc3ccc(F)cc32)c2cc(-c3ccn4nc(N)nc4c3)ccc21 ; "INT-5" ; CN(C)CC(=O)Nc1nc2cc(-c3ccc4c(c3)c(C(=O)N[C@H]3CCc5ccc(F)cc53)cn4C)ccn2n1]

### Preparation of Compound 123

Intermediate INT-5 (88 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 123 with a yield of 54%. MS (ESI, positive ion) m/z: 526.2 [M+H]⁺.

¹H NMR (400 MHz, DMSO-d₆) δ 10.45 (s, 1H), 8.91 (d, J=7.1 Hz, 1H), 8.65 (d, J=1.9 Hz, 1H), 8.38 (d, J=8.2 Hz, 1H), 8.18 (s, 1H), 7.97-7.92 (m, 1H), 7.77 (dd, J=8.6, 1.9 Hz, 1H), 7.69 (d, J=8.7 Hz, 1H), 7.52 (dd, J=7.1, 2.0 Hz, 1H), 7.36-7.28 (m, 1H), 7.06 (d, J=9.1 Hz, 2H), 5.61 (q, J=8.1 Hz, 1H), 3.90 (s, 3H), 3.22 (s, 2H), 3.05-2.95 (m, 1H), 2.86 (dt, J=16.0, 8.5 Hz, 1H), 2.56 (s, 1H), 2.33 (s, 6H), 2.05 (dq, J=12.5, 8.8 Hz, 1H).

### Example 124: Preparation of Compound 124

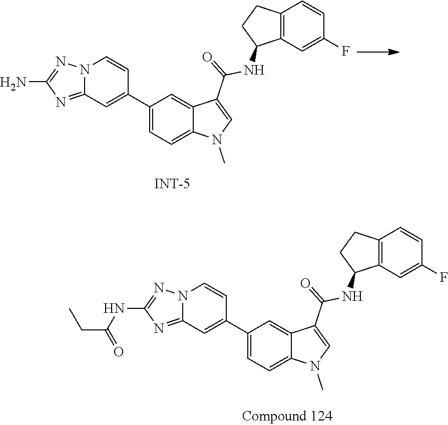 [SMILES: Cn1cc(C(=O)N[C@H]2CCc3ccc(F)cc32)c2cc(-c3ccn4nc(N)nc4c3)ccc21 ; "INT-5" ; CCC(=O)Nc1nc2cc(-c3ccc4c(c3)c(C(=O)N[C@H]3CCc5ccc(F)cc53)cn4C)ccn2n1]

### Preparation of Compound 124

Intermediate INT-5 (88 mg, 0.2 mmol) was dissolved in pyridine (1 mL); acetic acid (18 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 124 with a yield of 55%. MS (ESI, positive ion) m/z: 483.2 [M+H]⁺.

¹H NMR (400 MHz, DMSO-d₆) δ 10.79 (s, 1H), 8.89 (dd, J=7.1, 0.9 Hz, 1H), 8.64 (d, J=1.9 Hz, 1H), 8.37 (d, J=8.2 Hz, 1H), 8.17 (s, 1H), 7.93 (dd, J=2.1, 0.9 Hz, 1H), 7.76 (dd, J=8.6, 1.9 Hz, 1H), 7.69 (d, J=8.7 Hz, 1H), 7.50 (dd, J=7.1, 2.0 Hz, 1H), 7.35-7.29 (m, 1H), 7.06 (t, J=8.4 Hz, 2H), 5.60 (q, J=8.1 Hz, 1H), 3.90 (s, 3H), 3.00 (dd, J=13.8, 8.6 Hz, 1H), 2.86 (dt, J=16.0, 8.3 Hz, 1H), 2.55 (d, J=5.6 Hz, 1H), 2.18 (s, 3H), 2.05 (dq, J=12.4, 8.8 Hz, 1H).

### Example 125: Preparation of Compound 125

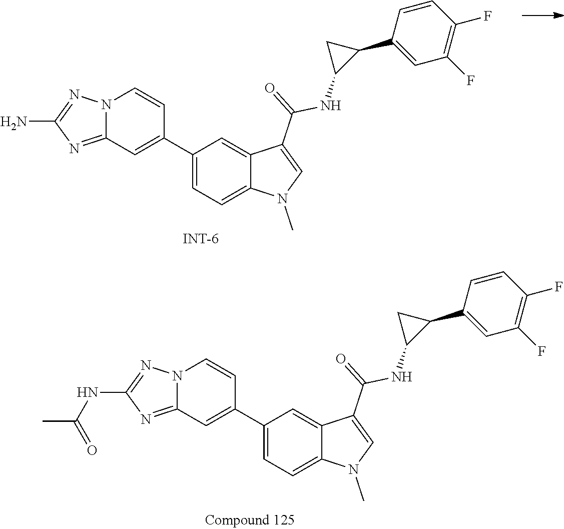 [SMILES: Cn1cc(C(=O)N[C@@H]2C[C@H]2c2ccc(F)c(F)c2)c2cc(-c3ccn4nc(N)nc4c3)ccc21 ; "INT-6" ; CC(=O)Nc1nc2cc(-c3ccc4c(c3)c(C(=O)N[C@@H]3C[C@H]3c3ccc(F)c(F)c3)cn4C)ccn2n1]

### Preparation of Compound 125

Intermediate INT-6 (91 mg, 0.2 mmol) was dissolved in pyridine (1 mL); acetic acid (18 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 125 with a yield of 55%. MS (ESI, positive ion) m/z: 501.2 [M+H]⁺.

¹H NMR (400 MHz, DMSO-d₆) δ 10.79 (s, 1H), 8.88 (dd, J=7.1, 0.8 Hz, 1H), 8.57 (d, J=1.8 Hz, 1H), 8.30 (d, J=3.8 Hz, 1H), 8.07 (s, 1H), 7.91 (dd, J=2.0, 0.9 Hz, 1H), 7.75 (dd, J=8.6, 1.9 Hz, 1H), 7.67 (d, J=8.7 Hz, 1H), 7.48 (dd, J=7.2, 2.0 Hz, 1H), 7.36 (dt, J=10.9, 8.6 Hz, 1H), 7.28 (ddd, J=12.2, 7.8, 2.2 Hz, 1H), 7.13-7.06 (m, 1H), 3.90 (s, 3H), 3.06-3.00 (m, 1H), 2.21 (d, J=21.2 Hz, 3H), 2.13-2.05 (m, 1H), 1.41-1.35 (m, 1H), 1.33-1.23 (m, 1H).

### Example 126: Preparation of Compound 126

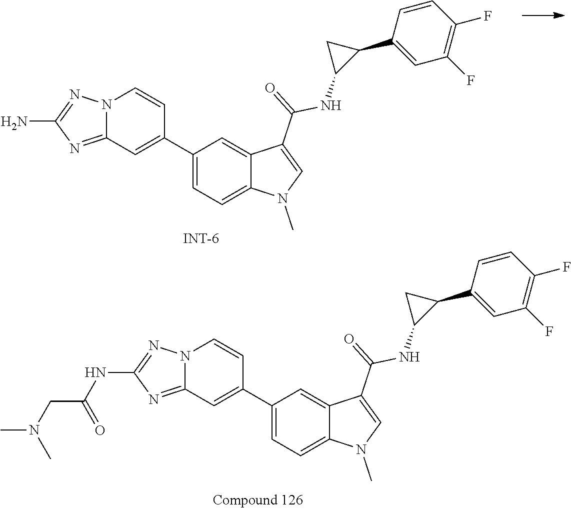 [SMILES: Cn1cc(C(=O)N[C@@H]2C[C@H]2c2ccc(F)c(F)c2)c2cc(-c3ccn4nc(N)nc4c3)ccc21 ; "INT-6" ; CN(C)CC(=O)Nc1nc2cc(-c3ccc4c(c3)c(C(=O)N[C@@H]3C[C@H]3c3ccc(F)c(F)c3)cn4C)ccn2n1]

### Preparation of Compound 126

Intermediate INT-6 (91 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 126 with a yield of 54%. MS (ESI, positive ion) m/z: 544.2 [M+H]⁺.

¹H NMR (400 MHz, DMSO-d₆) δ 10.45 (s, 1H), 8.93-8.86 (m, 1H), 8.57 (d, J=1.9 Hz, 1H), 8.30 (d, J=3.9 Hz, 1H), 8.07 (s, 1H), 7.92 (dd, J=2.0, 0.8 Hz, 1H), 7.75 (dd, J=8.7, 1.9 Hz, 1H), 7.68 (d, J=8.6 Hz, 1H), 7.50 (dd, J=7.2, 2.0 Hz, 1H), 7.42-7.23 (m, 2H), 7.10 (s, 1H), 3.91 (s, 3H), 3.21 (s, 2H), 3.02 (dd, J=8.0, 4.2 Hz, 1H), 2.33 (s, 6H), 2.13-2.04 (m, 1H), 1.38 (dt, J=9.8, 5.3 Hz, 1H), 1.33-1.24 (m, 1H).

### Example 127: Preparation of Compound 127

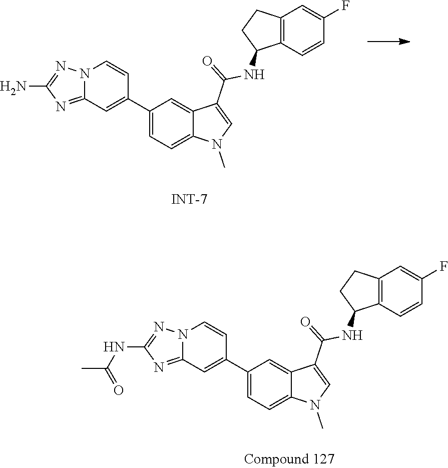 [SMILES: Cn1cc(C(=O)N[C@H]2CCc3cc(F)ccc32)c2cc(-c3ccn4nc(N)nc4c3)ccc21 ; "INT-7" ; CC(=O)Nc1nc2cc(-c3ccc4c(c3)c(C(=O)N[C@H]3CCc5cc(F)ccc53)cn4C)ccn2n1]

### Preparation of Compound 127

Intermediate INT-7 (88 mg, 0.2 mmol) was dissolved in pyridine (1 mL); acetic acid (18 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 127 with a yield of 53%. MS (ESI, positive ion) m/z: 483.2 [M+H]⁺.

¹H NMR (400 MHz, DMSO-d₆) δ 10.80 (s, 1H), 8.90 (d, J=7.0 Hz, 1H), 8.64 (d, J=1.9 Hz, 1H), 8.33 (d, J=8.3 Hz, 1H), 8.16 (s, 1H), 7.95-7.90 (m, 1H), 7.76 (dd, J=8.6, 1.9 Hz, 1H), 7.68 (d, J=8.7 Hz, 1H), 7.50 (dd, J=7.1, 2.0 Hz, 1H), 7.31 (dd, J=8.4, 5.4 Hz, 1H), 7.13 (d, J=9.5 Hz, 1H), 7.03 (t, J=8.8 Hz, 1H), 5.58 (q, J=8.0 Hz, 1H), 3.89 (s, 3H), 3.03 (dd, J=13.2, 10.0 Hz, 1H), 2.89 (dt, J=16.3, 8.5 Hz, 1H), 2.60-2.54 (m, 1H), 2.18 (s, 3H), 2.05 (dq, J=12.6, 8.7 Hz, 1H).

### Example 128: Preparation of Compound 128

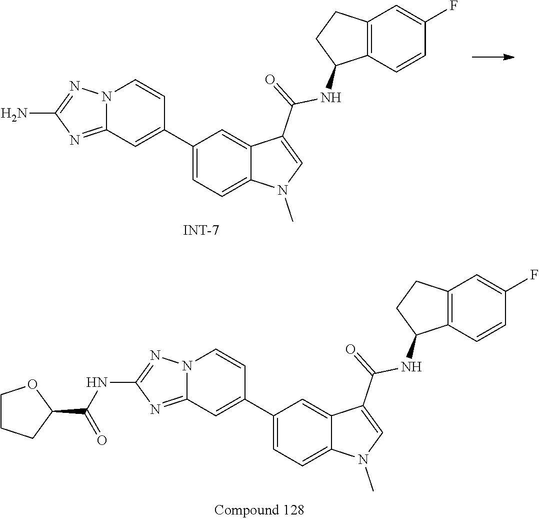 [SMILES: Cn1cc(C(=O)N[C@H]2CCc3cc(F)ccc32)c2cc(-c3ccn4nc(N)nc4c3)ccc21 ; "INT-7" ; Cn1cc(C(=O)N[C@H]2CCc3cc(F)ccc32)c2cc(-c3ccn4nc(NC(=O)[C@H]5CCCO5)nc4c3)ccc21]

### Preparation of Compound 128

INT-7 (88 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 128 with a yield of 71%. MS (ESI, positive ion) m/z: 539.2 [M+H]⁺.

¹H NMR (400 MHz, DMSO-d₆) δ 10.47 (s, 1H), 8.92 (d, J=7.1 Hz, 1H), 8.64 (d, J=1.8 Hz, 1H), 8.33 (d, J=8.3 Hz, 1H), 8.16 (s, 1H), 7.97-7.92 (m, 1H), 7.76 (dd, J=8.6, 1.9 Hz, 1H), 7.68 (d, J=8.7 Hz, 1H), 7.53 (dd, J=7.2, 2.0 Hz, 1H), 7.31 (dd, J=8.3, 5.4 Hz, 1H), 7.13 (d, J=9.4 Hz, 1H), 7.03 (t, J=8.9 Hz, 1H), 5.58 (q, J=7.9 Hz, 1H), 4.54 (s, 1H), 4.02 (q, J=7.0 Hz, 1H), 3.89 (s, 3H), 3.85 (q, J=7.2 Hz, 1H), 3.09-2.99 (m, 1H), 2.89 (dt, J=16.3, 8.5 Hz, 1H), 2.59-2.54 (m, 1H), 2.31-2.16 (m, 1H), 2.11-1.83 (m, 4H).

### Example 129: Preparation of Compound 129

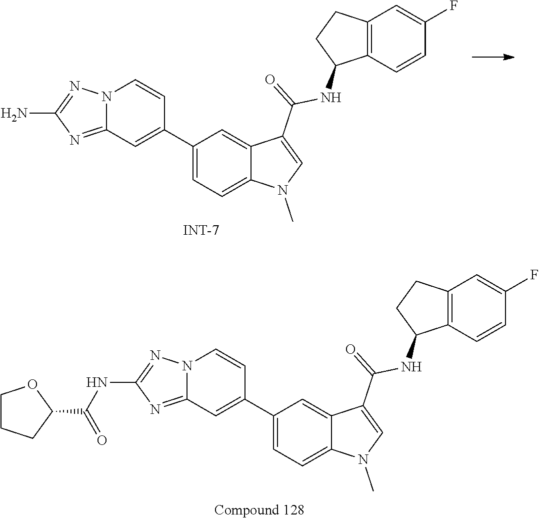 [SMILES: Cn1cc(C(=O)N[C@H]2CCc3cc(F)ccc32)c2cc(-c3ccn4nc(N)nc4c3)ccc21 ; "INT-7" ; Cn1cc(C(=O)N[C@H]2CCc3cc(F)ccc32)c2cc(-c3ccn4nc(NC(=O)[C@@H]5CCCO5)nc4c3)ccc21]

### Preparation of Compound 129

INT-7 (88 mg, 0.2 mmol) was dissolved in pyridine (1 mL), (S)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 129 with a yield of 61%. MS (ESI, positive ion) m/z: 539.2 [M+H]⁺.

¹H NMR (400 MHz, DMSO-d₆) δ 10.48 (s, 1H), 8.95-8.89 (m, 1H), 8.65 (d, J=1.9 Hz, 1H), 8.33 (d, J=8.3 Hz, 1H), 8.16 (s, 1H), 7.95 (dd, J=2.0, 0.9 Hz, 1H), 7.77 (dd, J=8.6, 1.9 Hz, 1H), 7.69 (d, J=8.6 Hz, 1H), 7.53 (dd, J=7.2, 2.0 Hz, 1H), 7.31 (dd, J=8.3, 5.4 Hz, 1H), 7.13 (d, J=9.0 Hz, 1H), 7.03 (t, J=8.9 Hz, 1H), 5.58 (q, J=8.0 Hz, 1H), 4.54 (s, 1H), 4.02 (q, J=7.0 Hz, 1H), 3.89 (s, 3H), 3.85 (q, J=7.0 Hz, 1H), 3.09-2.99 (m, 1H), 2.89 (dt, J=16.2, 8.4 Hz, 1H), 2.58-2.54 (m, 1H), 2.25 (dq, J=11.3, 7.8 Hz, 1H), 2.09-1.87 (m, 4H).

### Example 130: Preparation of Compound 130

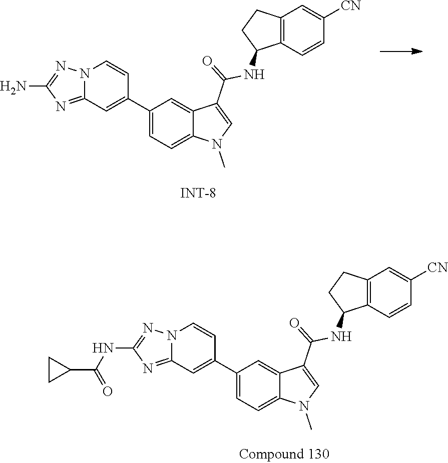 [SMILES: Cn1cc(C(=O)N[C@H]2CCc3cc(C#N)ccc32)c2cc(-c3ccn4nc(N)nc4c3)ccc21 ; "INT-8" ; Cn1cc(C(=O)N[C@H]2CCc3cc(C#N)ccc32)c2cc(-c3ccn4nc(NC(=O)C5CC5)nc4c3)ccc21]

### Preparation of Compound 130

INT-8 (89 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 130 with a yield of 61%. MS (ESI, positive ion) m/z: 516.2 [M+H]⁺.

¹H NMR (400 MHz, DMSO-d₆) δ 11.08 (s, 1H), 8.88 (d, J=7.0 Hz, 1H), 8.63 (d, J=1.9 Hz, 1H), 8.43 (d, J=8.2 Hz, 1H), 8.15 (s, 1H), 7.92 (d, J=1.9 Hz, 1H), 7.80-7.73 (m, 2H), 7.69 (dd, J=8.5, 3.1 Hz, 2H), 7.53-7.44 (m, 2H), 5.66 (q, J=8.5 Hz, 1H), 3.90 (s, 3H), 3.13-3.03 (m, 1H), 2.95 (dt, J=16.4, 8.7 Hz, 1H), 2.14-2.02 (m, 2H), 1.30 (ddd, J=4.0, 3.0, 2.0 Hz, 1H), 0.86 (d, J=6.1 Hz, 4H).

### Example 131: Preparation of Compound 131

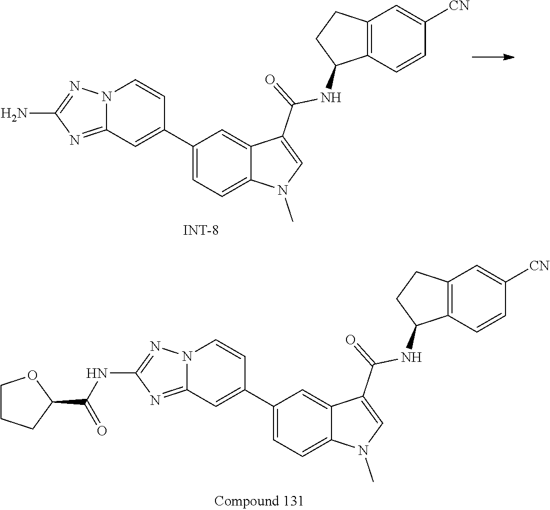 [SMILES: Cn1cc(C(=O)N[C@H]2CCc3cc(C#N)ccc32)c2cc(-c3ccn4nc(N)nc4c3)ccc21 ; "INT-8" ; Cn1cc(C(=O)N[C@H]2CCc3cc(C#N)ccc32)c2cc(-c3ccn4nc(NC(=O)[C@H]5CCCO5)nc4c3)ccc21]

### Preparation of Compound 131

INT-8 (89 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 131 with a yield of 71%. MS (ESI, positive ion) m/z: 546.2 [M+H]⁺.

¹H NMR (400 MHz, DMSO-d₆) δ 10.47 (s, 1H), 8.92 (d, J=7.3 Hz, 1H), 8.64 (d, J=1.9 Hz, 1H), 8.43 (d, J=8.3 Hz, 1H), 8.15 (s, 1H), 7.95 (dd, J=2.0, 0.8 Hz, 1H), 7.81-7.74 (m, 2H), 7.73-7.65 (m, 2H), 7.53 (dd, J=7.2, 2.0 Hz, 1H), 7.48 (d, J=7.9 Hz, 1H), 5.66 (q, J=8.3 Hz, 1H), 4.54 (s, 1H), 4.02 (q, J=7.0 Hz, 1H), 3.90 (s, 3H), 3.85 (q, J=7.0 Hz, 1H), 3.14-3.03 (m, 1H), 2.95 (dt, J=16.5, 8.7 Hz, 1H), 2.59-2.54 (m, 1H), 2.32-2.18 (m, 1H), 2.14-1.83 (m, 4H).

### Example 132: Preparation of Compound 132

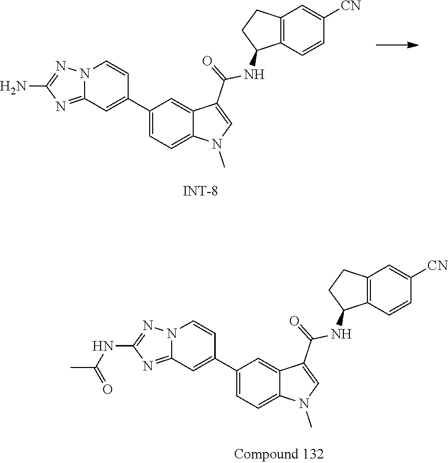 [SMILES: Cn1cc(C(=O)N[C@H]2CCc3cc(C#N)ccc32)c2cc(-c3ccn4nc(N)nc4c3)ccc21 ; "INT-8" ; CC(=O)Nc1nc2cc(-c3ccc4c(c3)c(C(=O)N[C@H]3CCc5cc(C#N)ccc53)cn4C)ccn2n1]

### Preparation of Compound 132

Intermediate INT-8 (89 mg, 0.2 mmol) was dissolved in pyridine (1 mL); acetic acid (18 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 132 with a yield of 53%. MS (ESI, positive ion) m/z: 490.2 [M+H]⁺.

¹H NMR (400 MHz, DMSO-d₆) δ 10.80 (s, 1H), 8.90 (dd, J=7.1, 0.8 Hz, 1H), 8.63 (d, J=2.1 Hz, 1H), 8.43 (d, J=8.3 Hz, 1H), 8.15 (s, 1H), 7.93 (dd, J=1.9, 0.9 Hz, 1H), 7.80-7.73 (m, 2H), 7.69 (dd, J=8.9, 2.8 Hz, 2H), 7.53-7.44 (m, 2H), 5.66 (q, J=8.3 Hz, 1H), 3.90 (s, 3H), 3.13-3.03 (m, 1H), 2.95 (dt, J=16.2, 8.5 Hz, 1H), 2.57 (dd, J=8.1, 2.7 Hz, 1H), 2.18 (s, 3H), 2.07 (dq, J=12.4, 8.9 Hz, 1H).

### Example 133: Preparation of Compound 133

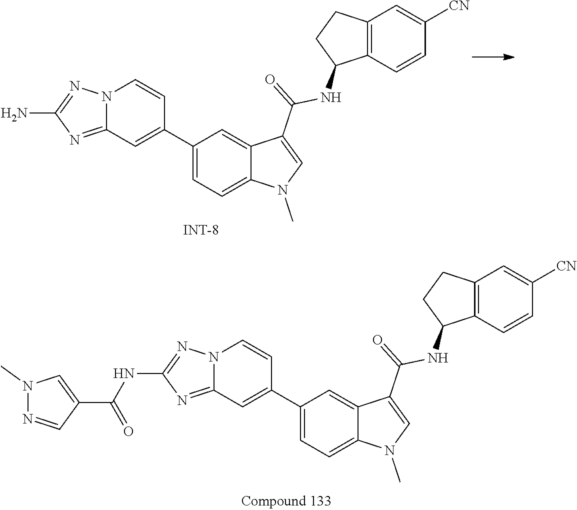 [SMILES: Cn1cc(C(=O)N[C@H]2CCc3cc(C#N)ccc32)c2cc(-c3ccn4nc(N)nc4c3)ccc21 ; "INT-8" ; Cn1cc(C(=O)Nc2nc3cc(-c4ccc5c(c4)c(C(=O)N[C@H]4CCc6cc(C#N)ccc64)cn5C)ccn3n2)cn1]

### Preparation of Compound 133

Intermediate INT-8 (89 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 133 with a yield of 44%. MS (ESI, positive ion) m/z: 556.2 [M+H]⁺.

¹H NMR (400 MHz, DMSO-d₆) δ 10.92 (s, 1H), 8.94 (dd, J=7.1, 0.8 Hz, 1H), 8.65 (d, J=2.0 Hz, 1H), 8.44 (d, J=7.3 Hz, 2H), 8.18-8.07 (m, 2H), 7.96 (dd, J=2.0, 0.9 Hz, 1H), 7.82-7.75 (m, 2H), 7.73-7.65 (m, 2H), 7.53 (dd, J=7.2, 2.0 Hz, 1H), 7.48 (d, J=7.8 Hz, 1H), 5.66 (q, J=8.4 Hz, 1H), 3.92 (s, 3H), 3.91 (s, 3H), 3.08 (dd, J=15.5, 9.1 Hz, 1H), 2.95 (dt, J=16.5, 8.7 Hz, 1H), 2.60-2.54 (m, 1H), 2.14-2.00 (m, 1H).

### Example 134: Preparation of Compound 134

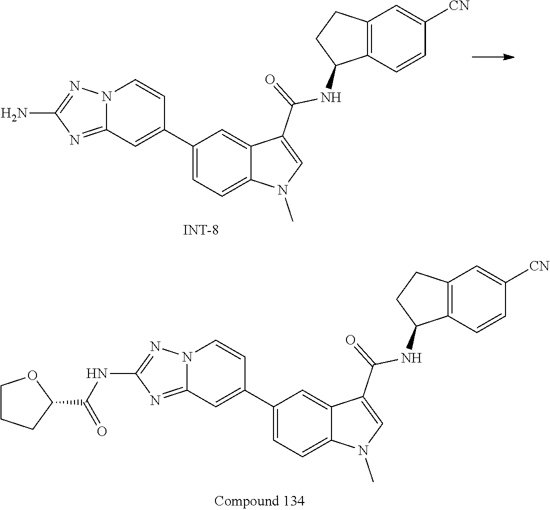 [SMILES: Cn1cc(C(=O)N[C@H]2CCc3cc(C#N)ccc32)c2cc(-c3ccn4nc(N)nc4c3)ccc21 ; "INT-8" ; Cn1cc(C(=O)N[C@H]2CCc3cc(C#N)ccc32)c2cc(-c3ccn4nc(NC(=O)[C@@H]5CCCO5)nc4c3)ccc21]

### Preparation of Compound 134

INT-8 (89 mg, 0.2 mmol) was dissolved in pyridine (1 mL), (S)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 134 with a yield of 61%. MS (ESI, positive ion) m/z: 546.2 [M+H]⁺.

¹H NMR (400 MHz, DMSO-d₆) δ 10.49 (s, 1H), 8.93 (d, J=7.1 Hz, 1H), 8.64 (d, J=1.8 Hz, 1H), 8.45 (d, J=8.2 Hz, 1H), 8.16 (s, 1H), 7.98-7.93 (m, 1H), 7.81-7.74 (m, 2H), 7.73-7.66 (m, 2H), 7.53 (dd, J=7.1, 2.0 Hz, 1H), 7.48 (d, J=7.8 Hz, 1H), 5.67 (q, J=8.3 Hz, 1H), 4.54 (s, 1H), 4.02 (q, J=6.9 Hz, 1H), 3.91 (s, 3H), 3.85 (q, J=7.1 Hz, 1H), 3.14-3.03 (m, 1H), 2.95 (dt, J=16.4, 8.6 Hz, 1H), 2.60-2.55 (m, 1H), 2.32-2.19 (m, 1H), 2.14-1.84 (m, 4H).

### Example 135: Preparation of Compound 135

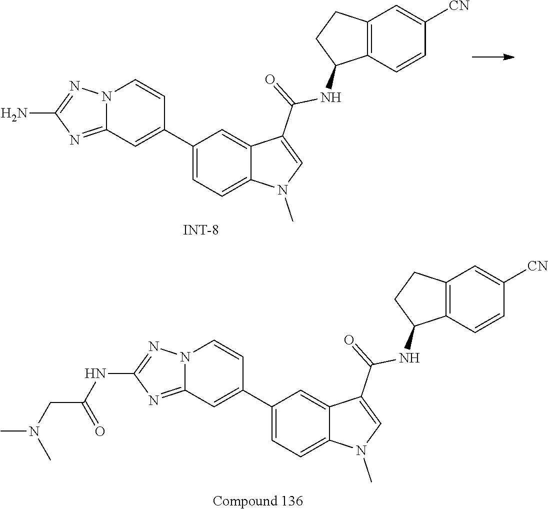 [SMILES: Cn1cc(C(=O)N[C@H]2CCc3cc(C#N)ccc32)c2cc(-c3ccn4nc(N)nc4c3)ccc21 ; "INT-8" ; CN(C)CC(=O)Nc1nc2cc(-c3ccc4c(c3)c(C(=O)N[C@H]3CCc5cc(C#N)ccc53)cn4C)ccn2n1]

### Preparation of Compound 135

Intermediate INT-8 (89 mg, 0.2 mmol) was dissolved in pyridine (1 mL); 2-dimethylaminoacetic acid (31 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 135 with a yield of 54%. MS (ESI, positive ion) m/z: 533.2 [M+H]⁺.

¹H NMR (400 MHz, DMSO-d₆) δ 8.93 (d, J=7.2 Hz, 1H), 8.64 (d, J=1.8 Hz, 1H), 8.45 (d, J=8.3 Hz, 1H), 8.16 (s, 1H), 7.99-7.94 (m, 1H), 7.81-7.74 (m, 2H), 7.73-7.66 (m, 2H), 7.55 (dd, J=7.2, 2.0 Hz, 1H), 7.47 (d, J=8.0 Hz, 1H), 5.66 (q, J=8.4 Hz, 1H), 3.90 (s, 3H), 3.72 (s, 2H), 3.08 (dd, J=15.1, 7.9 Hz, 1H), 2.95 (dt, J=16.5, 8.6 Hz, 1H), 2.60 (d, J=17.7 Hz, 6H), 2.58-2.55 (m, 1H), 2.14-2.00 (m, 1H).

### Example 136: Preparation of Compound 136

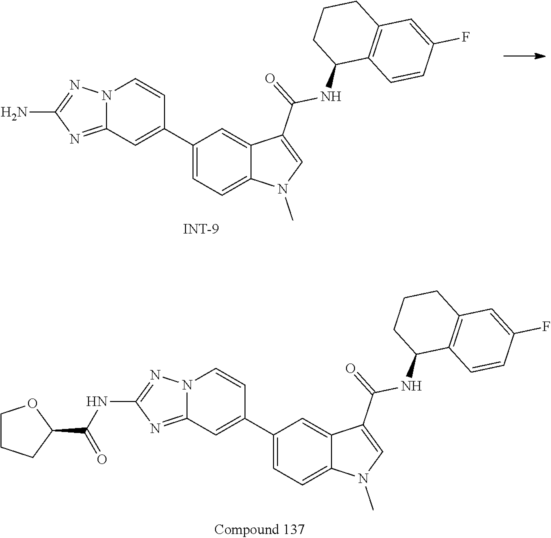 [SMILES: Cn1cc(C(=O)N[C@H]2CCCc3cc(F)ccc32)c2cc(-c3ccn4nc(N)nc4c3)ccc21 ; "INT-9" ; Cn1cc(C(=O)N[C@H]2CCCc3cc(F)ccc32)c2cc(-c3ccn4nc(NC(=O)[C@H]5CCCO5)nc4c3)ccc21]

### Preparation of Compound 136

INT-9 (90 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 136 with a yield of 61%. MS (ESI, positive ion) m/z: 523.2 [M+H]⁺.

¹H NMR (400 MHz, DMSO-d₆) δ 11.16 (s, 1H), 8.89 (d, J=7.1 Hz, 1H), 8.67 (d, J=1.8 Hz, 1H), 8.33 (d, J=8.6 Hz, 1H), 8.19 (s, 1H), 7.94 (d, J=2.4 Hz, 1H), 7.75 (dd, J=8.7, 1.9 Hz, 1H), 7.67 (d, J=8.6 Hz, 1H), 7.50 (dd, J=7.2, 2.0 Hz, 1H), 7.31 (dd, J=8.2, 6.1 Hz, 1H), 6.99 (t, J=8.8 Hz, 2H), 5.27 (q, J=8.4, 7.5 Hz, 1H), 3.88 (s, 3H), 2.91-2.73 (m, 2H), 2.00 (p, J=7.4, 6.5 Hz, 2H), 1.92-1.73 (m, 2H), 0.93-0.80 (m, 5H).

### Example 137: Preparation of Compound 137

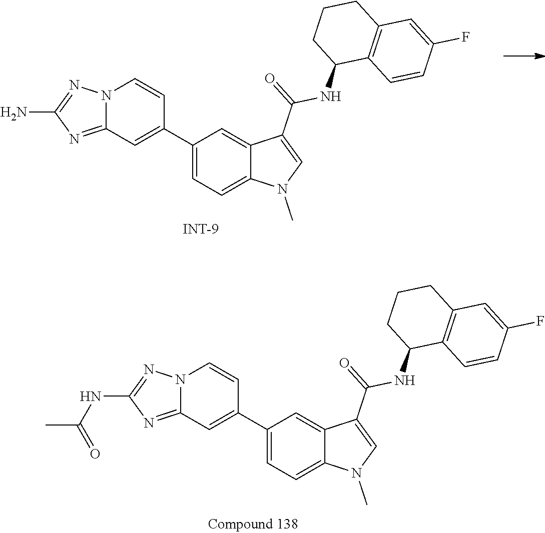 [SMILES: Cn1cc(C(=O)N[C@H]2CCCc3cc(F)ccc32)c2cc(-c3ccn4nc(N)nc4c3)ccc21 ; "INT-9" ; CC(=O)Nc1nc2cc(-c3ccc4c(c3)c(C(=O)N[C@H]3CCCc5cc(F)ccc53)cn4C)ccn2n1]

### Preparation of Compound 137

INT-9 (90 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 137 with a yield of 71%. MS (ESI, positive ion) m/z: 553.2 [M+H]⁺.

¹H NMR (400 MHz, DMSO-d₆) δ 10.48 (s, 1H), 8.92 (dd, J=7.2, 0.8 Hz, 1H), 8.66 (d, J=1.8 Hz, 1H), 8.32 (d, J=8.6 Hz, 1H), 8.18 (s, 1H), 7.94 (dd, J=2.0, 0.9 Hz, 1H), 7.76 (dd, J=8.6, 1.9 Hz, 1H), 7.68 (d, J=8.6 Hz, 1H), 7.52 (dd, J=7.1, 2.0 Hz, 1H), 7.36-7.27 (m, 1H), 6.99 (d, J=9.2 Hz, 2H), 5.27 (t, J=6.5 Hz, 1H), 4.02 (q, J=7.0 Hz, 1H), 3.89 (s, 3H), 3.88-3.80 (m, 2H), 2.83 (dt, J=7.3, 4.2 Hz, 2H), 1.99 (dt, J=15.2, 8.1 Hz, 4H), 1.91 (q, J=7.0 Hz, 2H), 1.84 (dd, J=18.7, 10.8 Hz, 2H).

### Example 138: Preparation of Compound 138

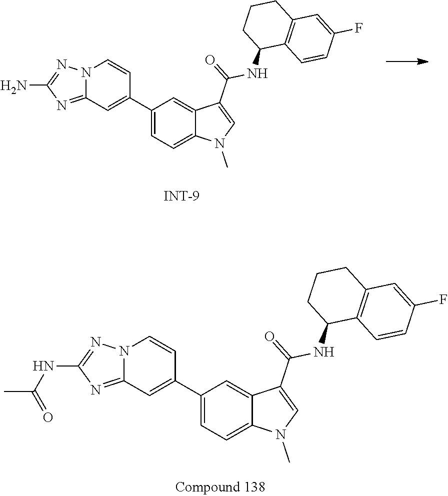 [SMILES: Cn1cc(C(=O)N[C@H]2CCCc3cc(F)ccc32)c2cc(-c3ccn4nc(N)nc4c3)ccc21 ; "INT-9" ; CC(=O)Nc1nc2cc(-c3ccc4c(c3)c(C(=O)N[C@H]3CCCc5cc(F)ccc53)cn4C)ccn2n1]

### Preparation of Compound 138

INT-9 (90 mg, 0.2 mmol) was dissolved in pyridine (1 mL), acetic acid (18 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 138 with a yield of 61%. MS (ESI, positive ion) m/z: 497.2 [M+H]⁺.

¹H NMR (400 MHz, DMSO-d₆) δ 10.80 (s, 1H), 8.90 (d, J=7.0 Hz, 1H), 8.65 (d, J=1.9 Hz, 1H), 8.32 (d, J=8.6 Hz, 1H), 8.17 (s, 1H), 7.95-7.90 (m, 1H), 7.76 (dd, J=8.7, 1.8 Hz, 1H), 7.68 (d, J=8.6 Hz, 1H), 7.50 (dd, J=7.1, 2.0 Hz, 1H), 7.36-7.28 (m, 1H), 7.00 (t, J=8.5 Hz, 2H), 5.27 (s, 1H), 3.89 (s, 3H), 2.82 (d, J=6.9 Hz, 2H), 2.18 (s, 3H), 2.00 (d, J=4.9 Hz, 2H), 1.91-1.75 (m, 2H).

### Example 139: Preparation of Compound 139

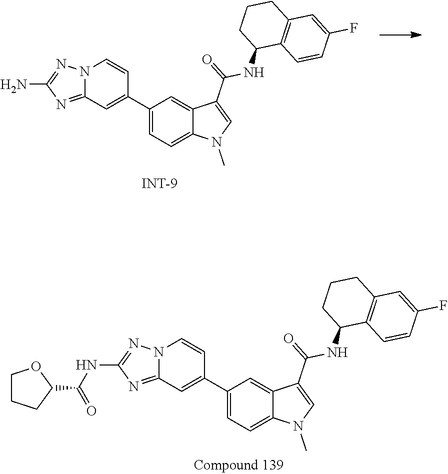

### Preparation of Compound 139

INT-9 (90 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 139 with a yield of 68%. MS (ESI, positive ion) m/z: 553.2 [M+H]⁺.

¹H NMR (400 MHz, DMSO-d₆) δ 10.48 (s, 1H), 8.92 (dd, J=7.2, 0.8 Hz, 1H), 8.66 (d, J=1.8 Hz, 1H), 8.32 (d, J=8.6 Hz, 1H), 8.18 (s, 1H), 7.94 (dd, J=2.0, 0.9 Hz, 1H), 7.76 (dd, J=8.6, 1.9 Hz, 1H), 7.68 (d, J=8.6 Hz, 1H), 7.52 (dd, J=7.1, 2.0 Hz, 1H), 7.36-7.27 (m, 1H), 6.99 (d, J=9.2 Hz, 2H), 5.27 (t, J=6.5 Hz, 1H), 4.02 (q, J=7.0 Hz, 1H), 3.89 (s, 3H), 3.88-3.80 (m, 2H), 2.83 (dt, J=7.3, 4.2 Hz, 2H), 1.99 (dt, J=15.2, 8.1 Hz, 4H), 1.91 (q, J=7.0 Hz, 2H), 1.84 (dd, J=18.7, 10.8 Hz, 2H).

### Example 140: Preparation of Compound 140

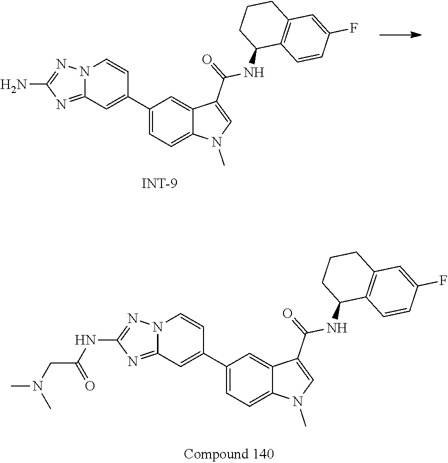 [SMILES: Cn1cc(C(=O)N[C@H]2CCCc3cc(F)ccc32)c2cc(-c3ccn4nc(N)nc4c3)ccc21 ; "INT-9" ; CN(C)CC(=O)Nc1nc2cc(-c3ccc4c(c3)c(C(=O)N[C@H]3CCCc5cc(F)ccc53)cn4C)ccn2n1]

### Preparation of Compound 140

INT-9 (90 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 140 with a yield of 56%. MS (ESI, positive ion) m/z: 540.2 [M+H]⁺.

¹H NMR (400 MHz, DMSO-d₆) δ 10.45 (s, 1H), 8.94-8.88 (m, 1H), 8.65 (d, J=1.8 Hz, 1H), 8.31 (d, J=8.6 Hz, 1H), 8.17 (s, 1H), 7.93 (dd, J=2.0, 0.9 Hz, 1H), 7.76 (dd, J=8.7, 1.9 Hz, 1H), 7.68 (d, J=8.7 Hz, 1H), 7.51 (dd, J=7.2, 2.0 Hz, 1H), 7.36-7.27 (m, 1H), 7.00 (t, J=8.5 Hz, 2H), 5.27 (s, 1H), 3.89 (s, 3H), 3.21 (s, 2H), 2.82 (d, J=6.4 Hz, 2H), 2.33 (s, 6H), 2.06-1.94 (m, 2H), 1.84 (td, J=16.8, 14.5, 9.2 Hz, 2H).

### Example 141: Preparation of Compound 141

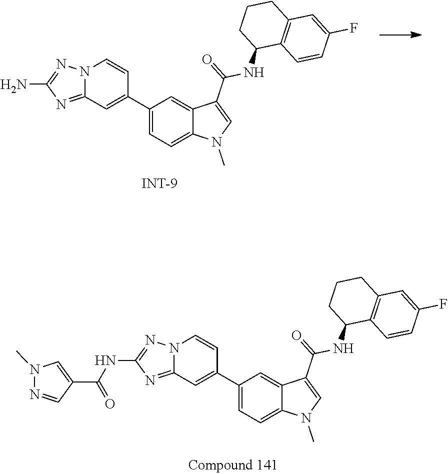 [SMILES: Cn1cc(C(=O)N[C@H]2CCCc3cc(F)ccc32)c2cc(-c3ccn4nc(N)nc4c3)ccc21 ; "INT-9" ; Cn1cc(C(=O)Nc2nc3cc(-c4ccc5c(c4)c(C(=O)N[C@H]4CCCc6cc(F)ccc64)cn5C)ccn3n2)cn1]

### Preparation of Compound 141

INT-9 (90 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 141 with a yield of 62%. MS (ESI, positive ion) m/z: 563.2 [M+H]⁺.

¹H NMR (400 MHz, DMSO-d₆) δ 10.93 (s, 1H), 8.94 (d, J=7.1 Hz, 1H), 8.67 (s, 1H), 8.44 (s, 1H), 8.33 (d, J=8.7 Hz, 1H), 8.18 (s, 1H), 8.13 (s, 1H), 7.98-7.93 (m, 1H), 7.81-7.74 (m, 1H), 7.69 (d, J=8.7 Hz, 1H), 7.52 (dd, J=7.3, 2.0 Hz, 1H), 7.36-7.28 (m, 1H), 6.99 (d, J=9.2 Hz, 2H), 5.28 (s, 1H), 3.92 (s, 3H), 3.89 (s, 3H), 2.82 (s, 2H), 2.66-2.56 (m, 1H), 2.00 (d, J=5.3 Hz, 2H), 1.83 (s, 1H).

### Example 142: Preparation of Compound 142

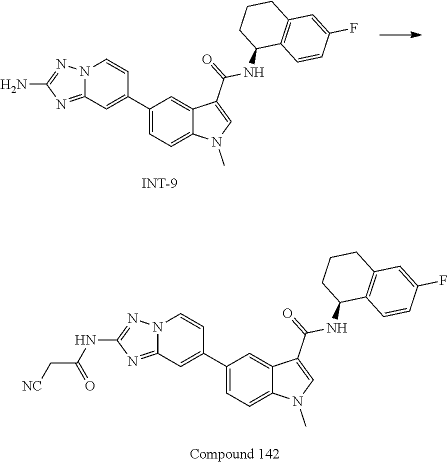 [SMILES: Cn1cc(C(=O)N[C@H]2CCCc3cc(F)ccc32)c2cc(-c3ccn4nc(N)nc4c3)ccc21 ; "INT-9" ; Cn1cc(C(=O)N[C@H]2CCCc3cc(F)ccc32)c2cc(-c3ccn4nc(NC(=O)CC#N)nc4c3)ccc21]

### Preparation of Compound 142

INT-9 (90 mg, 0.2 mmol) was dissolved in pyridine (1 mL), cyanoacetic acid (25 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 142 with a yield of 71%. MS (ESI, positive ion) m/z: 522.2 [M+H]⁺.

¹H NMR (400 MHz, DMSO-d₆) δ 11.27 (s, −1H), 8.94 (d, J=7.1 Hz, 1H), 8.66 (d, J=1.9 Hz, 1H), 8.32 (d, J=8.6 Hz, 1H), 8.17 (s, 1H), 7.97 (dd, J=2.1, 0.9 Hz, 1H), 7.77 (dd, J=8.7, 1.9 Hz, 1H), 7.68 (d, J=8.7 Hz, 1H), 7.54 (dd, J=7.1, 2.0 Hz, 1H), 7.36-7.28 (m, 1H), 7.00 (t, J=8.4 Hz, 2H), 5.27 (s, 1H), 3.90-3.88 (m, 3H), 2.82 (d, J=6.4 Hz, 2H), 2.80-2.57 (m, 1H), 2.50-2.26 (m, 1H), 2.04-1.97 (m, 2H), 1.84 (dq, J=15.1, 8.2, 5.6 Hz, 2H).

### Example 143: Preparation of Compound 143

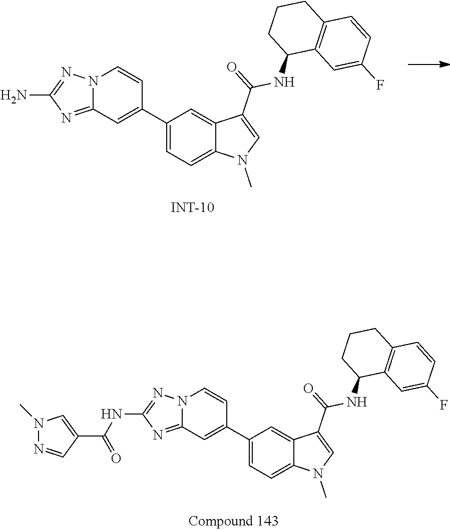 [SMILES: Cn1cc(C(=O)N[C@H]2CCCc3ccc(F)cc32)c2cc(-c3ccn4nc(N)nc4c3)ccc21 ; "INT-10" ; Cn1cc(C(=O)Nc2nc3cc(-c4ccc5c(c4)c(C(=O)N[C@H]4CCCc6ccc(F)cc64)cn5C)ccn3n2)cn1]

### Preparation of Compound 143

INT-10 (90 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 143 with a yield of 72%. MS (ESI, positive ion) m/z: 563.2 [M+H]⁺.

¹H NMR (400 MHz, DMSO-d₆) δ 10.92 (s, 1H), 8.94 (dd, J=7.1, 0.8 Hz, 1H), 8.67 (d, J=1.9 Hz, 1H), 8.44 (s, 1H), 8.32 (d, J=8.7 Hz, 1H), 8.18 (s, 1H), 8.13 (d, J=0.8 Hz, 1H), 7.95 (dd, J=2.0, 0.9 Hz, 1H), 7.77 (dd, J=8.7, 1.9 Hz, 1H), 7.69 (d, J=8.7 Hz, 1H), 7.52 (dd, J=7.2, 2.0 Hz, 1H), 7.32 (dd, J=8.2, 6.1 Hz, 1H), 7.00 (t, J=8.7 Hz, 2H), 5.31-5.23 (m, 1H), 3.92 (s, 3H), 3.89 (s, 3H), 2.82 (d, J=6.7 Hz, 2H), 2.01 (dd, J=9.0, 4.7 Hz, 2H), 1.83 (ddd, J=23.5, 11.1, 7.9 Hz, 2H).

### Example 144: Preparation of Compound 144

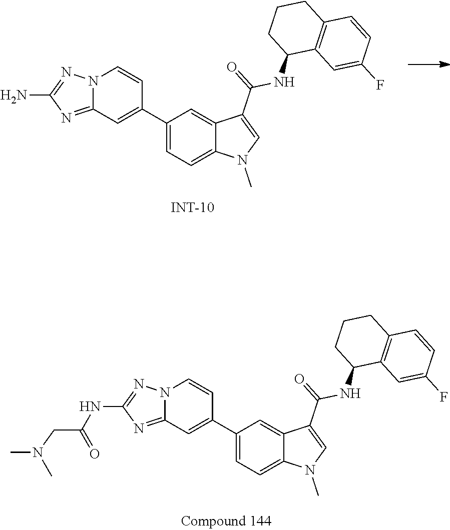 [SMILES: Cn1cc(C(=O)N[C@H]2CCCc3ccc(F)cc32)c2cc(-c3ccn4nc(N)nc4c3)ccc21 ; "INT-10" ; CN(C)CC(=O)Nc1nc2cc(-c3ccc4c(c3)c(C(=O)N[C@H]3CCCc5ccc(F)cc53)cn4C)ccn2n1]

### Preparation of Compound 144

INT-10 (90 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 144 with a yield of 58%. MS (ESI, positive ion) m/z: 540.2 [M+H]⁺.

¹H NMR (400 MHz, DMSO-d₆) δ 10.45 (s, 1H), 8.91 (d, J=7.0 Hz, 1H), 8.65 (d, J=1.8 Hz, 1H), 8.32 (d, J=8.6 Hz, 1H), 8.17 (s, 1H), 7.93 (dd, J=2.1, 0.9 Hz, 1H), 7.76 (dd, J=8.7, 1.9 Hz, 1H), 7.68 (d, J=8.6 Hz, 1H), 7.51 (dd, J=7.2, 2.0 Hz, 1H), 7.36-7.28 (m, 1H), 7.00 (t, J=8.4 Hz, 2H), 5.27 (s, 1H), 3.89 (s, 3H), 3.21 (s, 2H), 2.82 (d, J=6.4 Hz, 2H), 2.33 (s, 6H), 2.04-1.97 (m, 2H), 1.86 (dd, J=22.3, 13.8 Hz, 2H).

### Example 145: Preparation of Compound 145

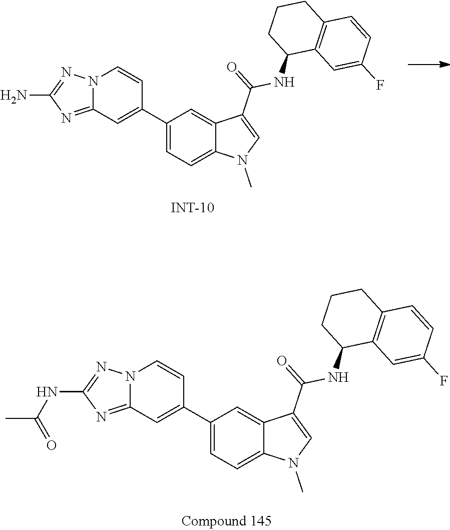 [SMILES: Cn1cc(C(=O)N[C@H]2CCCc3ccc(F)cc32)c2cc(-c3ccn4nc(N)nc4c3)ccc21 ; "INT-10" ; CC(=O)Nc1nc2cc(-c3ccc4c(c3)c(C(=O)N[C@H]3CCCc5ccc(F)cc53)cn4C)ccn2n1]

### Preparation of Compound 145

INT-10 (90 mg, 0.2 mmol) was dissolved in pyridine (1 mL); acetic acid (18 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 145 with a yield of 61%. MS (ESI, positive ion) m/z: 497.2 [M+H]⁺.

¹H NMR (400 MHz, DMSO-d₆) δ 10.82 (s, 1H), 8.90 (dd, J=7.1, 0.8 Hz, 1H), 8.66 (d, J=1.9 Hz, 1H), 8.33 (d, J=8.6 Hz, 1H), 8.18 (s, 1H), 7.93 (dd, J=1.9, 0.9 Hz, 1H), 7.76 (dd, J=8.6, 1.9 Hz, 1H), 7.68 (d, J=8.6 Hz, 1H), 7.50 (dd, J=7.1, 2.0 Hz, 1H), 7.36-7.27 (m, 1H), 7.00 (t, J=8.5 Hz, 2H), 5.27 (s, 1H), 3.89 (s, 3H), 2.82 (d, J=6.8 Hz, 2H), 2.18 (s, 3H), 2.06-1.95 (m, 2H), 1.85 (dt, J=17.8, 10.0 Hz, 2H).

### Example 146: Preparation of Compound 146

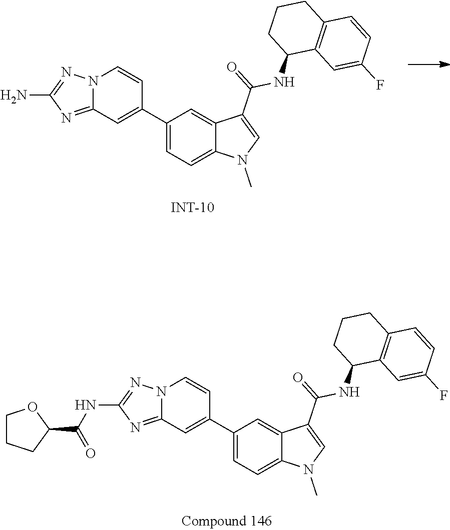 [SMILES: Cn1cc(C(=O)N[C@H]2CCCc3ccc(F)cc32)c2cc(-c3ccn4nc(N)nc4c3)ccc21 ; "INT-10" ; Cn1cc(C(=O)N[C@H]2CCCc3ccc(F)cc32)c2cc(-c3ccn4nc(NC(=O)[C@H]5CCCO5)nc4c3)ccc21]

### Preparation of Compound 146

INT-10 (90 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 146 with a yield of 68%. MS (ESI, positive ion) m/z: 553.2 [M+H]⁺.

¹H NMR (400 MHz, DMSO-d₆) δ 10.48 (s, 1H), 8.92 (d, J=7.1 Hz, 1H), 8.66 (d, J=1.8 Hz, 1H), 8.32 (d, J=8.6 Hz, 1H), 8.18 (s, 1H), 7.95 (d, J=2.0 Hz, 1H), 7.76 (dd, J=8.7, 1.9 Hz, 1H), 7.69 (d, J=8.6 Hz, 1H), 7.52 (dd, J=7.2, 2.0 Hz, 1H), 7.36-7.29 (m, 1H), 7.00 (t, J=8.4 Hz, 2H), 5.28 (s, 1H), 4.54 (s, 1H), 4.02 (q, J=7.0 Hz, 1H), 3.89 (s, 3H), 3.88-3.82 (m, 1H), 2.82 (d, J=6.6 Hz, 2H), 2.30-2.21 (m, 1H), 2.00 (q, J=6.5, 6.1 Hz, 3H), 1.88 (ddd, J=32.1, 17.2, 9.5 Hz, 4H).

### Example 147: Preparation of Compound 147

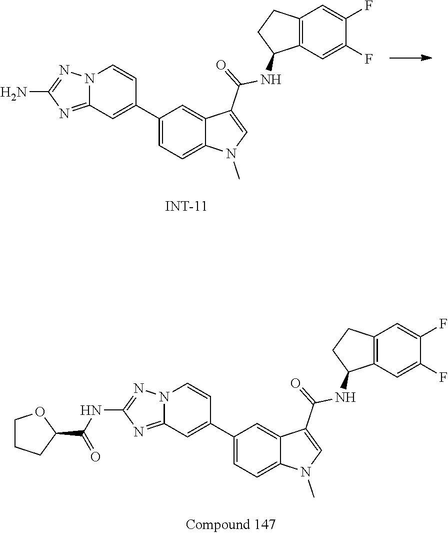 [SMILES: Cn1cc(C(=O)N[C@H]2CCc3cc(F)c(F)cc32)c2cc(-c3ccn4nc(N)nc4c3)ccc21 ; "INT-11" ; Cn1cc(C(=O)N[C@H]2CCc3cc(F)c(F)cc32)c2cc(-c3ccn4nc(NC(=O)[C@H]5CCCO5)nc4c3)ccc21]

### Preparation of Compound 147

INT-11 (90 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 147 with a yield of 68%. MS (ESI, positive ion) m/z: 553.2 [M+H]⁺.

¹H NMR (400 MHz, DMSO-d₆) δ 11.27 (s, −1H), 8.94 (d, J=7.1 Hz, 1H), 8.66 (d, J=1.9 Hz, 1H), 8.32 (d, J=8.6 Hz, 1H), 8.17 (s, 1H), 7.97 (dd, J=2.1, 0.9 Hz, 1H), 7.77 (dd, J=8.7, 1.9 Hz, 1H), 7.68 (d, J=8.7 Hz, 1H), 7.54 (dd, J=7.1, 2.0 Hz, 1H), 7.36-7.28 (m, 1H), 7.00 (t, J=8.4 Hz, 2H), 5.27 (s, 1H), 3.90-3.88 (m, 3H), 2.82 (d, J=6.4 Hz, 2H), 2.80-2.57 (m, 1H), 2.50-2.26 (m, 1H), 2.04-1.97 (m, 2H), 1.84 (dq, J=15.1, 8.2, 5.6 Hz, 2H).

### Example 148: Preparation of Compound 148

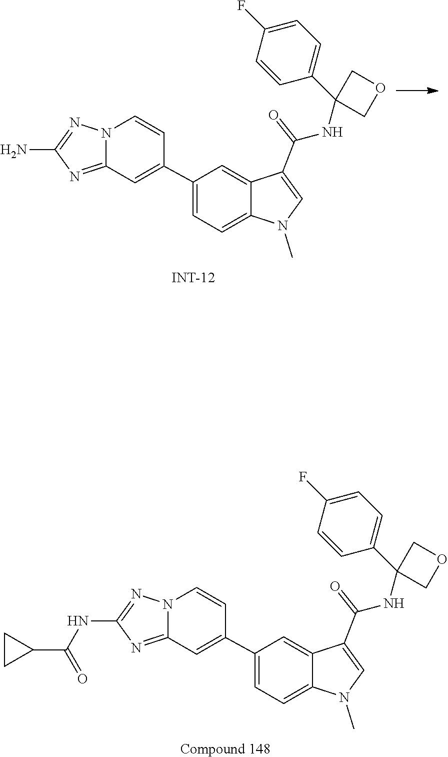 [SMILES: Cn1cc(C(=O)NC2(c3ccc(F)cc3)COC2)c2cc(-c3ccn4nc(N)nc4c3)ccc21 ; "INT-12" ; Cn1cc(C(=O)NC2(c3ccc(F)cc3)COC2)c2cc(-c3ccn4nc(NC(=O)C5CC5)nc4c3)ccc21 ; "Compound 148"]

### Preparation of Compound 148

INT-12 (91 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 148 with a yield of 61%. MS (ESI, positive ion) m/z: 525.2 [M+H]⁺.

¹H NMR (400 MHz, DMSO-d₆) δ 11.10 (s, 1H), 9.22 (s, 1H), 8.93 (s, 1H), 8.63 (d, J=1.8 Hz, 1H), 8.05 (s, 1H), 7.95 (d, J=1.9 Hz, 1H), 7.81 (dd, J=8.7, 1.9 Hz, 1H), 7.73 (d, J=8.6 Hz, 1H), 7.61-7.57 (m, 2H), 7.51 (dd, J=7.1, 2.0 Hz, 1H), 7.21 (d, J=8.9 Hz, 2H), 5.13 (t, J=5.9 Hz, 1H), 4.77 (d, J=8.1 Hz, 1H), 4.29 (d, J=8.1 Hz, 1H), 3.93 (s, 3H), 3.66 (d, J=6.5 Hz, 2H), 0.86 (d, J=6.2 Hz, 5H).

### Example 149: Preparation of Compound 149

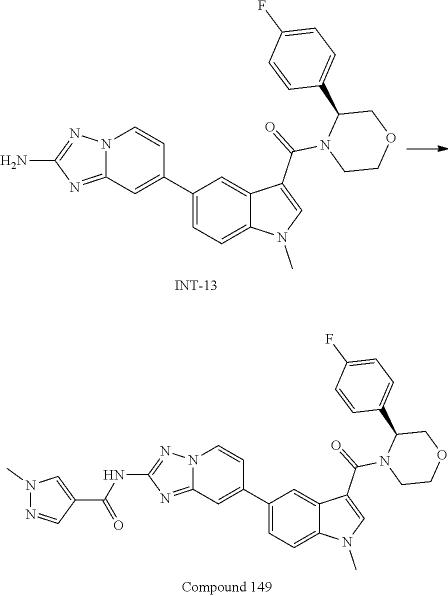 [SMILES: Cn1cc(C(=O)N2CCOC[C@@H]2c2ccc(F)cc2)c2cc(-c3ccn4nc(N)nc4c3)ccc21 ; "INT-13" ; Cn1cc(C(=O)Nc2nc3cc(-c4ccc5c(c4)c(C(=O)N4CCOC[C@@H]4c4ccc(F)cc4)cn5C)ccn3n2)cn1]

### Preparation of Compound 149

INT-13 (94 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 149 with a yield of 62%. MS (ESI, positive ion) m/z: 579.2 [M+H]⁺.

¹H NMR (400 MHz, DMSO-d₆) δ 10.92 (s, 1H), 8.95-8.85 (m, 1H), 8.44 (s, 1H), 8.19 (dd, J=9.3, 1.8 Hz, 1H), 8.13 (s, 1H), 7.97-7.92 (m, 1H), 7.88 (d, J=9.6 Hz, 1H), 7.78 (td, J=7.7, 6.8, 1.7 Hz, 1H), 7.73-7.64 (m, 1H), 7.61-7.38 (m, 4H), 7.24 (t, J=8.9 Hz, 2H), 5.63 (s, 1H), 4.50 (d, J=12.2 Hz, 1H), 4.10 (d, J=13.4 Hz, 1H), 3.98-3.93 (m, 1H), 3.92 (s, 3H), 3.90 (s, 3H), 3.87 (d, J=11.4 Hz, 2H), 3.68-3.58 (m, 1H).

### Example 150: Preparation of Compound 150

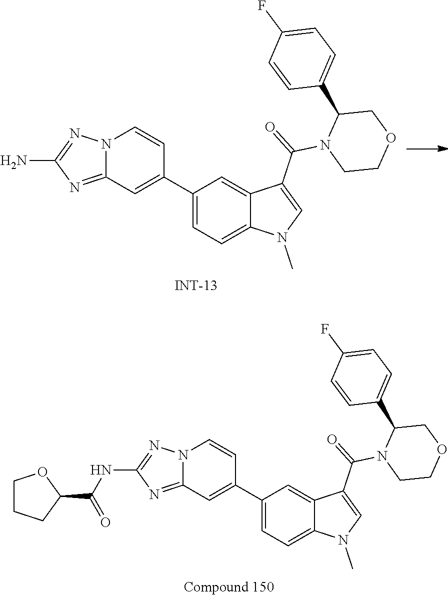 [SMILES: Cn1cc(C(=O)N2CCOC[C@@H]2c2ccc(F)cc2)c2cc(-c3ccn4nc(N)nc4c3)ccc21 ; "INT-13" ; Cn1cc(C(=O)N2CCOC[C@@H]2c2ccc(F)cc2)c2cc(-c3ccn4nc(NC(=O)[C@H]5CCCO5)nc4c3)ccc21]

### Preparation of Compound 150

INT-13 (94 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 150 with a yield of 66%. MS (ESI, positive ion) m/z: 579.2 [M+H]⁺.

¹H NMR (400 MHz, DMSO-d₆) δ 10.47 (s, 1H), 8.90 (d, J=7.2 Hz, 1H), 8.21-8.10 (m, 1H), 7.96-7.87 (m, 2H), 7.77 (dd, J=8.7, 1.8 Hz, 1H), 7.69 (d, J=8.6 Hz, 1H), 7.53 (dt, J=9.5, 4.7 Hz, 2H), 7.48 (dd, J=7.2, 2.0 Hz, 1H), 7.24 (t, J=8.9 Hz, 2H), 5.63 (s, 1H), 4.49 (d, J=12.3 Hz, 1H), 4.09 (d, J=13.5 Hz, 1H), 4.02 (q, J=7.0 Hz, 1H), 3.94 (dd, J=12.2, 3.5 Hz, 2H), 3.89 (s, 3H), 3.85 (q, J=7.1 Hz, 2H), 3.63 (td, J=11.7, 2.9 Hz, 1H), 3.29 (d, J=12.6 Hz, 1H), 2.32-1.95 (m, 2H), 1.94-1.80 (m, 2H).

### Example 151: Preparation of Compound 151

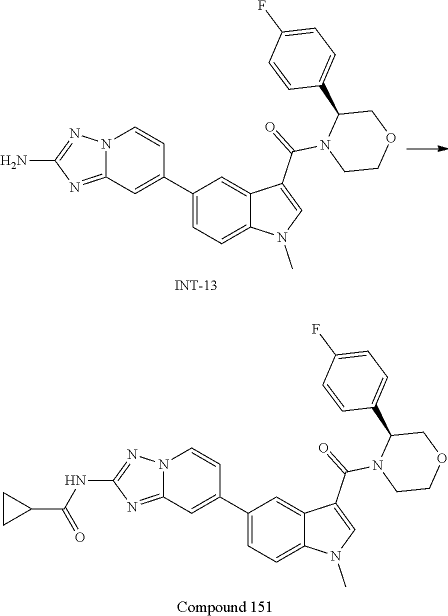 [SMILES: Cn1cc(C(=O)N2CCOC[C@@H]2c2ccc(F)cc2)c2cc(-c3ccn4nc(N)nc4c3)ccc21 ; "INT-13" ; Cn1cc(C(=O)N2CCOC[C@@H]2c2ccc(F)cc2)c2cc(-c3ccn4nc(NC(=O)C5CC5)nc4c3)ccc21]

### Preparation of Compound 151

INT-13 (94 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 151 with a yield of 56%. MS (ESI, positive ion) m/z: 539.2 [M+H]⁺.

¹H NMR (400 MHz, DMSO-d₆) δ 10.46 (s, 1H), 8.92 (d, J=7.3 Hz, 1H), 8.22-8.11 (m, 1H), 7.97-7.88 (m, 2H), 7.76 (dd, J=8.7, 1.8 Hz, 1H), 7.67 (d, J=8.6 Hz, 1H), 7.53 (dt, J=9.5, 4.7 Hz, 2H), 7.48 (dd, J=7.2, 2.0 Hz, 1H), 7.24 (t, J=8.9 Hz, 2H), 5.63 (s, 1H), 4.49 (d, J=12.3 Hz, 1H), 3.94 (dd, J=12.2, 3.5 Hz, 2H), 3.89 (s, 3H), 3.29 (d, J=12.6 Hz, 1H), 1.94-1.80 (m, 2H). 1.35 (m, 5H).

### Example 152: Preparation of Compound 152

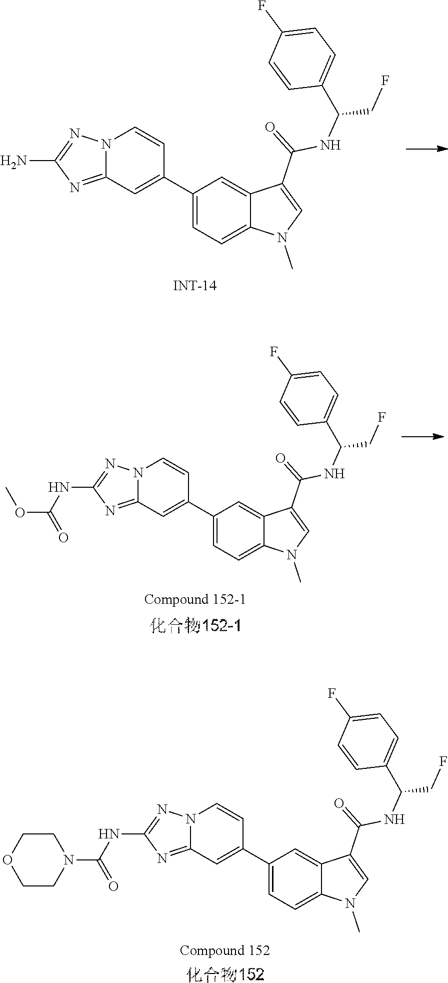 [SMILES: Cn1cc(C(=O)N[C@@H](CF)c2ccc(F)cc2)c2cc(-c3ccn4nc(N)nc4c3)ccc21 ; "INT-14" ; COC(=O)Nc1nc2cc(-c3ccc4c(c3)c(C(=O)N[C@@H](CF)c3ccc(F)cc3)cn4C)ccn2n1 ; Cn1cc(C(=O)N[C@@H](CF)c2ccc(F)cc2)c2cc(-c3ccn4nc(NC(=O)N5CCOCC5)nc4c3)ccc21]

### Preparation of Compound 152-1

Intermediate INT-14 (44.6 g, 100 mmol) was dissolved in dichloromethane (1 L), methyl chloroformate (11.2 g, 120 mmol) and N,N-diisopropylethylamine (25.8 g, 200 mmol) were added, and the mixture was stirred at room temperature for 2 h. Petroleum ether (3 L) was added to the reaction mixture for crystallization; the solid was filtered with suction, washed with water, and dried to obtain Compound 152-1 with a yield of 71%. MS (ESI, positive ion) m/z: 505.2 [M+H]⁺.

### Preparation of Compound 152

Compound 152-1 (100 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 152 with a yield of 64%. MS (ESI, positive ion) m/z: 560.2 [M+H]⁺.

¹H NMR (400 MHz, DMSO-d₆) δ 9.66 (s, 1H), 9.21 (d, J=8.6 Hz, 1H), 8.86 (d, J=7.1 Hz, 1H), 8.52 (s, 1H), 8.03-7.97 (m, 1H), 7.98-7.90 (m, 2H), 7.63-7.54 (m, 2H), 7.49-7.42 (m, 1H), 7.22 (t, J=8.9 Hz, 2H), 5.50 (dq, J=13.5, 8.4, 6.8 Hz, 1H), 5.01-4.73 (m, 1H), 4.69 (ddd, J=46.3, 9.5, 5.6 Hz, 1H), 4.24 (s, 3H), 3.66-3.59 (m, 4H), 3.47 (t, J=4.7 Hz, 4H).

### Example 153: Preparation of Compound 153

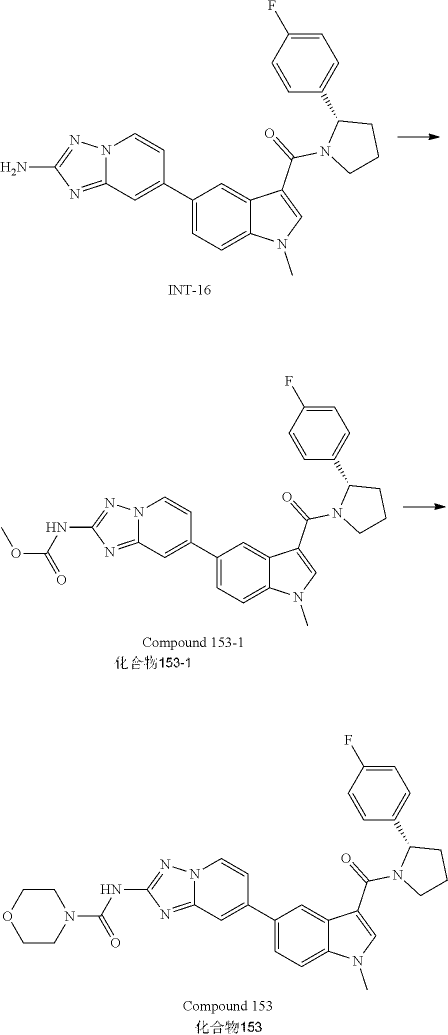 [SMILES: Cn1cc(C(=O)N2CCC[C@H]2c2ccc(F)cc2)c2cc(-c3ccn4nc(N)nc4c3)ccc21 ; "INT-16" ; COC(=O)Nc1nc2cc(-c3ccc4c(c3)c(C(=O)N3CCC[C@H]3c3ccc(F)cc3)cn4C)ccn2n1 ; "Compound 153-1" ; Cn1cc(C(=O)N2CCC[C@H]2c2ccc(F)cc2)c2cc(-c3ccn4nc(NC(=O)N5CCOCC5)nc4c3)ccc21 ; "Compound 153"]

### Preparation of Compound 153-1

Intermediate INT-16 (4.5 g, 10 mmol) was dissolved in dichloromethane (0.3 L), methyl chloroformate (1.12 g, 12 mmol) and N,N-diisopropylethylamine (2.58 g, 20 mmol) were added, and the mixture was stirred at room temperature for 2 h. Petroleum ether (3 L) was added to the reaction mixture for crystallization; the solid was filtered with suction, washed with water, and dried to obtain Compound 153-1 with a yield of 71%. MS (ESI, positive ion) m/z: 513.2 [M+H]⁺.

### Preparation of Compound 153

Compound 152-1 (100 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 153 with a yield of 64%. MS (ESI, positive ion) m/z: 568.2 [M+H]⁺.

¹H NMR (400 MHz, DMSO-d6) δ 11.07 (s, 1H), 8.82 (m, 1H), 8.48 (d, J=1.8 Hz, 1H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (m, 1H), 7.13 (t, J=8.7 Hz, 2H), 4.71-4.52 (m, 2H), 3.74 (s, 3H), 3.55-3.31 (m, 10H), 2.10-1.74 (m, 4H).

### Example 154: Preparation of Compound 154

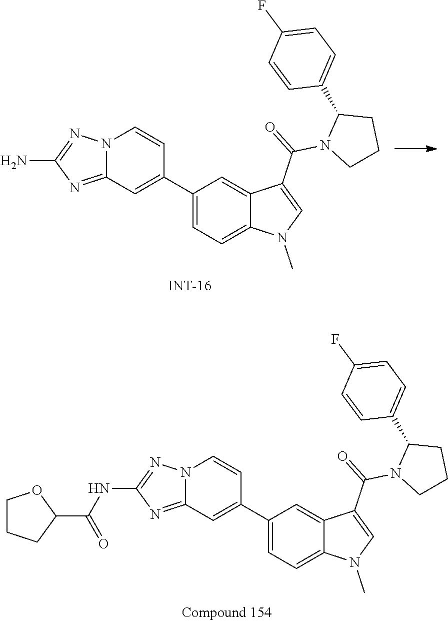 [SMILES: Cn1cc(C(=O)N2CCC[C@H]2c2ccc(F)cc2)c2cc(-c3ccn4nc(N)nc4c3)ccc21 ; "INT-16" ; Cn1cc(C(=O)N2CCC[C@H]2c2ccc(F)cc2)c2cc(-c3ccn4nc(NC(=O)C5CCCO5)nc4c3)ccc21]

### Preparation of Compound 154

INT-16 (90 mg, 0.2 mmol) was dissolved in pyridine (1 mL); tetrahydrofuran-2-carboxylic acid (35 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 154 with a yield of 78%. MS (ESI, positive ion) m/z: 553.2 [M+H]⁺.

¹H NMR (400 MHz, DMSO-d6) δ 11.07 (s, 1H), 8.82 (m, 1H), 8.48 (d, J=1.8 Hz, 1H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (m, 1H), 7.13 (t, J=8.7 Hz, 2H), 4.71-4.52 (m, 2H), 3.86-3.70 (m, 5H), 3.51-3.40 (m, 2H), 2.10-1.74 (m, 6H).

### Example 155: Preparation of Compound 155

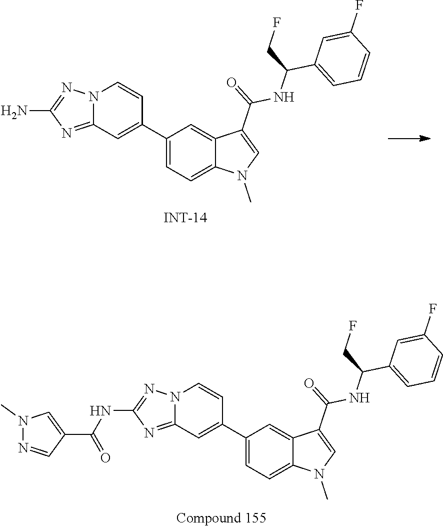 [SMILES: Cn1cc(C(=O)N[C@@H](CF)c2cccc(F)c2)c2cc(-c3ccn4nc(N)nc4c3)ccc21 ; "INT-14" ; Cn1cc(C(=O)Nc2nc3cc(-c4ccc5c(c4)c(C(=O)N[C@@H](CF)c4cccc(F)c4)cn5C)ccn3n2)cn1]

### Preparation of Compound 155

Intermediate INT-14 (86 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 155 with a yield of 45%. MS (ESI, positive ion) m/z: 555.2 [M+H]⁺.

¹H NMR (400 MHz, DMSO-d₆) δ 10.88 (s, 1H), 8.85 (d, J=7.2 Hz, 1H), 8.61 (d, J=8.3 Hz, 1H), 8.50 (d, J=1.9 Hz, 1H), 8.36 (s, 1H), 8.25 (s, 1H), 8.06 (s, 1H), 7.86 (s, 1H), 7.70 (d, J=1.8 Hz, 1H), 7.64 (d, J=8.7 Hz, 1H), 7.45-7.35 (m, 2H), 7.33-7.26 (m, 2H), 7.09 (t, J=8.6 Hz, 1H), 5.44 (dq, J=15.0, 7.0 Hz, 1H), 4.71 (d, J=6.5 Hz, 1H), 4.60 (d, J=6.4 Hz, 1H), 3.87 (s, 3H), 3.85 (s, 3H).

### Example 156: Preparation of Compound 156

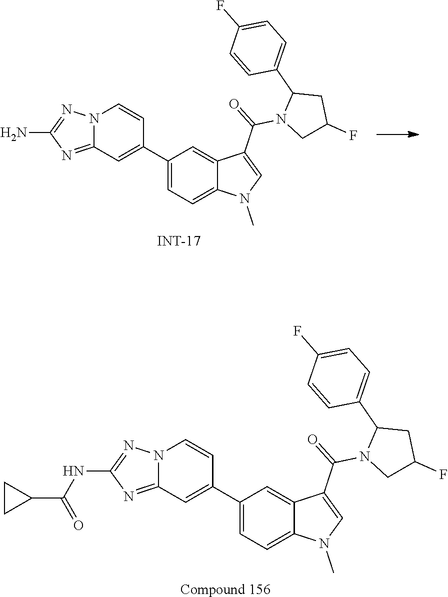 [SMILES: Cn1cc(C(=O)N2CC(F)CC2c2ccc(F)cc2)c2cc(-c3ccn4nc(N)nc4c3)ccc21 ; "INT-17" ; Cn1cc(C(=O)N2CC(F)CC2c2ccc(F)cc2)c2cc(-c3ccn4nc(NC(=O)C5CC5)nc4c3)ccc21 ; "Compound 156"]

### Preparation of Compound 156

INT-17 (94 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 156 with a yield of 66%. MS (ESI, positive ion) m/z: 541.2 [M+H]⁺.

### Example 157: Preparation of Compound 157

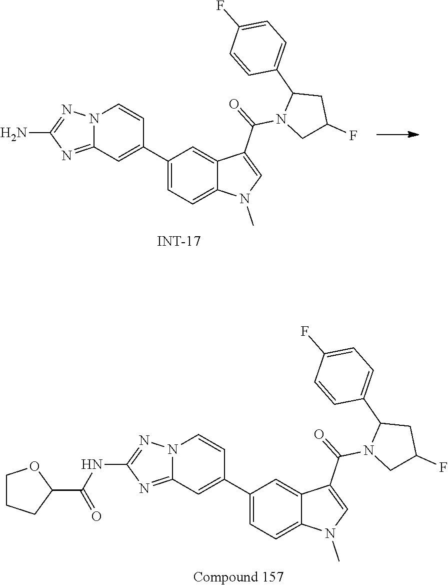 [SMILES: Cn1cc(C(=O)N2CC(F)CC2c2ccc(F)cc2)c2cc(-c3ccn4nc(N)nc4c3)ccc21 ; "INT-17" ; Cn1cc(C(=O)N2CC(F)CC2c2ccc(F)cc2)c2cc(-c3ccn4nc(NC(=O)C5CCCO5)nc4c3)ccc21 ; "Compound 157"]

### Preparation of Compound 157

INT-17 (90 mg, 0.2 mmol) was dissolved in pyridine (1 mL); tetrahydrofuran-2-carboxylic acid (35 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 157 with a yield of 78%. MS (ESI, positive ion) m/z: 570.2 [M+H]⁺.

¹H NMR (400 MHz, DMSO-d6) δ 11.07 (s, 1H), 8.82 (m, 1H), 8.35 (s, 1H), 7.85 (m, 2H), 7.73 (d, J=8.6 Hz, 1H), 7.59-7.49 (m, 2H), 7.42 (dd, J=7.1, 2.0 Hz, 1H), 7.32 (dd, J=8.5, 5.5 Hz, 2H), 7.13 (t, J=8.7 Hz, 2H), 4.81-4.75 (m, 1H), 3.8-3.75 (m, 7H), 2.51-1.83 (m, 4H).

### Example 158: Preparation of Compound 158

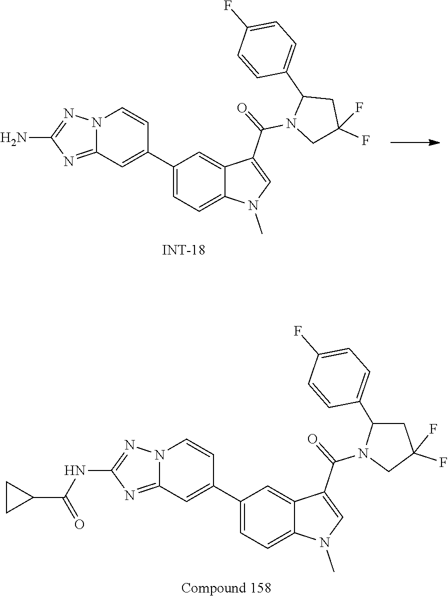 [SMILES: Cn1cc(C(=O)N2CC(F)(F)CC2c2ccc(F)cc2)c2cc(-c3ccn4nc(N)nc4c3)ccc21 ; "INT-18" ; Cn1cc(C(=O)N2CC(F)(F)CC2c2ccc(F)cc2)c2cc(-c3ccn4nc(NC(=O)C5CC5)nc4c3)ccc21 ; "Compound 158"]

### Preparation of Compound 158

INT-18 (98 mg, 0.2 mmol) was dissolved in pyridine (1 mL), cyclopropanecarboxylic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 158 with a yield of 56%. MS (ESI, positive ion) m/z: 559.2 [M+H]⁺.

¹H NMR (400 MHz, DMSO-d6) δ 11.07 (s, 1H), 8.82 (m, 1H), 8.35 (s, 1H), 7.85 (m, 2H), 7.73 (d, J=8.6 Hz, 1H), 7.59-7.49 (m, 2H), 7.42 (dd, J=7.1, 2.0 Hz, 1H), 7.32 (dd, J=8.5, 5.5 Hz, 2H), 7.13 (t, J=8.7 Hz, 2H), 4.81-4.75 (m, 1H), 3.8-3.75 (s, 5H), 2.51-2.42 (m, 2H), 1.51-1.43 (m, 1H), 1.12-1.06 (m, 4H).

### Example 159: Preparation of Compound 159

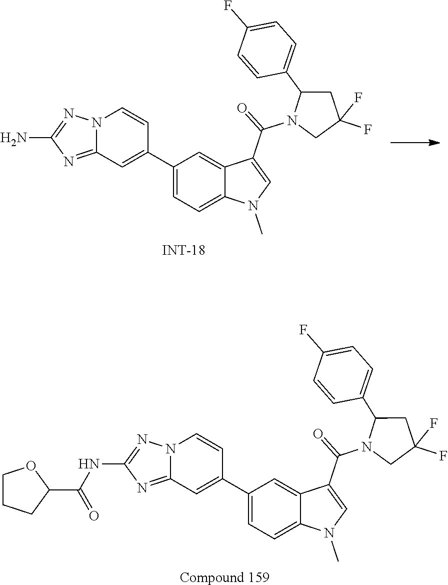 [SMILES: Cn1cc(C(=O)N2CC(F)(F)CC2c2ccc(F)cc2)c2cc(-c3ccn4nc(N)nc4c3)ccc21 ; "INT-18" ; Cn1cc(C(=O)N2CC(F)(F)CC2c2ccc(F)cc2)c2cc(-c3ccn4nc(NC(=O)C5CCCO5)nc4c3)ccc21 ; "Compound 159"]

### Preparation of Compound 159

INT-18 (90 mg, 0.2 mmol) was dissolved in pyridine (1 mL); tetrahydrofuran-2-carboxylic acid (35 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 159 with a yield of 72%. MS (ESI, positive ion) m/z: 589.2 [M+H]⁺.

¹H NMR (400 MHz, DMSO-d6) δ 11.07 (s, 1H), 8.82 (m, 1H), 8.35 (s, 1H), 7.85 (m, 2H), 7.73 (d, J=8.6 Hz, 1H), 7.59-7.49 (m, 2H), 7.42 (dd, J=7.1, 2.0 Hz, 1H), 7.32 (dd, J=8.5, 5.5 Hz, 2H), 7.13 (t, J=8.7 Hz, 2H), 4.81-4.75 (m, 1H), 3.8-3.75 (m, 7H), 2.51-1.83 (m, 4H).

### Example 160: Preparation of Compound 160

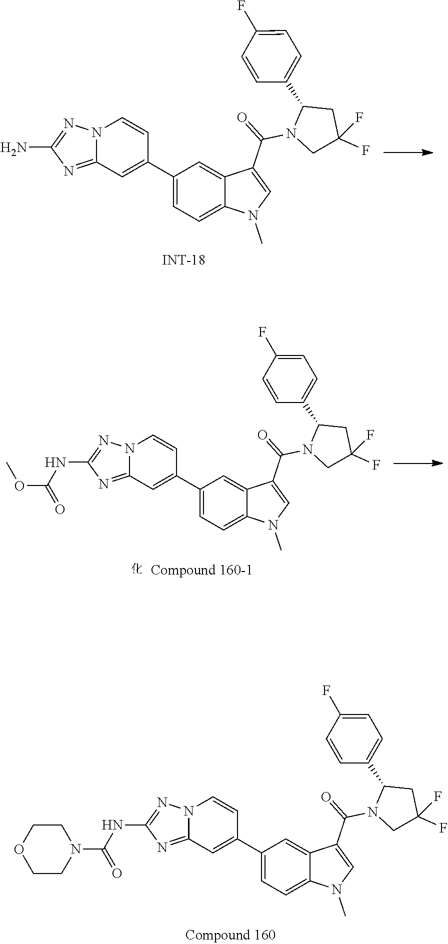 [SMILES: Cn1cc(C(=O)N2CC(F)(F)C[C@H]2c2ccc(F)cc2)c2cc(-c3ccn4nc(N)nc4c3)ccc21 ; "INT-18" ; COC(=O)Nc1nc2cc(-c3ccc4c(c3)c(C(=O)N3CC(F)(F)C[C@H]3c3ccc(F)cc3)cn4C)ccn2n1 ; Cn1cc(C(=O)N2CC(F)(F)C[C@H]2c2ccc(F)cc2)c2cc(-c3ccn4nc(NC(=O)N5CCOCC5)nc4c3)ccc21]

### Preparation of Compound 160-1

Intermediate INT-18 (490 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 160-1 with a yield of 61%. MS (ESI, positive ion) m/z: 549.2 [M+H]⁺.

### Preparation of Compound 160

Compound 160-1 (110 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 160 with a yield of 68%. MS (ESI, positive ion) m/z: 604.2 [M+H]⁺.

¹H NMR (400 MHz, DMSO-d6) δ 11.07 (s, 1H), 8.82 (m, 1H), 8.35 (s, 1H), 7.85 (m, 2H), 7.73 (d, J=8.6 Hz, 1H), 7.59-7.49 (m, 2H), 7.42 (dd, J=7.1, 2.0 Hz, 1H), 7.32 (dd, J=8.5, 5.5 Hz, 2H), 7.13 (t, J=8.7 Hz, 2H), 4.82-4.70 (m, 1H), 3.89-3.71 (m, 3H), 3.64 (m, 4H), 3.31 (m, 4H), 2.52-2.28 (m, 2H).

### Example 161: Preparation of Compound 161

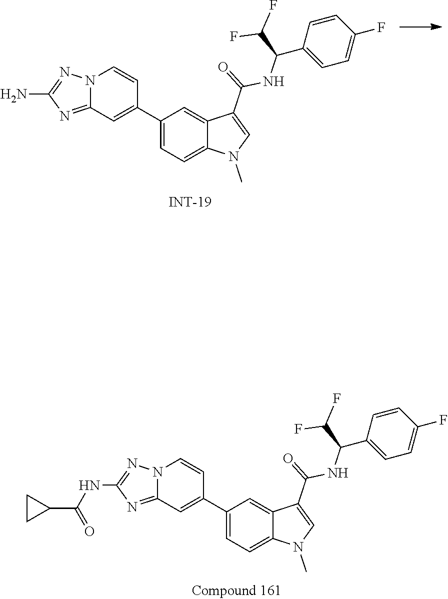

### Preparation of Compound 161

Intermediate INT-19 (93 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 161 with a yield of 65%. MS (ESI, positive ion) m/z: 533.2 [M+H]⁺.

¹H NMR (400 MHz, DMSO-d₆) δ 11.28 (s, 1H), 8.92 (d, J=7.2 Hz, 1H), 8.78 (d, J=9.1 Hz, 1H), 8.55 (d, J=1.9 Hz, 1H), 8.36 (s, 1H), 7.94 (d, J=1.9 Hz, 1H), 7.77 (dd, J=8.7, 1.9 Hz, 1H), 7.70 (d, J=8.7 Hz, 1H), 7.66-7.62 (m, 2H), 7.50 (dd, J=7.1, 2.0 Hz, 1H), 7.27 (t, J=8.9 Hz, 2H), 6.37-6.12 (m, 2H), 4.02 (d, J=8.5 Hz, 5H), 3.85 (q, J=7.0 Hz, 1H), 2.32-2.19 (m, 1H), 2.07-1.84 (m, 3H), 1.50 (d, J=7.0 Hz, 3H).

### Example 162: Preparation of Compound 162

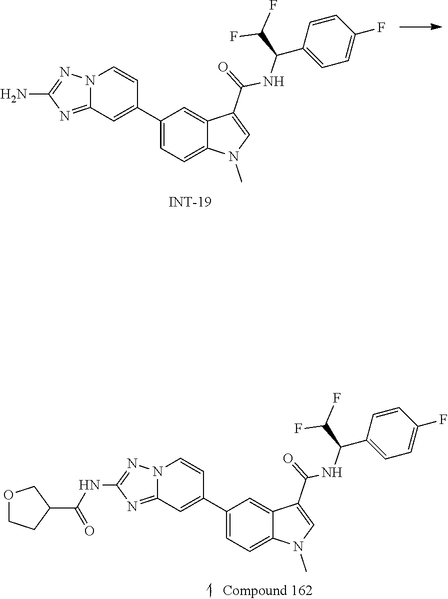

### Preparation of Compound 162

Intermediate INT-19 (93 mg, 0.2 mmol) was dissolved in pyridine (1 mL); 3-tetrahydrofuroic acid (30 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 162 with a yield of 55%. MS (ESI, positive ion) m/z: 564.2 [M+H]⁺.

¹H NMR (400 MHz, DMSO-d₆) δ 11.28 (s, 1H), 8.92 (d, J=7.2 Hz, 1H), 8.78 (d, J=9.1 Hz, 1H), 8.55 (d, J=1.9 Hz, 1H), 8.36 (s, 1H), 7.94 (d, J=1.9 Hz, 1H), 7.77 (dd, J=8.7, 1.9 Hz, 1H), 7.70 (d, J=8.7 Hz, 1H), 7.66-7.62 (m, 2H), 7.50 (dd, J=7.1, 2.0 Hz, 1H), 7.27 (t, J=8.9 Hz, 2H), 6.37-6.12 (m, 2H), 4.12-3.75 (m, 7H), 2.72-2.63 (m, 1H), 2.17-1.94 (m, 2H).

### Example 163: Preparation of Compound 163

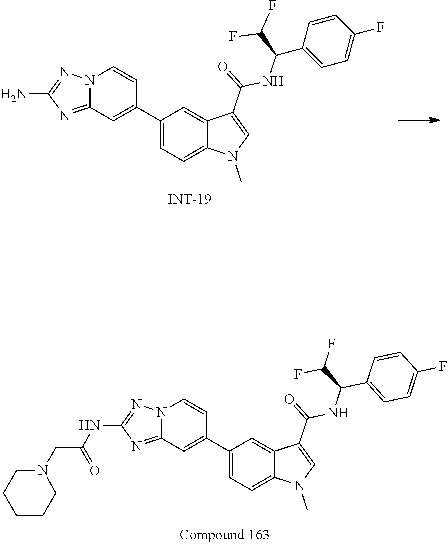 [SMILES: Cn1cc(C(=O)N[C@H](c2ccc(F)cc2)C(F)F)c2cc(-c3ccn4nc(N)nc4c3)ccc21 ; "INT-19" ; Cn1cc(C(=O)N[C@H](c2ccc(F)cc2)C(F)F)c2cc(-c3ccn4nc(NC(=O)CN5CCCCC5)nc4c3)ccc21]

### Preparation of Compound 163

Intermediate INT-19 (93 mg, 0.2 mmol) was dissolved in pyridine (1 mL); 1-piperidineacetic acid (42 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 163 with a yield of 55%. MS (ESI, positive ion) m/z: 590.2 [M+H]⁺.

¹H NMR (400 MHz, DMSO-d₆) δ 11.28 (s, 1H), 8.92 (d, J=7.2 Hz, 1H), 8.78 (d, J=9.1 Hz, 1H), 8.55 (d, J=1.9 Hz, 1H), 8.36 (s, 1H), 7.94 (d, J=1.9 Hz, 1H), 7.77 (dd, J=8.7, 1.9 Hz, 1H), 7.70 (d, J=8.7 Hz, 1H), 7.66-7.62 (m, 2H), 7.50 (dd, J=7.1, 2.0 Hz, 1H), 7.27 (t, J=8.9 Hz, 2H), 6.37-6.12 (m, 2H), 3.81 (s, 3H), 3.35 (s, 2H), 2.51-3.36 (m, 4H), 1.62-1.42 (m, 6H).

### Example 164: Preparation of Compound 164

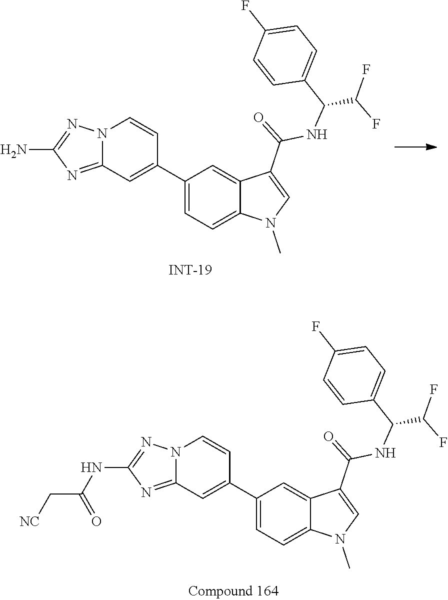 [SMILES: Cn1cc(C(=O)N[C@H](c2ccc(F)cc2)C(F)F)c2cc(-c3ccn4nc(N)nc4c3)ccc21 ; "INT-19" ; Cn1cc(C(=O)N[C@H](c2ccc(F)cc2)C(F)F)c2cc(-c3ccn4nc(NC(=O)CC#N)nc4c3)ccc21]

### Preparation of Compound 164

INT-19 (93 mg, 0.2 mmol) was dissolved in pyridine (1 mL), cyanoacetic acid (29 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 164 with a yield of 58%. MS (ESI, positive ion) m/z: 532.2 [M+H]⁺.

¹H NMR (400 MHz, DMSO-d₆) δ 11.28 (s, 1H), 8.92 (d, J=7.2 Hz, 1H), 8.78 (d, J=9.1 Hz, 1H), 8.55 (d, J=1.9 Hz, 1H), 8.36 (s, 1H), 7.94 (d, J=1.9 Hz, 1H), 7.77 (dd, J=8.7, 1.9 Hz, 1H), 7.70 (d, J=8.7 Hz, 1H), 7.66-7.62 (m, 2H), 7.50 (dd, J=7.1, 2.0 Hz, 1H), 7.27 (t, J=8.9 Hz, 2H), 6.37 (td, J=55.5, 4.2 Hz, 1H), 5.61 (dt, J=14.6, 9.8 Hz, 1H), 4.06 (s, 2H), 3.94 (s, 3H).

### Example 165: Preparation of Compound 165

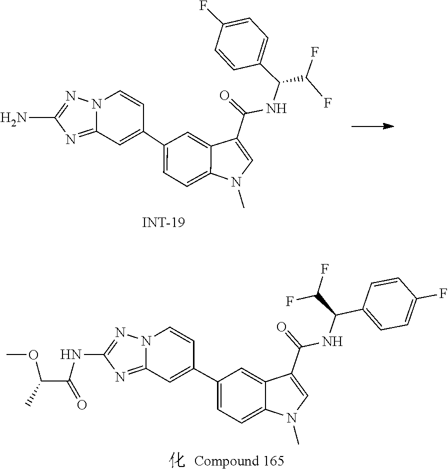 [SMILES: Cn1cc(C(=O)N[C@H](c2ccc(F)cc2)C(F)F)c2cc(-c3ccn4nc(N)nc4c3)ccc21 ; "INT-19" ; CO[C@@H](C)C(=O)Nc1nc2cc(-c3ccc4c(c3)c(C(=O)N[C@H](c3ccc(F)cc3)C(F)F)cn4C)ccn2n1 ; "Compound 165"]

### Preparation of Compound 165

INT-19 (93 mg, 0.2 mmol) was dissolved in pyridine (1 mL), (S)-2-methoxypropanoic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 165 with a yield of 62%. MS (ESI, positive ion) m/z: 551.2 [M+H]⁺.

¹H NMR (400 MHz, DMSO-d₆) δ 10.72 (s, 1H), 8.91 (d, J=7.1 Hz, 1H), 8.78 (d, J=9.0 Hz, 1H), 8.55 (d, J=1.8 Hz, 1H), 8.36 (s, 1H), 7.92 (d, J=2.0 Hz, 1H), 7.77 (dd, J=8.7, 1.9 Hz, 1H), 7.74-7.60 (m, 3H), 7.49 (dd, J=7.2, 2.0 Hz, 1H), 7.27 (t, J=8.9 Hz, 2H), 6.37 (td, J=55.6, 4.1 Hz, 1H), 5.66-5.58 (m, 1H), 4.05 (s, 1H), 3.94 (s, 3H), 3.32 (s, 3H), 1.34 (d, J=6.7 Hz, 3H).

### Example 166: Preparation of Compound 166

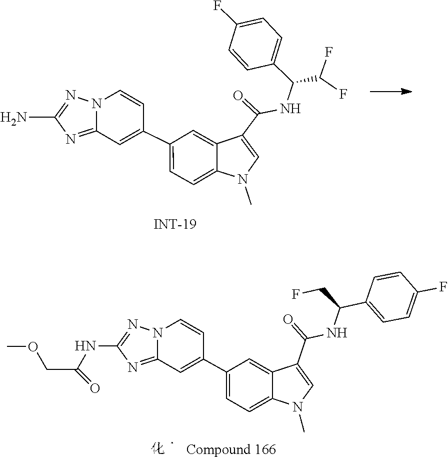 [SMILES: Cn1cc(C(=O)N[C@H](c2ccc(F)cc2)C(F)F)c2cc(-c3ccn4nc(N)nc4c3)ccc21 ; "INT-19" ; COCC(=O)Nc1nc2cc(-c3ccc4c(c3)c(C(=O)N[C@@H](CF)c3ccc(F)cc3)cn4C)ccn2n1 ; "Compound 166"]

### Preparation of Compound 166

INT-19 (93 mg, 0.2 mmol) was dissolved in pyridine (1 mL), 2-methoxyacetic acid (26 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 166 with a yield of 69%. MS (ESI, positive ion) m/z: 537.2 [M+H]⁺.

¹H NMR (400 MHz, DMSO-d₆) δ 10.68 (s, 1H), 8.90 (d, J=7.1 Hz, 1H), 8.77 (d, J=9.1 Hz, 1H), 8.55 (d, J=1.8 Hz, 1H), 8.36 (s, 1H), 7.92 (d, J=1.9 Hz, 1H), 7.77 (dd, J=8.5, 1.9 Hz, 1H), 7.70 (d, J=8.6 Hz, 1H), 7.64 (dd, J=8.5, 5.5 Hz, 2H), 7.49 (dd, J=7.2, 1.9 Hz, 1H), 7.27 (t, J=8.8 Hz, 2H), 6.37 (td, J=55.5, 4.1 Hz, 1H), 5.66-5.58 (m, 1H), 4.18 (s, 2H), 3.94 (s, 3H), 3.39 (s, 3H).

### Example 167: Preparation of Compound 167

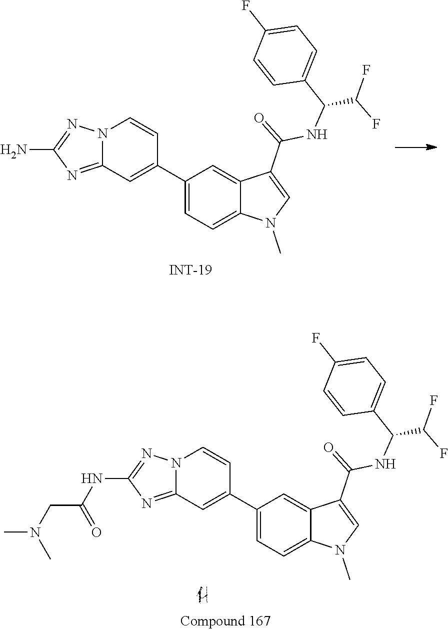 [SMILES: Cn1cc(C(=O)N[C@H](c2ccc(F)cc2)C(F)F)c2cc(-c3ccn4nc(N)nc4c3)ccc21 ; "INT-19" ; CN(C)CC(=O)Nc1nc2cc(-c3ccc4c(c3)c(C(=O)N[C@H](c3ccc(F)cc3)C(F)F)cn4C)ccn2n1]

### Preparation of Compound 167

INT-19 (93 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 167 with a yield of 73%. MS (ESI, positive ion) m/z: 550.2 [M+H]⁺.

¹H NMR (400 MHz, DMSO-d₆) δ 10.45 (s, 1H), 8.89 (d, J=7.2 Hz, 1H), 8.76 (d, J=9.0 Hz, 1H), 8.53 (s, 1H), 8.35 (s, 1H), 7.90 (s, 1H), 7.76 (d, J=8.8 Hz, 1H), 7.72-7.59 (m, 3H), 7.47 (d, J=7.1 Hz, 1H), 7.26 (t, J=8.7 Hz, 2H), 6.53-6.19 (m, 1H), 5.61 (d, J=12.9 Hz, 1H), 3.93 (s, 3H), 3.20 (s, 2H), 2.31 (s, 6H).

### Example 168: Preparation of Compound 168

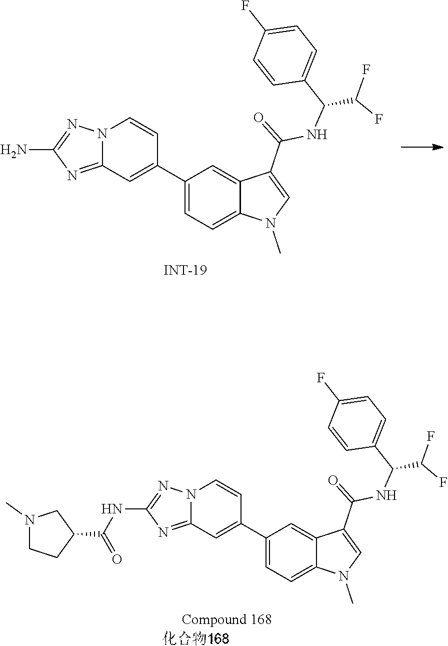 [SMILES: Cn1cc(C(=O)N[C@H](c2ccc(F)cc2)C(F)F)c2cc(-c3ccn4nc(N)nc4c3)ccc21 ; "INT-19" ; CN1CC[C@@H](C(=O)Nc2nc3cc(-c4ccc5c(c4)c(C(=O)N[C@H](c4ccc(F)cc4)C(F)F)cn5C)ccn3n2)C1]

### Preparation of Compound 168

INT-19 (93 mg, 0.2 mmol) was dissolved in pyridine (1 mL), (R)-1-methylpyrrolidine-3-carboxylic acid (39 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 168 with a yield of 68%. MS (ESI, positive ion) m/z: 576.2 [M+H]⁺.

¹H NMR (400 MHz, DMSO-d₆) δ 10.91 (s, 1H), 8.89 (d, J=7.1 Hz, 1H), 8.78 (d, J=9.0 Hz, 1H), 8.55 (s, 1H), 8.36 (s, 1H), 7.90 (d, J=1.9 Hz, 1H), 7.77 (dd, J=8.7, 1.9 Hz, 1H), 7.70 (d, J=8.6 Hz, 1H), 7.64 (dd, J=8.4, 5.4 Hz, 2H), 7.48 (dd, J=7.1, 2.0 Hz, 1H), 7.27 (t, J=8.7 Hz, 2H), 6.55-6.19 (m, 1H), 5.65-5.58 (m, 1H), 3.94 (s, 3H), 2.82 (dt, J=8.1, 5.0 Hz, 2H), 2.45 (s, 3H), 2.36 (s, 1H), 2.07 (ddd, J=20.3, 16.3, 9.0 Hz, 2H), 1.37 (d, J=6.1 Hz, 2H).

### Example 169: Preparation of Compound 169

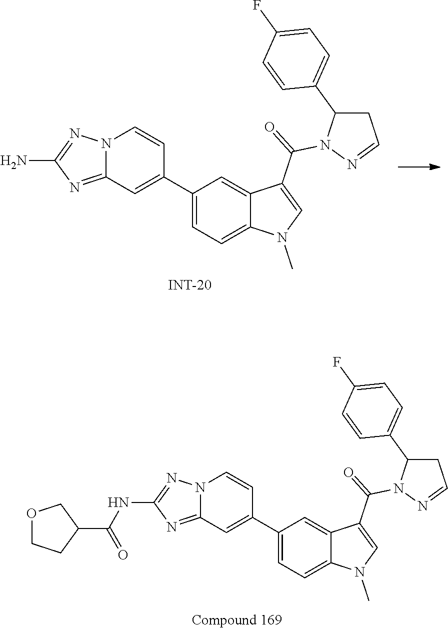 [SMILES: Cn1cc(C(=O)N2N=CCC2c2ccc(F)cc2)c2cc(-c3ccn4nc(N)nc4c3)ccc21 ; "INT-20" ; Cn1cc(C(=O)N2N=CCC2c2ccc(F)cc2)c2cc(-c3ccn4nc(NC(=O)C5CCOC5)nc4c3)ccc21]

### Preparation of Compound 162

Intermediate INT-20 (90 mg, 0.2 mmol) was dissolved in pyridine (1 mL); 3-tetrahydrofuroic acid (30 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 169 with a yield of 55%. MS (ESI, positive ion) m/z: 552.2 [M+H]⁺.

¹H NMR (400 MHz, DMSO-d6) δ 11.07 (s, 1H), 8.82 (m, 2H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (dd, J=8.5, 5.5 Hz, 2H), 7.13 (t, J=8.7 Hz, 2H), 4.02 (d, J=8.5 Hz, 5H), 3.85 (q, J=7.0 Hz, 1H), 2.32-2.19 (m, 1H), 2.07-1.84 (m, 3H), 1.50 (d, J=7.0 Hz, 3H).

### Example 170: Preparation of Compound 170

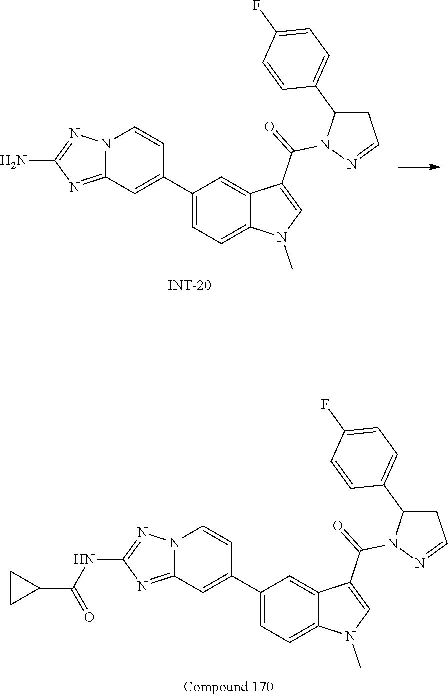 [SMILES: Cn1cc(C(=O)N2N=CCC2c2ccc(F)cc2)c2cc(-c3ccn4nc(N)nc4c3)ccc21 ; "INT-20" ; Cn1cc(C(=O)N2N=CCC2c2ccc(F)cc2)c2cc(-c3ccn4nc(NC(=O)C5CC5)nc4c3)ccc21]

### Preparation of Compound 170

Intermediate INT-20 (93 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 170 with a yield of 61%. MS (ESI, positive ion) m/z: 522.2 [M+H]⁺.

¹H NMR (400 MHz, DMSO-d6) δ 11.07 (s, 1H), 8.82 (m, 2H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (dd, J=8.5, 5.5 Hz, 2H), 7.13 (t, J=8.7 Hz, 2H), 4.82-4.70 (m, 1H), 3.83 (s, 3H), 2.81-2.56 (m, 2H), 1.45-1.38 (m, 1H), 1.12-0.96 (m, 4H).

### Example 171: Preparation of Compound 171

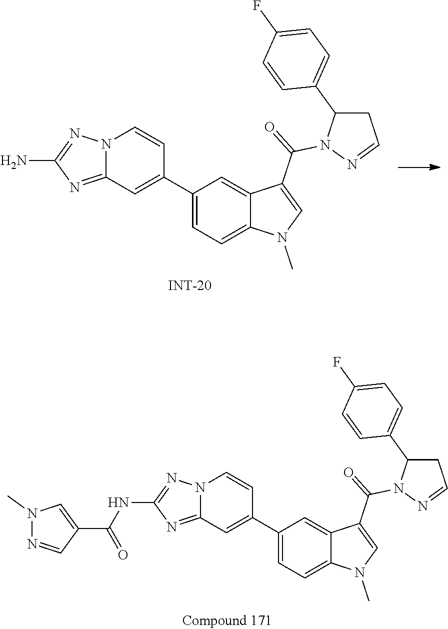 [SMILES: Cn1cc(C(=O)N2N=CCC2c2ccc(F)cc2)c2cc(-c3ccn4nc(N)nc4c3)ccc21 ; "INT-20" ; Cn1cc(C(=O)Nc2nc3cc(-c4ccc5c(c4)c(C(=O)N4N=CCC4c4ccc(F)cc4)cn5C)ccn3n2)cn1]

### Preparation of Compound 171

INT-20 (94 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 171 with a yield of 62%. MS (ESI, positive ion) m/z: 562.2 [M+H]⁺.

¹H NMR (400 MHz, DMSO-d6) δ 11.07 (s, 1H), 8.82 (m, 2H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (dd, J=8.5, 5.5 Hz, 2H), 7.13 (t, J=8.7 Hz, 2H), 4.82-4.70 (m, 1H), 3.95 (s, 3H), 3.73 (s, 3H), 2.81-2.56 (m, 2H).

### Example 172: Preparation of Compound 172

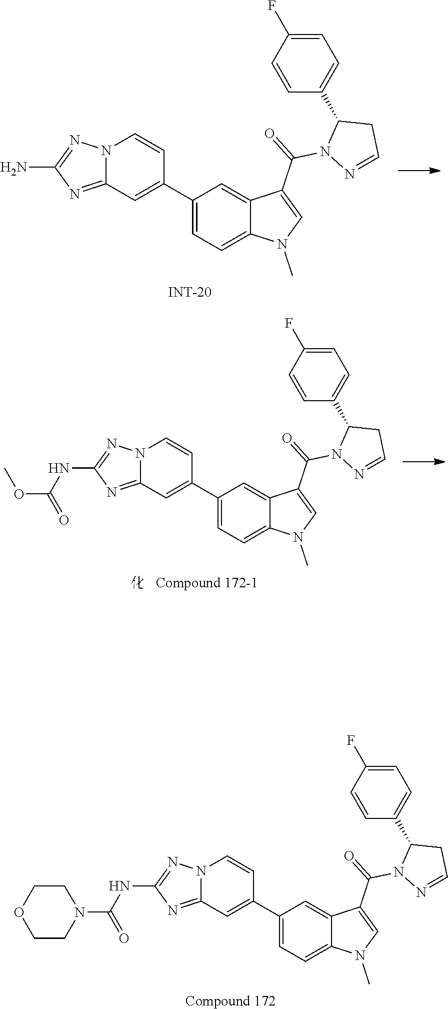 [SMILES: Cn1cc(C(=O)N2N=CC[C@H]2c2ccc(F)cc2)c2cc(-c3ccn4nc(N)nc4c3)ccc21 ; "INT-20" ; COC(=O)Nc1nc2cc(-c3ccc4c(c3)c(C(=O)N3N=CC[C@H]3c3ccc(F)cc3)cn4C)ccn2n1 ; "Compound 172-1" ; Cn1cc(C(=O)N2N=CC[C@H]2c2ccc(F)cc2)c2cc(-c3ccn4nc(NC(=O)N5CCOCC5)nc4c3)ccc21 ; "Compound 172"]

### Preparation of Compound 172-1

Intermediate INT-20 (490 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 172-1 with a yield of 61%. MS (ESI, positive ion) m/z: 512.2 [M+H]⁺.

### Preparation of Compound 172

Compound 172-1 (102 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 172 with a yield of 68%. MS (ESI, positive ion) m/z: 567.2 [M+H]⁺.

¹H NMR (400 MHz, DMSO-d6) δ 11.07 (s, 1H), 8.82 (m, 2H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (dd, J=8.5, 5.5 Hz, 2H), 7.13 (t, J=8.7 Hz, 2H), 4.82-4.70 (m, 1H), 3.83 (s, 3H), 3.64 (m, 4H), 3.31 (m, 4H), 2.81-2.56 (m, 2H).

### Example 173: Preparation of Compound 173

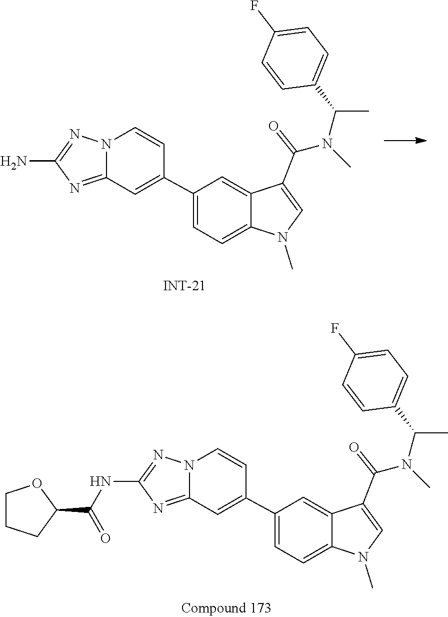 [SMILES: C[C@@H](c1ccc(F)cc1)N(C)C(=O)c1cn(C)c2ccc(-c3ccn4nc(N)nc4c3)cc12 ; "INT-21" ; C[C@@H](c1ccc(F)cc1)N(C)C(=O)c1cn(C)c2ccc(-c3ccn4nc(NC(=O)[C@H]5CCCO5)nc4c3)cc12]

### Preparation of Compound 173

INT-21 (88 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 173 with a yield of 71%. MS (ESI, positive ion) m/z: 543.2 [M+H]⁺.

¹H NMR (400 MHz, DMSO-d₆) δ 10.47 (s, 1H), 8.89 (d, J=7.1 Hz, 1H), 8.29 (d, J=1.8 Hz, 1H), 7.95-7.87 (m, 2H), 7.76 (dd, J=8.6, 1.9 Hz, 1H), 7.67 (d, J=8.6 Hz, 1H), 7.48 (dd, J=7.2, 2.0 Hz, 1H), 7.43 (dd, J=8.5, 5.5 Hz, 2H), 7.27-7.17 (m, 2H), 4.02 (q, J=7.0 Hz, 1H), 3.90 (s, 3H), 3.86 (t, J=7.0 Hz, 1H), 2.86 (s, 3H), 2.31-2.18 (m, 1H), 2.07-1.81 (m, 3H), 1.62 (d, J=7.0 Hz, 3H), 1.29 (dd, J=10.4, 6.7 Hz, 1H).

### Example 174: Preparation of Compound 174

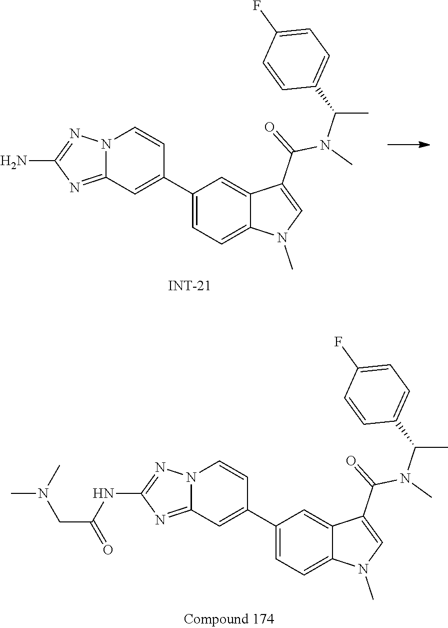

### Preparation of Compound 174

INT-21 (88 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 174 with a yield of 74%. MS (ESI, positive ion) m/z: 527.2 [M+H]⁺.

¹H NMR (400 MHz, DMSO-d₆) δ 10.44 (s, 1H), 8.88 (d, J=7.2 Hz, 1H), 8.28 (s, 1H), 7.90 (d, J=7.6 Hz, 2H), 7.79-7.72 (m, 1H), 7.67 (d, J=8.6 Hz, 1H), 7.45 (ddd, J=14.6, 7.6, 3.6 Hz, 3H), 7.22 (t, J=8.7 Hz, 2H), 3.90 (s, 3H), 3.22 (s, 2H), 2.86 (s, 3H), 2.57-2.49 (m, 6H), 1.62 (d, J=7.1 Hz, 3H).

### Example 175: Preparation of Compound 175

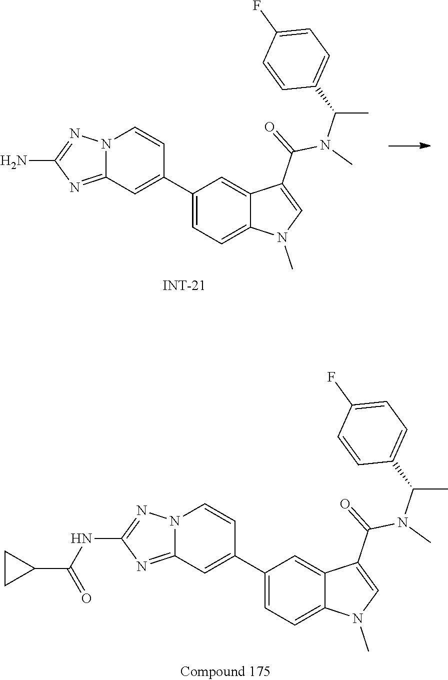 [SMILES: C[C@@H](c1ccc(F)cc1)N(C)C(=O)c1cn(C)c2ccc(-c3ccn4nc(N)nc4c3)cc12 ; "INT-21" ; C[C@@H](c1ccc(F)cc1)N(C)C(=O)c1cn(C)c2ccc(-c3ccn4nc(NC(=O)C5CC5)nc4c3)cc12]

### Preparation of Compound 175

Intermediate INT-21 (93 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 175 with a yield of 61%. MS (ESI, positive ion) m/z: 511.2 [M+H]⁺.

¹H NMR (400 MHz, DMSO-d₆) δ 11.07 (s, 1H), 8.88-8.84 (m, 1H), 8.28 (d, J=1.8 Hz, 1H), 7.91 (dd, J=3.5, 1.9 Hz, 2H), 7.77 (dd, J=8.6, 1.9 Hz, 1H), 7.68 (d, J=8.6 Hz, 1H), 7.48-7.41 (m, 3H), 7.23 (t, J=8.9 Hz, 2H), 5.88 (s, 1H), 3.90 (s, 3H), 2.86 (s, 3H), 1.63 (d, J=7.0 Hz, 3H), 0.86 (d, J=5.6 Hz, 5H).

### Example 176: Preparation of Compound 176

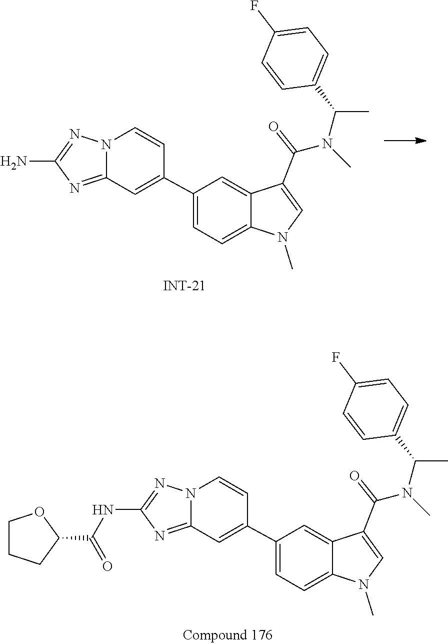 [SMILES: C[C@@H](c1ccc(F)cc1)N(C)C(=O)c1cn(C)c2ccc(-c3ccn4nc(N)nc4c3)cc12 ; "INT-21" ; C[C@@H](c1ccc(F)cc1)N(C)C(=O)c1cn(C)c2ccc(-c3ccn4nc(NC(=O)[C@@H]5CCCO5)nc4c3)cc12]

### Preparation of Compound 176

INT-21 (88 mg, 0.2 mmol) was dissolved in pyridine (1 mL), (S)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 176 with a yield of 71%. MS (ESI, positive ion) m/z: 543.2 [M+H]⁺.

¹H NMR (400 MHz, DMSO-d₆) δ 10.46 (s, 1H), 8.89 (d, J=7.1 Hz, 1H), 8.28 (d, J=1.6 Hz, 1H), 7.95-7.87 (m, 2H), 7.77 (dd, J=8.7, 1.8 Hz, 1H), 7.68 (d, J=8.6 Hz, 1H), 7.48 (dd, J=7.2, 2.0 Hz, 1H), 7.43 (dd, J=8.5, 5.5 Hz, 2H), 7.22 (t, J=8.8 Hz, 2H), 5.88 (s, 1H), 4.53 (s, 1H), 4.01 (q, J=7.0 Hz, 1H), 3.90 (s, 3H), 3.89-3.80 (m, 1H), 2.86 (s, 3H), 2.31-1.93 (m, 2H), 1.90 (dt, J=13.5, 6.6 Hz, 2H), 1.62 (d, J=7.0 Hz, 3H).

### Example 177: Preparation of Compound 177

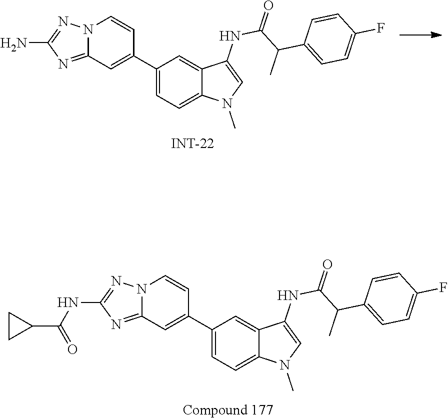 [SMILES: CC(C(=O)Nc1cn(C)c2ccc(-c3ccn4nc(N)nc4c3)cc12)c1ccc(F)cc1 ; "INT-22" ; CC(C(=O)Nc1cn(C)c2ccc(-c3ccn4nc(NC(=O)C5CC5)nc4c3)cc12)c1ccc(F)cc1]

### Preparation of Compound 177

Intermediate INT-22 (84 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 177 with a yield of 61%. MS (ESI, positive ion) m/z: 497.2 [M+H]⁺.

¹H NMR (400 MHz, DMSO-d₆) δ 11.08 (s, 1H), 10.16 (s, 1H), 8.92 (d, J=7.1 Hz, 1H), 8.37 (d, J=1.8 Hz, 1H), 7.92 (d, J=1.9 Hz, 1H), 7.86 (s, 1H), 7.72 (dd, J=8.7, 1.8 Hz, 1H), 7.56 (d, J=8.7 Hz, 1H), 7.50 (td, J=5.6, 2.3 Hz, 3H), 7.25-7.15 (m, 2H), 4.04 (q, J=7.0 Hz, 1H), 3.80 (s, 3H), 2.10 (s, 1H), 1.49 (d, J=7.0 Hz, 3H), 0.87 (d, J=6.2 Hz, 4H).

### Example 178: Preparation of Compound 178

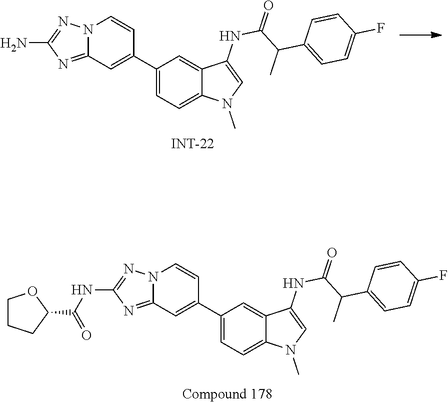 [SMILES: CC(C(=O)Nc1cn(C)c2ccc(-c3ccn4nc(N)nc4c3)cc12)c1ccc(F)cc1 ; "INT-22" ; CC(C(=O)Nc1cn(C)c2ccc(-c3ccn4nc(NC(=O)[C@@H]5CCCO5)nc4c3)cc12)c1ccc(F)cc1]

### Preparation of Compound 178

INT-22 (84 mg, 0.2 mmol) was dissolved in pyridine (1 mL), (S)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 178 with a yield of 71%. MS (ESI, positive ion) m/z: 527.2 [M+H]⁺.

¹H NMR (400 MHz, DMSO-d₆) δ 10.45 (s, 1H), 10.17 (s, 1H), 8.94 (d, J=7.1 Hz, 1H), 8.38 (d, J=1.8 Hz, 1H), 7.94 (d, J=2.0 Hz, 1H), 7.85 (s, 1H), 7.72 (dd, J=8.7, 1.8 Hz, 1H), 7.58-7.48 (m, 4H), 7.21-7.16 (m, 2H), 4.55 (s, 1H), 4.10-3.97 (m, 2H), 3.88-3.83 (m, 1H), 3.80 (s, 3H), 2.33-2.19 (m, 1H), 2.02 (td, J=7.1, 5.5 Hz, 1H), 1.98-1.93 (m, 1H), 1.49 (d, J=7.0 Hz, 3H), 1.44 (s, 1H).

### Example 179: Preparation of Compound 179

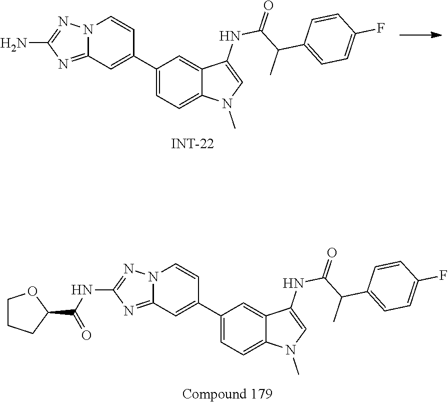 [SMILES: CC(C(=O)Nc1cn(C)c2ccc(-c3ccn4nc(N)nc4c3)cc12)c1ccc(F)cc1 ; "INT-22" ; CC(C(=O)Nc1cn(C)c2ccc(-c3ccn4nc(NC(=O)[C@H]5CCCO5)nc4c3)cc12)c1ccc(F)cc1]

### Preparation of Compound 179

INT-22 (84 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 179 with a yield of 77%. MS (ESI, positive ion) m/z: 527.2 [M+H]⁺.

¹H NMR (400 MHz, DMSO-d₆) δ 10.72 (s, 1H), 10.47 (s, 1H), 8.94 (dd, J=7.1, 0.8 Hz, 1H), 8.22 (s, 1H), 7.96-7.89 (m, 2H), 7.73 (d, J=8.9 Hz, 1H), 7.52 (dd, J=8.4, 5.5 Hz, 2H), 7.43 (dd, J=7.2, 2.0 Hz, 1H), 7.21 (t, J=8.8 Hz, 2H), 4.54 (s, 1H), 4.02 (d, J=8.5 Hz, 6H), 3.85 (q, J=7.0 Hz, 1H), 2.32-2.19 (m, 1H), 2.07-1.84 (m, 3H), 1.50 (d, J=7.0 Hz, 3H).

### Example 180: Preparation of Compound 180

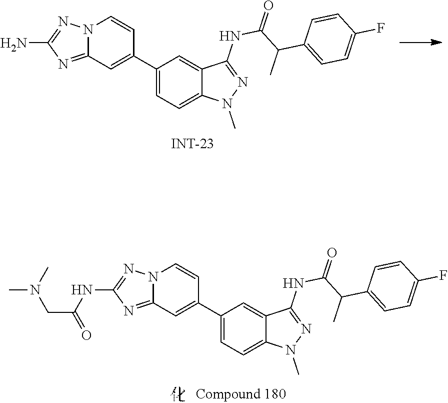 [SMILES: CC(C(=O)Nc1nn(C)c2ccc(-c3ccn4nc(N)nc4c3)cc12)c1ccc(F)cc1 ; "INT-23" ; CC(C(=O)Nc1nn(C)c2ccc(-c3ccn4nc(NC(=O)CN(C)C)nc4c3)cc12)c1ccc(F)cc1]

### Preparation of Compound 180

INT-23 (86 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 180 with a yield of 73%. MS (ESI, positive ion) m/z: 515.2 [M+H]⁺.

¹H NMR (400 MHz, DMSO-d₆) δ 10.76 (s, 2H), 8.94 (d, J=7.1 Hz, 1H), 8.23 (s, 1H), 7.91 (d, J=6.5 Hz, 2H), 7.72 (d, J=8.9 Hz, 1H), 7.52 (t, J=6.9 Hz, 2H), 7.43 (d, J=7.2 Hz, 1H), 7.21 (t, J=8.7 Hz, 2H), 4.06 (s, 1H), 4.00 (s, 3H), 3.39 (s, 2H), 2.42 (s, 6H), 1.50 (d, J=6.8 Hz, 3H).

### Example 181: Preparation of Compound 181

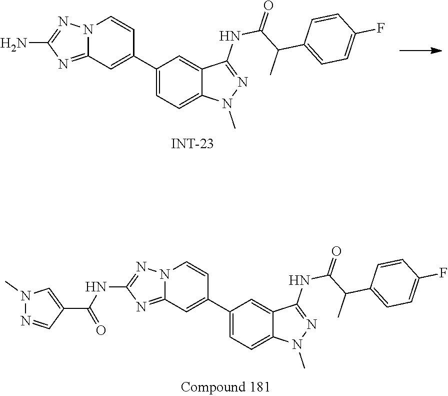 [SMILES: CC(C(=O)Nc1nn(C)c2ccc(-c3ccn4nc(N)nc4c3)cc12)c1ccc(F)cc1 ; "INT-23" ; CC(C(=O)Nc1nn(C)c2ccc(-c3ccn4nc(NC(=O)c5cnn(C)c5)nc4c3)cc12)c1ccc(F)cc1]

### Preparation of Compound 181

INT-23 (86 mg, 0.2 mmol) was dissolved in pyridine (1 mL); 1-methylpyrazole-4-carboxylic acid (38 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 181 with a yield of 62%. MS (ESI, positive ion) m/z: 538.2 [M+H]⁺.

¹H NMR (400 MHz, DMSO-d₆) δ 10.92 (s, 1H), 10.74 (s, 1H), 8.96 (d, J=7.1 Hz, 1H), 8.44 (s, 1H), 8.22 (s, 1H), 8.13 (s, 1H), 7.93 (td, J=4.9, 4.4, 1.8 Hz, 2H), 7.73 (d, J=8.9 Hz, 1H), 7.52 (dd, J=8.5, 5.6 Hz, 2H), 7.42 (dd, J=7.1, 2.0 Hz, 1H), 7.22 (t, J=8.8 Hz, 2H), 4.04 (d, J=7.2 Hz, 2H), 4.01 (s, 3H), 3.92 (s, 3H), 1.50 (d, J=7.0 Hz, 3H).

### Example 182: Preparation of Compound 182

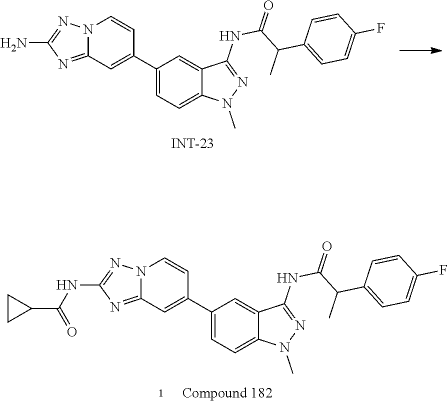 [SMILES: CC(C(=O)Nc1nn(C)c2ccc(-c3ccn4nc(N)nc4c3)cc12)c1ccc(F)cc1 ; "INT-23" ; CC(C(=O)Nc1nn(C)c2ccc(-c3ccn4nc(NC(=O)C5CC5)nc4c3)cc12)c1ccc(F)cc1]

### Preparation of Compound 182

Intermediate INT-23 (86 mg, 0.2 mmol) was dissolved in pyridine (1 mL); cyclopropanecarboxylic acid (28 mg, 0.3 mmol) and 1-propylphosphonic anhydride (111 mg, 0.351 mmol) were added; the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 182 with a yield of 61%. MS (ESI, positive ion) m/z: 498.2 [M+H]⁺.

¹H NMR (400 MHz, DMSO-d₆) δ 11.07 (s, 1H), 10.71 (s, 1H), 8.90 (d, J=7.0 Hz, 1H), 8.21 (s, 1H), 7.90 (d, J=8.9 Hz, 2H), 7.71 (d, J=8.9 Hz, 1H), 7.52 (t, J=6.9 Hz, 2H), 7.39 (d, J=7.2 Hz, 1H), 7.21 (t, J=8.6 Hz, 2H), 4.01 (d, J=11.9 Hz, 4H), 2.05 (d, J=41.0 Hz, 1H), 1.50 (d, J=7.1 Hz, 3H), 0.86 (d, J=6.3 Hz, 4H).

### Example 183: Preparation of Compound 183

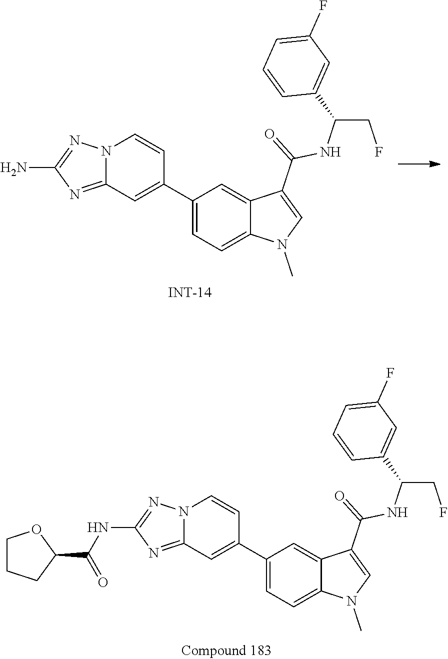 [SMILES: Cn1cc(C(=O)N[C@@H](CF)c2cccc(F)c2)c2cc(-c3ccn4nc(N)nc4c3)ccc21 ; "INT-14" ; Cn1cc(C(=O)N[C@@H](CF)c2cccc(F)c2)c2cc(-c3ccn4nc(NC(=O)[C@H]5CCCO5)nc4c3)ccc21]

### Preparation of Compound 183

INT-14 (86 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 183 with a yield of 67%. MS (ESI, positive ion) m/z: 545.2 [M+H]⁺.

¹H NMR (400 MHz, DMSO-d₆) δ 10.49 (s, 1H), 8.90 (d, J=7.1 Hz, 1H), 8.65 (d, J=8.3 Hz, 1H), 8.55 (d, J=1.8 Hz, 1H), 8.30 (s, 1H), 7.92 (dd, J=2.0, 0.9 Hz, 1H), 7.77 (dd, J=8.7, 1.8 Hz, 1H), 7.70 (d, J=8.6 Hz, 1H), 7.52-7.47 (m, 1H), 7.44 (dd, J=7.9, 6.2 Hz, 1H), 7.36 (d, J=8.4 Hz, 2H), 7.19-7.12 (m, 1H), 5.50 (dt, J=14.8, 7.4 Hz, 1H), 4.78 (d, J=6.4 Hz, 1H), 4.66 (d, J=6.4 Hz, 1H), 4.53 (s, 1H), 4.01 (q, J=7.1, 6.7 Hz, 1H), 3.94 (s, 3H), 3.84 (q, J=7.2 Hz, 1H), 2.30-2.19 (m, 1H), 2.01-1.89 (m, 3H).

### Example 184: Preparation of Compound 184

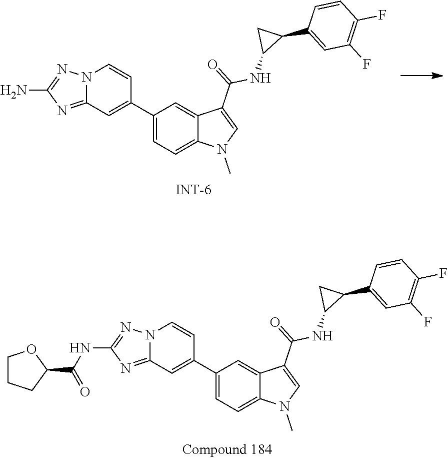 [SMILES: Cn1cc(C(=O)N[C@@H]2C[C@H]2c2ccc(F)c(F)c2)c2cc(-c3ccn4nc(N)nc4c3)ccc21 ; "INT-6" ; Cn1cc(C(=O)N[C@@H]2C[C@H]2c2ccc(F)c(F)c2)c2cc(-c3ccn4nc(NC(=O)[C@H]5CCCO5)nc4c3)ccc21]

### Preparation of Compound 184

INT-6 (91 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 184 with a yield of 71%. MS (ESI, positive ion) m/z: 557.2 [M+H]⁺.

¹H NMR (400 MHz, DMSO-d₆) δ 10.47 (s, 1H), 8.90 (d, J=7.2 Hz, 1H), 8.58 (d, J=1.8 Hz, 1H), 8.31 (d, J=3.8 Hz, 1H), 8.08 (s, 1H), 7.93 (d, J=1.9 Hz, 1H), 7.75 (dd, J=8.6, 1.9 Hz, 1H), 7.68 (d, J=8.7 Hz, 1H), 7.51 (dd, J=7.1, 2.0 Hz, 1H), 7.42-7.23 (m, 2H), 7.09 (dq, J=6.7, 2.8, 2.2 Hz, 1H), 4.54 (s, 1H), 4.02 (dt, J=8.1, 6.7 Hz, 1H), 3.91 (s, 3H), 3.89-3.80 (m, 1H), 3.02 (dq, J=8.0, 3.9 Hz, 1H), 2.25 (dtd, J=11.4, 7.9, 6.1 Hz, 1H), 2.09 (ddd, J=9.5, 6.2, 3.3 Hz, 1H), 2.05-1.81 (m, 3H), 1.39 (dt, J=9.7, 5.3 Hz, 1H), 1.29 (dt, J=7.9, 5.8 Hz, 1H).

### Example 185: Preparation of Compound 185

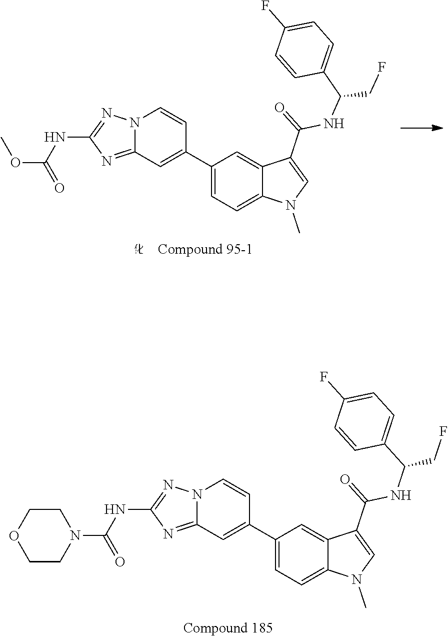 [SMILES: COC(=O)Nc1nc2cc(-c3ccc4c(c3)c(C(=O)N[C@@H](CF)c3ccc(F)cc3)cn4C)ccn2n1 ; "Compound 95-1" ; Cn1cc(C(=O)N[C@@H](CF)c2ccc(F)cc2)c2cc(-c3ccn4nc(NC(=O)N5CCOCC5)nc4c3)ccc21]

### Preparation of Compound 185

Compound 95-1 (100 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 185 with a yield of 66%. MS (ESI, positive ion) m/z: 560.2 [M+H]⁺.

¹H NMR (400 MHz, DMSO-d₆) δ 10.53 (s, 1H), 8.90 (dd, J=7.1, 0.8 Hz, 1H), 8.62 (d, J=8.3 Hz, 1H), 8.55 (d, J=1.8 Hz, 1H), 8.27 (s, 1H), 7.91 (dd, J=1.9, 0.9 Hz, 1H), 7.76 (dd, J=8.7, 1.8 Hz, 1H), 7.69 (d, J=8.6 Hz, 1H), 7.60-7.53 (m, 2H), 7.48 (dd, J=7.2, 2.0 Hz, 1H), 7.28-7.18 (m, 2H), 5.49 (dq, J=15.3, 7.3 Hz, 1H), 4.82-4.70 (m, 1H), 4.70-4.60 (m, 1H), 3.93 (s, 3H), 3.64 (t, J=4.6 Hz, 4H), 3.27 (s, 2H), 2.58-2.55 (m, 4H).

### Example 186: Preparation of Compound 186

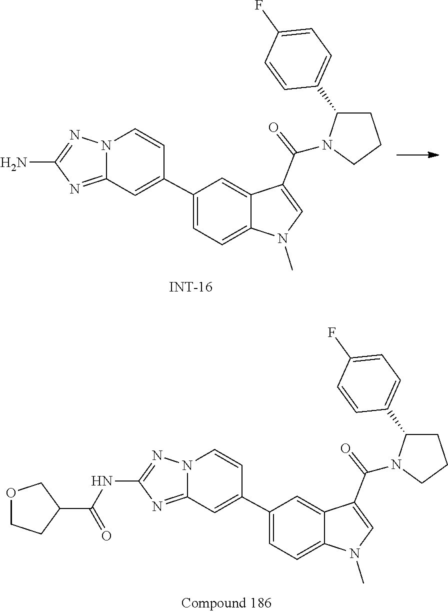 [SMILES: Cn1cc(C(=O)N2CCC[C@H]2c2ccc(F)cc2)c2cc(-c3ccn4nc(N)nc4c3)ccc21 ; "INT-16" ; Cn1cc(C(=O)N2CCC[C@H]2c2ccc(F)cc2)c2cc(-c3ccn4nc(NC(=O)C5CCOC5)nc4c3)ccc21]

### Preparation of Compound 186

INT-16 (90 mg, 0.2 mmol) was dissolved in pyridine (1 mL), tetrahydrofuran-3-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 186 with a yield of 62%. MS (ESI, positive ion) m/z: 553.2 [M+H]⁺.

¹H NMR (400 MHz, DMSO-d6) δ 11.07 (s, 1H), 8.82 (m, 1H), 8.48 (d, J=1.8 Hz, 1H), 8.35 (s, 1H), 7.85 (m, 2H), 7.73 (d, J=8.6 Hz, 1H), 7.59-7.49 (m, 1H), 7.42 (dd, J=7.1, 2.0 Hz, 1H), 7.32 (m, 1H), 7.13 (t, J=8.7 Hz, 2H), 4.71-4.52 (m, 2H), 4.23 (s, 3H), 4.16-3.70 (m, 4H), 2.81 (m, 1H), 2.23-2.04 (m, 2H).

### Example 187: Preparation of Compound 187

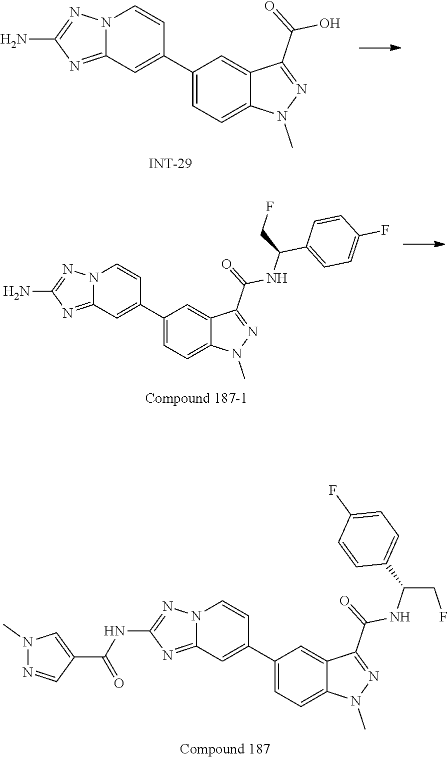 [SMILES: Cn1nc(C(=O)O)c2cc(-c3ccn4nc(N)nc4c3)ccc21 ; "INT-29" ; Cn1nc(C(=O)N[C@@H](CF)c2ccc(F)cc2)c2cc(-c3ccn4nc(N)nc4c3)ccc21 ; Cn1cc(C(=O)Nc2nc3cc(-c4ccc5c(c4)c(C(=O)N[C@@H](CF)c4ccc(F)cc4)nn5C)ccn3n2)cn1]

### Preparation of Compound 187-1

Intermediate INT-29 (321 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-2-fluoro-1-(3-fluorophenyl)ethan-1-amine (188 mg, 1.2 mmol) was added, and the mixture was stirred for reaction at room temperature for 2 h. The reaction mixture was added dropwise to water and stirred for 0.5 h. The mixture was filtered and dried to obtain Compound 187-1 with a yield of 63%. MS (ESI, positive ion) m/z: 448.3 [M+H]⁺.

### Preparation of Compound 187

Compound 187-1 (89 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 187 with a yield of 52%. MS (ESI, positive ion) m/z: 556.2 [M+H]⁺.

¹H NMR (400 MHz, DMSO-d₆) δ 10.94 (s, 1H), 8.94 (dd, J=7.1, 0.8 Hz, 1H), 8.89 (d, J=8.4 Hz, 1H), 8.78 (s, 1H), 8.55 (dd, J=1.8, 0.8 Hz, 1H), 8.43 (s, 1H), 8.13 (d, J=0.8 Hz, 1H), 8.05-7.98 (m, 2H), 7.93 (d, J=9.1 Hz, 1H), 7.78 (s, 1H), 7.52 (ddd, J=8.7, 3.6, 1.9 Hz, 3H), 7.17 (t, J=8.9 Hz, 2H), 5.26 (m, 1H), 4.71-4.52 (m, 2H), 4.23 (s, 3H), 3.86 (s, 3H).

### Example 188: Preparation of Compound 188

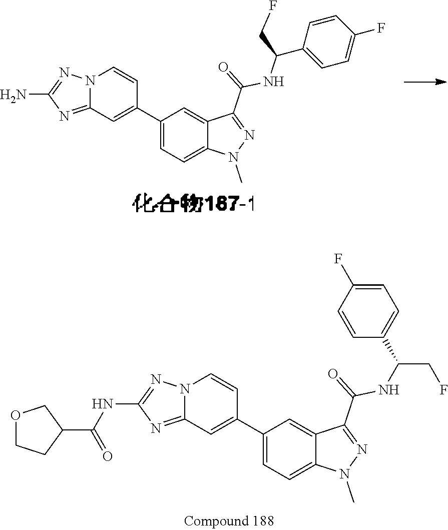

### Preparation of Compound 188

Compound 187-1 (89 mg, 0.2 mmol) was dissolved in pyridine (1 mL), tetrahydrofuran-3-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 188 with a yield of 52%. MS (ESI, positive ion) m/z: 546.2 [M+H]⁺.

¹H NMR (400 MHz, DMSO-d₆) δ 10.94 (s, 1H), 8.94 (dd, J=7.1, 0.8 Hz, 1H), 8.89 (d, J=8.4 Hz, 1H), 8.55 (dd, J=1.8, 0.8 Hz, 1H), 8.43 (s, 1H), 8.13 (d, J=0.8 Hz, 1H), 8.05-7.98 (m, 2H), 7.93 (d, J=9.1 Hz, 1H), 7.52 (ddd, J=8.7, 3.6, 1.9 Hz, 3H), 7.17 (t, J=8.9 Hz, 2H), 5.26 (m, 1H), 4.71-4.52 (m, 2H), 4.23 (s, 3H), 4.16-3.70 (m, 4H), 2.81 (m, 1H), 2.23-2.04 (m, 2H).

### Example 189: Preparation of Compound 189

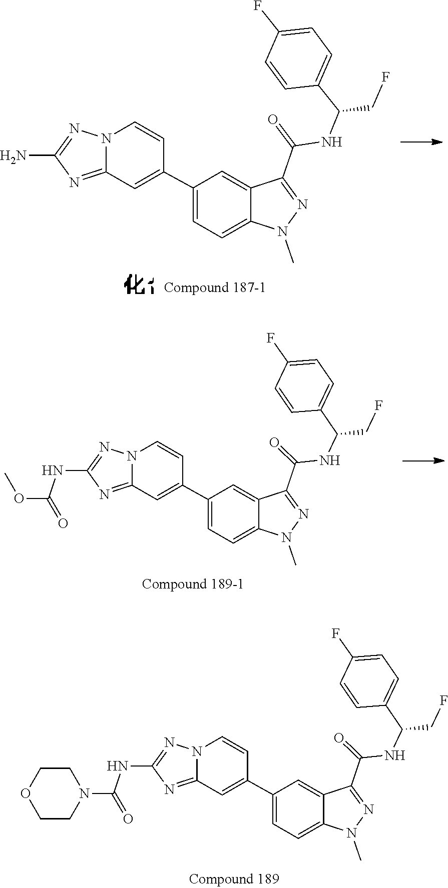 [SMILES: Cn1nc(C(=O)N[C@@H](CF)c2ccc(F)cc2)c2cc(-c3ccn4nc(N)nc4c3)ccc21 ; COC(=O)Nc1nc2cc(-c3ccc4c(c3)c(C(=O)N[C@@H](CF)c3ccc(F)cc3)nn4C)ccn2n1 ; Cn1nc(C(=O)N[C@@H](CF)c2ccc(F)cc2)c2cc(-c3ccn4nc(NC(=O)N5CCOCC5)nc4c3)ccc21]

### Preparation of Compound 189-1

Intermediate 187-1 (447 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 189-1 with a yield of 61%. MS (ESI, positive ion) m/z: 506.2 [M+H]⁺.

### Preparation of Compound 189

Compound 189-1 (101 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 189 with a yield of 58%. MS (ESI, positive ion) m/z: 561.2 [M+H]⁺.

¹H NMR (400 MHz, DMSO-d₆) δ 9.65 (s, 1H), 9.20 (d, J=8.6 Hz, 1H), 8.85 (d, J=7.1 Hz, 1H), 8.52 (s, 1H), 8.03-7.97 (m, 1H), 7.97-7.90 (m, 2H), 7.62-7.54 (m, 2H), 7.49-7.43 (m, 1H), 7.22 (t, J=8.9 Hz, 2H), 5.50 (dq, J=13.5, 8.4, 6.8 Hz, 1H), 5.00-4.73 (m, 1H), 4.69 (ddd, J=46.3, 9.5, 5.6 Hz, 1H), 4.24 (s, 3H), 3.66-3.59 (m, 4H), 3.47 (t, J=4.7 Hz, 4H).

### Example 190: Preparation of Compound 190

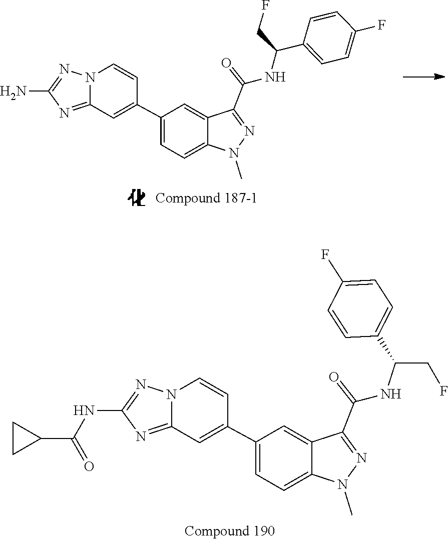 [SMILES: Cn1nc(C(=O)N[C@@H](CF)c2ccc(F)cc2)c2cc(-c3ccn4nc(N)nc4c3)ccc21 ; Cn1nc(C(=O)N[C@@H](CF)c2ccc(F)cc2)c2cc(-c3ccn4nc(NC(=O)C5CC5)nc4c3)ccc21 ; "Compound 190"]

### Preparation of Compound 190

Compound 187-1 (89 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 190 with a yield of 72%. MS (ESI, positive ion) m/z: 516.2 [M+H]⁺.

¹H NMR (400 MHz, DMSO-d₆) δ 10.94 (s, 1H), 8.94 (dd, J=7.1, 0.8 Hz, 1H), 8.89 (d, J=8.4 Hz, 1H), 8.55 (dd, J=1.8, 0.8 Hz, 1H), 8.43 (s, 1H), 8.13 (d, J=0.8 Hz, 1H), 8.05-7.98 (m, 2H), 7.93 (d, J=9.1 Hz, 1H), 7.52 (ddd, J=8.7, 3.6, 1.9 Hz, 3H), 7.17 (t, J=8.9 Hz, 2H), 5.26 (p, J=7.4 Hz, 1H), 4.23 (s, 3H), 4.04-3.93 (m, 2H), 1.55 (d, J=7.1 Hz, 3H).

### Example 191: Preparation of Compound 191

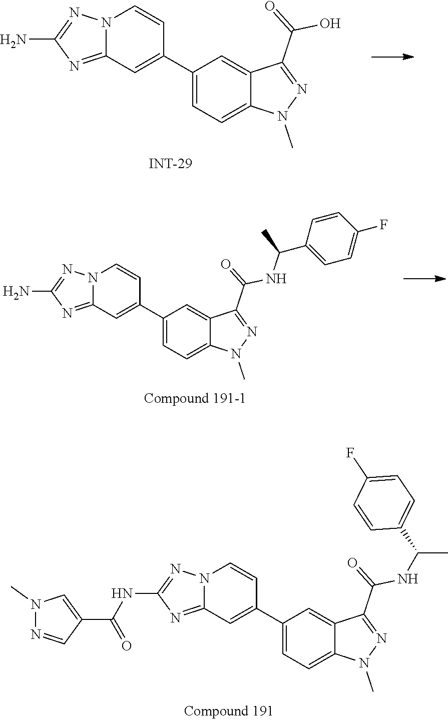

### Preparation of Compound 191-1

Intermediate INT-29 (321 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(3-fluorophenyl)ethan-1-amine (171 mg, 1.2 mmol) was added, and the mixture was stirred for reaction at room temperature for 2 h. The reaction mixture was added dropwise to water and stirred for 0.5 h. The mixture was filtered and dried to obtain Compound 191-1 with a yield of 61%. MS (ESI, positive ion) m/z: 430.3 [M+H]⁺.

### Preparation of Compound 191

Compound 191-1 (86 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 191 with a yield of 52%. MS (ESI, positive ion) m/z: 538.2 [M+H]⁺.

¹H NMR (400 MHz, DMSO-d₆) δ 10.94 (s, 1H), 8.94 (dd, J=7.1, 0.8 Hz, 1H), 8.89 (d, J=8.4 Hz, 1H), 8.55 (dd, J=1.8, 0.8 Hz, 1H), 8.43 (s, 1H), 8.13 (d, J=0.8 Hz, 1H), 8.05-7.98 (m, 2H), 7.93 (d, J=9.1 Hz, 1H), 7.52 (ddd, J=8.7, 3.6, 1.9 Hz, 3H), 7.17 (t, J=8.9 Hz, 2H), 5.26 (p, J=7.4 Hz, 1H), 4.23 (s, 3H), 3.92 (s, 3H), 1.55 (d, J=7.1 Hz, 3H).

### Example 192: Preparation of Compound 192

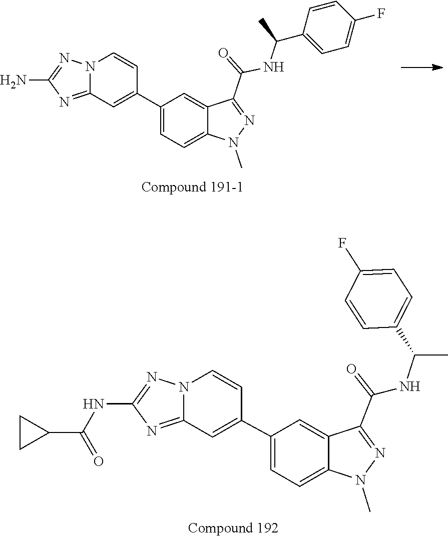 [SMILES: C[C@H](NC(=O)c1nn(C)c2ccc(-c3ccn4nc(N)nc4c3)cc12)c1ccc(F)cc1 ; C[C@H](NC(=O)c1nn(C)c2ccc(-c3ccn4nc(NC(=O)C5CC5)nc4c3)cc12)c1ccc(F)cc1 ; "Compound 192"]

### Preparation of Compound 192

Compound 191-1 (86 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 192 with a yield of 62%. MS (ESI, positive ion) m/z: 498.2 [M+H]⁺.

¹H NMR (400 MHz, DMSO-d₆) δ 11.11 (s, 1H), 8.92-8.86 (m, 2H), 8.56-8.51 (m, 1H), 8.04-7.95 (m, 2H), 7.92 (d, J=8.9 Hz, 1H), 7.56-7.46 (m, 3H), 7.22-7.13 (m, 2H), 5.26 (p, J=7.3 Hz, 1H), 4.22 (s, 3H), 2.45-1.65 (m, −1H), 1.55 (d, J=7.0 Hz, 3H), 0.86 (d, J=6.2 Hz, 4H).

### Example 193: Preparation of Compound 193

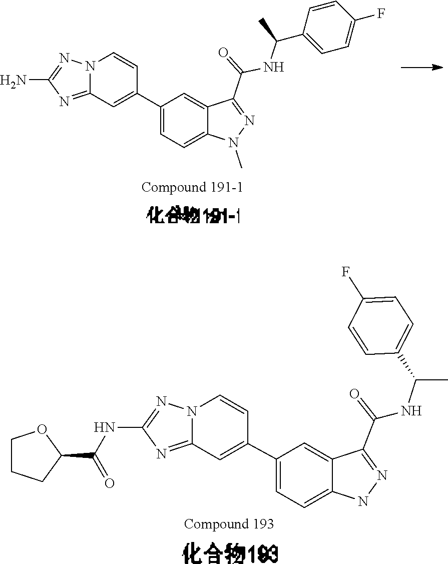 [SMILES: C[C@H](NC(=O)c1nn(C)c2ccc(-c3ccn4nc(N)nc4c3)cc12)c1ccc(F)cc1 ; C[C@H](NC(=O)c1n[nH]c2ccc(-c3ccn4nc(NC(=O)[C@H]5CCCO5)nc4c3)cc12)c1ccc(F)cc1 ; "Compound 193"]

### Preparation of Compound 193

Compound 191-1 (86 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 193 with a yield of 72%. MS (ESI, positive ion) m/z: 528.2 [M+H]⁺.

¹H NMR (400 MHz, DMSO-d₆) δ 10.49 (s, 1H), 8.92 (dd, J=7.1, 0.8 Hz, 1H), 8.87 (d, J=8.4 Hz, 1H), 8.56-8.51 (m, 1H), 8.00 (ddd, J=5.5, 3.9, 1.8 Hz, 2H), 7.92 (d, J=8.9 Hz, 1H), 7.54-7.50 (m, 3H), 7.22-7.12 (m, 2H), 5.26 (p, J=7.1 Hz, 1H), 4.54 (s, 1H), 4.22 (s, 3H), 4.02 (dt, J=8.0, 6.7 Hz, 1H), 3.90-3.80 (m, 1H), 2.25 (dtd, J=11.4, 8.0, 6.1 Hz, 1H), 2.03-1.88 (m, 3H), 1.56 (d, J=7.1 Hz, 3H).

### Example 194: Preparation of Compound 194

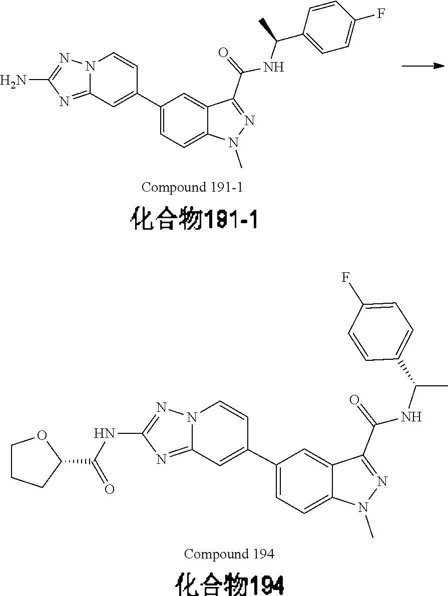 [SMILES: C[C@H](NC(=O)c1nn(C)c2ccc(-c3ccn4nc(N)nc4c3)cc12)c1ccc(F)cc1 ; C[C@H](NC(=O)c1nn(C)c2ccc(-c3ccn4nc(NC(=O)[C@@H]5CCCO5)nc4c3)cc12)c1ccc(F)cc1 ; "Compound 194"]

### Preparation of Compound 194

Compound 191-1 (86 mg, 0.2 mmol) was dissolved in pyridine (1 mL), (S)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 194 with a yield of 66%. MS (ESI, positive ion) m/z: 528.2 [M+H]⁺.

¹H NMR (400 MHz, DMSO-d₆) δ 10.50 (s, 1H), 8.95-8.85 (m, 2H), 8.56-8.50 (m, 1H), 8.03-7.96 (m, 2H), 7.92 (d, J=9.0 Hz, 1H), 7.52 (td, J=5.6, 2.6 Hz, 3H), 7.22-7.12 (m, 2H), 5.26 (p, J=7.2 Hz, 1H), 4.53 (s, 1H), 4.22 (s, 3H), 4.01 (q, J=6.9 Hz, 1H), 3.84 (td, J=7.6, 6.5 Hz, 1H), 2.30-1.92 (m, 2H), 1.97-1.82 (m, 2H), 1.55 (d, J=7.1 Hz, 3H).

### Example 195: Preparation of Compound 195

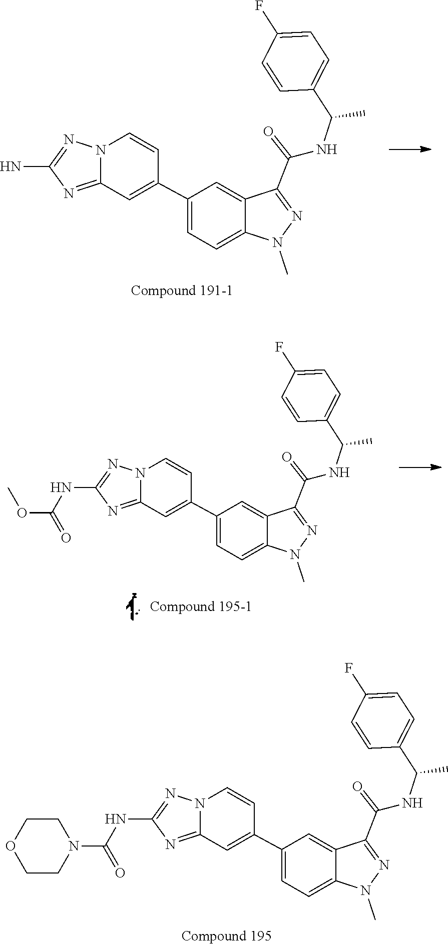

### Preparation of Compound 195-1

Intermediate 191-1 (429 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 195-1 with a yield of 61%. MS (ESI, positive ion) m/z: 488.2 [M+H]⁺.

### Preparation of Compound 195

Compound 195-1 (97 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 195 with a yield of 58%. MS (ESI, positive ion) m/z: 543.2 [M+H]⁺.

¹H NMR (400 MHz, DMSO-d₆) δ 9.63 (s, 1H), 8.86 (t, J=7.3 Hz, 2H), 8.52 (d, J=1.8 Hz, 1H), 7.99 (dd, J=8.9, 1.8 Hz, 1H), 7.95-7.85 (m, 2H), 7.57-7.49 (m, 2H), 7.46 (dd, J=7.2, 2.0 Hz, 1H), 7.21-7.12 (m, 2H), 5.26 (p, J=7.3 Hz, 1H), 4.22 (s, 3H), 3.63 (t, J=4.7 Hz, 4H), 3.48 (t, J=4.8 Hz, 4H), 1.55 (d, J=7.0 Hz, 3H).

### Example 196: Preparation of Compound 196

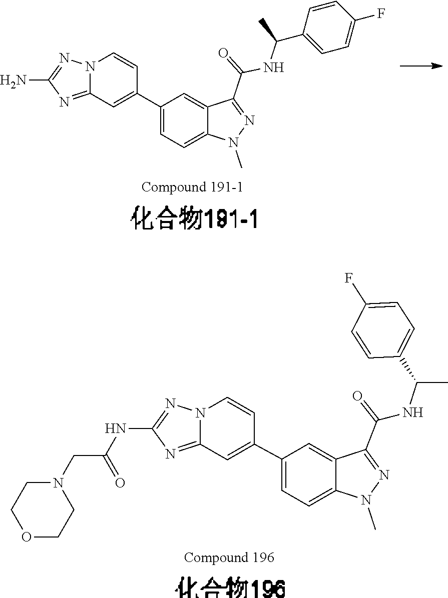 [SMILES: C[C@H](NC(=O)c1nn(C)c2ccc(-c3ccn4nc(N)nc4c3)cc12)c1ccc(F)cc1 ; C[C@H](NC(=O)c1nn(C)c2ccc(-c3ccn4nc(NC(=O)CN5CCOCC5)nc4c3)cc12)c1ccc(F)cc1 ; "Compound 196"]

### Preparation of Compound 196

Compound 191-1 (86 mg, 0.2 mmol) was dissolved in pyridine (1 mL), morpholineacetic acid (43 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 196 with a yield of 71%. MS (ESI, positive ion) m/z: 557.2 [M+H]⁺.

¹H NMR (400 MHz, DMSO-d₆) δ 10.53 (s, 1H), 8.85 (dd, J=17.4, 7.7 Hz, 2H), 8.49 (d, J=1.7 Hz, 1H), 7.93 (dd, J=6.5, 1.9 Hz, 2H), 7.86 (d, J=8.9 Hz, 1H), 7.53-7.45 (m, 3H), 7.14 (t, J=8.9 Hz, 2H), 5.24 (t, J=7.5 Hz, 1H), 4.18 (s, 3H), 3.62 (t, J=4.5 Hz, 4H), 3.26 (s, 2H), 2.54 (t, J=4.6 Hz, 4H), 1.53 (d, J=7.0 Hz, 3H).

### Example 197: Preparation of Compound 197

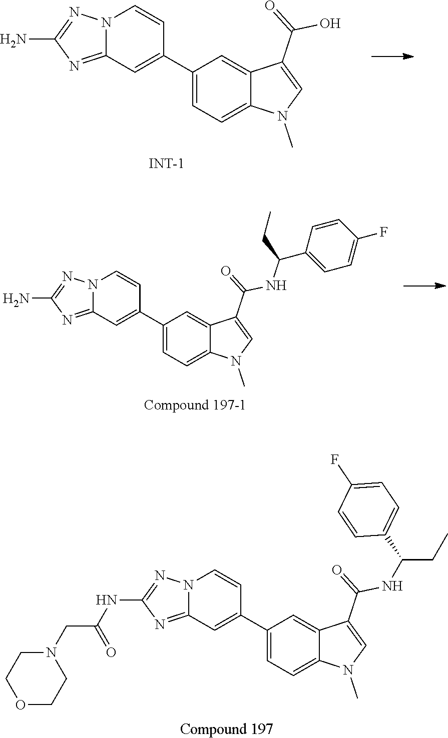

### Preparation of Compound 197-1

Intermediate INT-1 (306 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(3-fluorophenyl)propan-1-amine (188 mg, 1.2 mmol) was added, and the mixture was stirred for reaction at room temperature for 2 h. The reaction mixture was added dropwise to water and stirred for 0.5 h. The mixture was filtered and dried to obtain Compound 197-1 with a yield of 64%. MS (ESI, positive ion) m/z: 443.3 [M+H]⁺.

### Preparation of Compound 197

Compound 197-1 (88 mg, 0.2 mmol) was dissolved in pyridine (1 mL), morpholineacetic acid (43 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 197 with a yield of 61%. MS (ESI, positive ion) m/z: 570.2 [M+H]⁺.

¹H NMR (400 MHz, DMSO-d₆) δ 10.54 (s, 1H), 8.89 (d, J=7.1 Hz, 1H), 8.55 (d, J=1.9 Hz, 1H), 8.38 (d, J=8.1 Hz, 1H), 8.23 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 5.22 (p, J=7.2 Hz, 1H), 3.91 (s, 3H), 3.64 (t, J=4.6 Hz, 4H), 3.27 (s, 2H), 2.56 (dd, J=5.4, 3.4 Hz, 4H), 1.50 (d, J=7.0 Hz, 3H).

### Example 198: Preparation of Compound 198

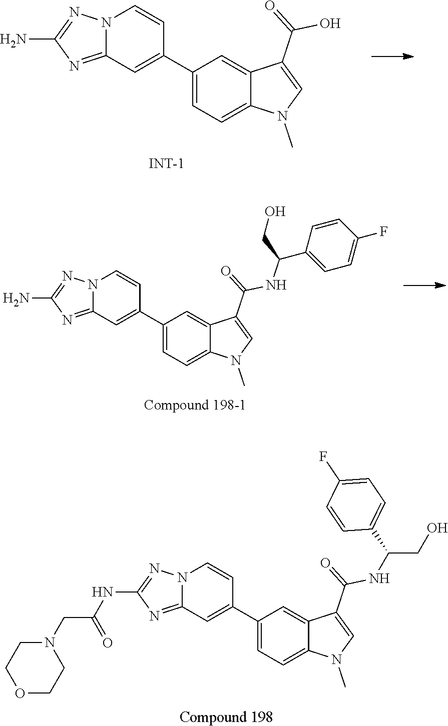

### Preparation of Compound 198-1

Intermediate INT-1 (306 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-1-(3-fluorophenyl)ethanol-1-amine (190 mg, 1.2 mmol) was added, and the mixture was stirred for reaction at room temperature for 2 h. The reaction mixture was added dropwise to water and stirred for 0.5 h. The mixture was filtered and dried to obtain Compound 198-1 with a yield of 54%. MS (ESI, positive ion) m/z: 443.3 [M+H]⁺.

### Preparation of Compound 198

Compound 198-1 (88 mg, 0.2 mmol) was dissolved in pyridine (1 mL), morpholineacetic acid (43 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 198 with a yield of 61%. MS (ESI, positive ion) m/z: 571.2 [M+H]⁺.

¹H NMR (400 MHz, DMSO-d₆) δ 10.54 (s, 1H), 8.89 (d, J=7.1 Hz, 1H), 8.55 (d, J=1.9 Hz, 1H), 8.38 (d, J=8.1 Hz, 1H), 8.23 (s, 1H), 7.90 (d, J=1.9 Hz, 1H), 7.74 (dd, J=8.6, 1.8 Hz, 1H), 7.67 (d, J=8.7 Hz, 1H), 7.47 (ddt, J=5.8, 4.1, 1.7 Hz, 3H), 7.22-7.12 (m, 2H), 5.22 (p, J=7.2 Hz, 1H), 3.95 (s, 1H), 3.81-3.72 (m, 2H), 3.64 (t, J=4.6 Hz, 4H), 3.27 (s, 2H), 2.56 (dd, J=5.4, 3.4 Hz, 4H), 1.50 (d, J=7.0 Hz, 3H).

### Example 199: Preparation of Compound 199

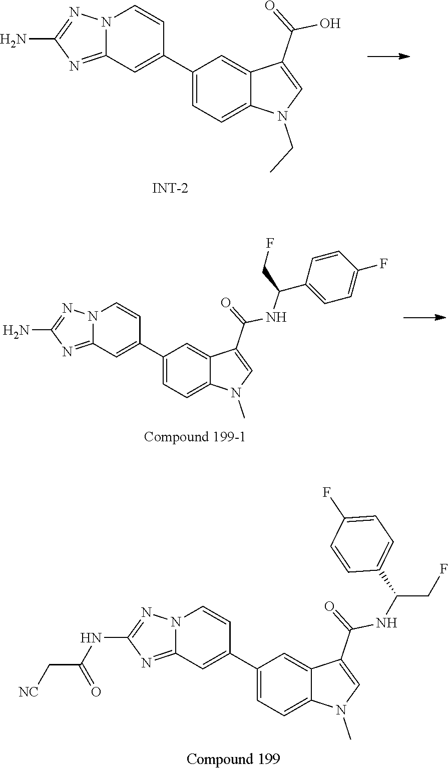

### Preparation of Compound 199-1

Intermediate INT-2 (320 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (R)-2-fluoro-1-(3-fluorophenyl)ethan-1-amine (188 mg, 1.2 mmol) was added, and the mixture was stirred for reaction at room temperature for 2 h. The reaction mixture was added dropwise to water and stirred for 0.5 h. The mixture was filtered and dried to obtain Compound 199-1 with a yield of 67%. MS (ESI, positive ion) m/z: 461.3 [M+H]⁺.

### Preparation of Compound 199

Compound 199-1 (92 mg, 0.2 mmol) was dissolved in pyridine (1 mL), cyanoacetic acid (25 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 199 with a yield of 57%. MS (ESI, positive ion) m/z: 527.2 [M+H]⁺.

¹H NMR (400 MHz, DMSO-d₆) δ 11.29 (s, 1H), 8.92 (d, J=7.1 Hz, 1H), 8.62 (d, J=8.2 Hz, 1H), 8.55 (d, J=1.8 Hz, 1H), 8.27 (s, 1H), 7.94 (d, J=1.8 Hz, 1H), 7.77 (dd, J=8.6, 1.9 Hz, 1H), 7.69 (d, J=8.7 Hz, 1H), 7.57-7.49 (m, 3H), 7.23 (t, J=8.8 Hz, 2H), 5.53-5.43 (m, 1H), 4.76 (d, J=8.0 Hz, 1H), 4.67-4.61 (m, 1H), 4.04-3.93 (m, 2H), 2.72-2.55 (m, 1H), 2.35 (s, 1H), 1.50 (d, J=7.2 Hz, 3H).

### Example 200: Preparation of Compound 200

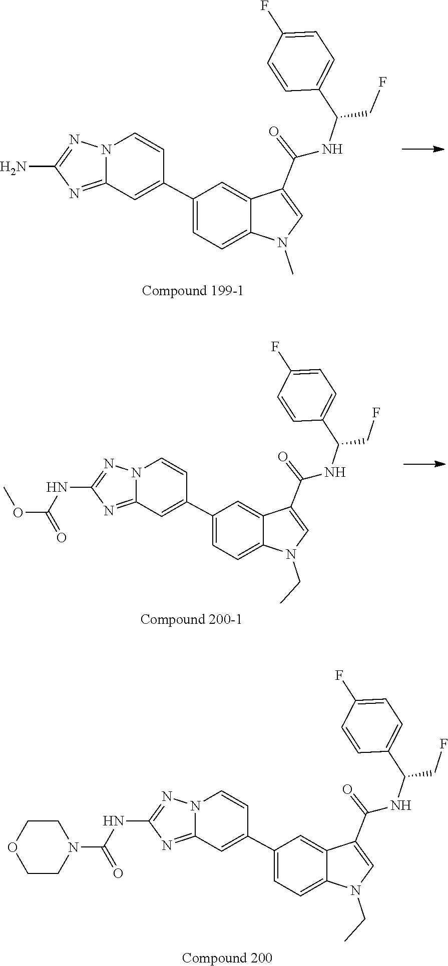 [SMILES: Cn1cc(C(=O)N[C@@H](CF)c2ccc(F)cc2)c2cc(-c3ccn4nc(N)nc4c3)ccc21 ; "Compound 199-1" ; CCn1cc(C(=O)N[C@@H](CF)c2ccc(F)cc2)c2cc(-c3ccn4nc(NC(=O)OC)nc4c3)ccc21 ; "Compound 200-1" ; CCn1cc(C(=O)N[C@@H](CF)c2ccc(F)cc2)c2cc(-c3ccn4nc(NC(=O)N5CCOCC5)nc4c3)ccc21 ; "Compound 200"]

### Preparation of Compound 200-1

Intermediate 199-1 (460 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 200-1 with a yield of 55%. MS (ESI, positive ion) m/z: 519.2 [M+H]⁺.

### Preparation of Compound 200

Compound 200-1 (104 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 200 with a yield of 58%. MS (ESI, positive ion) m/z: 574.2 [M+H]⁺.

¹H NMR (400 MHz, DMSO-d₆) δ 11.29 (s, 1H), 8.92 (d, J=7.1 Hz, 1H), 8.62 (d, J=8.2 Hz, 1H), 8.55 (d, J=1.8 Hz, 1H), 8.27 (s, 1H), 7.94 (d, J=1.8 Hz, 1H), 7.77 (dd, J=8.6, 1.9 Hz, 1H), 7.69 (d, J=8.7 Hz, 1H), 7.57-7.49 (m, 3H), 7.23 (t, J=8.8 Hz, 2H), 5.53-5.43 (m, 1H), 4.76 (d, J=8.0 Hz, 1H), 4.67-4.61 (m, 1H), 3.66-3.58 (m, 4H), 3.48 (t, J=4.8 Hz, 4H), 2.72-2.55 (m, 1H), 2.35 (s, 1H), 1.50 (d, J=7.2 Hz, 3H).

### Example 201: Preparation of Compound 201

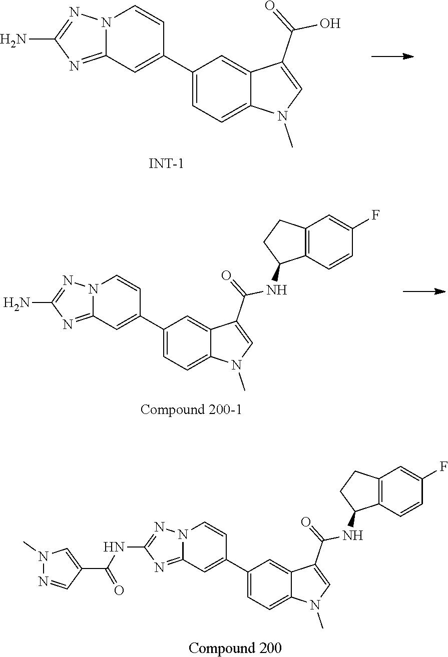

### Preparation of Compound 201-1

Intermediate INT-1 (306 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-5-fluoro-2,3-dihydro-1H-inden-1-amine (181 mg, 1.2 mmol) was added, and the mixture was stirred for reaction at room temperature for 2 h; the reaction mixture was added dropwise into water and stirred for 0.5 h; the mixture was filtered and dried to obtain Compound 201-1 with a yield of 58%. MS (ESI, positive ion) m/z: 441.3 [M+H]⁺.

### Preparation of Compound 201

Compound 201-1 (88 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 201 with a yield of 53%. MS (ESI, positive ion) m/z: 549.2 [M+H]⁺.

¹H NMR (400 MHz, DMSO-d₆) δ 10.92 (s, 1H), 8.94 (dd, J=7.1, 0.8 Hz, 1H), 8.66 (d, J=1.7 Hz, 1H), 8.43 (s, 1H), 8.34 (d, J=8.4 Hz, 1H), 8.18-8.11 (m, 2H), 7.95 (dd, J=2.0, 0.9 Hz, 1H), 7.78 (dd, J=8.7, 1.9 Hz, 1H), 7.69 (d, J=8.6 Hz, 1H), 7.53 (dd, J=7.2, 2.0 Hz, 1H), 7.31 (dd, J=8.3, 5.4 Hz, 1H), 7.16-7.10 (m, 1H), 7.07-6.99 (m, 1H), 5.58 (q, J=8.2 Hz, 1H), 3.92 (s, 3H), 3.90 (s, 3H), 3.04 (t, J=11.8 Hz, 1H), 2.89 (dt, J=16.5, 8.4 Hz, 1H), 2.58-2.54 (m, 1H), 2.12-1.98 (m, 1H).

### Example 202: Preparation of Compound 202

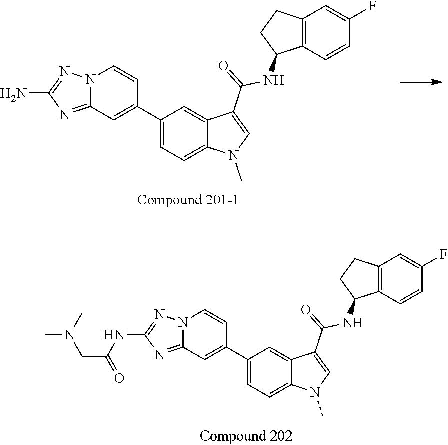

### Preparation of Compound 202

Compound 201-1 (88 mg, 0.2 mmol) was dissolved in pyridine (1 mL), 2-(dimethylamino)acetic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 202 with a yield of 58%. MS (ESI, positive ion) m/z: 526.2 [M+H]⁺.

¹H NMR (400 MHz, DMSO-d₆) δ 10.45 (s, 1H), 8.91 (d, J=7.3 Hz, 1H), 8.64 (d, J=1.9 Hz, 1H), 8.33 (d, J=8.4 Hz, 1H), 8.16 (s, 1H), 7.93 (d, J=2.0 Hz, 1H), 7.76 (dd, J=8.6, 1.9 Hz, 1H), 7.68 (d, J=8.6 Hz, 1H), 7.51 (dd, J=7.1, 2.0 Hz, 1H), 7.31 (dd, J=8.4, 5.4 Hz, 1H), 7.13 (d, J=9.9 Hz, 1H), 7.03 (t, J=8.9 Hz, 1H), 5.58 (q, J=7.9 Hz, 1H), 3.89 (s, 4H), 3.21 (s, 2H), 3.09-2.99 (m, 1H), 2.89 (dt, J=16.4, 8.4 Hz, 1H), 2.33 (s, 5H), 2.04 (dq, J=12.5, 8.7 Hz, 1H).

### Example 203: Preparation of Compound 203

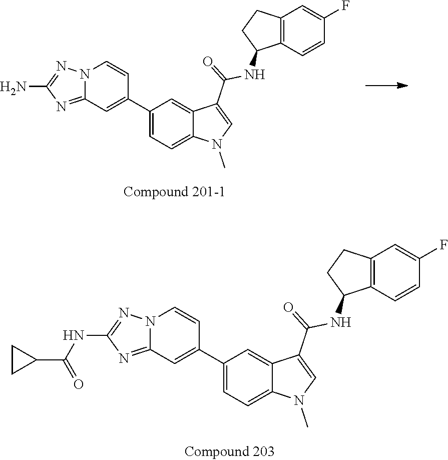 [SMILES: Cn1cc(C(=O)N[C@H]2CCc3cc(F)ccc32)c2cc(-c3ccn4nc(N)nc4c3)ccc21 ; "Compound 201-1" ; Cn1cc(C(=O)N[C@H]2CCc3cc(F)ccc32)c2cc(-c3ccn4nc(NC(=O)C5CC5)nc4c3)ccc21]

### Preparation of Compound 203

Compound 201-1 (88 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 203 with a yield of 68%. MS (ESI, positive ion) m/z: 509.2 [M+H]⁺.

¹H NMR (400 MHz, DMSO-d₆) δ 11.09 (s, 1H), 8.89 (d, J=7.0 Hz, 1H), 8.65 (d, J=1.8 Hz, 1H), 8.34 (d, J=8.3 Hz, 1H), 8.16 (s, 1H), 7.93 (d, J=2.0 Hz, 1H), 7.77 (dd, J=8.7, 2.0 Hz, 1H), 7.69 (d, J=8.7 Hz, 1H), 7.50 (dd, J=7.1, 2.0 Hz, 1H), 7.32 (dd, J=8.4, 5.3 Hz, 1H), 7.14 (d, J=10.0 Hz, 1H), 7.08-6.99 (m, 1H), 5.58 (q, J=8.0 Hz, 1H), 3.90 (s, 3H), 3.05 (dd, J=17.5, 8.1 Hz, 1H), 2.90 (dt, J=16.5, 8.5 Hz, 1H), 2.60-2.55 (m, 1H), 2.23-1.93 (m, 2H), 0.87 (d, J=6.1 Hz, 4H).

### Example 204: Preparation of Compound 204

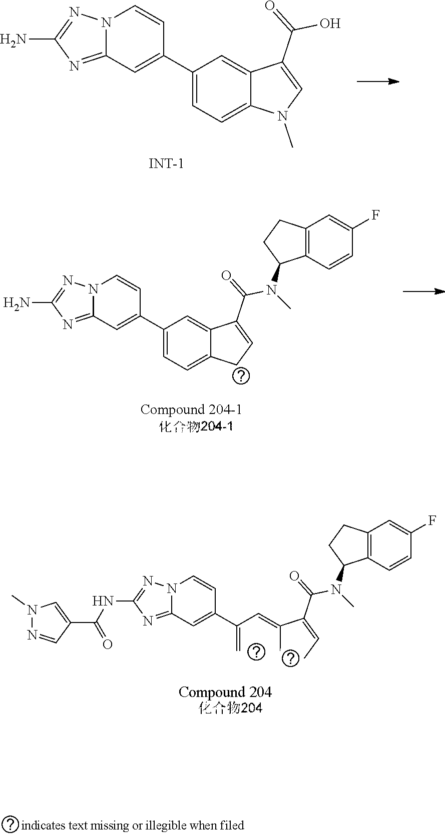

### Preparation of Compound 204-1

Intermediate INT-1 (306 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-5-fluoro-2,3-dihydro-1H-inden-1-amine-1-methyl (197 mg, 1.2 mmol) was added, and the mixture was stirred for reaction at room temperature for 2 h; the reaction mixture was added dropwise into water and stirred for 0.5 h; the mixture was filtered and dried to obtain Compound 204-1 with a yield of 49%. MS (ESI, positive ion) m/z: 454.3 [M+H]⁺.

### Preparation of Compound 204

Compound 204-1 (91 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 204 with a yield of 51%. MS (ESI, positive ion) m/z: 563.2 [M+H]⁺.

¹H NMR (400 MHz, DMSO-d₆) δ 10.92 (s, 1H), 8.93 (d, J=7.1 Hz, 1H), 8.66 (d, J=1.8 Hz, 1H), 8.44 (s, 1H), 8.39 (d, J=8.3 Hz, 1H), 8.18 (s, 1H), 8.14 (s, 1H), 7.95 (d, J=1.9 Hz, 1H), 7.77 (dd, J=8.6, 1.9 Hz, 1H), 7.69 (d, J=8.6 Hz, 1H), 7.53 (dd, J=7.2, 2.0 Hz, 1H), 7.31 (dd, J=8.8, 5.2 Hz, 1H), 7.06 (t, J=7.8 Hz, 2H), 5.62 (t, J=8.1 Hz, 1H), 3.91 (d, J=7.9 Hz, 6H), 3.05-2.94 (m, 1H), 3.26 (s, 3H), 2.85 (dt, J=16.4, 8.7 Hz, 1H), 2.57 (d, J=8.8 Hz, 1H), 2.05 (dq, J=12.3, 8.8 Hz, 1H).

### Example 205: Preparation of Compound 205

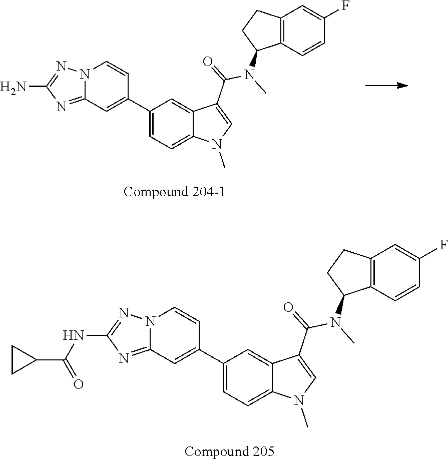 [SMILES: CN(C(=O)c1cn(C)c2ccc(-c3ccn4nc(N)nc4c3)cc12)[C@H]1CCc2cc(F)ccc21 ; "Compound 204-1" ; CN(C(=O)c1cn(C)c2ccc(-c3ccn4nc(NC(=O)C5CC5)nc4c3)cc12)[C@H]1CCc2cc(F)ccc21]

### Preparation of Compound 205

Compound 204-1 (91 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 205 with a yield of 67%. MS (ESI, positive ion) m/z: 523.2 [M+H]⁺.

¹H NMR (400 MHz, DMSO-d₆) δ 11.09 (s, 1H), 8.89 (d, J=7.0 Hz, 1H), 8.65 (d, J=1.8 Hz, 1H), 8.34 (d, J=8.3 Hz, 1H), 8.16 (s, 1H), 7.93 (d, J=2.0 Hz, 1H), 7.77 (dd, J=8.7, 2.0 Hz, 1H), 7.50 (dd, J=7.1, 2.0 Hz, 1H), 7.32 (dd, J=8.4, 5.3 Hz, 1H), 7.14 (d, J=10.0 Hz, 1H), 7.08-6.99 (m, 1H), 5.58 (q, J=8.0 Hz, 1H), 3.90 (s, 3H), 3.26 (s, 3H), 3.05 (dd, J=17.5, 8.1 Hz, 1H), 2.90 (dt, J=16.5, 8.5 Hz, 1H), 2.60-2.55 (m, 1H), 2.23-1.93 (m, 2H), 0.87 (d, J=6.1 Hz, 4H).

### Example 206: Preparation of Compound 206

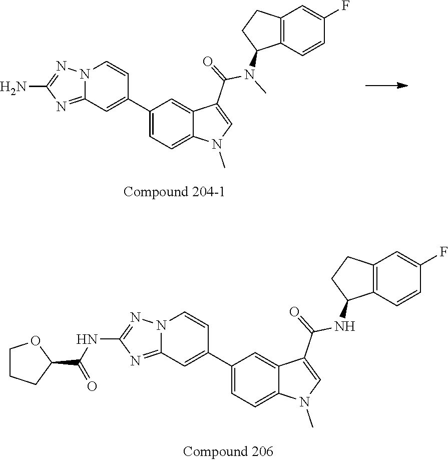 [SMILES: CN(C(=O)c1cn(C)c2ccc(-c3ccn4nc(N)nc4c3)cc12)[C@H]1CCc2cc(F)ccc21 ; "Compound 204-1" ; Cn1cc(C(=O)N[C@H]2CCc3cc(F)ccc32)c2cc(-c3ccn4nc(NC(=O)[C@H]5CCCO5)nc4c3)ccc21]

### Preparation of Compound 206

Compound 204-1 (91 mg, 0.2 mmol) was dissolved in pyridine (1 mL), (R)-tetrahydrofuran-2-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 206 with a yield of 67%. MS (ESI, positive ion) m/z: 553.2 [M+H]⁺.

¹H NMR (400 MHz, DMSO-d₆) δ 11.09 (s, 1H), 8.89 (d, J=7.0 Hz, 1H), 8.65 (d, J=1.8 Hz, 1H), 8.34 (d, J=8.3 Hz, 1H), 8.16 (s, 1H), 7.93 (d, J=2.0 Hz, 1H), 7.77 (dd, J=8.7, 2.0 Hz, 1H), 7.50 (dd, J=7.1, 2.0 Hz, 1H), 7.32 (dd, J=8.4, 5.3 Hz, 1H), 7.14 (d, J=10.0 Hz, 1H), 7.08-6.99 (m, 1H), 5.58 (q, J=7.9 Hz, 1H), 4.54 (s, 1H), 4.02 (q, J=7.0 Hz, 1H), 3.89 (s, 3H), 3.31 (s, 3H), 3.85 (q, J=7.2 Hz, 1H), 3.09-2.99 (m, 1H), 2.89 (dt, J=16.3, 8.5 Hz, 1H), 2.59-2.54 (m, 1H), 2.31-2.16 (m, 1H), 2.11-1.83 (m, 4H).

### Example 207: Preparation of Compound 207

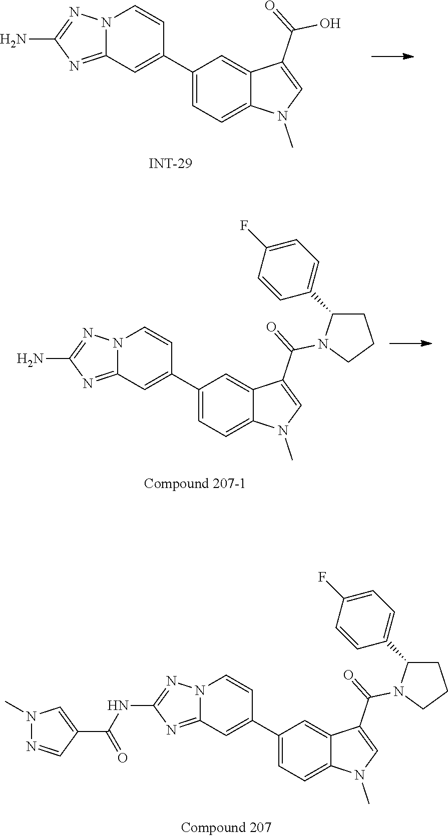

### Preparation of Compound 207-1

Intermediate INT-29 (307 mg, 1.0 mmol) was dissolved in dimethyl sulfoxide (10 mL); N,N-diisopropylethylamine (258 mg, 2 mmol) and HATU (570 mg, 1.5 mmol) were added, and the mixture was stirred at room temperature for 1 h. (S)-2-(4-fluorophenyl)pyrrolidine (198 mg, 1.2 mmol) was added, and the mixture was stirred for reaction at room temperature for 2 h; the reaction mixture was added dropwise into water and stirred for 0.5 h; the mixture was filtered and dried to obtain Compound 207-1 with a yield of 49%. MS (ESI, positive ion) m/z: 456.3 [M+H]⁺.

### Preparation of Compound 207

Compound 207-1 (91 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 207 with a yield of 51%. MS (ESI, positive ion) m/z: 564.2 [M+H]⁺.

¹H NMR (400 MHz, DMSO-d6) δ 11.07 (s, 1H), 8.82 (m, 2H), 8.48 (d, J=1.8 Hz, 1H), 7.85 (m, 2H), 7.73 (d, J=8.6 Hz, 1H), 7.59-7.49 (m, 1H), 7.42 (dd, J=7.1, 2.0 Hz, 1H), 7.32 (dd, J=8.5, 5.5 Hz, 2H), 7.13 (t, J=8.7 Hz, 2H), 5.05 (m, 1H), 3.95 (s, 3H), 3.71 (s, 3H), 1.89 (d, J=6.8 Hz, 3H).

### Example 208: Preparation of Compound 208

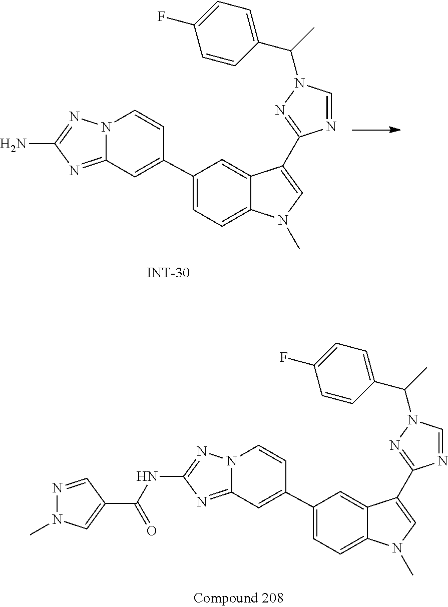 [SMILES: CC(c1ccc(F)cc1)n1cnc(-c2cn(C)c3ccc(-c4ccn5nc(N)nc5c4)cc23)n1 ; "INT-30" ; CC(c1ccc(F)cc1)n1cnc(-c2cn(C)c3ccc(-c4ccn5nc(NC(=O)c6cnn(C)c6)nc5c4)cc23)n1]

### Preparation of Compound 208

Compound INT-30 (90 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 208 with a yield of 53%. MS (ESI, positive ion) m/z: 561.2 [M+H]⁺.

¹H NMR (400 MHz, DMSO-d₆) δ 11.08 (s, 1H), 8.86 (d, J=7.0 Hz, 1H), 8.50 (m, 2H), 8.19 (s, 1H), 7.95 (s, 1H), 7.89 (d, J=1.9 Hz, 1H), 7.82 (m, 2H), 7.73 (d, J=8.6 Hz, 1H), 7.42 (m, 2H), 7.36 (dd, J=8.7, 5.5 Hz, 2H), 7.18 (t, J=8.9 Hz, 2H), 5.05 (m, 1H), 3.95 (s, 3H), 3.71 (s, 3H), 1.89 (d, J=6.8 Hz, 3H).

### Example 209: Preparation of Compound 209

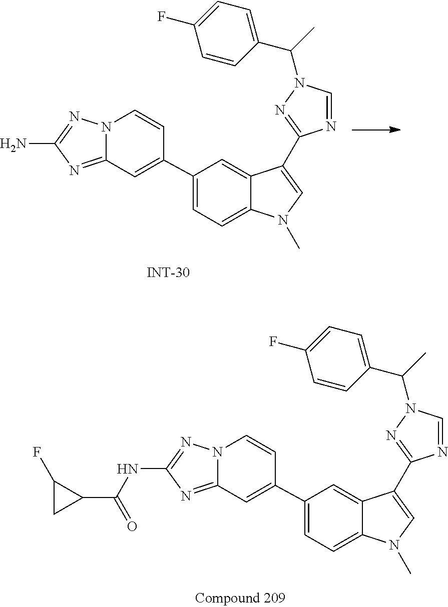 [SMILES: CC(c1ccc(F)cc1)n1cnc(-c2cn(C)c3ccc(-c4ccn5nc(N)nc5c4)cc23)n1 ; "INT-30" ; CC(c1ccc(F)cc1)n1cnc(-c2cn(C)c3ccc(-c4ccn5nc(NC(=O)C6CC6F)nc5c4)cc23)n1]

### Preparation of Compound 209

Compound INT-30 (90 mg, 0.2 mmol) was dissolved in pyridine (1 mL), 2-fluorocyclopropanecarboxylic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 209 with a yield of 56%. MS (ESI, positive ion) m/z: 539.2 [M+H]⁺.

¹H NMR (400 MHz, DMSO-d₆) δ 11.08 (s, 1H), 8.86 (d, J=7.0 Hz, 1H), 8.50 (d, J=1.8 Hz, 1H), 8.19 (s, 1H), 7.95 (s, 1H), 7.89 (d, J=1.9 Hz, 1H), 7.82 (m, 2H), 7.73 (d, J=8.6 Hz, 1H), 7.42 (dd, J=7.2, 2.0 Hz, 1H), 7.36 (dd, J=8.7, 5.5 Hz, 2H), 7.18 (t, J=8.9 Hz, 2H), 6.10 (d, J=6.8 Hz, 1H), 4.71-4.59 (m, 1H), 3.95 (s, 3H), 1.89 (d, J=6.8 Hz, 3H), 0.92-0.78 (m, 3H).

### Example 210: Preparation of Compound 210

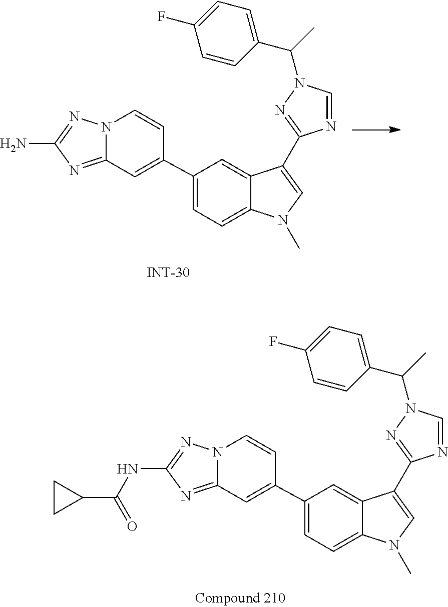 [SMILES: CC(c1ccc(F)cc1)n1cnc(-c2cn(C)c3ccc(-c4ccn5nc(N)nc5c4)cc23)n1 ; "INT-30" ; CC(c1ccc(F)cc1)n1cnc(-c2cn(C)c3ccc(-c4ccn5nc(NC(=O)C6CC6)nc5c4)cc23)n1]

### Preparation of Compound 210

Compound INT-30 (90 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 210 with a yield of 69%. MS (ESI, positive ion) m/z: 521.2 [M+H]⁺.

¹H NMR (400 MHz, DMSO-d₆) δ 11.08 (s, 1H), 8.86 (d, J=7.0 Hz, 1H), 8.50 (d, J=1.8 Hz, 1H), 8.19 (s, 1H), 7.95 (s, 1H), 7.89 (d, J=1.9 Hz, 1H), 7.82 (m, 2H), 7.73 (d, J=8.6 Hz, 1H), 7.42 (dd, J=7.2, 2.0 Hz, 1H), 7.36 (dd, J=8.7, 5.5 Hz, 2H), 7.18 (t, J=8.9 Hz, 2H), 6.10 (d, J=6.8 Hz, 1H), 3.95 (s, 3H), 1.89 (d, J=6.8 Hz, 3H), 0.92-0.78 (m, 5H).

### Example 211: Preparation of Compound 211

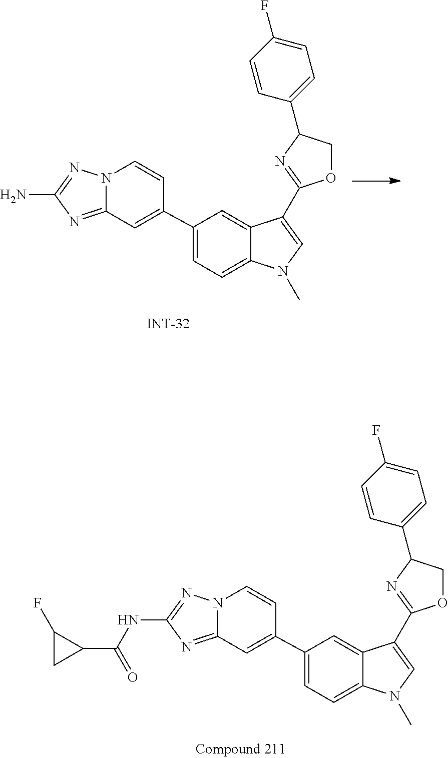 [SMILES: Cn1cc(C2=NC(c3ccc(F)cc3)CO2)c2cc(-c3ccn4nc(N)nc4c3)ccc21 ; "INT-32" ; Cn1cc(C2=NC(c3ccc(F)cc3)CO2)c2cc(-c3ccn4nc(NC(=O)C5CC5F)nc4c3)ccc21]

### Preparation of Compound 211

Compound INT-32 (85 mg, 0.2 mmol) was dissolved in pyridine (1 mL), 2-fluorocyclopropanecarboxylic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 211 with a yield of 64%. MS (ESI, positive ion) m/z: 513.2 [M+H]⁺.

¹H NMR (400 MHz, DMSO-d₆) δ 10.53 (s, 1H), 8.76 (m, 1H), 8.55 (d, J=1.8 Hz, 1H), 8.27 (s, 1H), 7.91 (dd, J=1.9, 0.9 Hz, 1H), 7.76 (s, 1H), 7.69 (d, J=8.6 Hz, 1H), 7.60-7.53 (m, 1H), 7.48 (m, 2H), 7.28-7.18 (m, 2H), 5.54-5.49 (m, 1H), 4.71-4.59 (m, 3H), 3.72 (s, 3H), 2.60-2.55 (m, 1H), 1.42 (m, 1H), 1.21-1.06 (m, 2H).

### Example 212: Preparation of Compound 212

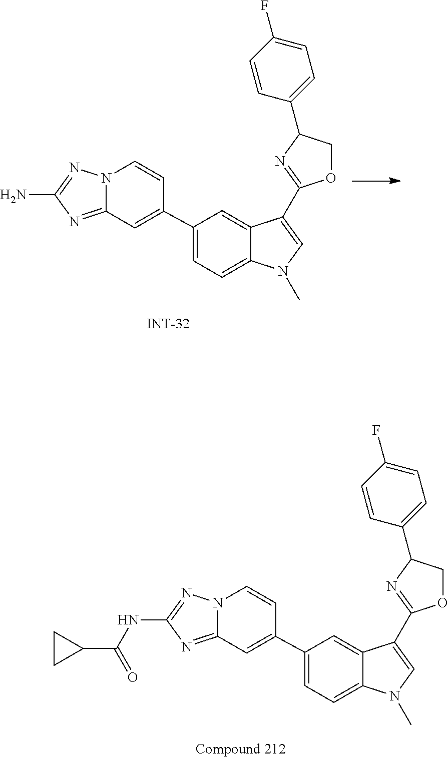 [SMILES: Cn1cc(C2=NC(c3ccc(F)cc3)CO2)c2cc(-c3ccn4nc(N)nc4c3)ccc21 ; "INT-32" ; Cn1cc(C2=NC(c3ccc(F)cc3)CO2)c2cc(-c3ccn4nc(NC(=O)C5CC5)nc4c3)ccc21]

### Preparation of Compound 212

Compound INT-32 (85 mg, 0.2 mmol) was dissolved in pyridine (1 mL), cyclopropanecarboxylic acid (28 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 212 with a yield of 68%. MS (ESI, positive ion) m/z: 495.2 [M+H]⁺.

¹H NMR (400 MHz, DMSO-d₆) δ 10.53 (s, 1H), 8.76 (m, 1H), 8.55 (d, J=1.8 Hz, 1H), 8.27 (s, 1H), 7.91 (dd, J=1.9, 0.9 Hz, 1H), 7.76 (s, 1H), 7.69 (d, J=8.6 Hz, 1H), 7.60-7.53 (m, 1H), 7.48 (m, 2H), 7.28-7.18 (m, 2H), 5.54-5.49 (m, 1H), 4.71-4.59 (m, 2H), 3.72 (s, 3H), 2.60-2.55 (m, 1H), 1.42 (m, 1H), 0.72-0.56 (m, 4H).

### Example 213: Preparation of Compound 213

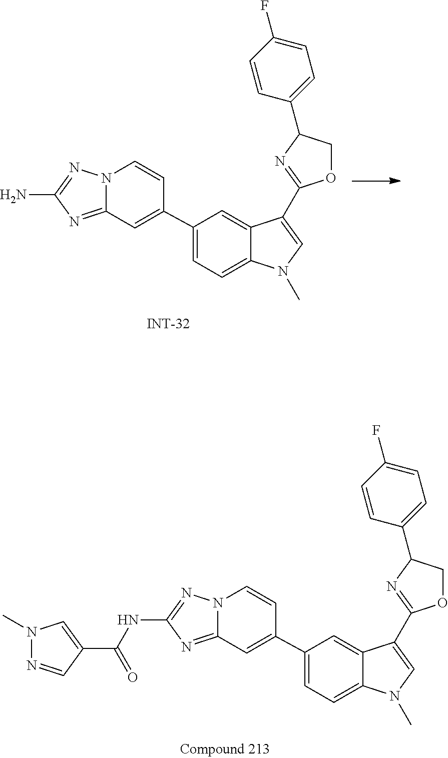 [SMILES: Cn1cc(C2=NC(c3ccc(F)cc3)CO2)c2cc(-c3ccn4nc(N)nc4c3)ccc21 ; "INT-32" ; Cn1cc(C(=O)Nc2nc3cc(-c4ccc5c(c4)c(C4=NC(c6ccc(F)cc6)CO4)cn5C)ccn3n2)cn1]

### Preparation of Compound 213

Compound INT-32 (85 mg, 0.2 mmol) was dissolved in pyridine (1 mL), 1-methylpyrazole-4-carboxylic acid (38 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 213 with a yield of 58%. MS (ESI, positive ion) m/z: 535.2 [M+H]⁺.

¹H NMR (400 MHz, DMSO-d₆) δ 10.53 (s, 1H), 8.76 (m, 2H), 8.55 (d, J=1.8 Hz, 1H), 8.27 (s, 1H), 7.91 (dd, J=1.9, 0.9 Hz, 1H), 7.76 (s, 1H), 7.69 (d, J=8.6 Hz, 1H), 7.60-7.53 (m, 2H), 7.48 (m, 2H), 7.28-7.18 (m, 2H), 5.54-5.49 (m, 1H), 4.71-4.59 (m, 2H), 3.92 (s, 3H), 3.71 (s, 3H).

### Example 214: Preparation of Compound 214

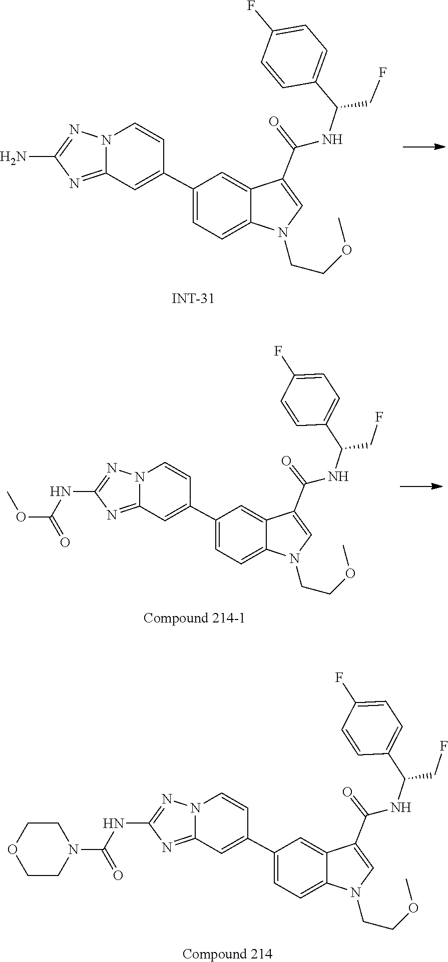 [SMILES: COCCn1cc(C(=O)N[C@@H](CF)c2ccc(F)cc2)c2cc(-c3ccn4nc(N)nc4c3)ccc21 ; "INT-31" ; COCCn1cc(C(=O)N[C@@H](CF)c2ccc(F)cc2)c2cc(-c3ccn4nc(NC(=O)OC)nc4c3)ccc21 ; "Compound 214-1" ; COCCn1cc(C(=O)N[C@@H](CF)c2ccc(F)cc2)c2cc(-c3ccn4nc(NC(=O)N5CCOCC5)nc4c3)ccc21 ; "Compound 214"]

### Preparation of Compound 214-1

Intermediate INT-31 (490 mg, 1 mmol) was dissolved in dichloromethane (0.3 L); methyl chloroformate (112 mg, 1.2 mmol) and N,N-diisopropylethylamine (258 mg, 2.0 mmol) were added, and the mixture was stirred at room temperature for 2 h. Petroleum ether (0.3 L) was added to the reaction mixture for crystallization; the solid was filtered with suction, washed with water, and dried to obtain Compound 214-1 with a yield of 65%. MS (ESI, positive ion) m/z: 549.2 [M+H]⁺.

### Preparation of Compound 214

Compound 214-1 (110 mg, 0.2 mmol) was dissolved in acetonitrile (1 mL); morpholine (36 mg, 0.4 mmol) was added, and the mixture was heated to 80° C. and stirred for 6 h. The reaction mixture was concentrated under reduced pressure and purified by C-18 reverse phase silica gel column (acetonitrile/0.1% aqueous ammonium bicarbonate solution=60%) to obtain Compound 214 with a yield of 58%. MS (ESI, positive ion) m/z: 604.2 [M+H]⁺.

¹H NMR (400 MHz, DMSO-d₆) δ 10.53 (s, 1H), 8.90 (dd, J=7.1, 0.8 Hz, 1H), 8.62 (d, J=8.3 Hz, 1H), 8.55 (d, J=1.8 Hz, 1H), 8.27 (s, 1H), 7.91 (dd, J=1.9, 0.9 Hz, 1H), 7.76 (dd, J=8.7, 1.8 Hz, 1H), 7.69 (d, J=8.6 Hz, 1H), 7.60-7.53 (m, 2H), 7.48 (dd, J=7.2, 2.0 Hz, 1H), 7.28-7.18 (m, 2H), 5.49 (dq, J=15.3, 7.3 Hz, 1H), 4.70-4.60 (m, 3H), 3.64 (m, 6H), 3.43 (s, 3H), 3.27 (s, 2H), 2.58-2.55 (m, 4H).

### Example 215: Preparation of Compound 215

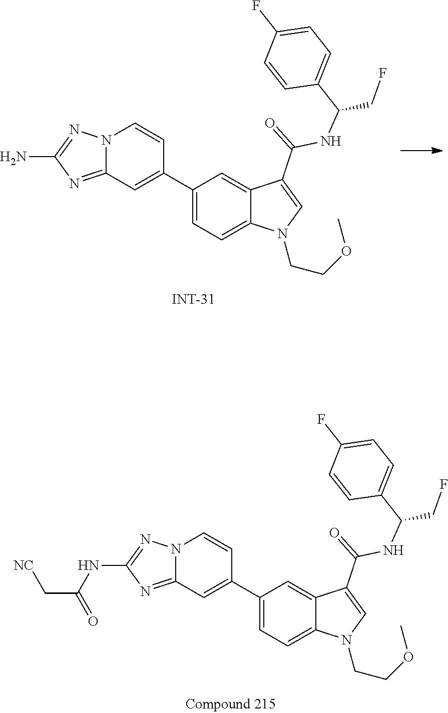 [SMILES: COCCn1cc(C(=O)N[C@@H](CF)c2ccc(F)cc2)c2cc(-c3ccn4nc(N)nc4c3)ccc21 ; "INT-31" ; COCCn1cc(C(=O)N[C@@H](CF)c2ccc(F)cc2)c2cc(-c3ccn4nc(NC(=O)CC#N)nc4c3)ccc21]

### Preparation of Compound 215

Compound INT-31 (85 mg, 0.2 mmol) was dissolved in pyridine (1 mL), cyanoacetic acid (25 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 215 with a yield of 58%. MS (ESI, positive ion) m/z: 558.2 [M+H]⁺.

¹H NMR (400 MHz, DMSO-d₆) δ 10.53 (s, 1H), 8.90 (dd, J=7.1, 0.8 Hz, 1H), 8.62 (d, J=8.3 Hz, 1H), 8.55 (d, J=1.8 Hz, 1H), 8.27 (s, 1H), 7.91 (dd, J=1.9, 0.9 Hz, 1H), 7.76 (dd, J=8.7, 1.8 Hz, 1H), 7.69 (d, J=8.6 Hz, 1H), 7.60-7.53 (m, 2H), 7.48 (dd, J=7.2, 2.0 Hz, 1H), 7.28-7.18 (m, 2H), 5.49 (dq, J=15.3, 7.3 Hz, 1H), 4.70-4.60 (m, 3H), 3.71-3.60 (m, 2H), 3.34 (s, 2H), 3.23 (s, 3H), 3.27 (s, 2H).

### Example 216: Preparation of Compound 216

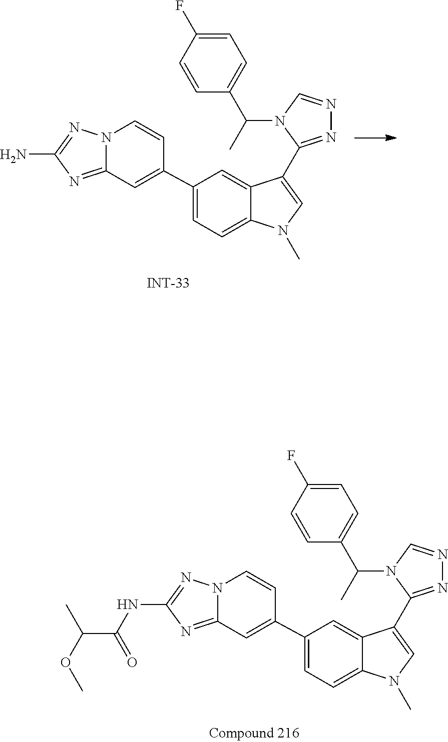 [SMILES: CC(c1ccc(F)cc1)n1cnnc1-c1cn(C)c2ccc(-c3ccn4nc(N)nc4c3)cc12 ; "INT-33" ; COC(C)C(=O)Nc1nc2cc(-c3ccc4c(c3)c(-c3nncn3C(C)c3ccc(F)cc3)cn4C)ccn2n1]

### Preparation of Compound 216

INT-33 (90 mg, 0.2 mmol) was dissolved in pyridine (1 mL), 2-methoxypropanoic acid (31 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 216 with a yield of 66%. MS (ESI, positive ion) m/z: 539.2 [M+H]⁺.

¹H NMR (400 MHz, DMSO-d₆) δ 10.50 (s, 1H), 8.95-8.85 (m, 2H), 8.66 (s, 1H), 8.56-8.50 (m, 1H), 8.03-7.96 (m, 2H), 7.92 (d, J=9.0 Hz, 1H), 7.52 (m, 3H), 7.22-7.12 (m, 2H), 5.26 (p, J=7.2 Hz, 1H), 4.18 (m, 1H), 3.39 (s, 3H), 1.55 (d, J=7.1 Hz, 3H), 1.35 (d, J=6.6 Hz, 3H).

### Example 217: Preparation of Compound 217

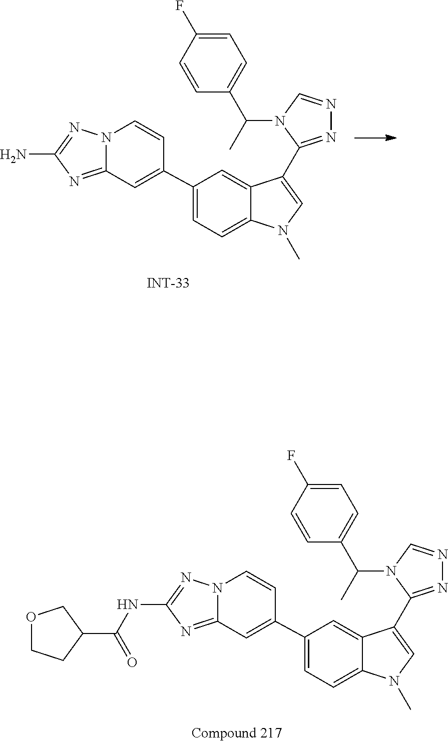 [SMILES: CC(c1ccc(F)cc1)n1cnnc1-c1cn(C)c2ccc(-c3ccn4nc(N)nc4c3)cc12 ; "INT-33" ; CC(c1ccc(F)cc1)n1cnnc1-c1cn(C)c2ccc(-c3ccn4nc(NC(=O)C5CCOC5)nc4c3)cc12]

### Preparation of Compound 217

INT-33 (90 mg, 0.2 mmol) was dissolved in pyridine (1 mL), tetrahydrofuran-3-carboxylic acid (35 mg, 0.3 mmol) and 1-propylphosphonic anhydride (111 mg, 0.351 mmol) were added, and the mixture was heated to 80° C. and stirred for 2 h. The reaction mixture was concentrated under reduced pressure and purified by C-18 reverse phase silica gel column (acetonitrile/0.1% aqueous ammonium bicarbonate solution=60%) to obtain Compound 217 with a yield of 76%. MS (ESI, positive ion) m/z: 551.2 [M+H]⁺.

¹H NMR (400 MHz, DMSO-d₆) δ 10.50 (s, 1H), 8.95-8.85 (m, 2H), 8.66 (s, 1H), 8.56-8.50 (m, 1H), 8.03-7.96 (m, 2H), 7.92 (d, J=9.0 Hz, 1H), 7.52 (m, 3H), 7.22-7.12 (m, 2H), 5.26 (p, J=7.2 Hz, 1H), 4.53 (s, 1H), 4.22 (s, 3H), 4.01 (q, J=6.9 Hz, 1H), 3.84 (td, J=7.6, 6.5 Hz, 1H), 2.30-1.92 (m, 2H), 1.97-1.82 (m, 2H), 1.55 (d, J=7.1 Hz, 3H).

### Example 218: Preparation of Compound 218

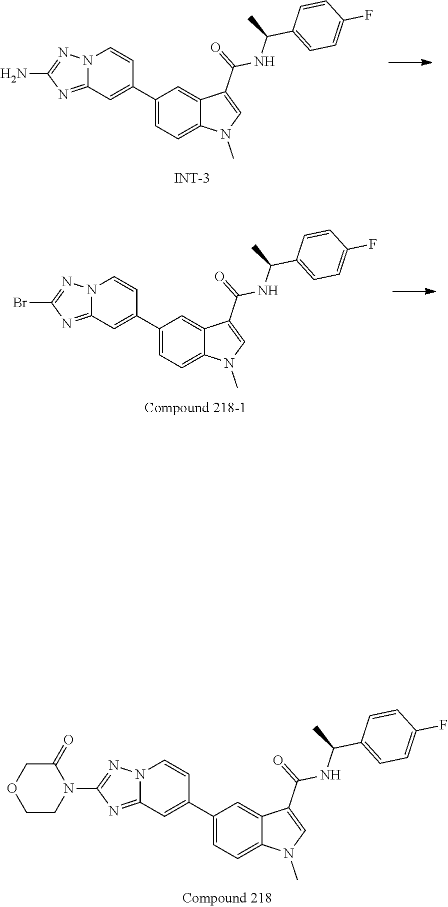 [SMILES: C[C@H](NC(=O)c1cn(C)c2ccc(-c3ccn4nc(N)nc4c3)cc12)c1ccc(F)cc1 ; "INT-3" ; C[C@H](NC(=O)c1cn(C)c2ccc(-c3ccn4nc(Br)nc4c3)cc12)c1ccc(F)cc1 ; "Compound 218-1" ; C[C@H](NC(=O)c1cn(C)c2ccc(-c3ccn4nc(N5CCOCC5=O)nc4c3)cc12)c1ccc(F)cc1]

### Preparation of Compound 218-1

Under an argon atmosphere, Intermediate INT-3 (2 g, 4.7 mmol) and copper bromide (3 g, 14.1 mmol) were dissolved in acetonitrile (40 mL), and the mixture was heated to reflux. Tert-butyl nitrite (0.63 g, 6.1 mmol) was added dropwise. After the addition was completed, the reaction mixture was stirred for reaction for 1 h. The reaction mixture was concentrated under reduced pressure and purified by silica gel column (dichloromethane/methanol=40/1) to obtain Compound 218-1 with a yield of 35%. MS (ESI, positive ion) m/z: 492.2 [M+H]⁺.

### Preparation of Compound 218

Under an argon atmosphere, 3-morpholinone (155 mg, 1.5 mmol) was dissolved in ethylene glycol dimethyl ether (10 mL), sodium hydride (60 mg, 1.5 mmol, 60%) was added at 0° C.-5° C., and the mixture was stirred at this temperature for 0.5 h. Intermediate INT-2 (246 mg, 0.5 mmol) and PdCl₂(dppf)₂ (25 mg) were added, and the mixture was heated to reflux and stirred for reaction for 2 h. The reaction mixture was concentrated under reduced pressure and purified by silica gel column (dichloromethane/methanol=20/1) to obtain Compound 218 with a yield of 21%. MS (ESI, positive ion) m/z: 513.2 [M+H]⁺.

¹H NMR (400 MHz, DMSO-d₆) δ 8.96 (d, J=7.1 Hz, 1H), 8.56 (d, J=1.8 Hz, 1H), 8.37 (d, J=8.0 Hz, 1H), 8.23 (s, 1H), 8.01 (d, J=1.9 Hz, 1H), 7.75 (dd, J=8.7, 1.8 Hz, 1H), 7.68 (d, J=8.6 Hz, 1H), 7.55 (dd, J=7.2, 1.9 Hz, 1H), 7.52-7.43 (m, 2H), 7.22-7.12 (m, 2H), 5.23 (p, J=7.2 Hz, 1H), 4.32 (s, 2H), 4.09-3.95 (m, 4H), 3.92 (s, 3H), 1.51 (d, J=7.1 Hz, 3H).

### Example 219: Preparation of Compound 219

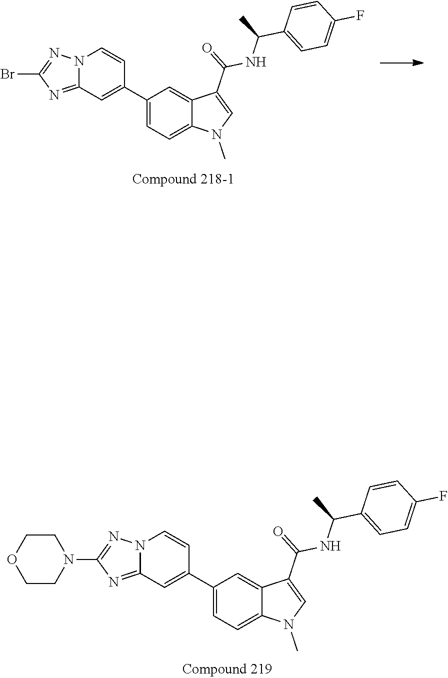 [SMILES: C[C@H](NC(=O)c1cn(C)c2ccc(-c3ccn4nc(Br)nc4c3)cc12)c1ccc(F)cc1 ; "Compound 218-1" ; C[C@H](NC(=O)c1cn(C)c2ccc(-c3ccn4nc(N5CCOCC5)nc4c3)cc12)c1ccc(F)cc1]

### Preparation of Compound 219

Under an argon atmosphere, morpholine (87 mg, 1 mmol), Compound 218-1 (246 mg, 0.5 mmol), Pd₂(dba)₃ (20 mg), and 4,5-bis(diphenylphosphino)-9,9-dimethylxanthene (20 mg) were dissolved in 1,4-dioxane (3 mL), cesium carbonate (325 mg, 1 mmol, 60%) was added, and the mixture was heated to reflux and stirred for reaction overnight. The reaction mixture was concentrated under reduced pressure and purified by silica gel column (dichloromethane/methanol=30/1) to obtain Compound 219 with a yield of 24%. MS (ESI, positive ion) m/z: 499.2 [M+H]⁺.

¹H NMR (400 MHz, DMSO-d₆) δ 8.69 (d, J=7.0 Hz, 1H), 8.52-8.48 (m, 1H), 8.34 (d, J=7.9 Hz, 1H), 8.21 (d, J=6.7 Hz, 1H), 7.73-7.62 (m, 3H), 7.49-7.45 (m, 2H), 7.29 (dd, J=7.1, 2.0 Hz, 1H), 7.20-7.14 (m, 2H), 5.22 (t, J=7.4 Hz, 1H), 3.91 (d, J=4.6 Hz, 3H), 3.74 (t, J=4.8 Hz, 4H), 3.50 (t, J=4.8 Hz, 4H), 1.50 (d, J=7.1 Hz, 3H).

### Test Example 1: In Vitro Kinase Assay and Assay for Protection Against Necroptosis for Compound in the Present Invention

### In Vitro RIPK1 Kinase Assay:

This assay was completed using the Kinase Profiler service provided by Eurofins. The experimental method was as follows: The small molecule compound to be tested (0.001-10 M) and the protein kinase to be tested were incubated with a buffer containing a substrate, 10 mM magnesium acetate, and [γ-³³P-ATP]. The reaction was initiated by adding a Mg\ATPmix. After incubation at room temperature for a period of time, a 3% phosphate solution was added to the buffer to terminate the reaction. Subsequently, 10 μL of the reaction mixture was quantitatively pipetted and dropped onto P30 filter paper. The paper was washed three times with 75 mM phosphate solution and once with methanol. After drying, scintillation fluid was added for scinticounting. The inhibitory activity of the compound was expressed as the half-maximal inhibitory concentration (IC₅₀). The IC₅₀ values were derived by fitting the inhibition rates corresponding to the respective concentration gradients. The in vitro MLK2/3 kinase assay utilized the ADP-Glo method provided by ICE Bioscience INC. A drug screening system based on MLK2/3 kinase was employed to evaluate the inhibiting ability of small molecule compounds against kinase activity. The experimental method is as follows:

### ADP-Glo Method

1. 2×ATP/substrate solution and 2× kinase solution were prepared using kinase reaction buffer.
2. 40 nL of the compound diluent was transferred into a 384-well plate using an Echo 655. After centrifugation, 2 μL of 2× kinase solution was added to the 384-well plate, centrifuged at 1000 rpm for 1 min, and incubated at 25° C. for 10 min;
3. 2 μL of 2× substrate and ATP solution was added into the 384-well plate, centrifuged at 1000 rpm for 1 min, and incubated at 25° C. for 60 min.
4. 4 μL of ADP-Glo was transferred to a 384-well plate, centrifuged at 1000 rpm for 1 min, and incubated at 25° C. for 40 min.
5. 8 μL of test solution was transferred to a 384-well plate, centrifuged at 1000 rpm for 1 min, and incubated at 25° C. for 40 min.
6. The luminescence signal was read using a multifunctional microplate reader.
7. Data Analysis
The negative control reading was set at 0% inhibition rate and the positive control reading was set at 100% inhibition rate to calculate the inhibition rate of each test solution.

$$\% Compound Inhibition Rate = ⌊ 1 - \frac{{Data}_{Compound} - {\overline{Data}}_{Positive Control}}{{\overline{Data}}_{Negative Control} - {\overline{Data}}_{Positive Control}} ⌋ * 100 \%$$

D̅a̅t̅a̅_{Positive control}: Mean value of positive control wells
D̅a̅t̅a̅_{Negative Control}: Mean value of negative control wells

The IC₅₀ (half inhibitory concentration) of a compound was obtained using the following non-linear fitting formula:

Y=Bottom+(Top-Bottom)/(1+10\hat{ }((Log IC₅₀−X)*HillSlope))
X: Compound concentration log value
Y: Compound inhibition rate (% inh)

Equation for Z′ factor calculation:

Z′=1−3(SDmin+SDmax)/(AVEmax−AVEmin)
wherein:
Min is the positive control Data, and Max is the negative control DMSO Data.
SD is the standard deviation, and AVE is the average.
   In Vitro Assay for Protection of HT-29 Cells from Necroptosis:

### 1) Experimental Materials

DMEM medium was purchased from Gibco; penicillin-streptomycin was purchased from Hyclone; TNFα was purchased from Peprotech; Smac mimetic and Z-VAD-FMK were purchased from Selleck; and CCK-8 was purchased from Medchem Express.

### 2) Experimental Methods

HT-29 cells (colon cancer cells) were cultured in DMEM+10% FBS+penicillin/streptomycin. For the experiment, HT-29 cells in the logarithmic phase were collected and seeded into 96-well plates at a specific density (typically 8×10³ cells/well for adherent cells). The cells were cultured overnight in an incubator at 37° C. with 5% CO₂. On the following day, the test compound was diluted with culture medium to the corresponding concentrations and added into the 96-well plate, with 3 replicate wells for each sample. A solvent control group and a blank control group containing only culture medium were set simultaneously. The spiked cells were induced with a combination of TNFα/Smac mimetic/Z-VAD-FMK for 24 h. Subsequently, 10 μL of CCK8 solution was added to each well, and the cells were incubated in an incubator for 1-3 h. Absorbance was measured at 495 nm using a microplate reader, and the cell necrosis protection rate of the drug was calculated using the following formula:

Cell necrosis protection rate=[(X−C₀)/(C−C₀)]×100%
where C, C₀, and X represent the average absorbance values of the solvent control group, the blank control group, and the drug treatment group, respectively. Finally, a cell viability curve was fitted using Graphpad Prism 5.0 software, and the EC₅₀ value of the test compound for inhibiting necroptosis was calculated.

The results are shown in Table 1, wherein “+++++” represents IC₅₀ or EC₅₀<0.001 μM; “++++” represents 0.01 μM>IC₅₀ or EC₅₀≥0.001 μM; “+++” represents 0.1 μM>IC₅₀ or EC₅₀≥0.01 μM; “++” represents 1 μM>IC₅₀ or EC₅₀≥0.1 μM; and “+” represents 10 μM>IC₅₀ or EC₅₀≥1 μM.

TABLE 1
Inhibitory Activity of Compound in the
Present Invention Against RIPK1 Kinase

| Compound No. | RIPK1(IC₅₀) |
|---|---|
| 3 | +++ |
| 6 | +++ |
| 8 | +++ |
| 9 | +++ |
| 14 | +++ |
| 18 | +++ |
| 27 | +++ |
| 29 | +++ |
| 32 | +++ |
| 38 | +++ |
| 39 | +++ |
| 40 | +++ |
| 42 | +++ |
| 46 | +++ |
| 51 | +++ |
| 59 | +++ |
| 60 | +++ |
| 68 | +++ |
| 71 | +++ |
| 73 | +++ |
| 74 | +++ |
| 75 | ++++ |
| 106 | +++ |
| 121 | +++ |
| 122 | ++ |
| 124 | +++ |
| 127 | ++ |
| 130 | +++ |
| 138 | +++ |
| 156 | +++ |
| 161 | ++++ |
| 175 | +++ |
| 182 | ++ |
| 196 | +++ |
| 210 | + |

TABLE 2
Inhibitory Activity of Compound in the
Present Invention Against MLK2/3 Kinase

| Compound No. | MLK2/3 (IC₅₀) |
|---|---|
| 73 | +++ |
| 74 | +++ |
| 75 | +++ |

TABLE 3
Protective Activity of Compound in the
Present Invention Against Necroptosis

| Compound No. | HT-29 (EC₅₀) |
|---|---|
| 1 | +++ |
| 2 | − |
| 3 | +++ |
| 4 | +++ |
| 5 | +++++ |
| 6 | +++++ |
| 7 | +++++ |
| 8 | +++++ |
| 9 | +++++ |
| 10 | ++++ |
| 11 | ++++ |
| 12 | +++++ |
| 13 | +++++ |
| 14 | +++++ |
| 15 | +++++ |
| 16 | +++ |
| 17 | +++++ |
| 18 | +++++ |
| 19 | +++++ |
| 20 | +++++ |
| 21 | +++++ |
| 22 | ++++ |
| 23 | +++++ |
| 24 | +++++ |
| 25 | +++++ |
| 26 | +++++ |
| 27 | +++++ |
| 28 | ++++ |
| 29 | +++++ |
| 30 | +++++ |
| 31 | +++ |
| 32 | +++++ |
| 33 | + |
| 34 | +++++ |
| 35 | +++++ |
| 36 | +++++ |
| 37 | ++++ |
| 38 | +++++ |
| 39 | +++++ |
| 40 | +++++ |
| 41 | +++++ |
| 42 | +++++ |
| 43 | +++++ |
| 44 | ++++ |
| 45 | +++++ |
| 46 | +++++ |
| 47 | +++++ |
| 48 | +++ |
| 49 | ++++ |
| 50 | +++++ |
| 51 | +++++ |
| 52 | +++++ |
| 53 | +++++ |
| 54 | − |
| 55 | − |
| 56 | − |
| 57 | +++ |
| 58 | +++++ |
| 59 | +++++ |
| 60 | +++++ |
| 61 | ++++ |
| 62 | +++++ |
| 63 | + |
| 64 | +++++ |
| 65 | +++++ |
| 66 | +++++ |
| 67 | +++++ |
| 68 | +++++ |
| 69 | +++++ |
| 70 | +++++ |
| 71 | +++++ |
| 72 | +++++ |
| 73 | +++++ |
| 74 | +++++ |
| 75 | +++++ |
| 76 | +++++ |
| 77 | +++++ |
| 78 | +++++ |
| 79 | +++++ |
| 80 | +++++ |
| 81 | +++++ |
| 82 | +++++ |
| 83 | +++++ |
| 84 | +++++ |
| 85 | +++++ |
| 86 | +++++ |
| 87 | +++++ |
| 88 | ++++ |
| 89 | − |
| 90 | + |
| 91 | + |
| 92 | ++ |
| 93 | ++ |
| 94 | +++++ |
| 95 | +++++ |
| 96 | +++++ |
| 97 | +++++ |
| 98 | +++++ |
| 99 | +++++ |
| 100 | +++++ |
| 101 | +++++ |
| 102 | +++++ |
| 103 | +++++ |
| 104 | + |
| 105 | +++ |
| 106 | +++++ |
| 107 | +++++ |
| 108 | +++++ |
| 109 | ++++ |
| 110 | +++++ |
| 111 | ++++ |
| 112 | +++++ |
| 113 | ++++ |
| 114 | +++++ |
| 115 | +++++ |
| 116 | +++++ |
| 117 | +++++ |
| 118 | +++++ |
| 119 | +++++ |
| 120 | +++++ |
| 121 | +++++ |
| 122 | ++ |
| 123 | ++++ |
| 124 | ++++ |
| 125 | + |
| 126 | − |
| 127 | + |
| 128 | − |
| 129 | − |
| 130 | ++++ |
| 131 | − |
| 132 | ++ |
| 133 | − |
| 134 | − |
| 135 | − |
| 136 | +++ |
| 137 | +++ |
| 138 | − |
| 139 | ++++ |
| 140 | − |
| 141 | ++++ |
| 142 | +++ |
| 143 | ++++ |
| 144 | − |
| 145 | ++++ |
| 146 | +++ |
| 147 | ++++ |
| 148 | +++ |
| 149 | +++++ |
| 150 | +++++ |
| 151 | +++++ |
| 152 | − |
| 153 | +++++ |
| 154 | +++++ |
| 155 | +++++ |
| 156 | +++++ |
| 157 | +++++ |
| 158 | + |
| 159 | +++++ |
| 160 | +++++ |
| 161 | +++++ |
| 162 | +++++ |
| 163 | +++++ |
| 164 | +++++ |
| 165 | +++++ |
| 166 | +++++ |
| 167 | +++++ |
| 168 | +++++ |
| 169 | +++++ |
| 170 | +++++ |
| 171 | ++++ |
| 172 | ++++ |
| 173 | +++++ |
| 174 | +++++ |
| 175 | +++++ |
| 176 | +++++ |
| 177 | +++ |
| 178 | ++ |
| 179 | ++ |
| 180 | + |
| 181 | + |
| 182 | +++ |
| 183 | +++++ |
| 184 | ++ |
| 185 | +++++ |
| 186 | +++++ |
| 187 | +++++ |
| 188 | +++++ |
| 189 | +++++ |
| 190 | +++++ |
| 191 | +++++ |
| 192 | +++++ |
| 193 | +++++ |
| 194 | − |
| 195 | +++++ |
| 196 | +++++ |
| 197 | +++++ |
| 198 | ++ |
| 199 | +++++ |
| 200 | +++++ |
| 201 | − |
| 202 | − |
| 203 | − |
| 204 | +++ |
| 205 | ++++ |
| 206 | + |
| 207 | +++++ |
| 208 | ++ |
| 209 | ++ |
| 210 | ++ |
| 211 | +++ |
| 212 | +++ |
| 213 | +++ |
| 214 | +++ |
| 215 | +++ |
| 216 | +++ |
| 217 | +++ |
| 218 | +++ |
| 219 | ++++ |

Note:
“−” indicates not tested.

The experimental results in Tables 1 and 2 showed that the exemplary compound in the present invention has significant inhibitory activities against both RIPK1 kinase and MLK2/3 kinase, with IC₅₀ value for RIPK1 kinase activity inhibition being less than 1 WM, less than 0.1 WM, or even less than 0.01 μM. The IC₅₀ value for MLK2/3 kinase activity inhibition was less than 1 μM. Table 3 further showed that the compound in the present invention, having inhibitory activity against RIPK1 and/or MLK2/3 kinases, also exhibits significant protection of HT-29 cells from necroptosis.

The aforementioned description of specific exemplary embodiments in the present invention is presented for purposes of explanation and illustration. These descriptions are not intended to limit the present invention to the precise forms disclosed, and obviously, many modifications and variations can be made in light of the above teachings. The exemplary embodiments are chosen and described in order to explain the specific principles of the present invention and its practical application, thereby enabling those skilled persons in the art to implement and utilize various exemplary embodiments in the present invention with various alterations and modifications. The scope of the present invention is intended to be defined by the claims and their equivalents.

1. Compound of formula (I), pharmaceutically acceptable salt, solvate or stereoisomer thereof: 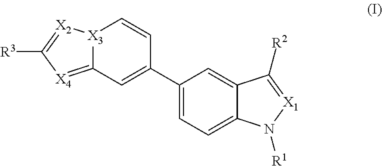
wherein,
X1 is selected from N or CH;
X2. X3 and X4 are each independently selected from N or CH, provided that at least one of X1, X2 and X3 is N;
R¹ is selected from C₁₋₆ alkyl, C₁₋₆ alkoxy, —C₁₋₆ alkyl-C₁₋₆ alkoxy or C₃₋₆ cycloalkyl, wherein the said C₁₋₆ alkyl is optionally substituted with halogen;
R² is selected from 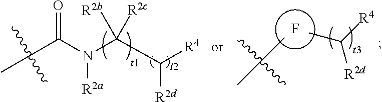
wherein, R⁴ is selected from C₁₋₆ alkyl, C₅₋₈ aryl, 8- to 12-membered partially unsaturated bicyclic group and 5- or 6-membered monocyclic heteroaryl, wherein the said C₅₋₈ aryl, 8- to 12-membered partially unsaturated bicyclic group and 5- or 6-membered monocyclic heteroaryl are optionally substituted with one or more substituents selected from R⁴¹;
R⁴¹ is selected from halogen, cyano, —NR^{4a}R^{4b}, C₁₋₆ alkyl and C₃₋₆ cycloalkyl;
Ring F is selected from 5- or 6-membered monocyclic heteroaryl and 4- to 8-membered nitrogen-containing partially unsaturated heterocyclyl;
R^{2a}. R^{2b}, R^{2c} and R^{2d} are each independently selected from hydrogen, deuterium, halogen, C₁₋₆ alkyl and C₃₋₆ cycloalkyl, wherein the said C₁₋₆ alkyl and C₃₋₆ cycloalkyl are optionally substituted with one or more halogen or hydroxyl;
Alternatively, R^{2b} and R^{2c}, together with the carbon atom to which they are attached, form C₃₋₆ cycloalkyl or 4- to 12-membered heterocycloalkyl;
Alternatively, R^{2a} and R^{2b}, together with the carbon atom and nitrogen atom to which they are attached, form 4- to 12-membered nitrogen-containing heterocycloalkyl or 4- to 8-membered nitrogen-containing partially unsaturated heterocyclyl;
Alternatively, R^{2b} and R^{2d}, together with the carbon atom to which they are attached, form C₃₋₆ cycloalkyl or 4- to 12-membered heterocycloalkyl;
The said C₃₋₆ cycloalkyl, 4- to 12-membered heterocycloalkyl, 4- to 12-membered nitrogen-containing heterocycloalkyl or 4- to 8-membered nitrogen-containing partially unsaturated heterocyclyl is optionally substituted with one or more substituents selected from halogen;
t1. t2 and t3 are each independently selected from integers of 0 to 6;
R³ is selected from 4- to 12-membered heterocycloalkyl, —NH—C₁₋₆ alkyl, —NH-4- to 12-membered heterocycloalkyl or —NH—C(O)R⁵, wherein the said C₁₋₆ alkyl or 4- to 12-membered heterocycloalkyl is optionally substituted with one or more C₃₋₆ cycloalkyl;
R⁵ is selected from hydrogen, deuterium, C₁₋₆ alkyl, C₁₋₆ alkoxy, C₃₋₆ cycloalkyl, 4- to 12-membered heterocycloalkyl, 5- or 6-membered heteroaryl, —NR^{5a}R^{5b}, —C(O)O—C₁₋₆ alkyl, —C(O)NR^{5a}R^{5b}, —NR^{5a}R^{5b}—C(O)O—C₁₋₆ alkyl, —C(O)-4- to 12-membered heterocycloalkyl, —C₁₋₆ alkyl-(OCH₂CH₂)ₘOCH₃, —C₁₋₆ alkyl-NR^{5a}R^{5b}, —C₁₋₆ alkyl-NH—C₁₋₆ alkyl-O—C₁₋₆ alkyl, —C₁₋₆ alkyl-4- to 12-membered heterocycloalkyl, —C₁₋₆ alkyl-NH-4- to 12-membered heterocycloalkyl, —C₁₋₆ alkyl-NH—C₁₋₆ alkyl-4- to 12-membered heterocycloalkyl, or —C1-6 alkyl-5- or 6-membered heteroaryl, wherein the said C₁₋₆ alkyl, C₁₋₆ alkoxy, C₃₋₆ cycloalkyl, 4- to 12-membered heterocycloalkyl, and 5- or 6-membered heteroaryl are optionally substituted with one or more substituents selected from deuterium, halogen, cyano, oxo, hydroxyl, carboxyl, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl, or 4- to 12-membered heterocycloalkyl, wherein two hydrogen atoms on an optional carbon atom of the said C₁₋₆ alkyl are substituted to form a C₃₋₆ cycloalkyl;
m is an integer selected from 1 to 4;
R^{4a}. R^{4b}, R^{5a} and R^{5b} are each independently selected from hydrogen, halogen, C₁₋₆ alkyl or C₁₋₆ alkoxy, wherein the said C₁₋₆ alkyl is optionally substituted with one or more substituents selected from halogen, hydroxyl or C₃₋₆ cycloalkyl.

2. The compound according to claim 1, wherein R¹ is selected from C₁₋₆ alkyl, C₁₋₆ alkoxy, —C₁₋₆ alkyl-C₁₋₆ alkoxy, or C₃₋₆ cycloalkyl;
preferably, R¹ is selected from C₁₋₆ alkyl or —C₁₋₃ alkyl-C₁₋₃ alkoxy;
preferably, R¹ is selected from methyl, ethyl or —CH₂CH₂OCH₃.

3. The compound according to claim 1 or 2, wherein R² is selected from 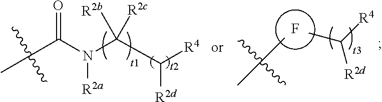
R⁴ is selected from C₁₋₆ alkyl, 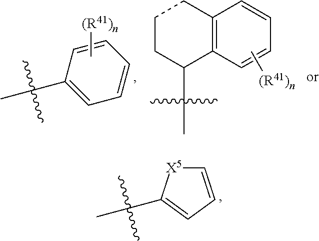
wherein the said R⁴¹ is as defined for the compound of formula (I); X⁵ is selected from N, O or S;
preferably, R⁴¹ is selected from halogen, cyano or amino;
preferably, R⁴¹ is selected from fluoro, chloro or cyano;
preferably, ring F is selected from a 5- or 6-membered monocyclic heteroaryl or a 4- to 8-membered nitrogen-containing partially unsaturated heterocyclyl, wherein the said 5- or 6-membered monocyclic heteroaryl or 4- to 8-membered nitrogen-containing partially unsaturated heterocyclyl optionally contains 1, 2 or 3 heteroatoms selected from N and O as ring atoms;
preferably, ring F is selected from triazolyl or dihydrooxazolyl;
preferably, R^{2a}, R^{2b}, R^{2c} and R^{2d} are each independently selected from hydrogen, deuterium, halogen, C₁₋₆ alkyl, halo-substituted C₁₋₆ alkyl or hydroxy-substituted C₁₋₆ alkyl; more preferably selected from hydrogen, deuterium, halogen, methyl, ethyl, fluoroethyl, difluoroethyl or hydroxymethyl;
preferably, R^{2a} is selected from hydrogen, deuterium or methyl;
preferably, R^{2b}, R^{2c} and R^{2d} are each independently selected from methyl, ethyl, fluoroethyl, difluoroethyl or hydroxymethyl;
preferably, R^{2b} and R^{2c}, together with the carbon atom to which they are attached, form C₃₋₆ cycloalkyl or 4- to 12-membered heterocycloalkyl, wherein the said 4- to 12-membered heterocycloalkyl is preferably 4- to 6-membered oxygen-containing heterocycloalkyl, more preferably oxetanyl;
Alternatively, R^{2a} and R^{2b}, together with the carbon atom and nitrogen atom to which they are attached, form a 4- to 12-membered nitrogen-containing heterocycloalkyl or a 4- to 8-membered nitrogen-containing partially unsaturated heterocyclyl, wherein the said 4- to 12-membered nitrogen-containing heterocycloalkyl is preferably azetidinyl or morpholinyl; the said 4- to 8-membered nitrogen-containing partially unsaturated heterocyclyl is preferably dihydropyrazolyl, and the said azetidinyl is optionally substituted with one or more halogens;
Alternatively, R^{2b}, R^{2d} and the carbon atom to which they are attached together form C₃₋₆ cycloalkyl;
preferably, R2 is selected from

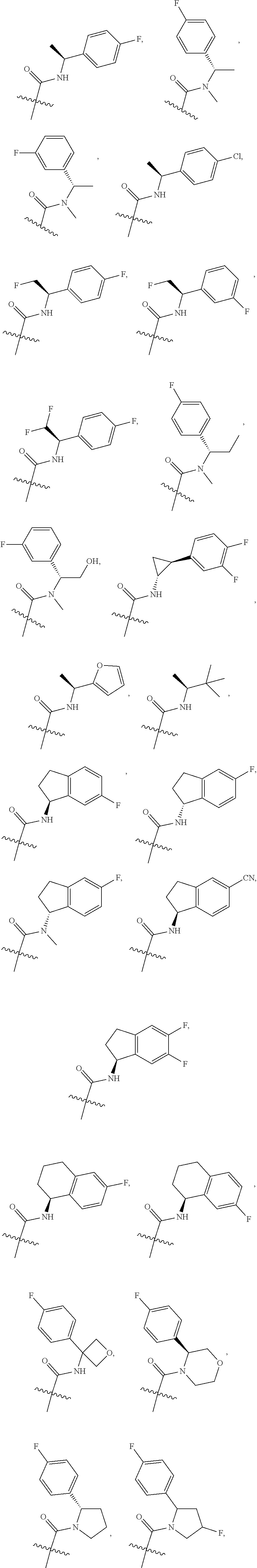

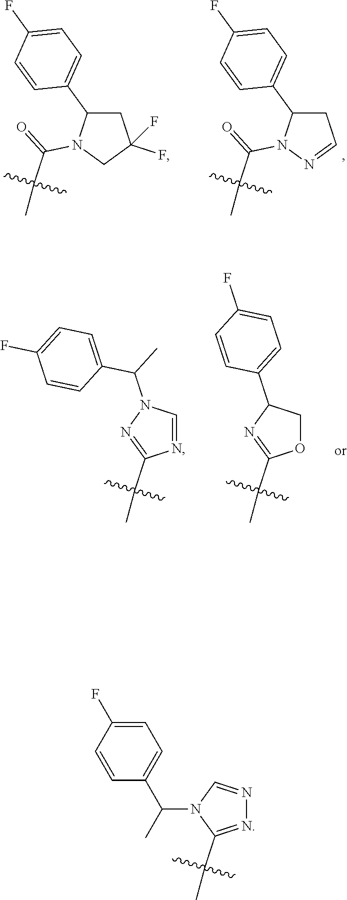 [SMILES: *C(=O)N1CC(F)(F)CC1c1ccc(F)cc1 ; *C(=O)N1N=CCC1c1ccc(F)cc1 ; *c1ncn(C(C)c2ccc(F)cc2)n1 ; *C1=NC(c2ccc(F)cc2)CO1 ; *c1nncn1C(C)c1ccc(F)cc1]

4. The compound according to any one of claims 1 to 3, wherein R³ is selected from 4- to 6-membered heterocycloalkyl, —NH—C₁₋₆ alkyl, —NH-4- to 12-membered heterocycloalkyl or —NH—C(O)R⁵, wherein the said C₁₋₆ alkyl is optionally substituted with one or more C₃₋₆ cycloalkyl, and the said 4- to 6-membered heterocycloalkyl is optionally substituted with one or more oxo;
preferably, R³ is selected from morpholinyl or oxomorpholinyl;
preferably, R³ is selected from —NH-isopropyl, —NH—CH₂-cyclopropyl or —NH-tetrahydrofuran-3-yl;
preferably, R⁵ is selected from methyl, ethoxy, —CH₂CH₂COOH, —CH₂Cl, —CH₂CN, —CH₂OCH₃, —CH(CH₃)OCH₃, 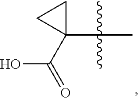 [SMILES: *C1(C(=O)O)CC1]
—C(O)OCH₂CH₃, —C(O)NH₂, —CH₂(OCH₂CH₂)₂OCH₃, —CH₂(OCH₂CH₂)₃OCH₃, 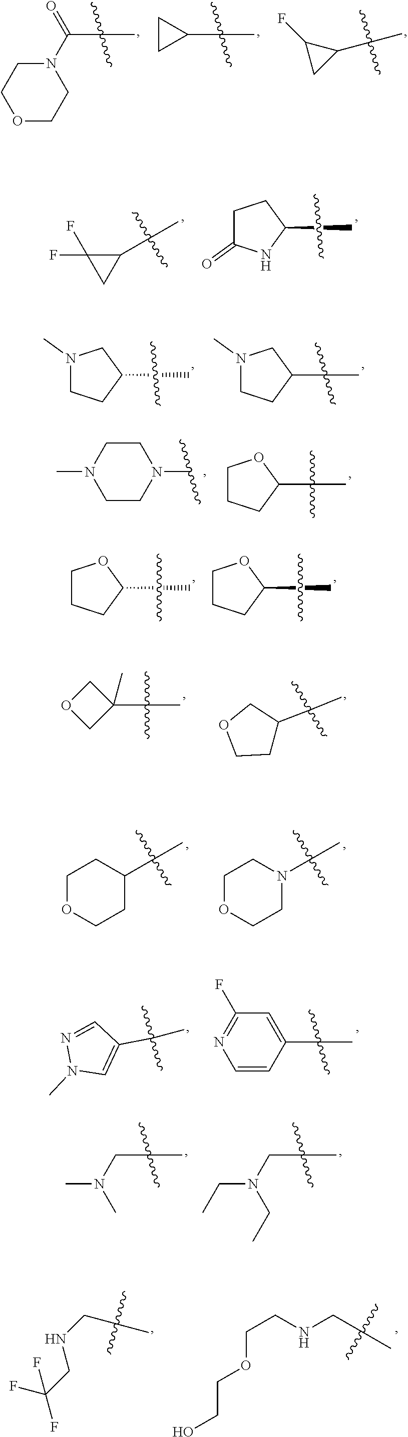 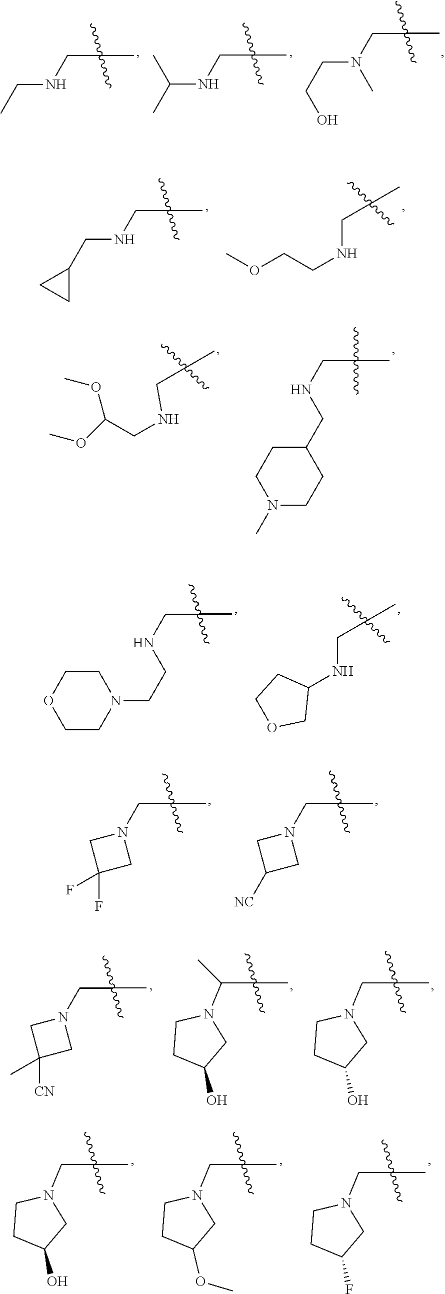 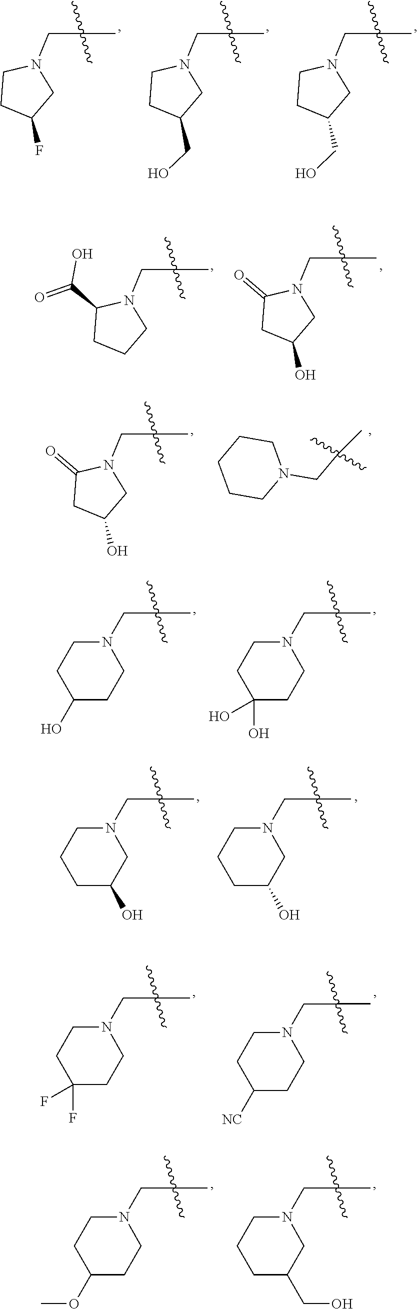 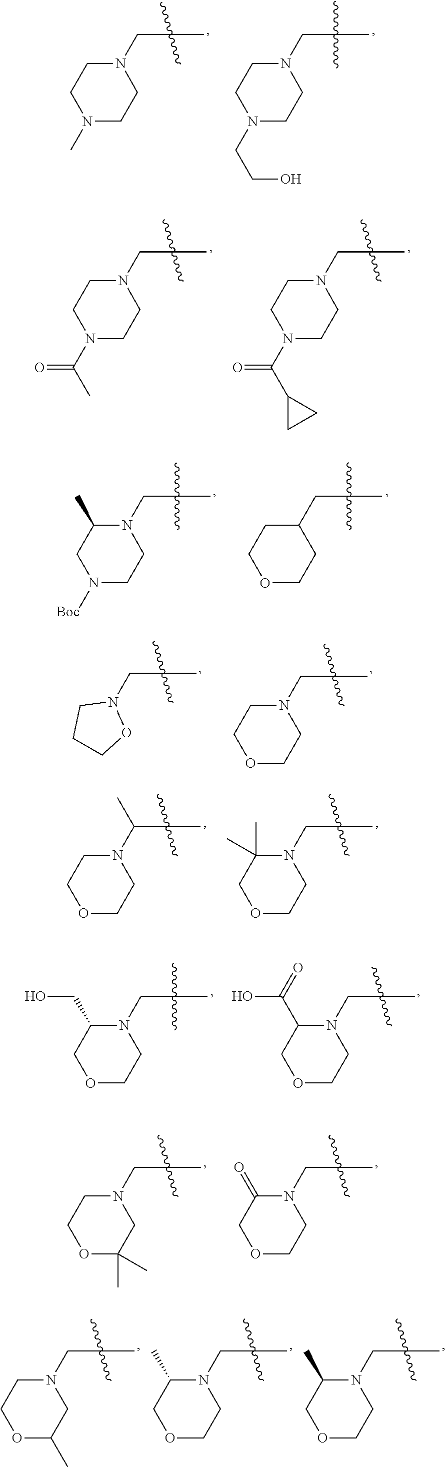 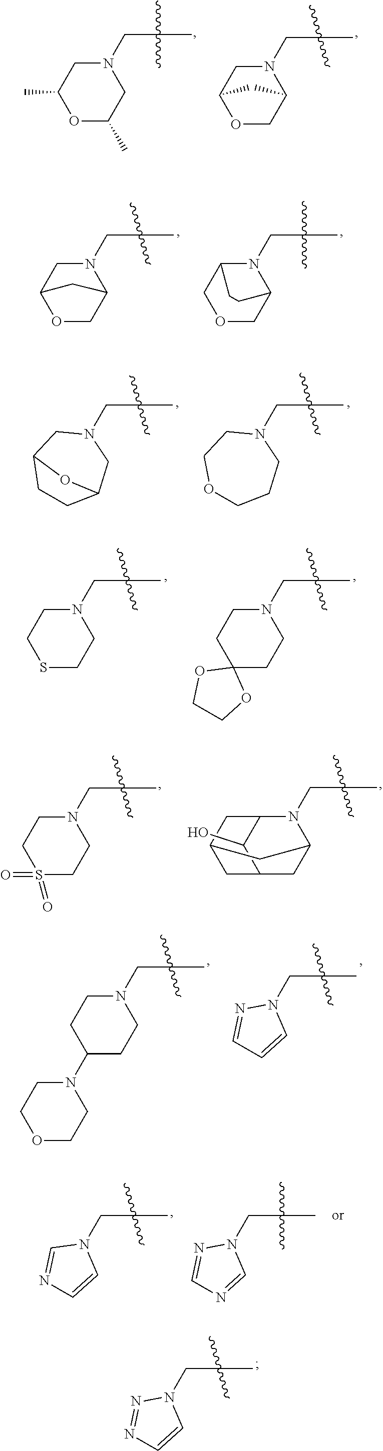
preferably, R³ is selected from: 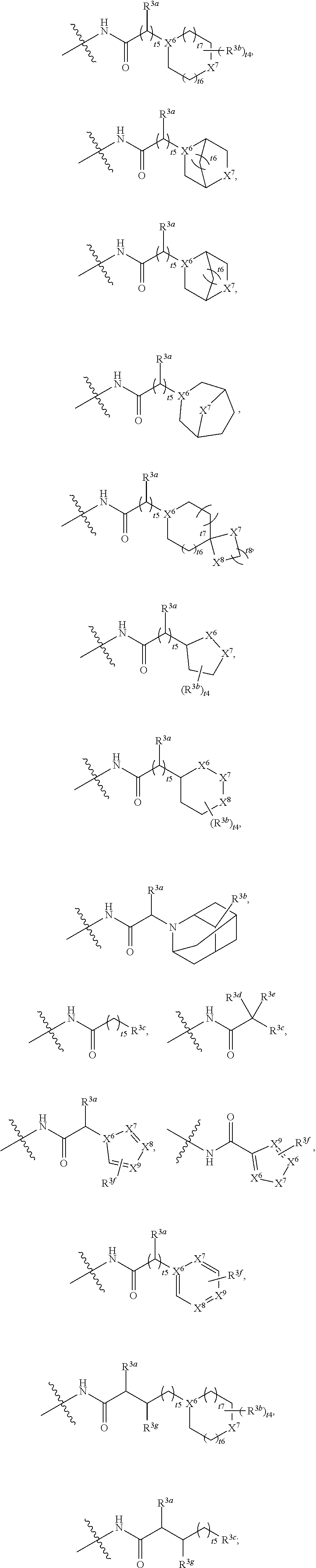
preferably, R³ is selected from: 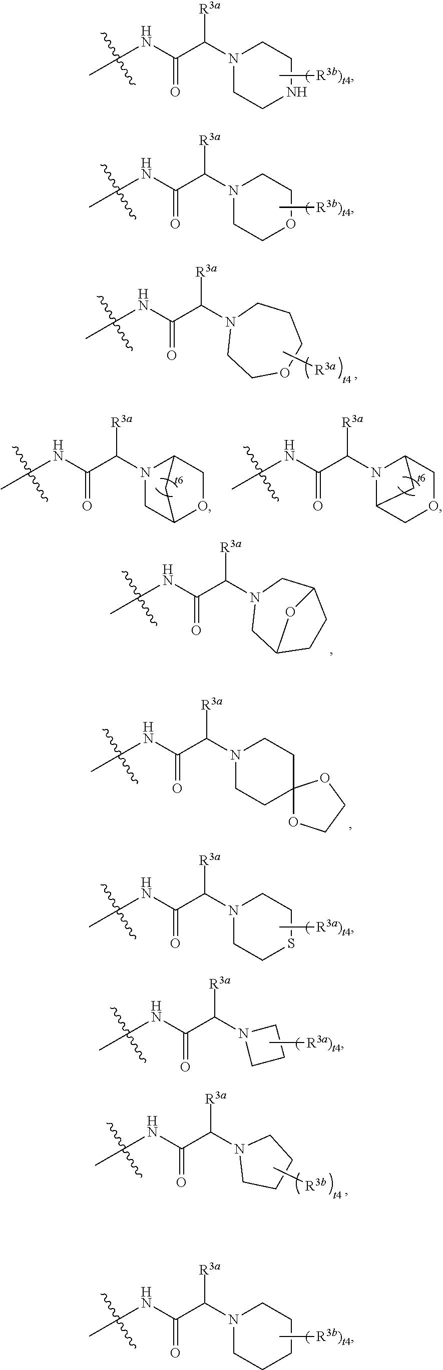 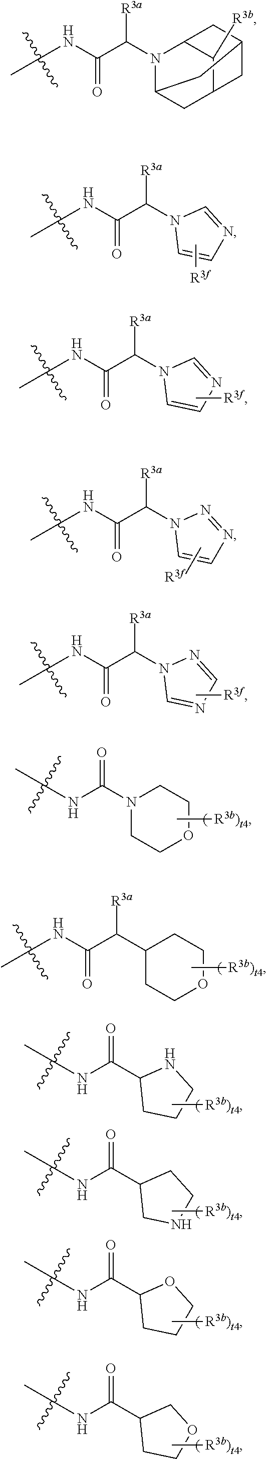 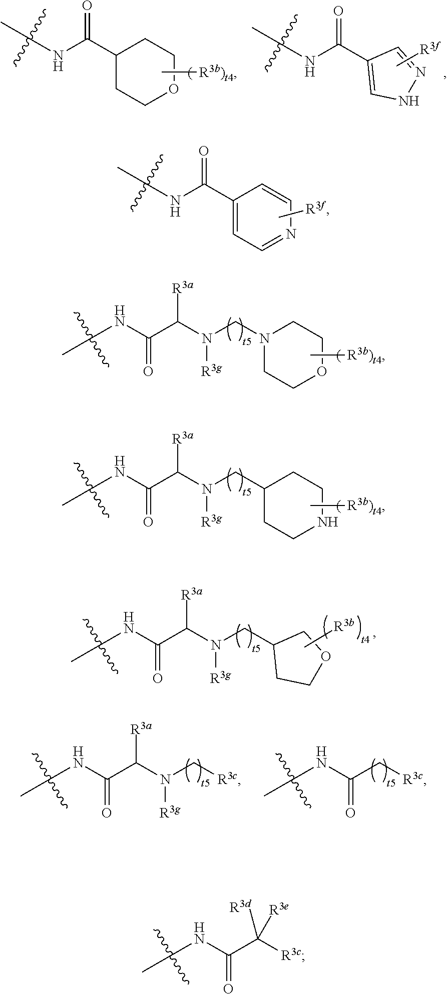
wherein, X⁶, X⁷, X⁸, and X⁹ are each independently selected from N, NH, O, S, CH or CH₂;
t4 and t5 are each independently selected from integers 0 to 6;
t6 and t7 are each independently selected from 0, 1 or 2;
R^{3a}, R^{3b}, R^{3c}, R^{3d}, R^{3e}, R^{3f} and R^{3g} are each independently selected from hydrogen, deuterium, halogen, cyano, oxo, hydroxyl, carboxyl, C₁₋₆ alkyl, C₁₋₆ alkoxy, C₃₋₆ cycloalkyl, 4- to 12-membered heterocycloalkyl, —(OCH₂CH₂)ₘOCH₃, NR^{X3a}R^{X3b}, —C(O)R^{X3c} wherein the said C₆ alkyl, C₁ alkoxy, C₃₆ cycloalkyl are optionally substituted with one or more substituents selected from halogen, hydroxyl, C₁₋₆ alkoxy;
or. R^{3d}, R^{3e} and the carbon atom to which they are attached together form C₃₋₆ cycloalkyl;
R^{X3a}, R^{X3b}, and R^{X3c} are each independently selected from hydrogen, C₁₋₆ alkyl, C₁₋₆ alkoxy or C₃₋₆ cycloalkyl;
m is an integer selected from 1 to 4; preferably 2 or 3;
preferably, R^{3a} is selected from hydrogen, deuterium, oxo, or C₁₋₆ alkyl; preferably from hydrogen, oxo, or methyl;
preferably, R^{3b} is selected from hydrogen, deuterium, halogen, cyano, oxo, hydroxyl, carboxyl, C₁₋₆ alkyl, C₁₋₆ alkoxy, —C₁₋₆ alkyl-OH, —C(O)C₁₋₆ alkyl, —C(O)C₃₋₆ cycloalkyl, C₃₋₆ cycloalkyl, or 4- to 12-membered heterocycloalkyl; wherein the said 4- to 12-membered heterocycloalkyl optionally contains 1, 2, or 3 heteroatoms selected from N and O as ring atoms;
preferably, R^{3b} is selected from hydrogen, deuterium, halogen, cyano, oxo, hydroxyl, carboxyl, methyl, methoxyl, —CH₂OH, —C(O)CH₃, —C(O)cyclopropyl, cyclopropyl, or morpholinyl;
preferably, R^{3c} is selected from hydrogen, deuterium, halogen, cyano, oxo, hydroxyl, carboxyl, amino, —(OCH₂CH₂)ₘOCH₃, —NR^{X3a}R^{X3b}, C₁₋₆ alkyl, C₁₋₆ alkoxy, halogen-substituted C₁₋₆ alkyl, C₃₋₆ cycloalkyl and halogen-substituted C₃₋₆ cycloalkyl, wherein the said C₁₋₆ alkyl or C₁₋₆ alkoxy is optionally substituted with one or more substituents selected from hydroxyl and C₁₋₆ alkoxy;
preferably, R^{3c} is selected from hydrogen, deuterium, halogen, cyano, oxo, hydroxyl, carboxyl, amino, dimethylamino, diethylamino, trifluoromethyl, methoxy, ethoxy, isopropyl, cyclopropyl, halogen-substituted cyclopropyl, —CH(OCH₃)₂, —OCH₂CH₂OH, —(OCH₂CH₂)₂OCH₃ or —(OCH₂CH₂)₃OCH₃;
Alternatively, R^{3d} and R^{3e} together with the carbon atom to which they are attached form C₃₋₆ cycloalkyl; preferably, R^{3d} and R^{3e} together with the carbon atom to which they are attached form cyclopropyl;
preferably, R^{3f} is hydrogen.

5. The compound according to claim 1, wherein the compound of formula (I) is a compound represented by formula (II) or formula (III): 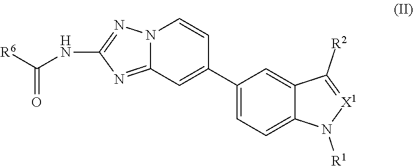
wherein X¹, R¹ and R² are each as defined for the compound of formula (I);
R⁶ is selected from hydrogen, deuterium, C₁₋₆ alkyl, C₁₋₆ alkoxy, C₃₋₆ cycloalkyl, 4- to 12-membered heterocycloalkyl, 5- or 6-membered heteroaryl, —C(O)NR^{5a}R^{5b}, —C(O)O—C₁₋₆ alkyl or —C(O)-(4- to 12-membered heterocycloalkyl), wherein the said C₁₋₆ alkyl, C₃₋₆ cycloalkyl, 4- to 12-membered heterocycloalkyl and 5- or 6-membered heteroaryl are optionally substituted with one or more substituents selected from halogen, oxo, hydroxyl, carboxyl, cyano, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl or 4- to 12-membered heterocycloalkyl, wherein two hydrogen atoms on any one carbon atom of the said C₁₋₆ alkyl are substituted to form C₃₋₆ cycloalkyl;
R^{5a} and R^{5b} are each independently selected from hydrogen, C₁₋₆ alkyl or C₁₋₆ alkoxy, wherein the said C₁₋₆ alkyl is optionally substituted with one or more halogens or C₃₋₆ cycloalkyl; 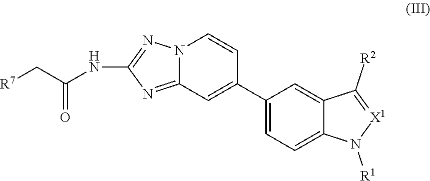
wherein X¹, R¹ and R² are each as defined for the compound of formula (I);
R⁷ is selected from —NR^{5a}R^{5b}, (OCH₂CH₂)ₘOCH₃, —NH—C₁₋₆ alkyl-O—C₁₋₆ alkyl, -4- to 12-membered heterocycloalkyl, —NH-4- to 12-membered heterocycloalkyl, —NH—C₁₋₆ alkyl-4- to 12-membered heterocycloalkyl or 5- or 6-membered heteroaryl, wherein the said C₁₋₆ alkyl, C₃₋₆ cycloalkyl, 4- to 12-membered heterocycloalkyl and 5- or 6-membered heteroaryl are optionally substituted with one or more substituents selected from halogen, oxo, hydroxyl, carboxyl, cyano, C₁₋₆ alkyl, —C₁₋₆ alkyl-OH, C₁₋₆ alkoxy, C₃₋₆ cycloalkyl, —C(O)—C₁₋₆ alkyl, —C(O)O—C₁₋₆ alkyl, —C(O)—C₃₋₆ cycloalkyl or 4- to 12-membered heterocycloalkyl;
R^{5a} and R^{5b} are each independently selected from hydrogen, C₁₋₆ alkyl or C₁₋₆ alkoxy, wherein the said C₁₋₆ alkyl is optionally substituted with one or more halogens or C₃₋₆ cycloalkyl;
preferably, the compound of formula (III) is a compound represented by formula (IIIa) or formula (IIIb): 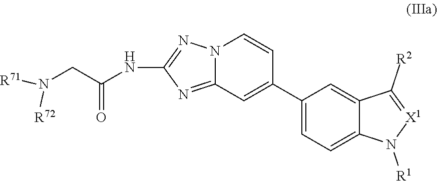
wherein X¹, R¹ and R² are each as defined for the compound of formula (I);
R⁷¹ and R⁷² are each independently selected from hydrogen, C₁₋₆ alkyl, C₁₋₆ alkoxy, 4- to 12-membered heterocycloalkyl and 5- or 6-membered heteroaryl, wherein the said C₁₋₆ alkyl is optionally substituted with one or more substituents selected from halogen, C₁₋₆ alkoxy, C₃₋₆ cycloalkyl, 4- to 12-membered heterocycloalkyl or —CH(OCH₃)₂; 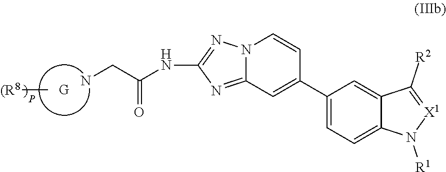
wherein X¹, R¹ and R² are each as defined for the compound of formula (I);
Ring G is selected from 5- or 6-membered nitrogen-containing heteroaryl or 5- to 12-membered nitrogen-containing heterocycloalkyl;
R⁸ is selected from hydrogen, halogen, cyano, hydroxyl, carboxyl, oxo, C₁₋₆ alkyl, C₁₋₆ alkoxy, —C₁₋₆ alkyl-OH, —C(O)—C₁₋₆ alkyl, —C(O)O—C₁₋₆ alkyl or —C(O)—C₃₋₆ cycloalkyl;
p is selected from 0, 1, 2 or 3;
preferably, R⁸ is selected from hydrogen, fluoro, cyano, hydroxyl, carboxyl, oxo, methyl, methoxy, hydroxymethyl, —C(O)CH₃, —C(O)-cyclopropyl or —C(O)OCH(CH₃)₃;
preferably, ring G is selected from

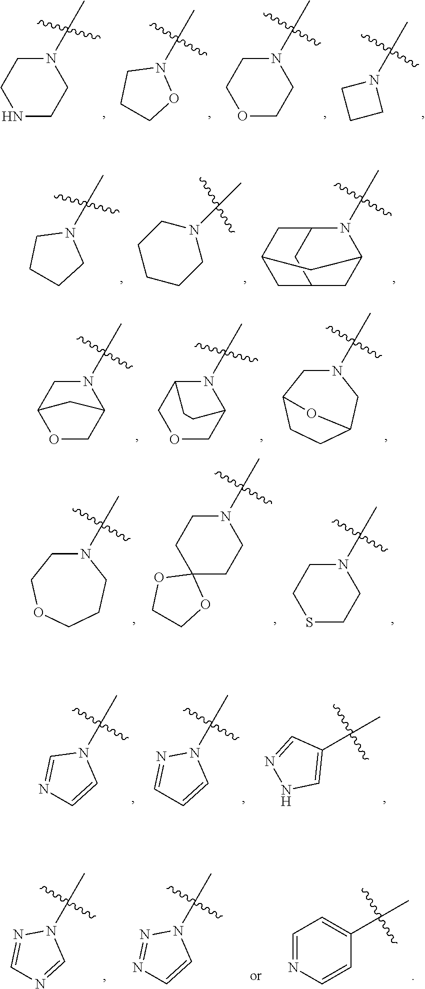

6. The compound according to any one of claims 1-5, wherein the said 4- to 12-membered heterocycloalkyl is selected from 4- to 7-membered monocyclic heterocycloalkyl, 6- to 8-membered bicyclic heterocycloalkyl, 6- to 10-membered bridged heterocycloalkyl, or 6- to 10-membered spiroheterocycloalkyl; the said 4- to 7-membered monocyclic heterocycloalkyl, 6- to 8-membered bicyclic heterocycloalkyl, 6- to 10-membered bridged heterocycloalkyl, or 6- to 10-membered spiroheterocycloalkyl preferably contains 1, 2, or 3 heteroatoms selected from N, S, or O as ring atoms;
preferably, the said 4- to 12-membered heterocycloalkyl is selected from pyrrolidinyl, piperidinyl, piperazinyl, oxetanyl, tetrahydrofuranyl, oxetanyl, isoxazolidinyl, morpholinyl, oxazepanyl, azetidinyl, pyrrolidinyl, piperidinyl, azaadamantyl, oxa-azabicycloheptyl, oxa-azabicyclooctyl, oxa-azaspirodecyl or thiomorpholinyl;
preferably, the said 4- to 12-membered heterocycloalkyl is selected from 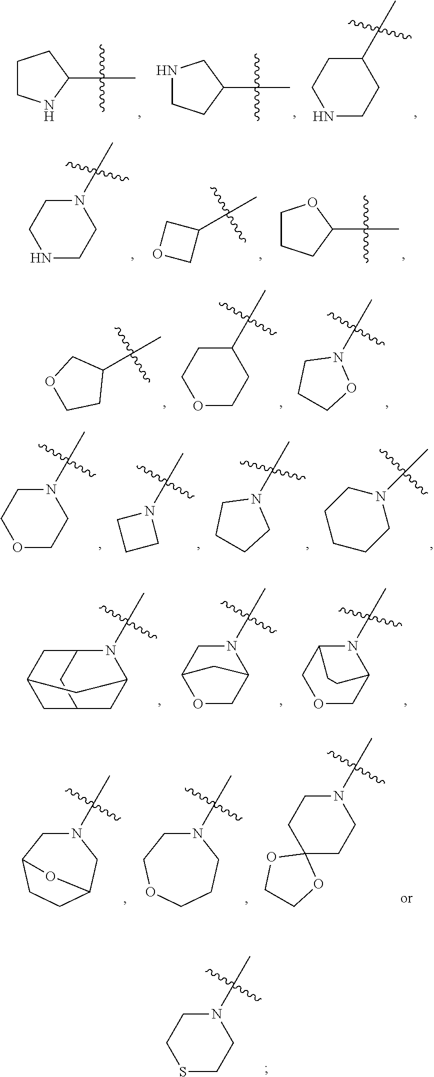
Alternatively, the said 5- or 6-membered heteroaryl is selected from 5- or 6-membered nitrogen-containing heteroaryl, wherein the said 5- or 6-membered nitrogen-containing heteroaryl optionally contains 1, 2 or 3 nitrogen atoms as ring atoms;
preferably, the said 5- or 6-membered heteroaryl is selected from imidazolyl, pyrazolyl, triazolyl or pyridyl;
preferably, the said 5- or 6-membered heteroaryl is selected from

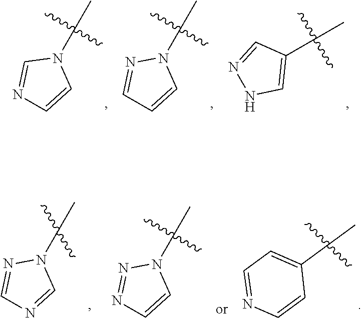 [SMILES: *n1ccnc1 ; *n1cccn1 ; *c1cn[nH]c1 ; *n1cncn1 ; *n1ccnn1 ; *c1ccncc1]

7. The following compounds, or pharmaceutically acceptable salts, solvates or stereoisomers thereof:

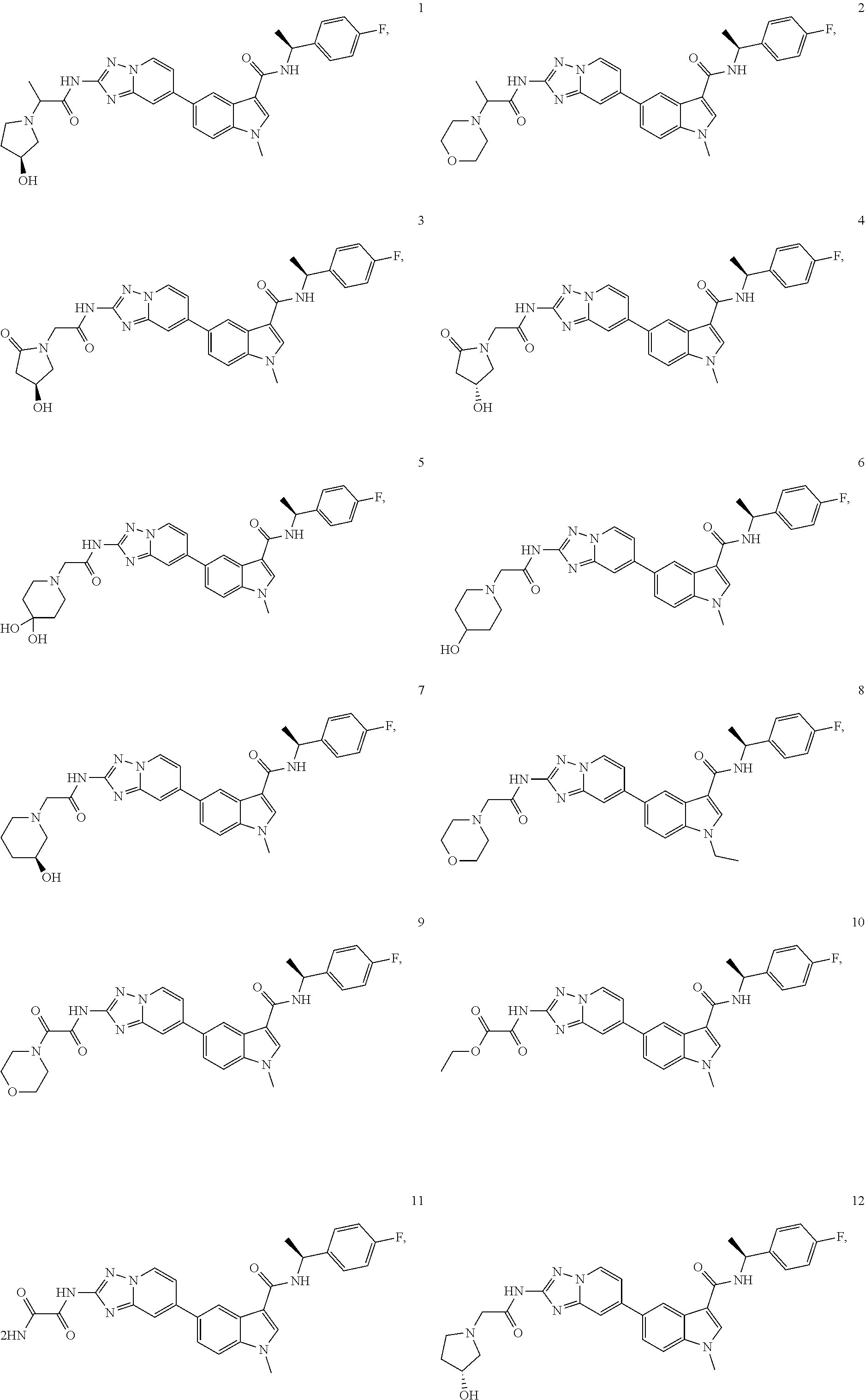

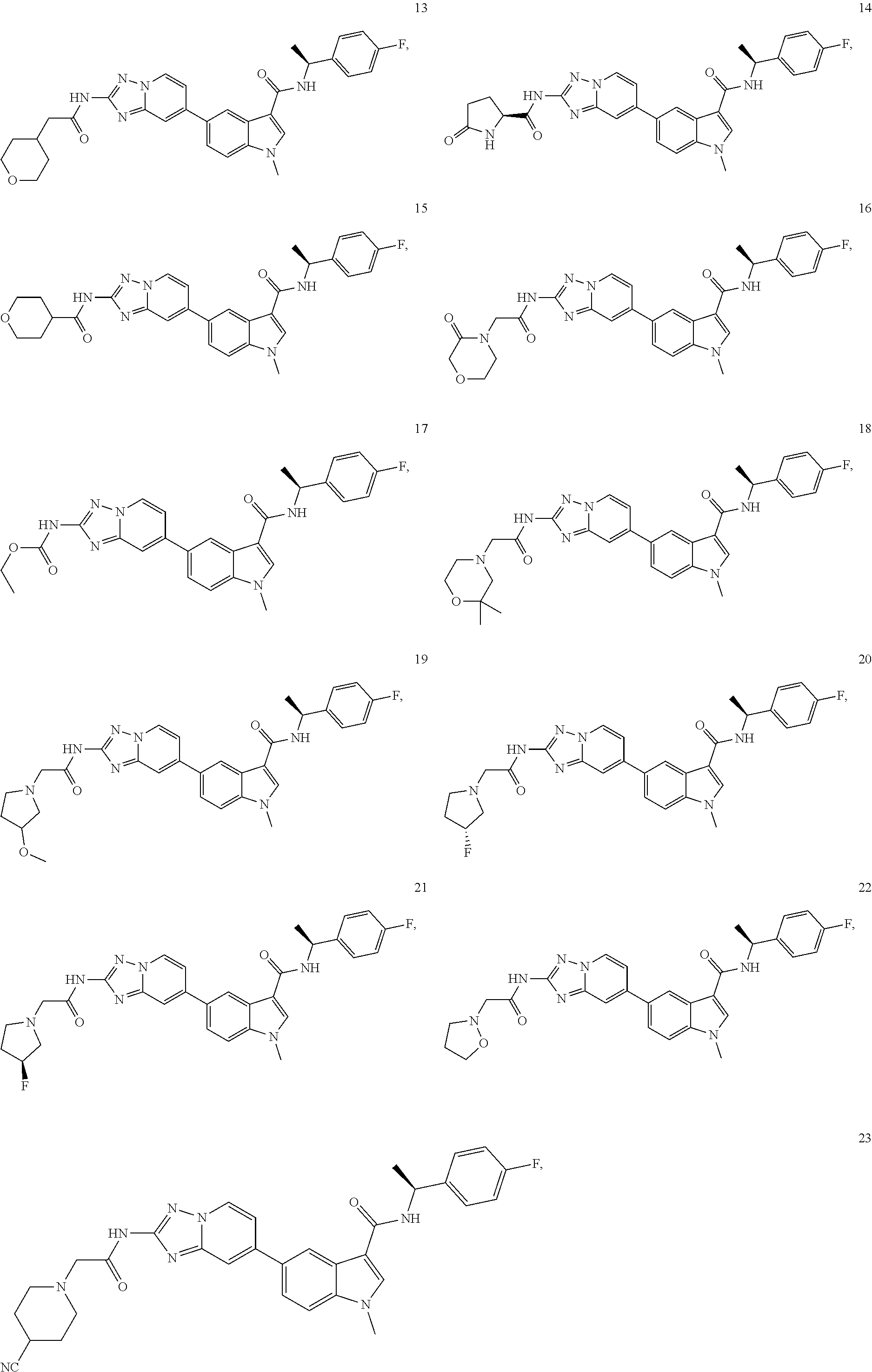

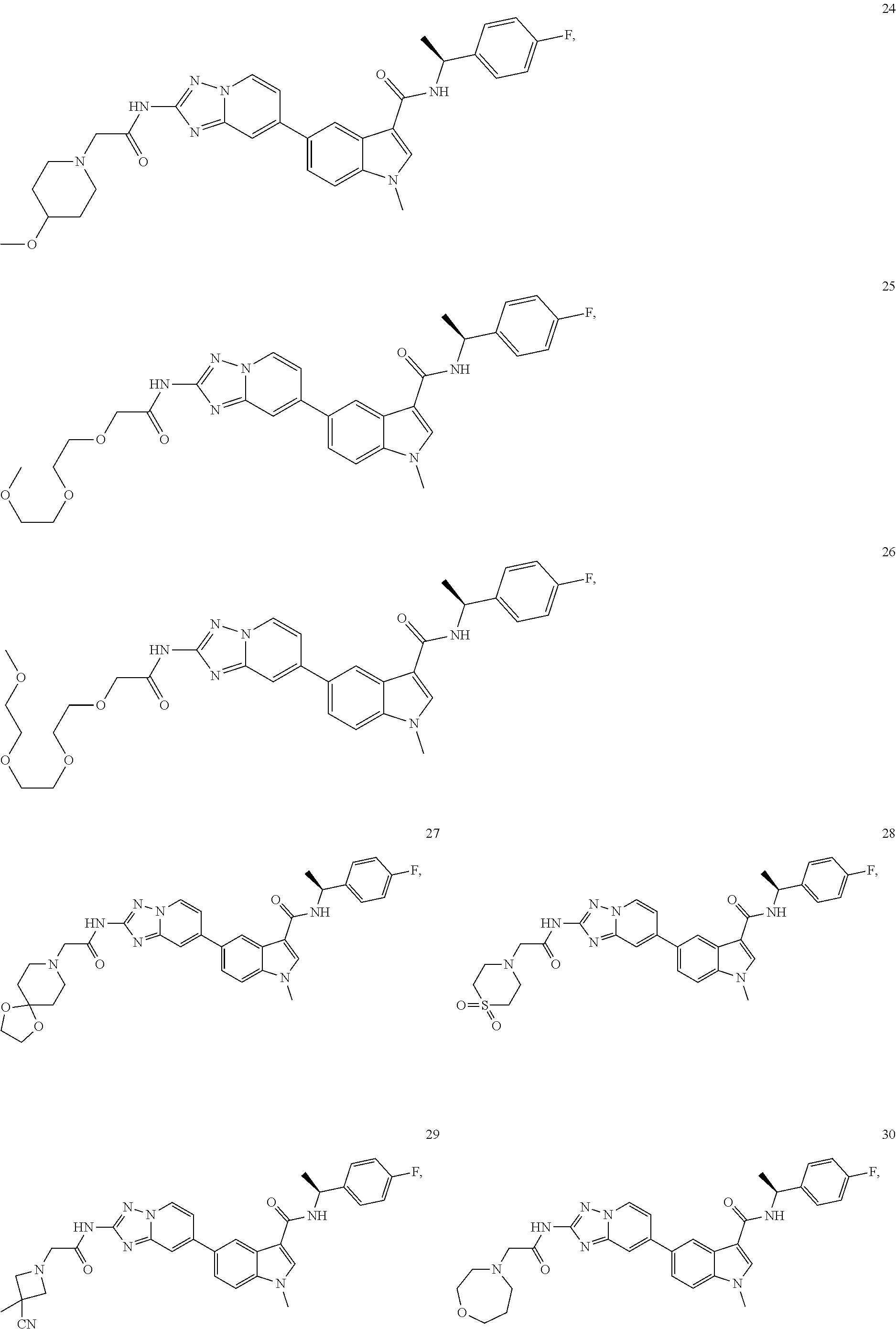

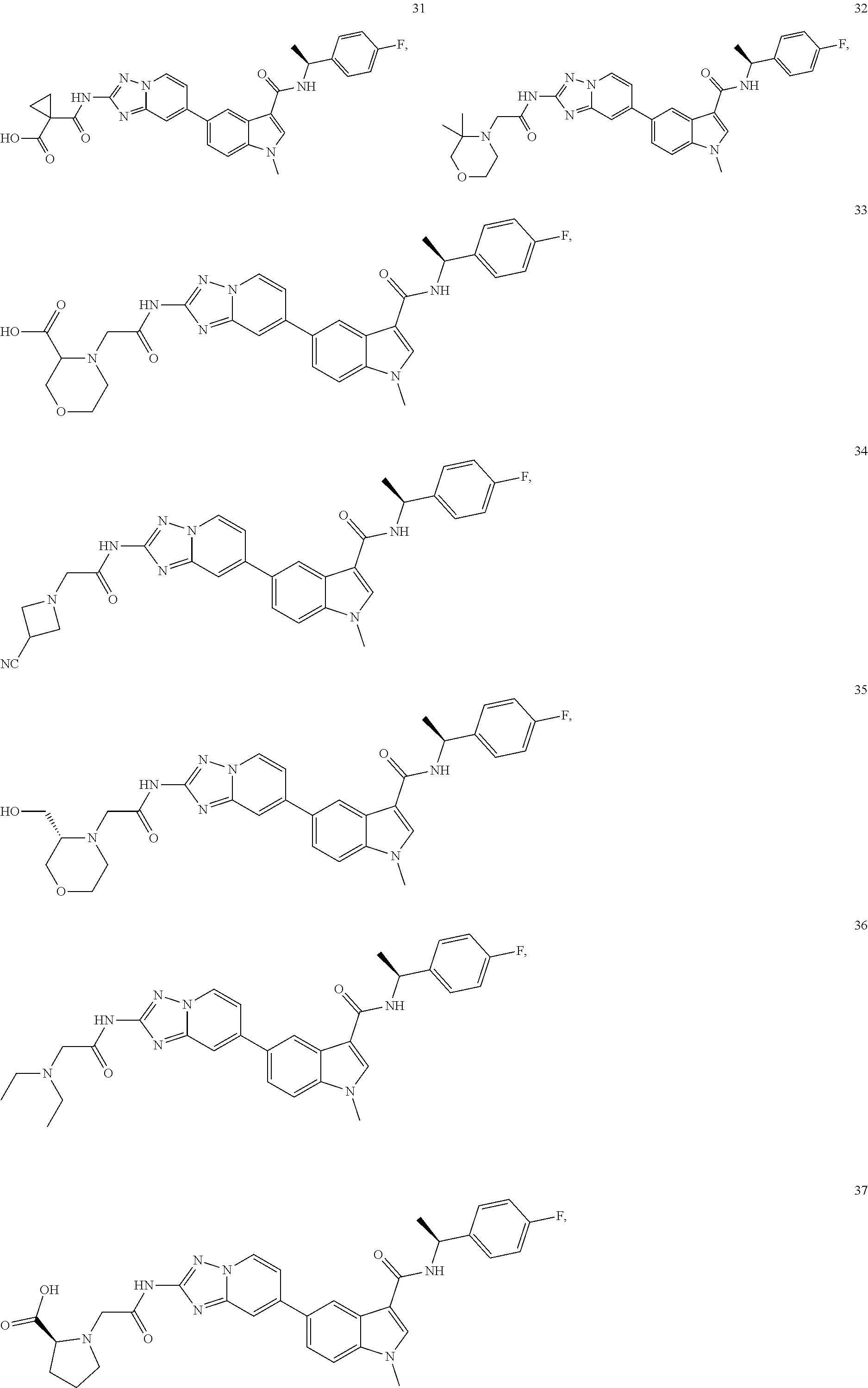

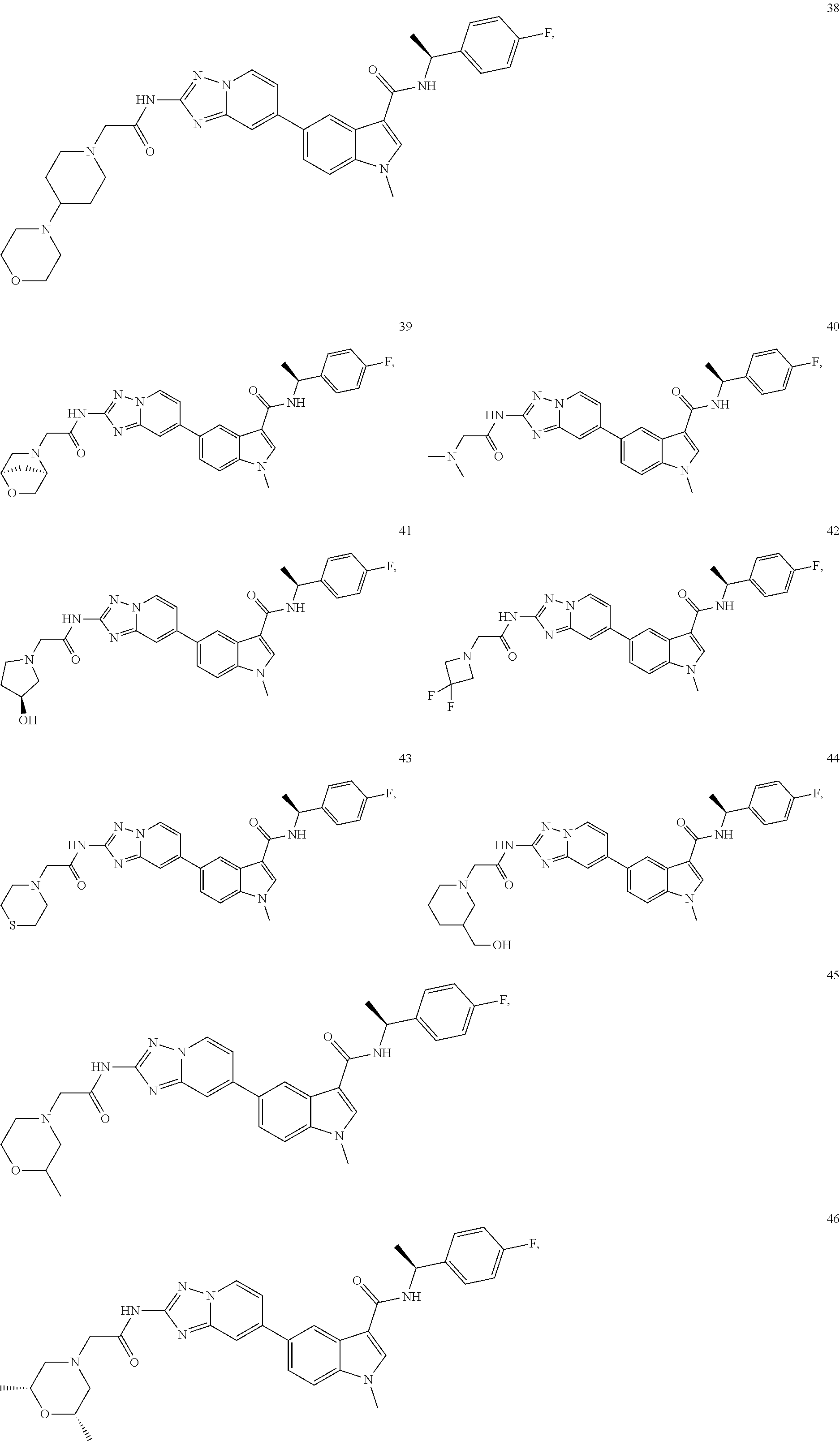

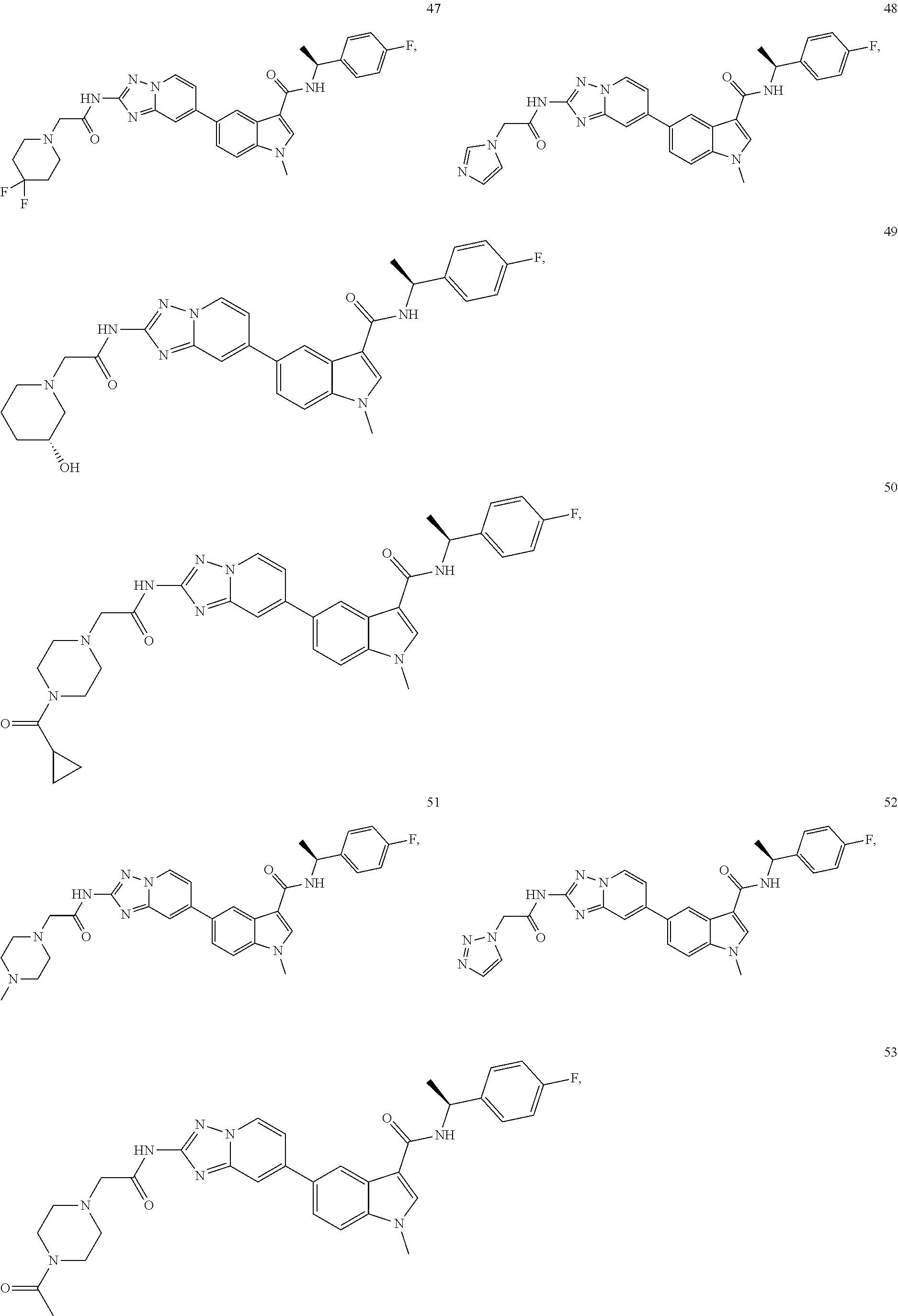

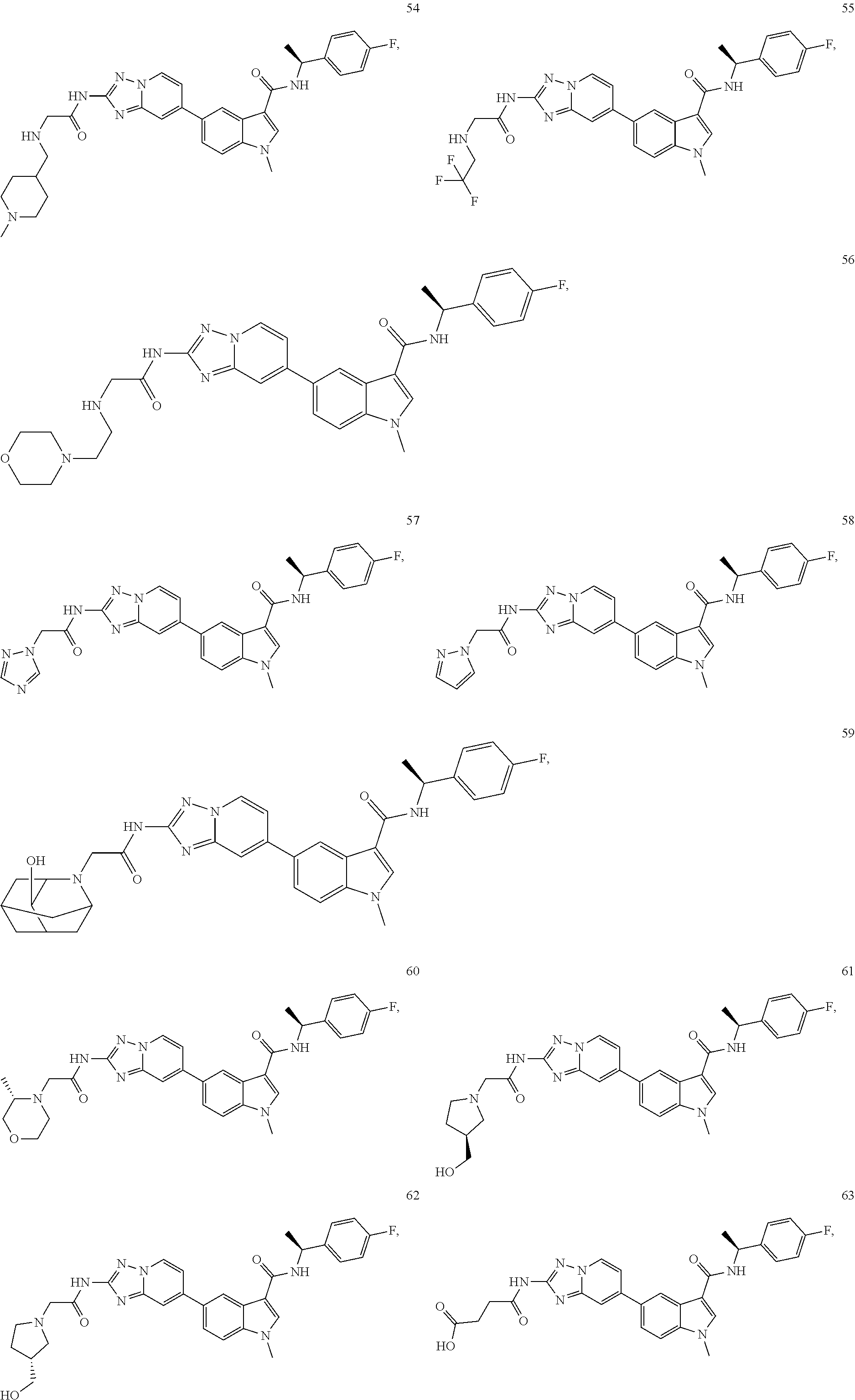

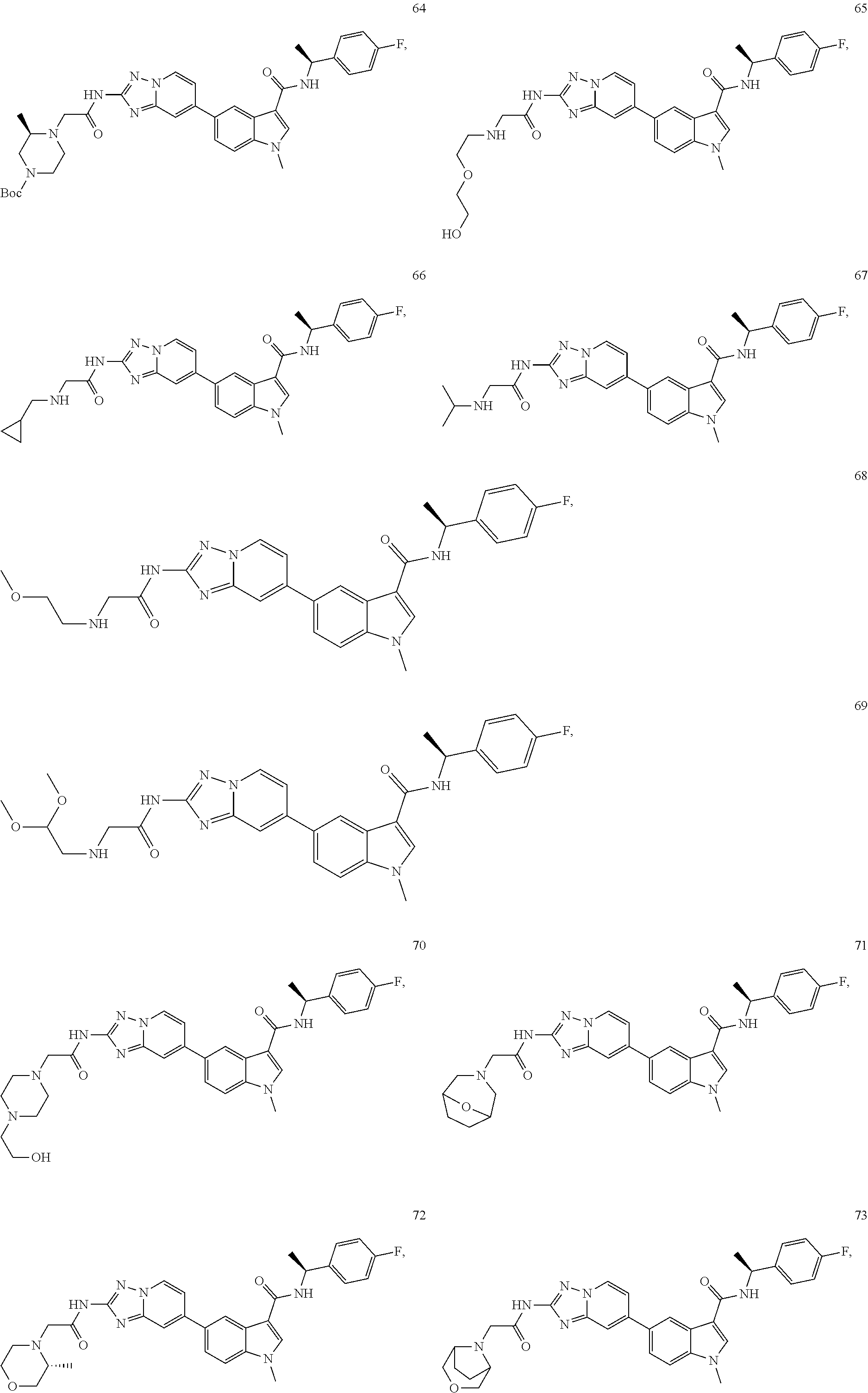

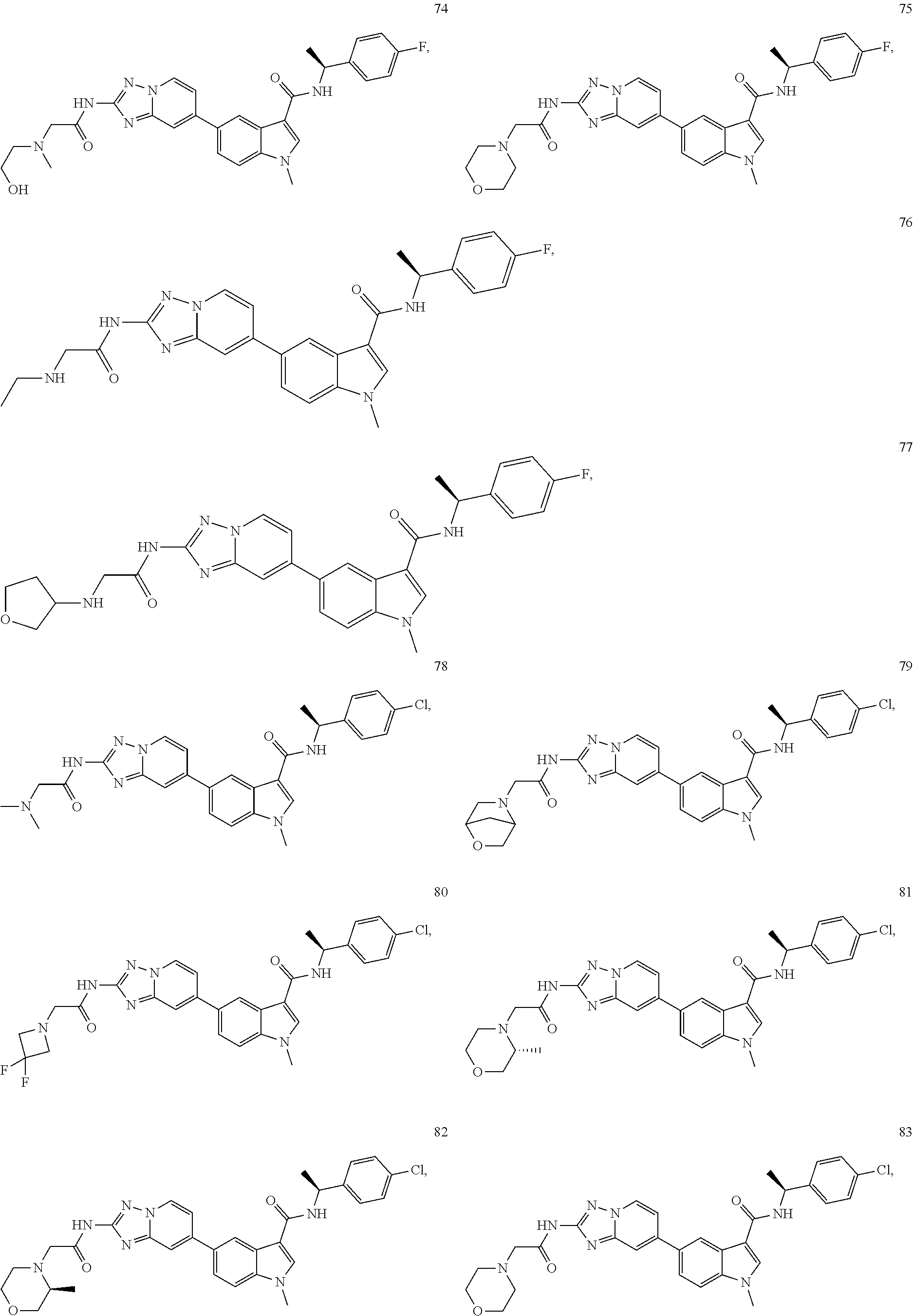

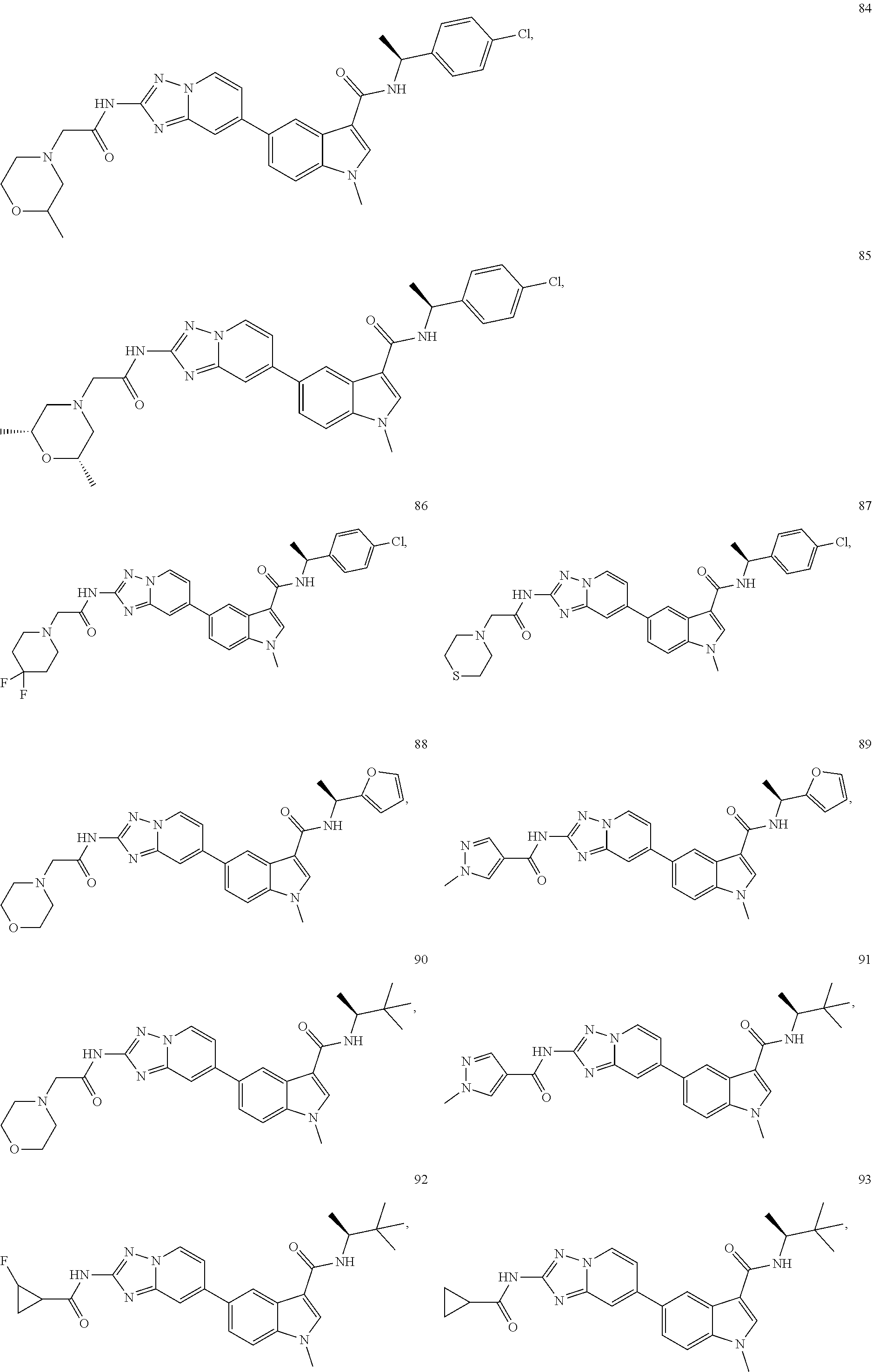

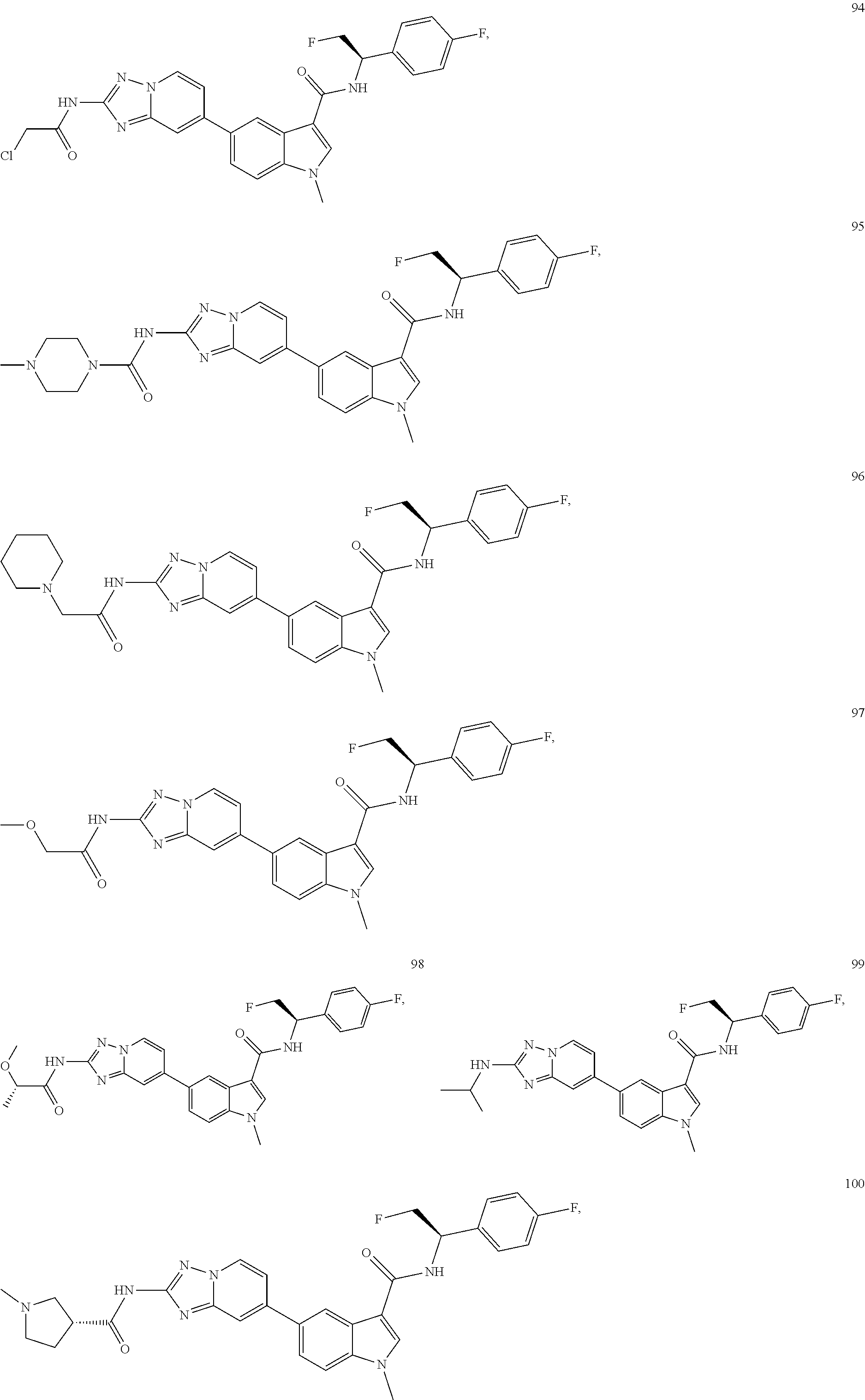

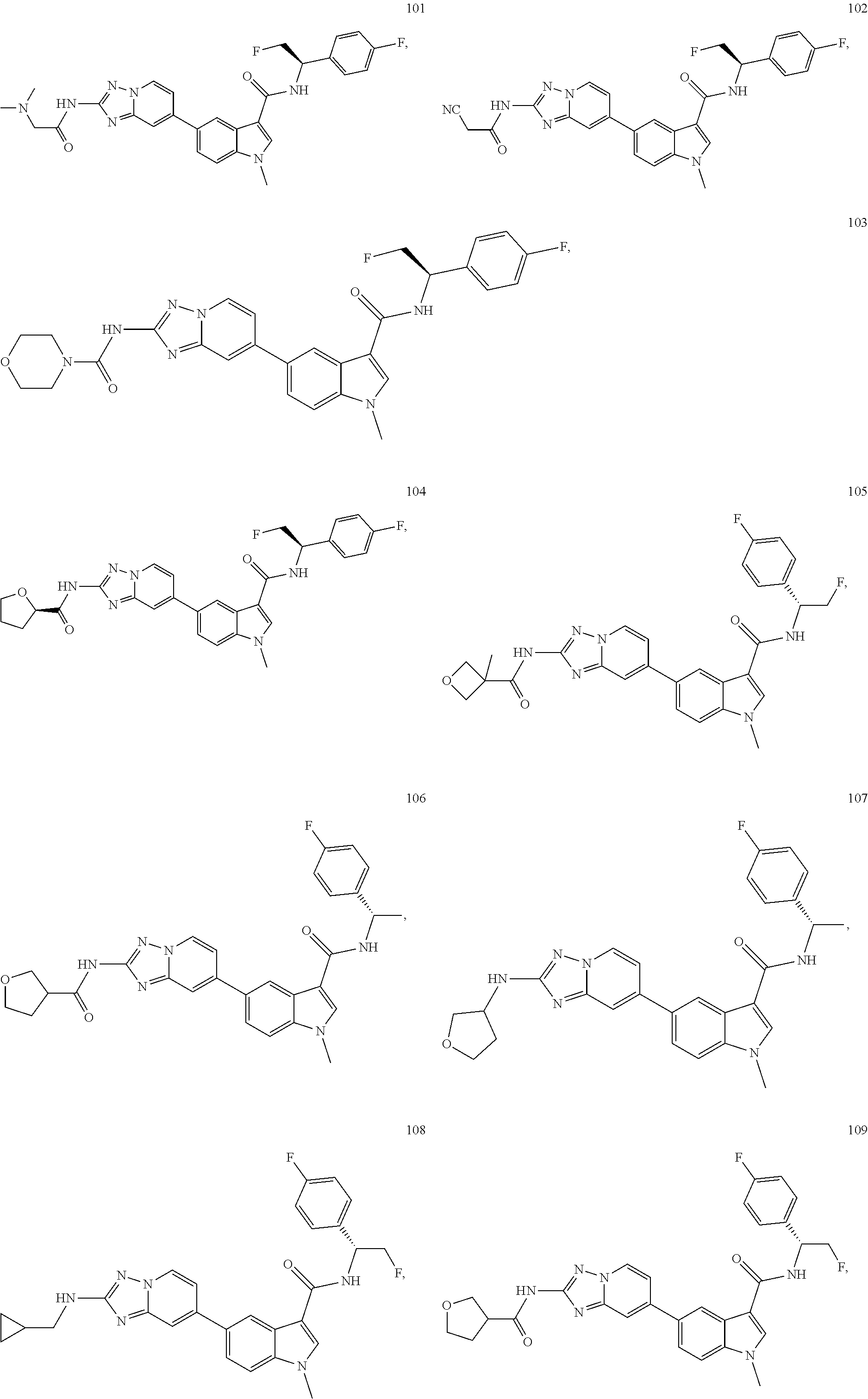

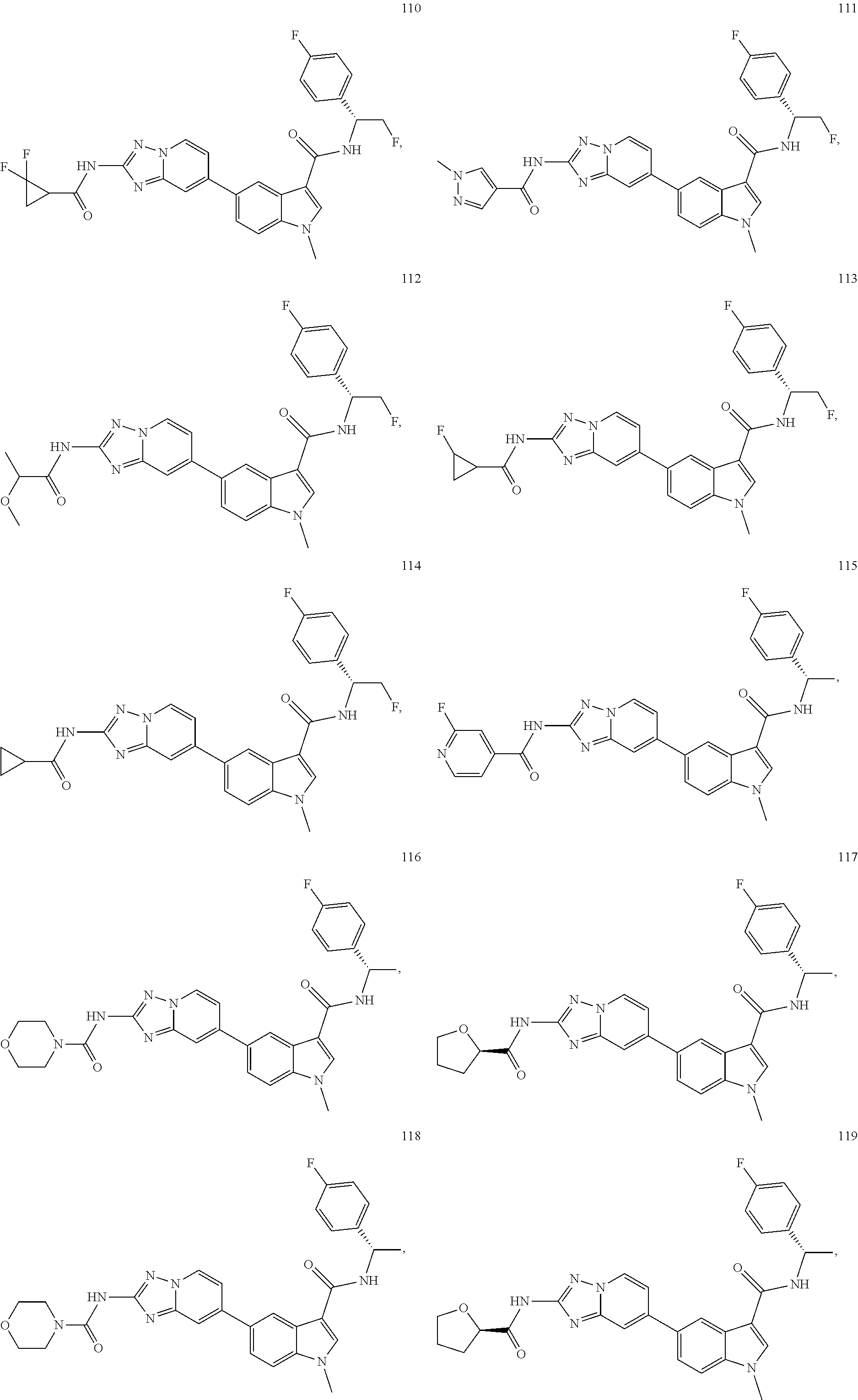

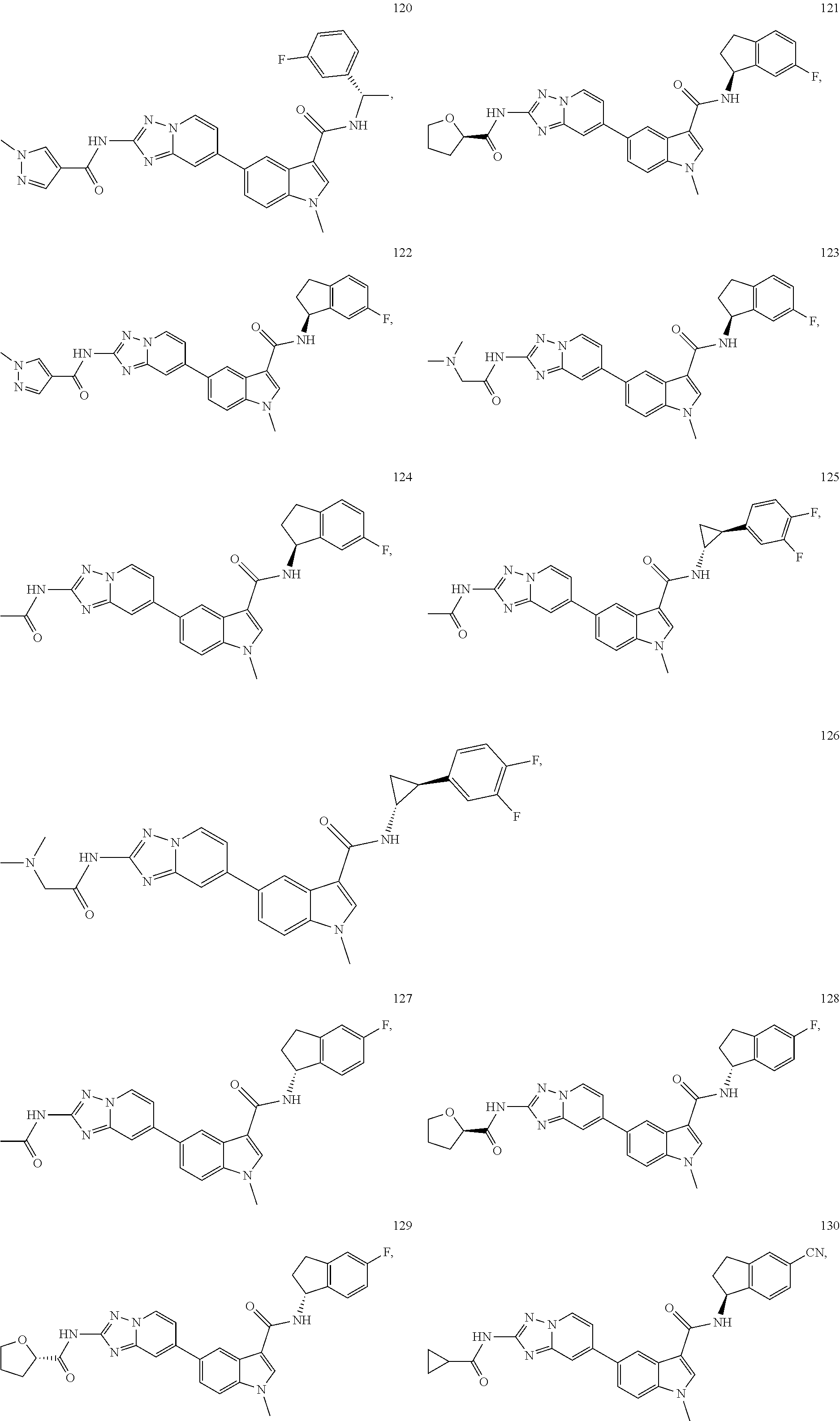

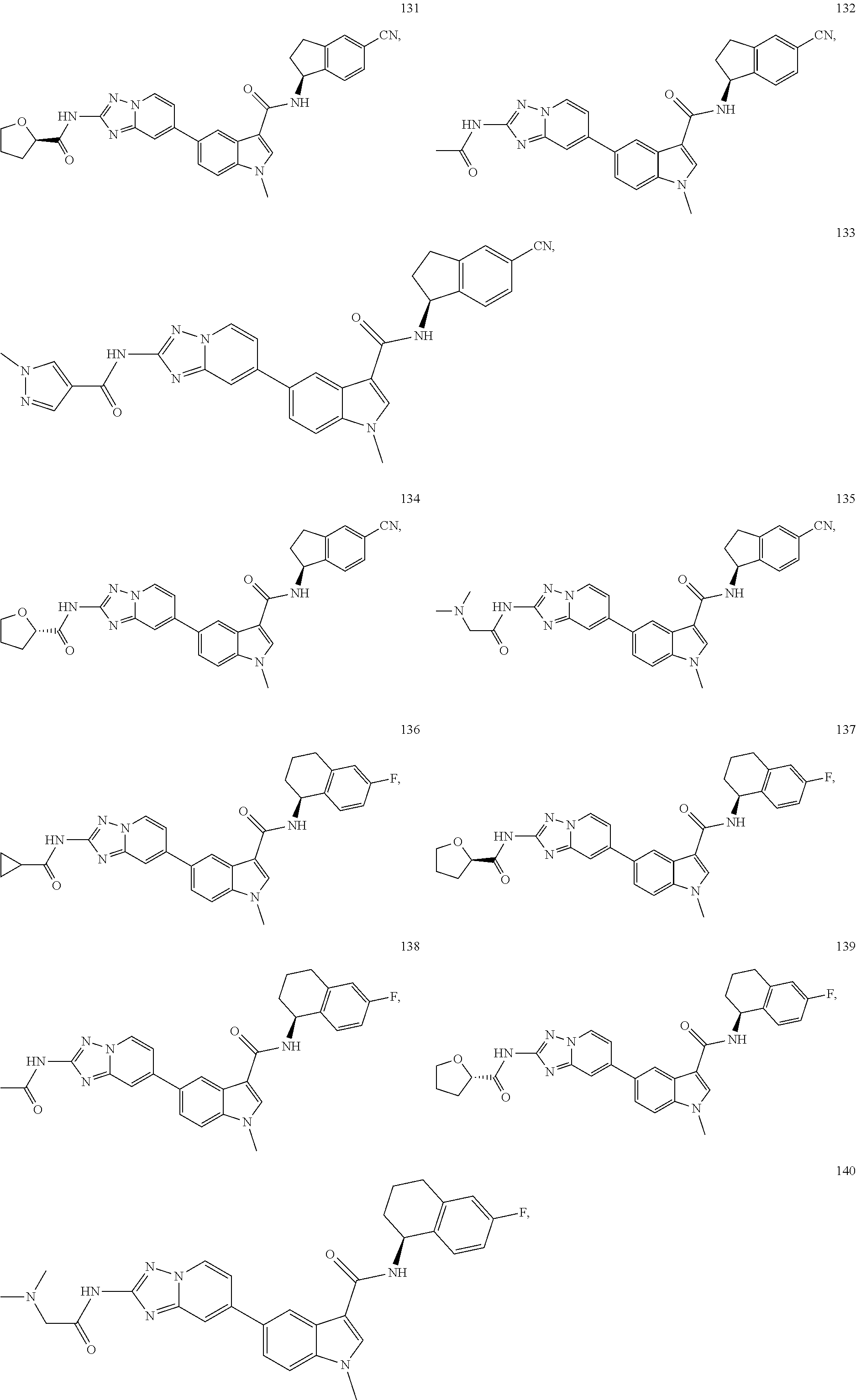

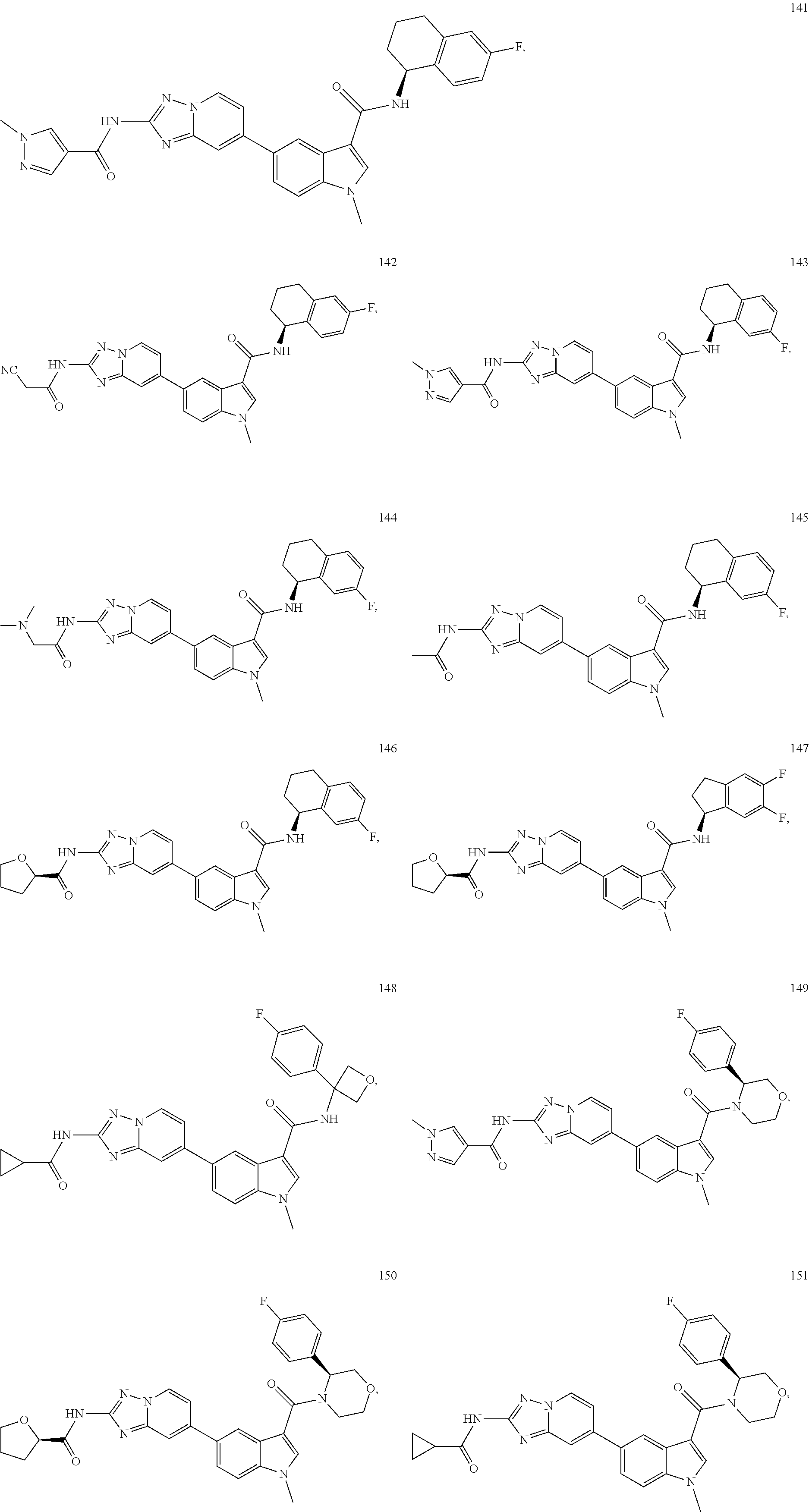

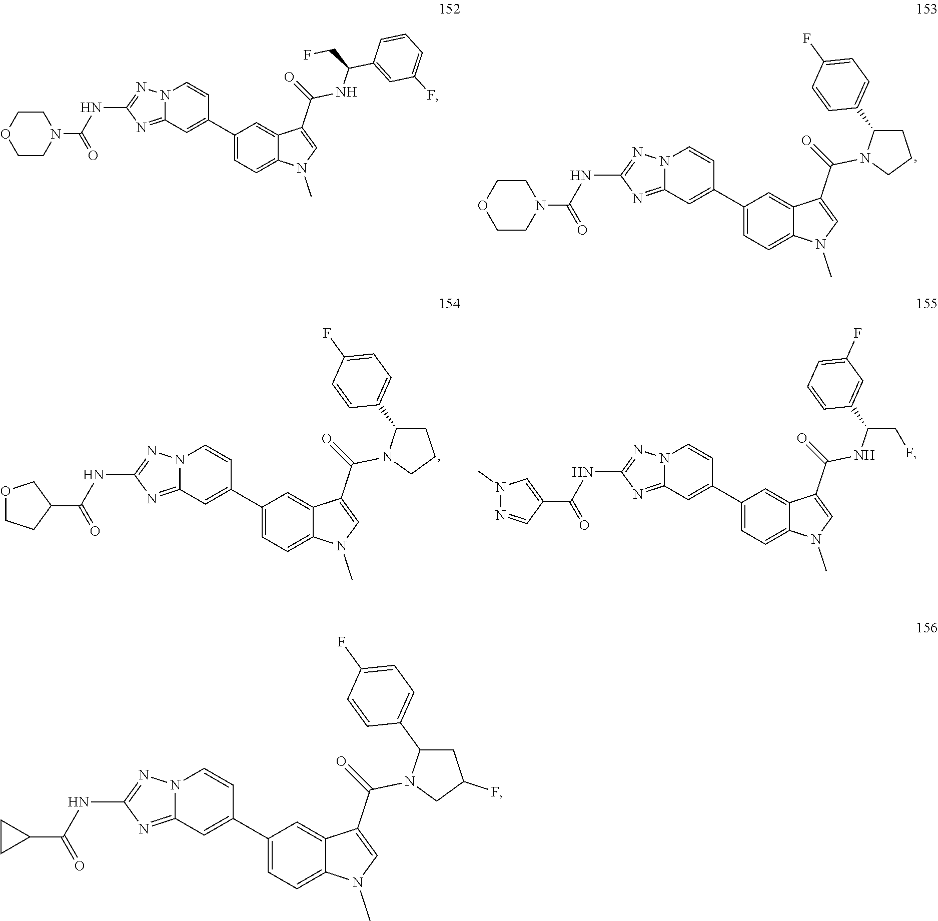 [SMILES: Cn1cc(C(=O)N[C@@H](CF)c2cccc(F)c2)c2cc(-c3ccn4nc(NC(=O)N5CCOCC5)nc4c3)ccc21 ; Cn1cc(C(=O)N2CCC[C@H]2c2ccc(F)cc2)c2cc(-c3ccn4nc(NC(=O)N5CCOCC5)nc4c3)ccc21 ; Cn1cc(C(=O)N2CCC[C@H]2c2ccc(F)cc2)c2cc(-c3ccn4nc(NC(=O)C5CCOC5)nc4c3)ccc21 ; Cn1cc(C(=O)Nc2nc3cc(-c4ccc5c(c4)c(C(=O)N[C@@H](CF)c4cccc(F)c4)cn5C)ccn3n2)cn1 ; Cn1cc(C(=O)N2CC(F)CC2c2ccc(F)cc2)c2cc(-c3ccn4nc(NC(=O)C5CC5)nc4c3)ccc21]

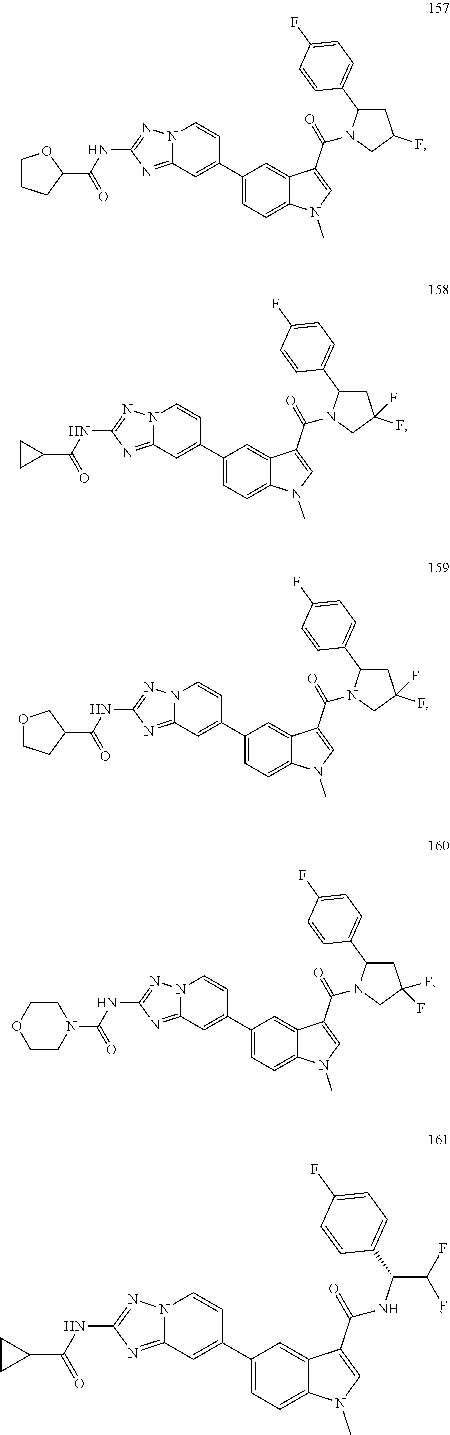 [SMILES: Cn1cc(C(=O)N2CC(F)CC2c2ccc(F)cc2)c2cc(-c3ccn4nc(NC(=O)C5CCCO5)nc4c3)ccc21 ; Cn1cc(C(=O)N2CC(F)(F)CC2c2ccc(F)cc2)c2cc(-c3ccn4nc(NC(=O)C5CC5)nc4c3)ccc21 ; Cn1cc(C(=O)N2CC(F)(F)CC2c2ccc(F)cc2)c2cc(-c3ccn4nc(NC(=O)C5CCOC5)nc4c3)ccc21 ; Cn1cc(C(=O)N2CC(F)(F)CC2c2ccc(F)cc2)c2cc(-c3ccn4nc(NC(=O)N5CCOCC5)nc4c3)ccc21 ; Cn1cc(C(=O)N[C@H](c2ccc(F)cc2)C(F)F)c2cc(-c3ccn4nc(NC(=O)C5CC5)nc4c3)ccc21]

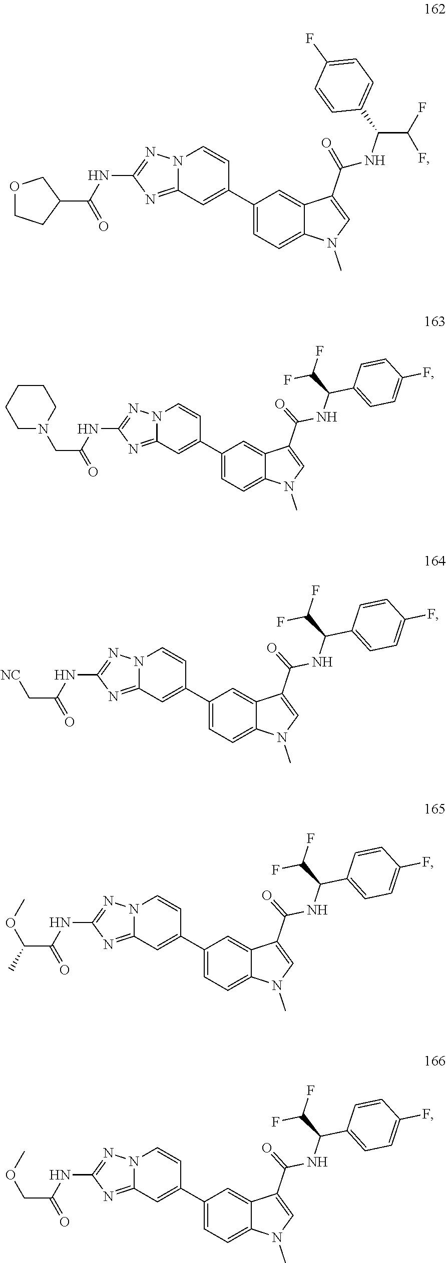 [SMILES: Cn1cc(C(=O)N[C@H](c2ccc(F)cc2)C(F)F)c2cc(-c3ccn4nc(NC(=O)C5CCOC5)nc4c3)ccc21 ; Cn1cc(C(=O)N[C@H](c2ccc(F)cc2)C(F)F)c2cc(-c3ccn4nc(NC(=O)CN5CCCCC5)nc4c3)ccc21 ; Cn1cc(C(=O)N[C@H](c2ccc(F)cc2)C(F)F)c2cc(-c3ccn4nc(NC(=O)CC#N)nc4c3)ccc21 ; CO[C@@H](C)C(=O)Nc1nc2cc(-c3ccc4c(c3)c(C(=O)N[C@H](c3ccc(F)cc3)C(F)F)cn4C)ccn2n1 ; COCC(=O)Nc1nc2cc(-c3ccc4c(c3)c(C(=O)N[C@H](c3ccc(F)cc3)C(F)F)cn4C)ccn2n1]

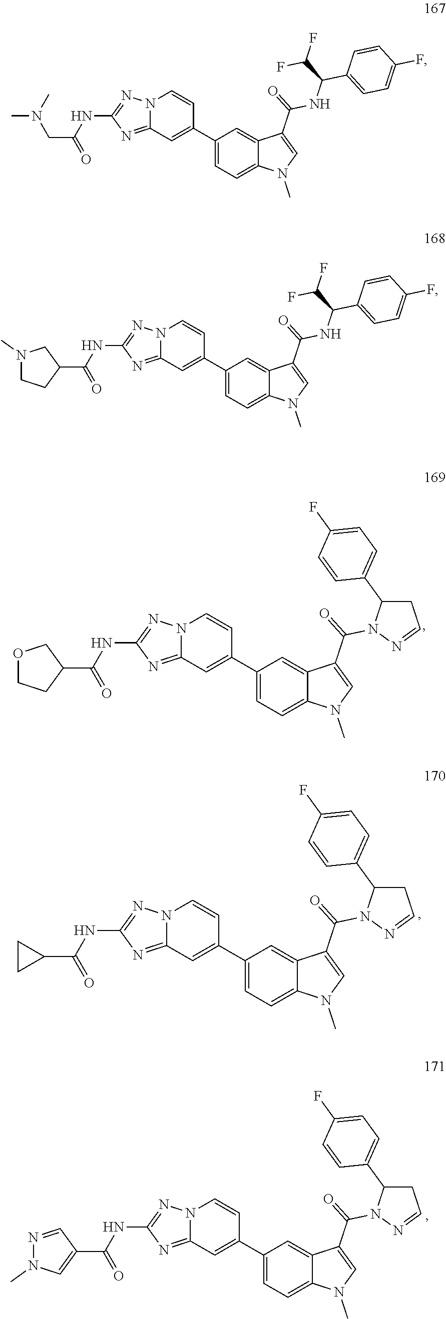 [SMILES: CN(C)CC(=O)Nc1nc2cc(-c3ccc4c(c3)c(C(=O)N[C@H](c3ccc(F)cc3)C(F)F)cn4C)ccn2n1 ; CN1CCC(C(=O)Nc2nc3cc(-c4ccc5c(c4)c(C(=O)N[C@H](c4ccc(F)cc4)C(F)F)cn5C)ccn3n2)C1 ; Cn1cc(C(=O)N2N=CCC2c2ccc(F)cc2)c2cc(-c3ccn4nc(NC(=O)C5CCOC5)nc4c3)ccc21 ; Cn1cc(C(=O)N2N=CCC2c2ccc(F)cc2)c2cc(-c3ccn4nc(NC(=O)C5CC5)nc4c3)ccc21 ; Cn1cc(C(=O)Nc2nc3cc(-c4ccc5c(c4)c(C(=O)N4N=CCC4c4ccc(F)cc4)cn5C)ccn3n2)cn1]

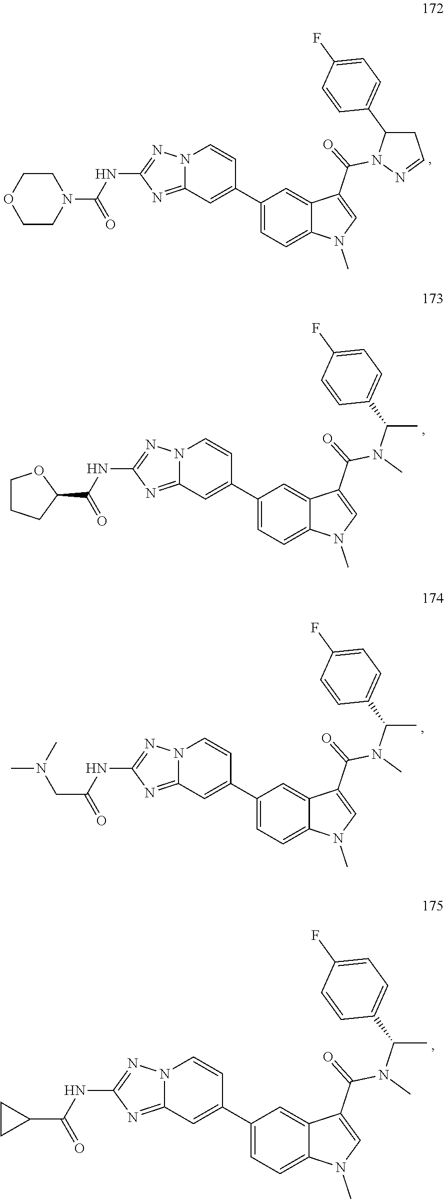

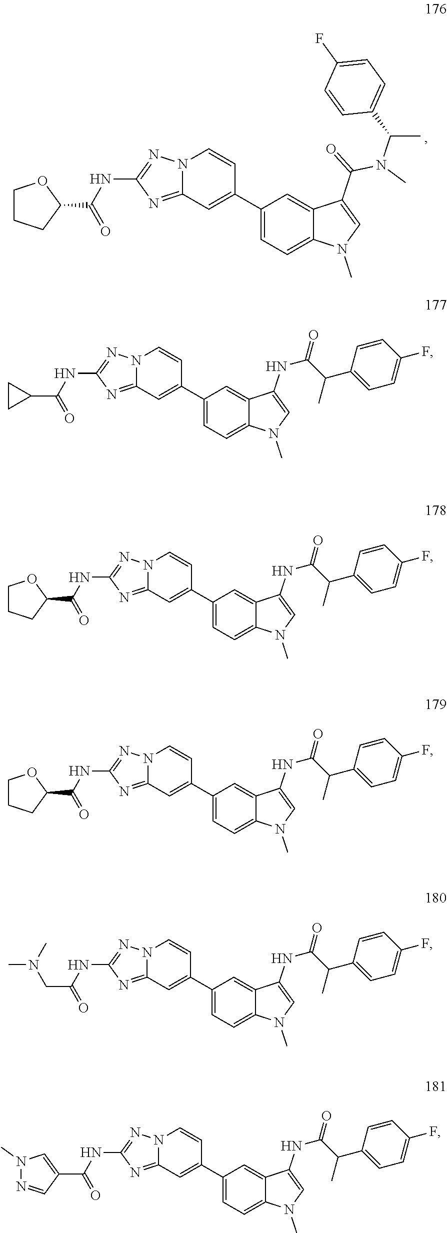

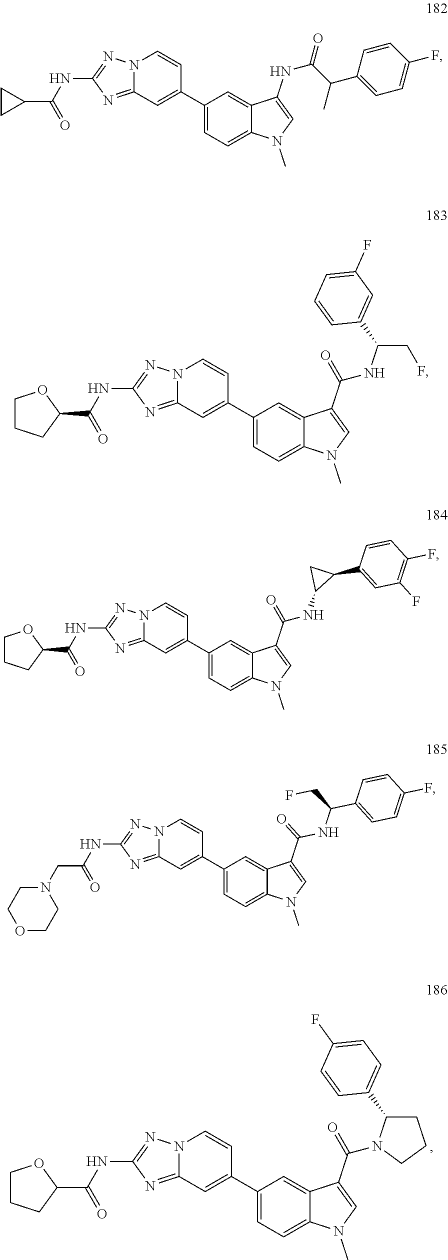 [SMILES: CC(C(=O)Nc1cn(C)c2ccc(-c3ccn4nc(NC(=O)C5CC5)nc4c3)cc12)c1ccc(F)cc1 ; Cn1cc(C(=O)N[C@@H](CF)c2cccc(F)c2)c2cc(-c3ccn4nc(NC(=O)[C@H]5CCCO5)nc4c3)ccc21 ; Cn1cc(C(=O)N[C@@H]2C[C@H]2c2ccc(F)c(F)c2)c2cc(-c3ccn4nc(NC(=O)[C@H]5CCCO5)nc4c3)ccc21 ; Cn1cc(C(=O)N[C@@H](CF)c2ccc(F)cc2)c2cc(-c3ccn4nc(NC(=O)CN5CCOCC5)nc4c3)ccc21 ; Cn1cc(C(=O)N2CCC[C@H]2c2ccc(F)cc2)c2cc(-c3ccn4nc(NC(=O)C5CCCO5)nc4c3)ccc21]

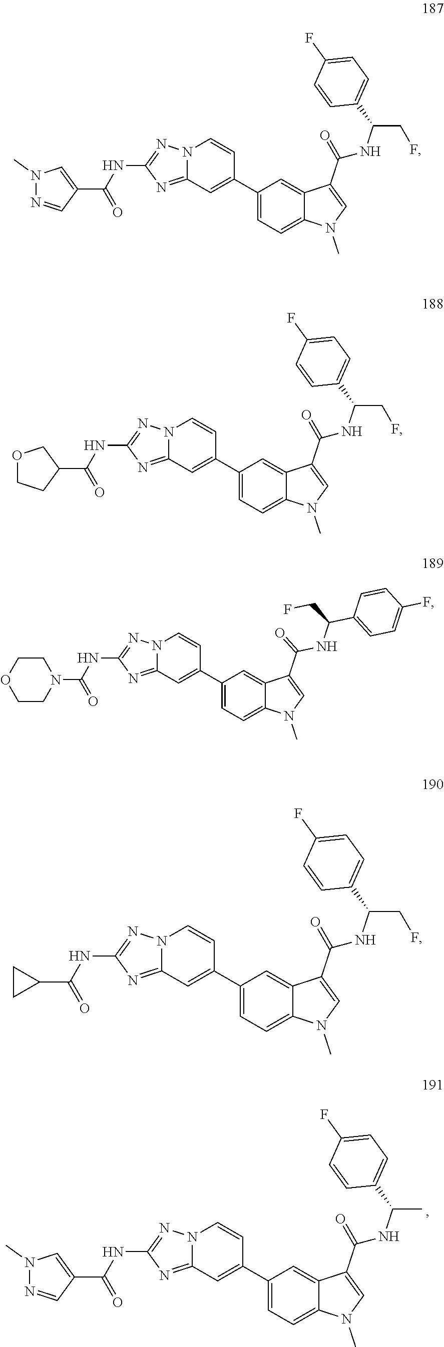 [SMILES: Cn1cc(C(=O)Nc2nc3cc(-c4ccc5c(c4)c(C(=O)N[C@@H](CF)c4ccc(F)cc4)cn5C)ccn3n2)cn1 ; Cn1cc(C(=O)N[C@@H](CF)c2ccc(F)cc2)c2cc(-c3ccn4nc(NC(=O)C5CCOC5)nc4c3)ccc21 ; Cn1cc(C(=O)N[C@@H](CF)c2ccc(F)cc2)c2cc(-c3ccn4nc(NC(=O)N5CCOCC5)nc4c3)ccc21 ; Cn1cc(C(=O)N[C@@H](CF)c2ccc(F)cc2)c2cc(-c3ccn4nc(NC(=O)C5CC5)nc4c3)ccc21 ; C[C@H](NC(=O)c1cn(C)c2ccc(-c3ccn4nc(NC(=O)c5cnn(C)c5)nc4c3)cc12)c1ccc(F)cc1]

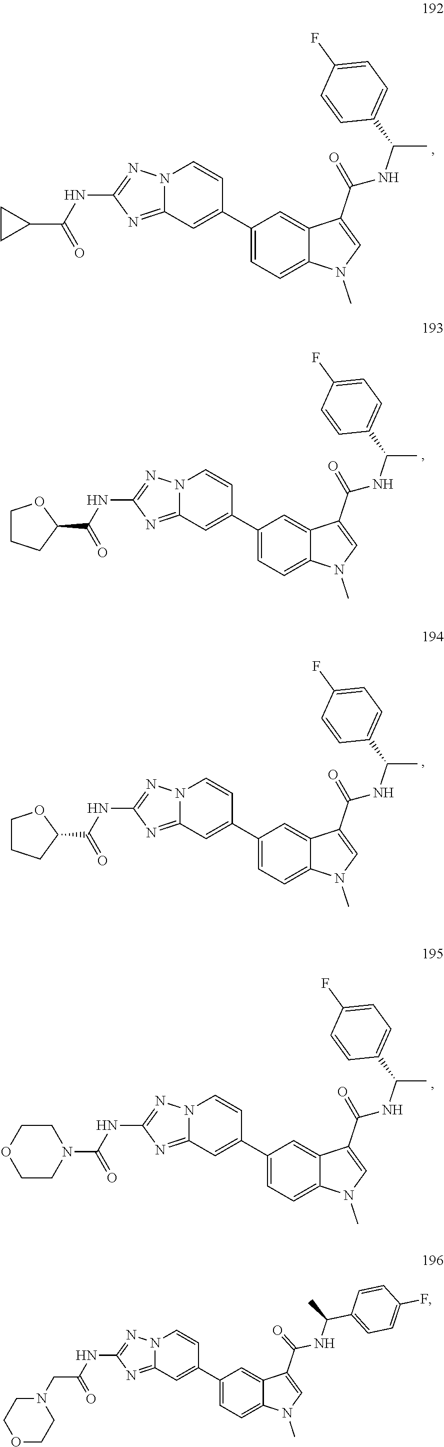 [SMILES: C[C@H](NC(=O)c1cn(C)c2ccc(-c3ccn4nc(NC(=O)C5CC5)nc4c3)cc12)c1ccc(F)cc1 ; C[C@H](NC(=O)c1cn(C)c2ccc(-c3ccn4nc(NC(=O)[C@H]5CCCO5)nc4c3)cc12)c1ccc(F)cc1 ; C[C@H](NC(=O)c1cn(C)c2ccc(-c3ccn4nc(NC(=O)[C@@H]5CCCO5)nc4c3)cc12)c1ccc(F)cc1 ; C[C@H](NC(=O)c1cn(C)c2ccc(-c3ccn4nc(NC(=O)N5CCOCC5)nc4c3)cc12)c1ccc(F)cc1 ; C[C@H](NC(=O)c1cn(C)c2ccc(-c3ccn4nc(NC(=O)CN5CCOCC5)nc4c3)cc12)c1ccc(F)cc1]

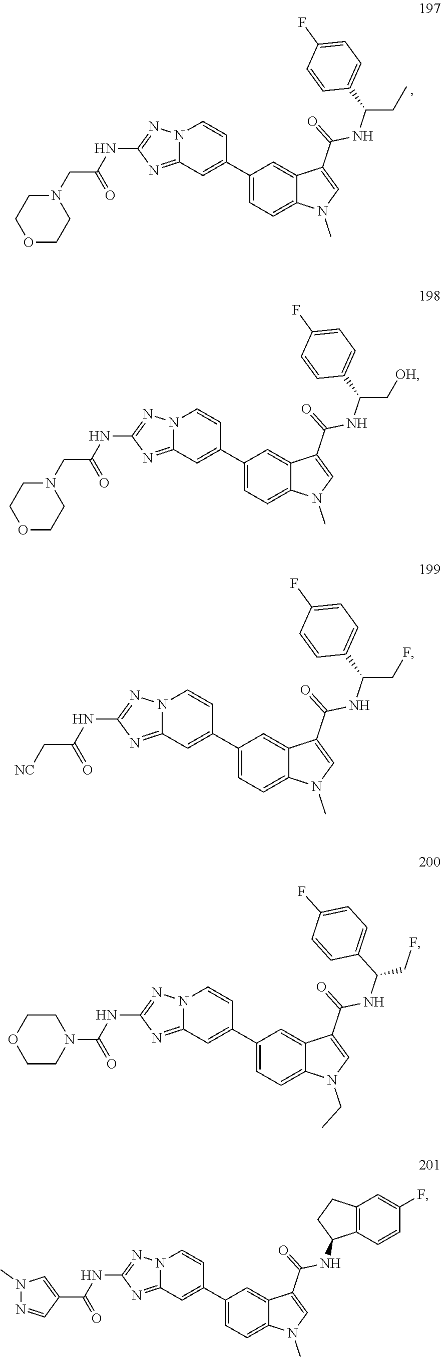

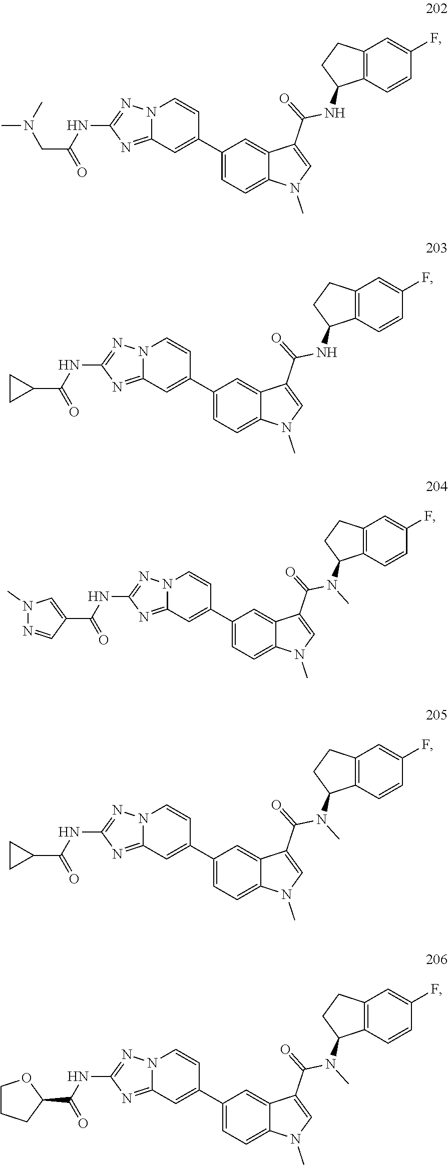 [SMILES: CN(C)CC(=O)Nc1nc2cc(-c3ccc4c(c3)c(C(=O)N[C@H]3CCc5cc(F)ccc53)cn4C)ccn2n1 ; Cn1cc(C(=O)N[C@H]2CCc3cc(F)ccc32)c2cc(-c3ccn4nc(NC(=O)C5CC5)nc4c3)ccc21 ; CN(C(=O)c1cn(C)c2ccc(-c3ccn4nc(NC(=O)c5cnn(C)c5)nc4c3)cc12)[C@H]1CCc2cc(F)ccc21 ; CN(C(=O)c1cn(C)c2ccc(-c3ccn4nc(NC(=O)C5CC5)nc4c3)cc12)[C@H]1CCc2cc(F)ccc21 ; CN(C(=O)c1cn(C)c2ccc(-c3ccn4nc(NC(=O)[C@H]5CCCO5)nc4c3)cc12)[C@H]1CCc2cc(F)ccc21]

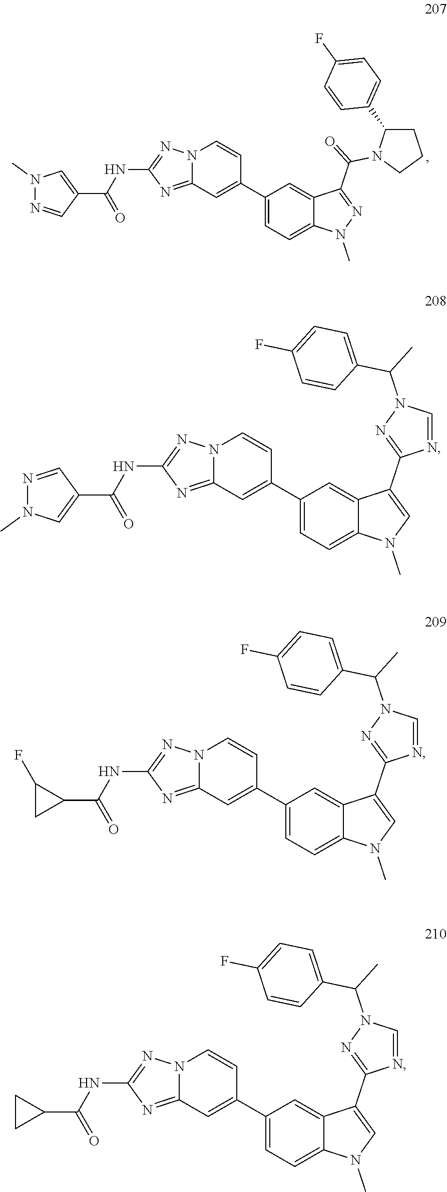 [SMILES: Cn1cc(C(=O)Nc2nc3cc(-c4ccc5c(c4)c(C(=O)N4CCC[C@H]4c4ccc(F)cc4)nn5C)ccn3n2)cn1 ; CC(c1ccc(F)cc1)n1cnc(-c2cn(C)c3ccc(-c4ccn5nc(NC(=O)c6cnn(C)c6)nc5c4)cc23)n1 ; CC(c1ccc(F)cc1)n1cnc(-c2cn(C)c3ccc(-c4ccn5nc(NC(=O)C6CC6F)nc5c4)cc23)n1 ; CC(c1ccc(F)cc1)n1cnc(-c2cn(C)c3ccc(-c4ccn5nc(NC(=O)C6CC6)nc5c4)cc23)n1]

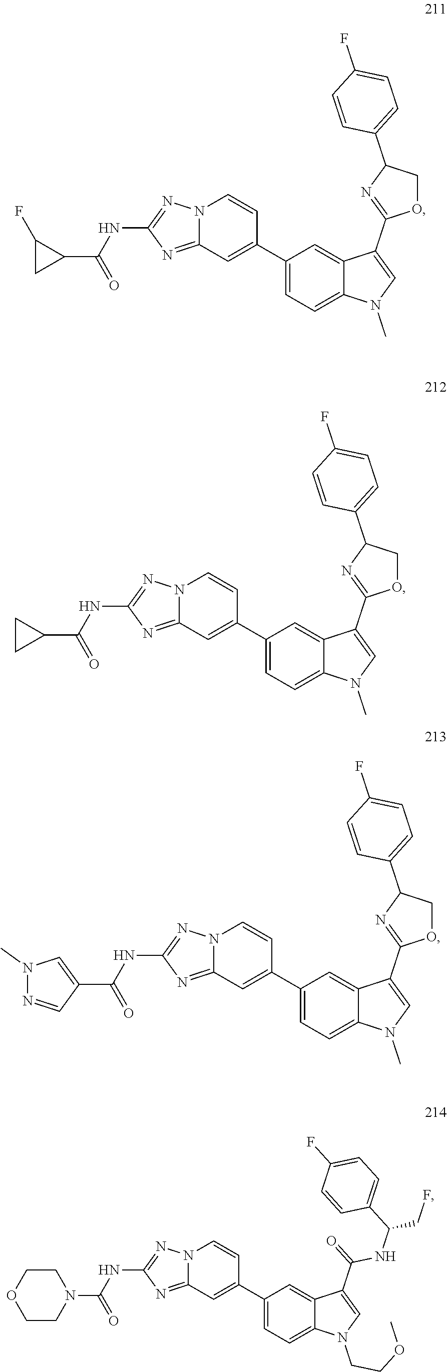 [SMILES: Cn1cc(C2=NC(c3ccc(F)cc3)CO2)c2cc(-c3ccn4nc(NC(=O)C5CC5F)nc4c3)ccc21 ; Cn1cc(C2=NC(c3ccc(F)cc3)CO2)c2cc(-c3ccn4nc(NC(=O)C5CC5)nc4c3)ccc21 ; Cn1cc(C(=O)Nc2nc3cc(-c4ccc5c(c4)c(C4=NC(c6ccc(F)cc6)CO4)cn5C)ccn3n2)cn1 ; COCCn1cc(C(=O)N[C@@H](CF)c2ccc(F)cc2)c2cc(-c3ccn4nc(NC(=O)N5CCOCC5)nc4c3)ccc21]

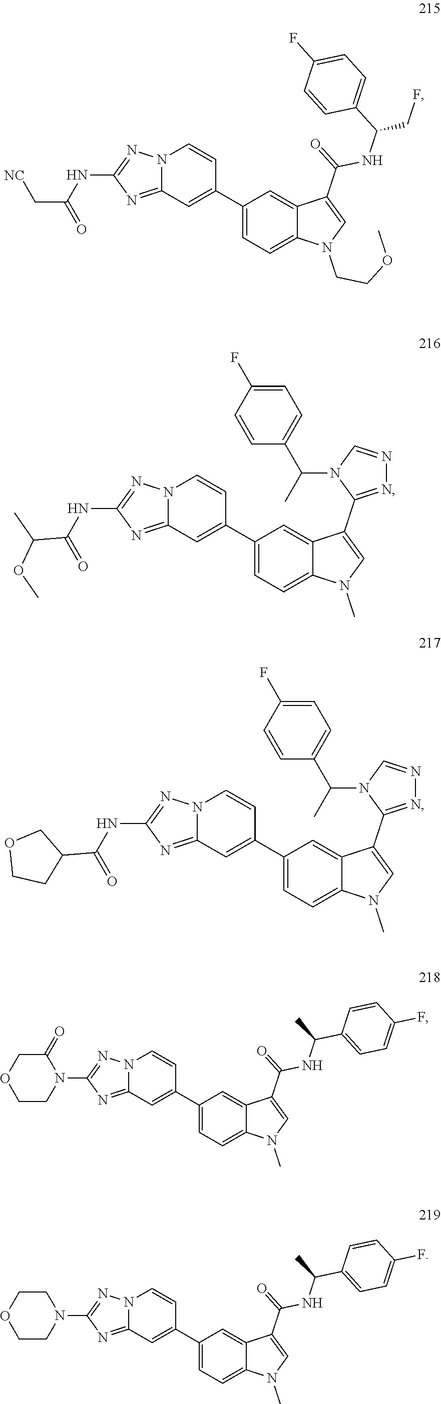 [SMILES: COCCn1cc(C(=O)N[C@@H](CF)c2ccc(F)cc2)c2cc(-c3ccn4nc(NC(=O)CC#N)nc4c3)ccc21 ; COC(C)C(=O)Nc1nc2cc(-c3ccc4c(c3)c(-c3nncn3C(C)c3ccc(F)cc3)cn4C)ccn2n1 ; CC(c1ccc(F)cc1)n1cnnc1-c1cn(C)c2ccc(-c3ccn4nc(NC(=O)C5CCOC5)nc4c3)cc12 ; C[C@H](NC(=O)c1cn(C)c2ccc(-c3ccn4nc(N5CCOCC5=O)nc4c3)cc12)c1ccc(F)cc1 ; C[C@H](NC(=O)c1cn(C)c2ccc(-c3ccn4nc(N5CCOCC5)nc4c3)cc12)c1ccc(F)cc1]

8. A pharmaceutical composition, comprising the compound according to any one of claims 1-7, or a pharmaceutically acceptable salt, solvate or stereoisomer thereof.

9. The aforementioned compounds, or pharmaceutically acceptable salts, solvates or stereoisomers thereof according to any one of claims 1-7 or pharmaceutical composition according to claim 8, in the manufacture of a drug for preventing and/or treating inflammation, autoimmune diseases, neurodegenerative diseases and tumor-related diseases.

10. Use of the compound according to any one of claims 1-7, or a pharmaceutically acceptable salt, solvate or stereoisomer thereof, or the pharmaceutical composition according to claim 8 in the preparation of RIPK1 and/or MLK inhibitors; Preferably, the said MLK inhibitor is an MLK2/3 inhibitor.
